SEQUENCE LISTING (334 sequences)

SEQ ID NO: 1 - DNA, 1893 bp, synthetic construct
  source 1..1893: mol_type = other DNA
atgtcttcatcacatcgtatattgtgtgtggcgttttgcgtgtgttccctagtagcagtgtccagcatatatagtccgccatttaatcacccgattgtatattccaacaatgcaatctgtttccagctttccacttggagaataccccagttgtaccttgccgtgctaataagaaggttcgacctaagtctttctaccaggggagtcatgctggctgtgactgtgggcgttgggtctagccagcaccactcccaaagatccgggttgtctttgactttcgcaatgaccccatctatgattacactactggtgaagacttgcgtaatctcacaacgtactgccgtgggggagccacatcgtccaggctgggtctgggaggtcggaaggacagaggatgagacgtgccatgcgcacagtcctaggggcgctccgtgccatcagggtaggataccgctgtactccgctgccgtcgagtcagtggaccaaattcaagtagctgttagatttgcccaaagacaccgtctaaggcttgtcgtaagaaacacagggcacgacaccgccggtcgtagcagcggttcagacagttttcagatccattgccataggatgaagcaaatcgaataccacgacaatttccgtgctttgggatctgatatcgatagggggcccgcagtaagcgtgggggctggagtaacgttaggtgaaatgtatgctcgtggggcccgtgatggttgggttgttgttggcggagagtgcccgactgttggtgctgccggtggattcctacaaggaggtggtgtaagctctttccactcattcatcgacgggctggccgtcgataacgtattagagttcgaagtggttacggcgaaaggggacgtcgtggtagcaaacgaccaccagaatccagacatattttgggctttgagaggaggcggtggcggcacttttggcatagttactagagcgactatgagagtccatctgaatagtccagtatgcgtttccgaggtcgccgtaagcgggttgcgtaataacagtttactttggaccaagggtatcacaggattattctcaattctgcgttcttttaaccaacagggaataccaggccagtttatcttgaggcctttaagcaaggaccaagtaaatgccagcttgacgttatactcacttaatacagacgatacccgtagatctgccgaaaacatgttgagcataagaaacattttggaatctaccacccttccattcacacttgccagcagatgcctgccgaagatctctgacgcattaagaaagggtcctgatatgttgcccgtcaactatggtatcattaccggcagcgttctggtgtccgaagacctatttaacagtgaagaagggccacttcatcttgcgaaacaattagagcatttcccgatgggaccaatggatcttttattcacgtctaatcttgggggcaacgtatctgctaatacgggtaaaaagcatcgtgacacatcaatgcaccctggctggaggcaggccgcccatctgattaactttgtgagaagtgtaagcactcccacggcacatgaaaaggcccgtagtcttgaagaattacatagcgtccagatgaggcaactgtatgatatagaaccagatttcagggtaagttatcgtaatctaggagatccacttgaatctgacgcggcgcaagtatactggggccccaactataaacgtttgctagagatcaagcgtaagtgggacccagaggacttatttttctcccaattaggcgtcggttctgaaggttggaccgaagatcaaatgtgtaaacgtcaacaacgtctacaacaaatgcttcaatatctgatgtcctcaatagctcaaagagtttaccgttaa

SEQ ID NO: 2 - DNA, 1872 bp, synthetic construct
  source 1..1872: mol_type = other DNA
atgtcttcaggccagtccagaggaattttaggtggcgtgaggcagctgattttggtaattttggtcggggcgtatctatccaggctttccgcggtcgatgacgataggcatgactgccgttgcagacccggcgaaccctgctggccgacagtgagccattggtctactttgaatgaatccattggcggggcccttgcccatgttaaacccatcgcccatgtgtgtcaccagtcaggccaggacagttccgcatgcgagcaagtgcttcaagaaagtttagattccaaatggcgtgctagtcataccggggcactacaagactgggtctgggaaggtggagccgagtccaatcaaacttgctattatttaaggtctggtcctgctggtagctgtcaccaagggagaataccgttatactccgcagctgtcaaaagcgccagcgacgtgcaaaaagtggtggattttactagacaacataatcttaggttggtcattcgtaacacgggccatgacgggagtggaagatctagcggtccggactcagtggaaattcacacacaccaccttaactctgtacaatatcacccaaattttagaccagctggtagctccgaaaggcaatccgctccaggtcaacctgccgttacggttggggccggcattcttttgggcgatttgtacgctcgtggtgcttccgagggctggattgtggtagggggtgaatgtcccacagtgggtgctgcaggagggttcttacaaggcgggggagtgtcatcctggcttagctacgctcatggtttggccgttgacaatgtcctggagtatgaggttgtgaccgcgaagggagagatcgtcatcgccaatgcgcatcaaaacccagacctattttgggccttgcgtgggggcggtggagggacctttggggtcgtgactcaagccaccttgcaagttcacccggatctaccagtgtctgtggccgatgcagttgttaccggaagtcgtgcggatgctacattttggtcccacggggtggcggctctgctaagggcgttgcaattccttaataaccacggtaccgctggacaattcatactacgtaataccgacgacgataccgtgcaagcgtccttaactatgtacttcagtaacttgacggttcccgctgtagctgacgagcgtatggaccctttgaggagggctcttgaacacaacggccatccctaccagctgaccagtagattccttccccaaatctcctcaacttttagacacacggccgatagataccctgaagactacgggattttgatgggctccgtattggtgtcacttgatttgttcaactctgcgactggaccagctgctttggcacaacatttcgcaaggttaccaatgacgccagatgacctacttttcactagcaacttaggtggaagagtaagctcactgaacagggacccggccagtacggcgatgcaccctggttggagggatgcagcccaattactgaactttgtaagaggtgttggggctccatctctagctgctaaggccacggccttgcatgagctgcagacggtgcagatggcgcgtctgtacgagatagagcctgcatttcagataagttatcgtaacctgggcgatcctagcgaacgtagaagtagggaggtgtactggggaagtaactatgctagattggttgaagttaagcgtagatgggacccggaaggtcttttcttttcaaagttagggatagacggcgatgcatgggacgccgaaggtatgtgcaggcaaacgaggcagggagcttggaatgttgccgttaagtgggtacaaagtttcttggggtcactaagtgcttccgtggtttaa

SEQ ID NO: 3 - DNA, 1794 bp, synthetic construct
  source 1..1794: mol_type = other DNA
atgtcttcatactacctgctaggccccgtggcctgtttcgccctaattgcctccgtcctttgggctccatcagggaggtgccgttgccgtccgtgggagccctgctggcccagtgccgctgattggcatgtcctgaatgactccttgcaaggctcccttgttaggatacgtcctgttgcatcagtatgccacggttccgaatatgatgtagccgcgtgcgcaaacctttctagtctagtagtagatagtggctggagagccagtaatccaaacactcttcaagactgggtctgggaaatagggaacggagtcgaggagttctgtccctacacaatttctcctatggaacgtagacaaagcatctgcccacaagggaggttgccgttttattcagccgtcgtgcgttctacgtcagatatacaggcatctgttaggttcgcgtcaaggcacaacttacgtttagtaatcaagaataccggtcacgacagcgccggccgtagttctgcaccccactcctttcagatacacacgtctctattgcagaacataagtctacacaagaattttatcgcacgtgggtctacgacgggaaggggacccgcggtgacccttggcgctggagttatgcaatggcaggcctatgtccacggagctaaaaacggttacacgatcttaggaggggagtgtcccactgttggcgcgattggcggtttcttgcaaggcggcggtgttagtagcattcatagttttaccagaggacttgccgtcgatcaggtcttagaatatcaagtcgtttctgcgaagggtgacctaataacggccaatgaagataacaatcaagatttattttgggcgctgaagggtggagggggcggaaccttcggtgtcgtaaccgaagccactgtaagggtgttctctgatgaccccgttacggtcaccagcactaagattgaagcagctgcagcgaatgtcttgttttggaaagagggcgtacacgagctactaaggctgttacaacgtttcaacaatttgcacgtcgcgggccagttggtcatatcagcgcctactaaagacagcttacaagcaggtctggaattacactttgcaaacttgaccgacgagacccaagcaattcagctactaagatcagaagcaagagccctggaaactcatgggatatctgcatctacgtccgtaagggtgcagagaaaagctagtagcgagttgagaatgaagcctgatctgtatccaccgcactatggcatattggaagcatctgttcttatttccgcagcgaccttccacgcgaatgatggacccgctctgatcgcttcaaaactatctggtttaactttgaagcctaacgatatactgttcacttccaatctaggcggcagagtctccgagaatacggcgatagagattgcgctgcacccggcgtggagagaggcggcacaactggtcactctagttagagtcgttgaaccgtccattgagggtaaattgtccgcgttaaatgacttgactgctagggacgtgccgatattatacagtattgatcctgcggcaaaaatcagttacagaaacttaggagaccctcaggagaaagagttccaagcacgttactggggagcggataattatgcccgtttagcggctacaaaggcagcgtgggacccctcacatttattcatgacgtcattgggtgttggctctgaagtgtgggatgccgaaggtatatgtaggaaacgtcgtggattcagagcgaaagcctcctcccttatcggcatgtaa

SEQ ID NO: 4 - DNA, 1884 bp, synthetic construct
  source 1..1884: mol_type = other DNA
atgtcttcagcccttcaccttgcgctgtttctaacagcagcggctttacttgtggcttttgcccagtctcaaactctttttggttatgaaaagacgcaattgactcgtaaatacgtcgcgagtttgccccaggaggatgcaaggctgttcgcattcgaagaccaattcgaggacaggttcgaagccaccgccgtatccgcaactgagtgtaaggttaatcctacggacaagaaatggcccgcagagagagcctggaagcgactagaaagtcgtctttcttcccccaacaatctgatcgctgctgttccgcaggcctccgtctgctacgcgccaacaagggacgacgcacagtgccagcagcttacgaggaattggactaatagctacacgcatatagatgatcccacagaagtcttgtcacctatctaccagggcctaacttgtcaaccacctctaatctttgattcaggcaactgtacaataggaggttacccggcctacgtcgtcaaagcaactgcggtaagtgacatccagaccgcgattaacttcgccaggaacgacaatcttagacttattgttaagaacacggggcacgacttcgcagggcacagtgtgggttacggtgcactatcaatatggacacatgtcctaaaggatatgatgttcttcgataactatgttgatgagagtggttataaaggccctgctataaaagcgggagctggcgttcaagcgtttgaactttataaatttgcaaacgagcatggggtcgtcgctgtagctggtgaaggccaaacagtaggcgttatgggtggattccttttgggaggaggtcattccccattatcatcaatttacggaatggcctccgaccatgtgttgggattcgaagtcgtgacccccataggcgagtttttgaccgcgaacagtacctctaacacggaccttttctgggcactaaagggtgggggcggcgggacattcggagtcgttacttcagtgacgttcaaggtatacccagatatgccggtgacgaccgtttcttggaccctggatagctccaagataggcaaggataagttctgggcagcaaccaaagcgtttttcgatagagcaattgataatgttgatgcggagacttactcttataactccattattccgattggcgaaaacttcttatcaattatgcaaccgtttttcgctcctaataaaacagcgacccaactaaacaccattctggggccctatttcagcaagttaactgctttaaatataccgttctctccgaagacgacagaatacaagacgttctatccagcttggcaagcagagtttccccttgaacccatgagtggcacgaactccgcgacggcatctaggctgtttcctaggagcaatttcgtctcagacataggtaagacaaccaccttcgccgtactgagacagacacttgaatccggcaaaccactaatcgcatttaatatggctccacgtcttttaccctccaatccggacaatgccataaatccggcatggaggacttcagtctatcacattataaccggcgctaccatcgacccgaggtcatcccccgcgactcaattggccgcaagggcggcgcttactaacggtacaatgcgtgcttggagggatgtcacaccaggctctggtgcatacctgaatgaagctgacagattagaacctgattggcagaggagtttttggggcgccaattatgaaagactggtcggcattaaaagggagatggataggagagacgttttctgggtggatcgtggggtaggaagcgaaggctggcgtgttgaatctgtagatggtgtacccaatgaaaatggcccactgtgtagggttggtgggtaa

SEQ ID NO: 5 - DNA, 1809 bp, synthetic construct
  source 1..1809: mol_type = other DNA
atgtcttcatataatctgttggggcctgtggcctgcttaacattagcaggacttgttctgtggcttcactctggtcactgcaggtgtagaccctgggaaccatgttggccttccgcagctcactggaaggcattcaacgaatccatccaaggaaacttagtgagaatcagacccgttgcgtctgtctgccatacgttagaatatgatgcggttgcgtgtgctaatcttagctccttagtagtcgatagtggctggagagcttcaaatccctctaccctacaagattgggtctgggagtccggtaacacagctgaagagacgtgtcactcaatgacgtcccctggcgagcagagaagaagtgtttgtcatcagggcaggttacccttgtattccgctgtggtacaatcagctagtaatattcaatctagtgtgcgttttgctgctgaccataacttgagactggtcgttaagaacacagggcatgattgtgccggcaggtctagttctcctcactctttccaaatacataccaatctgttaaagaatatcagtttccatgagaactttgttgccggaggttcaacgactgggtatggtccagctgtcacgctaggggcaggtgtcatgcattgggaagtgtatgcccacggagcagaaaaaggctatacaatcctaggtggggagtgtcccactgtcggagcggtcggtggatttcttcaaggtggcggagtgagttccatacatagtttcaccaggggtcttgcagtggacaacgtgcttgaataccaagtggtcactagcaatgcggatcttgttaccgcgaatgaacacgagaatcaagacctgtactgggcgctaaaaggcggaggaggaggaactttcggcatagtaacccaggccaccgtaagagtctatcctgacgatcccgtcactgttaccacgactgtaatcaaggcggccttcacaaacacattgttttggaccgaaggcgtaagagaacttctaagactactacgtgacttcaatctgctgcatattcccggacagttggtggtaagggggccggaaaagcactcaattcaagctacgctggagctgcattttgcaaatatgacagacgaagcgtatgcaatgaaattgctatccagcgaactgcgtcccttgagtattcatcacattagcactagcacatctgcgagggtgcagaagcgtgcgtccagtgaactgaggatgaagccggatatataccctccccattatggtattctacaagggtcagtgttgatatcaactgctgtttttcaaagtgccgaaggacctactttattagctagaaaactgtcagaattgacaatgagattctcagacattcttttcacctccaacctgggaggacgtgtatcaaagaacattgctatagacatcccgcttcatccagcatggcgtgaggctgctcagttggttactctagtacgtgcagtagagccctccacgaagggaaagctatccacactggaaaatctaacttcccgtgacacccctattctgtacagtatagacccctccgctaaaatttcctaccgtaacttaggagatccccaggagcaggaatttcaagcgaagtactggggcgccgacaattacgcaagacttgccgcaatcaaggctaaatgggaccctaatgaactattcattacgagatctggggtaggatcagaaaactgggacgtcgagggcatatgtagaaagagccaggacttcatcgcgcaggcttttgctctgctaaaagttggactaagtagtcattaa

SEQ ID NO: 6 - DNA, 1869 bp, synthetic construct
  source 1..1869: mol_type = other DNA
atgtcttcataccagctactggccccagcagcggctcttgctctgggtgtagctgtgtttaggtcacagccgaatcaacatcagtgcaggtgcagaccttccgaaccttgctggcccagtgcagaccaatgggacgtttttaataagagtataggtggcaacttggctaggatcaggccagtcgcaagtgtgtgccacggcccagaatatgatgccgccgcttgcacggctctatcttctttggcgagggatagcgggtggagggcatctaacccggccacactacaggactgggtctgggagggaaatggcctagcggaagaaatttgtcacgtggtgacatctcccgaggaaagccagcaatctgcctgccaccaagggagagtaccactgtactctgctaccgtacaatctacgactcaaattcaatcatctgtgcgttttgctaagaagcacaaccttaggctagtggtcaagaacagcggacacgactgtgctgggcgtagttcctctccggacagctttcaaatccatacgaaccttctaaagggcattacatatcatgataatttcgttgctcagggtagcgcagcaaacgggtctgggccggccgtgactatgggcgctggaatcatgcactgggaagtctattctgacggggcgaaaaatggctacacggtagtgggtggcgaatgtcctacggtgggggccgtcggcgggttcctacaaggtggaggagtatcttccttccactctttcactaaaggcttagctgtggacaacgttctggagtatcaggttgtaactgccagcggggaactgattgcagctaatgaatatcaaaatagagatttgttttgggcacttaaaggaggaggagggggaacattcggagttgtagctcaggcgacggtgcgtgtgtacccggacgatccaattacagtagctgcgatcagcatttctacgagcttgactaatagtctattctggaccgagggtgtctccgagctattaaggttgttacgtatatttaaccaacaagggtttccaggccagatagtggtaatggggccgaaccaaggttcagtgcaggcgaccctggagtttcatttcgccaatacgacggatgaaagctatgtgaacggcttacttaatagtcaaatgaagtcacttggcgaccacaaaatctctaccaatttatccatccgtgttcaaaagaaggcctctagtgaggttcgtctgggccctgatacttacccaccacagtacgggatagttcagggaagcgtgctgatcagtgcggcattgttcaattcagcagatggccctccactaatagcgaggaatttcagcaacttgaacttggggccaaatgacattttgtttacgtctaacctgggagggagggtaatggacaactctgacatagatatccctctgcatccttcttggagatctgcggcgcagctagtgaccctagttagggctgtggaaccatccaataagggtcgtctggctgcgctggagacacttcatagtcacgacatgccaatattgtactctcttgaaccaaagaacaaagtaagctaccgtaatcttggggacccctcagaaagagagtttcagagcagatactggggtagtaattacaaaagattggcatcaatcaaggaaaggtgggacccagatgaacttttcataacgagattgggagtgggatctgaggaatgggacactgaggggatgtgtagaaagtccattggtctagtagcgagagtgtctgaactgctgaggctatcaggtagatggattctaggtgggttcagcgcgttaatccagcaggtcgatattatcgactcatccaggtaa

SEQ ID NO: 7 - DNA, 1821 bp, synthetic construct
  source 1..1821: mol_type = other DNA
atgtcttcatcatgcaggtgccagccttgggaaccgtgctggccgtccgatgagcagtgggaattattaaacagaagtatggagtttactcttgtcagactacagccaatcgcgtccgtgtgctacaatcattctttggatcacaccttgtgcgaggacgtaatgagaatgagtagagatagtggttggagagccagccagcctggagccctccaagattgggtgtgggagagcggccaaagtcctggagatgcttgcccgctaggcagtcagatgaacggacatacgcagtgtcaccaaggaaggattcccctatactccgcgatggtcaattcaactaagcatgtccaggaggcggtaatgtttgcgaagagacatgacttgcgtctgataataagaaatacgggtcatgacctagccggcagatcttcatcccctaatgcgctgcagattcatacccatagattgcaggatataaaattccatgataacgtacagttacacggttttgaaaaatcttttggtccggctgtatctgtaggtgcgggtgtgatgatgggtgacttatacgcccgttctgcacagaatggttatatagtggttggcggggactgtcctactgtcggtgttgttgggggattcttgcaaggtggtgggatttccgactttttaagtcttcaccacggactggcggtagataatgtcctagagtttgaggtcgtcaccgcctctgtatccttaccagaaccggacgcgatacaatactgcggtttcctgacgttaaggcaaggtgatattgtgttggcaaacgctatcaggaacccagacctgttttgggctttacgtggaggcggtgggggtacgttcggcattgtcacacgtgcgacgatgagggtatttcccgatgtaccggccattgcagcggaactgggggtgcagacgtcccagtcacatggtgaatattctaggagcttagccgcattcttcacagtgttacagtcactaaatagagaaaacgtgggcggtcaacttatcataaccgtaatttcagagcattcaatagaagcaaagctgaagatgttctttctaaatcagaccaataccgctgatgtagaccagagaatgcagcccttcgttgaggatatgcgtagaatcgagactcatgttacttacgaaagcacgtctttaccgaaactgtcaatgaattaccgtcaggtgcccgacatccatacggacaacgactacggtgtattaggatctacagttgcaattagcaacgacctttttaatgcttcaaaggggcccgcttatgtggccgacggtttagcccagttacccactagacctggggacctattgttcacgtctaacttagggggtcaggtgatgagaaatggcgacttgatggaaactagcatgcatccggcttggcgtaacgctacacagttaataaacttcgttagaccagtagagcccacgatagaaggtaaggcaggcgccctgcaaaatctgaccaacatccacatgcccctactgtatgcgatcgatcccagatttcgtctaagctacagaaacgtcggagatccaaacgaaaaagattttcaacaagtatactggggtcagtcatatgcaagacttttacaactaaagaggaggtgggacagagagggtctgctgatttctaagctaggagttggtagtgaagaatgggactctgagggaatgtgtcgtaccggtagaagaagtcttgaaaatttggcaatagatacactgagtagtttagcgcatcttatccatgttacgtatcgttgcatttggtaa

SEQ ID NO: 8 - DNA, 1809 bp, synthetic construct
  source 1..1809: mol_type = other DNA
atgtcttcacatagacttttggggcctgtggcatgtataacgttagccggggccgttttctgggcacaatccggtcactgtcgttgtaggccctgggaaccttgttggccgtctactgcgcaatgggaagcatttaatgaatcaatccaaggcaatctggttaggataagaccaattgcatccgtctgccacggtcttgagtatgacgcaatcgcgtgcgcaaacttatctaatttgatcgttgacagcggttggagagcgtcaaacccgagtaccctgcaggattgggtttgggagtccggcaatgtagccgaagaggcgtgccacattatgacttctccaggggagcagaggggctctgcttgtcatcagggcagattgccgttgtactctgccgtcgtccaatcaacttcagacatacaatctagtgtaaggttcgccagtgaccataatcttcgtcttgtagtcaagaacacgggacacgactgcgcgggacgtagtagtagccccaactcattccaaatccacaccaacctactgaaatccattagttttcatgagaacttcgttgcaagaggttccacgacatgcagtggacccgctgtgaccttgggcgctggtgtaatgcactgggaagtctatgcgcatggcgtggagaacggttacacgatcgtcggcggggaatgtcctacagtgggagcggtgggcggctttttgcaaggtggtggggtttcatctatccatagtttcacgaagggtttagcagttgataacgtattggaatatcaagtagtcacgccgaatgctgatctagtcacggcgaacgaacatgagaatcaagacctttattgggcattaaagggtggagggggagggaccttcgggattgtaacacaggccactgtcagagtctatcccgacgatcctgtcgtggtaacagcaacaatgataaaaactgcctttgcgaatactcttttctggacggagggggtccgtgagctgttgaggctattgcagtactttaaccagttgcacatccctggtcagttggtcgtcactggacccgaaaaagacagtatacaggcgacacttgaattgcatttcgcaaacatgaccgacgaagcccatgctatgaaactgttaagttcagagctacgtcccttgtcaatccatcatatatcaacgtctacaagcgtgcgtgtgcagaagcgtgctagtagtgaactgagaatgaaacccgacatttatccccctcaatatggaatcttacagggcagtgtcttaatttcaactgcagtttttcagagtgcggaggggcccacactaattgcaaggaagttctccgaacttaccgtgcgtagcaacgacatattgtttacatccaatctgggcgggcgtgtttcaaagaatatagcgatcgacgtgccgcttcacccagcgtggcgtgaagcagcccaacttgttacgttcgttcgtgcagtaggtccgagtatagaagggaagttaagtgcgttagagaatttgacgagtcaagacattcctattctgtattcagtggacccaaacgccaagattagttacagaaatctgggtgatcctcaagaacaggaatttcaagctagatactgggggcttgacaactatgcacgactagcagccatcaaggccaagtgggaccccaatgggcttttcatcactagaagcggtgtagggtcagaagactgggacgtagagggaatatgtaggaagtctaggggcctaatagcccaagcgttagaactactgaaacttggaccatacatatactaa

SEQ ID NO: 9 - DNA, 1638 bp, synthetic construct
  source 1..1638: mol_type = other DNA
atgtcttcagaggacggtgggtgtaaatgctttccggggaacgcatgttggcctggacactctgattgggatagattgaacagtacagtgggaggacgtcttatagccactaccccactaggcagcccatgccatgaccctcattataatgaaaccgagtgtgcaacccttcagaaggagtggttatactccgcagtgcacatggaaagttcttcttctgtgatggctccattgtttgctaacaagtcatgcgaccccttccagcctcgtagttcaccatgtacattaggtaactatgtcagatatgctgtagatgcgaggagtccacaagacgtagccgcaacgcttacttttgcctcccgtcatcgtattcgtctggtggtaaggaacactggacacgactataatggaaggagtacgggagctggcgcgttagctgtatggacacaccatatgaaggactcagcggttatcgactgggcggacagacactatattggtaaggctctaagagtcggagcgggagtacaggggttcgaggctctggctgaggctagaaagcacggaagagttgtagtgacaggggagtgtccaaccgttggtctggccgggggatacacgcaaggaggcggacatagcgccttatctaccgttcatggacttgctgcagacaatgtattaagctacgaggtagtcacagccagcggtaaacatgtaagagcttccagggcggagaacagtgatttatactgggcgctttcaggcggtggcgggggcaactacggggttgtagttagcatgacagtgagagcgcatcccgatgctaaagtatccggggcatcttttgccatcacggcgcccgaaaatagcacatcactttatgacgcaatagaccttttccatgaagcattgccagggatggtggattccggagcaatggttatatattattttggggcaggattcttgcagatgcccgcactaacactgttcggcaagagcacagaggagacagagaaggtcctgaagccctttattgatgccgcatctgcgcaagggtttgtagttaaacccaattacacagagtttgaaagctactatgatcactatgcgcactattgggggcctctgcccgcaggcaatatccaagttgggacacagctatttggtggaagactgattccccgtgatgcacttacccactttagtcctacagcgagagagctagtctcaatgggtgtgacatttatcggtgtcggcctggatgtgagtcgtttcggtagagaaaaagcggctgcggtgctgcctcaatggcgtcagacactggtgcacgcgtccctgactatgccttggtcattcgatgccccctttgaagagatggtggcggagcaaaatcgtatgacacgtgaggttcagcctgtaattgaagctgcgacgccaggggcgggtgcgtatatgaacgaggctgattttcaacaacctgattttcaagagacctttttcggcgctaactatgatcgtctactgaggatcaagaagaagtacgatccgaacggtctcttttatgctaccgcgggtgtgggttccgaggagtggacagtcaagaaagacggtcgtttgtgcagagcccatcgttaa

SEQ ID NO: 10 - DNA, 1734 bp, synthetic construct
  source 1..1734: mol_type = other DNA
atgtcttcaaggagtactgcgtatcttacggctcttttgagctttctaggtgccacctacgcggctcctgaaggccctcgttgtagatgcacccctgaccagtcttgttggccaagccccggcctttggcaaacactgaacaagaccttgagcggaaaccttgtcgcggtcaaaccagtcggagcggtttgtcatgacccgacctacaacggcgggctttgcgactcagtcaaaggggccgtacaatccatcaactgggagacatggcctgaaaagaatgagtcatgctatatcgacggaccaaggcaggtaccctgtgggcagggcagaataccattgtacagtgctgtcgtccaatctcctctcgatatacagaagacggtgaggtttgcgagtaaatacaatttaaggctggttataaagaacaccggacacgacttcctagggagaagtactggaccccagagtttgcaaatcctgactcacaatatgaaaagcatcaattttacagacaattttgtaccggaggggaagcccgacggtaggggtataggacaggctgtgacgatcggtgcaggggttcagttaaatgagttgtatgaagcagctggaaagagagggcttacccaagtgattggactgagcaccacggttggtgcagccggtggctacatacaaggcgggggtcactcaccgcttggcccgtggaaaggcatgtcaacggaccatgtactggagtacaaggtcgtgaccgcaggcgcgaaattcgtaaccgctaacgagtaccagaatagtgacctattctgggctttaagaggagggggaggtgggacgttcggcgttgtaacctctgtgactttgcagacattcaaagatcccccaaccatcgttagccaggtgaatattacgatggacggcaaggcgaatgaaagctactgggcctccgttgaaaaatttcaggcgtatctaccaacgctttccgatggaggctgttctgggtactattacatgctacccaatgttacacttgggccgcaaagtgctgcggtaatcatagccgcattttattacgcgaataagactgacaaagcccatgtggacaatctatacagaccgctgttcgcctccctttcctctataccaggaataaacgtggcatcagtaagtgtgccagtgtcctcctcaactgaggcgttcagaagtgccttcgatcaaaagccgccgcgtgacggaggtggcgttaacatcctagggagccgtctattctccaggaaattactggaaacccctggcggggcggctaacctaaccactgcattgagtaagttggacttcaagaatctgcagcctgccattggtcacctagtagctggtgggcaggtggctaaaaatactcacattcagtccgcattgaatcctagttggcgtaaggccctggtccacctggtaattagtagggactggtccatcgatagtacatttgctgaacaagagaagattagtacgaaattaacagcagaggaaataccccttctagctgccgttgaaccggatatgggggcgtatactaacgaggcagatgttaacgaacccagattccagcagactttttgggggacaaactataatacgttgctgagggtgaaaaataggtgggacccgagaggactgttctttgtgagaagtggcgtaggtagtgaggcatgggacaagcagggtttatgtagggtgtaa

SEQ ID NO: 11 - DNA, 1821 bp, synthetic construct
  source 1..1821: mol_type = other DNA
atgtcttcacaatttcttctatggtcaactggcttggttgcgctgttatcatggttgatctatacgcaggaaactcaaagcgcctcttgtcgttgtaggccctgggaatcctgttggccgtctgaagaattatggaatagcttcaacaccagcgtagatggcaagctgcatagattacgtccggcggcccatgtttgttacggccctagtttcaacaggtcagcctgtgataatattcttctattgagtagggattccggttggagagccagcaatccaggggtgttgcaggattgggtgtgggaagcaggtgagacggcgaacgagtcctgtccagtgggctcactgaggactgcgagtgctgtaaatagttgccatcagggcaggatacccttatttacagttggtgtggaatccacaaagcaagtgcaggaagcggtcaggttcgctcgtaagcataagctgagactggtaataaggaatactggacatgacctggctggcaggtcttctgcgcctgactcttttcagatccatacgcataggctgcaagagatccaatttcacgcggatatgcgtttggatggctccaacacgtcacttgggccggccgttacggttggcgcaggggtcatgatgggcgatctttatgctcaggcggcgcgtcatggctacatggtgttaggaggggattgtccaactgtcggtgtcgttggagggttccttcagggcggcggtatctccgacttcttgtcccttaaccagggtttcggagttgataacgtattagagtatgaagttgttactgctgatggtgagttggttgttgccaatgctctacagaaccaggaccttttctgggctttaagaggcgggggtggtgggacttttggagtcgtcactcgtgctacaatgagagtgttcccggatgtccctgtcgtcatttcagaaatattgttagaggcgccgcaggccataagttcctcttggacccaagggctttccattgtcttaaccgccttacagtccctgaaccatgacaacgttggcggtcaattagttatcgctgtgttgcccaacttagcagtccaggcgagcataaagttcttcttccttgacgcaactgaggcagcggttatagacagaaggatgaagccctttctgacgaaattatctagggcgaatgtgaaatacacttacagtagcaagaatctacctcatttctctagcaactacagacaagtgccagacatccattctgacaacgactacggcgtattggggtctactgtagcgatctctcagcagttatttgattcacctcaaggcccggagaaagttgccaaggctttggcaaatctgccagttagtgctggagacttaatattcacgtcaaacttgggtggtagagtgatacgtaacggcgagctggcggagacaagtatgcacccggcttggaggagtgcttcacagttgatcaattacgttcataccgtagaaccaagcatagagggtagggcgaaagcaagggaacgtttaactaatacgcagatgccgatgttgtatgctttagatccgaacataaaactttcatataggaacgttggcgatccgaacgaaaaagatttccagcagatctactggggtccaaactacgggagactgagcaacattaagaaaaagtgggataccgatgatctattcttctccaaacttggggtcggttctgagaggtgggattctgaggaatctgatgaggtctatttctttagttatttctaccttgaggggatcaacaccttaaaagttgccaggcgttaa

SEQ ID NO: 12 - DNA, 1536 bp, synthetic construct
  source 1..1536: mol_type = other DNA
atgtcatccgcgccggattccaagcccacgtacaccactgcgaatggttgtccgtttcataaacctgaaggtccgccggctgatggcagaacgctggccttgtcagatcatcatctggttgatacattagcccatttcaatagagagaaaattcccgagcgtgtagtccatgccaagggggcgggtgcgtatggtgagtttgaggtaaccgcggatatctctgatatttgtgacatagatatgttagtaggcgtgggtaaaaagacaccgtgcgtaactcgtttttcaacaacgggccttgagaggggatctaatgaaggcatgcgtgacttaaaggggatggcctgcaaattttatacgaccgaaggcaactgggattgggttatgctgaactttccgtttttcttcatcagggacccggtaaagtttccgagtttgatgcatgcacagcgtcgtgatccgcaaactaaccttctaaacccaaatatgtactgggattgggtgactaacaaccatgaatccttacacatggtactgctacagtttagcgattttggcacgatgttcaattggagaagcatgagcggttacatggcccacgcttacaaatgggtgaagccggacggatcttttaaatacgttcacatcttcctgagttcagatcgtggtcccaacttcactgatggccaacaggcgaaaggtacaaatgatttggaccctgaccacgccacaagggacttgtacgaggctatagagaggggagaatatcccacgtggactgcctccgttcaagtagttgatcccaaggacgcccctaatttgggctataatatattggacgtaacaaagcactggaatttaggcacgtatcccaaagacgtaacgttaatccctccgaaagtatttggtaagctaacgttaaaacgtaatccggcgaactattttgcagagatagaacaactggcctactccccatcaaacatggtccctggagtggcgccgtcagaagatccgattctgcaagcccgtatatttgcctaccccgatgcccagagatataggttaggggcaaaccaccaacaaataccagtaaaccgttctgcccatacttttaaccccatagcaagagacggacaaggcaccttcgacgccaactatggggcacaccctgggtttctgactcaacagcaacctgtgaggtttgcggaaccaagggagcccgatccaaaatataatgagtggttaaaggagatacagtccaaaagttggctgcaaacgactgagcatgactacaaatttgccagagacttctatgaagttctacccgattttcgtggccaggagttccaggacacgatggtccaaaacatggtagagtccgtagctcaaaccagggctgagattcaaaagcaggtctatgagacgtggaaacttgtgagtcctgctctggcagcgcgtatccaaaaaggagtggagacccttctacagaaaagtgaagcgccggcagaggagagcttcataccggcacgtttataa

SEQ ID NO: 13 - DNA, 1428 bp, synthetic construct
  source 1..1428: mol_type = other DNA
atgtcatccgcttctgaggttagcgtggcgtcttctgggtctgaacattcaggggcacaaaaatgtcctttccaagatcctggtttaagttctatggatcaggactcacgtctgagggacattctaagtaggttcaacagggaaaagatccccgaaagggcggtccacgctcgtggcgccggcgcttacggtgagttcgaagtgacacacgatgtctcagacatctgcgacatcgacatgcttttaggagtcggtaagaaaacaccctgcgtcgtgagattctctactacgaccctggaacgtgggagtgcggaatctgttcgtgacgtaaaaggtatggctatcaaacactttacgcaggatggcaattgggactgggtttgtctaaacatcccaatgttcttcatcagggacccgtctaagtttcctgatatggttcatgctcaacgtcctgacccgacgacgaatgtagcaaatccaagcaggtggtgggagtttgtttgtaacaaccacgagactcttcatatggtcatgttccaattttctgatttcggaactatgtttgactatagatctatgagcggttacgcagcacatgcgtataagtgggttatgcctgacggttcctggaagtacgtgcactggttcttggcatccgaccaggggcctaatttcgagacaggtcaccaagccaagcagatcggagccgacgatgccgagtccgcaaccagagacctttatcagtcactagagcgtggggagtatccatcatggaccgtaaaagtccaggttgttgacccggaagatgcgccgaagctgccctttaatattcttgacgtgactaagcactggaaccttggtaactacccacctgatatcgacgtgattccgggccgtaccctgggaaagctaaccctgaagaaagggccccaggactacttcgaagagatagaacaactagcgttttcaccatccaggttggtgcacggggtagaggcctctgaggacccgatgctacaggcccgtttgttcgcttaccccgatgcccaaaagcatagactaggcccgaacaacttggacctgcctgccaatagaacaaagaagctggcagacggtagcaggccggaaaaggcagagatggcgccgcaaaaagtgccttcccaagaacacgcggattgggtaagtcaagttaagagcagctcatggtctgaacccaacgaaaccgactataagttcccgagagaattttggaaagctctgccgagattgcgtggcgaggcctttcaaaattctttggtcgtgaatatggcgaaatccgttagccaggtcccagcggatatgcgtcaaaaggtctacagcacgttggctctgattgccgatgatctggccgatagagtgagaactatgacagaagagattgtggagtaa

SEQ ID NO: 14 - DNA, 1434 bp, synthetic construct
  source 1..1434: mol_type = other DNA
atggcagccccctcaaacatttctgtacaaaccaggggatacggagactctggcagtcagagcggtcaggttaaccaagcatattccgctattgactataggttattagacagcctatcccgtttcaaccgtgaaaaagttccggaaagagcagttcacgccagaggcgcaggggcgtatggcgagttcgaagttactgatgacatttctgacatttgtgatatcgacatgctgctgggggtggggaagaaaaccccatgcgtagttaggttcagcactacaacgctggaaagaggctctgcggaagccgttagggatgttaagggcatggcaataaagttctttactcaggacgggaactgggattgggtatgtctgaatatacctatgttcttcattcgtgacccgagcaaattccctgatttgattcacgctcaacgtcctgatccgaggaccaacctaaccaatcctagcaagtggtgggattttgtatgcaaaaatcatgagacattgcatatggtcatgtttcaattttccgatttcgggacaatgttcgattatcgttctatgtccggttatattaatcacgcctataaatgggtgatgcctgacggcacttggaaatacgtccattggtttcttgcctcagatcaaggtcccaatttcgagaaaggggaacaggcgaaagtcatcgcggcggacgatactgaatccgccacaagggatttgcaccagtcactggagcgaggggaataccctacctggacagttaatgttcaggtcgtagatccagaggatgcacctcagcttgctttcaatatcttagacgtaactaagcactggaatctaggtaattatccccctgatatcccagtcataccaagcaggcgtctgggtaaattgaccttgaagcagggccctaagtcatacttcgaggaaatagaacaattggctttttcacctagccatcttgtacacggagttgagcccagcgaggaccctatgcttcaggcgagattattcgcgtatcctgatgctcaacgtcacaggctagggccccagaacttggatattccagcaaatagaagccgtcacacggtggacggtgccaaaacaaagaagacagtaccgccggagaagaaatcccctgaacatgctgaatgggtctcccaagtaacgacgagcagttggtcacaaccttatgaagcggactataaattcccgagggaattttggaaggccctgccgcgtctaaggggccaggagtttcaggataacctagtggttaacatgagtaaatcagtcagccagataccagtggatatgaggcaaaatgtatacaatactctgggtctagtagccgaagatttggctgatagggtacgtatcatgacggaggaaatgatggactctgaaaaaggcgtccccttgtaa

SEQ ID NO: 15 - DNA, 1569 bp, synthetic construct
  source 1..1569: mol_type = other DNA
atggcagccttaattgagcgtggactactttccataatggccagtaagtgctctggaacgtggtccacgtacaaaacttacacgacagccaatgggtgccctttcatgaagccagaggggcctccggcagatggccgtactttggcactgaatgatcaccacctggttgagagcttagctcactttaatcgtgagaagatccctgaaagggctgtacatgcaaaaggcgctgccgcttatggggagtttgaagtaacggcggatatatccgatatatgcaacattgatatgctacttggggtggggaagaagacgccctgtgtcactagattttcaacgacgggtttagagcgtggaagtgcggaggggatgagagaccttaaaggaatggctacaaagttttatacgaaggaaggcaactgggactgggtttgtctgaacttccccttcttcttcattagagatcctttgaaattcccaagcctaatgcatgctcagaggagggacccaaggactaatctattgaatcctaacatgtattgggactgggtaacatccaatcacgaaagcctgcatatggtcttgttgcagttctctgacttcggtacaatgtttaattggcgtagcctgagcggctacatgggtcacgcctataagtgggtcatgcctaatggatcatttaaatatgttcacatatttcttagtagcgacaggggaccgaattttagccaaggtgaacaggccaaagataattctgacctagatccggaccacgctacgagagatctatacgaagcaattgaaaggggagattatccaacttggacggcgaatgtccaagtagtagacccagccgaagccccggacctgggattcaatatacttgatgtgactaaacactggaacttagggacgtatccaaaggatttaccaaagataccatcaaggccttttggtaagctgactctaaaccgtatcccagacaatttctttgcggaggtggagcaattagcgtttagtcctagtaacatggtgccgggtgtgttgccaagtgaggaccccatcttgcaagcccgtatgtttgcctaccccgatgcacagaggtatagattaggaccaaaccatcataaaattccggttaatcaatgtcctatgaccttcaatccaaccttgagggacggtaccgggacttttgatgcaaattacggcagccttccaggatatgtcagtgagagtcagggggtaaactttgctcgtccccaagaacatgacccaaagttcaatgcgtggttgtctcaattaagcagtaggccctggatgcaaacaaacgaaaacgattacaaatttcccagggacttttataacgccttacccgagtttaggtcacaagagttccaggacaagatggtggaaaacataatagccagcgtcgctcaaacgcgtagggagattcgtgaaaaggtgtatcataccttccacctagttgacccagaactgagcgccagagttaaaaggggtgtcgagaagatggatgcttcctttaaacaagtatcactttcccgtctgtaa

SEQ ID NO: 16 - DNA, 1407 bp, synthetic construct
  source 1..1407: mol_type = other DNA
atgtcatccgctcctaactatgctaagaagtgtccagttatgggcaaggccccttcaagcggacatagctccattggtcctcaggacgttcataccttagaggtactatctaatttcaatagggagaagattcccgaacgtgtagtacacgccttaggagcaggtgcctacggcgaatttgaagtgacacacgatatcagcgacatttgcaacatagacatgctacttggcgtagggaagaaaacgagccttgtcacgagattctccacgacgggcctggagcgtggctcttctgagggtgtccaagaccttaaaggtatggctacgaaattatttacgtcagacggagaatgggattgggtattcttgaattttcctttcttcttcattagggacccggtgaaatttcctgatatgatacacagccaacgtcgtgacccccagaccaataggctaaacccaaataatttttgggagttcgtaacggagaaccatgagtcaattcacatggtgctgttgcagtacagtgactttggaaggatgttcacatggaggactcttagttcatatagcgggcacgcgttcaaatgggtcatgccggatggcagcttcaagtacgtccactttttcctttcttctgatagagggcctaactttaaggatggcgccggcactattatggattcaagtgaacctgattttgcttcccgtgacttattcgaggcgatagagaggggcgaccaccctagctggactgctaacgtgcaggttattgaccctaaagatgccccgcacttagggttcaatatactggacgtaactaaacattggaatttaggaacatacccgcaaggggttgagaaaatcccaagtcgtcctttcggcaaattgaccttgaatcgtaatgtaaaagactatttctctgaggtagagcagctggctttttccccttcacacttggttcccggaatcgaggcttcagaggacccgattctacaggctaggttatttgcgtatcccgatgctcagcgttatcgtctgggtcgtactttgagtaagcaggcgtctccgcgtacaagtagcactgcagagtatcaagctagtcttgggaaagacttcgctgagtgggttgcacaagtgtcctctgacgtctggtcacagccgcatgaagacgactataagttcgcaagggaatattatgagattctaccagagtttcgtagtcaagagtttcaagataggatggtggaaagaatcgttgagtctgtatctcaaaccagacaagatataaggaacaaggtttataggaccttcgcattagtctctagtgaactagctactcgtgtacaggagggcgtcgaaggcgccgaaataggccaagaaaagaatgtgcaagctaggttataa

SEQ ID NO: 17 - DNA, 1437 bp, synthetic construct
  source 1..1437: mol_type = other DNA
atggcagccgcttccaagacatctgtagagacaaatgagagcggagactcccgttcccagagaggtaggggctaccaagcctactcagcgatcgaccaccgtcttcttgacagcctatccaggtttaatcgtgagaaaattcctgagagggctgttcatgccagaggggcaggtgcatatggagaatttgaggttaccgatgacataagtgacatatgtgacattgatatgctactgggcattggtaaaaagacgccctgtgttgtccgttttagcacaacgactttggaaagggggagcgccgaagctgtgagggatgtaaaaggaatggctatcaaactgtttactcaagatggtaattgggattgggtatgtctgaatataccgatgttctttatacgtgacccatctaaatttcctgatctgatccatgcccagcgtcccgacccgaaaactaatctaaccaatccctccaagtggtgggaatttgtatgtaacaatcatgaaacattgcacatggtgatgtttcaatttagcgactttggaacgatgttcgactatagatcaatgtcaggctacgtatctcatgcctataagtgggtcatgcccgatgggtcctggaaatatgttcactggttccttgcaagcgatcaggggcccaattttgagaaggggaagcagactaaggaaatcgcacctaacgatcccgagtcagcgacaagagacctgcatcaaagcctggatagaggtgagtaccctacttggacagtcaacgtacaagtagttgacccagaagacgctcaccaacttgcgttcaacatattagacgttacgaaacattggaacttgggcaattatccgccagatattccagtgatccccagtagaagattcgggaagctgactcttaaacaagggccaaaggactattttgaggagatagagcagttggctttttctccgagccacttagtacatggtgttgagccttcagaggaccccatgcttcaagcgagattgttcgcttatccagacgcccaaagacaccgtctgggtccccagaacctggatatgccggcaaacagaacgaggcacaccgcggatggagctaagacaaagaaggttgtaccccctgagaagaagtcgagagagcatgctgagtgggtatcacaaatgactagctccagctggagtcagccagacgaggcggactataagtttcccagggaattttggaaggcgctaccccgtttgagaggagaggagttccaaaacaacttagtggtcaatatgagcaagtccgttagccaaattcccgtggacctaaggcaaaaagtttataatacacttgggttggtagctgaagacttagccgatcgtgtcaaagctatgaccgaagaaatgatcatggaaagcgaacaaagggcaaggttataa

SEQ ID NO: 18 - DNA, 1497 bp, synthetic construct
  source 1..1497: mol_type = other DNA
atgtcatccggtgccaacgacgcaccagcgagtacatatagatatgatgaaaagcccgtttacactactagcaatggcgctccagttaacaatcctagcgggtggcagcgtccggggccaatggggccgctactattgcaagatttccatcttatcgatcagttggcccacttcgacagagaaaggatacctgagagagtcgtccacgcgaaaggcgcgggggcatacggagagtttgaagtaacgcacgacgttacagatattaccagtatcgatatgttctctaaagtaggcaagaaaaccaagtgcgtgaccagattctcaaccgtcggaggagaaaagggaagtccggattctgctagagatccgcgtggtttttccgttaagttttataccgaggaaggcaactgggactgggtgtacaacaacacacctatattctttttaagggacccaacaaactttccactgttcatacatacccagaagaggaatccccagactaatctaaaggatgcgacaatgttctgggactatctttcaactcaccaagaggctattcatcaggtcatgcatttgttttctgacagaggtactccgtacagttatcgtcacatgaatgggtattccggacatacacacaagtggactaaacccgacgggtcctttgtatatacacaagtgcatctgaaaaccgatcaggggaacaaaaccttcacgaacgaagaagcagggaagctggcttcagagaatccagactggcacacacaggacctttttgagtcaatccaaaaaggggactacccatcatggaccgtctacgttcaggtcctgacgcctgagcaggccgagaagtttcgttggaatatatttgatttgacaaaggtatggccgcaaaaagaggttccactgagaccggtagggaaactaactcttaacaaaaacgttgagaactacttcgcggagatcgagcaagttgcatttagtccctcacatctagtacctggcgtggagcccagtgcggacccggtattacaaagtcgtcttttcagctaccctgacactcatagacataggttgggcacaaattaccaacagattccagtgaatgctccgttacacccgttcaacccttatcagagagatggagcgatggtagttaacggcaattatggagctaacccaaattacccttcaagttataggccactaacctataaaacagccccacctgtgaacacgcacgaacaatggagcggctccgcagtacacgcgcttttccaggagataaatgatgacgactacgtacaagcgaaaggcttatgggatgtcctgggaaggacaagcggtcaacaagacaactttgtgggtaacgtagcgggacacctttctgctgcgcaccaagatactcgtaatagaacttatgacatgttttccagggtcgatgcgggcttaggcaaggctataaaggagcagacagaaaaacaggttaaataa

SEQ ID NO: 19 - DNA, 1443 bp, synthetic construct
  source 1..1443: mol_type = other DNA
atgtcatccgccccgaacgcggccgataagtgcccggtaatgggtaattcaggtgaaaaatgtcctgtgatgtcatccagtactcagtccagaggcccgcgtgatatttatacgctagaagcgttatctcatttcaaccgtgagaagataccagaaagggcagttcacgcgaaaggaactggggcctacggcgagttcgaagtgacagctgacattagtgatatatgcaatatagatatgctgctgggagtcggtaagaaaacacaatgcgttactcgtttttctacaaccggacttgagagggggtccagcgacggtgtgagagatttaaaaggcatggcggtcaagttctttacagagcagggggactgggactgggtctccttgaattttccgttctttttcattagggaccctgctaaatttcctgatatgatacacagccagagacgtgatccgcagactaacttactgaatccgaacatgacttgggattttgtgacgaagaatcctgaggcgttacacatgaccctattgcaacattccgacttcggaactatgttcacttggcgtaccctaagctcctacgtgggacacgctttcaagtgggtaatgccggatggctctttcaaatacgtgcacttctttcttgcaagtgacagaggtccgaactttacggatggtagtactgccaaagtcgatcctaatgacccggactttgcgaccaaggatttgttcgaggccattgaacgtggcgactatccgagttggacagctaatgtccaagtagtggacccaaaagacgcacctaaacttggctttaacatcttagatctaacaaaacactggaatttagggacatatccaaagggattggacaccatcccgagccgtcctttcggcaagttaacgcttaatcgtaacgttaaagactatttcagtgaagtagaaaaactagctttctctcccagcaacctagtgccaggagttgagccttctgaggacccaatattgcaggctagaatgtttgcttatccggatgcacaaaggtataggttaggtattgatcatttaaaggcaccacttagaagaaaagaaacagcttgccagcacgatcttggaccggagttcgaaaagtggttgagtcaggtgacctccgaggcgtggtcccacccacatgaagacgattacaaatttgcgagggaatactatgaagtcctgccggaatttaggagccaggagtttcaggacaggatggtagaaaacctgtgtaaaagtatcgcacccggcccagaggaactgaggaagagagtttatgatacctttgagcttgtgagcagtgaattagcaaggagacttagggagggtgcagaagcaatcgtcgccgaaaaggcaaggcccgactccccttcccgtgctcaaccgggtcagttgagattgtaa

SEQ ID NO: 20 - DNA, 792 bp, synthetic construct
  source 1..792: mol_type = other DNA
atgtcatccgcttccgttgaaagcagaatcattgctatcacaggtggagcatccgggataggcgcggccacttgtagattacttgctgagaggggtgctgccgtactttgtgtgtgcgacatatcccccaaaaattttgacgacttgaaaatttccataaagaagatcaacccctctaccaaggtccactgtgctacggtggatgtaacatcatccgtggaagtgagacagtggattgagggaatcatatctgactttggcgatttgcacggtgccgtgaatgctgctgggatcgctcagggagccggtatgcgtaacacccctactattgcggaggaagtcgatgaggagtggacccgtattatgaataccaatctaaatggggtgttctactgcacgagggaagaggtacgtgcaatgaagggattacccgctacagataggagcattgtcaatgtcggaagtatcgcatccgtctcacatatgcccgatgtgtatgcctacggtacctcaaagggagcctgtgcgtattttaccacgtgtgtggcggcagacgcatttccccttggaataagaatcaataacgtgagcccaggggtgacgaatacacctatgttaccgcagtttgcgcctatggcgaaaacttttgaggaaatcgaagagtcttataagaaagaaggtttaagtctaattgaagccgaggatgtggcaaggacaattgtctggctactgtctgaagattcacgtcccgtgttcggagcaaatatcaatgtaggtgcgtgtatgccataa

SEQ ID NO: 21 - DNA, 792 bp, synthetic construct
  source 1..792: mol_type = other DNA
atgtcatccacagtagtaaattcaaggatattcgcagtaaccggcggtgcttccggtattggggccgcgacttgccgtcttctggcggcccgtggggcggctgtcatctgcataggcgatatatcaccgaagggattcgagagtgtaacatctgacattcaagcactgaatccggccacgaaagtccattgcacaagactagatgtcagttcatcagcggaggtcgacgcctggcttcaaactatcgtccagacctttggcgacctgcatggactggctaacgttgcaggtatcgcacaaccggctggagcgagacaggcgcctgcccttttagaagaaacagacgaagagtggcaaagagtattcaaagtgaatctagatggtgtgttttattgcacgagggcagggataagggcaatgaaggcattgccaggtccaaaggaccgtgctattgtcaacattggctctatcgccgccttcatacacctacctgatgcatatgcctatgggacctccaaaggcgcctgtgcctatttcactagttgtgctgccatggatgcttggccgttgggtattcgtatcaatagcgtgagtccgggcataactgacactcctgcgttgccgcaatttatgcctcaggccaagtctatagacgaggtcaaagaggcctacatcaaggagggctttccgataattcagccggaggacgtagctagaactattgtttggttacttagtgaagatagcaggcctgtttacggcgcaaacataaacgttggggctgccacgccc

SEQ ID NO: 22 - DNA, 789 bp, synthetic construct
  source 1..789: mol_type = other DNA
atgtcatccgcctctgtttcctcaggaatttttgcagtaactggtggagccagcggtatcggcgcggcgacttgtaggcttttagcagacaggggcgcagccattatttgtttggcagatgtctctaacaccaatttcgtgtcactacagcgtgagatcgcagaaatcaatccgtctacctttacacaatgtacggtcctggatgttactagcccccaggatgtcaacaagtggctgcaggatataatctcaacacacggggatctacatggggccgctaacgtagctggtatagcgcagggcgcgggtctaagagcttctcccacgatactggaagaaaatgattctgagtggagaagaatactggacgtaaatttaaacggcatattatactccactagagcacaggttagagtcatgaaggaattgactcacggcaaccgtagtatagtcaatgttgcgtcaatcgcagcatttagtcatgtcccggacgtgtatgcttacagcaccagcaaaaatgcttgtgcttacctaactacctgcatcgctgctgacgttgtctcactgggcatacgtgttaactgcgtttcacctggtattaccaacactccacttttgccacaatttgaaccgaaggcaaaatctctttcagagatccaggaaatatatttaagacaaggacttcctattcacgaagcagaagatgttgcccgtacaatcgtttggttgcttagtgacgatagtaagccggtgtatggcgccaatatcaacgtgggtgcatgcgcaccg

SEQ ID NO: 23 - DNA, 792 bp, synthetic construct
  source 1..792: mol_type = other DNA
atgtcatccccgagtatgacttcaaaggtatttgcgataacaggtggggcgagtggcattggcgcagccacctgtagactgcttgcggagagggatgccgctgtgatttgtttagccgacgtatccagtacaaattttaccagtcttcaggagtcaatagcaaaaagcaatccgagtaccctggtacattgtactgaattggatgtccgtagcgccgataaggtagatcagtggctacagtccattgtgagcacgcatggagatcttcatggtgctgccaacgtggcgggtatagctcagggggcaggactgagagcgacaccgacgattctggaggagaacgatgccgagtggtcaagaatcctggacgttaaccttaatggtgtattctattccacgagggctcaagtgagagtgatgaaggatttacctccaggtcaccgtagtatagtcaatgtggcttctatcgccgctttttcacacgtcccagatgtttatgcttatgggactagcaagtccgcatgtgcatacttgacgacctgtattgcagcagatgtattctggtcaggtatacgtgtgaactgcgtctcaccaggaatcactaacactcccatgcttccgcagttcgagcctaaggcaaagtcacttgatgcgattaaggacatgtaccgtgaccaagggtaccctacgggtgaagcggatggagtagctaggaccatagtatggctgctaagtgaggattctatacctgtctatggtgcgaacatcaacgtcggggcctgcccgccataa

SEQ ID NO: 24 - DNA, 792 bp, synthetic construct
  source 1..792: mol_type = other DNA
atggcagccagttcagtgtctagcaaaatcttcgccataacaggaggggcttccggcatcggcgctgcaacatgcagattgttggcagacagaggggcagctgtgatatgtcttgctgatgtcagtagcaacaattttgtctctctgcaagaaagcatagccaaggttaattcaagtactcttgttcaatgtaccgctttagatgtaagcatggcaaatgaagtaaaccaatggttgcaggacatcatttcaatacatggcgacctacacggagcggcgaatgtagctggaatagcacaaggggcgggattgagggctaggccaacgatccttgaggaaaaggatgacgattggtcaagaatattagacgtgaatctgaacggtgtgttctactctactcgtgctcaggtacgtgcgatgaaggatctgccccgtgggcaccgttctatcgttaatgtcgctagtatatctgtcttctcccatgtaccagatgtttacgcatatagcacttctaaaaatgcgtgtgcttatttgactacctgtatagcggctgatgtcttctggtctggcatcagagtcaattgcgtttcacccggcattacaaatactccgatgttgccagatttcgagccaaaggcaaagtcactggacgaaatctacgagatatatagaggtcaaggctacttgacatgcgaggccgaggatgtggccagaacaattgtctggttactatccgaggactccagaccggtatatggcgcgaatatcaatgtaggcgcctgtccaccttaa

SEQ ID NO: 25 - DNA, 789 bp, synthetic construct
  source 1..789: mol_type = other DNA
atgtcatccacctcagttacatctaaagtatttgccatcacgggaggggcctcaggcataggggctgcaacgtgcaggttgctttccaagaggggcgcgagtgtcatttgtgtgggtgacatagtgaacgaccacttcgacgaacttcgtagggacatagcacgtatcaataacacgacgaaggtaagttgcacggtgcttgatgtaacttcaagcgctcagatcgaaagatggatctcttatatcgttgacacatacggtgacctttatggggccgcgaatatcgcgggtatagcgcagggtgcggacctaaggacctcccccaccatccttgaggagaccgatgatgagtggtcaaaagtaatgaaagttaatcttgacggggtgttctactgcaccagagctgagatcaggggcatgaaaaatttggagcgtattgggagaagtatagtcaatgttgggtcaatcgccgcctttcttcaccagccggacgtgtacgcttacggcacgtccaaagctgcctgcgcttatatgtccacttgcgccgctgctgacgccttccccttcagaattagagtcaacaccgtatcccccggtattactaataccccgttgttaccatcatttaatccgaaattgaacagcttggatgaggtgaaagagttatatgcaaagaatggcttaccactgatagaacctgaagacgtggcgaggactattgtctggttattatcagacgattcctggcccgtctacggcagtcatatcaacgtcggggcgtgcgtgcct

SEQ ID NO: 26 - DNA, 792 bp, synthetic construct
  source 1..792: mol_type = other DNA
atgtcatccgcatctttaactgccaaaatctttgcggtcactggtggtgcatcaggcataggagcggccacgtgcaggctgttggccgagcgtggggcaggggtcatctgtgtagccgatgtatctagtacgaattttggcagcctacagaagtccatcacagaaattaactcccttaccctagtgcaatgtacggtacttgacgtgacttctagcgaggatgtgaatcaatggcttagggacatcgtaagttctcatgggaacctgcatggtgccgcgaacatcgccggggtcgcgcagggggctggtttacgtaccacgcctacaatcctggcggagacagacgccgattggtcaaggatacttaaagtaaacctagacggcgtattttattgcacgagagcccaggtgagagcgatgaccgacctaccaaaaggtaacaggtctattgtcaacgttgcatctattgctgcattttcccacatgcccgatgtctatgcttacagcacttcaaagcaggcgtgtgttcatttcacagcgtgcgtcgccgccgacgtgttccctctgggaataagggttaattgcgtcagcccaggcattactaataccccattattaccgcaatttgaaccgaatgctaaaagtttagacgaaatacaggctctttacgcaagtcaagggttgacaatgtgtggtgctgatgaggtagccaggacaatagtgtggctgctgtccgaggatagtcacccagtgtacggcgctaatataaatgtcggagcgggagtgccctaa

SEQ ID NO: 27 - DNA, 792 bp, synthetic construct
  source 1..792: mol_type = other DNA
atgtcatccgcatccgtgaaatcacgtgtcttcgctatcacgggtggagctagtggtataggagcagcgaccagcagattgctggcggagcgtggtgccactgctgtctgtgtcggtgatataagttgtaagaatttcgatgaactaaaagagtccatgaagaaaatcaatcctgccacagaggtacattgctccctactagacgtgacgagccccaccgaggttgagaaatgggttaaaagcatcgtggcgaaatttggcaaccttcatggggcggcgaacatagcgggcatagcacagggtgcgggccttaggaacagccccacgatccttgaagaaggagacgaggagtggtccaaggtgctgaaggtgaacttggacggggtcttttattgcacaagagcggaggttagagcaatgaagagcctgccgtcagacgataggtctatcgtcaatgtgggctctatagctgcgctgtcacatattcccgacgtatatgcgtatggaaccagtaaaggggcttccacgtacttcacaacatgcgtcgccgcagacaccttcccgtttggcatcagggtcaattcagtctccccaggaattaccgatactcccctgctgccccagtttattcctaaagcaaagacgagtgatgaagtgaaagaagtttatcgtaaggaaggtttcagcgttgtcaaggcaggtgacgtcgcaagaaccatcgtgtggctgctgtctgaggatagttcacccgtgtatggagctaacataaacgtcggggccagtatgccgtaa

SEQ ID NO: 28 - DNA, 792 bp, synthetic construct
  source 1..792: mol_type = other DNA
atggcagcctctagcgtctctagcaagatattcgccataacagggggtgctagtgggatcggggctgcaacgtgcaggcttctggcggatcgtggtgcagcgatcatttgtctggcagacgtaagcagcaataacttcgtctcccttcaggaatccattgcagaggttaatccttctacactggttcagtgtaccgtcttggatgtaagctctgcagtcgatgtcaaccaatggttacaagatattgtttccatccacggtgacttacacggcgccgccaatgttgcagggatagcccaaggcgcggggctgagggctactccaactatactggaggaaaaagatgccgattggagtaggatattggatgtgaatttaaacggagttttctatagtacaagagcccaagtcagggcgatgaaaaacctgccgtgcggtaataggagcatagtcaatgtggcaagtataagtgctttctctcacgtgccagacgtatatgcatattccacgtcaaagaacgcttgtgcgtatttaacgacttgcatcgccgccgacgtattctggtcaggtatcagagtaaactgcgtcagtccgggtatcacgaatactccattgcttccgcagtttgagccaaaagctaagtctcttaatgaaattcaagaaatttacagaagacaggggtactcaacgtgtgaagctgaagatgtagcgaggaccattgtatggctgctatctgatgattctaggcctgtgtatggggctaatataaacgtgggggcttgcccaccttaa

SEQ ID NO: 29 - DNA, 804 bp, synthetic construct
  source 1..804: mol_type = other DNA
atgtcatcctcatttgtaagcagcaaaatcttcgccataactggcggtgcatcagggattggagcagcgacttgtaggcttttagcgaagagaggagcggcaacactgtgcgtaggcgacttatgtagtgagaatatgaaactattggaaaaagacattaaaaagatcaaccctgacaccaaagtacactgtaccgtcctagatgtgagttccagcagtaatgtggatgagtggatacaggatattattaccacttttggtgacctacacggtgctgcgaacatagccggtattgctcaaggtgctggcctacgtcaggctccgactatattagaagacgacgatcagcaatggaagaaagtcttccaggttaacttagatggcgttttgtattcaactagggcgcaagtccgtgccatgaaagagttcagctcaacgaatccgggcgacagaagtatcgttaacgtcgcctccatcgcctccatgagtcacatgccggatgtttttgcatacggcaccagtaaggctgggtgcgcgtacttcactactttcgaggtcacattgttcgcattagggtacttctctaacataatgggaatgctagaagggattacgcgtactccaatgcttcccagattcgttccatcagctaaaactcaagaggaagtcgaggagacctacaagaaggagggattctcagtaattgaagcagacgacgttgcaaggacgattgtatggttactgagcgaggactcacgtccagtgtttggggcaaatattaacgtcggagcatgtatgccgtaa

SEQ ID NO: 30 - DNA, 1137 bp, synthetic construct
  source 1..1137: mol_type = other DNA
atgtcatccagagaggagccaagttccgcgcaattatttaaacctttgaaggtcggaaggtgtcatcttcagcatcgtatgattatggctcccacaaccaggtttagggccgacgggcaaggggttccccttccatttgtgcaagagtactacggacagagggcctcagtcccaggcacgctattgataacagaagctaccgacatcactcctaaagcaatgggatataagcatgtccctggtatctggtccgagcctcagcgtgaagcgtggagggagatcgtgagcagggtccatagcaagaaatgctttatattttgccagttatgggccacaggcagagcggcagacccagacgttctagcggacatgaaagacttaatatcttcctccgctgtacccgtggaagaaaagggtccattaccacgtgcattgaccgaggacgaaatacaacagtgcatagcagactttgcccaagccgcaaggaatgccattaacgccggttttgacggtgtagaaatccacggagctaatgggtacttgatagatcagtttacgcagaaatcttgtaaccaccgtcaagaccgttggggtggtagtatcgagaatagggcaaggtttgcggtggaagtaactcgtgccgtaatcgaagcggtcggagcggatagagtcggtgttaagttaagcccgtacagccaatatcttggaatggggactatggatgagctagtgcctcaattcgagtacttgatcgcccagatgcgtaggctagacgtcgcttacctgcatctggcaaactctcgttggttggatgaagagaagccgcatccagatccgaaccatgaggtattcgttagagtctggggacaaagctcccccatcttgttggcgggaggctacgatgctgctagtgcggaaaaggtgactgaacaaatggcggcagcgacctatacaaacgttgcaatcgcattcggtcgttactttatatcaacccccgacttaccttttcgtgtgatggccggaatacaactgcaaaagtacgatcgtgcctccttttactccacactaagtagggagggttaccttgattacccgttcagtgccgaatacatggctcttcataacttccccgtataa

SEQ ID NO: 31 - DNA, 1131 bp, synthetic construct
  source 1..1131: mol_type = other DNA
atgtcatccagcacgtcaaaacttttcaaagcgtttcctttaggaaccaatgtgttacagcataaagtcgtgttgtcacctatgaccagatttagagcggatgagaacggggtgccgttgccttatgtgaaaacgttttacgctcagcgtgcttgcatcccaggcacgttattaatcaccgaggcggttgccatctccccaagagccaaagggttccagaatgtcccgggcatttggcaccccgatcaggtgtctgggtggcgtgacgtggtagacgaggttcatagtaaaggtgcagtcatctggctacaactttgggccacagggcgtgcagcagatcaggcggcgctggcttccaatgggttagagcttgagtcaagtagtgaggtacccgtcgcccctggggaacccacgccacgtgctttgagcgaggaggatattgagtcatatatcttcgactatgtccagggcgcgaaaaatgcgatccatggtgctggtttcgatggtgtagaaattcacggggcgaacggatttttaatagatcagtttctacagagtagttgtaacaggcgtaccgaccagtggggaggcagtatagaaaatagaagccgttttgggctggagataaccagacaggtaataaatgctgtcgggaaggatagggtcggtatgaagctatccccttggtccacattccaagggatgggtacgatggatgaccttgtgccgcaatttcaaaatttcatagcggagcttagagaaatgggtatcgcttacctacatcttgcgaactcaaggtgggtggaagaggaagaccctaccataaagacccaccctgacgtaaacaacgagaccttcgtcagaatgtggggagagaagcgtcctattctgttggctggcgggtatggacccgactcagcaaggcacgtcgtagaccagacgtatgcggaacagaataacgttttggttgtcttcggacgtcattatataagtaatccggacctgccttttagattgaaaatggggatagcacttcaaaagtataaccgtgacacgttttatatcccggcttctgataaaggctacttagattacccattttgtgaggagtacctgaagtacgtcggtaag

SEQ ID NO: 32 - DNA, 1149 bp, synthetic construct
  source 1..1149: mol_type = other DNA
atgtcatcctcaacctctaatttgtttagtacagtaccttttgggaaaaatgtcctaaatcacaagatcgttttaagcccaatgacgagattcagggcagacgataatggggttccgttatcctacatgaaaactttttacgctcaaagagcgtcagtcaggggcacccttttggttaccgatgcagtcgctatctgccctcgtacgaagggtttccctaatgtaccgggtatatggcataaagaccgtatagctgcctggaaagaggtggttgacgaagttcattcaaagggctcctttatctggttacaattgtgggcgacagggcgtgcagcagatttggaggcattaacctctagggggttaaagttgggctcaagctctgaggttcctgtagcgcccggtgaacccacccctagagcccttgatgaagatgagatccaacagtacatcctggattatgtccagggtgctaagaacgctgtacatggcgcagggttcgatggggtagagatccacggagccaatggtttccttatcgatcagttcctgcaatcctcatgcaataggcgtaccgaccagtggggtggttcaatcgagaataggtctaggtttgggctagaaataacaaggggcgtcgttgatgcagttggacatgatagggtgggtatgaagttgagcccgtggagtacgttccaaggtatggggactatggacgatctggtgccacagtttgaacatttcattacatgcctgcgtgagatggatatagcgtatttgcatcttgcaaactccaggtgggttgaggaggaggacccctcaattcgtacccatccagatttccataaccagactttcgtgcaaatgtggggcaagaagaggccgattctactagccggagggtatgatcctgacagtgctaggaggttagtggatcagacgtatagcgatcgtaacaatgtgttggtggtatttgggcgtcattatatctctaatcccgaccttccctttagactgagaatgggtatcgccttacaaaaatataatcgtgatacgttctatataccctgttccggagagggttatgttgactatcccttctgcaaagagtaccttgaccaagccgacgaagctgcagtggccgggtaa

SEQ ID NO: 33 - DNA, 1161 bp, synthetic construct
  source 1..1161: mol_type = other DNA
atgtcatccagcacaagtaaccttttcaacgctcttccttttggaagaaatgttttagagcataagattgtgttatcaccgatgacacgttttagagctgataatagcggggttccgttaccttacgttaaaacgtactacgcacaaagagcgagcgtaagaggaacccttcttatcacggaggcggtcgcgatatgcccgcgtgctaagggatttcctaatattccaggcatttggaacgactcacagattgctgcctggaaagaagtagttgatgaagtgcattccaagggtagtgttatctggttacagctgtgggccacagggagagcggccgacactgataccttgactagcaacggttgggagctagagtcaagttccgaggtacctgtcgccccgggcgggccgactcctagaagtttgactgaggaggaaatacaatcctacatagtcgattacgcgcaagcggcgagaaacgctgttcacgaagcgggctttgacggagtagagattcacggggctaatggctttcttgttgaccagtttttgcaatcttcttgcaataggcgtactgatcagtggggcggatctattgaaaatcgtagtcgttttggactagaaataaccaggagggttatcgacgccgtgggcaacgacagggttggcgtgaagctatctccttggtccacctttcaaggcatggggacgatgggggacctagtgttacaattcgaacactttatcacaaacctgagagaaatggatattgcctatcttcatttagcaaattctcgatggatagaggaagaggacccgagcattgagacgcatccagatatacacaacgagaccttcgttcacatctggggtgaaaagagacctattttattagctggcgggtatgatagtgactctgctcaaagattggtggaccagacgtactcccagcagaataatgttatggtagtttttggaaggcactacatttcaaacccagatttaccattccgtctgaagatgggcatcgcgctacaaaggtataatcgtgattcattctacataaaattcagcggagaaggctacctggattaccccttctgtaaagagtacctggaacaggaggatgaggacaccactgcctctatgaggaagaattaa

SEQ ID NO: 34 - DNA, 1107 bp, synthetic construct
  source 1..1107: mol_type = other DNA
atgtcatcctctaaccagagtggtctattttgccccttacagcttgggaagtgttatcttaaaaacagggtgatcttgtgtccgcttacccgttttcgtgcggacgtcggtgctgtcccctctagcctgatgaaggaatactatacgcagagggctagtgtccccggcacactgttaataactgaggcaacgtcaatcagcgaagacgctcgtggcttcgacaaggttccgggtatttggaatcaggatcagattgcggcatggaaaacaattgtcgacgcggtccatagtaaaaattccttcatatggctacaactttgggcgaccggacgtgcagccgagcctggtttcaattgcgtctcctcatcatccattcaaatcccaccacaagaggacccgaacgagatctatcccgtgcctcgttccctgacgtccatggaggttgaaaatttcataaatcagtacgtcttggctgccgagaacgctatgaattgcggttttgacggtgtcgagattcatggcgccaatggatttttgattgatcagttcacgcaggaatcctgtaacaagcgtacagacgaatggggaggctccttggaaaagagagctagattcggactggaggtcacgaagcgtatcatatcaaaaataggggcagacagggtcggtataaagttatcaccatggtctacatacttaggcatgggagtgatggaagacttagtaccccagttcgagtatataatcaaacagctgcatgaaatgaatgtggcctatttacatctggccaattcacgttgggtcgagagcaaatctcatccggatgtggacaatgagacgttcgtaagaatgtggggcaaggcgaagcctataattattgctggaggatataatggcgagtcagctaggaaagtggtagaagatacgtatgccgggcataataatatagccgtagggttcggacgtcacttcttagcaaaccctgatttgccctttaggcttaggcacggtatccaactagctccttacgaccgtgagactttctacggtcccggatccgagggatacacagactaccctttctctagcgagtttatagattcacac

SEQ ID NO: 35 - DNA, 1140 bp, synthetic construct
  source 1..1140: mol_type = other DNA
atgtcatcctccacctcaaaccttttcaagagtatagcatttggaaaaggggtgctgcagcacaagatcgtcctttcccccatgacaaggttcagggcagacgacgacggagtacccctaccatatgtgaagacctactactcacaaagagcttgcttacctggaactcttttaatcactgaggcagtagcaatttcaccgagggcaaaggggtttccaaatgtaccagggatttggtcacccgaacagattgcaagttggaaagaagtagttgatgcggtacactccaagggctcatatatatggttgcagttatgggccactggcagagcagcagatgtagatacacttaaagcgaatggtttcgaaatagtaagttcatccgcaactcctgtcgggcccgacgaaccaagcccgagggctctgaccgaagatgagatcacatcttacatcgccgactacgttcagggtgcaaaaaacgccgttttcggagctggttttgacggtgttgaaatccatggagcaaatggttttttaatcgaccagtttctgcaaagttcctgtaaccagagaacggacggttggggtggatcaattgaaaaacgttcaagatttgggttggaaataacgaagcaagttatagacgcgatagggagagatagggttggtatgaagttatcaacttggtctacgttccaagggatggggaccatggaagatcttgttccccagtttcattacttcctaagccacctagctgagatggatatcgcttatgttcacctggcgaatagccgttgggttgaagaggaagatcctaccataaggacccatccagacatacataacgagacctttgtaaggatgtggggagacaaaaaaccgatcttattagcgggaggttacgatgccgagtcagctcagagagtcgtagatgagacatacgctaaacaagacaacgtgctagtggtattcggccgtcattacatctctaatcccgaccttccattcagactgaaaatgtctattgacttgcaaaagtataatagggacactttctatatacccttcagcgatgaaggatacctggattatccttacagcgaggagtatcttgcacaagaacagggagctacagca

SEQ ID NO: 36 - DNA, 1146 bp, synthetic construct
  source 1..1146: mol_type = other DNA
atgtcatcctccacctccaatctatttacaccactacagttcggaaaatgccttttacaacacaagctagttctgtctcctatgacgagatttagagcagacaatgagggagtcccattgccctatgtgaagacctattactgtcagagggctagcttacccggtacacttctactaaccgaagcaaccgctatcagcagaagagcaagggggtttcccaatgtgcccgggatctggtcccaagagcagatagcgggatggaaggaagtagttgacgcggtccatgctaaggggagttacatttggttgcaactttgggcaactgggagggccgcggaagtgggagtccttaaagctaatgggtttgaccttgtcagctcttcagctgtgccggtgtctcccggagagccgaccccgcgtgctcttagcgacgatgaaatcaatagctacataggagactttgtgcaggcggcaaagaacgcggttttggaggccggcttcgatggcgtggaattacatggagctaatggcttcctaattgaccaatttttacaatccccatgcaaccagaggacagaccaatggggtggctgcattgaaaacaggtccagatttggtttggaaattaccagaagagtgatcgacgcggtagggaaggatcacgttggcatgaaattgtccacatggagcaccttccagggtatgggaacaatggatgatctaatccctcaatttgagcacttcattatgaggcttagggaaatcggcatcgcctacttgcaccttgctaatagcaggtgggtagaagaagaagatccgactatacgtacccaccccgacatacacaatgaaacattcgttaggatgtggggcaaggaaaagcccgtgcttctggctgggggatacggaccagagtctgcgaaacttgttgtggatgagacatacagcgaccacaagaacataggggtcgtattcggaagacactacatatctaaccctgacttaccctttagacttaaaatgggtcttccactgcaaaaatataatcgtgaaacgttctatatacctttcagtgatgaagggtatcttgattacccatactctgaagagtatataactgagaacaaaaaacaagccgtactagct

SEQ ID NO: 37 - DNA, 1134 bp, synthetic construct
  source 1..1134: mol_type = other DNA
atgtcatccactatcattcctaaacatccttctcctactttatttaagcccttagcgttaggtaaatgccaattgcagcatcgtatcgtcatgagtcctactacccgttatagggcggatgaggcggcagtcccattgcccttcgtcaaagagtattatgctcagagggccagtgacccgggggccttgcttattacagaggctaccaacatatgccctaactccgttggggaagctcatatacctggcatctggtctaaaacacaatgtgaagcgtggagagaagttgtgagtcaagtgcatgcaaaagagtgttacatcttctgtcaaatctatgccacgggaaggagtgctgatccagaattacttgcctctaggggatttgagcaagtctcaagctcagcggtggcagcggaacctggatgccagccgcctcgtgccctggacgaggaagagatacaaaagtacatctccgactatgcccaggcggctagaaatgccatagaggtcggatttgacggggtagaaatacacggtgcaaacggatacttgattgaccagttcacccaagctagttgtaatcagcgtacggacgagtggggaggtgacattccaaatcgtgcaaggtttgcattgcaagtcactatggcggtaatcaatgccattggcccggatagggttggcatgaagctaagtccgtggagccagtattcagggatgggtataatgggagacttggttccgcaattcgagtatctgatattacaactaagacagctaggtattgcatacctgcacttagcaaattcccgttggcttgaccaaatgactactcatccagatccaaatcatttgactttcgttaaggtatggggaaggtcattgccggtgatacttgcgggcgggtatgacgcgacttccgctcctgaagttatagagatggtttacgccgattatgacaatgttgccataggatttggtagatactttacaagtacacctgatctacccttcaggatgaagaacgggatcgcccttcagaagtatgatagaagtagcttttacacctgtctgactaagactggttatttggactacccatattctcccgaatacctttgcagatcctcataa

SEQ ID NO: 38 - DNA, 1149 bp, synthetic construct
  source 1..1149: mol_type = other DNA
atggcagcctccacgtccaacctattcaatgaattaccattcggaacctctgtcctaaaacacaaaatagtcttatctcctatgaccaggttccgtgccgataacggaggcgtccctttaccgtatgttaaaacttattatgcccaaagggcgtccgtgcatggcactctgctgattaccgaagcagtggctatctgtcccagggcgaaggggttcccgaatatccctggtatatggcacgacagccaaatcgccgcttggaaaggggtggtggacgaagtacattccaaaggttccgtaatctggctacagctttgggccaccggcagagcggtagaccctgacacattaacgtcaaacggattagagttgaaaagctcctcagaagttccagttgctcccggggaacctacacctaggcctctaagtgaggaggagattcagagctacatttttgactacgcacagggcgcgaaaaacgccgtccacgaggcggggtttgatggagtagagatccacggggcaaacggctttttaatcgaccagtttttgcagagttcatgtaatagaaggacggatcaatggggtgggtctatagagaatagatcaagattcggacttgagataacaagaagggttattgatgcggtgggaaacgatcacgtgggcgttaaattgagtccttggtcaacgtttcagggaatgggaacaatggatgatctggtcccacagtttgaacacttcattacgagactaagggaaatggacatagcttatcttcacctagccaattcccgttgggtggaggaggaagacccatcaataaaaacccacccagacgtacacaatgaaacatttgttagaatctggggagaaaagaggccgatactgctggccggaggttacaatccagattcagcaagacgtttggtggaccaaacatatagcgaacaaaacaacgtaatggtggtatttggtcgtcattatatctcaaacccagacctgccgtttagactgaagatggggattgcgttgcagagatacaacagagataccttttacatccctttcagtggggaaggatatcttgattacccattctgcaaagaatatctgaaacagggtgaccaagaaacaactgttgcttaa

SEQ ID NO: 39 - DNA, 858 bp, synthetic construct
  source 1..858: mol_type = other DNA
atgtcatccacaatcctggttttaggcggcagaggtaaaactgctagtagactatccgcgctgctgcatgaggctcagataccgtttgtggtaggcacgagctcaaccgccccgatagccacatatagaaccgcgcactttgactggttaaataaagatacctggacgaacgcctgggaccaagcaccagagccgataacagcaatctacttggttggggcccacgccccggagttcgtccacccgatgttcgagtttatcgatttagcacacagccgtggcgtcagaagatatgccctagtcagcgcgagcaatactgagaaaggtgacccaatgatgggcgaagcccatgcacaccttgactcaaaagagggggtcaaatatgtcgtccttaggcctacttggttcatggaaaaccacatagaagatcctcatctaagctggataaagacggagaacaagatctatagtgcaacggggcccggaaagatacctttcgtgagcgcggacgacatcgcgagagttgcttttcacgctctaacacaaccgcttacccaaaaggaacaacagtacatggtcctggggccagaccttttgagttacgctgatatggcagaaatccttaccgacttgctgggtaggaagataacccatgttacactgcctgccgaaaagttcggacagtttctggtagataccgcaggcttgccgccagattttgctgaaatgctagccacgatggaggcagccgtagcaaccggggccgaagagagagtgtcccaggacgtagagaaagtgacaggccgtcctcctagacgtttcgttgactacgctagggaaaatagggagcactggttg

SEQ ID NO: 40 - DNA, 870 bp, synthetic construct
  source 1..870: mol_type = other DNA
atgtcatccacaatcttactgactggcggctctggtaagaccgcatcaagaattgcccacttactaaagaaggcgaacctgcctttcgtcatcggaagcaggtccagcagcccacagaccgaaaaataccatcgttcttttaattggctggatgagactacgttccagaatgtgctttcatcagagaggggtacgcagcctatttctgccgtctggttagtgcctccaccgatactggaccttgcaccgccggttaacaatttcatcgattacgcttgctcccaaggcgtaaaaagatacgtactgttaagcgcatctatgattgaaaagggcggcccggcgatggggcagatccatgagcacttagattctcttgaatccatctcctacacagtattacgtccaacctggtttatggagaactttagtacacctggagaattccagtgtgaatctattagaaatgaagcgaaagtctattccgcagccggagatgggaaagttccgttcatctccgcagcagatatagcccgtgtagcatttcgtgctttgacgggaaagttgcaggaccgtacggaccatattttgctaggtccagaactgttggcgtacgacgatgtggcggagaccctttctaaggtgatcaacaggaaaataacccatgctagggttactgaaagtgagttgtcagaaaaactacaagcaagaggtatgtccgcagaggaggcagagatgcactctagccttgaccttacgatcaaaagcggggccgccgcggacatgaacaccgtcgtaagagacttaacaggctccgaaccgtgtaatttcctggagttcgcaaagcgtgaaaaaaaggtttggttgtcaaac

SEQ ID NO: 41 - DNA, 879 bp, synthetic construct
  source 1..879: mol_type = other DNA
atgtcatccacagtgctacttacgggagggactggaagaacagcgaaacatatcgcagggatatttcgtcaaaccaatgtaccatttttggtggcctcccgtagctcctccgcaggcacagcggagaaccacaggaaatttgattggctggatgaggagacgttcccgaacgctcttagtgtggaccagggcatgaagccgatatcagtagtgtggttgtgcccgcccccgttgtacgatttggcaacccccgtgattaagttcattgatttcgcagtaagccagaatgtcaagaagtttgtccttctgagcgcctctgtcatccagaagggtggccctgcaatgggaaaaattcacggccacttagattctatcaaagacgttacatacacagtgctaaggccgacatggttcatggagaacttcagcacaaaaggtgaaattcaatgtgaagctattcgtagggattccaccgtttactcagctaccgagaacggtaaaatcccctttatctcagtagtagacatcgcacgtgtggcagcttgcgcgttgacggctgagacattaaagaacagcgaccacatacttcaagggccggatttgttaacatacgatgaggtagcacaagcccttaccggggtactagggagaaaaattacacataccaagatgacagagggggagttggctgaaaagttgatggaagagggtgttaccccagaagaggcatacatgcatgctgcgatggactcaatgatcaaaagcggaagcgaggaaagggtggtttcagacgaagttaaggagtggaccggcgttaagccacgtggctttatcaactttgcgttgtcagaaaaggctgcgtggcgtgctagaaagtaa

SEQ ID NO: 42 - DNA, 876 bp, synthetic construct
  source 1..876: mol_type = other DNA
atgtcatccaccatactacttacaggaggaaatggaaagacagcaaggcatatagccagtttgttagaagaagcaaacttgccgttcgtaattggcagccgttctagcagaccgcaaaacattggtagacatagatcatttgactggcttgacgagaagacttttcaaaacatcttgactcctggggaggggatggagcctatctcagcagtatggctggttcccccgccaatctctgatctagcatccccggtaaataagttcatcgactttgcatgctccaaagatgtcacaagattcgtgttactatctgctagtatcgtagaaaaaggtggacccgcgatgggtcaaatccatgcgcacctagatagtttagaaggaataagctataccgtgctaaggccaacttggtttatggagaactttagtaccaagggggactttcagtgtgacaccatcaggaaggaagatacgatctattccgctaccggggacggaaagattccctttattagcgtcgtagatattgcgagagtggccttgagagcccttaccgctaacgttcctgaccagatggatcatgtcttacttgggccagaattgcttacatatgatgacgttgcagaaacgcttactcgtattttggataggaaaattacccacgcgaggctgaccgaatccgagttagtgcagaaaatgcagaagtccgggatgagtgccgaggatgcccagatgcacgcttctatggatctgatagtacagagtggagctgaagaccgtttaaatactgtagttagggagttgacgggctcagaacctaggaacttttctgacttcgcgttaaatgaagctaagctgtgggtcagcagaaattaa

SEQ ID NO: 43 - DNA, 870 bp, synthetic construct
  source 1..870: mol_type = other DNA
atgtcatccacaacgctactactgggaggtcatggtaaaaccgcacttcaaatagctgctattcttagagcgtctaaccaaagcttcctagtagcgtctcgtaccgcgagtgcaggtagtacctacactcatgcaaagttcgattggttcgacgaaaggacctacgagtccccgtttttgaaagcctcaagcgaaggtatgaagccgatagctagcgtctggctagtcgcaccacctattttcgaactggctccgcctatgataaagtttgtcgagtttgctagggcgagggaagtaaaacgttttgtcctactaagtgcgagcactatagaaaaaggcgggccagctatgggtcaagtgcatgcccacctggacacaatcaaagggatcgtatatgcagtgctacgtccgacatggtttatggacaatttttctactgctggtgagctacaacaattagccatcaaaaatgatagtaaaatctatagcgcagcgggtgatggcaaaataccatttataagcgctaaggatattgcgaaggttgccgtaaaatgcttaacgaacgggaatttgcaagcgaaagaatatgtgttgctaggtccaaagcttcatacatacgacgaagtagcatctattataagcggtgttgtgggacgtgagattgtccacgttaaactttccgaagcggacttcactaagcaccttataagcagaggtgtacccctatctgacgcagagttcatggcaatcatggaaacagggatcaagaacggcgggcaggaatccctaaacgatgttgtcgaggaagtcacaggctccccaccagagactttcacggatttcgccttccgtcataaggagtgctggatg

SEQ ID NO: 44 - DNA, 876 bp, synthetic construct
  source 1..876: mol_type = other DNA
atgtcatccacgattctgttgacaggcgggagggggaagacagccagtcatattgccgcactgttggatgccgcgaaccttccttttgctattgcatcccgtagttgtagccatgattcaaaataccgtcagcttaggtttgattggcttgatgacactacttatgataacgttctgtcaccaaaggatggaatgcaacccattagtgcagtatggcttgttcctccacctatagcggatcttgcaccgcctgtaaatgcttttataaatcacgcgaggctgaaagatgtcaagaggttcgttctgctatcagcgtccatgctggagagagggtcacctgcaatgggccagattcacgagcacttggcgtcaatggagggcatccattacacggtactgaggccaacgtggttcatggaaaattttagttcacctcatgaatttcaatgcaaggcaattcgtaaggaggataaaatatattctgctgcggagaatggaaaagtgcccttcatctcagtagtcgatatcgcaagggtggcatttcgtgccctaaaaggcgacgtggaggaaaacatagaccctattttgttagggccagaactgctaacttatgacgatgtagccgagacgctaagtaagcaacttaataggaaaattgaccacgtaagactagaggaatcagaattggcagaaaagatgcacgaatacggcatgccgttggaggatgcacagatgcatgctagtatggacctaatcgtaagggccggaggagaggagaggctgaataagactgtcttggagttgacggggaccacacccagaacgtttcaagagttcgcgtctagtgagaaagacatatggcttctgaaaaattaa

SEQ ID NO: 45 - DNA, 876 bp, synthetic construct
  source 1..876: mol_type = other DNA
atgtcatccactatacttgttctaggaggccgtggaaaaacagcctcacgtctttcactactacttgataatgcgggtgtgccattcctagttggaagcagtagcacctcatacgtcggaccatataagatgactcatttcgattggctgaacgaagatacttggacgaacgtatttctacgtgcgtctttggatggaatagatcctatcagcgcagtttacctagttgggggccacgcccctgaacttgtagacccaggtatcaggttcattaacgttgctagggctcaaggcgtcaaccgttttgtgctactgtccgcgtccaacatagctaaagggacgcattcaatgggaatattgcatgcccatctagactcattagaagatgtccagtatgttgttttgagacccacttggttcatggagaatctgctggaggaccctcatgtcagctggattaaaaaggaagataaaatctactctgccactggagacgggaaaatacccttcatctctgcggacgacatagccagagtagccttttctgttctgacagagtggaagagtcaacgtgctcaagagtacttcgtacttggaccggagttactatcttatgaccaagtagccgatatattaacgactgtcttgggcagaaagatcactcatgttagtttggcggaggcggacttggcgcgtcttctacgtgacgatgtgggccttcccccagatttcgccgccatgttggcaagtatggaaacggacgtgaagcatgggactgaggtgagaaactcccatgacgtaaaaaaagtcaccggcagtttaccatgcagctttttggatttcgcggagcaagagaaagcgcgttggatgcgtcac

SEQ ID NO: 46 - DNA, 876 bp, synthetic construct
  source 1..876: mol_type = other DNA
atgtcatccacgatattgttaactggtggtaatgggaaaacagcccgtcacatagcaggattattagaggaagcgaatctgccgtttatcatcggtagtcgtagcaactcccctcaaactattgagagacacaggtcattcgactggttggatgagacgacctttcataacatcttgagcccaggcgagggcatgagaccaatttcagcggtatggttagttccgtccccaatcccggatcttgcatcccctgtaaacaaatttatcgacttcgcctgttcaaaagacgttaagagattcgtcctattgtcagcatccatcgtggagaagggaggtccagcgatggggcaaattcacgcgcacctggatagcctggagggaatcagctatacagtcctaagaccaacttggtttatggaaaatttttccactatggacgattttcaatatgaggcgatcaggaaggaaggaaaagtctattcagcgacgggagatggtaaggtgcccttcataagcgtcgtggacatcgcaagagtagctctacgtgcccttaccggcgacgtgcccgacaagacggaccatatcttattaggtcccgaattgcttacctatgacgacgtagctgagacgctgactaggacactggatagaaaaatcacgcatgccagattgactgagtcagagctggccgaaaagatgcagaagagcggtatgccggcggaggacgctcagatgcacgcttcaatggattctatcatacagtccggagcagaggatagattaaacactgctgtccgtgacttgacaggtttagagcctcgtaacttctccgattttgcactaagcgaaaagaaattctggaggtctagggattaa

SEQ ID NO: 47 - DNA, 864 bp, synthetic construct
  source 1..864: mol_type = other DNA
atggcagccgcaattctattgactggaggaaccgggaaaactagcatacgtctggcgcgtttcctacagaatgcaaacattccgtttttacttgcgtcaagacgtggctcatcagcggcgccggttggtatgcctgcagtcaaattcgactggttggacaaggaaacttggaaggcgcccttcggagggaaacccatatccgcaatatatttaatggaaccactggtggcagagccctggaaacccatgatagagttcattgaatacaccagaaatgtccacggagtaactcgttttgtgctgttggctggaacaagcactgacctgagtcgtccgggtatgggcgtggtttggaaacactttttagagacgggtgtggatcactgcgtcctaaggcccagctggttcatggagaatcttacagatgaagcgccgggtcctttaatcagagagtattccaagatatacaccgcttgtggagacggaaagataccatttgtttcagctatagatattgcggcggtagcgttcagggcgctgaccgatccaaagagccataattgcgattataggatactagggccagaactactgacgtacgacgaagtcgctgagaaattaagtgctcatctggggagaaggatcgagcatgtcaaactttctggagacgagagatataaggggcttaccgatgcgggggtctctgattactttgccaggtttttaacgaacttagaagttgctgctgccgatggttttgaaacccacatgaataacacggtagaagaagtcagagctggtcaccctggagcgtggacgactctactgatgaagatatacatccgtgggtctaggtataactaa

SEQ ID NO: 48 - DNA, 1482 bp, synthetic construct
  source 1..1482: mol_type = other DNA
atgtcatccggctctacgcttatagcaattagcgtgacactattcctaccactaataattagtgccttttacaggttgtataaacatccccttagaaagatacctggtccaagactggcagcagttacaaatctatatgccttttactacaactatattcaggaaggaggttatagcaaacagttcaaagggctacacgagaagtataactccccggtagttaggataggacccaatcatgtccatgttgataagttgagcttttatgacgaggtgtttcgtcccggcacgaagtaccgtaaagacccatcattttacaactcttttggcggtcttgatggcatgatagatgaagacggattccgtacctataggtcccacatctccagcctttactccgctaagagcgttgacatgttagcaccgaaactactgagggaggtacaaactgtcgccgacaggttgcagaaggacgtagggacgaatcaggtcgtgaatattcaaaagatgttcaggaccctatctgcggacatggttttacacattttatttccccaagacattgatttgttcgaatgtgaccaatatcatccctttcttgaaagtttcgacataatgttgacaaaaacttggcttagcctatcttacccgtttgccctgggactgttggcccttttaccatggccagaatacaagcgtgtcggggttgcagccgagaactttgccaagtactgtcaaggatggatgacgaaagcgcaacacctacaaaactctggggcagagaaaggtagggattgtcatataaagagatatttggagctggaccccacaaatgaaaagaaaaaaaaagcaatcccatacccactggaagatattttcaattttatcgccggaggcactgatacgactgcatatattatttcttgcgtggtttattacttattgaaaagtccgaacgttttgagcaacttgcagaaagaattagacgaggctgcgctatgtataagacataaattcgatcacaagcaaatccaaagcctaccctatttaacagccgtaataaaggaagcactgcgtatatctacgcccgctccgggttgcttaccccgtatcgtaccagatggaggagcgcacgtggactccgtatacataccttccggtactgccgtcagcgtctcaatgttaagcattcaacaaaatgctgatctgtttcccgagcctaggaagttcaaacccgaaagatggctatgcgaagagggcaaggcccttgcaaaatggaatatcgcgttttctaggggcccgcgtcaatgcatcgggacgaacgtagcttatttggaattatatggcacgattgcttacctattttcaaggttcgaatttgcacttagtggcaatgacggtgatgaactagagtggatagacaggtttacagcccataacatcgaagatatcaaagtgaaaattaccaaagacaaatggaca

SEQ ID NO: 49 - DNA, 1560 bp, synthetic construct
  source 1..1560: mol_type = other DNA
atgtcatccgctactaatccgtccacacttctaagtgttaccaagcaggagcagggtctgagcttacgttggccgacccagtacggttcttggacgtggatattattaactctatgtatctccttaactacccctttcattgtaaactgtgtctataacttgtatcttcatccgttgaggaatgttcctggacctaagttagccgccgtcacgtccttctatgcattctattggaactggataagggacggtgagtacagcaaaaaattcaatttttggcacaaacattacaactcaccggtaatacgtataggtccgaaccacgtacacaccactgaagcagacatttattatgtcatattcaagccagggtctaagtggcttaaagataaggctttctacaaacattttaatggggttgacgcaatgatcgagccaacgctgtatagaacgtacaggatgcatctggctccactatacagtcaaaggaccatcgatgatctagctcccaaggtctatcaaaacttgtctattttcgggaacagaatgactaaaatggccgaaacaggtaaagcagtaaatatggtgaagattatgaggaccctaagtacatcaatgattttacacattgtgtactctcttgacatacctctatgcgagcacgatggttaccatcctcttttggaggcgtttgagagagtcatgactcagtcatggctttttgttacatatcctattattttcggattgctatccctaatacccggaaccacgattgcgcgtctgaattcttctttcgaggtatttatgaagtattgcaaggagtggaacgacgaggatatgcgtaagcagaggacctcagagggccagtccgccagagacagccatatgaaaagatatcttgagatacaagatgatgaggagaagaatcacatcattccgaacccaattgacgacgtcttcaacttcatcgcaggtgggtctgatacaacgacgtacacaaccgcatgtgcattcttctacatcctgagttctccgatggtccacaagaaaattctagacgagcttgacgccaatagcggcgccataagggatgcctacgactataataagatcaagaacctaccgtacttaaacgccgtaataaaagaaactcttagaatttccgtaccgcttcctggctccatgccaagagtcgtcccggagggtggagttgtcttagggtctttcagcataccagcaggaactgcagtctcaatatcccaccaagccatatcttttaatgaagaaatcttcccgtctagtggggaattcattcccgaaaggtggatagaggcagccgggttagataagtggaacattgctttttcaagggggccaagacaatgccccggtaaaaccctggccttcctggagttacactgtgttcttgcgtactttttctcccgtttcgagatgacccttactggcaattgcgggaaaagtttacgttgggtcgaccgttacgttgccgcaaacttagatgacgtggaagtacgtatcctacgtgataggtggaac

SEQ ID NO: 50 - DNA, 1527 bp, synthetic construct
  source 1..1527: mol_type = other DNA
atggcagccagcttacagtggcttcagcaaacgagacatgaattatcttggacgtggattctgctaaccacctgtattgcgttgactagtcctttggtactaaaaggaatctataatgtatactttcatccactgcgtaatatcccaggacccaaacttgccgctctaacggatttctatgcgttttattggaactggatacgtgacgagggttatagcaaacaattctcaaggctgcacgagcaatacaacagcccgataatcaggatcggaccgaacaacgtgcacaccacgcaagttgaattctatgatgtcatattcaaatcaggatctaaatggttgaaggacaaaagtttttacaaatactttaacggcttagacgctatgatagagccttatcagtatagaacctacaggacacatctggccccactttacgcccaaagagctatcgatggcctagcccctaaacttaggtctgacctaactaactcagcgtcaggaatgatgagacagactgaaaacggacaaacagtgaacatggctaaagttcttcgtacgctttctacgagtatgatcctacataacctgttcagtctggacataagcttaaatgatggggatgagtatcatccgttcctggaggcgtttgagcaactaatgacccaaagttggttgttcgtaacctaccctatggtaccaatggttttaagtttgataccaggaaccagttttgcgaggttcaacagttcctacacaactttcagcaattattgtacggcttggaacgatgaggatatgaggaagcaaagggagagtgagggacagtctacaagggatagtcacacccgtagatacttgtccctaaaagacgatgatgctaggaagaaaacagcgattccctacccactagatgacgttttcaacttcgtagcgggcggatcagatacaacggcatatactaccgcttgtgccttttatcacattctatccagtcccactgtccgtgagaatcttgttgtcgaactggacgagcattccagcatcattagggatgagttcgattataacaagatccagaacctaccttatctaaacgcagtaatcaaagagactttgagaatcagtgtacccgtacccggctcccttccaagaatcgtgccgcagggaggaatcacaatcggcagcttcagtttgcccgccggtacgggcgtctctattacccagcaagcaatctcttttaatgaaaagatttttcctttgcctcacagtttcctgccggaacgttggatcggtcccaagagcgtaggcttggacaagtggaatattgccttctcacgtggtcccaggcaatgcatcggcaccacactagcataccttgaactgcgttgcgtaatagcatattttttctcacgtttcgatatggcgttgactggcaactgtggggaccaactgagatgggtggataggtttgtcgcagtgaacctggatgacgttgaggtgcagatcctggctgatcgttggacg

SEQ ID NO: 51 - DNA, 1524 bp, synthetic construct
  source 1..1524: mol_type = other DNA
atggcagccattttgccgtggttgtctcaattgcaaagcgtcagtctaggcactattttccttacactgtttctagtgattctgacacctcttgttttcacttctgtctacaggttgtactttcatccgcttagaaagataccgggtccgaggactggagctctgactagtttttacggtttctattggaactggattagggacgaaggatactctaaattatttaatcctttgcataagcaatataactcccacataatacgtattggtccgaatcacgttcatattaaccaacctcaagcattcgacgagatctttaaagttgggaccacctggaggaaagactcttccttttataagtatttcaatggtctagacgcaatgatcgagcccacacagtataggacctaccgtacccatctagccccgctatacgcacagcgttccattgacggtctgacacccaagctacacgatgatttagtggttacggctgaaaggatggccaaatccatcgagaatggagagcccgtgaatatggtgaaaatccttagaacattatccacaagtatgatgttatatacgctttattctcaagatatccctttatcccagtacgatgggtaccacccattcttggaagctttcgaattacttatgacgcagagctggctgatgataaattatccgatgatgggaatgatactaggtttaattcccgggacaagtttcgccaagttcaatgctgctttcggtacattcctaaagtattgtaaggaatggaatgacgaagatgagagaattcagaagcttgagacagctgagtcattaagagactcacatatgaaaagatatctagctatagaccccaataacgagataaaaaagaaggtagtaccgcatccgttggaagatatcttcaattttattgcaggaggctcagatacgacgagttacacggcagcatgtgcattcttccatgtactgagtagtagcgaggtacactcaaaacttgtagccgagctggaccaggctagctcagtcataagagatacgttcgactacaataagatccagaaccttccatatctaaatgccgtaattaaagagaccctgagaatttcttgtccggtgccaggctgtcttccaagggtagtgccagagggggggatgaatctggggagcgtaaacttgccagccggtactgtggtgagtattagtcagcttgcaattcacttcaatgaaactatcttcagctcccccgacaaattcatcccagaaaggtggttaggcgacgacaggaaatcaatagaaaagtggaatatagcttttagtagaggccccagacagtgcatcggtaccacgcttgcgtatatggaattgagatgtgtccttgcttattttttctcaaggttcgagttcaaattgacagggagctgcggcgacaaactaaggtgggtagataggttcgtctcagtcaacttagatgatgtggaggtaacgatagtgaaagatagatgggcc

SEQ ID NO: 52 - DNA, 1524 bp, synthetic construct
  source 1..1524: mol_type = other DNA
atggcagcctccttacagtggttatatcaagctcaatgtccctcatggacttggataatgtttactgcgtgcattagtataacgttccccttagtgatgattgggatatacaatatctatttccatcctttgcgtaatgttccgggaccgaaactggctgcactgacaaatttttatgccttttactggaattggataagagatgagggctatagccgtcaattccagagtcttcacacttactacaacagctcagtccttcgtattggacctaatcatgtccacactacggagatcgagttgtacaatgtaatatttaaaccggggtctaagtggctgaaatataagtcattctatcgttactttaatggattagatgcgatgattgaaccgagtcagtatagaacgtaccgtgcacacttggcgccactgtacgcgcaacgtgctattgacgatctttctccgaaaatacacgatgatctaagtagctcagcagctcgtatgattaaaattacagaaacaggcgagacgataaacatggtgaaggtcctgaggaccctgagtacttctatgatcctttataatttatacagcatcgacatttccctgaacgagtgcgacggatatcacccatttttagaggccttcgagcaacttatggctcaatcctggctgtttgtaacttatcctatggtgcccatgctattaggtctaatcccaggcacctcatttgcgaggcttaactcctgctacacgaaatttatgaactactgcatcgcctggaaccatgaagacgtcaggaaacaacaagccagcgaagagcaatcaacgagagattcccacacgaagaggtatttagccataaaaaatgatgatggcttaaaaaagtccattgtccccaatcctctagatgatgtgttcaacttcattgctggcgggtccgacactactagttacacagccgcctgtgcgttttttcatatcctatcaagcccgatcatatgtgcaaaactagtcaccgagctggacgagcattcctctattatacgtgacgcctttgattacaataaaatacagaccctaccgtatcttaatgctgtgataaaagaaaccctaaggattagtgtaccagtccccggctgtttgccaagggtagttcccgagggcggtataacgctaggctctcttagtctgcccgctggcaccgctgtttcaatttcacaacaagcaattagtctgaataatgaaatcttctcttctccccatcgttttagcccggagcgttggattgggccagaggcagtgggcctggataaatggaacatagcctttagtcgtgggccaagacagtgcattggcacaacactagcgtatttggaattgaggtgcattctagcttacttctttagcaggttcgagatgactttaaccggcaattgtggggaccaccttagatgggtagatcgttttgtggccgtgaacttagacgatgtcgaggtgctgatacttagagacagatggagc

SEQ ID NO: 53 - DNA, 1542 bp, synthetic construct
  source 1..1542: mol_type = other DNA
atggcagccgaaaataactttacgacggccagtacggtctcaataagtgagtccgggctaatatggcaaaggcttagtcagctacagcaagcaagctgggctttgataatcttcactacatgcgtggcggtcttactacctgccgtgatcaccggcgtttatcgtttatatttccaccccttgcgtaacgtcccaggcccaaaattggcggcgcttacaaacttctacgcattttattggaattggataagagacgagggctattcacgtagattttctgccttacacaagtactataactcacctattatccgtatcggacccaattcagtgcatactaaccaaccggatgactttgatgttatattcaaagtaggaagtaagtggaaaaaggacaacagcttctacaaatactttaacggattggacgctatgatcgatcctgctcaatacagaacttacaggagtcacctggcacctttatacgctcaaagggctgtcaatgacctagcccccaaactacataaggaccttctatcctcagctgccaagatggaaaagtcaatgaaaacatctgaagctgtaaacatggtaaaagtacttcgtacccttagtacgtctatgatactacataacctatactctcatgatattagcttgatagaatatgacggttaccatccattcctagaagcttttgagcagctaatgacgcaaagttggttgtggttcatccctggcacatctttcgccaagttcaattcttcctacactacctttatgaagtactgtaaaggttggaacgacgaggatatgcgtattcagaggctaactgagaaccaagaactgagagactcacatatgaagagatacctagcaatgaattccaatgactgggcgaaggacaatgtcgttcctaaccctctaaacgatatctttaacttcgttgcgggaggttcagacaccactagttatactctggcctgtgcttttttctacatcttgtccagtcccgaaatatatgctaagttggttaaggagcttgatgaaaatgtggcatttattagagatacgtttgattataataaaattcaaaatctgtcctacctaaatgcagtcataaaggaaactttaaggatttctgtccctgtgcccggttgccttcccagaattgtgccagaatcaggtacgacacttgggagcgtctatctgcccgctggcacggctgtgtctatttcacaacagagcatctcattccatgagacaatctttccttcccctgaaaaattcatacccgagaggtggcttggtaatgaagcccagcgtttagacaaatggaacgttgctttttctagggggcctagacagtgtatcggcactagtctagcctatctagaacttcgttgcgtccttgcatacttcctttctcgttttgaaatggcgttgactgggaactgcggggataggcttagatgggtggatcgttttgttgcggtcaacttggatgatgtggaggtaaagctgcttagagacagatggaat

SEQ ID NO: 54 - DNA, 1497 bp, synthetic construct
  source 1..1497: mol_type = other DNA
atggcagccggacttgaggccctagttccattagccgttatagtcgccatcttcttgctgcttgtggatctgatgcacaggaggcagaggtgggcagctagatatccgcccggtcccttgccgttacctggattaggcaatttattacacgttgactttcagaacaccccgtactgttttgatcaacttcgtagacgttttggggacgtatttagtttgcagctggcatggacgcccgtagtagtcttgaatggattagctgcagtcagggaagcgctggtcacgcacggcgaggatacagcagataggcccccggtacccataacgcaaattctgggtttcggaccaagaagtcaaggcgtgtttctagcaaggtatggccccgcttggcgtgaacagagaagattcagtgtctcaacgcttcgtaatttgggattgggcaaaaaaagccttgaacaatgggtaacagaggaagctgcctgcttgtgtgcggcctttgcaaaccatagtggacgtcctttcagaccaaacggattgctagataaagcagtctccaacgtgattgccagcttgacatgtggcagacgttttgagtatgatgacccgcgttttctacgtctactagatctagcccaagaaggcttaaaagaggagtctggttttttaagggaggtcttgaatgcggtacctgtattgttgcacatccccgccctggcgggcaaagtcctaagatttcaaaaggcctttcttactcagttagatgaactattaacagagcataggatgacgtgggaccccgcgcagccgccccgtgacctgaccgaagcgttcttagcagaaatggagaaggcgaaaggtaacccggagtcttcatttaacgacgaaaatctaaggattgtagtcgcagaccttttctccgccgggatggtcacgactagcaccactctggcttgggggcttcttctgatgatcctacaccccgatgtccagagaagagttcagcaagagatcgacgatgtgatagggcaggtaagaagaccagaaatgggtgatcaagcccacatgccttacactactgctgtgattcatgaagtgcagagatttggcgatattgttccgttgggtgtaactcatatgacatccagagatattgaagtgcaaggattcaggattcccaaggggacgaccctaattaccaacttgagcagtgtgctgaaagatgaggccgtttgggaaaagccgttcaggttccatccagaacatttcctagatgcacaaggacattttgtaaaaccggaggcgtttcttcccttttcagcaggacgtcgtgcctgtcttggtgagccattagccaggatggaattatttctattcttcaccagtttactacaacattttagcttcagcgtgccgaccgggcagcccaggccaagtcaccacggagtattcgcgttccttgtatctcctagtccatatgagctttgcgctgtccccagg

SEQ ID NO: 55 - DNA, 1506 bp, synthetic construct
  source 1..1506: mol_type = other DNA
atggcagccggtcttctgataggtgatgtactgggtccgctggcaattgccgttgcactgttcttgctgctagtcgacctgttgcatagaagaaggttttgggcagcgagattcccccccgggccaatgcccctaccattgctggggaaccttttacaaatggactttcaagatacgcttttttatttcgacaggctaagaagaaggtttggggacgtcttcagcctgcaattagcttggacgccagttgtagtactgaatggcttggaggccgttagagaggccctagtctatcatagcgaggacacagcagatagaccgcccatgccgatatacgagcatctggggtttggtccacgttcacaaggggtattcatggcaaggtatggaagggagtggagagaacagcgtcgtttcgcactgagtactttaaggaactttggcctgggcaaaaaaagcctagagcagtgggtaaccgaagaggcatcctgtctatgtgcggcctttgctgatcatgccggccgtccctttagtccaaaagcacttcttaataaagctgtgagtaacgtgatcacttcattgacgtacggtcgtcgttttgggtacgacgacccccgtctgcataagttgattgatggcgcattgaagggcttgcaagaagattctgggtttgctcgtgaagcactgaattcaataccggttttactaagaatccccggattagccgataaagtatttagtagccagaaggctttgcttactcttctgaacgaacttgtacaagaacataggatcaccagggacccagcccagccgcctcaggatctgacggacgccttcttagatgaaatcgagaaagcgcgtggaaatcccgagtcttctttcaacgatgaaaacatgctgatggtaacagctgatctttttctggcggggatgctaagcacatctacgacacttgcctgggccttgctgttgatgatcttacaccctgacgtccagcgtcgtgtccagcaagagatcgatgaggtgctaggacctgtccaacgtccggcgatggcggatcaaacacgtatgccatttacaatggccgtcatgcacgaagtgcaaagatttggggacttggttcctcttggcatgccccatatgaccagtagggacatcgaggtacagggatttttgataccaaaagggacaacacttatcaccaaccttagttctgttcttaaggacaagacagtttggaaaaagccgttccgttttcaccccgaacacttcttggacgctcagggtcaatttgttaagcaggaggccttcatgcccttcagcgcggggagaagagtgtgcttaggagaaccactagctcgtatggagttttttctatttttcacgtgcctattgcaaagattctccttttctgtgcctgtagggcagcctaggccctctgatcacggagtttttagctttctgatgatccctcccccttatcagctgtgtgctgtgccgagg

SEQ ID NO: 56 - DNA, 1485 bp, synthetic construct
  source 1..1485: mol_type = other DNA
atggcagcctccgcgctgggagttactgtcgcactacttgtatgggcagcctttttgctgttggtatcaatgtggcgtcaagttcacagttcctggaaccttccccctggacctttcccattgcctatcatagggaacttgtttcaattggagcttaaaaacatccccaaatcctttactcgtctagcacaacgttttgggccggtgttcacgttatatgttgggagtcagcgtatggtagtgatgcacggctataaggcggtgaaggaagctcttttggactataaggacgagttctccgggagaggtgatttgcctgcattccacgctcacagggaccgtggaatcatattcaataatggaccgacttggaaggatattagacgttttagtttgacaacacttaggaactacggaatgggaaaacaggggaatgagtctaggatccaaagagaagctcattttctgttggaagcgctgagaaaaactcagggacagccatttgatcccacgttcttaatcggctgtgcgccatgcaacgtgatcgccgacattctattccgtaaacactttgattacaatgatgaaaaatttctaaggctgatgtatttgtttaatgagaatttccacctgctgtccactccgtggctacagttatacaacaatttcccttcatttttgcactatttacctggcagtcatcgtaaagtcataaaaaatgtcgcagaggtaaaggaatacgtatccgagcgtgtcaaggagcaccaccaaagccttgacccaaattgtcccagggatttaacagattgcttgctggtcgaaatggaaaaagaaaagcactccgcagaaaggctatacacgatggacggaatcacagtgactgttgcagatttattttttgctggcacagagaccacgtctaccactttaagatacggtctgctaatcctgatgaagtaccctgaaatagaagaaaagttgcatgaagaaatcgacagagtcatagggccaagtagaatcccggctatcaaggacagacaagagatgccttatatggatgccgtggttcatgaaatacagaggttcataactttagtcccttccaatctaccccatgaagctaccagggatacgattttcagaggttaccttataccaaagggcaccgtcgtggtacctactttggattccgtgttatacgacaaccaggaattccctgatccagagaagtttaaacctgagcactttctgaacgaaaatggcaagtttaagtactctgactatttcaaaccgtttagtacagggaaaagggtatgtgcaggcgaagggctggcaaggatggagctattcttactactatgtgcgattcttcagcatttcaacttgaaacctcttgtggatccgaaggatatcgatctgtctcctattcatattggatttggctgtatcccaccaagatacaagctatgcgtaatcccaaggagt

SEQ ID NO: 57 - DNA, 1485 bp, synthetic construct
  source 1..1485: mol_type = other DNA
atggcagccgcagtattaggaatcacagtcgcattgttggtttggattgcgactttactactagtgagtatctggaaacaaatctatagaagctggaatctgcctccaggaccatttccaattcccttctttggtaacatatttcagttggaccttaaggacataccgaagtcattgaccaaactagcgaaaaggttcggtccagtgtttaccctgcacttaggacagaggaggatagtcgtcttgcatgggtataaagccgtcaaagaagtattgctgaaccataagaacgaattctcagggaggggtgacataccagttttccaagagtataaaaacaagggtatcatattcaacaatggacccacgtggaaggacgtgaggagattctctttgtccatactaagagactggggaatgggtaagcaaggcaacgaagcaagaatccaaagagaggcacacttcctagtagaggagctgaaaaaaaccaaggggcaaccgtttgatccgactttcttaatcgggtgcgctccttgcaatgttatcgccgatattttgttcaacaaaagattcgattacgacgataagaaatgccttgaactaatgagtctgttcaatgagaacttttatttgttatcaaccccctggatccaggcatacaactatttctcagattatttacagtacctgcctggctcccatcgtaaagttatgaaaaacgtatctgaaattcgtcaatacactttgggcaaagccaaagaacacttgaaatcccttgacatcaactgcccacgtgacgtaacagactgtttactgatagagatggagaaggagaagcactcacaagagccaatgtacaccatggaaaacatcagtgttactctggccgatttattttttgcgggcacagagaccacttctacaacgctgagatacgggcttcttatattaatgaaatatccggaaatcgaggagaagctgcatgaggaaatagacagggttattggcccgagcagggccccggctgtaagggatagaatgaatatgccttatatggatgctgtcgtacacgagatccagaggttcatcaatttagtaccaagtaatttgccgcatgaagccacacgtgacacagtcttccgtggatatgtcattcctaaaggaacggtagttattccaaccttggattcattgttgttcgataactacgaatttccagaccctgagacctttaaacctgaacactttcttaatgaaaacggaaagttcaagtatagtgattacttcaaagccttttcagccggaaaaagggtgtgcgtgggagaaggcttagcacgtatggagctttttctactgctttctgcaatattgcaacatttcaacttaaagagtttggtagaccccaaagacatagacctatcccccgttactataggatttgggtccatccctcgtgagttcaaattgtgcgttatacccagaagc

SEQ ID NO: 58 - DNA, 1515 bp, synthetic construct
  source 1..1515: mol_type = other DNA
atggcagccgcacttatcccagatctagcaatggaaacgtggttattactggcggtgagcctggtccttctatacctttatgggacgcacagccatggcctatttaagaagttggggatccccggtcctaccccccttcccttcttaggtaacatcttgagctaccataagggattctgcatgtttgatatggagtgtcacaaaaaatacgggaaggtttggggattctacgatgggcagcagcccgtacttgccattactgaccccgatatgattaaaacggtcttagtaaaagaatgttactcagtctttacaaatcgtagacccttcggacccgtcgggtttatgaagagtgctatatctattgccgaagacgaggagtggaagcgtttaaggtctttactgagtcctacttttacctcagggaagttgaaggaaatggtcccgattatcgctcaatatggagacgtattggtacgtaatcttaggagggaagcggaaactgggaaacccgtaacactaaaagatgttttcggcgcatactcaatggatgtcattactagtacgtcttttggagtcaacattgactctttaaacaacccgcaggatcccttcgttgagaacactaaaaagcttttaaggtttgacttcttagatccattcttcttaagtattacagttttcccattcttgatcccaattttagaagttctaaacatttgtgttttcccgagggaagtgacgaactttttgcgtaagtcagtgaaaagaatgaaggaaagcagactggaagacacccagaaacatcgtgtggattttttacagcttatgatagattcccaaaattctaaggagactgagagccataaagctctaagcgacttggaattagttgcgcagagcatcatcttcatatttgccggctacgaaacgacctcctctgtacttagctttatcatgtacgagctggctacgcacccagacgttcaacaaaagctacaagaggagatagatgcagtccttccaaataaagctcctccaacgtacgatacagttctacaaatggaataccttgacatggtggtgaatgaaaccctgagattgtttccgattgccatgagattggagcgtgtgtgcaaaaaggatgtagagatcaatggcatgtttatcccaaagggagtagtagtgatgatcccctcatatgcgctacatagagaccctaaatattggacagagcccgaaaaattcttgcccgagaggtttagcaaaaagaacaaagataatatcgatccgtacatttacacacccttcggctccgggccgaggaattgtataggtatgagatttgctctgatgaatatgaagttagccctgattagagtgttacaaaatttctccttcaagccatgcaaggaaactcagataccgcttaagttaagtctgggtgggcttttacagcccgaaaagccagtggtgcttaaagtggagtctcgtgacgggacagtcagcggcgca

SEQ ID NO: 59 - DNA, 2037 bp, synthetic construct
  source 1..2037: mol_type = other DNA
atggcagccggcgacagccatgtcgatacttcaagcactgtgtccgaagcagtggcagaagaagtttcattgttttccatgacggacatgatccttttctccttgatcgttggtttgttaacgtattggttcttattcaggaagaaaaaggaagaggtgcctgaatttaccaaaatacagactttaaccagttcagtacgtgagagtagttttgttgagaagatgaaaaagaccgggagaaacattatcgtgttttacggcagtcaaaccggtaccgccgaggaattcgcaaacagactttctaaggatgcccacagatatggtatgagaggaatgagtgcagacccagaggagtatgacctggcagatctgtcctccctaccagagattgacaatgcactggttgtgttctgtatggctacctacggtgaaggagatccgacagacaatgcgcaggatttctacgactggttgcaagaaaccgatgtagatttatctggcgtaaaattcgctgtctttggtctgggaaataaaacctatgagcattttaatgcaatgggtaagtatgtcgacaagcgtctggaacagttgggggctcaacgtattttcgaattaggcttaggggacgatgacggtaacttggaggaggactttattacttggcgtgagcaattttggcctgcagtgtgtgaacattttggagtcgaggcaactggggaggagtcaagcatacgtcaatatgagttagtggtacacaccgacatagacgcagcgaaagtttatatgggtgaaatgggtagattgaagtcttatgagaatcaaaagcccccgttcgatgcgaagaatccattccttgccgccgtcacgaccaataggaagctgaaccagggtaccgaaaggcatttgatgcatcttgagttagatattagcgattccaaaattagatacgagtcaggcgaccacgtagcagtttatccggcgaacgacagcgccttagtaaaccaacttggtaagattttgggagctgatctggacgtagtgatgtccttgaacaacttggacgaggagagtaataaaaagcacccgttcccttgtccgacgagttacaggacagcgttgacatattatctagacataacaaaccctccgaggacgaacgttctttatgagctggcgcagtacgcatccgaaccctcagagcaggaacttctgaggaagatggccagttcctctggtgaggggaaggaactgtatctaagttgggtcgtagaagccaggaggcatattcttgcgatcttacaggactgcccctctttgagaccacctatcgaccacctttgtgagctgttgcccagattacaggcgagatactactctatcgcctcatccagcaaggtccacccgaattctgtgcacatttgtgccgttgtcgttgaatacgaaaccaaggctggtaggataaacaagggagtggcgacgaattggcttcgtgctaaggaaccggcaggcgagaatgggggtcgtgcgcttgtgcccatgtttgtacgtaagtcccagttccgtttaccgttcaaagcgacgactccggtgattatggttggaccaggtacgggggtggcacctttcatcggtttcattcaggagagggcatggttaaggcaacaaggcaaagaagtaggagagactctgttgtattatggctgcagaagatctgacgaggattacttataccgtgaggagttagcacagtttcaccgtgacggtgctttaacgcagttaaatgtagcattcagtcgtgaacagagtcataaagtctacgtacaacatctgttgaagcaagaccgtgaacacctatggaaactaatagaggggggagcacatatttacgtctgtggggatgcgagaaacatggcaagagatgtccagaacactttttatgatatagtagctgagttaggtgctatggaacacgcacaggcggtcgattacattaaaaaactaatgacgaaaggcagatattcattggacgtgtggagt

SEQ ID NO: 60 - DNA, 2091 bp, synthetic construct
  source 1..2091: mol_type = other DNA
atggcagccgcacaactagacacccttgacatcgtggttcttgttgtgttactagtggggtctgtcgcctatttcaccaaaggctcctactgggccgtgcccaaggacccgtatgcggcggctaatagcgcgatgaatggtgctgctaaaacggggaagaccagagacataatccagaagatggaagaaactggtaaaaactgtgtgattttctatggcagccagacgggcacggcggaagactacgcatctcgtctagccaaagagggttcccaacgttttggacttaaaaccatggttgcagacttagaggactacgattacgaaaatttggacaaatttccagaagacaaaatagcctttttcgtgctggccacgtatggagaaggtgagccgactgacaatgcggtagagttctatcaattcattacaggggaggacgtagcatttgagtcaggagctagcgcagaggaaaaacccttatcttccttgaagtatgtcgcattcggtcttggaaataatacatacgagcactacaacgctatggttaggcatgtcgacgcggcgcttactaaattaggtgcccaaagaattggaaccgccggagagggtgatgatggcgcggggactatggaagaagacttccttgcttggaaggaaccgatgtgggcagctttatctgaaagtatgaatctacaagaaagagaggctgtgtatgagcccgtatttagtgtgatcgaagacgaatctctgtcacctgaggacgatagtgtctatttgggagaaccaacacagggacatttgtccgggtcccctaagggcccgtattcagctcataatccatatatcgcgcctattgttgaatcccgtgagctgttcactgccaaagaccgtaactgtctacatatggagataggcattgccggtagtaaccttacgtaccagactggagatcacatcgcaatttggcctacgaacgcaggggttgaggtcgacaggtttcttgaagtgtttggtatcgagaaaaaacgtcatactgttatcaatataaagggacttgacgttaccgccaaggtaccgattcctacccctacaacatatgacgctgccgtccgtttttatatggagatctgcgcacccgtctccaggcagttcgttagttccctggttcccttcgcgcctgacgaggagagtaaagccgagattgtgaggttgggtaatgacaaggattacttccatgagaaaataagtaaccagtgtttcaacatcgcgcaggcattgcagaacataacttcaaaaccgttctctgccgtgcctttctccttactaatagaggggctaaaccgtctgcagccgcgttattattctataagcagtagctctcttgttcagaaagataaaatatctattactgccgtcgttgagtcagtcaggttaccaggggctagccacatagtcaaaggagtaacgacgaactatcttctagcgttgaagcagaaacaaaacggggacccgtcacctgaccctcacggcttgacctacgctatcacgggaccaaggaacaaatatgatggaattcatgtgcccgtgcatgtacgtcatagtaactttaaacttccatctgacccgagcaaaccgattatcatggtaggtccgggcacgggagtcgcgccctttagggggttcatccaggagcgtgcagcattggctgagtccggcaaggacgtgggaccaactatcttgttctttggttgcagaaaccgtaacgaagatttcttatataaagaagagtggaaagtttaccaggagaagttgggcgataagttaaagattatcaccgcattctctagggagacggcaaaaaaagtgtacgtgcagcataggcttcaagaacacgcggacttggtctctgacctgttgaaacaaaaggctacattttatgtgtgcggtgatgcggccaatatggctcgtgaggtaaatctagtattgggtcaaattatcgcgaaatctagaggcctgcccgctgaaaagggcgaagaaatggtcaaacatatgagatcaagtggctcttatcaagaggacgtatggagc

SEQ ID NO: 61 - DNA, 2091 bp, synthetic construct
  source 1..2091: mol_type = other DNA
atggcagccgcccaactagataccctagatcttgttgtattagcagtcctgtttgtcgggagtgttgcgtatttcaccaaggggacgtattgggcagtcgcgaaagacccttatgcgtcaacaggtccggctttgaacggagcggccaaggctggcaaatcccgtaatatagtggagaaacttgaggagaccggcaaaaactgcgttatattctacggatctcaaaccggaacggcagaggattatgcatctcgtttggctaaggagggttcacaaaggttcggcctgaaaactatggtagctgaccttgaggactatgattacgagaatcttgaccagttcccggacgataaggtagcatttttcgtactggcaacatatggggagggcgagccaactgacaacgcggtcgaattttatcaatttctgaccggagaggacatagccttcgagtctggcgcatccgctgaggacaagcctctgtcatccttaaagtacgtcgcttttggtctagggaacaatacatacgaacactacaacagtatggtccgtaatgtcgatgcggctttccagaaactgggggcacagcgtatagggaccgctggcgaaggcgatgacggcgcagggaccatggaggaagacttcctggcgtggaaagaaccaatgtgggccgctctgagcgaggccataggtctagaggagagggaggccgagtatgaaccggttttttcagtcacggaagatgattccctgtctccagaggatgacagtgtatatctaggggaggccacaaagggccacttggaagggtcaccaaaagggcccttttccgcccataatccctacatcgctccgatcgtcgaatccagagaattgtttaccgtaaaagataggaactgcctgcatatggaaattagtatcgcagggtcaaatctgtcctatcagacgggtgaccacatcgctatatggccaacaaatgcgggcgcagaagtggaccgttttttacaggtatttgggttggaaggtaaaagacacacagtaattaacataaaaggcattgatgtgacagcaaaggtgccaatcccgacgcctacaacttacgatgcggcagttcgttactatctagaagtttgtgctccggtctcccgtcagtttgttagtagcctagcggctttcgcacccgatgaagaaagcaaggcggagatagtcaggttaggaagcgacaaggactattttcacgagaagatatctaatcaatgttttaacatcgcgcaagccttacagtctattacttcaaagcctttttctgcggtccctttttctttgttaatcgaggggctgaccaaactgcaaccgcgttactatagtatctcctctagctctttggcccaaaaggataagatatccgtaacagccgtggttgaatcagtacgtttacctggcgccagccatatcgtcaaaggggtgacgacaaattatctactggctcttaagcaaaagcaaaatggtgatccctccccggaccctcatggtcttacatacgaaataaatggacctaggaacaagtacgacggaatccatgtcccagttcacgtacgtcactctaatttcaagcttccttcagaccccagccgtcccgttataatggtcggtcctggcacgggtgtagcccctttcagggctttcatacaagaaagggcggcgcttgcggcgaggggcgagaaggttgggcccacggtgctgttctttggctgtaggaacagaaacgaagactttatgtatcaagacgagtggaagatttaccaagagcagctaggagacagtcttaaaatcattaccgctttctcaagggagtcatctcaaaaggtttatgttcagcataggctttctgaacacgctgagttggtatctgatttactaaagcaaaaagccaccttctatgtctgtggtgatgctgctaatatggcgagagaggtcaacctagttttgggccagattatcgctaagcaaagaggtcttccggcagaaaagggcgaggagatggtaaaacacatgcgtagtagtggctcatatcaggaagacgtttggtca

SEQ ID NO: 62 - DNA, 2082 bp, synthetic construct
  source 1..2082: mol_type = other DNA
atggcagccgccgagttggataccctagacatcgttgttctgggagcgatcctgttcggaacgctggcctacttcaccaaaggaactctgtggggcgtaacgaaggatccatatgcggatgctttcgccaaggtgtccggcgccaaggctgggcgttccagaaatataattgagaagatggaagagtccggcaaaaattgtgtcattttctacggctctcagacaggtactgcggaggattatgcaagtcgtttggcgaaagagggaaaatctagatttggcttggaaacaatggtcgcggatttggaggagtttgattacgacaacttagatacaatgccctctgacaaagtagccatgttcatattggccacgtacggtgaaggcgaaccgactgataatgcggttgagttctacgagttcatttctggtgaagacgttagtttctcagaaggcaatgatccgcccttggataatcttaattacgtcgcctttggtctggggaacaatacgtacgaacattacaactccatggtccgtaacgtagacaaagctttacagaaattgggagcgcacaggatcggagaagcgggagagggtgacgatggggccggcaccatggaagaagattttttagcctggaaggacccgatgtgggctgcacttgcggaaaaaatggggttagaggaacgtgaagctgtctatgagccgactttcgccatcgtccaccgtgaaaacttgacaccggagtcccccgaggtatacttaggagaacctaacaagatgcacttggatgggattgcaaaaggaccgttcaactctcataacccgtacattgcaccgataacagagagcagagaactattcagtacaaaagaccgtaactgcatacacatggaagtagacatttccggtagtaacctttcataccaaacaggtgaccacattgccgtttggccaaccaatcccggagacgaagtggacagattcttagatatagtggggctgaaagagaaaagaaatcacgtcatctcagtgaaagcgttggagccaacagccaaagtaccatttccgaccccgactacttatgacgccattgttagatatcacctggagatctgtgccccggtttctagacagtttgtggccaccttagcggccttctcacccgacgaagataccaaagcagaaatgacgagactggggagcgataaggactactttcacgacaaaactgcgccccattattataacatagcgcgttttctggaagctgtaggtaaagggcaaaagtggactaacattcctttctcagcgttcattgaaggtttaaccaagctacagccacgttactattctatttcaagcagctccttggttcaaccaaagaaaattgcaattacggccgtgattgaatctcaggtaataccgggccgtaaggatccttttagaggagtcgccacgaactatctttttgcacttaaacaaaagcagaatggagatcccaatccaaccccgtttggacagacctacgcacttaatgggccaagaaacaagtttgacggtattcatgtcccggtacatgttaggcactcaaatttcaaacttccgagcgatcccgctagacctattataatggtggggccgggaacgggtgtggctccttttaggggtttcgtgcaagagagagctaagcaggcgcaagatggtgttgcagttggtaagacgatcttgttcttcgggtgcaggaagaggacggatgattttttatatgagagcgaattcgaagagtataaaaagatacttggggactcattcgagatagttaccgctttttcacgtgagactccagaaaaggtgtatgtccaacaccgtttaaaagggaggtcaaaggaggtgggtgaattactgagccagaaggcgtacttttacgtttgcggcgacgccgcacatatggcgagggaggttaacactgtgttggcacaaattatcgccgaggcaagaggagtacctgaagccaagggtgaagaaatagtgaaaaacatgagagctgcgaatcagtatcaagaagacgtgtggtcc

SEQ ID NO: 63 - DNA, 2082 bp, synthetic construct
  source 1..2082: mol_type = other DNA
atggcagccgcggagttggacacgcttgacatggtcgttcttggcgtgatactattaggtactcttgcatatttttccaagggaacaatatggggtgtcgtgaaagacccatacgcaaacacttttgctaatgtaaatggcgcaaaggcagagaggtcacgtaacattatcgagaaaatggaagaatccggtaaaaattgtgtcatattctacgggtcacagactgggaccgcggaggattacgcatcccgtctggccaaggaaggaaaatctaggtttgggttggagaccatggttgccgatttagaagaatacgactatgacaacctagatacgatgccatccgacaaggtagcaatgtttgtgctggcgacatatggggaaggcgaacctaccgacaatgctgtcgaattttatgaatttataaccggggaggatgtcagcttttctgaggggggcgatgatccactacaaaacctaaactatgtggcatttgggctgggtaacaatacctacgagcactacaactctatggttagaaacgtggacaaagcgctaacgaagctgggggcgcatcgtatcggccaagcaggagagggtgatgatggggcaggaactatggaggaggattttctagcttggaaggacccaatgtgggtagcgttagctgaaaaaatggggctggaagagagggaggcagtttacgaaccgaccttcagtattgtcgacagggagaatttgactccggagagtcccgaagtgtatttgggtgagccaaacaaaatgcatttggacggcacggcgaagggaccgttcaacagtcataacccttacattgcccctatcgtggagagtcgtgagctattcagcgcaaaggatcgtaactgcattcacatggaaatagatgtaagtgggtctaacttaacctaccagacaggcgatcacctagccatctggccgactaatccaggggaagaggtcgacagattcctagacgttattggtttgaaagaaaagcgtaacaatgtgatttcagtgaaaccattagaaccgacagccaaggtgccatttccgaccccgacgacttatgacgctattgtcaggtatcatttagaaatctgcggtccggtctctaggcaattcgcagccacggtggctgctttttcaccggatgaagagatcaaggcggagatgacgagacttgggagtgacaagcagtatttccacgagaagacgggtcctcatttctacaatatcgctcgtttcttagaagcaatcgggaaagggcaaaaatggtcaaatataccgttcagtgcgtttatcgaaggcttaactaaattgcaacccagatattattcaatctcaagttcctcccttgtgcagccaaaaaagatctccctaacggcggtagttgagtcccagaaaatcgtcggtagaatggagcccttcaggggggtagcgaccaattacttatttgctctgaagcagaaacagaatggcgaccctgatccatcatccttcgggaagacatatgcactaaccggtccgcgtaacaagtttgacgggatacatgtcccggtgcacgtgaggcactcaaactttaaactaccctccgatcctggcaagccgatcataatggtaggaccggggacgggagttgcgccgttccgtggtttcattcaggaaagagctaagcaggcccaaaatggcacatctgtaggaaagacgattcttttttttggatgtcgtaaaaggtccgaagactttttatacgaaagtgagttcgaagagtacaaaaagactctaggggataatcttgagatcgtcactgcgtttagtagggagggttccgaaaaggtctacgtgcagcatagattgaaagagaggtctaaggaagtaggggaacttctaagtcagcgtgcctatttttatgtatgcggcgatgcggcgcacatggcgcgtgaggtcaacacggttttagcacaaatattagcagaatcacgtggtgtttccgaggcaaaaggcgaggaaatagtcaaaaacatgcgtgccgctaaccagtaccaagaggacgtctggtca

SEQ ID NO: 64 - DNA, 2094 bp, synthetic construct
  source 1..2094: mol_type = other DNA
atggcagccgcacaacttgatacccttgacattatcgtattggctgcactgctggtgggcacagtggcttacttcactaaggggacctactgggctgtgtatgctgacccgtacggaaatagtcttgggtctgcgaacggagccgcgaaggccggaaaatcaagaaacatacttgagaaaatggaagagactgataagaattgtgttgtcttttatggcagccagaccgggactgcggaagactacgctagtcgtctggccaaggaaggacatgctcgttttggtctaaacacgatggtggccgatcttgaagagtatgactttgataaccttgacagcttccctgaagataaattggctgtttttgtgctagcaacatatggagagggagaaccaactgacaacgccgttgagttttacgaatttataggcagtgaggatgttagtttcgctgatgggggcagtgcagatgataagcctttgtcaaagctgcactacgtcgcttttgggctgggaaataatacttacgagcattacaactctatggtacgtaatgtagataaatatttaacaaaactgggggccagccgtctaggtactgctggtgaaggggatgacggtgcaggcaccatggaggaagattttttggcgtggaaggagcctatgtgggcggccgtatccgagaagatgggattagaagagcgtgaagcgatgtacgaacccactcttgaaatcaatgagcgtcccgagttgagcgtggaagacgacactgtttatttaggagagcctaacaaaaatcacctagaaggtaatcaaaagggtccatttaacgcaaataatccgttcatcgcaccaattgtcgaaagcgcggagctgtttacatctaatgacagaaactgcctgcatatggagataagcattgccgggagtaatctgagctatacaacgggcgatcatattgccatttggcctaataatgcgggaaaagaagtagatagggtatttaaagtgttagggatggaagacagaagacatactgtgattgctgtcaggggacttgacccgacagcgaaagtcccttttcccagtccgacgacgtatgatgccgctgtgaggtatcatatcgagattaatgcggctgttagtaggcagcttgttagtgtaatagcgcagttcgcgcctaacgaggaaataaaagcagagatggtcaagttagggtctgataaggattattttagggaacatgtaactgagagaaacttaaacctggcgcaattacttgagattaatgggaagggtcagacctgggataaaatacccttttcattcctattcgaaactatggtcaaactacaacctaggtattacagtataagtagctcatcattggtacaaaaggataagatagcaataacggccatagtggaatcaatagaaaaaccaggggccccgtacgccttaaagggagtgacgaccaactatctacttgctctgaaacagaaacaacatggggctcccgagccggacccacatggattaaactacgccatcactgggcccaggaacaagtatgacggcattcatgtgccggtgcatgttaggcacagcaatttcaagttgccgtccgacccttccaagcctattataatggttgggccgggcacaggagtggcgccttttaggggattcgtacaagaaagagcggcccaggctcaggcagggcagagcgtaggaaaaacggtcctgttctttgggtgtaggaagagaagtgaagactttatgtatgcagacgaatggaaacaataccaagaggcgttgggcgataactttaagatgcacacagcgttcagccgtgacggtcccaagaaagtgtacgtccagcacaaattagaagaatttggcgaggaggtcaacaaacttctagaacagaaggcttacttctatgtatgcggcgacgcagcgcacatggctagggaagtaaacacgcttcttggtaaaataattagtcagtatagaaacgttagtgaaactaagggggaggagatagttaaggccatgcgtgcaagcaatcagtatcaggaggatgtatggagc

SEQ ID NO: 65 - DNA, 2082 bp, synthetic construct
  source 1..2082: mol_type = other DNA
atggcagccgcggaactggacacgttagacattatcgtgctgggcgtcatcttactgggaactttggcatactttaccaagggcacactatggggggttacaaaagacccatatgcgaatgcgtttgcgaacgtatcaggcgcaaaagccgggaggagtagaaacatcatagagaaaatggaagaatccgggaagaattgcgtcatcttctatggctcacagaccggcactgcagaagattacgcttcacgtcttgcaaaagaagggaaatcaagattcgggctagagacgatggtggcagacttagaggagtatgattatgacaatcttgacactatgccttctgataaggtcgcgatgtttgtacttgccacatatggggaaggagaaccgactgacaacgccgtagaattctacgagtttattacgggtgaagatgtttcattctcagagggatcagatcctccactagaaaatttgaatttcgtagcattcggcctaggtaacaacacgtatgagcattataacagtatggttagaaacgtagacaaggctctacagaaactaggcgctcatcgtataggagaggccggagaaggcgacgacggcgcaggcacgatggaggaagattttttggcgtggaaagacccaatgtgggcagcgttagcggataagatgggattagaggaaagagaagcagtctatgagccgacatttgatatcgtgaacagagaaaacctaacccctgagagtccggaagtatacctaggtgaaccgaataaaatgcacctggaaggggccgccaagggtcccttcaatagtcacaacccttacatcgccccgatcgcggaaagtagagagcttttcagtgcgaaggacagaaactgtattcatatggaagtcgacatctctggttctaaccttagctaccaaacaggagatcatatcgccatctggccgactaacagtggtgatgaagttgaccgttttctggatatcataggtttgaaggagaagaggaactatgtgatctccgtcaaagcattggaacccacagctaaggttccttttcccaccccaactacgtacgatgcgatcgtccgttaccatctggagatctgtgcaccggttagcaggcagtttgccgccacccttgccgctttctccccggacgaagaggtgaaggccgaaatgactaggctgggttccgataaacagtatttccacgataaaattggaccccacttttataatatcgcacgttttttagaagctgtagggaaagggcagaagtggacgaacattccgttttccgctttcattgagggattgaccaagctgcaaccgagatactactccatcagctccagttcattggtacagcctaagaaaatctccataacagccgttgttgagtcacgtaaaattccaggaaggaatgacccctttaacggggtggcaactaactacttgttggccctaaaacagaaacaaaatggtgatcctaacccgtctccattcgggaagacgtatgcacttaacgggccaagaaacaaattcgatggaatacatgtgccagtgcacgtaagacacagtaactttaaattaccatcagacccgggaaaacctgttattatggtcgggcccggtacgggtgtcgccccattccgtggcttcattcaagagagagccaagcaggcccaaaacggcgcgacggtcggtaagaccattctattctttggctgtcgtaagagaagcgaggattttttatatgagacagaattcgaagaatacaaaaaggctttaggagattctttggagattgtgacagcctttagtcgtgaatcttctaagaaagtgtacgttcagcatagattaaaagagagatcaaaagaagtcggggaattacttagccagaaagcatgtttttatgtatgtggggacgctgcgcatatggcaagagaggttaacactgtgcttgcccagatccttgctgaatctcgtggcgtgtcagaagctaaaggcgaggagatagtaaaaaatatgcgtgccgcgaaccaatatcaagaagacgtatggagc

SEQ ID NO: 66 - DNA, 2319 bp, synthetic construct
  source 1..2319: mol_type = other DNA
atggcagccgcgcaattggacaccttagacattattgtcttagcagcgatttgccttggcacggtagcctactttacaaaagggacgtattgggggattacacaggacccctacgcttcaacgtattccgcggctaattcatcaaaagccgggaaaactcgtaatatacttgagaagatggaggagagcaacaagaattgcgtggtattctatggctcccaaacgggcactgccgaagactacgcgagtagactagcgaaggaggggaagagccgttatggtttagaaacaatggttgctgatttagaggattatgattacgagaaccttgattcttttcccgaagataagatcgctatgttcgtcttggccacttatggagaaggtgagccaacagacaatgccgtcgatttttacgaatttatcaccggtgagagtccctcattttcaaatgagtccgagcctccgttagggaatctgaagtttgtggctttcggtctagggaataacacctatgagcattacaacagtatggttagagacgtgactaaggcactagagaagctgggcgctaccaaaattggtgcggcgggagagggagatgacggagccggcacgatggaggaagactttctagcctggaaagacccaatgtgggttgatcttgctgaaaaaatgggactggaggagcgtgaagcagtgtacgaacccgtctttgccatcactgaaagagagggcttgactaaggatagcccggaggtctatctaggcgagccaaacaaaatgcacttagagggggctagtaaaggtccttacaatgctcataatccttacatcgcgccgattgcggagagccgtgagatcttcacagtcaaggataggaattgtctacatatggagattgatattagcgggtcaaacctatcttatcaaacgggtgatcacgttgcggtttggccgacaaatgctggccgtgaggtcgacagatttctaaaagtcacaggccttttgccaaagagggatactgtggtcggtgtaaaggcgctggaccctacagcgaaggtcccgtttcctactcctactacttatgacgccattgtacgttaccacatggagattggggccccagtcagcaggcaattcttagcgactctggctcctttcgcgcccaccgaggctgcaaaagcggagatggccaaattggggtcagacaaggactatttccacgaaaagattagtaatcaatacctaaatatcgcccaggtgcttcaagcagttgccggggatgagaaatggacatccattccgtttagcgcatttattgaaggcgtgaataaggttcagccgaggtattactctatttcatcttcttcactagtacaaaaaaagaaaatttcagtgactgcgatagtggaaagtatgcatattcctggtagagaggatgccctgaagggagtcactactaattatcttcttgcacttaaacagaagcagaacggcgacccaagccctgacccgcatggacttacttacgatattacgggccctagaaacaagtacgatggcgtgcacgtgccagttcatgtgaggcatagcaattttaaattgccctccgacccctcaaagcctattataatggtaggcccaggcaccggtgtggcgccctttagagctttcgtgcaggagagggcacagcaggccaagaacggcgagaacgtgggtaggactttgcttttttttggatgccgtaaaagtactgaggatttcctgtactccgacgagtgggaaacatataaaaaggaccttggggataagtttgaactgataacagcgtttagtagggaaacaccaggaaagaaggtgtatgttcagcacagactgagcgaaagagcgaaagaagtgaatgagcttctacaacagaaggcttacttttacgtatgcggagacgcagcaaatatggcccgtgaggtaaacaccatattgggaaaaataatagcggaacagaggggcatacctgagacgaaggcggaggacatcgttaaaggcatgagaagtgccaatcagtatcaagttagcagccttaactcaacctctttcgaacctacgagcacgctgctgactgcccccgggtctagtgttagggatttcgattgtgacatcatgtttccgatgtatcacaggtcatggataggaccttgtacgatccctgcctatactagccagacggatgcagatttcaactttttagggggatgtgtcgtaatgaatactaagtccttattacttagatacgatgactccacattcattttctgggga

SEQ ID NO: 67 - DNA, 2082 bp, synthetic construct
  source 1..2082: mol_type = other DNA
atggcagccgcggaatttgacactctagacattgtagttctaggggtaatcttgctgggaactatcgcttactttacaaaggggactctatgggggataactaaagatccgtacgctaatgcgttcgcgaatgcaaacggggtgaaagcggggaggtcacgtaacattttagagaagatggacgagacggggaagaattgtgttattttctacggtagccagaccggaacggcggaagattatgcgtccagactagccaaggagggaaaatctaggtttggtcttgagacgatggtagctgacctggaggagtatgacttcgacaatctagatgcaatgccatccgacaaaatcgtcatgttcattctagcgacatatggggagggggagcctactgataatgccgtcgaattctatgagtttatcaccggtgaggacgtttctttcagcgaaggttccgagcctgccctacagaatcttaactacgtggcattcggtttgggtaacaatacctacgaacactacaactccatggtgcgtaatgtggataaagcattacagaagctaggggctaatagaataggggaagctggagaaggggacgacggcgcaggtaccatggaggaggactttctggcgtggaaagatcctatgtgggccgccttagccgagaaaatgggcctggaggagcgtgaagcagtttatgaacctacgttcgggatagtggatcgtgagaatctaaccgttgactctcctgaggtctatttaggagagcctaacaagatgcacttggagggtacagccaaaggtccgtttaactctcataacccttacatcgcgccgattgcggaatccagggaattgttttctgcgaaagacagaaattgcatccatatggacgtagacataagcgggagtaacctatcatatcagaccggtgaccatatcgcgatctggcctaccaattccggagatgaggtggacagatttctggatatagttggtctgaaggagaaacgtaacaacgtgatatctgttaaagctctggaaccgacggccaaggttccttttccaacccctacgacctatgatgcaatagttagatatcatttggaaatctgcgcacccgtaagcagacaatttgtggcgacgttagctgccttcgcgccgaacgacgaagccaaagcagagatggctaggttggggagtgacaaggactacttccattccaagacagggcctcacttttacaacatcgcacgtctattagatacagtaggcaaaggcgaaaaatggacgaacattcccttttctgcgttcattgagggtttaaataaacttcagccccgttattactctatcagttcctcctcattagtccaacccaaaaaaataagcataacagcagtcatcgagagtcaagcaattcctgggagacaagaccccttcaggggcgttgccactaattacttgttcgccctaaagcaaaaacagaacggtgaccctaacccttctccctttgggaaaacctatgccctgaatggacctagaaacaagtttgatggaattcacgtcccagttcatgtaagacactccaattttaagctaccatctgacccggcgaagccggtcattatggtgggcccgggtactggcgtggccccgtttagaggttttattcaagagagggcacgtcaggcccagaacggggcaactgttggtaggacaatactttttttcggttgcagaaaacgtagcgaggattttttatatgaatctgagtgggaagaatataagaaagcgttgggggatagcttggagatcgtcacagccttttccagagagagcagtaaaaaagtttatgttcagcataggttaaaagagaggtcaaaagaaattggggagttgctttcacaaaaggcatacttttacgtatgcggagacgccgcccatatggcgagggaagtaaataccgtccttgcgcagattatcgcagaatctcgtggagtatctgaaaccaagggcgaagaaatcgtgaagaacatgcgtgcggcgaaccaataccaggaagacgtttggagt

SEQ ID NO: 68 - DNA, 2091 bp, synthetic construct
  source 1..2091: mol_type = other DNA
atggcagccgcccagctagatacacttgaccttgtggtcctggtggccttgctggtaggaagcgtcgcttactttaccaagggatcatactgggcagtccccaaggacccttatgcaagcagcgccagtacgttgaatggggccgcaaaatccgggaagacgagagacatagtggaaaagatggatgaaacgaataaaaactgcgtgatcttttacggaagtcagactggcacagcagaagactatgcgtctagattagccaaggaaggttcccagaggttcgggctgaagacgatggtggcagacatagaggattatgactacgaaaatttagataagtttccggacgataaagtagctttctttgtcctggctacctatggggagggcgagccgacggacaacgcggtcgaattttaccagttctttacaggcgaagatgttgccttcgagtccggtgcatctgcagacgacaaacctttgagttctctaaaatacgtcacgttcggtctagggaataacacatacgagcactataacgccatggtccgtcaagtggacaccgccttaacaaagctgggcgcgcaaaggatcggtactgctggtgaaggcgatgacggcgcagggactatggaggaggacttccttgcatggaaagaaccgatgtgggccgcgctgagcgatgcgatgaatctgcaggagagggaagcggtatacgaagcagttttctccgtagtagaagatgacgaaaagtcccctgaggaagaaagcgtgtacttaggtgaacctaccgccagtcacagggaaggtcgtgccaaaggaccattctctgcgcacaatccgtacgtcgcgcctatcgtagaatcctctgagttattcacagttaaagaccgtaactgtcttcatatggagatatctattgcgggctccaatctgagctaccaaacaggggaccatatcgctatatggccgaccaacgccggtgcagaggtggatagattcttacaggtcttcggcattgaggagaagagacacagtgtcataaacatcaaaggtatcgacgtgacagcaaaagtcccgataccgacccccaccacatacgatgctgcggtgaggtactatatggaagtatgtgctccggtttcaagacagttcgtatcaactctggctgcttttgcacccgacgaggattctaaaacagagattgttcgtcttgggaatgacaaagactactttcatgaaaagattaccaatcaatgcttcacaattgctcaggccttgcaatcaatcacaagtaagcccttcacggcggtcccgttctcccttttgattgaagggttgaacaaattacaaccgcgttactattccatttcttcttctagtttggtccaaaaagacaagatatccatcacagcagttgttgaatcagtccgtcttccgggcgcatctcacatcgtcaagggtgttacaacgaactatctgttggcactaaaacaaaagcaaaacggagatccaagtccggacccacatggtttgacatattcaataaacggacctcgtaataaatatgatgggatacatgtcccagtgcacgtccgtcattccaatttcaagctgccgtccgatccaagcaaacccataatcatggtgggcccaggcacaggcgtggcaccattcaggggatttatccaagagcgtgcagcacttgcagccaaaggagaaaaagttggaacaacgttgttgttcttcggatgtaggaagtccaccgaggacttcatttacaaggatgaattcaaaacatttcaggatcaaatgggcgactctttgaaaattattacagcatttagtagggaagggagcgaaaaggtgtacgtacagcaccgtcttaaagagaacgcggagcttgtcagtgagttgcttaagcagaaagctaatttctatgtgtgtggtgatgcggctaatatggcacgtgaagtaaatctggtgttaggtcacatcattgcgcagcagagaggtctaccggctgagaagggggaagagatggtgaagcacatgagatcatccggatcatatcaggaggacgtgtggtca

SEQ ID NO: 69 - DNA, 2091 bp, synthetic construct
  source 1..2091: mol_type = other DNA
atggcagccgctcagctagacacactggatctagttgtcttagtcgcgttgcttgtaggaagtgtggcgtatttcactaagggttcatactgggcggtccccaaagacccatatgcatccagtgcggcgactcaaaatggtgctgcgaaaagcggtaagactcgtgatatagtagaaaagatggaggagacaaacaagaactgcgttatattctatggatctcagacggggacggcggaggattacgcttctcgtttagctaaagaggggtcccaacgtttcggtttgaagacaatggtagcagacatcgaagactacgactacgaaaacttagacaagttccccgatgataaggtagccttctttgtattagctacatatggcgaaggggaacctaccgataatgccgttgagttttaccagttcttcactggcgaggatgttgctttcgagagcggcgcctctgctgaggacaaaccactttcttcccttaagtacgtgacgtttggtttaggaaacaacacttacgaacactataatgcgatggtaaggcaagttgacgcggcgttgaccaaacttggtgctcaaagaattggaacggcgggcgagggggatgatggagcgggcactatggaagaagactttctagcctggaaggagccaatgtgggcagcactgagtgaagccatgagcttgcaagagagggaagcagtatatgaggccgtgtttagcgttgtcgaagacgacgagaagtctcccgaggacgagagtgtctatttaggcgaaccaacggctagccatagggaagaccgtgcgaaggggccatttagtgctcaaaatccctacatagctccgatcgtcgagtccagtgagttatttactgtgaaggacaggaactgtttacacatggaaatcagcatagcgggaagcaatctatcttatcagactggcgatcacatagccatttggccgactaacgccggcgctgaagtcgataggttccttcaggtttttggactggaagaaaaacgtcactctgttatcaatgtgaaggggatagatgtgactgccaaggtgccgataccgacgccgaccacctatgatgccgcggtgagatactacatggaagtgtgtgctccggtaagtagacaatttgtaagctcattggccgcttttgcacctgacgaagagtcaaaagtggaaatggtcaggttggggaacgacaaggactattttcacgaaaaaatcgcggaccagagattcacgattgcgcaggcacttcaatcaatcacaagtaaaccattcaccgcagtaccattctccttattaattgaggggctgaataagctgcaaccacgttattattctatatcaagcagtagcctagttcaaaaagataaaatatcaatcacagcagtagtggagtccgtacgtttaccgggagcaagtcacatggtcaagggtgttaccactaattacttacttgcgttgaaacagaaacagaatggtgatccctctcctgacccccacggccttacgtactcaataacgggcccccgtaataagtatgatggcattcacgtcccagtacatgtccgtcattctaattttaaattgccttctgaccccgcaaaacctattatcatggttggtcccggcactggcgtagcgccctttagaggcttcatccaggaaagagccgcgctggtcgctaaaggcgaaaaggtcggaactacattattattctttggttgtcgtaagagcgatgaagatttcatgtacaaggatgaattcaaaacgttccaagaccagatgggcgattcattaaaaataatcacagctttcagtagggagggcgcagaaaagatatatgttcagcatcgtttaaaggaaaacgcggaacttgtgagcgacttgcttaagcagaaggctaatttttatgtttgcggtgacgcagctaacatggcccgtgaggtaaatctagttctgggacacataattgcgcaacaacgtggccttccggccgagaaaggtgaagagatggtcaagcatatgcgtagttctgggtcttatcaagaagacgtctggtcc

SEQ ID NO: 70 - DNA, 2082 bp, synthetic construct
  source 1..2082: mol_type = other DNA
atggcagccgctgagttagatacgttggatttggtagttttggcggcaattctgctgggaactcttgcctactttactaagggtaagtactggggtgtggtgaaggacccctatgcgacagcatttgcgaatgccaacggcgtgaaagccgggaggactagaaatattgtggagaagatggacgagagcggcaaaaactgcgttatattttacgggtctcagactggtacggccgaagactatgcgtcaagacttgctaaagagggcaagagtcgtttcggacttgaaactatggtagccgacttagaagagtacgacttcgataatttagatacagtaccgactgacaaggtagtgatgttcgttctagcgacatatggtgagggggagccgaccgacaacgcggttgatttttatgagttcatcacagcagatgatgttgcgttatctgagggctccgacccggccctgggcaatctgaactacgtagcctttggcctaggtaacaatacatatgaacactataatagtatggtaagaaacgttgataaggcgttacaaaaacttggtgctcacagaataggggaggctggggaaggagacgacggggctggcacaatggaggaagacttccttgcgtggaaggaccccatgtgggctgccttagcaagtaagatgggactggaagagcgtgaagctgtttatgagcctacttttggcattgtagatagagaaacactgacgagggattctgtcgaggtgtaccttggcgaaccaaataaaatgcacctagaggggaccagtaaggggcctttcaacgcacataatccatatatagcccccatagcagagtccagggaattatttagtgcaaaagaccgtaattgcatccacctagacatcgatgtgtccgcgagtaaccttaactaccagacaggcgaccacatagcggtgtggccaacgaatcctggagacgaagtagaccgttttttagacgtgatcgggttaaccgagaagcgtaacgatgtaatctctgtgaaggctttggagcccactgcaaaagtgcctttccctacgccgacgacgtacgatgcaattgtacgttaccatttggaggtgtgtgcccctgtgtccagacagttcgtagccactcttgctgctttctctccctcagatgatattaaggccgagatgacaagattaggcaatgacaaggactatttccacgataaaacaggtccacatttttataacatagcacgtttgcttgcaactgtggggaagggccagaagtggactaatataccgtttagcgcattcatcgaagggcttaataagctgcagccgaggtattacagcatctcttctagctctcttgtacagcctaaaaagataagcattacggcggtcgtcgaatcacagatgatacctggtagaaatgaaccgttcagaggcgtggccactaattatctgtttgctcttaaacaaaagcaaaatggtgacccaaatccccaaccattcggacaatcctacgagctgaacggcccgaggaataaatacgatggtgtgcacgtcccagttcatgtcagacactcaaatttcaagttgccgtccgatcctggtaaaccggtaataatgataggcccaggaacgggcgtggctccgttcagaggatttgtgcaggaacgtgcaaagcaggcgcgtgagggcgcgaatgttggccgtacgttattattttttggctgtagaaaatctactgaagacttcatgtacgagtcagaatgggcggagtataagaaggatctaggcgacaaattcgagatggtcactgcattctctagggaaacaaataagaaagtatatgtgcagcaccgtttaaaggagagagcgaaagaggtcaacgaattacttcagcagaaagcctttttttacgtatgcggagacgcggctaacatggctagagaggttaatgccgtgttggctcagattatagcggaaggcaggggagtgagtgaacagaaaggggaagaggttgtcaagcacatgagagctgctaatcagtatcaggaagacgtttggagt

SEQ ID NO: 71 - DNA, 2088 bp, synthetic construct
  source 1..2088: mol_type = other DNA
atggcagccgcacaggttgatactcttgacatgttagtcttggcggtaatcctggtcgggactattgcatattttacgaaaggaaaatactgggccgtagtgaaggatccatacgcaagctcctatgcagccacgaatagtgctaagcagggtcaaacaaggaatatagtagagaaacttgaggagaccgggaaaaattgcgtcattttctacgggagtcagacaggcacggcggaagattatgctagcaggcttgctaaagaaggctcccagagattcggcttgaagaccatggttgccgatttagaggactatgattacgcgtgtctagacaagttcccggaggacaaggttgcttttttcgtacttgcgacatatggtgaaggagaaccgacagacaacgccgtggaattttttcagttcataacgggtgacgacgccagcttcgaagatggaggaagtcccgaggaccaaccactgtctaaaatgaggtacgttgcgtttggactaggaaacaatacatacgagcactacaacgcaatggttagaattgtcgataaagctcttacgaaatgtggtgctaagagaatcggagacgcgggtgagggcgatgatggagcagggactatggaggaagattacttagcgtggaaagaaccaatgtggaaagccttggcggaggagatgggccttgaggagcgtgaagctatctacgaaccgacttttagcgtaatcgaagaagcagaccttaacaccgaatccgaaaatgtttacttaggtgagcccaactcctcccatttgaaaggaagaggtcagggaccttataacgcgcacaacccgttcttaagccctatcgttgaatcccgtgaactgttctccgttaaggatagaaattgtctacatatggagttcgacattaaagggtcacaattaaattatcaaacaggggaccacatcgcaatctggcctactaatgcaggtcaagaagtagacaggttcttaaaggtgtttggtcttgaggagaagagacacacggtcgtaagagtgaaaccgatagacgtaacagcaaaagtgccctttccgcagcctacgacctacgacagcgtagtcaggtattacatcgagacttgcgggcctgtaagtcgtcagtttatcagccagctggcggcattcgcacccgatgaggagacaaaaaagaaaatgataaggataggcgaggacaaggatgtcttctccgataaggtatctgcattttatttcaatatcgctcaagccctgcagtcctttacggacaaaccgtttactgctgtcccattttctctattgatcgagggcattccaaagatccagcctcgttattattctatctcatccagtagtatcgttcagaaagaaaaaatctctatcacagcggtcgtggagtcactgcgtgtacccggcgcggggcacgtcgtcaaaggtgtaacaactaactacttacttgcattaaaacagaaacaacatggggaacccaacccggatccttttggtctgacctacgctatcagcggaccacgtaataaatatgacggcttccacgttccagtacacgtaagacattccaattttaagttgccttccgatccgtccaaacctattattatggtgggaccaggtacgggtgtagctccgtttagaggtttcatccaggaaagggtacatcaagccgagaaaggtgagacagttggccccaccattctattctacggatgtcgtaagtccacagaggattttttatataaagaggaatttgaagaggtaagtaagaagttaggtgactcattcaagttgataacagctttctcacgtgagacatcacagaaagtgtatgtccaacacagattaaaggaacatgcagagctagtaagcgatctgttgtctaaaaaggccaacttctacgtttgtggtgacgccgccaacatggcccgtgaggttaacgtcgtactgggtcagatcttggcagagcagaggggcctaaaacctgagaaaggagaggaactagtaaagcatatgaggaacacaggcagctaccaagaagatgtatggtca

SEQ ID NO: 72 - DNA, 1173 bp, synthetic construct
  source 1..1173: mol_type = other DNA
atggcagcccgtatactgaagtcacaccctttacttaagatagttaattcatatatcattgatagcccacagcccgcgaatatatcctatttatggaatttcgggtcattacttgctttatgcctgggcatacaaatcgtaacgggtgtcactttggccatgcattacacgccctctgtgcttgaagcgttcaattctgtcgagcacattatgagggatgtcaataatggttggttagtgagatatttacatgctaataccgcctccgcttttttcttcttagtatacctgcatataggaaggggcttgtattatggctcttataaggcgcctaggacattaacctgggcgatcggaacggtcatcttaatagtaatgatggccacggcatttttggggtacgtccttccatatgggcagatgtccctgtggggggcgacagttatcactaacttaatgagtgcgatcccatggatagggcaggatattgttgagtttatatggggtgggtttagtgtgaacaacgcgacgcttaaccgtttcttcgcgctgcatttcctattaccattcgtacttgccgctctggtgttaatgcaccttattgctatgcacgacactgtcggctctggtaaccctcttggcatcagcggaaattacgaccgtttacctttcgcaccctatttcatcttcaaggacctggtgaccatcttcattttttttatagtgttatctatctttgtgttttttatgcctaacgctctgggggatagtgagaactatgttatggccaatcctatgcaaacgccaccggctatagttcctgaatggtatttactgccattctatgcaatcttaaggagtatcccaaataaattgttaggtgttatcgctatgttcagtgccattctggcattgatggtgatgcccatcacggatctttccaaattgaggggggtgcagtttaggccgctaagtaaagttgcgttttacatctttgtggctaattttttggtgctgatgcagctgggagctaaacacgtcgagacgccatttattgaactgggccaaatatcaacggtgttgtacttcgctcatttcttcgtaattgtgccagtagtgagcttgattgaaaactctcttgtagaattgtctacaaagtacaactcctcatgt

SEQ ID NO: 73 - DNA, 1233 bp, synthetic construct
  source 1..1233: mol_type = other DNA
atggcagccagaatcctaaaatcacatccgttgttaaagttagcgaatgcatacctggttgaccattcccagccaagtaatatctcttattggtggaattttggaagtctgttgggcctttgcttgcttatccaaatactgacaggggtgttcttagcgatgcattacaacgcatctatcgcggaagctttcaactctacagaacacattatgagagatgttcaaaacgggtggataatacgttatttgcatgccaacactgcttcagccttctttttctgcgtatacatgcacttgagcagggggctattctacggcagctaccgttcaccgagaacgctagcttgggtcatcggtacgattattcttatattaatgatggcgacagcctttctgggatacttgtacgtattaccctttggtcaaatgtctgccaccgtaataacaaacttgttgtccgcaatcccatggattggacaggatgtggttgagtttttgtggggtggattttcagtgaataatgccaccctgaaccgtttctttgcgctacacttcatcttaccattcgtgctagcagctcttgcgattttacatctgatagcggtccacgagacagcaggggcatccaatcctcttggagtaccgggatattatgatagagtacctttcgccccgtacttcatatataaggatctaatcactatattcttgttcttttttattttgagtgtcttcgtgttttttctgcctaaccagctgggcgactgcgagaattatgtcgtcgcaaacccaatgcagactccagctgcaattgtgccagaattgcttccgttttacgctatccttcgtagcatccctaacaaattattgggcgtaatagcgatgttcgctgccttagtgagtatcttggtattgccgtacagtgacattggccgtacaaagggccttcagtttagacccttgagcaagctgatattctttatgtttgttgccaatttcctgttactgctacaacttggtgccaagcacgtggaatctccattcattgaattagggcagatcagcacggctttttatttcgcatacttttttcttctagtgcctagtgtgtctatattagagaacactctactggacataaaaacgtatgcgctgaacaacaaaaaaaagatcgtcttgtatacggtgtatactttaatcgctaaccgtatgtttaaggtaaggctgctggca

SEQ ID NO: 74 - DNA, 1167 bp, synthetic construct
  source 1..1167: mol_type = other DNA
atggcagccaggatcttgaagagtcacccattcttgaagctgctaaatgcctatttgatagaccactcccaaccttcaaatatttcttacttatggaatttcggctccttattgggattgtgcttaggtattcagattatcacgggaataacgcttgctatgcactataatccgtctgtagctgaagcattcaacagtgtggagcatatcatgagggacgttaacaacggatggctagttaggtatctgcattctaacactgcctctgcattttttttcctggtttatcttcatattggaaggggtctatattacggaagttatcgtgctccgagaaccctggcttgggtactgggggcaataattcttatccttatgatgggtacgggattcttaggttatgtgttaccctatggccagatgtctctgtggggcgctacggtgatcactaaccttattagcgccattccttggatcggacaggacattgtcgaatcccacaaatgtagcgtcaataatgctaccctaaatcgttttttcgcactacatttcgtgttgccatttgtccttgcggctcttgcactgatgcaccttatagctgttcacgagagcgcgggcgcatctaatccactgggagtcccagggtattatgatagggtccctttcgcgccttactttctattcaaagatctaataacaatttttatcttcttctttgtcctttctatgtttgtgtttttcatgcctaacgtgctgggtgatagcgaaaactatgtggtcgctaatcccatgcagactccagcagccattgtaccggaatggtacttattacccttttatgccatccttcgtagtatccctaataaattgttgggtgtaatagctatgtttgccgcccttgtcgcaattttggcgctgccctacacagatttgggtagaacaaagggactgcaatttagacccctttctaagattctattttatatcttcgttgccaatttcttgatacttcaacagcttggggccaaacacgtggaaactccattcatcgaatttggtcagatcagcactgccttgtacttcgctcacttctttatactggtccctgtcgtatcagtcatcgaaaataccttaatagatttataccagattaataacaaatcccgt

SEQ ID NO: 75 - DNA, 1170 bp, synthetic construct
  source 1..1170: mol_type = other DNA
atggcagccagaatctttaaatcccatccactgttgaaactaattaacagttacattattgactcccctcaacccgctaatctaagttacctgtggaattttggtagcttacttgcattctgccttgccattcaaatcgtcacgggggtcacgttggcaatgcattataaccctagtgtgttggaggcgttcaattcagtggaacatattatgcgtgatgtaaacaatggctggttgatcaggtatcttcatagcaacactgctagcgcctttttctttcttgtctacctgcatattgggcgtggtctatactatgggagttacagagcgccccgtacattggtttggacgataggcacagtcatctttatcctgatgatcgtgactgcctttctagggtatgtgctaccgtatggacagatgtcactgtggggggcaaccgtgataaccaacctaatgagtgcaattccctggatagggcaagacatagtagagtttctgtggggaggcttcagcgttaacaatgctacgttaaacaggttcttcgcactacacttcgttttgccgttcgtattagccgcactggttcttatgcatcttatagcccttcacgactctgcagggtccggaaatcctcttggtgtctctggcaattacgacagacttcctttcgcgccctatttcctttttaaagacctggttacgatctttctattcatactaattctttcagtctttgtattttttatgcccaacgtccttggcgactcagaaaactatgtggtagctaatccaatgcaaaccccacctgcgattgtccctgaatggtatctgttgcccttttacgccatccttaggtcaattcctaataagttgcttggagtaatcgctatgttgagcgcgatcttaatgctgttaactatgcctttcgcggatctgggtagaactcgtggtatccagtttagaccattgagcaagatggctttctacatattcatcgcaaactttttaattttgatggtcttaggggccaaacatgtcgaatctcctttcattgagtttggccagatctcaacggttatctattttgcgcattttcttataatagttcccttcgtgtccttactggagaacagtcttatagaattgtcaaccaaaaaggcacgtaat

SEQ ID NO: 76 - DNA, 1176 bp, synthetic construct
  source 1..1176: mol_type = other DNA
atggcagccaagataattaagaaaagtagtttcttcaaagtcgtattgaatgcctatcttattgaccattctcagccctccaacataaactacctatggaacttcggctcactattaggtttatgcctgggtattcaaataataacaggtgtgacgttagcaatgcactataatcctagtgtagctgaggcgtttaattctgtagagcacatcatgagagatgtcaataacggatggttggttcgttatttacacagtaatacggcttctgcattttttttcctagtttatttgcatatgggaagagggctatattatggctcttatagggccccgcgtactctagcgtgggttatcggtgctattatactaatacttatgatggggactggtttcttaggatacgttttgccgtatgggcaaatgagcctatggggagcaacggttataacaaatttaatctccgctataccttggataggccaagatatcgtcgagttcatatggggaggcttctctgtgaataatgcaacgttgaatagattctttgctctgcattttgtactgccgttcgtgttggccgcactggcgttgatgcacctgatcgcagtgcacgaaactgcaggcgccagtaacccacttggtgtgccaggttactatgatcgtgtacccttcgcgccgtatttcttgtttaaagacctaattaccatttttatttttttttttgtgttatctatgtccgtcttttttatgcccaatgtgttaggtgatagcgaaaattacataatggccaatcctatgcagacccccgccgccattgtcccagaatggtatctacttcctttttatgctattcttagaagtattccaaataaattattgggggtagtcgcaatgttcgcaagcctggtagcgattctagccctgccattcaccgatttaggcagaacaaggggactacaatttaggcctttatctaaaatcctattctacgtgctactggcaaacttcctaattttgcaacaactgggtgctaagcacgtggagagcccattcatcgaatttggtcaaatatcaacggcgctttattttgctcattttttcatcttggtcccggttgtctccgtgattgaaaataccttgatggatctttaccagacgaataataaatcaaga

SEQ ID NO: 77 - DNA, 1161 bp, synthetic construct
  source 1..1161: mol_type = other DNA
atggcagccaggatactaaaatcacacccgttattacgtttggtcaatagttatttgattgacgcgccaacgcctgcaaacatatcatacctatggaattttggaagccttctggctatttgtctaattatacaaatagtaacgggtgtaacattggcaatgcactatacaccttcagtcgccgaggcgttcaatagcgttgagcacataatgagggatgtgaacaatgggtggctaatcaggtacttacacgcaaatactgcctctgctttctttttccttgtctatcttcatattggcagagggttgtactacgggagttacaaaagtcctcgtacgttaacctggatcattggcacggtgatactaataatcatgatggccaccgcattccttggttacgtgcttccgtacggccagatgtccttatggggggccacagtcatcaccaacctaatgtcagcgataccttggatcggacaggatatcgtggaattcatatggggcgggttctctgtcaacaatgcaactttgaacagatttttcgccctgcactttctgctgccatttgtcctagccgcactggtactaatgcatttgattgcctaccacgacgtagtggggtctggaaatcctctaggcatttctgcaaactatgatcgtcttccctttgccccctattttctgtttaaggacctggtgactatattccttttttttattgttcttagtatctttgtgttttttatgcctaacgctttgggagattctgagaattatgttgtggcgaaccccatgcaaacgcccccggcaatagtaccggagtggtatctgctacccttctatgctattctgagaagtatccctaacaaattactaggagttatagctatgtttgctgccattctagctttactggcgatgcctctgacagatttgagtaaacttcgtggggtacaattccgtcctctgtcaaaaatcgcattcttcattttcgtggcaaatttcctgatccttatgcaaataggagccaaacatgttgagacgcccttcatagaagtaggacagatcagtacggttttgtatttcgcccacttttttatcatagtgccggtggttagtatcatagaaaactccttggtggaactagctacgaagaag

SEQ ID NO: 78 - DNA, 1173 bp, synthetic construct
  source 1..1173: mol_type = other DNA
atggcagccagattactgaaaagtcacccgttcctgaagctggtaaatagttatttgattgacgccagccagccgactaatatcagttacatgtggaacttcgggtcccttctggcagtttgcttgggcatacaaatcattactggagtgactttagcgatgcactacaatccttcaattgcggaagctttcaacagtgtggaacatattatgcgtgacgtgaacaacggttggctaattagatacttacactcaaacactgccagcgcgttttttttcttagtctatctgcacataggccgtggcttttattatggaagttaccgttctccgcgtagcttaacttgggttattggaactataattctaattcttatgatgggtatcggattcctagggtacgttttaccgtatggacaaatgtcactgtggggtgccacagtcattacaaatcttatttcagcaatcccatggataggtcaagatgtcgtcgaatttatctggggtgggttctctgtgaataacgctactctaaatagattcttcgcgttacatttcgtcctaccttttgttttggcagcactggtactgatgcatcttatcgctttgcacgatacggcgggctcatctaatcccctgggcactccaggcaattacgacagaattcccttcgccccgtactatctttttaaagacttgattactatcttcatattcatatttgtattgtcaatttttgttttctttatgccaaacgtgctgggcgactccgaaaactacataatggcgaatccgatgcaaacccctgcagcaattgttcctgaatggtatctacttcctttctacgctattttgaggagtatacccaacaaactacttggagttatcgccatgtttgctagcctacttgctttattggccctacctaaagcagatcttggacttacgaaagggttgcagtttagaccgctttcaaagatagtgttttacatctttgttataaattttctgattcttatgcagttgggggccaaacatgtagaatccccatttatcgagttcgggcagatctctacggcaatatactttggctattatttcgttatcatgccggctgtttctgtgttggaaaatacattgattgatttatatcaagctaataataaggccata

SEQ ID NO: 79 - DNA, 1224 bp, synthetic construct
  source 1..1224: mol_type = other DNA
atggcagcccgtatatttaaatcacacccactattgaagttggtaaactcttacattattgattcacctcaaccaaccaatctaagttatttatggaactttggcagtttattagcactatgccttggaatccaaatagtgaccggggtaacattagctatgcattactctcccagcatcactgacgcctttgacagcatagaacatatcatgcgtgatgtaaacaatggttggttaatccgttatcttcatgctaatacagcctccgctttcttctttcttgtctatttgcatatgggtcgtgggatttactatggcagctaccagggctccaggtctttaacgtggaacataggggtagtcatcttcatagtcatgatagtaactgcatttcttggctatgtattgcctttcgggcagatgagcctgtggggcgctaccgttatcacgaatctattatccgcgattccatggataggacaagatctagtcgaattcatctggggcgggttttctgtaaataatgccactctgaacagattttttagcttacactacttattgcctttcatccttgctgccctagttctaatgcatctgatagccctacatgacactgtaggttcatcaaatcctctgggaattagtggaaattatgacaggcttcccttcgcgccttattttctgtttaaagacctagttacgatattccttttcatgttgggactgtctatatttgtgttctttatgcccaatgctcttggagactgtgagaattatgttatggctaatccaatgcagacgcccgccgcgatagttcccgagtggtacttattacctttctatgcgattcttcgttcaatccccgataaaactcttggggtagtagctatgttgggtgccattttgatactgatggctcttccatatctggacaaaagtaatatcaggggtatgcaatttaaaccactatcaaagatcgcattctacatttttattgctaactttttggtcttaatgatacttggcgcaaaacacgtggaagatccgtttattatattcgggcaaatcagtactactctgtacttctcatatttcataattatcactccgttgttctccatctttgacaacattctattcgacattgctacgaagaagaacaatgaaatagtcaataaatactacaacattgttataaaaaaggatattctgttgcctgag

SEQ ID NO: 80 - DNA, 8454 bp, synthetic construct
  source 1..8454: mol_type = other DNA
atgtcatccaaagcgggaggtgccttttgcgccctagatcctttctatccagttagcagattgacatatatgtgtcaaaagctggagatttccattgttttgactacccagtccaatttggaacaagcgagccttttgcccggtacagtattcgtcttgggggatgacctttacacggagagtgcatacgcgggtgcacaacaaccacatttatggagcgacgtgagccctaacgacccggtatatgtagtctttacttctggtagcacaggtaatcccaagggaatcataatggagcatcgttctttctcttcatgtgccttatctgcaatggaaccgttgcatattcttcctcaggataggattctacactttccatcatatgccttcgatataagtatatttgaaatactaactccactagtggctggcgctacggtcggaattccgtcagaaaaagcccgtcgtgagaatctgccacacgctataacagaactgggagctacgtgggcgtttttaacccccaccgttgcgcgtatgtaccgtccaatgcaggtgccatctcttaaaacgttatgcctgggtggagaagcgatatctgcctcagacatagatctgtggctttctaaaaaccttataaccggatatgacccggccgaatgctgtcctttagcttcctggatagtcgatcctaaagatcacaggaaattgcttcccgtaggtgcaatcggagagttggtaattgaagggcccaatgttgcaagaggatacattcatgaccctacatgttctgcgtcagatactccgtttttggttacaccgccaccttggctgtccagattcaggactaatttcgatagagggtcaagactattccctactggagacctggtgaggtacgacgacgaagaagcggtccactttatggggagaaaagacttacaggttaaaattaacggccaaagggtggagcttgctgagattgagtatcacttagatcagcattttcaactgttagctgcaaaggtcatagtggatactttacctgtctcacgtagaactatattaacggcactaatccttcccgaccaaccactgaagaacgacgaggagcctgggaaatctttattgatggagttacgtgaagtgcatcaagactttcaagcaaaattggcgtcagtagccagtaaattacaattgaccttacctaaccatgtggtgccgagcgtctatctacctattagaagagtgcctatgtcagagtccggcaagatagacagaagacgtttaaaaagtttaatcctatccttgagccaggaagacttatatgggtctaggaaaccaatgcaaccgggggaagagcctgcaagcgacaccgagaagcttttgaggtcattatttgcccagaccttggacctaccccatgccagaataaagagtgactctaacttttttcaactaggaggcgatagtgtcttagcaataaaattgctggcccttgcactggaacagggaattaaatccatagcctatcaagatatcttcaaccaccccactctgcgtaaactgtccctggccagtgcacctacttccattaacgggcctaccagcagcgttacagaagagacgacgccattttcactagtcaaggatcctgagcttcttattcagaccgcgtccgaacagtgtggagtttctaaagctgagattgaggatatatacccgtgtactgacctacagcaaattagcgtaccgctggcagatccagctcgtagaaacgtaactttagccacagagattcagttagcgtgggcgattacgacttacacctatacgggatgtaaagatgtgatctttggactagtcagctccggccgtgcagttcccgtaaccgggattgaaagtattttagggccgacgttcgcatgtaccccgcttcgtatatcaatcgatccggatgaagaactaggagaagcattggcggagttacaatatacacttgtggagcagagcaaattcgtccattttggtgcacaccgtatcatgcagctgggacccaacgcggctgctgcgtgcaacttcagaactgttctggtcgtagaggctgaccaaagcgatcgtgccactggatcctggttcacccaacatgaatttctgtccgatgtgacgtcatttagctcccatgtcttaacgttgcgttgcaagctgttccctaagtctgtagaaatcaacgcgatctatgataacgctatggtagatgggagacaaatgcacagagtgcttcttcaattcgaaagaattcttaagatggttagagcccccgagtcagttaatcataaaatcggaggattagacaaattgagcgggagtgactggttggaactacaggcgtggaataccaggctattacccccgcacccaaaaggtatccgtgctcaacaggttattcaagagaaatgtagagcaagaccggacgcgacggcaattcacgcatgggacggggaagtcacttacggagagttagggaaccgtgcggaaaaattagcgggtcttgtgagggcttacatttccaagccaaaccaggtagttgtcttatacttttcaaggagttggttaaccgtcgtggcacagttggcggttcttaaggcaggtgctgcattcatgacactggacataagccaaccagcccagcatctaagggaaatcagtgctacggtcagaccagtcccagtattaaccagcgaagaattatacagggaggccgccgagcttgacatggcagctgtcttaggcattgacaagaacgcgctgagaaatgccactgcggcatctcaaacctgctctactgtggaaagcgacataatgtactctatcgcaacttctggtgccactggcaaaccgaaaatcgtcgtcatcgatcaccaggcgtttatgacaaatagcaggccaatgatcgacagactggggattacggcggaatcaagggtttttcaattttgcggctactctctggccggaggctgtatctgcataccatccctgcatgacagagataatagattggcggcaagtatagtagagctgagagccaactgggcagcaagtccgtcatcagtgctacagctgctggatcccaactcagttccaacgattacgacgttagtgcagggcggagaaagattacaccaagttatcatagacagatgggcttcacacacaaggttattgaacgcttatgggccagcggagtgtagtgtggttgctttcgtgtcagatagcgtgagacctgacacgaaagatccgcaaaacatcggcttagcaactgggggcggggaattgataatcgaagggccgatattgtcacagggatatctgggagacaccgaaaagacaaatgctgcgttcgtgccgctgcccgattggctgagagcttttagaaacggacgtgcccaaggtagaatctacagaacgggcgatattgtcagcttcttgaggaggaaagattcccaatttaaggttagaggccaaaggttagaactgttagacgtagagtaccatgttgaacgttgcttcacaggggcacatcaggttgttgccgacatcgtacccatcccaggcacgcagtcaacagtacttgtagcactgatattagctcaatctgcaatgctagaccaacaagatcttgaaactacatccggcggactggcggttctgcctcaggattctagctttgtcgctgacgccaatgccgcagagcttgccttacgtgataaggtaccatcctatatggtcccagatctgtttgtcccagtctcagacttcccgaggggtgtgtcagggaaagtagataggaaggcaatcaaacaacatctgacttctttattccaacaagactggagaaaatacgtttccactgggaaagttgccccgagcactgccatggagcgtgaactacaaatcatatgggcgagagttttaagagtcgagccggatacattcggtgtacacgatagttttttcagacttggcggagatagtatagtctccatgcaagtagctgccgagtgtcgtgcggctgggatcaacattacggtaaaagacatgtttgaaaaaagagttatacgtaagttagccttgggacgtacgagcactcagcaaaatgggcctatgagctcctccaaggatgatgaaagtggccttatggaagaaaaagcacccgcattctatccgggtggtaagcttgaggtgtatatgaagagagttcagagccagctagggcaggccgtcgagaggatatacccttgtagtcccatccagcaagggatcctgatgtcacacacccgtaaccctgatcattacgatgagataatacaatggagggttatctctaatgcccctattgacacaggaagattacataacgcttggctgcaagtagtgtcacgtcattcaatattgcgtacacttttcttaaatgtgtctaaagatcactttctggaccaagttgtcttaaaacactacagccctgatatatctgtatgcaatggtggcgaggaggcaacctacccgcccttcgaggattttcttccaatgcatcatctgagagttaaccgtttgagcgcagacaatgttactgtctgcctgcgtattcatcatgccctagttgacggtatctcattgcatataattaaacgtgatctagagagtgcttatcagggccgtttggccatgcttggagagcctcctgcctaccacgaatacattagttacttgcagaattctaggctgcagcgttcaagcgaggagtattggaagtcctatctacaaggcgcgacatgttgccttttcccggcacttaaagaccagactgttcaagacaggcagtattttggcgcggtcgacctggagttggaatctactttgaaacttacgcagttctgcaagaaatacaaactagctatgacagttgtattacattctgtgtgggctattattgttcagcgttacactgcgacgcatgaggtctgtttcggttatatgactagcggtcgtcatgctcccgtagctaacgtgcacaacattgttggtccacttttcaacatgcttgtggcacgtgttggtcttgagtatgatcaatctttgttgagcatgatgtacaagtaccaagagtcttttatctcatcactagatcatcaacatcagcccttagttgagacgttgcacagcattggttcatccgcgggcgagttatttaacacgttgatcacagttttcaatgaccagccggaaattcagttagctcaacaacagagcgctttaaccttggtgggagacgccatccaaagtagatcagagtacccaattacgttgaatattctatctagagcggacaagatggaaatgcagttgtcatatcataaaacatggttgtcagccaattttgccgaaaccatcgcaaaaacgtttagatttgtactacaaaatatattggaacagccaaaccaactacttagcgggctacaagtgttagacgaggagcaaatgaataacatttacacacaaaatagatgtgtagccccgccgttaaacgggttcatccatgacgcgattcaccagcaatgcttgagaagtcctgacagccaatcagtatgctcttgggatggtaattttacttatagacaacttgacggtctaagctctgttcttgctgaagaattaatatgtaagggtgcaggtccagaggtcacaataccaatcctactagaaaaaagtcgttggaccccagttgcaatgttagcagtccttacctcaggatctagtttcgtgttaatggactcaagccatcccgttgcaagacttaggacgatttccaatgtaatcaaccctccactaataattataagtgctcagactagatctaaagccacaagtttcagcataaatttgatagagctgggggactggctagccgaaggggtacatatcacagagcagtacacctggcaaaatgtgtttttgaacaaaaacaatgcagcctacctggtattcacttctggctccacagggaaacctaagggagcaatcatagaacacagctcttcatttactggcgcagagtgcctggcagctagattacacattaactccgcgtcaaggattttacaattctctagccatgcctgggacatccccgtaatggagattctgttaactctaagagtgggcggttgcgtgtgtatcccaagtgaagaagagagaacgggcaacttagttcaggctgcaaatcgtatgaaagccaattgggctatgttaacgccgacagttgccagattgtttaagcctcaggatttcgcatacctacaaacgattatcttagcaggcgaggcgcttagttcaactgacatcactacgtggtgcgatagggtgcgtcttattcaggcgtacgggccagcagagtgcactgccattagtactgtgaccgaacccttgacttccagtgataaccccaggtgtgtcgggaggccgtccgggtgcgtcggctgggttgtgagcagaaacaaccatgaacttttagccccaccgggggcgataggggaactagtcctggagggaccgattgttggtagaggttattttcgtgatcctgaacgttcagcgaccgcttttgtgagtccacccgcttggctgttgaacctgagagggcatgaaccaagtattagactttataagactggcgatcttgtgaggcaggatgtctcaagtggattgttgacttttttggggagacaagatgatcaagtgaaggtgcgtggtcaaagagtagagcccggagaggttgaaacgcaagtctcacaggtattccccgagtctcaagtgataatcctagtagttaagcgttctgatacggcgatcttagttgcgcttatccttcgtaaagacgaagcaggaagctgctctacaaagacaggaaacgtaatatcccctccatcacctgcattcgctaagctggtcaggacggcgttcagccgtctgagggagacgatgcctacgtacatgatcccttccttcatgttaccgctggcccagctacctaatgcatccacgggtaaagctgatagaaaacttcttcaggattatgtggcctctctgtcaaacgatgagctagaggaatatattaccgctaatgcttccaggagggtggcaagcacagctgtggaagctcaacttcaggaattggtgggtcaagtcctacataagccgagccacacaatcccattggacgaagatctatttcgtatcggcatggacagtctgacagcaatgactttcgcgacggctgctaggggctgcggctgggaagtatctgtacccataatcttccagcattcaagattatcagatttagcccttcttcttgacagagggcagcacataaagcaggggttattaaaagagactccgattcccaacccattagcgagtttcctgccggagatatgcacgaagtggcacttgcaagaaaaccagataacccatatagttccgacaacgtactatcaacaaatggcgatagcctccgaccattatgttttcgtgggtttacatttctcaaggccaatcgctagcgaagctttaaaaagtgcggcgtccagactggtaaaacaacactctatattaaggacagttttcatccccttccaaaatgcttacgttcaacttaccttgagggacttcgatctaccgagcttagagattcagtcaaacgaggcagagctgtctaccgatgttgaaaacttctgcagagaagcggcgaaaaacgctgctgaattcggggtcccacacacgaaacttgtgcttatactaaataaagagggtggttgtttatctgctttgttaagacttcaaagggctcaagtcgatggggttgcattgatgaggatgattgcggattggaggttcgcgatggaggaggaaccctcattaccggttccaacgctagaatatgccgattttgttctaggtcgtattgcgcaaaacacgcctgaagtcttcgggatgtggagagaccaacagcacatccatactatgactggagcaactgcggattggggcgacgttgtggcgaaaagcacgacatggccgagtgatacagaatttggttcagtccttcattatttgtcagcaccagcgtctcctgcttacgactttcccggggatatatgcggccgtttccagttatacgactataagatggctcatacttgccccctagtcacttgcatcgtattcccagggaggatggatccgagtctaaaggaacttaagatcatagttacctcagcaggatgtggtcaagagatgacggatcaactattaagtatgttccaaatcatgctgcatcaggcaaataaccatagcgataaactaatttctgatcttctgagggtcgagtcc

SEQ ID NO: 81 - DNA, 10665 bp, synthetic construct
  source 1..10665: mol_type = other DNA
atgtcatcctctataacgatacagatagggaactataacagtatggcggtcaatcaggcactgccaataaatcacaacaaatctaatggtgacctgaacgacgacaaagacacgccagatcaggacgtcagttcagacagtggcgtgagtaccgctaataactatcaaccatgtatcctaacgggatttacacctgccccgactcgttcaactcctcctaaagtgcaaagtgcctctgtaaatggaaaagagtcaataagtaagttaaagactttcttagaagagtatgatgtgtcagccgctgccgcgcacaagggagcgtgggcgattgtactaggtacttatcttggtcgttcttatgtgagcctggattatggagttatcaaccaccaagccttagcaatagaggcaacctgtaacgaccgtagaactatcgagataattccttgcactatacgttttaaggaaagtgacaccttattagacattattcgtcaggataccatctgtactcacgccggttcctatcagaagtttgcaatcccgcccgtagagatcgagaaccgttgtaatacctgtgtgatacactggacgggagagtccttccctgagaacatacagagtagcgccctaatgagggttctagtagaagacgagcaattggctaagtatgattgcgtagtgaacttcaccagtgaagtccagtgcataatcacgtatagagataattttatgtctccagaccaagctgatcacttagcagcgacattgttggtagttctgaaccatatcacggacgcgccaagacagtctgtagccgacatcgatattttcagttctctagaccaccagaagctttcccagtggaatcagaaaacgccggtcgcaagcgaagtttgcgttcaccacctaatcgagcaaagtaggcagtcaagacctaacatccaagctgtagtctcttgggacggcagtttaacgtacgacgaaattgatcgtcttagcagcaatctggctcacagacttcgtgaagcgggtgtcagacctggtgtttttgtcgcgatgtgcttagatagatgtaaatggacacctgtcggcatgcttggagtaatgaaagctggtggtgccttctgcgccttcgacccgtattacccagtgagtagacttacatacatgtgccgtaagttgcaaataccaattatccttaccaccgaggacaattttcagcaagcgagtcttctacctggaaccgtatttgtcctgggtgatgatctgtatacagaatccccatatcctggagcacagcaaccacacttcagaagtgaagtctctcccaatgatcctgtctatgtagtatttacgtcaggcagtaccggcaaccccaaagggatattaatggaacatcgttcatttagcagctgcgccctaagttgcatggagccgttgcacataatgccccaagacagaattctacacttcagcagttactccttcgacttgagcatcttcgaaattcttgcgacattggtggcgggggcgaccatcgccattccttctgagaaagcaagaagggagaatctgccgcatactatcacggagctaggtgcgacatgggcattcctgacgcccactgtcgcgcgtatgtacagacctaaacaggtcccctcacttaagacactatgccttggtggtgaggcgatctccgcctctgatgtcgaattatggtccagcaaaaatctgatcaccggttacaacccggctgagtgctgcccactaggtatctctggtcccgccaatcaatacatgccgcgtaatctggggctagccttttcatcccaggccagttggatcgtcgatttgaaggattacaagaagctattacctgtcggggcgatgggggagctggtcatcgaaggccccagcgtagctaggggatacatccacgacccgacatgttctgcttccgacaccccattcctagtgacaccacccccttggctaagccgttttagagccaaccttaatcaaggaactagattataccttacaggagacctagctcgttataatgacgacggaactgtgaatttcatgggtaggaaagaccttcaggtaaagatccatgggcaaagagtcgagcttgcggaaattgagtatcacctggaccagcactttcaattattagcggccaaggtgattgtggaaatcctgcccgtaagcgggcgtacgatcctggccgcgctaatacttcctgaccaaaggctagaaaaagatgaagaacctcaaaactcacttttaatggaacttagcgaagtcaaccaagactttcaggccaagctggccagtgcggcttcaaagctggagttagccctacccaaacacatggtaccaaacatttacttgcttatcaggcacgttccgatgtccgaatcagggaagatagaccgtaggaggctgaagtcccttgtgctgtcactaagccctgaggatttgtatggttgtcgtaagccgacaggccctgataaggaaccggcaagtgatatggagaagttactaaggtccttgtttgcgcaaattctggatgtcccgcatacacgtataaatctggacagcaacttcttccagctaggcggtgacagtgtcttggccattaaactacttgccttggctctggagcaaggcattgtatctatgacatatcaagggatcttcaaccatcccactctgagggacctttccctagccagcacccccacgagttcaagcgttcccacgccacttgtcgcagaggagacaaccccattctccttggtaaaggacccagagcttcttattcaaattgccagcgaacagtgtgttgtatctaaagccgatattgaggacatttacccgtgcataccattacaacaaagtactatggctagtacagctcacaatagtagcgcgtatgttgcggttttaagcttcacactgagcggcgggattgacagagaccgtcttaaacgtgcctggcatatcgctctgaacggccatgctatgttacgtacgaggctaattcaggctgatacaggcgattgctaccaagtggtgataaggggggccagttgctggatcgagaatcgtgaattgtcagatggggacacaactaatttatgccgtgcttccttcggcctgggactacctctgattcaagcacacttagctaaggacaggctgtacgtggcgatgcatcatgccctatatgatggttggtcccttcccgtattgatcgatgaattagatcttgcttatcgtgagctatccgtgcgtagacttcctagcctgaaacactacgtaaaatacatgatggactccgcagcctcagccgctctgttctggagggccgaacttgaagatgctgaaccggttcacttcccggcacccagtaggttagactacaagcctcacccctgtgcgtccttaactgtgagtgtacccttagtagattcccccaggcgtaatgtcactttagccactgagattcagctagcttgggctgtgaccacatatgcgtacacaggctgccgtgacgtaatctttggactaatgtctagcgggagagcagcacctgttccggggattgaatcagtacttgggcccacgttcgcatgtacgccgttgcgtatatcaattgacccggatgaagacctaggagaagcgttggaggaactgcagtacacgttagtagaacaatctaagtttgtccactttggagcacacaagattatgcaattaggacagaacgcggccgcggcatgtaatttccaaacagtcttagcggttgaggcagatagaccggatagtgtgacaaggtcttggtttactcagtacaaattcctaagcgatgtcacgtccttcagctcccacgctctaaccttgaggtgcaaacttttcccaaaaggtgtcgagataaatgcgatttacgataacgctatagtagacgatagacagatgaggagaattctgtctcagttcgagcacgtacttactcaagtccacgcaacagagtctgtaaactgcaagatcggagggctggttaaactatcaggctccgattggctggaagtccaagcttggaataacaccttgctgcctccgcatcctagaggaatccgtgcccagcagatgatacaagagcagtgcagggcacgtccccatgcagctgctattcatgcgtgggacggtgaggtgacatacggggagcttgaaaaccgtgccgaaaaattagctgacttagtacgtacctatatatctaaaccaggccaatttgttgttttgtactttagtaagtcatggctaaccgttgttgcacaattagcggtgctgaaggcgggagcggcatttatgaccctagacatttcacaaccagcacagcaccttcgtgaaatatccgtggcggtgcgtcccgtactggttctgacatcaggcgagctttactcagaggccgcagaattggagatggctacagttctgggaatagataaagacgcattaagtaacggtcccgtcgcaagcctaacctgctctaccgccgagtctgatattatgtacggcattgcaacgagtggaaccaccggaaaacctaaaatcgtcttagtggatcatcaagctttcatgaccaactcaagacccatgattacaagattagggataacggcggaaagtagagtcttccagttttctggttattcatttgatctgatgattgttgagcattttcttacacttcttgccggtggctgcatttgtataccatccttacatgaccgtgacaatagactggccgcgtctatcatagaattacgtgcgaattgggccgcctcaccatcttccgtactacagctacttgatcccactagcgtacccacgataacaacacttatgcaaggcggcgagagactgcatcaagtcatcatcgaccgttgggctccccatgttcgtttattaaacgcctacggccctgccgagtgctccgttgtcgcgtttgtttccgatcccgtccgtcccgatacaaaagacccccaaaacttaggatttgctaccggaggtgtcgggtgggttgtgaatgccgatcacccggatagtttcccggtacctatcggcgctgagggggaactaatcattgaagggcctatactaagccagggctacctggacgatgcagagaagacttctgccgcattcgtgccacttcccgattggctaaaagcctttcgtgatggtagagcccaaggtaggatatacaggactggtgacatcgtgagacaaaacagtgacgggagcatttcatttttaaggaggaaagattcccaggtaaaagtcagaggacagagactggaattgctagatgtggaacaccacgttgaaagatgcttcgcaggtgcataccaggtcgttgccgatattgtacagattcccgggacccagagcacaatgctggtcgccctaattttaactcaaagtgcaattttggaccagcaagatctggaaaaatcctcaggaggactggcgattcttccccacgatccctctttcatagctgatgcaaacgcggcagaacttgcccttagagacagagttcctgggtacatggtaccggatctattcgtacccgtgagtgaattccctcgtgagttgtcagggaaagtagggaggaaggctataaagcagcacttagcttccttattccagcaagactggaggaagtatgtatcaactgggaaggtggcaccgtctacagcgatggaaagagagctgcagtccatatgggctcacgtcctacatattgagcccgattccttcggggttcatgattccttttttagacttggtggggacagtatatcatctatgagagtagcgaccgagtgcagcgcggtgggaataaacattaccgtgaaggacatgttcgaaaagagaacgattcgtaagttagcacttggaagaacgaccacgaggcagaatggatcctcaagtagagacgacaactcagggatgatggaagagaaggctccaactttttacccggatggcaaattagacgtttacatggagcgtgtgcaatcccaattaggtcaggctgtagaaggcatatacccgtgttccccggtccagcaggggattttgatgtcccacatgaggaaccccgatcattatgatgaaattgtgaggtggcgtgtcgcgagcaacgcacctatcgatactagcagactacaaaatgcgtggttgcaagtggtatctagacatagcatcttgagaacgctattcctgaatgtaagcgatgatcattttctggaccaggtagtccttaaacactattcacctgacattagtgtatgtaacggtggagaggagatgaaatttccaccatttaaggactttcttccgttgcaccatcttaggataaaccacctatcagctgacaacgtaagtgtttgcctacatattcaccacgcacttgtagacggcactagtcttcagataataaaaagggatctagagttcggttatcagggaaggttggccgtattagatgagccaccagcttatcatgagtatatatcttacttgaggaaaatgtgtctaaaaaaatctcctgccgagtactggaagagctatctgcagggtgcaacttgctgcctattccccgcattgaaagacagagcggcgcaggacaggcagtatttcggagcagtcgatttagaattggggcctacagtcaaactgacgcaattctgcgaaaaatacaagctggcgatgacagtagttctgcatattgtctgggcgatcatcgttcagaggtatacagccacgcacgaggtatgctttgggtacatgacctcaggaaggcacatatccgtcgccaacgttcataatattgttgggcccttgtttaatgtgttagtgagtcgtgttgggttggcatacgaacaatcattgctaagcattatgtaccaataccaggagagttttgtctcaagtcttgaccaccaacaccagcctttagcagagaccctgcactcaatcggctccagcgcgggagacttgttcaatactttgatgactgtctttaatgatcagcctgaaatccagttggaccagcagcaaagtggcctttctctaattggcgatgctatacagtctagaagtgaatacccaataacgttgaatatctttaatagagcagacaaaatgcaaatgcaactatcctatcacacctcttggttatccgctaatttcgcggagactatagccaatgctttccgtttcgttcttcagaaaatcttagaacaacctaaccaattactaataggattgcgtatcttggacgagaagcagatggataactcttatgcccaaaataggtgtgtggcaccccctttaaatgattttattcacgatgaaatacatcagcaatgccttagattcccagactcagagtctgtttgttcatgggacggtaacctgacgtacaggcagcttgatggcctgtcctccgtccttgcggaggaactaatccgtaagggggccggaccggaagtaacaattccaattttgttggagaaaagtcgttggactcccgttgctatgctggccgtcctgaaatctggcagtagttttgcgttaatggattcctcccatccggtcgcaaggctgaggacaatctttacaacgatcaatcctccggtaattatcgtgtccgctcaaactaggtcaaaagcgacttcattctcaaaaaatatgatagagcttggagattggctgactgagggggtacacataaccgaacagcacaactggcaaaatgtatttcttaaaaaggacaatgcagcctacctagtgtttacctctgggagcaccggggagccgaagggtgcgataatcgaacatagtagcctgtcccaagcggctgaatgtttagccgcaaggagtcatatcaatagcgcatctcgtgtacttcagttctcctctcatgcttgggacgtgtcagtcatggaggttttgctgacactacgtgtgggcggttgtgtgtgtattcctagcgaggaggaaagaacgggtaaccttgtacagactgcaaatcgtatgaaagtcaactgggccattttggcgcccactgtggctcgtctatttcgtccgcaggacttcacacatttgcgtaccttagtcttagccggagaagccctgtcatcaaccgatataaccgcgtggtgcgagcgtgttagactgattcaagcgtatggccctgcggagtgcgctgttatttccataacgactgaaccgttgactccgtcagacaacccaagatgcataggcaggccaagtggatgtgtcgcttgggttgtaaatagagactcacatgatctattggcaccccctggagctatcggggaacttgtcctagaaggccccatagtgggacgtggatacttcagagaccctgaaagaagcgctgctgcgtttgttaacccgcccgcctggctgttgaacctaagaggccacgaaagctctatccgtctttacaagacgggggatttggttcgtcaagatgtgagctctgggctgttgaccttcatcggccgtaaagacgagcaagtaaagatcagaggacagagagtcgaaccaggggaagtagagacacaggtcagtcaggtgttccctgggagccaagttctagtgttggttgtcaggagaagcggcaccactatcttggttgccctaatccttcaacgtaatgagaattcttcctgcccgacgaaaacaggcaatgtgttgctacctccatcaagtgcttttgccgaaagcgcaagggcagcgttttctaggttacgtgatactatgcctacttacatgcttcctagttttattttgccgttggcccatctacctaatgcgagtacagggaaggccgacaggaaactgttgcaggattgtgtagcgagcttgagcgacgacgagcttgaagagtacacagccgcaaatacgagtagaagaatcgccagtaccgcggccgaggcacagcttcaagagcttgtgggtcaagtattgcataagccacctcatacaattcccttagatgaggatctgtttaggtttggaatggattcattgacagctatgactttcgccacagctgcaaggaggtatggctgggaagttagtgtccctataatttttcaacattccaggctgtctgaccttgctctgcatgtgaatcgtggaggtcaacacaacaagcacggccctctacaaagagctccaataagtaacccgctggtttcctcactacctgagatttgcataaagtggcatcttcaagaggaccaaattacccacatcgtcccgacgacgtattaccagcaaatggcactggccgctgaccatgttgccttcattggactgcattttagcaggcccattgcatccgaagcattaaagtcagcggcctcaaggctggttaaacaacattctatcttccgtacagtattcgtgccttttcaagacgcgttcgttcagcttacccttcgtgattttgaccttccctctcaagagattcagtctaatgaggcggaattgagccgtgacatcgagttattctgcagggaagcggcgaaaaaagcagtcggctttggagtgccaagcacgaaattggtgctggtactaaataaagagggggggtgccttagtgcgttgctgagacttcaaagagctcagttcgacggcatagcagtcatgagaactatggcggactggcgttccgcaatggaagaagaaccatcactatccgcccccactatagactatgccgactttgtattagagagaatcgcgcagaataccccggaggtgttttccatgtggagagatttgctggaaggctcatccatgacgtacccttttcctcagcatgaatatattaggatgactgacagaagtcgtacagaaaggcttgtcgaaagctcttgcgatataccgttgccggaacctgcgcgtggttttactatggcaaccgtcgcgaaagccgcgtgggccatctgtttagctcgtcaggcacaaacacaagatgtgctatttctacaattggtcagaaatcgtcatttagtgttggacgggattgacaagatggttgggtgttgcataaactacattcccgtaagagtaccgctgcaacccgattggacagcatctgacctacttcactggatacaacagcagcatctgcacactatgacgggtgacacggtcgattgggatgacatggtcgcaaagtgcacaacgtgggcttccgatacagaattcggtagtgtccttcattacctatccgctccggcgtcccccgcctatgactttccgggagaaatttgtggaaggtttcagttgtacgacgataaaatgcttcacacatgtccatccgttacctgcattgtcaacccaggacgtacagatccatcaccacgtgaactaaaaataatcgtcacaagtacggtgtgtagtcaagagatcgctgaccgtcttcttagcatgtttcagacgatgctgcaccttataaataaccacccgaatgctttagtgttggaccttgtgagagcaagggacccggccatcggtggagcaaagctatgtgaatta

SEQ ID NO: 82 - DNA, 3915 bp, synthetic construct
  source 1..3915: mol_type = other DNA
atggcagccgccaccttagaggactggaaagcctacctggaaggttacagcccaggaaattttcccaaactgagcgggataggtgatgtcgacttcgccacatcaacaagcgtgacagctacgctaagggacccatactgcaaactgcaatccttctctaacgaacatgcggtcgacacggccacagtcttctgtgtgggttggggggtggttttgcaggcttacaccggtcaggacttaatttgttttggaatctctaagccgtcaatttccccttgtaggatagatttcacgtcaaaagcagtgataagagatatattagcgacttttcaaaatggatttgacaaagaaccacttcatttggaaattcccatgagtcgttttctgcaaaacgataagcacttcgtcacgactttttttaacacgtgcatcttatatccaaaccgtgaagttcgtgtccagaatggggtaagcgacacatccgcggactgcaacagcctaacggaattagatgttgtgattaggtatgatgatgaagagggcattgtccgtattcacatgacgtataagacctctttactgcagaaggcgcaagctttaacggttattaacgccctggagagagctgtttccgaaataatcaacggtaaggatgtggtggaacagctttgccttttaagcacggatgacagacatcatatttttagtcaaaaggttagactgcccgactcttccgatgaccaaattgacaaacttattagagacagatgtaacgaaaggccgagcgcgttagcggtttgcgcatgggacggtaattttagttacatggaactaattgaacttagtaccgccctagcccaacatttggctgtccagggcaccggaccagagatttttgttcccgttttattagagaaatccaggtgggcaatcgtagccatgctaggtgtcatgaaggctggtggggctttcgtactactagaccctagccacccggaagagaggctaagggatatctgtaaacaattcagcacaaagttgatcatttctagtagtcaacatgctcagttggcagcctcacttgggagacaggtagtgatggtacaagatggggaagaaatagggtggaatagaggcggttatcactttgttaaatcacccgtgaccccgaatagcgccctttacgcagtttttacatccggttcaacaggtacaccgaaaggtgtcgtaaacaaccactccagttttctagctgccatgccaacctacctagacagcgtcatgcttaacaaagacagcagagtttttcaatttgcgtcatacgcattcgatgttaccatttttgacacccttatgaccttggtcgcaggagggtgtctatgtgtgcctagcaataacgaccgttccagtgacttgacaaatgcagtacaacgttttgcggccactcacctgagtctaacaccgacggtagccagggtgttagacccacaagactttcctacgctaaccacaatcgtacttggaggcgagtgtcttatacccagcgaaatttttagatgggcagaccacgtgcgtgtcgttcatttatatggcgcaaccgagtgcgctataatgtctgttcagtgcacatcaggaactccgcctaatagtagaaccataaactatgacaccgggtgtgcctgttgggttgtcaacccgagaaaccccgagaagcttcaaccggtaggagccataggcgagctaatcgtagagggaccggtggtgagcagaggctacctgtcagacccaacgcgtacctcagagactttcatcgaagctcctacctggcttagagaattaaggggaaagagatccactgtgtataaaagctccgaactggtgagaattgccggcgacgggacaatccaaattatcggaagaaagaatatgcaaatcaaattaaggggtcaaagaattgagcttggtgaggtcgaacaccatctaaaactagcaattccctatgttgccgattgtattgctgagttagttacagtaccggacagttcaagaccacccatgctgatgggttttatcagatcagcattcgatgtaggcgttcaaagcattgcgacgggccacaatgcgacagcttccaacgtcttcgccgaaccaacaagtgcgttcagaagccaggttcctagcatcttaagttccgtaagtaagtctttaccctcttatatggttccgagtactataattccccttcgtaccttacctttgacttccaccgataaagtgaatagaaaactacttagggagctggccggcggtctatctcgtgaggagttcggaaagtatctgccccatgctcgtatatatagggcaccaacaaacgatatggaagaggcgttacaaaagtactttgcgagcgccatgaatatgtctgtagagcaagtaggtgtggacgacaatttcttttccttaggaggcgattctttgaccgcgatgaagctagtcgctatggcccgtaaagcccgtttgagtctaacggtccaaaatgtctttgatcacccccagctgagcgagctggcaaggaatgcgcagggcgttagtgatgagaatcgtgatcagccccagccatatgcgcttataggagataacaagagagatgttattcgtgacgctgcgcgtcaatgccaacttcccagcagggctattgaggacatatatccctgtactcaagtgcaaatcggtttgatgaccgaaacaatgcgtaaccctggtttatttatagcaaatatagcacttccgctaaggggagacattgatattgaccgtctgaggcaggcttggaccgcagttgcaaaggctaaccctatactaagaaccaggatgattttatcctcttctcatggaatgcttcaagccgtggttagggaggacattaggtggattgtgtctgatgttccgtccccacaggacatggttggatttggaaagcccctagtccagctggtacttagtctgatcccggaaggtgtccccggccagaataaactgctgattacaattcaccacgcaatctatgacggttacacccttcctttacttttcgaacaagttgaaactgcatattcaggtggcgtcttggcaagccgtcccgtgtccccatttatcagatatctggcccaagtaccaaatggaactcagtactggcctgcgcttttcgaaggtttagaaacgccgcccttccctccattgccgtacgagtctcatctgccatcacctgacagccaggaaacgctaaatgtgtcagtctccagcccgtctgcaaaacagtacactcctaatacgtacctacatctagcctgggcaataactcaagcccatcagcaatcattgagggatgtattctacggggccgttgtgagtggacgtggcgcaccggtggacgacattgaattgatgacgatccctacggtcgctacaataccttgccgtgtgtctcttgacgcaggtagcaccgtgcaacagacgcttcgtcgtatacaggaagtggcaactaaagggataccctacgagcagctaggcctaatagagattcgtagattaggtaagtacactgcccatggttgttctttccagaccgtgctggttttacagcccgccgtccaaagaggggtctctgatttatttgaaatacccgaagccgatgtaaactatagacaggatgccacatacgcggtgaatcttttctgtgaactgtcacaacagcaactgatagtaactgctttctatgacgagagaatcgttcccagatcaaggatgcaagaaatccttgagactttaggtaggacccttaaagccattcacaccgctcccacatccgtgataggagatatcctggggactctggtgacatctcgtggatcaacg

SEQ ID NO: 83 - DNA, 10758 bp, synthetic construct
  source 1..10758: mol_type = other DNA
atggcagccagtatacccattccagaaaaactgcaagatttaactgccgtcaaatccccaagtgcctttggaaatagcatagaatccatcaacggagacaaaaacaaatctgagagacatacggcaagttcaagtgctgttagcacctctgaaattggccatccatgcctgcttaccaacttcaaactagccgtcgcatgttctggaccggcaatcacgaaggtcgcaacggttaacgacaaggactccggaagtaagctgaagaatgtgataaatgggaaggacatcagcgcgagtgaagtgtttaaaggggcctggtcagtagtactgggtacctatcttgcgaaaagtcatgttagcctagattacggcgtcatgaagcctaaaggcctaggtcctgaaactagttgtaacgcgagggtcccttcagaaaacagtgaaatgagcacttctagcttcctgctgagggctaacgacacactattggatattatacgtcaaaactccatgtgtgctcataccgaattgagacagaagagtagtctagatgatgtcgagtcacctaagcgttgcaatacgtgtgtgatatattggccagagattagctgcagtgaacaacttcaaatagacgcttggatgacgatcctggaggagaacgaccagttgacgcagtacgactgtatgatacatttcgcttccgacatgcgttgcatgttatcctaccgtgaccagttcatgagcgaaaaccaggcgaggcatttggctgctacgatgagagttgtgttatcctccattgcttctgcccctcaacaaagcctagcagatgtggatgtttgtagtagcctggactaccaaactctatcaaggtggaaccttaaagctcctatagtctccgaggtctgcgtccacgaccttatcgagaaatcttgttgcagtcgtcccaatagccaagcggttgtgagttgggacggttgtcttacatacaacgaaatggacaggctgagctcccatttagcgcaaagattaagagatgcaggagtggagccgggcgtctttgttgcgctttgtctagataggtgtaagtgggcagtgattggtattgtggccgtaatgaaggcggggggggctttctgcgcgcttgaccctagttatccagtgtccaggttaaaggaaatgtgccgtgaccttgggataactattgttttgactgttaagtccaacattcagcacgcaagtccccttgcatctaaggttttcgctctagacgacgacgtatacttcgaatcagctttatcatccgcgcacgagtcagcctcctgggtctcagtaagtcctcatgatcctgtctatgcggtgttcacttccggatccacaggcaagcccaaaggtatcattatggaacacgcgagtttctcagcctgtgcgttaagcagcgtcaaaccccttcagatcgcagatcaggacagggtcttacatttcgcatcttacgctttcgatgcatctgtaatagagatacttgctcccttaattgcaggcgcgacagtcgcgatcccttccgagagggccagactagaagacttaccacgtgcgatgactgacttgaaagcgacatgggctttcttgacgcctactgtggccagactatatagaccagagcaaatgcccacattaaagacactttgtttgggcggagaagcagtgaacgcatccgatacgagaagctggagttccaaaaaccttatcagcggttacaacccagccgaatgttgccccctaggcatatcaggtccactgaatgaccgtatgccaaggagtttagggagtacctttgcgtcacaaaccgcgtggatcgtggacccgaaggaccatgagaaactgttgcccgccggcgcaataggggagctggctatcgaggggcctgtcgtggccaggggctatatacatgatgtgacttgttctgatccaagcacaccttttgtggttaaattgccgccctggttaagaaggttccgtgctactgcaaaccgtggaaacagaatctacttgactggggacttagcccgtctggattgcgatgatggttctgtccactacctgggtcgtaaagacgaccaagttaaaatccacggacaaagggttgagttagctgagatagaacatcacctagaacaacactttgtgtctttagctacgaaggcggtggttatgctactacgtcctattagcggaaggaccgtcttagccgccttgataatgcctcaccaaaggcttcaacacggcaataaatccttagaaagtctattgatggagcccggagacgtgtcacaagatttccgtgccaacttggcatctgcggcatcaaaactaaggctggcactgccatctcatatggtgccaagcgtgtatttaccgataagacacttttcaacaactaagtcaggtaagattgacagaggccatctacagagtctgctgttatccctaagtccagagaatctttacggctgcgaggaaaccacccatcgtggtgaagagccaaagagtgatcgtgaaaaattattgcaagcgctatttgctcaatccttagacttgccctgcactaggatagacttagattccaacttctttcagttaggaggggatagcttaagtgcgatgcgtcttctagccttagccttagaagaaggtatatcttccatcgcgtaccaagatatctttagtcaccctactttgagagaaattgtgatagtgagcacatcagcgaccagccgtgagcccctgtatagtgagactgtggagaccccgccgttctctttgatcaaggatcctgagatgttgatacaaatagcctctgagcagtgtgggtcaggcgttggaaaagctgatatcgaggacatatatccttgtactcacctgcagcagtccttaatggcatccaccgctcacaacccaaatgcatatgtagccatattggcgttcaagctaaaaagtggagtagataggactcgtttagagagagcgtggcacatagcttgtagtgggcacgcaatcctaaggacaagacttgtgcagactgacacaggggattgttaccaggtggttgttaaaaaacctcctcattggactgagacgaatgaggttagcgatgatggctctactaatagcctgttgcgtacaagcttcggtctgggccgtccactaatacagctgcatctaactactgaccaactgtttgtcgctatgcatcacgcgctatacgacggctggtcactgccaatgctaattggcgaattagacttagcatacagagaactgtctgtgcgtaggctgccgtgtcttaaaaattatgtcaagtatgcaatggactctgcagacgctgcagccagcttttggcaggctgaactgcaggatgccgatcctgtacactttcctgcaccgtcatctttagactataagcctcaaccgtgtgccgccatgacgatctcagttcctttagtcaacagtccaagaagaaacgtgacccttgccaccgagattcaattcgcatgggctatgactgtctacacatacacaggctgtaaagacgtcatctttggtctgattagtagcgggcgtgcagcgcccgtagctcagatcgaatcaatcttaggcccgacatttgcgtgtacacccttgagagtatccatcgatccgcagggcaaattgggtgaagccttagatgatcttcagtatacgatagtcgagcaatccatgttcgtacactttggcgcacaagcgatacgtcaactgggacctaatgctgctgctgcgtgcaatttccagaccgttttagctgtagaggctgacggtcctgagacgggtgaggaagaaggcagctggttcaccagatacgacttcctttcagatgtagcatccttttctagttatgcgctaactcttagatgtaaattgagtaccagaggtgtcgaaatcaatgcggtatatgataagctaatggtcgatgaacgtcagatggggagaatcctagcacaattcgagcatattttaacacaaattcattccaacgagactgtacatgacgacattggtggcttagacaagctttctgtatcagattggaggcaacttcaagcctggaattcaaatcttccacctgcgcaccctaaggggcttggcgcgcatcaagcgattcaagcgaaatgccaggcccaacctgatgcaaccgctatagacgcctgggatgggtgtgtaacatatggagagcttgagagaagagccgaaaaacttgctggactagtgaggagccatgtgtcaaagcctgatcaagtcgtcgtgctgtacttctctaagtcatggcttaccgttgtggctcaattagcggtgttgaaggcgggggcagcgtttattaccttagaaatatctcagccggtccattatttgcaaagggtcatcagcgcacttggtcctgtgttggtcttgacgtcagaagatttattttcagctgctgaagaccttcaggataacgcggtccccgtgatggctgtagataaggatgacctaagtgacgctacagcaagaacaagtcaggccagtagcagcgcgtgcactgttgagtgtgacttgatgtatattatagctacttctggaacgaccggtatgccgaagatagttatgactgaccaccaggcgtttatgaccaacgcatctcctttaatgaatggtttaggcattacgagcgatagcagagtcttccaattctgcggttattcctttgatcttctaattgtcgaacatttcctgacacttctggcggggggctgtatttgcattccttctttgcacaacaggaataatcgtttcgcggcgtctatcgtcgaattagaagcgaactgggtaggatctccgtcatcagtcttgcagctattagatccacagacggttccgacggtgaaaacgataatgcaggcaggcgaaaggcttcaacagggccttgtagaccgttgggcgtcacatgtgagactaataaacgcatatggccctgcggaatgtagcgtgggcgcacttgctagggacactgttagacctgacaccgacgacgttcaaaatctgggcttcgctacggggtcagtttgttggatagtgaacgcggaaaccagcgagaaattgcttcccgtgccaatcggggctgaaggggagttaataattgagggacacactcttagtaggggatatttaggtgacgctgataagacgaatgcgagtttcttgaggctaccgaactggctgcgtgactttagggctgaccgtggtcaatcacagggtcatagagtatacttgaccggagacatagtacgtcagaacagtgacggtagcatgtcttttgtccgtaggaaggacgcgcaggtaaaaattagaggccagcgtgtagagctaacggatgttgaacaccaagtcgaaaggtgcttcattggcgcacatcaagttgtgacagacattgtgcaaatcccgaattcacaatcttcaatcttggttgccttggtgctgactaaggatgctatgacaaaccataagcagcaggagtcattactggatcaaaaatctgcgggaggcttatctattctggctccaacaagcagctttaccgcaaatgcaaacgccgccgagaccgccttacaggataggatgccggcgtacatggtcccggacctttttgtccctgtgtccgatcttccgagggaggcctcaggtaaaataggaaggaaggcaattaagcaatacctggcctctttgactcagcaggactggtcccgttactcttccaccagaaaagtgccccccagcaacgctacggagcacgaaatatccgccatctgggctagggtcctacaaattgagcctcacacttttggcgtccacgatagctttttcagattagggggcgactcaatatcaggaatgcaagtagctgccgcgtgcggagcagctggtatatctgtcacagtgaaggatatgtttgaatataggacaatcaggaaactggcattggctagaggtgagacgcaacaactgaccgtgggcactacgagcacggttagcaacgccagtggcattcgtcagaaaaaagccttgcatccattttacccagaagggagattagaagtctatatggagaggatgcagtctagacttggacaggccatagaaaggatatatccatgtagtcctatccagcagggcatacttatgtcccacgcgcgtaatcctcaccactacgacgaagtgattcaatggaaggtagcaggtgacgtctcttgcgacatcagtcgtatgcagagggcgtggagagaggtggtatcccgtcatgggatccttcgtacccttttcctacaagtgtctgaggattcatttcttgatcaagtggtgcttaaaaattattcacccgatatctcagtctgtacaaatgaagaagacgtagaaccttataggccattcgaagactccgtgccgatgcatcatctacttgtatttcaaaggagtgcagacgatgtaactgtgtacctgcgtatccatcacgctttggtggacggcctaagtcttcatatcatcaggcgtgatttggagcttgcataccagggtagactggatgagcttgctcagccgccgggctaccacgaatatattagttatttgcaggagaagagatccagaaaaagcttgcaggagtattggtcttcctatctgcaaggagccacgggcagccttttccctgcggtacaggacgagccggcctctgacgggcaatactttggagcggtggagattgaacttgggagcattgcgaaactgacacaattttgtgaggaacacaaattaggagttactgtagtgcttcatgtcgtatgggctatcattgtgcaaaggtacgccgcgacagacgaggtatgctttggctatatgacatctggaagacatgtgcctgtaaccaatgttgagaacgtagtcgggccattatttaatatgctgatcggacgtgtgaaactggcctaccacttatccgtcctgtccacgatgtatgcttaccaagagaactttatcaactccttggaccaccagcatcagagccttgtcgagacactacactccatcggtagttctgccggtgacctgtttaatacgctgatcaccgtggtcaacgaccagccggaggaccatgtgagtcagtccgctttacgtcttgtgggcgattccgtgcagtccaggagtgagtatcctataacgctaaacatacttaaccatgcagacaagatcaaaatgcagttaagttaccacacctcactattgtcaggggtttctgcaaataccatagcaaaagcattcagatttgttttgcaaaggaccttggaacagcctcacgagttgttaagggcgttgcccgttctggacgaagaccaaatgaataacgagtttcaaaaaaacaggagtgtgccgccccaagtcgaggaactgatacacgacacaattcatcagcaatgtatcaggtgccctgattctcccagtgtgtgcgcatgggacgggaactttacctatcgtcaattggatgatctaagcagtgcactttcagaagagatcgtgagaaagggtgccggaccggaggtgacaatacccattgtgcttgagaagactcgttggactcctgttgccatgttagcagtcctgaagtcagggtcatcatttgtcttaatggactcaacccatccagccgcgcgtctaggtgcgataattcaagatgtggggcatccagttattatagtgtcagcgcaaactaggtcaaaggtagcaacattctccaccgatgtggttgaagtaggtgactggctagcacgtgagatactagtcgccaaacaacagattacaagacaaaatggcttattacaggcaaccaatgccgcgtacctagtgtttacctcagggagtaccgggaagcctaaaggtgcgattgtggaacacgcgtcattatctaccgctgcgaaatacatggcgagccgtttacatatagattccgcatcccgtgtgttacagtttagcagccacgcttgggatataccagtcaccgaggtattagtcacacttagaatgggcgggtgcgtatgtgtgcccagtgaggaggagcgtactggtaacctggcgaaagcatcagaacgtatgaaggtaaactgggcgctttggactccgacggtagcccgtttatttaagcccgaagagttcccacatctagaaaccttggtcttcgctggagaggcgctgtccgctgctgatctagagacgtggtgtgacagagtaagacttatacaaggatacgggcctgctgaatgttctttaattagcacagtgacggatccacttacgagatctgataacccacgttgcattggcctacctagcggttgtgtggcttgggtagtgaaccgtgatacgcacgaattacttgccccccccggcgcgatcggagaattagtcctggagggtcctatcgtagggaggggataccttggcgacccagagagagctgcgtctgcatttataagtccacctgcgtggctgatgaaacttcgtggcagtggtagcagcattagactttataaaaccggcgatctggtaaggcagcacgtatcctccggtttgttaacgttcgtggggcgtaacgatgatcaggtgaaagttcgtggtcaaagagtagaaccaggggaggttgagggacaagtcgcacaagtctttcctggaagtcaggtgatcgttttagtggtcaaaagatccgctggggccgtacttgcagcactggttctgcaaaatggggaagaccgttcatccgcgggggagaccgccaatttatttcctccgccttcccttgccttcgccgcgctagcaaaggccgccttttccaaactaagggaaactatgcccacatacatgattcctagcataatattacctctaagctatttacccaaagcagccacaggtaaagcagaccgtaatctgttaagggatcgtgtggcttccttatctgacgaggaaatagaggcttatgttgcagcaagcgtatctcacaggccagccagtaccgccatggaagctgaattacagcaattagtaggccaagtcttgcaacgtcctttgcattccatcagcttagatgaagatttatttcgtcttggcatggatagcctgactgccatgactattgctagcgccgcccgtcgtaggggatgggaggttagcgtcccaatcatattccagcacagtagattatccgacctggcacgtatagtcgaacaaggccagcatggaacgtcaagtaggtcccagctggaagaagccagggcgattttgaataagcgtctggtatctctgctaccggagatatgcactaaatgggatttaagagaagaccagattacgcatattgcgccaacgacatattatcagcacatggctctagcaagcgatcatgaggcattttttggtctatacttcagtaagcctatggcatcagaagcgcttaaggccgcggcgagcagagtggtcaagttgcactctattttgaggaccgcttttgtcccgctggaagacacgtatgttcagcttacgttgtgcgattttgaccttccttcccaagagatacaaacgaatcaagctgaggtatccgcggcgatggaattgttttgcagggacgcggccgacaaaactgccggcttcggcgtgcctgtaactaaattgatactgatgttggacaggcagggagactgtctgagtctgttactgcgtctgcaaagggcccagttcgatggagtcagtgtgatgcgtattatggccgactggaggtcagccctggagcatgcttcttgtagctgggagccggctcccagcctagattacgcagatttcgccttggggagagtagctcagaacaccccagacgtattcggcatgtggagagatgtactacagggatcttccatgacctatctgatacctcaagagaagtatataagtatgaccgacagagcccacgctgaacgtttggtcacgagttcatgtgatatcccgcttcctgaacctgcaccgggttacacgatggcgacagtggccaaagccgcttgggccatatgcttagctcgtgagacggaatctgaagatttactgttcctgcaattagtacgtaatcgtcatctggccttggatggcattgacaagatggtcggctgctctttgaactatgttcccgtcagggtgccccttcgtagagattggaaaatcagcgatttactgcattggcttcatcagcaacacataaggacgatggccggtgacacggcggattggccagacgttgttgcaaagtctactacatggtcaagtgatacggaattcgggagcgtgattcactacctgtctgcacccgccgcaccagtatatcacttccctggtgatacggttgcccagttccaactgtacgatgagaagatgacacatacgtgcccgctggtgacctgtgtggagttcccagggccagccgaggacagcgggcgtcaaatgaagattttggttactagtgctgtgggggggcaggatatggtagatcgtctgctagccgttttcagaagccttttgtgtgaagcgaatgcgcagttagatcagcctcttagcaacatactgcaggggttgcgtgacggggatgatgcaacggggaaagctcgt

SEQ ID NO: 84 - DNA, 3909 bp, synthetic construct
  source 1..3909: mol_type = other DNA
atggctacaccagagaagtggagaaaatatctagaggactatataccgtgttcctttcccacctttctagataacgaaggtgccgactcaaaaagttttgccagtgtcctggtgagattggaacacccttacggccgtttgatgtccttcagtaataattgcggggtcgacgttgccgtggtgttgaatgtggcctggggaattgtactgcaggcatatactggacaggacgcgacatgcttcgcggttattgccgaatcaaaccttaatatccgtccatgtaggattaggttcacgtctgacaaggtcgtaagcgatatcttatccgcttgccagagtccatgcggggaaaaaactggagatcacgatattccggcttcacatctgtcacaagacgggggatttttggcatcagaattctttaatacttgcatctgggggccgatgcaaggttctcagatgcccagtgagacacaggcggcggacatgaatcgtaacgccttaaatctttttgacttagtaactagggtagaggtcgacaaatcaataacaagaattacactaacctataaaagaggcttaatgcgtgagcaccaggcgcttgccgtggcgaaagcaatggaaagggcaattagcgagattatatccggaaaggaaaggctagatcagttctgtttgttgacgagcgaagaccgtcgtcagatgtccctatggaatatgaatttaagcgataatagtgatgcacgtattgaaacgcttatacatgaatggtgtcgttggacaccgtctgctgtagctgtttgtggatgggatggggacttttcttataaagaactaaacgagctttcaacgggggtgaaacatgatctaaggcatcttggaataggaccagaggtatttgtaccaattctattcgagaaatccagatgggctgtaatagctatgcttggcgttatgaaggctggcggtgccttcatcttgttggaccccgcgcacccccctaaaaggctaaggagcatatgcgacaaagtatccgcgcgtcttgtcgtgagttctgtacagcaggcggatctagcggctggactggccggacacgtcgttattgttggcggtgaagttgcgactgccggaatggctcagcacgtcggggagcacgacgactccatggattgtattgccgcgccgcacaacgccttatatgccgtgttcaccagtggcagcaccgggacacctaagggagttgttaattctcactccagtttccttgccgcgatgccagtctatctgaaagcccttgaattggataataactctagagttttccaattcgctagttacgcgttcgacgtgaccatttttgatgcactgatgaccctagtagcaggagggtgcgtatgcgtaccaagcaatgcagataggtcaagtgatctgacctcagccattcagcactttggcaccacccacctttcagtgactcccacggtggccaggatcttagatccgcaggacttccccagtcttaagacgattgtgcttggaggggaactaagcgcgtctgatgaattgctaaagtgggttaataacgtcagagtgatcagattatacggcgcatctgaatgtacggtgatgtccattcaatgcacctccggacccgccagctcaattaagactatcaattatgaaactggaaactgttgctgggttgttaacccacagaatcacgaacaattacgtcctttgggtgcggttggtgagctattggtggaaggtgcagtcgtaggtcgtgggtatttagatgatgccagccaaacctctgagacattcattgaggcacctgcctggttacaagagttgagacaggggagtagcactgtctacaaatcaggtgatcttgtgcgtatcgcagcagacaagtctgtacaatttgtatgtaggaaaagtacacaggtgaaattaagaggccaacgtatcgaattgggtgaggttgagcaccatgtaaggcttgcgatcccctcagctacagagtgcgtggtcgagctaattacgaatcctgatgccagtagaccccccatgctaatggcgttcgttttgagcgatacggacgcttcaacttcaagtattacggcgaggcgtaacgcaacgtctgacgccgtattcgcagagccttctgcatccttccgttcacagatagcgtctattaccagcaagctaagggatgccctaccctcatatatggtgccatccgtgatcctacctttacgtattatgccactgacctcaacggacaaaataaaccgtaaacttctgggtcaattggccgctgctttatctcgtgaggaccttcaactataccaggcacagcaaaccacgtacagggcgcccagtaatgatatcgaggaggcatttcagaggttttttgcgcaagtgctgggtttgtctttggaccaaataggggcagatgaccattttttttcactgggaggggattccctgactgcaatgagattagccgcgatggccaggaaggcaaagttcgacttaaccgttcaaaatgtattcgatcacccagaactatctgagttggctcgtcatacgaaactggtggcggatgagtcccaggagttcccaccgcctttcacccttgtagccggttccaaacaggggatagttcgtgatgccgccaggcaatgcagacttcccagtagggtgatcgaagatgtgtacccgtgtactccgttgcaaaagggattacttgccgagacaatgagggacgcagccgcgtttgtcgctaaaatcgaggtgccgttgccgcgtgacgttgacctggacagactgagacatgcttgggctgctgtcgcaaaagctaacccaatcctacgtacccgtatgatcttttcaccttcttacgggatgctacaagtagttgtaagagaagatattccctggatagaaagcgacgatgtcgagtctcaagaattagtggcagtggggagaagccttgtacagcttatcctacgtagacgtccatccaccgcgctttttttgcatattcaccacgcagtgtacgatggatactccttgcctttgatgttcgcgcaactaaacaacgcgtatcacggagagaccttagcatttagacctgctagtgcatttatcaggtaccttgccaccatgccagatgccacggactactggcagtcaatgtgccaaggtctggaatccccctcattcccggcgctaccacacagtagccaccgtcctcaccccgactctaaggctacgcacacggtgtgcgtagcaagcccacaagcaagagagtacacgccaaacactcacgtccgtctggcctgggcgatcacgcaggcacacgagcaaggccttttagacgtattttatggaaccgtagtttccggacgtaatgctcctgtggatcagatagaatccatgctgatcccaaccgtagccacagtaccgtgtaggatctccttggatgtcgactcaccggttcgtaaaatcttgcaccgtatacaggatgtggccactagaggaatcccgtttgagcagatcggtctagccgaaatttcacatttgggaaaggacgcagcccatgcttgcagctttcagaccttactgttgatgcaacctacggcagttgagcaaaatgaaaatgattttttcaacacttccacatcagatgccaactatcgtgcggatgctacttacgccattaatttgttttgcacgctagaaaatcaggacctttccgtgacagctctgtatgatggtaatattgttagtaccgatactatgcagagattgctacaaaacttaggcaagagcatgcaggagattcatgcagctccgaggacattaatcggtgacatcctgaaaagcctacatagtaggctt

SEQ ID NO: 85 - DNA, 5382 bp, synthetic construct
  source 1..5382: mol_type = other DNA
atgtcatccaacagtataaagttaaaatttaattgtaaggattctctatggcaagaacacataggtgccggggacttttcatttgcggcgatcttccttacgacttggggcatagtactgagagcatatctggacacggacgatgtcttctttgaatatggcctgagtaggtgtgagccagttagtcaaagggatgggatagatgaacttgtgcccgataggccgtttaaccttaaatttgatggtagtatgaccgtcaaagatactatacgtagggccgatagcgtcgtgtacggcacgccactggtgcaggacggcttggagcacgagacccacagagagaccgatccaaaacgtgagaccttttacagcacctgcatgctgttcctagatggtccgaaaagccctattgacgccgacgtcgttgagggtaccattttggacgacatcgaattcgagatgaaaccggttgttaactatgacatggtcgtttacgtactttctgggaaagtgtgttatctgtcctacaacgtgactagggtctctgatgaccaagctgctcatgtggctgcaacgtttgagactgttgcgaaatgtattgcagatgtgcctcacagacttgtgcaagaggttgaaagcttaagtcagctggacgttgacaggcttaaaacgtggaatgcttaccagcctatcgcagtcgaaacttgttatcaggacctgtttagacagagatgcgatttgcacccgaactcaccagcagtaatagcgtgggacggcagttttacgtatgatgaacttgaccacttttcctccttgcttgctactagactgcaagctgccgggatagggcctgacgtctttgtgaccatatgcgctactagatgcaggtggataccagttgccatgttaggaattattaaagctaggggagcgttctgcgctctggacttgagccatccccttgacagactgaaggatatctgcgatgccttaaaatctacgattaccattacaacacccactgattcaaatatagcgagaaaattagcttccactgtcatagtcataggaggagacgcgcctgtagagagtgataggataactccgatgaacgacaggcccaaacccatcaacggacacccaaccaatgcattgtatagcgtatttacctcaggcagctccggtaaacctaagggtgttgtagttgaacacaggagcttcgtgtctagtgcgctagctagtatccagccattagacatccgtccgcacgatagagtgctgcatttctctgcatatgcctttgatatctccgtcttcgaagtattgaccccccttatcagtggtgccacaattgcgatcccttcagagaagcgtcgtaaggaatcattaacgcatgccgtgcaggaattgggagccacctgggcacttttaacgcccacagttgcgaggttgtacgatcctgatgaattcccaagtctacgtacattagcccttggaggtgagttggcccaggcttccgatatagctctatggcagtctaagaacgttgtggttatttataatcccgccgagtgctgcccaataggtgtgtctggccccgcctgtccagctgacggtaaattccttggttggtcacatacatgccaaagagcatggatagttgacccaagggaccacgataaacttcctccaataggggcagtaggagagttgttgattgagggtcccgtcgttgcccgttgttatgcacatgaccctaatttttcttcacccgatagccccttcattcaatctacgcctagctggatgctaagattgaggtccaatacgccctcaggtactagactgtataggacaggtgacttggcaagatatggctcagatgctagcctatattatatggggagaaaggactctcagatcaagattagaggccaaaggacggaaccgggggagatcgagagcaacctacattcaatactgtcaaaagataagttaggtgtggctattgttgtattagagttacgtgggtcatccaagattattgcgtttgtttccaaggacacaggtggcctaggcggcgacagtaatacggtaggacaattacgtatagaggcagccacggaagagacggatgtgtgtataaccaaagctactagcaagctacacagcataatgccagcgtatatggtaccaagcgctttcctgtccgtcaattacattccgatttcaaggtccggcaaaattgacagaactcgtctaaagagtttcgcgctgtcattaccccaggagacactgttacgtgtaaacaatgggctggaaacgggcgatctacccgagtctaacgaggaacataggttacaaaggatgtatagtctagtgctaggaatatcccgtgacaaagtgggaatggagtcagattttttccgtttaggtggagattcattacaagctatgaagctattggctcttgcacccaaagaggggcttacagacatatcttacgaagacatcttcagatatccgaggcttaaggacttagcccgtaaagcttctcaatctgttactataaagaaagatggttttggagagaactccagtgtcattcacccgttttccttggtcatagatggccagtcattgatcgatatggccgctaagcaatgcgacatcgagagggattctattgaggacatttatccttgtacacccatgcaggccagcataataagtctggccgtgaaaggaaaaataatgcctttcttaacattcggactggccttgagagatcatgtagacaccaaaagagtcaaagacacctggcacgcggcctaccgtgctaactcacttttgcgtacacgtataatcgtgtgcgcagaaactggacagttatatcaagtcgttgtggggggggacatcttctgggacgatgacgagtgtggcaatttcgcacaaccaaaaagcggtccatcagccagcattggaggaccgctagttcgtatgaaactggtcgagggacaactatctatcgcaattcatagagccttgtacgataattggagtatacgtcagcttttgaacgacatatccggggcgtataacggtctaactctacctagtagacccagttttaactgttacgtgtcctatgccgcaaggagcttagaggctgctagtagcttctgtaatgccgagctaggcgatagcgatctggatgctgctaaatacccggagccagtatcccagaattcacacacaaatttcagggcatggttgggcatacgtgtgttcacttgccaaaaggaaagcatagatgtactagcaagcgaattccagttggcttgggctatgatcgcgtatgctagaactaacaagaaggatgtcgtctttggagtactgagctctggtcgttctaacgctagcaaagacactaaagagatcatgggtccgattgccactgttacgcctttaagagtaacaattgacggtacccaggatgtaggtggtgcattagaggaactgcaatacaggcaagaagaacaggcgatgtatactcacctagggctgaggcgtataggacagctggggagaaatgctgctgcggcgtgccagatacaaaccgtccttatcgttgaaccggatcttcctgaccttaggggtgtgtggttttccaatgacgcaacattgcctaaccatagcgatgctgatgcttccaactacagattaactatcaaatgcgtagtggggcctgattgtaccgacatttttgccatttttgaccaccaatcattaccaatcatggaggtcaaggaaattcttagtcaattcgagcatattcttggacaaatacacggtaaagaagcttctcaactatcagttgcaagcatcgacacggtgaactttaaagactgggatacacttcataagcttacagaaatgccgtctgtctgcaggaatggcctattactatctgatccaactattcttccacaggggcaaatgaaaacattctcagccgtggaggaagcagcagcgcattgtgcttttcaagattcccttcaagaagcaagtatagctagagatgcaaaaatgcaacccaaggagccgctgtcctcagcggatctgattagcgaaataaaccgttacgatctagccattatgagatcacgtccctccccagagagtattttgctttcagaattagccttaacaggggatagtcattcatctggaacccatacagtctttgtcacgggggctaacggctttatcggtactcaaattcttcgtcattgtcttgaagacccaaccatcgacagggtcatagcgttggtaagaggaagttcagcaaacgaagccagaagtaggacagaagaaagtgcgaggcgtgcccagtggtggagcgactgtcacagtcaaaaacttgaggtgtggcctggtgacctagcgatgccgcaccttggactaaacgagactcattggaggcgtctggcagataggactaccattaatgcgattatccataacggggcgtctgttcattggctaaaaaggtacgctgatctagaagccacaaatgtcggcgcgacggcgcagctgcttcagctggcagtggccaacccaaggctgggctttgtatacgtcagtagcggaaggtacactgatccgaatgccgagtcagaggaaccagcggcggcaaatgtcgcagctacagccatgccctacagtcaaacgaaattcgtagctgagagcttaatacgtcgtacagctgcccgtttgcctcatgggcaaactcaggtgaggataatctcacttgggcttgttattggagatcctttgactggggtagtcaatgcagacgattatctgtggaggctaatagccacatgcgtacaagctggcgaattcaattcctcagcaggcagtgagtggatgccaatctctgatgtaacctctaccgctctagcgatagtccagactgcattaaaccctgcgggtgtccccgccacgataaagccgattactggcgggctgatgtggagcgaaatttgggatctagtaacggatatgggatacgacatggagcctagacctgaaagtgagtggatggccactgtgagaagggatctggaacgtgagcaagaaagacatccgctgtggacactatcccatcttgtagaaagtagaagtcagctaaacactgacgccggggcaggccccgcatgggcggatgcttggagaggagatgaggctactacaaggaatttaaagactgcttttcgtaggtcattaagattccttggcgaggtcggtttcttaccaggtcaaaagggtcaaaataccgacggcgaagtaaatggtagggcgtttacgcgtgcatgg

SEQ ID NO: 86 - DNA, 10773 bp, synthetic construct
  source 1..10773: mol_type = other DNA
atgtcatccggttctaccacagtagagcccgtaggtagcacttacgcgaggccgaaaccccttggtaacctagatcacaggatcgagcaactgctgggttcagagagcgtcttaaatgaatcacccggccattcaccaaacgcttacggcattccttacacagcacgtaatcttacgcttgaagacgtggaccactgcaagagtctaacgaataaagacttgtcattagaattctggaaatcccatttgcaaactgcaaaaccgtgtatccttaccaggctaaaacccggcacgggttcctctattctaagttcacgtcagagtgttaaagtcgatctaggcggtgtgaataccacctgtaggaatcttttcgatagtaccggtatctcccttatgaccgtatttaaagtggcatggggtatggtccttagcacatatactgggtcagatcatgtgtgtttcgggtacggcgccgccaggccttccatgtccgtcgacggcgagcaaacgactattaagccccttattaaaatgctaccgtgtgacatgctttttaagggagggaccacacttttggggacagtgcataaggcagagtccgactttatatgcactattccataccaaagtgtatctattgccgaaatacaccacggtatcggaacggaaagcggtgctttatttaacaccagtattattttttggcctcaagaagactttagtgacaacccgatcaccgaggttttatcacagaaggttacggaaacggagagtctaactgagtgcgacataattatgcacgttgacagtaaacaacaatgctacttatcatattgggactattttttatccgacgagcaggcaaaccatttagccggggcacttaatgttgctatgaaaagtatatttgaaagccctcataggccggtgggccaggtcgagatgttttcatatcttgatcaacagaaactactacagtggaacgggcaggctccaatagcgagtgagagctgccttggcgacttaataggtcaaaactgtcactccaggccggactccctggcggttgatagttgggacggctttttcacttaccaggaacttgatagactaagtagtactttagctaataaattgagggcggccggagtagggcctgacgtcttcgtaacagcttgcttcgacaggtgcaaatggatgcccgtcgccatgctgggtattataaaggcgggtggggccatatgtgctctggatccatcatacccgctgggccgtttaacgggtatgtgccaactactgaaatctacagttgttcttacgactgcaaacaatgcacagcaggctgaacagctagccctaactactatcgtattgggaaacgatttgtggaccggcgacagctatgatgagcaagagaggcaagctcccctgagtaatgtctgcccgtccaatgcactttatgcggtttttaccagcgggagcaccggtaagcctaagggggtggttgtggagcaccgttcattttcttcctgcgcgctggcgagcctaaagccattagacatacgtcctcacgatagagtgctacacattagttcctatgcattcgacatcagcatattcgaaactctggccacactgactgcgggtgcttccgtggcgatacctagcgagaaagcccgtcgtgaggacctgccgggagccatgagggaacttcaggccacctgggcatttcttacacctacggttgcaaggatgtaccagcccaaggactttccgtcactacgtaccctatgccttggtggtgaggcaattcacacctctgatattggtctttgggcctcaaagaacttagtgacaggttacaatcccgccgagagctgtccacttgggatctctgggcctgctgataagtcagctgcgagttttctaggctggtcattttctagtcaagcatcctggatcgtcgatccacgtgattaccaaaaacttgctcctatcggggctgttggcgaattgatgatagaaggccccgcggtggccaggggttatatacacgatctaacttgctctcatccggattcacccttcgtcttacctcctcctcaatggctgtctagattcagagcaagcacatcacaggatactagactatataggactggtgacttagttcagtatggcagtaacggctctgtccacttcattggaagaaaagatctacaggttaaggttcacgggcagcgtgttgaattgtctgagattgaattccagctgcataagaccctgctaccgcttgattgcaaagtcgtagtggatgcggttacatttacgggtcacacgtcagtgatagccttcatcacagctgcagaacactccgatctagagaacgaggatgatgcagttaggtcccctgatgttaaagtgatcacgcaagactttgaaataagggcagcggatgccgcgacaaaattgcagtccatcttaccaaggcatatggtaccaaccatctatttgccagtaggccacatacccatgtctagaagtgggaaaatagaccgtcagaagcttaggtccctagcgttgagccttcctagggaaactctgtaccacattggaaggcagccaggtccaggagagatcgtcgctacagacgttgagagacgtcttcaactattgttcgcacaagtgctggatctgtctccggaaaaaataaaagctgattcagactttttccgtcttggcggagattccatctatgctatgaaattacttgctttggccccacaacaaggtctgagggacctgacttacgaggagatttttcgtcatccaaaacttagggatttggcagcagcttctagtagcttcagcaatatatttctagctattcccgaagacttaggtcccgcaccattttcactggcgagggatgctgattcacttaccaaaatcgcgagcgagcagtgcggtgttgccgtaggagacattgaggatatctacccttgtaccagtttacaggagtctctgatagccagcacagctcacgaccaggatgcgtatgtaggcgttcaatctttcacgctgaatgacgacatagatacaacgagactgaaaatggcgtggaagatgacaagtgaaggacacccgatattaaggaccaggattatccaaacagacagtggtgctccctatcaggctgtaattagaggtccacttagttggtcagaggagagcagtagcgatgatctgccgcctcaattccaaccatctattggccttgggacgccgttagtccagctttgcttaacaaactccaggcttcttgtcgcaatgcatcacgcgctatacgatggctggtccttgcctctgctgttggcagaagttgatcaggcctatagacaattaagcgtgagacaactgcccccatttaaccgttacgtgaagcatgttaccgagaccgttgactcagcagcaagtttttggaaggccgagctgcaggatgccgaccccgtggactttccaccgctgccacatttgaattataaaccggaaccgagagccttgctaacgaagtccataactttaactgcccacacgaacagccaacataacgtcaccagagcaacagagttacagttagcctgggcactgaccagtcacacttacactaacagccaagatgttgtattcggtataatatcttcaggtcgtggtgcccctgtggcaggcatcgagcgtatgttgggaccgacttttgcgtctaccccgttgagggtgccgctagaccccgcccaggacgtcagagaagccctagaggagctacagtacagactagcggaacaaactaagtacgtacaaatcggactacaacgtattagacagcagggtcccaatgctgcggctgcgtgcagctttcaaactatgttagtggtagagcccaaccagcctgttaaaactcaaagcgcttggttcagtagacacgaattcctttcagaattaacgagattttcaagccattctctgacattgaggtgcaaactacttgcgggatctgtagaggtaacggctatctacgatcaactggttgtacccgatgcccaaatgcagagaattttgagtcaattccagcatattttaacacagatccaaggtatagggtcaagggatacgacaataggcgatattgacaggttatcatctggcgactggaacgaactgcaggcgtggaattctacgcttccccctggtctggagttgtccgttcaccagatggtgcaggcaaaggcgcagatgcagccggaagccctggcgatacacagttgggatggcgacctgacctacaaagagttggaagattatgccaaggggttggcaaacaggctacatgcgttaggagtccgtcctaacacatttgtcgcgatataccttcaaaaaagtttgtgggtcgttgtggcgcagctagcagttctgatcgcaggggctgcattcaccactttggagacgtcacaaccaatcaacagactgagagatgtatgtcgtaccgtacaaccaactgtagtactgacatctgataagctgaggctttctggcgctgatctggaagtccccgcaccgctgttggtaataaatcaacaacttctattggaagaaccgggaagtcacagtaggccattggagaaccataccatcatggcttccgatgccatgtatagtattgcaacatctggaactactggcaagcctaaagttgtagtgatcgaacatcaagcgttcttagctaacagcaagcacctgatcgacaggtgggggtttaccgccgattcaagggtcttacagtttgccggctactcctttgacgcaatgatcgtcgagcattttattacacttttggccggaggatgcatttgcataccctcttcattcgatcgtgacaataggttggccacatgcatagtagaaatgcgtgtgaactgggcgatgctaacgagttcagtgattcccctttttactcctgccgctgtccccacgctgcaaactctggtccaagcgggcgagccgatgcaccagggaattacagactgctgggcgtcccatgtgcaactgtttaatgcatacggccctactgagtgtagcgttatttctactactagtaatgtaatcaatccagacgcgcgtaacccgaggaacatcggatttaccaccgggggggtttgctggatcgtcgacccggagaacccggaaagtccccccgttcctatcggggcagagggagaattaatcatcgaaggcgctatactagccaggggttatctgggtgatcgtgtaagaactgctgcagcgttcacaccgcgtccgggttggctagatgatttccgtggatcttcaggagataacagaatttatcgtaccggcgacatcgtcagatatgatcccgatggttcaatttcctatgtaagaaggaaggactctcaagtaaaactgcgtggacaacgtgtagaattactagacgtagaacatcaccttcagaattgttttcctggcgcgttgcaggttgtggcagacattgtaacggtaccaaatactcaaagttcagccttggttgcgctggtccttgctacacctacctcaagttcttccgttgccatcgagtcctgtagcaatgatgatcaggcgactactgcgcacggattgctgctattagctaacaatccgcaattcctaatcgatgcgagtgcggctgagcttgctctacaagatagggtgccgagctacatggtcccgtccctttttattccgacctctcgttttccgagagatgtctccggtaaagttaatagaggtgaaattacccgtagcctggcggcattaagtagacaagagtgggacggatacgtaagcacaaacagagtagctcctaacagtggtctggagcgtgagttacaaaagatatgggctcgtatcttaaacattcctcctgacacgatcggcgtacacgactccttcttcaggcttggcggagatagtataacctgcatgcaagtggcggctcagtgctccaggacaggcattctaataacagtcaaggacatttttaaaagacgtacaatagaaaagttggccgcagcagcggtggtcgtacaatgcccagagagcagtactactgaaagagtaaatacggcagaggcaaaattttccttttatggacccggacaactggaggagtacatgatccaaattcagccgcagcttggggaaggacaagttgtagaagacatctacccgtgctctccaatacaaagaggaatcttgatgtcacacgctagaaattctggcaattatgaagaagttatacaatggaaagttataagtagggcacctgttgacgtttgccgtttacgtgatgcctgggcgcaggtagtcgatagacacgcagtactacgtacgttattcctacatgtctgtaaagagaattacctagatcaggttgttctaagatcacacagccctatggtcctagtctacaacgagggcgaagagccggtcaatcccgtctcaacggggtgctctcagccgatgcatcacctaagagtgaaaagatcaagcgcgggcggtataacggtacgtttacatattaaccacgccctagtagacggggcttccttattcatcataaggagagatctggctatggcttatgaaggacgtctagcctccagcagagcttcttcaccatatagagattacattgcgtatctgcaaaactgccactctcagatacaaagcaacgagtactggcaaagttatatggaaggtactgcgccgtgtttattcccgactttaaagaatgcgggagcacaagattcccaacaacctttcgaggcctttaccctacagttgggagcaacggcagaccttaatcagttttgtgagaatcatcgtttagctcttacgtcagttctgcatgttgtgtgggccatggtcgttcagcgttacacggcgatggacgaagtgtgcttcgggtatatgactagtggcagacacgtccccgtagccggtgtacaagatattgtggggcctttgttcaacatgcttgttgcaagggttggattgccgcatgacgctaccttactgtctgtgatgcagaagtaccacgataattttcttatatccttagatcaccaacatcaatctctagccgaaacgcttcattccatcggctccgcatccggggaacttttcaacacattggtgagcatattcaatgaccaacgtgagggcgaaccagctcataaatcctccgcagtaacgctggtcggggatgacattcatagtagatccgaatacgctattaccttgaacgttttaatgttagctgaccaagttcatattcaacttagctatcacacatccttgctttctgataactacgcgcgtatgattgccgagacttttaggcatgtattagctacagtgcttggccaaccacaattgaggctgaatgaaatagaaatgctggacgaagagcataggtcaggactatatgggaggaatcacgcggccgtcccgtcattcgactcatgcatacattatacgattcaccagcgttgcttagagtcacccgatagcccggccatttgtgcatgggacggcgattttagttatcgtagattagaccagctaagctcaagtttagccgaagaactgatagggcatggcgtaggtgtagaaatgaccatacctgttctactggaaaaaacgtgttggacccccgtagccatgcttgcggtattaaagtctggtgcgtccttcgtgctaatggatgccagtcatccacttgggaggctacaaacaatttgtgaggccatcaatcccccagtaatcctggcaagcccacagacaaggtcaaaggccgttgggttaacaagtcacgttatagaagtaacaaataggttgttggaacaagaacaagctgaacagcagcagacatggcccagggtcgttacaaaagggagcaatgcagcgtatgtggtgtttacttctgggtcaaccgggaagccaaaaggggcaattgtcgatcatagctgtttggcaacagcggctgggcatttaccttcccgtatgtatattaattccgcttctagggttctacagtttagctcacacgcttgggacatacccgtgaccgatgttctactaactttgagggtcggtggatgtgtgtgcattccgtctgacgaagaacgtatcgggaacttggcacaggtcgccaataggatgatggtgaattgggctctgctgacgccaactgtagcaaggctggtgaagccggaagattttactcatctgcagacacttgtcttggccggggaggctgtatcatccactgacctgacgacttggcatgataaggttaggttgattcaagggtatggtcctgctgagtgcagcttggtatccacagtgtcagaacctctaaccccatccgacaatccgaggaacatagggcaaccaaacgggtgtgtcgcctgggtcgtacacagggacaaccatcatttattagcgccgagcggggccataggcgaacttgtacttgaaggccctatcgtgtcccgtggatacatcaatgatcccgagcgtagtgctgcagtattcgtggaccctccaacatggttaacaagattaaggggcggtcattctccaacacgtctttataaaacgggcgatcttgtatcagcggggctggacggctgtctgagtttcgttggaagaaaggatgatcaagtcaagataagaggacagagggttgaacttggagaagtggaggcgttagcgagtcaagcctttcccggctcccatgtcgttgtggaaacagttaaagacctttcaagtacaatactggttgcattcattctgcagaaagagactgctcatgcacaaccgagcagcatcagcagcctgttgcaccccccaagccccttattcagagaattgatctcagcggcttcatgctccctgcgtgagaccatgcccagcttcatgattccaacggtattcttacccctggcacacctaccaaaagcaccgaccggcaaaactgataggaagttcttgagaggtcatgttgcatccctgtcacgtatggaactagaagcctattccatcgtggacgccactggaagggcaccatctaccccccttgagaccaggctacaggagctggtcggaagggtgcttcatagatcccctgaatcaatcccgcttgatgaagacttgttcacgtttggcttggattctttaaccgctatgacgctggcaactttagttagggaggatgggttggcgatttctgtacccaccatctttcagagacctaggctgtccgaacttgcagttgtgctaaatcaagaacagcaaatcaagcaaggacagttcctggctccgccgcctaacgcacttatggcaagtatggatgaactatgcgcccagtggcaactagataggtcccaggtggtcaatatcgctcctaccacgtattatcaacgtggctctttggcgtcacatcataccaactttattgcgttacatttcagccagcctcttgaccctattgcatttaggaacgcagtggttggcctggtgcaaaagcacgccatattgagaacggcattcgttccctttagagaaactttcgtacaactgatcctaagggatttcgacctacctgttcaagagattaggacagacgaggatgatccctccgtcgtggcagagtctttctgcagagaagcagatagggtcccggttagctttggcacgcctatcacccagttatatatgattctgggacgtgcgggggataggctaagcgctgtgttacgtttgcaacgtgcgcagtatgacggagttgcggtctcctgtatgatcgccgatttgaggtcagccttcgacgaggcaccttcaagtgcattaccgacattggagtatgccgactacgtcattagccgtcgtgcccactcaagtccgccggtgttccaagtctggagggaattattacagggcagctctatgacgtatctggtcccacccactgagtatataaggtcaaccgataggtctcgtaccgaactgcttgtaacctcttcttgtgatatacctatgccggatacgaagggtggaatcacgatggctaccgtgattaaaactgcctgggcgctgtgtcttgcgaggcaaacacaatcacaggatgtggtcttcgcccaacttgttaggaaccgtcacttagcgattgctggaatcgatagaacggtaggcccttgcattaactatgtacccgtacgtgctagcctgaacctggactggactgcgaaggaatttttgcactgggtacaaagacagcacatcagaacgatgacttgcgatatggccgactgggacgatttggtgatagaaagtacatcctggccaagggatacagagttaggctcagcggtacattacctgagcgcgccagttgcaagcgactacacatttgcaggtgacgtgccgtgtcagttccagatgtatgacttcaagatggtacacacctatccgatggttacttgtttgccatttccgtcagtcggggacagttctcttactgtattaaagatcattctgacatcagcggtttttggacaaggggtcgcggacaggctattatctcttttccgtgatatggtacacaggctaactacgtatccggaatcactggtttccgaactactgattatcggg

SEQ ID NO: 87 - DNA, 4059 bp, synthetic construct
  source 1..4059: mol_type = other DNA
atgtcatcccagagtcaggtcaggctaccgagtaacgggaatccagccttaagttttccccaaaacgaaatcgggggtccggtacattggaagacgtatcttgaagggtgttcaccctgcagcttcccaagcttacgtacggtgtgcccgtcccaatcttggcctgcgtccgtaagcataactataggagacagcatctttagaaggttagaacacttctcagacaagaccggcatatctgtcgcgacggtctttcgtgcgagctggggtttagtcttaagaatctatacaggacaggactccgtatgtttcggagatatgacgacggcgccagggggtatcgatgctatcgggtgtgttggggtatgcagagtggagcttagtgatacagctgtcatctctaaggtactacaacgtatccaagcggcgtccgctaataaactagctatacctcatgtgccccttagcgacgctgtgctaagcaagtgtatgcccagtgcttctttatttaacacgtgtatgctgatcagtggaggtggagagagacccgagaagacgtcctctacattcgagaaagtcgaactgggctacgatgaccaatatgatattatagttcgtggttcagtagaggattccggtgccagcgccgcgctaagctacaggactagtttcctttcagaggagcaggctacatccatagcgaatacatttgaacgtgctgtatctgatctaattggtatagagaatagaatcgggcaaatctgctttctatcagacctggataaaagtcagatatacacgtggaataaggacccgccgcttagagcacactcatgtgtcgacacccttattcacgaaaggtgcctgagtcagccgactgcgagcgcagtcaacgcttgggatggtgaacttagttacgaagaacttaaccatttaagctccaagctaagtaggcatttggtcactttaggcgtgggcgccgaggtgttcgttccgttgtgctttgagaaaagcaggtggactacggttgctatgcttgcggtaattaaggcggggggggcttttgttcttctagacccatctcatcccgcggagaggttgttgtcaatttgccagaaggtcagcgcaagattgattgttgcgagcgcgcaacacgctaaattggcagaggacctggttacatccatcgtcgaagtaggggacgacaaagcggactggctgacagataaaggtatcaaagcccaaactcagacaagaacaagaagatccgcagctccaggcgacgcgttgtatgcggtctttaccagcggcagcacgggcacaccgaagggagtgattatagaacatggatcttttcacgcagccgtatttccgtacaccgaggcagtaggtttgaatcaagagtctagagtattccagtttagctcatatgctttcgacgtcactatttttgacacgctgatgaccttgatcagtggaggttgcgtttgtgtgccttccaatacggaaagatggtccgacgtcgcaaatgcaattcaaaggtttagggtgactcattcttcactgactccgaccgtggcaagaatactggatcctaaggatgtgcttaccttaaggacactagtcctgggaggcgagaaacttgttacatctgatataacaaaatgggtagaccaggttagattagttcatttatatggcgctagcgaatgtcccatcatgagtatccaaagcatgactggagtagcttctgactttcagactacggaccatgcgacgggatctaattgttggattgtagacccaaacaaccatgatagactggtgcctattggcacaataggcgagttggtgatcgaaggaacaattgttggaagagggtatcttgatgatcccgagaagtcttctgccaccttcattcgtccgcctggctggctatgccaagtaaggggtagtgggtaccatagtgctgtgtataaatccggggatttagtgcagtacacggcagatggttccctgaggtatatagggaggaaagacacccaggtaaaattacgtgggcagagagtcgaactgggtgaggtagagcaccatgtaaaacttaccttcccaaatgccaccgatgtggttgtggaactagtcgtcacgattcacgcgtctagtagccgtgctcctattttagtggcgtttgtacttatctcacatgaagcggatccagacccagagtctattaggataggacgtggcgagggcttgtcacagatactgagcgagccgactgatcgtttttgctcccaaatcccaattgtccaaagccagttgcaacaaagtcttcctagttacatggtccctggcatatttttgccgttgatgacgctaccgcttacatcaaccgacaagatcaataggaaactactgagagaacttgctggagctctatctagagaggaattggagagctaccagccatctactggtcctgtgagggccccacagactacgacggagaagttacttcagcaatattttgcgcgtgttctaaatatcccggtggaacaggtaggggcagatgatcattttttccaacgtgggggtgattctcttacagccatgaaattagtcgccatggctaggaaagataagcacaaactaacggtgcaagatatctttgattcccctaggttgagcgccctagcgtgcgtggtgagaagcggtaaggtggatgggaataaagagccaccgcttgagccattttctttagttaacaagcacagagatatcatcagagcggcggcccagcagtgtcagctgccagtcagagtgatcgaagatgtttacccatgtaccccccttcaaaggggcctaatttcagaaacccttcgtgattcaaaagccttcattgcacacctggccgtgagtctgcctccagatattgacctacaaaggttgcaagaagcatggactactgttgcaaacgccaaccctatcctaagaacgaggatggtgcttagcgcgagtcacggcttgttacaggttgtcgttagggaagacatcagatggatagtttcaaagaatgctgaagctcaggatttctttgtcggtgtgggaaagcctctagtccagcttgtgctgtgttgtcatagggaggggaatgaggtgcctgttcaactattgttaatggtccaccacgccgtatacgacggatatacactacccttaatcttcgagcaagtcaaagctgcgtataatggcggaactttagcgccaaggccggccgttcccttcattcgttatgttcagtccatcccagacggaacaggatattggaattctttgatggctaatttacaaactccaatatttccggctcttcctagcaaatcctatcagccattgccaaatgccatcatgaggcatacgatcattactccaggaagtcaccgtcagtacacccccagcacctacgtacgtctagcatgggctatcacgcaagcgtatcaccagggcacatgcgacatctttttcggtacggaagtcagtggaaggaacgccccggtaactgacatcgaattgatgactattccgacggtcgccactattccgtgtagggtcacgcttgacttccagtcccttgtacaaagtgcgttacgtaaaatacaagacgatgccatatcaggtatcccctttgagcagttgggtttgccgcatattcgtaggttgggggaacatgcaatgttagcttgttctttccaaacgttgctttcaatccagccggctcttccgccatccacggacgcctggtttgagcagccaggtagcaccatagactatagagctaacgcgacatacgccatcaacctgtttttcggtcttgaaggcgacaaacttaaggctaccgccctttatgactttaatgttgtgaagaaagacaaaatgcagtctatgttagtcgactttgggaacattttacagactatgcataaatcaccgaacagcctaataagagacatcttagctgtaccgcaacaaatgaggggacccataaacagcgcatctttg

SEQ ID NO: 88 - DNA, 3927 bp, synthetic construct
  source 1..3927: mol_type = other DNA
atgtcatccgcaagctttaccgcatggaaaacctatttggaggggtacagtccctgtagtttcccggagctggtcgacgctgagacttcaaaacctggcttggccagtgtttcaaccacgttgaaggactcccatgacaggttacagtcatttgctaatgagcactccaacaacgatgttgtggcggtcttttgtgcagcctggagcttggtattacgtgcatacactggtcaagataccgtctgctttggccgtgcgacacgtaatattaatgcctgtaggatcgattttacgtctgatacttctatactacgtatcttacagactctgcaaacagcttgtgcggaaaagaggttgacaagcgacgttcccgtgagccaactatccttagagactggtgatctaattacaagttacttcaacacctgcgtcctgtcccccaaccaggagaggatccagcagggtgaatctgacataatcgagcgtggtagcactggtttagacctgttcgggatcgtagttatctttgaagtgaaggaaaatgtcgcgcgtgtgacggtcagatatcaaacttccttgttacaagagaaccaggctatctctgtgatcaacacgcttgatcaggctatcactgaaattattaatcataaggatcaaatagacgatataagtctattaagcgcagtggacaagtcccagatctatagttggaatgctaaacttgcccatcatccagggtatcgtgtggacgagctaataggcgaaagatgtcagttatcccccagtgcgttagcagtatccgcttgggacggcgagttgtcttacaaaggtttggacgaggcgagctcctctttggctcaacatctgactgcacttgggatctgtccggaggtgtttgtacctgtctgttttgaaaaatccaggtgggcgattgttgcaatactaggggttatgaaagctggcggagctttcgttctgctggatcccagccatccacacaaaagacttcttgatatctgcaataaggttagcgctaaactaatagtttgcagtacgcagcacgttaaattggcagcttcattggctccgttgatagtggaattaggagacgatgatgcgaaacgtaattggcatgtcgaggatttccaaaaatctgattccctggttactcctggaactgccctttatgccgtttttacaagcggttctacaggaaccccaaaaggtgttgttagcgagcatacgagttttctggcggcaatgcctacatatattaagtctttaggcctaaatcaagagtctagagtttttcagttcgcctcatatgcgttcgatgtaacaatcttcgatactcttatgacgttgacagtggggggttgcgtatgcgtgcccagtgacactgagagaagcaatgatttagcacatgctatccaacatttcagagctacccatatgtccttgacgccaacggttgccaggatcctggatcctgctgattttcctaccctgcagacgcttacccttgggggggagaagctggtcacatccgaattttctaaatgggttgaccatgtacgtgtagtccatctttacggagcgacggaatgctcaatgatgagcattcaatgcattactggaacctcatcacacttaaagactattaatcatgaaaccgggtctgcgtgttgggttgtggatcccgagaaccacgatagactgcaagctataggtgcaattggagagttaatggtggaaggggaaatcgtgggtaggggctatttagacgacccagtgcaaacgagtgagacatttataccaccaccgggttggttgcgtaaacttagaggcaataattgcaaccagagcgttaagatatataaatccggagatttagtccgttatgccgctaacggctccttgcagtttatatgtagaaagaatacacaaattaagttgcgtggacagcgtatcgagttgggtgaagtagagcatcatctaaagttagctttgccgagggcgcgttttgtactagcggagctaattaccatgccagattcaagtaggcctccgatgttaatggcattcgtccgtcctgaggataattcttcaagcgattctttaactggaggaagagaggcggccttgacaacaatattcgcggagccaacggatgagttccgttcccaggtaccggcgactttatcaaaactacaaaactcattgccctcttatatggtacctagcgctataattcctctggcggtcatcccactgactgggactgacaaagtaaatagaaagatattaagacagctggcggcagagttaccacgtgagcaattggaaagatatcagagtactgttcgttcataccttgcacccactacaaacgtcgagaaagcgctgcagaaatactttggcgaggttttggacttaccactggagcaagtatgggctgacgatcatttcttctctttaggcggtgattccctgaccgctatgaaattagtctctatggccagaagggataactatcgtctaacagtgaagaacgtgtttaacagcccccaactgtctgatctagcaaagctaattaaaactacaaaaaacgaggatgacgaaaagctgcaaccatttgcactgattgataaaaagcaagatgttatccgtgctgcagcgcagcaatgccaccttcctagaagggctattgaagatatttatccgtgtacttcattgcagaagggattaatttcagagacaatgcgtgaccccagtgcattcattgcaaaattgcagcttccacttaggtctcatatcgatatctcccgtcttgagcaggcgtggtccgacgtcgcaaaggctaacccgatactaaggacgcgtatgatattaagcgcctcctatggtcttttgcaaacagtcgtcagagacaacattccgtggacagtatcagagaacgcggagtgcgaggatcttgatgttgccatcggtaagccgcttgtccaattggtactgtgtagacctgcgcctcgtgagcaaaaccctgcaaaattggtattaatgatgcaccatagtatatacgatggctatactttaccattaataatcatgcagctaagaaccgcatacgaaggtggagttctagaccctaggccagtgagtccgtttatacgttatctagccagtcttcctaattgcgccgattattggatccctgtactaaaggacttaaaggctccagtttttcccgctctgccatcaaaagcatatcatcccctgccgaacagcaccgcaatctatgcggcgccagtttcctctccttcagctaaacaatacaccccgaatacatatgtaaggttggcttgggcgattacacaaagtcaccaccagaagatcagcgacgtttttttcggaacggtggtatctggacgtaatgccccggttgataatatagatctgatgactatcccaactgtggctacaataccctgtagggttacgcttgacacagatagcgtggtaggagatatgctgcaccgtatacagaatgccgctaccgaaggtataccttatgagcagttcggattgtccaatatatctaggttaggagaatacccggcacatgcatgctcttttcaaactcttcttgttatgcaaccggctgcggattgggataccgatgattttttacagatacctaagtctgatgctaattacagagcagacgccacctatgcgataaatttgttctgtgaacttgaggcggagagattggaggttaccgcactgtatgatgagaacgtcgttcatgagatcaaaatgcaacaaattctagttgattttgggaaggccttgagaagcgtgcgtcaaactccaacctccgtggttggtgatgtccttaatattttagacaaatcaagaaaggaa

SEQ ID NO: 89 - DNA, 5544 bp, synthetic construct
  source 1..5544: mol_type = other DNA
atgtcatccaaggacggagagtgcaccttgaagatggtgaacaaagaaggggcgcttgagttctggagttcaaaactgcaggtggcggaaccgtgcatactaactctttccaatcagagtactgccgttcccacgtcttcagcgatcgagtcaatgacgatagacctaggaggcataggtcttccgtggtcagaatgcgtgaataccagggacatatctagctccgctatctttatgactgcctggggtatgatcctgagagcttacacagatagcgacgaggtttgttttggccatgcttttacggctaggcaagggccgatgaacggacacccagctttgtacaggcccagattaaatatcctaccttgttttctacagttcgagggtaaagctactgttttggagacggtaagaaaaactagattatccctagatgacgcgcagtccaaccaggatgtgtcccttgtcgagatttgtaatcatatgggggtcaagaataatgctagcttcaacacttgtatgctttttagcagtgacgagcagacctcacctaatgcggaacccgatgttctggcccaggaggtgacgctaatggagggttgcccccaatacgacatcattatacacgtctccccattaaagcaatgcgtgctactgtatcagaaacactgcctgtccaatgatcaagccgtgcacattgccggtgctctaagggtagcccttaccagcatcatcaactctccacacttagctatccaaaaggtcgaaatctttagcccagtcgacgaaaataagctgttgcagtggaataaaacagcgccaattgcatcagagagctgcttacatgacttgatcaaccaacaatgtgacttgcatcccgattcactggcagtggtcgcgtgggacggctcatttacctataaagaactggatagactaagcaacacattggctcaaaagctatgcttggccgaaattggccctgacgtctttgtcgccatttgtgccgatagatgcaagtggattccggtagccatgcttggcataatcaaggctggcggagcattttgtgcgttagatccagcgcatccaccgagtaggttacgtgaaatgtgcaaagcattaggaagttccatggttattacgacggtggaggacgcgaaaaaggcggatcaactggcgctgacgacgatcgtgctgggagatgactcaatgacggagtctaatgggaccaagttctcaccgggtcgtgcacctccgagcaagacccaaccatccaacgcactatatagtgtgttcactagtggttcttccgggaacccaaagggggtggtcattgaacacagatccttcgcctccacggcgctggccagtttaaagccactagacatcagaccggaagacaggatcttgcattttgcgagttatgccttcgacctttctgtttttgaggtcttgacagcgttaacggcaggagcttcagtcgccattccaagtgagaaagcgagaatggaatgtttgcccagagccgcaacggaactacaagcgacatggacatttctaacacctaccgtcgcgagaatgtatcaaccatccgattttcccactctgcgtaccatttctcttggaggcgagctagtccagacgtccgatatagagctatggggctccaaaaaccttattacgggttataatccggccgagtgctgtccgctaggtatttctggcccggccaatctgtccgaggctaacttcatcggctggccatttgcctcccacgcggcgtgggtagtcgatacgcgtgatttccagaagcttgtccccgtcggagccatcggagaattgataatcgaaggtcccgctgttgcgcgttgctatctacatgataagaacaacgcactaccagatagcccctttattacatctcctccccagtggttaacgcgtttcagaggcaaaaccagtccggacgctagattgtatagaactggagatctggtccgttatggttgtaatggggcggtccattacattggtaggaaagacttccagatcaaaatacgtgggcaaagggtggaattgggggaaatcgagtctcagctaaggaacgcctttagcgccggtaatcacagagtcgcggtagaagcagtaaatctttctggatgcactacgcttgtagcgttcatctcagttgactcttccgatagtgaaacgaagcgtgacgcagcaccagctgtgcgtctgcaagacataaaacaagaattggagacatacgtggagggtgtggttactaaattaaggggaattctacctagtagcatgattccatccgcgttccttcccgttacccatatccccgtaagcaaatccggtaaagttgatcacaagcaattacgtagcctagcatccagtttgtcccccgaagtgctgtttaggccaaaaggggcgtcaacgtctggcaccctaccagagactgatgacgagagaaggttgcaagcgctgtttgcactagtgctaggaatttccccatccaatataagagctgatagcgacttttttagattagggggagactccgccctggcgatgaaacttctggccttggtgtcccaagaagaacgttttgatctaacatattccgatatatttcaacacccgagacttagggatatgcttaacgcctccagatcaaggagcagttttatgaataacgatgatgagtcagtatctacagacagtagcctacctcctttctgcctggtaaaggatgcaaacagtcttatcgatattgcgagcgaccagtgcggaataaacaaggagagcatcgaggatatttacccgtgcacttctacgcaggcgaatctgatggcaagcactgtactagatgaggacccttgtttatctataagaagcttcaagttggagggcaacattgataaggcgagactaaaacaagcttggagtatagcaagtaaaggccatccaatcttgaggactcgtatcatccaaagcaatactggggcgttttttcaagtcgtaataaagggagacatatgttggtcagactatacgtattccgatgatagcaattcccaattcaggccgagcttcggcttgggttcaccatccgtccaattatgctttatgaagggtaggttactggtcgctatgcatcatgccttgtatgacgcttggagtctaccaaggctgctaaatgaagttgacggtgcttactgccaactttctgtgcaaagactgttaccattaaactgttatgttaagcatgtggcagaaagtctagacagtgcagcgtctttctggaaagctgaactggaggacgcagacccaacccactttcccgtcctaccgagtggaaaaactgacagagagacgatcccaactacaactaagagcatttcaaccactatgccaattgacagtgagcgtaagtcaaacgttgaatttgaacttcagttagcttgggccattacatgttactcatacacgaagaatgaggacgtgatttttggcgtaaccagttccggcagaaatatagcacttgatggtgtggataaaatgctgggcccgatcttcgctattacaccacttagagtacccatcgatccggaccaggaagtgaccgaggctataggggaccttcaatataggtctgaagaacaagccccatatacacacctaggcttacatcgtatcagtcagctaggacaagaagctgctgctgcttgtcagtttcaaacgttactggtcgtagagccctcagagacagaaatcaaagggacctggttttgtgaacacgaagtgatatacgactctatcgaaccacgtaatcacagtttaactttgaagtgccttgttaatcctggatttgttgacattactgcaatcttccatcaagcgatcctgccagacacacagaagcaggatatgctgcttcgtttccagcacatctttggccagattcacacctcagggacaccggacagtactatattagatatcgacacttctagtccccaagatctttcaggcttgcaacactcatcactgtttgttaaaaatgtcggcagagactacactaccacgaacaagttacacaatcgtgatatattcttgtacaactccacccttaagtctcagatacaccatagcgtgcatatggtagataatgcgttagaggaagcagcccaggctcaaaaatctccggttcccacaggtaaacgtagcgagagccaacagtttgcccaggccagcgaagctgtgaacctagttgcggaaattgatagatacgacctagtcataaccagtgtccagcaggaggagatcagtgactctaaaaggtctccgcaagcgaagaatatgtcctttaatttggaggcggggaagccgatcacagtcttcttaacgggggccagtggatttattgggacggaggttttacgtcaactacttgaaaaccattctatcagaagggtcatcgccctggtgagagggagaagtacacaagaagttcgttctaggacgattgactccgcaaaaaaggctctttggtggacggacggtcacggagagaaattggacgtgtggcctggggatctttcactgccccacttaggcctggatgcagaacattggtctatgttagaagatggaatgacggtggacgccgtgatacacaacggtgcctccgtacactggcttaagaactacacggctctagaagctaccaacgtgagatcaaccgccgaactattgcgtattgcggtgaaaacccctcacgtgaggttcacgtacgtctcatcagggcgttaccaagatcctatcgaggaggttgagaaacaggttgccatgcagttcgctgctggggggataccgtattcccagacaaagttcgttgcagagagcctggtgaggagagccgcagccaggattccaagtgaacattccaatttggctgtggcatcactagggttagttatcgggacgcctacggaaggtgtagtaaactcagacgattacatctggaggctagtggcagcttgtataaaagcaggcgtatgtaatgcagatgagcttgataagtggttcccagtaagcgatgttgcaagaacagcttctacaattatcgacacggttctggagcctgtcaccacgtcagcaccggtaaaacaagtcactggcggtattacttggcgtgagatttggaacattgtgatggaaatgggttataccgtcgagagtaggccagccgtggattggcttagaaccattagacaggatatagaagttgagcaagaaaaacacccgctatggacgcttgcgcatatgattgagggctcaatagataacacagaaccgacctgggctacaagttggcacggtaatgaggcggcctcagcacgtttgaaaggcgcagttaagggctccattgcattcttaggcgagattggcttcattcctaaatctggactagaggttcaggagcaggttcagaagattcaaatcggagcgttttctagatct

SEQ ID NO: 90 - DNA, 1563 bp, synthetic construct
  source 1..1563: mol_type = other DNA
atgtcatccgacttcttacaaagaattttgggcattttgagtagtgaggacgtggccccggcactgttcgcctccagtatcagtgtcttgttcttaatactaagctaccaggttctatacccaccttctaagtacagcgccttccccacaatcaacggaacgagatggctagccttgagcaataggcgtattctagcagagttcgtgacggacgcccaaggtctaatgcgtaatgggcttgaaaaatatgatattttccgtataatatcatcaataggtcccatgaccatattgcatcctaaatatacggacgagatacataatgatcgtcagttaaactttatggcagtccttgcgaaggaaatgttccccaactatcccggcttcgaccttttcagagagggaacggatggatccacggtccttcaagacgccgttaagttcgggagctcacgttgtttaggtaagtctacacaattactgagtgatgagacttctacgttgctgcagaaattgtggggtgatgagccagaatggcacgaggtgacggctaagtctagtgtgcacgacatcatcgctcatctgagtgcattactattttatgggcccgagctatgttcccacaaggaatggctggaagtaaccgacgagtatgcttctgtcggttttctagctgcccgtcagttacgtttgtggccccccatacttagaccgatagcacagtggtttttgcccgcttgtaggaggttgcgttatttagcatctagaacaagagggctgattgagcccgttattgctgctaggcaaaaggagaaggccatctgctactctcatggcagacaacctcctgtttatgacgatgcaatagaatggacggaaagggcagcgaaagggcgtccctacgacgcagccatgagccctcttctgtttagcatcaacgcgcttcacactaccactgatctacttacgcaggtcatcctggatttatccacccaacctgacttgattgtggcgcttagacaagagatattatccgtaaaacctcagcagaacggctggaagaatgctagcctgaatcagttgctattaatggacagcgccataaaggaaagccaacgtctgaagcccacagagtctatcctaatgagacgttacgctatggacgacctgacattggcggatggtaataagattcctaaggggaccgtactagggatcccgatcttcggcatgagagacccaaaaatatatgtagaccccgacatgtacgacggttacagattccagaagatgagagataaaccggggttcgagaacaagtgtcaattagtttctacttcaccgtggcatcttggtttcgggcatggaatccatgcatgtccgggtagattcctagcggcggtccaagtcaagatcatactgtgttatatagtagccaagtatgatttcaaactggctggaggcgcacccccaaaggttcagagcgttggtattgagttaatatctgacacggaggctaggcttgcagtaagaagaaggcaggagatggtaatcgggctagaa

SEQ ID NO: 91 - DNA, 1374 bp, synthetic construct
  source 1..1374: mol_type = other DNA
atgtcatccgacgctcaaaggcttatgcgtaaagggttagagaagtacgacgtattccgtatgatatcatcaattggaccgatgaccatcttacatcccaaatacactgatgaaatccacaacgaccgtcgtcttaacttcatggcagttcttgcgaaggaaatgtttcccaaatacccgggttttgaccttttcagagaagggactgacggatccaccgttttacaggacgccgtaaaatttggaagttcccgttgcctaggcaagtccacccagctgttatcagatgagacttctacactgctgcaaagggtgtggtctgatgagccagaatggcacgaagtcacggccaaaagtagcgtacatgatataattgcacacctaagcgcgctgcttttctatggccccgagttgagtgggcacaaagagtggctggaggttaccgacgagtacgcttcagttggctttctagcggccagggaactaaggctgtggccccagatactacgtccgatagcccagtggtttctaccgagttgcaggagacttagatatcttgcatccaggactcgttctctaattgaaccagtgattgcagctagacaaaaagaaaagactatatgtgcctcacacggcagagaaccacctgtgtatgacgatgcaattgaatggacggagagagcagcaaaaggcaggtcctatgatgcggctatgtccccgttgctgttctctataaatgcgttacatactacaaccgatctgctgacgcaagtaatactggatctgtctacacagcctgatcttatagctgctttacgtcaggaaataataagcgtgaagccgcagcaaaatagatggaaaaacgcctccctaaatcaactactgttgatggattcagctatcaaagagagccagcgtcttaagcccaccgagtcaatccttatgaggcgttacgcaatggatgacttaactcttgcggatggtacgcaaataagcaaggggaccgtactgggaattccaatttttggtatgcgtgatcccaaaatatatgtagatccagacacgtatgacggctatagattccagaagatgcgtggccagcccggtttcgaatcaaaatgtcaacttgtctcaacttccccttggcacttaggctttgggcacggtattcacgcatgtcctggccgtttccttgctgcagtccaggttaagatcattctttgccatattgtggccaagtacgacttcaagctagcgggcggggcgcacccgaaggttcaatctgtaggcattgagttaatctctgatagcgaagcaaggctggcggtaagaaggagacaggaaatggcgattggtctggaaaaggatcaatca

SEQ ID NO: 92 - DNA, 1566 bp, synthetic construct
  source 1..1566: mol_type = other DNA
atgtcatccgatttacttaggaagtttagtggaccgttggtttctacaaatatggaacctgcattgttttcctcaatcgccttctgcttcattttggttattctagtgaatcaagtcttatatccctctagcaagagcagtccattcccgacgatcaacggtcgtgggtttctggagcttaatgataggaggatattaacggatttcgtcatgaatgcccagggtcttatgcagcgtggattgtccaaatatgatatttttcagatgcttacctctattgggcctatgacgatcctacacccaaagtacacaaatgagatttataagaataagaggttaaacttcatggcagtcttagctaaagagatgttccccacttaccccggatttgacttatttagagaaggaacggacggctcaactgtgttgcaagacgctgtgaagtttggctccaccaggtgtctatctcgttccacgcaattcatgagtgaggaagccggtattttgttgaagaaggcgtggggtgatgagcctgaatggcatgatgtcacggccaaaagtagcgtacatgacattatcggacatatatcagcactgttattttacggaccagagctgagtaataataaagaatggctagaagtgaccgaagagtacacaagtactggattcctagccgcgagggagcttcgtctatggccgcaagtacttcaaccattcatacattggtttctgcctagttgtaggcgtttaaggtatctagcccgtagaacccgtggtctgattcagcccgtaatcgccgccagacaacgtgagaacgctcgtagggcctcagaaggtcaggaccttcttacatatgacgatgctatagaatggaccgagcgtgctgcaaaaggccgtccctatgatgcagctatgagtcctcttttgttttctataaacgcactacacacaacaactgacctactaacccaggtattacttgatctttctacacagccacatcttatagaagccctgcgtcaagaatttattacagtaatcccccaacagaatgggtggaaaaataccagtatctcccaattggttttgatggactccgtaattaaggaatcacagaggttaaaacctacagaatcaatcctaatgcgtcgttatgccatgcatgatattaccctagctgacggcacacaaattcctcgtggtaccgttttaggaatacctatattcgggatgagggatcccgagatctatccagacccagacacctatgatggatacaggttcatgaaaatgagagagcagccggggttcaaaggcgtatcacaactagtcgaaacatcacccatgcacctgggtttcggacatggcatacacgcgtgcccggggcgttttcttgctgcaacgcaggtaaagattgtattgagccatatggtaatgaagtatgactttaagttagctggcaccctagaacctaaaatacagagcgtcgggattgagctgatctccgacagtgaggcgacactagcagtgaggcgtaggaaggaacagatgatcctgggcctt

SEQ ID NO: 93 - DNA, 882 bp, synthetic construct
  source 1..882: mol_type = other DNA
atggcagccccagcaagggaactgaggttgtggcctcagattcttagaccgatcgcccagtggtttttaccgtcctgcagaaggttgagatacttggcctcacgtacgagatccctgattgagccagtgattgctgcaaggcaaaaagaaaaagctcgttgcgcttcacacggtagggagcctccggtgtatgatgatgcgatagaatggactgaacgtgccgctaagggtagaagttatgatgctgctatgagccccttgctgttttccattaatgcgttacacacgacaaccgacctgctaactcaggtaatccttgatttaagtacacaacccgatttaatcgcagcccttaggcaggaaatcatttctgtccgtccccagcagaatggctggaaaaacgcgagcttaaaccaattactacttatggactcagccatcaaggagtcccaaaggttgaagccgaccgagagtattctgatgaggaggtacgcgatggatgatctaaccttggcggatggtacacaaatccctaagggcactgttttgggaatccagtcctccatctatgttgatcctgatacatatgatggctacaggtttcaaaaaatgagggatcaaccaggcttcgagtccaagtgtcagttggtctcaaccagcccatggcacttgggcttcggacatgggattcatgcttgtccaggaaggttcttagcggcagtacaagtgaaaataatactataccatattgtggcgaaatacgacttcaagctggctgggggtgcgcatcctaaggtacagagtgttggaattgagttaatttcagacagcgaagccaaattggcagttaggaggagacaagacatggcgatcggtttggaa

SEQ ID NO: 94 - DNA, 1086 bp, synthetic construct
  source 1..1086: mol_type = other DNA
atgtcatccagcgatgaaatggataagtttttagcggaaacttggggagacgaacaagagtggcataaggtcacggcgaaaacagttggcaggaatattattgggagactatcagcgttaatattttacgggaaagagctacgtgacaatgaggaatggttagacgttacagatgaatatgcttacatgggattcatggcggctagggagcttaggttatggccgcaagctctgaggccatttgtccaatggctgttaccctgctgccgtcgtttgagagcattggcgcgtaagacacgtgggctagtcagacctgttctgacagcaagaagaaaagaaaaggcttggtgtgaagcacagggcaaagatctaccggagtatcatgatgctatagagttcatggaacgtgcggcaaaagggaggccatatgatgcggccatgggtcctgtcctattttctgtgaatgccttgcacactacgaccgacttactaacgcaggtactacttgatctgagcgatcagccagcactgattaacgcactaagacaggaggtcttgacagtgatgccgcacagaaatggctggaagagcagcaccttagaaaacctaaaactacttgattccgtaatcaaggaatctcttaggttaaagcccagcgagtctatattaatgagacgttatgccatgagggatgttgccctatcagacgggagtcaaatccgtaagggaacagttctggggatctctaccttccaaatgcgtgatcctgaaatatacaccaaacccgacacgtataacggatacaggttcttcgaaatgagaaagattccagagcttgaaaagaagtgtccgctggttgctacgtcaccggctcatcttggattcggtcatggaatccatgcatgtcctggaagatttcttgcggcgatccaaatcaagatagtgatgtgtcatattattatgaagtatgatataaggcctgctggcgcggctaaggcgaaagttcatgtccatggcatagacttaatctccgaccccgaagcagagattctggttaggaagagagagcaaaacgtggcgttcgtc

SEQ ID NO: 95 - DNA, 1488 bp, synthetic construct
  source 1..1488: mol_type = other DNA
atggcagccaaaaagcaggttacgttcaaaccgtttagactaagtccggtggatcactcccttcccaaggtctatatcttcaagagtctttactttagaggcgtggacgataccggtagcttatcacgtctgcaggacggcatagataggcttatatcatgcttaccatttttatcaggtgaggtcgttccttgcgccgatatacccgacaaagtcggggtgttacaagtacagatgccatgtccctccttgcaggaaattcccatgcttctagttaaatcttaccccaatcacacatggcccgctgcctcaacttcagagagatggcgtaatacggccttgctggatcaaagctatagaccccttccggattttattccgccctctaagcctcgtccagtcctacgtttccaagcgaacttcctggctgacggtcttatgttatgtatgggctataatcatagtgtttttgacggtactggagcgggtaacatactagagatgcttgctgattgttgccgtgctaaccctaatagcatacttgccctacctacgaatggcgacatcgaatccgaactgcgtggattgctttcatcacccggtgtcgccgttgcaaacgcttcccaagaggcctatgcgataaattgcgcacacactgaagtggagcctgaaccctcttctgctatgttatattgttggccatttttgttgtcaagcgaaaagattgagtgccttcaagaggcatgcaacagtcttttaccgcacatagttaggctatattccggtactcagagctcattaataaatcaagacactaactggccccacattctatcctctaatgacgtacttaccgcattgttggcagtttctatagagaaggccagggaggcgacgggagctctgggacatatgagtcgttccttggcgatggccgtgaatctgcgtgagaggttaaagcctatgccaagacattatttaggtaatctggtgactaccgtgtgggtatcacatcaccgtcctgccgtgaaagacctagagacaatggtgctgcctgtacctgcttgcaacaggcatgagatagacagggatgatttgttgtggataacacacgtcgcctttagaatcagactgggacttaatgcgataaatgaagagcacattcgtggtctgatccattacctacactcacaggatgattgggagcaaataggaatacactttactgatcccatctttatatccagttggagacatctaaaggtgtatgaactagactttggcccgacaataggccatgcggagcacttcgagatggacgtaggaacgacagacggggtgtgcgtagtgatgccggccaacacgagagcagtgggtaagactaaaaaagcgccgtgggatattcgtatagtgctaaatcccgaggttctgcaggccctaatagcctctgcgatcttcgggtgggcgatggtcaaggacgcgtccacc

SEQ ID NO: 96 - DNA, 1503 bp, synthetic construct
  source 1..1503: mol_type = other DNA
atggcagccaaaccgagagccacattcaaacccttccgtctatctcctgtagatcattccttacctaaagtatacatcttcaagattttgtatttccaagccgtagatttcacatgctgcctttccctgttacaagatggtgtggaccgtctggtttcatatttaccctttctgacaggtgaagtagcaccgtgtacggcattaaaggggaaaacaggcgtccagcagatacaaatgcccggcccgtcattaaaagaattaccaatgttgttggtgaagtattatccagatagaattttaccgaagtatccgtcacctagtaaaaatagtgctgaatccaaggaaggcacaagcagtccggatcaatcttaccgtcctttaccagacttcatacctgcgtctcagccacgtcctgtgcttaggtttcaggccaacttcctagcagatgggcttgtcctatgtatgggctacaaccatagcgttttcgatggaactggggccggaactatacttgagatgctggctgattgctgccgtgctggggctgcttctgatattagcctaattactacatcagatattgagtctgaattaaggggttttttgtctagtgctagcgtcgccgttccgaaggatagccaagcggagtatgcgataaattgcgctcatacggaggtagagacgcaatctttcccaactatgctgtgcaactatccatttcttctatcatccgataggatagaaagacttagggacgcttgtaatggtttgttaccgtatgttgtagaaacgtatagtcgtgctcaaacttctccagtaaataatgatggcaagtggcccgatttcctatcctccaatgatgtgctaaccgcccttctagcgatttcagttaaaagggccagggaagcggctccctccccgcagccgaggtccaccagtttggctatggctgtaaatcttagggaaaggctaaaatccgtaccgtctcattatttaggtaatttagtgaccacagtgtgggcatcacacctggaaccaccgaaaaggcctgaagcactggtatctacatgctccacctgtgagcaacttgagattgaaatggacgacttattgtggatcgtcgaggttgctttcagagtcaggttgggacttaacagcatagatgaggagcatgcgtgcggcttgattcactatctacattcccaagacgattgggagaagataggtattcatttcacagatcccgtgttcgtgagctcatggagacacctgaaggtttatagattagattttgggccaggtattggtaggattcaaaattttgagatggacgtaggcacctcagacggaaactgcattgttatgccagccgactttaaaaaaatagatgggaagaataagagaccacagtgggaggtcaggctgatcttgaaccaagaagccatggatgcgcttataaacgatagcctgttctcttgggcacgtattaagggtacttttgaa

SEQ ID NO: 97 - DNA, 1047 bp, synthetic construct
  source 1..1047: mol_type = other DNA
atgtcatccggatataaccacagtgtattcgatggtacgggcgcgggaaatgtattagagatccttgctgattgttgcagagcggatcccgcctcaatcgtaagtctaccgacaacgggcgggatagagtctgagcttagggggcttttgagtgacccgggtgttgctgtggcaaatccctgccaagcagcgtatgcgattaattgtgctcatacggaggtagagcccgagagctttcctgctatgttgtgttcttggcctttcctactaagttcagagaaaatcaagtgcttgagagaagcctgtaactcacttctgccttatttggtaagaacgtatagaggctcacagtcaagtatgatttatcccgatcctaattggcctcagattcttagtagcaatgacgtacttacggccttactagcagtaagcgtcgagaaagcgagggaggccaccggagcccaggaacacatgagctcctctctggccatggctgtaaatttaagagaaaggttgaagcctatgccgaggcactatctagggaacctggtgacaactgtctgggcttcccatcaccgtccggctgagaaggacccggagaccatggtatccccagtaccggcgtgcaaccacccggagatagacagagacgaccttctgtggattgctcacgtggcttttcgtattagattggggctgaacgcggttaatgaagagcacattaggggtttgctgcattacctacattctcaggatgattgggagcagatcgggatacattttacggaccccatcttcatctcctcctggcgtcatctgaaggtgtatgagctggacttcggcccgggcattggccacgtggaaaactttgaaatggacgtagggacaactgacggagtttgtgttgtgatgcctgctaataaaagagcagtggggaaaacggagaaagcaccctgggacatccgtatagtcttgaattccgatgtgatggaagcactaatcacagatactctattcggatggcccatggtaagagacgcatctaag

SEQ ID NO: 98 - DNA, 1047 bp, synthetic construct
  source 1..1047: mol_type = other DNA
atgtcatccggctataatcatagcgtatttgacgggaccggcgcgggaaacattctagagatgcttgcagattgctgcagagccgacccggcgtctatcattagcctacctacgactgagtcaatagagtcagagttacgtgagctattgagtgacccaggggttgccgtcgcaaaccattcacaggcggcctatgcaatcaactgcgcacacacggaagtagagcccgaatctttccctgcgatgttgtgctcatggccatttttgttatcttctgaaaagatagagtgtctgagagaagcatgtaactctttgctgccccatttggtaaggacttacagggattctcagtcttccatgatttaccctgaccctaactggccacaaattttgtctagcaatgacgtattgacggcgttactagcggtgtcagtggaaaaagcgagggaagctacaggagcacaggagcacatgagctcttctctagcgatggccgttaatttgagggagagactaaaaccaatgcctcgtcattacttaggaaaccttgttacgaccgtatgggcatcacaccgtagacccgctgaaaaggacccggagacgatggtcagtccagtacctgtctgtaaccaccccgagatagacagagacgacttgttgtggatagcccatgtggcatttaggatcaggctagggttaaacgcaattaacgaggagcatattaggggcctgctacattatcttcacagccaagatgactgggaacaaatcggtatccactttacggatcccattttcataagttcttggaggcacctgaaggtgtatgaacttgacttcggaccgggaatcggtaatgtcgagaactttgaaatggatattggtacaacggacggcgtatgcgtcgtaatgcctgcaaataagcgtgctgttgggaaaacgaagaaagcaccctgggacatacgtatcgtgttaaattccggggtaatggaagcattgattacagatactttgttcggctgggcaatggtaagagatgcttccaaa

SEQ ID NO: 99 - DNA, 957 bp, synthetic construct
  source 1..957: mol_type = other DNA
atggcagccggttataatcatagtgtattcgatggaactggcgcaggcgcgatcctagagatgctagccgactgctgtagggcaggcgcggcttcagaaactagtcttaccaccgcaggggacatagagtcagaaccacgtgggttcctatctagtgccagcgttgccgtaccgaaagatagccaagctgagtacgttattaactgcgcccatacagaggtagaggctgactcatttcccgcaatgctatgtaactatccgtttttgttgccaagtgaaaaaatcgagagattgagggacgcgtgcaactcattacttccatacttggtggacacatatagtcgttcccagaagagcagagtcaacgacgacgcaaaatggcctgggttcctgtccagcaacgacgtactaactgcgttgttagcgattgaagttgagagagccagggaagctgcaccatctccccaaccgaaaagcacgtcccttgctatggctgtaaacctgcgtaaaagacttactcttgtgccttcccactacctgggaaacctggtcacgacagtttggactccccacttacaaccgaccaagcgttgggataccttgttaagcacatcacccgcatgtgaacaacttgaaatagaaatgtctgaccttttatgggtcgcggacgttgcattcagggtcagattaggactatcctctatcgacgaggagcacgcttgtggcctaatccactatcttcattctcaggacgattgggagcaaattggaatacactttactgaccctgtgttcattagttcatggcgtcatttgaaggtttatgagcctgactttagcccaggtattggccagattcaggacttcggaatggaagtaggcacttcagatggggctatggacgcactgatgaatgaccccctttttggctgggccagggtgatagatacgttcgaa

SEQ ID NO: 100 - DNA, 1314 bp, synthetic construct
  source 1..1314: mol_type = other DNA
atgtcatccacaaaaacagatgcgcaggggaggcatcctcaagaaacggccactcatgcggctactacggatgaggaggtacaggaccaatggagggcaccgttcgaggtgcttagtagaacattggtctttcagcatgaggatcacagattatggtgggagcgtgcggcctcaaagttagcgacatacttacgtctggcgaagtacagtgtggggtcccagtatcagcacttactaatgttttatagcgtgtatgcaccaaacctgggcccgtggccaaatgataaaagagataacgtacattgggtctgtggtatatgccctggtggggaaaatcttgaaatttccatgaattatcagcagggtgccaaatgcactgtgagaatagcagcggaaacgatcacgccggcggcggggacggataaggacccgttcaatctgaccgctgagaagaagatgatcgaggatctaaaagcactgcaaccgaatctgaatttcacctggttcaatcacttccaaagggaagtcctagtgcccgaagaagtcgcgctaaacaacgatgagattatatcaaaggtcccgtttaagaaccagcgtcttcacgggctggacttatctgagggggcgtttatgcttaagagctactttatgcctgctattaggagtgcgattactggggttgaaaatacacagataatgttcgaatcaataagaaaacttaatctaaaaaacgcaaatttcatatcagctctgtctaccctagaagattggatggtacctacgaatggcagattcatggagtactgggatggcatctcttacgatgcggtcgatgcgtgcaaggccagaatcaagatctacaccggcattagaatgaaatctatcgagcacgcgagagatgtgtggactctgggaggtcgtttgcaaggagaggacatagaaaaaggatttgaccttgtggcaagattatggagacgtttaatggacgaagagccctctacgtgcgagatgaaatactggatgcagtgggtttgggaattaagaaccgatgtaccatttcctgtacccaagctgtacttcagcgtggcagcagcagaagaccactatgtcagcgacactgtcgttgaaattttagactacctaggttgggatgatctggtccagacacatagagccctgatggacgaggcctggtccttaggccagactactaaaagctatctggctttttcctacatcagtgttacctttcacagcatcaagggcccttacataacaacgtatggcaatccgtccgggcccagaccggttttt

SEQ ID NO: 101 - DNA, 1194 bp, synthetic construct
  source 1..1194: mol_type = other DNA
atggcagccaccaagtcagatgctcaggggagacatcctcaggagactgccattcatgctgcgactaaagacgaggtagtccaggatcagtggagggccccttttgaagtcttgtctaggactcttgtgtttcaacacgaaggccacaaactgtggtgggaacgtgccgccagtaagctggctacctatttgaggctggcgaagtatagtctggtatctcagtatcaacatcttctaatgttttacagtgtgtacgcacccaatctagggccttggccaaatgaaaaacgtgacaacgtccactgggtctgtggcatttgcccaggaggcgagaatttggagctatcaatcaattatcaacaaggctccaaatgtactgtccgtattgccgccgaaaccattactccggctgccggtaccgacaaagacccgtttaatctaacagcggaaaaaaaaatgatcgaagatttgagagcgatacagccagaccttgattttacatggttcgaccattttcaaagggaagtgcttgtaccagaggaggttgtgttatcaaatgatgacgtgcttagtaaagtacccttcaaaaaccaacgtctgcatggattagaccttagcgaaggtgcctttatgttaaaaagctacttcatgcccgcaattcgttccgcaattacgggtatcgaaacgacgcaaataatgtttgagtctatccgtaaattaaacttaaaaaatgctaatattatcagtgcgttaaacacgcttgaggactggatggtgcccactaatggatgctttatggaatactgggacgctgtctcatatgatgccgtcgacgcggacaaagctaggatcaaggtctacactggaatatgcatgaagagcttggagcaggccagggatgtgtggacgctgggcgggaggctacaagatgaagatattgaaaaaggtttcgatctggtagctagactatggcgtaggttaatggatgaggagccctccacctgcgagatgaagtattggatgcaatgggtttgggagttaaggacagacgtgcctatccccgttccgaagttgtacttttccgtcgcagcagcagaggaccactacgtaagcgacactgttgtggagattttagactatttggggtgggatgagcacgtacagacacacagagctcttatggatgaagcgtgg

SEQ ID NO: 102 - DNA, 1194 bp, synthetic construct
  source 1..1194: mol_type = other DNA
atggcagccactaagagcgatgcccaaggcaggcacccgcagcaaaccgctatccacgcggcagctaaggatgaggtagtacaggatcagtggcgtgctcctttcgaagtcctgtctcgtacccttgttttccagttagaagaccacaaactgtggtgggaactagcagcgagtaaactggcaacctatcttaggcttgccaaatactctgttgctagtcaatatcagcatctgcttatgttttactcagtgtacgctccgaacctgggcccatggcccaatgaaaaaagggataatgttaggtgggtgtgtggtatatgtccagggggcgaaaacctggagctttccatgaattatcaacaggggagtaaatgcacagtaaggatagctgccgagacgatcacgcccgcggcggggactgacaaggacccatttaatctgacggccgagaagaaaatgatagaagacttgcaggccatccaaccgaatcttgattttacttggttcgatcacttccagcgtgaggttttggtacccgaggaggtagtgctgagtaacgatgatgtaatttccaaagttccatttaagaatcaacgtttgcatgggttggatttaagcgaaggagcgtttatgctaaaaagctattttatccccgctatccgtagcgccatcaccggaatagagacgacgcagattatgttcgagagcatcaggaggcttaatttgaaaaatgcaaacattgtcagcgccctgaacacgttagaagattggatggtaccgacaaacggccgttttatggagtattgggatggcgtctcttatgacgcgatagatgcagacaaagctaggattaaagtgtatacaggaatttgcatgaagacgttagaacaagtccgtgacgtttggacgctaggtggcaggctacagggaggggatatcgagaagggatttgatctggtcgccaggttgtggcgtagactaatggatgaagaaccgtctacctgcgagatgaaatattggatgcagtgggtgtgggaactacgtacagacgtgccgatacctgttcccaagctttacttcagcgtcgcagctgctgaagaccattatgtgtctgatgcagtcattgagatattagactatcttggctgggatgagcatgttcagacacatcgtgcactaatggacgaggcttgg

SEQ ID NO: 103 - DNA, 1278 bp, synthetic construct
  source 1..1278: mol_type = other DNA
atggcagccaccaaagacacgactcaagtctctcactctcaagaagaattgcgtgctccgtttgaggtgttatctagaaccttgagttttgaacacgaagatcatagactgtggtgggagcaaggtgggtcaaagttggcaacttatctaaagcttgcgagatattcaatggcgtcccagtaccagcatttgctgatgttttactcagtttatgccccaaatctgggtccgtaccctaacgagaagcgtgataatgtgaattgggtctgcgccatttgtccgggtggtgagaacctagagctgtccatgaattttcaacagaggtccaagtgcactgtcaggatggcagcggaaacgttggggccgctaaccggaactgagaaagatcccacaaatgttgccgctgaaaacaagctgatcgatgacctgagggctcttcagcccgaccttaatttcacgtggttcgggcactttgagagagaggtcgtcgtgcctaaagaagtggcactaaatcataaacaagcgataaattctgtcccgtataagaatcaaagacttcatggccttgatctagcggaaggggcgttcactttgaaaagttacttcttgccttcaattaggagtgccgtcacgggggtagacaccacgcaaattatgttcgattccattaggaaacttgcgttgggtaaccctaattttaactcagccctggacatgttgcaagattggatgattcccactagaggaagattcatgggcgattgggatgtggtatcatacgatgcaatcgacgcggaggaggcgcgtattaaggtttatacaggcatcaagatggaatcactagagcaggcgtgggatatatggacattgggcggaaggctacagggcgacgttgtcgataaaggattcaaattggtaaagagactgtggggaagactaatggatgacgagccgtccacctgtgagctaaagtattgcatgcagtgggtgtgggaattgcgtactgatgtttcaatccctgtacctaaactttactttacggtagcagaagcggaggaccactatgtcagcgacgctgtcgtcgaaatcctggaatatttgggctgggacgaacatgtgcaaacgcaccgtgccctgatggacgaggcttggagcttaaaccaaaccaaggaatcttattttgcttttaactatatttccgtcacataccactctataaagggcccgtatataactacttatggcaatccaagtgggccaaggccggtgtttaaagaa

SEQ ID NO: 104 - DNA, 1185 bp, synthetic construct
  source 1..1185: mol_type = other DNA
atggcagccactaagggggacgttcaggtattccacagcgaggagaaattgagagatcctttcgaagttctttctttgaccttatcctttcaacatgaagatcatagactttggtgggagcagggggggtttataaggagaatttaccttagactggccaaatatagcgttgcctctcagtatcagcacttgttgaccttttactccgtatatgcgtcaaatcttgggccttggcctaacgagaagtcagacaatgtcaactgggtttgtgggatttctccaggcggaaaaaacttggagttgagtatgaattatcaacagcaatccaaatgcacagttaggatcgcggctgagaccatcgggccactaacggggactgagaaagacccttccaacatgatcgcagaaaagaaacttattgaggatctgcgtgcgctacaacccgatcttaatttcaattgtcacaagaaagccattgacaccgtcccgtttaagaaccagaggttgcacggtttggacctagcggagggtgcctttatgttgaaaagctactttatgccctcaatcaggtctgctgtgacgggagtcgaaacgacgcaaatcatgtttgacagtatcaggaagcttgggcttgagaacaacaacttcaactctgcgctggatatgctacaggactggatgattccaacaaatggacgttctatggaacactgggatggagtctcttacgacgcgatagacgcggagaaggcgagaattaaggtatacactgtgattaacatgcaatcattagagttagcgtgcgaaatttggacattgggaggaagacttcaaggcgatgtcgttgataaaggatttgaacttcctagcacttgcgagttaaagtattgcatgcaatgggtatgggagcttcgtacagatgtacccataccggtgccaaaactatactttactgtggccgaagcggaagatcattatgtgagcgacgcagtagtcgaaattttagaatacctaggatgggacgagcaagtgcaaacacatagggcgctgatggacgaggcctggagtctgaatcaggcaaagaaaagttatttcgcattcaactatatttctgtaacctaccacaatatcaaaggaccctacataactacgcatggtaaccctagcggccccagaccggttttt

SEQ ID NO: 105 - DNA, 1083 bp, synthetic construct
  source 1..1083: mol_type = other DNA
atggcagccttctattcagtatacgctcctaacctaggaccgtggcctaatgagaaaagagataacgttaactggatttgcggcatttcccctcgtggggagaatctagaactaagtataaactatcagaggggatctaagagcacggtccgtatggcagcagaaacaataggaggcttggcaggtaccgagaaagaccccagcaacgtaattgccgaaaagaaactgatcgagggtctgagggcgctacagcccgaccttaactttacgtggttcgaccacttcgagcgtgaggtcgtagtaccggaagaagtggcgctgtcccacaaaaaagtgattgacaccgtacccttcaaaaaccaacgtttccacgggctagacttaatcgagggtgcgtttatgttaaagtcctatttcgtgcccttgatccgttccgctatcactggtgtcgaaactacgcagatcatgtttgactccataagaaaacttgctttaaaaaatccgaacttcaattcagcgttagacatgctgcaggattggatgatccctaccaatggtagattcatggagcattgcgacggtgtatcttatgacgccattgacgcggaaaaagctagaattaagatctacagtggcatcaagatggaatctttggagcaagcacgtgaaatttggaccctaggcggcaggctaagaggggacatcgtggataaaggctttgagttagtaaagcgtctgtggcgtaggttaattgatgaggagccctctacctgtgagctaaaacattgtatgcaatgggtatgggaactgagaacggatgtctcaattccggtccctaaggtgtactttactgtatctgaagcagaagaccatacggtatctgacgccgttgtcgaaattttagaatacctaggatgggacgaacatgtccaaactcatagagcccttatggacgaggcatggtctttaaatcagactaaaacaagtttcttcgcctttaattatatctctgtgacgtaccataacattaaaggcccatacataacaacttatggaaacccatctggtcccaggccggttttc

SEQ ID NO: 106 - DNA, 948 bp, synthetic construct
  source 1..948: mol_type = other DNA
atggcagccacctcccaacaccaagaacatacggggactaaacgtttttcaattcaatccgaccccgtggagatacatagagcaatcgttgaggacggtgtggcgattattgaggggtttttaacgccagaacaagttcagaaacttaataaggacgtggatgccccgttaaaagcagaccgtgaacagttgaaatttaaggctgataagaaggatgatccacatttctggcttgcagatttcattcccgatcatgtggcccgtgtccacaaccttgtagacttcagccattgtttccgtcatgagatcctgaaccacgaacttttgcacaaaatctgccgtttaacattcgaggagagtggcgactactggctgggttacggcgcagttatagaaaacgggccggggaccacggagcagaaatggcacagggaccaaccgcgttatccattagtaaaagaagggcctgacgcaccagaggggatgttaaattttttcacggctttgacagacttcgacgctgaaacgggtaaaacccagtatattttaggtagtaacaaacgtgtggagttgggtgagccagacgccgaccaccctatcgaatacgtagggctaaagcccggtgacactacaatagtgtcaggaaagataactcatagaggctcagataatagaagtgataagatgaggagagccatgccgataatgataattccgtctattttgacgccctttgacgcaacatgccatttgtccagagaattagttgaaactatgacgcctctggcgcaaaaaatgatatgtcgtcgttcagttatgattccggcacctggcactgtagaagtgaaaactggcatatggtgcgtaaatatgagagaggcgggcgaacaaataggcctaaaatcaaatcaacgtgcgaaagaggatgccgaggcgacggatgccgta

SEQ ID NO: 107 - DNA, 948 bp, synthetic construct
  source 1..948: mol_type = other DNA
atggcagccacatcacaacatcaagagcatgctagaacaaaaaggttctcaattgaaagtgatcccgcagaaatacatcgtgcaatagtcgaagatggtgttgccattatagaggggttcctgaccccagaacaggtacaaaagttgaacaaagacgtagatgccccactaaaagctgatagagaccagttaaagttgaaatcagacaagaaggatgatcctcacttctggttagcagactttattcctgaccatgtagcccgtgtccataacttggtggacttttctcactgctttaggcacgagatcttaaatcacgaattgctacataagatttgcagactgactttccaggaatcaggagattattggttgggctacggcgcggtaatagaaaatgggcctggcacaactgaacaaaaatggcatagagatcaaccaaggtaccctttagtgaaggagggacctgacgcacctgagggtatgttgaacttctttactgctctgacagacttcgacgctgaaaccgggaaaacgcagtatattctgggctcaaacaagtgggtcgagctgggtgagcccgatgcagaccatcctatcgaatatgtgggactgaagcctggcgacaccaccatcgtcagtggaaaaatcacccatagaggcagtgacaatttctccgacaagatgcgtagagcaatgccaataatgattataccctccattttaacgccgtttgatgccacttgtcaccttagccgtgaattagtagaaaccatgacaccgcttgcccagaaaatgattggaagaagaagcattatgatcccggcaccgggcaccatagaggtgaagacgggtatttggtgtgtgaacatgagggaggctggcgaacagatcggattgaaatcaaaccaattggtcaaagaaaatgcggagaccactgacgccatg

SEQ ID NO: 108 - DNA, 948 bp, synthetic construct
  source 1..948: mol_type = other DNA
atggcagccacaagtcaacaccaagaacatgccgggactaaaaggttctcaatccagagtgatcccgccgagatacatcgtgccatcgtggaggatggcgttgcaataattgagggatttctaactcctgaacaggtacaaaaattgaataaagacgtggatgccccactaaaggcggacagggaacagtcaaggctaaaaccagacaagaccaacgtaccgaatttttggatggctgattttgtacccgaccatgtcgctagagtccataatctagtggacttctcacattgcttcagacatgagatattgaatcacgagttgttacataaggtttgtcgtcttacgttccaagagtctggagattactggcttggttacgctgctgtgatcgaaaatggtccgggtacttcagaacagaagtggcacagagatcagccaagatacccccttgtcaaagagggcccagatgcccctgaaggcatgttgaatttttttacggctctgacagactttgactctgagacaggtaagacacagtacatcttgggatcaaacaaaagggtcgagttgggagagcccgatgcggatcacccgattgaatacgttggactgaaacctggggatacaactatagtctccggaaagataacccatcgtgggtcagacaatagatcagaaaaaatgcgtagagcaatgcctatcatgatcatccctagcattctaacaccattcgacgctacatgtcatttatcaagggaattggtggaaacgatgaccccgttggcgcagaaaatgatagggcgtaggtctgtgatgattcctgcaccgggcaccatcgaaatcaaaaccggaatctggtgcgtcaatatgcgtgaggcgggggaacagatcgggttgaagtcaaaccagagagtcaaagaagacgccgaggcaactgatgcagtg

SEQ ID NO: 109 - DNA, 957 bp, synthetic construct
  source 1..957: mol_type = other DNA
atgtcatccacttctcaacaccaggaacatgcaagaacgaagagattcagtatacaaagcgacccagcagaaatccatagagcgatcgtggaggatggcgtagcgattgtggaggggttccttacacctgaacaggtacaaaaattgaataaagatgctgatgcaccattaaaggcagacagagagcagctaaagctaaagccagacaagaaggatgacacacatttctggctggcggattttatcccggatcacgtagcgagggttcacaacttggttgattttagctactgcttcagacatgagatattaaatcacgagcttttgcacaaaatatgcagacttacatttcaagagtcaggcgattactggctggggtatggagctgttatcgaaaatgggccaggaactacagaacagaagtggcatagggatcagccacgttatccactggttaaagaaggcccggacgctcctgaagggatgcttaacttctttactgctttgacagatttcgatgcggaaaccgggaagactcaatacattttgggttcaaacaagcgtgtcgaattaggtgaacccgacgcggatcaccctatcgaatacgtggggctgaaagcgggagatactactatagttagcggcaagatcacccatagaggatctgacaacagatctgataagatgaggagagctatgccaatcatgattataccaagcatccttacgccatttgacgcgacttgccacctgagcagagaactagttgaaactatgacgccgctggctcagaagatgattggtcgtcgtagtatgatgataccagcgcctggcacgattgaagtcaagactggaatttggtgcgtaaacatgcgtgaggcaggtgaacaaattggattgaagagtaatcagttagtcaaagaggatgccgaggcatcccaggccaccgacgctgca

SEQ ID NO: 110 - DNA, 948 bp, synthetic construct
  source 1..948: mol_type = other DNA
atgtcatccacttcccagcaacaggaacatgcgaggactaagaggttctcaatcgaatccgatcctgcagagattcaccgtgcaattgttgaagacggagttgctatcatagagggttttttaacgcccgaacaagtgcaaaaacttaataaggatgtcgatgctcccctaaaggctgatagagaccaattgaaattgaaaagcgacaagaaagacgacccacatttttggttggcagatttcatacccgatcacgtagccagagtgcataatctggtagacttttcccactgcttcaggcatgaaatattaaaccacgagttacttcacaagatatgtcgtcttacctttcaagaaagcggcgactactggttgggttatggggcggtgatcgaaaatggtccgggaactatagaacagaagtggcacagagatcagccccgttaccccttagtcaaagaagggcccgatgcaccagaggggatgcttaacttcttcacagctctgacagacttcgacgcggagacaggaaagacccaatacatactgggtagtaataagtgggtcgaattgggcgaacccgacgctgaccaccctatagagtacgtgggcttaaaacctggagatacaactatagtgtctggtaaaatcacccataggggttcagacaactttagcgacaaaatgcgtcgtgcgatgccaataatgataataccgtcaattttaacgccgtttgatgccacttgccatttaagcagagagctagtcgagactatgacaccgttggcacaaaagatgattgggaggagaagcattatgattcctgcgcctgggacaattgaagtcaagaccggaatctggtgcgtaaatatgagggaagccggagagcaaatcggattgaaaagtaaccagcttgttaaagaagatgccgagacgacggatgcggtc

SEQ ID NO: 111 - DNA, 918 bp, synthetic construct
  source 1..918: mol_type = other DNA
atgtcatccgagagtccgccccagcagcgtactaagagattttctatggactccgatccggtcgcgattcaccaggcaatcgtggaagacggtgttgcagtcatagagggtttcctgacccccgaacaagttgaaaaactaaacagagatgtagatgcgcgtcttttagcttatcgtaatagagcgagacctgaggtgacagacaagcaaaagctgtggaccgctgatttcgtgctagaacacgttgcgagagttcacaatctagtagatttctctcacgtctttcgtcatgagatattgaatcacgggctactacacgaaatttgccgtaggactttctgggagagcggtgattattggctagggtttggcgcggtgattgaaaatggaccgggcacagaacaacaaaaatggcacagggaccaacccatctaccctctagtcaagcaggggccagacgcgcccgaaggaatgcttaacttctttactgcactgacggacttagatgatgagacgggccgtactcagtatatatggggctccaacaaatgggaagagctgggaaccccggacgctctacacccgatagagaatgtggccctaaaagctggagacactaccatcgtgtctggtaaggttactcatcgtggctccgctaatctgtcaaggaaattcaggagagccatgcccataatgatggtcccgtcaatcttgaccccttttgatgccacctgccacttgtcccgtgaacttgtcgaaaccatgactccccttgcccagaagatggttggtaggcgttcagtaatgatcccacctccgggcactacggagggtgtcagaactgggatctggtgtttgaacatgagggagcttggtgagcaacttggtctgaagtctaaccaacctgaacaaattgtc

SEQ ID NO: 112 - DNA, 930 bp, synthetic construct
  source 1..930: mol_type = other DNA
atgtcatccgcagcattatctcacccacagccccagattcagcgttttccagtaaccagtgacggcgcggcggtctacaaagcgctggctgatgacggagtcgtgattatagagggtttcttatcaccagagcaagtcacaaaattaaaccgtgatgtagatgcaccgttaatctctatgagagaagatgggaaaatctctaagccgccgaaggaaggccagtccacatattggatggcggatattgtccctcaatacgttcaaagagttcacaaccttgtcggcttttcaaaagtcttcaggcacgaaatactgaaccacgagcttatgcacgaactatgtagattggccttcgaagtaagtggcgattactggttgggatatggtgcagtgatggagaacggccccgggacgcatgagcaatcatggcatagggaccaacccaattaccctcttctgcgttctggccctggaacagcggaaggaatgttaaatttctttaccgccctaactgactttacccctgaaaacggaaggacacagtacatctggggttcacataaacttgtggaaataggcgaacccgacgccgaacacccggttgtctttagcgatctaaaagctggagacaccgcggtgctgtctggaaagattgttcacaggggaagccttaatgccactttggatacctacaggagggccctagcccttattattataccaggtgtactgactcccttcgacgcaacatgtcaattaccaaggagggtcgtcgagaccatgactccactggctcaaagaatggtaggaaggaggagcgtccgtgtcagcccgccttccgctataggcatctggtgtttgaacatgcgtgaagttggagagcaaatgggtcttaagagcaatcaaccagacgaggagggagaggaagaa

SEQ ID NO: 113 - DNA, 927 bp, synthetic construct
  source 1..927: mol_type = other DNA
atgtcatccaccgtgcaatctaagcctcaagctcaagtacagagattcagcctaacgagtgatgcagcagccgttcacaaggcgcttgcggacgatggtgttgcgattattaaaggctttttaagcccagaacaagtcgctaaactgaacgccgacgtggatccgcctctatttgccttacgtcaagctgaaaaaccggctaacccgtctgccgcgagcgtctggatggctgatttacaccctgcccatgttcaaagagtccacaatcttgtcggtttctcaaaggttttcaggcacgacattcttaatcacgaacttatgcatgacgtatgtaggcttgcgttccaagagtctggtgattattggttgggatacggagctgccatcgaaaacggccctggcacacaggagcaaatttggcatagagatcagcctgattatgccttactaaaagcaggccctggtactgcagaggcaatgctaaatttcttcacggccttgacggattttacccccgaaacaggtatgacgcaatacatatggggctcacataagagagtggagctaggagaacccgatgccgaacatcctgtagtgttcacgaaattaaaagcgggggatactgcagtgttgtctgggaagatagttcacagggggagcgccaatgcaactccggacgtctttaggagagctctagccttgatgatcattccagctattatgacgcccttcgatgctacctgtcatctgtcacgtcacatggttgagacgatgactccattagctcagaaaatggtaggacgtagaagtgtggtaataccgcctccgcacacggtaggcgctgctttaggaatctggtgcctaaacatgcgtgaagttggtgagcagatgggtctgaagagtaaccaacctgataaggaagaggag

SEQ ID NO: 114 - DNA, 930 bp, synthetic construct
  source 1..930: mol_type = other DNA
atgtcatccaccacatcttcacatacgcaacctaacatccagagattcccagctacttcagacggggcggccgtgtacaaagctttagccgacgatggtgtcgtcattatagaaggattcttgtctcaagagcaggttgagaaacttaacgctgatgtcgatcatcctcttgtaagtcttagagaaagcggaaaattagagaaaacctttaaagaagggcaatctacgttctggatgaccgagctggtcccccagtacgtgcagagagtgcataacttagtagacttctctagagtatttaggcatgacgtattaaaccatgagttgcttcaccagctttgtacgttggcgtttgagccctccggggattactggttagggtatagtgctgtaatagaaaacggacctggaaccaaagagcaattttggcatagagaccaacctaactacccgttgttacgtagcggaccaggtactgctgaaggaatgctgaatttttttactgccctaacggatttcactccagaaaatgggagaacgcaatatatttggaattcccataaacgtgtggaaattggagaacctgatgctgaacatccggtactgttctcagaccttaaagctggggatacagctgtactaagcggcaaaatggtacatagagggtccgcgaatataacgtccgacttttatagaagggcattagctacgatgataatacctggggtattaacaccattcgacgcaaactgccaaataccgcgtcgtgtcgtagaaactatgacccctcttgctcaacgtatgattggccgtaggtcagttagagtctctcctccttctgctgtgggtatttggtgtcttaacatgaaggaggttggcgaagagatgggcttgaaatctaatcaaccggacgtggaaggtgaaaacgaa

SEQ ID NO: 115 - DNA, 1629 bp, synthetic construct
  source 1..1629: mol_type = other DNA
atgtcatccccatcagaccagaaaactaggtgcatagtgatcggtgcgggggtctccggaatacttatggcctataaattacaacactatctaaatgattttgtggaatttcagattttagaaaagaaccctgatttaggtggcacatggtatgaaaataaatatcccggctgtgcttgtgatgtccccagccattgctatcagtacgcctttgcgccaaatcctagttggagtaagctgtatagtagttccgaggaaatccacgactatctgaagaaggttgtgaagcattttaatctagaaagatacatccgttataatacgaaagtcactagtgcgaaatggaccgagagcacggggacctggagcgtaggcatatctgatggaacgacaatagattgtgagatcctgatcaacgccggtggtatccttcatcatccacagttgccagacttaaaaggattgaagtcctttgctggaaaagttgtacatacggccgcatgggattctgggttggatttagggggaaagaaagtagcattaattgggtctggttcttccgctgttcaaatactgccccaattacagcctgtctgctccagcgttagggtttatataagaacgcctccctggattgcccccccgatggttgatactgagtctgaaagtccaaaccgtgattacacgaacgacgaacaagcgttttttcgtcaggaccctgctaagtacgcacaaatgagaaaggagattaactctaatttgaatgggatgtttaaagcattgtttaagaactcccccgagcagcaaaactttagacagatgctagagaccagaatgaggagtctaattagggatgagtctttacaggaccaccttgtccctaaattcgaggttgggtgcagaagggtaaacccatgcgaacaatttctgtattctatacaagagaagaatgtcacagccgtctttaatgccattgaagaggtcaaaccgcacggaatagtttctgaaggggtcgaacatcccgtagacataatagtagcagcaaccggcttcaataactcatttcgtcctcaattcccgcttttggggcgtgacggggtgaatctgcaggacatctgggctgatgagcccacctcttattttggcacgggggttagtgggtttcccaattaccttaccttccttggcccgaatacgcccatcagcaatggtgcgttcatgggcccacttgaagggacttcagactattttattaggttattgcagcgtgccatcagatacggtgcaagtagctttgacgtcaagccagaagcgcaggcagattttgatagccgtgtgtacaaaaagacaagagacgggaagataactgctctttggccggggtcctctctacagtacatgcaggtgcttgctgaagatagatgggaggactacaactgggcatacagagggaggagatacgattactgggggaatggcttctcatggattgagcaaccaaagtctgacaaattagggattcaggagcgtaagtcaaaaaacgagatggtcactattcctaatatcgacgcagacctggggttttacataacgcaacagaccccgttaccggaggccgatactgaagac

SEQ ID NO: 116 - DNA, 1668 bp, synthetic construct
  source 1..1668: mol_type = other DNA
atgtcatccgacgcgagccagcagaaacttcgttgcgtcattatcggggcaggggtgtccggtatattgatggcttacaaattgcagaagcatcttcgtgagtttgttgatttccaagttcttgagaagaatccggagctagggggcacttggtatgaaaataggtatcccgggtgcgcgtgtgatgtgccttcccattgctaccagtacagtttcgcccccaaccctagttggtccaaaatatactccccgtcagaagaaatacatgcttatctaaagaaagtggcaagtcattttaacctggagagatacataagatatcgtaccgaagtagtctccgcaaactgggacgaggttaccagttcttggaacgtcggaatcaatgacggaacgattatggcctgcgatgtcctgataaatgccggcgggatcctatatgaccctcagtggcctagtatagagggtctgggaaggtttaatggaaaactgatacatacagccgcgtggaataagaaactagacctgaaggctaagagagttgcactaatagggagcggtagtagcgcagtacaaatcttacctcagttacagcctatatgctcctctataagggtctatattaggacgccaccctggatagcaccgccgatgatcgaaacagaccttgaatcaccaaaccgtgattacacctctgacgaaaaagcgtcatttagacagaacccggaaaaatatgctgaaatgagaaacggtataaacagccaatttaacggcatgtttagggccttgttcaaaactagcccagaacagcactacctaaggcagatgctagaggctagaatgaggagtcttatccgtaatgaggaactgcaagagcacctgattccgcgtttcgaggtcggttgtagaagggttaatccatgcgaacatttcctgaggagcatacaggagaaaaacgtcacggctgtgttttccgcgatcgaggaagtgacccagaacgggattgtctccgaaggcgtagagcaccccgtggatactattgtggcagctactggattcgataatagctttcgtccaaaatttcctttaagaggccgttatggagtgaacctacgtgacttgtgggctaacgacccaatttcctattttggtacgggagttgccggatttccaaactatttaacttttctaggaccaaatactccgattgcaaacggagccttcatgggaccactagaggggacctcagattattttatacgtctactgcaacgtgcgataaggttcggggcctctagtttcgatgtaaaggcggaggcacaggccgactttgattccagaattcacaagtttatggaaaacatggcctggaccggcagttgcagaagttggtacaaaagtggcagggacggaaaaataactgccttgtggccgggatcaagtctacactatatgcaagttctggcagaggacaggtgggaggactacaactgggtctataagaggccccgttatgactactggtgtaatgggttgtcttggattgaagatcccgaatcagaccgtttgggttttcaaatgcagaaaagcagggaagcaatgacaactatcgccagtaccgacgctgacttggggttctatattaagcaagaaacaccattgccagaaata

SEQ ID NO: 117 - DNA, 1671 bp, synthetic construct
  source 1..1671: mol_type = other DNA
atgtcatccgatacctcccagcagaagagaaggtgcgtgatcataggagcgggggttagcgggatactgatggcctgtaaactggagacgcacctaggagagttcgtagacttccatgtgttggaaaagaatccggagctagggggaacatggtacgagaacagataccctggctgcgcctgtgatgtaccctcacattcatatcaatacagtttcgcccctaacccgagttggagtaaaatatatagtcctagtgaagagatccaggcctatctgaaaaaggtcgtttcacatttcgggttagagaagaggatccaatatggagtggaagtcgtgtctgccgattgggatgaagcgtcttcaacctgggccgtggggctgaagcatgggacaaccatgaagtgcgacgtattgatcaacgcgggcgggatactacataagcctcgttggcccgaaattgagggcctagatcgtttcaagggagcattagttcacaccgctgcttgggataagactctagatttggccgggaagactgtaggtctagtggggtctggatccagcgcaattcaggtactaccacatctacaaccgatatgctcttccataaggatgtatatacgtaccccggcgtggatcgctcccagcaccatggagaagaaagaagaccccggaccgagtaatagagactatacgtccgacgaaaaggcgtcattccgtcaggaccctgagtcctacgcggaaatgagaagcgctatcgacgcacagtacaatggtatattcccagcactgtttaagagttctcccgagcaaagacacttgcgtcaagtgttagaagcgagaatgcgtagcctggtccgtgacgaagccttgcgtgatagattggtccctaggtttgaggtggggtgcggcagagtaaatccgcacgaagactttttgaggggaatacaagaggaaaacgtgactccagttttcgatgctatagaagcagttacacaaaatgggattgtctctggcggtgttgaacacccagtagacgccatcgtagcagctacgggatttgacaacagtttccgtccacgtttccccctacgtggaagacacggagttgatttagcggacctttgggcagaagatccagtatcatacttcggaacagctgtggcgggtttccccaattacttaacctttttgggtccaaatactcatatagcaaacggggcatttatgggccccttagagtccacaagtgattatttcataaggcttttacagagggctataaggtttggagcttctacattcgatgtgaggccagaggcccaggcagatttcgataggaggattcacagctttatgggcaacatggcctggacgggtagctgtcgttcatggtataagtctagaagatccggtaaggtaactgcgctgtggcccggcagcagtttacactatatgcaagtcctagccgaggaccgttgggaggattacgattggacttacaggagaccgaggtacgaatattggagtaaagggttatcatggattgaagatccggaaggagataggcttggagtacaaatccagcgttcacgtgaaaccatgacaaccattgctggcacggacgcgcacttagcgttttatatcaagcagacagcacctctgcctgag

SEQ ID NO: 118 - DNA, 1734 bp, synthetic construct
  source 1..1734: mol_type = other DNA
atgtcatccactttgagcgagtgtagaaatagaccgattcatgcgaaccgtctgatgagggttatatgcataggctcaggggctagcggtctgtacctagcgtacaggttgcagtcatgcttcacggattacacgctagatgtttatgagaagaacgcagacgtaggaggtacctggttcgagaacagatatcctggttgtgcatgtgatataccagcccacaactatacttacacatttgagccgaacgcaaattggtcagccaattatgcatcctccagtgagatcgccaggtatttcagcggtttcgccgacaaatatggactgaggagatatattaaattcgaacacgaggtaattggggcgagatgggatgaagccagaagcgaatggagtgtacgtgttcgtaaaacagatgggaaagtgttcgagcacgcatgtgatttcctgatcaatgcggctgggattttgaatgcctggaagttgcctgacatcaccggattagactcttttaagggaaccttggtgcatacagcacgttggaatgaagctattgatgtggcagggaaaaccgtggggctagttgggaatggtagctctgggattcaaatactgcctgagttacaacgtactgcccagcacgttacagcgtttctaagggggccgacctgggtatcaccggcgaggggtatggaataccatatctacacggaggaagagcgtaagaggttccaggacacgccaaccgagttgttggccatgagaaagcaaaccgaatccttcatgacgtctgcaactttgttcagcttattcattaaagggagtaaagcccaggaggtattagcgcaagacatggaggaccatatgagaatgaaaatagacaatgaccttttagcacaggccttaattcctgaatacccgcttgggtgcagacgtccgacaccagggatcaattatctggagtctctagcgaagccaaacgtcagggcgtaccatggaacgatcaaagaggtgaccgcgtccgggtgtattaccgacgacggaagtgtccatcccttggatatactagtgtgtgcaactggattcgatagctcattctgtccgaggtttccaatcgtcggtagaaacggggtaaatttagccgctcagtgggcggaggaaccgaggaattatttatccctagcagtgcacaatttcccgaattacttcatgtttttgggaccaaattgccccattggttcaggccctatcatggtttgtatcgaagcagcgtgcgagtacatggccaattttatgaacagatggcagaaagaagatatcaagtttttcgacccgagcgaagaagctattaatgactttatggaacagaaagatctatttatggatatgacggtatggaaaagcggatgcagaagttggtggcgtaacaacgagactgggaaggtcacggccttatggcctggctcaacgctacattacttggaagctctggcgtatccgaggtatgaggatttcgaagtacagtacgcatcacgtaacaggtttgcataccttggcaacggattcagtcaaagacaactggatccatccgcggaccaaacttattacctaaggcagcgtgacgaggggagcgtactttctaatggcatgtccacaaggaacgtcaaggatctaggtgacctattgacccagtgccgttttgggccaggcggctcagagggcaacagcacggag

SEQ ID NO: 119 - DNA, 1722 bp, synthetic construct
  source 1..1722: mol_type = other DNA
atggcagccacgatacaaaaatgcagggacagacccattcatgccaaccgtacgatgagagtgatttgcgtcggagctggtgttagcggtttatacctggcctataagcttcgtaaacacttcactgactacatcttggacatatatgagaagaacgctgacatcggtggtacatggtttgaaaataggtacccaggttgcgcatgtgacatcccagcgcataactacacatacacgtttgaacccaatccaaactggagtgctagttacgcctcagcagcggaaatttttgaatatctatccggatttgcagagaagtacgacttgagacagtttataacttgccaccatgaggttgtaggagccctttggaaagaggaccaagcggcatggagcgtcaaagtgcgtaaaccgaatggggcgatctttgagcaacaatgcgatttccttatcaacggagcgggtatccttaatgcttggcgttggccggacatcgcggggttagattcttttcagggtactcttgtccacactgcgcagtgggacgctgctatagatgtagagggtaaaaaagtgggcattattggtaacggaagctccggcgtacagatcttacctgagattcaacgtagagcccaacatgtcactgccttctctaggggtcctacttggatcacaccgtctccggccatggaacatcacgtttatacggatgaggagaagcaattatttcgtgacaacccaagtaagctattaaatatgaggaaatcatccgaggctataatggcaagtgcaagcttattctcaacttttctaaagggtagtaacgcgcaggacgtcttccgttcccagatggaaatccacatgaaggggaaaatacacgatgataatcttgctaagaaactggtgcctgacttcccttttgggtgcagaaggccgacccctgggactgattacttggagagtctaacaaaaccaaatgttaccatatgtcatgaggccattgaggagattacaccaagcgggtgcgtcaccgcagatgggcaggagcatcttgttcacatcctagtctgcgctaccgggtttgataatacattctgccctaggtttccaatcattggaagagatggggctaaccttgccgaagagtgggagaaggaaccgaggaactacttgagcgtggctgtgaacggctttcccaactattttatgttcactgggcccaactgcccctttggttccgggggcataattgtagggatcgaggccgaggggacgtatatgactaatttctttaatcgttggcagaaggaggacataaagttctttgacccaaaaaaagaagcggtagatgaattcatggaacaaaaagacttattcatggacacaaccgtctgggctaaaggctgtcgtagctggtggaagcataagaagacgggaaaggtgacagccctttggcctgggagcaccttgcactatttggaggcattggcccatccgagatacgacgacttcgaggtgcagtatgcgtgctccaaccgtttctcttatcttgggaatgggttttcccagagacaacttgatcccaccgctgatctagtttattacttgcgtgaatcagatgagggcagcgtgttcgctgatggtatgtctacacgtaatgcgaaggatcttggtggaagcgcaatttgggggatagctaaggggcgtaagataaacccc

SEQ ID NO: 120 - DNA, 1710 bp, synthetic construct
  source 1..1710: mol_type = other DNA
atggcagccactatacaaaagtgtagagatcgtccgatccacgctaacagaacaatgcgtgtgatttgtgtcggtgctggcgtttctggcttgtaccttgcttataaactgaggaaacaattcacagactacatacttgacatttatgaaaaaaatcccgatatcggtggcacttggttcgagaaccgttatcctgggtgtgcctgtgacattcccgcgcataattatacatacacttttgagcccaacccaaattggtctactaattatgcaagcgcagtggaaatctttgaatatttttccgggttcgctaagaagtatgatttgagacagttcatcacgtgccatcacgaggttgtaggcgcgttttggaaggaagatagagcggaatggtctgtcaaggtgcgtaagcccaatggcgcaatctttgaacaaaggtgcgatttcttaatcaatggtgcgggcatacttaacgcctggaggtggcctgacatagccggcttagattcatttcaagggaccttggtgcacactgcgcaatgggacgaagccataaatatggaagggaaaaaagtcggtgtcattggtaatggaagtagtggcgtacaaatattgccgaggatacagcgtagggcccagcatgtcacagctttctcacgtggccctacatggattacgccatccgcaggtatggagcatcatgtctacacagacgaggagaagcaaaggtttcgtgataacccctctgagctgcttgacatgagaaaatctagtgaagctgtaatggcaagtgcaggtttattttctacatttctaagaggcagccatgcgcaagacgtgtttaggtcccaaatggagaaccatatgcgtggcaagatactagacgacaatctggcgaagaaattagtacccgactttccctttgggtgcaggcgtccgacacagccaggcacggattatctagagtctttgacgaggccaaacgtaactatttgccacgaagccatcgaggagatcacgccttcaggctgtgtcacggcggacggacaggaacatttagtcgatatattggtctgcgcaactgggttcgacaacacctttaggccgagatttccgattataggtagagatggcgccaacctaggagaagagtgggaagaggaaccaagaaactacctgtcagtggccgtaaacgggttcccgaattattttatgtttactgggccaaactgtcctttcggtagtggcggcataatcgtagggatcgaggccgaaggcacgtacatgaccaacttcttcaacagatggcaaaaggaagacatcaagttttttgaccccaagaagcaggcagtggatgagtttatggagcagaaagatctgtttatggataccacagtatgggcgaaaggatgtcgttcttggtggaaacacaacaagaccggcaaagttacagccttatggcctggttctaccctgcattatttagaggccctagcccatccgagatacgatgactttgaggtccagtacgcttgttccaacagattttcttacctgggtaacggattcagccagaggcaacttgacccaacagcagacctagtatactatttgagggaaagggatgaaggttctatatttgcggatggtatgagtaccaggaacgccaaagacctaggagggattctaacggaaaagttagcgatgccc

SEQ ID NO: 121 - DNA, 1728 bp, synthetic construct
  source 1..1728: mol_type = other DNA
atgtcatcccatagcaggccaatccatgccaaccgtcaattaagagttatatgtatcggggcaggcgcgagcggtctgtatctagcttataggcttaaaaagagttttacagacttcactctgcagatatatgagaagaatgcagacgtgggcgggacatggttcgagaataggtacccgggttgtgcctgcgatatccccgcacataactacacctacaccttcgaaccaaaccctgcctggagtgccaattacgccacagcgagcgagattgccgcttacttcgccggattcgctgacaaatacgggttgagagagtacatagcatgtcagcatgaagtcgtccacgccgaatgggatgaggacagggccgagtggatggttcgtgtcagaagaagtgccgatggcacggtattagagcagcgttgtgactttctgataaacgcagcagggatcttgaacgcttggaagtatcctgatataccggggttggatacattcgaggggacccttgttcatacggcccactgggacgaaacgctagacgtaggaggcaaaagagttggcttgattggcaatggttccagtggcgtccaagtgttaccggagctgcagcgtaaggctcagcatgttacggcgttcataaggtcagccacatgggtttccccggcgaggggtatggagtatcatcaatacacagaggaggagaaacgtaacttcagagaaaatcctgaccaactacttgcagtgaggaaacagaccgagggctttatgactagtgcggctcttttctctatgttcatacagggttccaaggctcaggaggtgttagcacaagatatggaaacccagatgagagcgaagctggggtccgatcacgtgctggctaaatccataatacccgatttccctttagggtgcaggcgtcccacacccgggatcaactacctagaatctttgggccagcccaatgtaacagcgtgtcccggacctatcaaagaggtgaccaggactggatgcattgcagcggatggatctgcacacgagttggacgtcttagtctgcgcgacaggcttcgacacgaccttcaggccaaggtttccagttttaggaagagaggcacgtgatttggccacccagtgggcagatgaacccagaaactaccttagcttagcggcaaggcattttccgaattacttcatgtttgtaggcccgaattgcccgataggcagtgggccgattatggtatgtattgaggctgctggcgattatatggcacgtatgttaaacaggtggcagaaggaggggatcaggagttttgaacccaaagctgctgccgtggatgattttatgacctataaggacagattcatggcctccaccgtatggcacgcaagagcgggttccggtcgttgcaggagttggtggagagatagcaggtccgggcaggtgaccgcgttgtggccagggtcaacagttcattaccttgaggcccttgctaccccgaggtatgacgattacgacgtagattacgttggcgggaatcgtttcgactatctagggaacggtttcgcccagaggcaattggctgcaaacgccgacccagtatggtatttgaggacggcagacgagggggaacttttgccagacttgatgtcaacagaaaacgcgaaagacttgggctcccttctaagagatggcttcgagtggacacctgcgcagggcgcc

SEQ ID NO: 122 - DNA, 1563 bp, synthetic construct
  source 1..1563: mol_type = other DNA
atggcagccaatgtctcccaacaaaagttgagatgtgtcatcatcggggctggtgtttcagggattcttatggcttataaactgcaaaagcacctgcgtgaattcatcgactttcaagtattggagaaaaacccggagttaggagggacctggtacgaaaaccgttaccctggctgtgaatgtgatgtgccatcccattgttaccaatactctttcgcccccaatccgagttggagcaaaatctacagccccgcagaggagattcacgcttacttaaagggagtcgcgtctcattttaacttagaaagatatataagatataggacagaggttgtaagtgctaattgggatgaagccacgtccacttggaatgtcgggatttcagatggaagcacaatagcatgtgacgtcctgattaatgccggagggatactgcatgacccgcattggcatgacatcgaaggtttagggtctttcagtggaaagttgatccacaccgctgcatgggcgaaagacttggacttaaaaggtaagagagttgctttaataggctctggttcctctgctgtgcagattctgcctcagcttcaaccgatatgtagctttatcagagtatacatcagaacaccgccatggattgcccccccaataatagagacggatttggaatctccaaacagggagtacacatctgatgaaaaggcaagttttagacagaatcccgaaaagtatagccagttgaggaacgggttaaactctcagattaacggaatgtttcgtgctttgtttaagacgagtccggaacaacattacttacgtcagatgcttgaagccagaatgcgttctctgatcagatccgaagaacttcaagaacacttgattccaaggttcgaggtaggttgccgtagagtaaatccgtgcgagcagtttcttcgttccatacaagaggaaaacgtaacagcagtctttagcgcgattgaagaggtcacgcagaatggtattgtgagtgaaggtgtagaacacccggtggatacgatagttgccgcgacaggttttaataatagctttaggccaaagtttcccttgagaggcaggtatgaggtcaatttacaggatttgtgggccaatgacccaatttcatacttcggcactggcgtagcggggtttcccaattacttgacctttctaggcccaaacacaccaatcgctaacggagcttttatgggccctcttgagggtacctctgattattttattagactacttcaacgtgctattagatttggtgctctgtcatttgacgttaaagcagaagcccaggctgattttgacagccgtatccataaatttatggagaatatggcttggaccggcagttgtagaagctggtataagtccggaagggatgggaagataacagccctgtggcctggtagttcattacactacatgcaagtcttagccgaggacaggtgggaagattacaattgggtgtataagaggcctagatacgactactggtgtaacggcctgtcctggattgaggacccggaaactgcaggt

SEQ ID NO: 123 - DNA, 1010 bp, synthetic construct
  source 1..1010: mol_type = other DNA
atggcagccgacaagattgtgccacagaccgacgagatttttaacgactttaaaattatcgacgaaccgttcgttaccagggacaatcatcccattttagcccacatcttactacccaaggaagcgccccctaaaccgtcccctgtaatcattaattggcacgggggctacctagtcgctgggcacgggcttttcgccctatttttcccgcctttgcttctacagcttgcaaagaaacattctgctattatagtgagtcctgaccacactctgctgccgcataaggacggtctggtcgcggtacaggaggacgccgaagcatttcatgaatggttacattcttctttccctaaagtgttagctgagcgtaccccgggatttcaagtagacctttccagaatacttttacatggcggcagttctggtgcataccttgcccttagttcagctttagcccatccagacagttatagaagtttgtctttaatgtatcctatgattgactttgacacagactggtggagacgtggatctcgtgcagtgggagcgcccaacccgggatgtctgccggacgctgcgttccccgcgaacgataatactgtcaaggatcagcttgaaaaatttatgaaggaaccaattgtcacaatgcctggtgccgagagagacgggtttggggcatgggttgcaagagcaggtctgttcccggagttgtttaaccctgggggaaaattagacgacgacgcagcagtttggctaaacaggcgtgtaaaacagggcgcaaaactgccggagagaatttgggtactacatgccgatgaagatacggtggtacccatcgaaaccagtctgactttccaagagactatgaaacagcaacagaggccggtgaggcttgatattgtcaaagggctggatcattgttgtgacatggctatcacggaggactggaaaggcagccaagacccagtaattcttgaagccgccgattggctagtggaaaagtggttaga

SEQ ID NO: 124 - DNA, 1020 bp, synthetic construct
  source 1..1020: mol_type = other DNA
atggcagccgacaatatagtcctacagacctctgagatttttgatgattttaccataatagatcagcccttttggacaagagaaaatcatcctattttggcgcacatcattttaccacgttcctacttgtctagtagttccaagccaagtccagtcatcataaactggcacggggggtaccttgtcgcaggacatgggctgttacctcttttttttcctccgctgctgctatccctagctaagaaacactccgctattataatatctcctgaccatgcactattgcctagaaaggagggtctgtcatcagtccaagcggacgcggaggcgttccatcattggctgcattcaagttttgctgatactttggcggagaaagcgccaagttacgagcccgacctaacaaggatcctattacatggaggatcttctggcgcctaccttgccctttcttccgcccttagtcacccaaaaagttacagatgcttaagcttaatgtacccgatgattgatttcgacactgactggtggagaaaaggaagtagagccgtgggcgctccaaacccgggctgccttccggatgcggcatttccagtggatgaccaggtcgtcatggatcaactagagaagttccgtgagggacctattgttacaataccaggtgccgagcgtgacgggttcggtgcttgtgtagctagagctggactgttccctgagatttttaacccgggtgggaagcttgacgacgactcagcggtatggttgaatagaagggtagagcaggggtcagagttgcccgagcgtatttgggtcctgcacgctgatggcgacaccgttgttccagttgaaacttctctgtcattcgctcagacgatgaagcagcaggatagacctgttagactggatatagtcaagggattggaccattgctgcgatatggcagtaacagaagattggaaaggatctgacgatcccgtggttttcggagcggctgaatggcttgttgagaagtggcttgag

SEQ ID NO: 125 - DNA, 1020 bp, synthetic construct
  source 1..1020: mol_type = other DNA
atggcagccgataaagtagtcttacaaaccaatgaaatctttgacgatttcactgtattcgaccaaccctttgttactcgtgaggggcatcccgtccttgcgcacgtcctactgcccaggtgccttcccggcagtagttctcaacctagcccaataatcgttaactggcatggaggttatctggtgggaggtcacggtgtattggcaatgttcttccctcctctgttgctacgtttagcaaagaaacatagcgctattatagtcagcccagattatgcgctactaccgaggaaggagggcttgccgtctgtgcaagcagatgcagaagcgtttcacgggtggttacacagttccctggccgaaactttagctgagaaagcgccggggagagaggcggatcttacacgtgttctacttcacggtggatcagcgggtgcatatttagcactgtctagcgcgttggcacacccccgttcatacaggtgtctatctttaatgtaccctatgatagattttgacacggattggtggaggagaggttctagggcagttggtgccccaaaccctgccagactaccagacgcagcgttcccagccgatgaccaggcagtggtcgaccaactggagaaattcagagagggtcccacggtgacaatgcccggtgacgaaagggatgggtttggtgcctggttagcccgtgctggtttattttccgagattcttaatcctggtggggggcttgatgacgactctgcagcttggctgaatcgtcgtgtgaaacagggggcagaactgccaggacgtatctgggtattacacgcggacggggatactgtagttcctattgaaacttccctgggcttcgctcagacaatgaagttgcaagggcgtcctgttaggctagacattgtacgtgggctagatcatatgtgtgacatggccgttaccgaggattggaagggcagtgacgatgctgtcgtacttgatgctgctgagtggctggtagaaaagtggttagag

SEQ ID NO: 126 - DNA, 1029 bp, synthetic construct
  source 1..1029: mol_type = other DNA
atggcagcctcaccgccagaaatgccaaccggctatgaatccaaaacaataacatttaagaatataggcgccctttcccttgaattggacattatgtatcctagaggaaagaacccggaacctgtcccggtgttaatacattatcacgggggcttcttagtggtcggtgacaagtactcatttatgccctattggctactgaacgcatgtgcaagcaggaagtggatcttcgtgactccagattataggttgatccccgaggcaacggctcacgaggcggtagcggacgccttagatgcgtataattgggtgaggcagtcattatcccaggcccttgggatgaagatcggacccgtcatcagtgccgggtcatcagccggggcgtatcttgctttaacatccgccagcatgaccagtattaaacctgacgctctagttttgatatacgggatgttggaccccattagcgaaaggtacacgaagcccggaacgaacatttttggaatgccgccaatagactcacagccactgcttgaccaatggaatttattgaaagaaaaaggcgaaacgacagtccatacgggatacccagtgcccagagacccggcgaacgacttcagaatggggctgattaccgcactacactgtatggctcttttcccggacttcatgacaggggtaccggggttgggtagagatatagcccaaaacggcaccagtgtagtaccggccgaggcgcgtcagttatacacaattgctttcgaacgtgggacgggtctgccaaggaccgcgatcttccacgggaagaatgacctagccgttccgtttagtggatctgaggcggctgccgacagtttgcgtaaattaggcgtcgtagtacacaccgaatttccagacgacgctccacactcttttgactcaatggctggtaatgtagacttagaagatagcgacgataccagcacaaagactcccgcttttgacggccttagagggatagttaaattcttagatagcgtggtagcgttt

SEQ ID NO: 127 - DNA, 1032 bp, synthetic construct
  source 1..1032: mol_type = other DNA
atgtcatccacccagtccatagagctgcccgctggttaccttacgaagacaacgacttataagatcattcacagcgtaaacctagaactggatatcgtatatccccaagaaacctccgtggaccccgtaccggtgttaatccactatcatggcggatttttgatcatcggagataggttttccttcttaccgtactggttattaaatgcgtgcgccgcaagaaagtggatttttgtgacccccaactatagattaatccctgaatccacggcgcacgatgcagtcgcggatgccttagatgcgttcctttgggtaaagacgtcactaagtaggacgctaggggtaaccgtcggtcccgttatcagcgcgggaagcagcgcaggtgcctaccttgccatgataagtgcttcagttagttcagtaaaaccagacgctttggtcttgctttacggaatgctggatccctctagtgaaaggtacaccacgcccggcactaacatcttgggaatgcctcctattgatggacagccgattttagaccaatttgaaacgttaaaacagcaggggagggcaaaccctctggcgggctatcctgtaccttcagatcttgcatccgatttcaggatgggcttgatagctgcggctcattgcatgggagtattgccggatcttatgacaagcattcccggcctatccagggacatagctaggcactccacagtagctattcctacagaggcgaagcacctttttccgattgggtgttccggtgatgtcgttttgccccgtacagctatattgcatggaaaaaacgacaatgtggttccgttcgccgcatctcaagtcgccgcggataccctaaggaagatggggaccgaggtctatacggaattcttggacgacgcgtgtcactcttttgacactatggtcggcaatatcaatatagagaaatctaacgccccagaggccagaactccagctttcgacggtcttagaaggataattaagttcctggacgaaattgctcgtcct

SEQ ID NO: 128 - DNA, 1032 bp, synthetic construct
  source 1..1032: mol_type = other DNA
atgtcatccacgcaatccattgagctacctactgggtatcttacgaaaaccgtgacatataagattatacatagtgtcgatcttgagcttgatatagtctacccacaggaaacatcagctgatcccgtaccggttctgatccactatcatgggggctttctgataattggggatagactgtctttcttaccgtattggctattaaacgcttgcgcagccagaaagtggatattcgtcacgcccaactataggctgatacccgaggcaacggctcatgatgcagtggccgatgcgcttgacgcatttttatgggtcaagactagcttgtcacgtactctgggtgtcaccgttgggccggtaatcagcgcgggcagctccgctggtgcatatcttgccatgatatccgcatctgtctcttcagtcaagcctgatactttggtcctgttgtatggcatgctggatccgtcctccgagtggtacacaactcccgggaccaatatttttgggatgcctcccgtagacggacaaaggatcctagaccaatttgagactctaaaacagcagggcagagccaaaccattggctggttatcccgtcccgagcgacccagcttgcgacctgaggatgagcctgatcgcagcagcgcattgtatgggagtcctaccagaccttatgacttccatacctggtctgagcagagatatcgcttgtcactcaaccgtcgctattccggctgaggcgaagtatctgtttcccattggttgcagtggagatatcgccctacctcgtacagcgattctacacggaaagaatgataatgccgttccttttgcggcctcacaagtagctgccgatacactgagaaagttggggaccaaggtatatactgaattcctggatgatgcttcacacagcttcgatacaatgtttggcaacatcgatatagagaaatccgatgccccagaaactaaaacccctgcattcgatggtctgagacgtatcatcaaatttttagacgaaatcgcaaggccc

SEQ ID NO: 129 - DNA, 1053 bp, synthetic construct
  source 1..1053: mol_type = other DNA
atgtcatccgccccacctgaaatgccctctggctacgaaatgaagaccttaccttacaaaaccacaggctctttgagcctgaatctagatattatctaccccctacaggcttcaacaacacccgtccccgttctaatccactatcacgggggcttccttatagttggagacagacagtcattcctgccccattggcttgtaaatgcgtgcgcagcgcgtggttggattttcgtaacccctgattacaggcttcttcctgaaacaaccgctcatgatgccgtcgcggacgcggtagatgcgtatcaatgggtcaggaccagcctagcgagcacgttgcagcatctgagccccgggcctgtaatagcagcgggggcatcagcaggcgcatatttagctcttacctcagcatcacaagctgaacataagcctgacgcactgctattactatacgggatgttggatcctcttcatgaacactttactcatccagtggataacttcttcggtatgacccccagcgattctcgtccgtttttcgatgcgtgggctgcaggtcagcaaaaagatgatccagcggcaactgttagaacaggctatcccatacccgctgatccttccacggacgctaggatgggtcttattgcagctttgcacggccaggccttactacccgattacatggcgggtgtgcccggacttgctggtagagtgagagatgggtccggtggggaggtagttgatctggtgccggagaaggcgagggggttgtttgcaatcgcgtgtggtagggtcgcgggattaccccctactgctgttgtacacggaaggaatgacgttggggtccctgtgacaatctctctatcagctacggaaaccttgagaaggttaggcgtacaagttcacgcagagttcccggacgacgctccacacgggtttgatgcgatgctgggagatgtcgacatcgaaaggtccagaaaagcggaaggtgacacgcccgccttggaaagccttaggagggttttaggatggctagatgccctagtcgggcgtgtt

SEQ ID NO: 130 - DNA, 1020 bp, synthetic construct
  source 1..1020: mol_type = other DNA
atggcagccgacaacataattttacaaacctcacagatttttaatgatttcacgattatcgaccaaccctttctaataagggaaaaccatcaagtactggcacatattctgctaccgaggaattacagcgggagtcctagcaagccctcacctataattatcaactggcatggtggttatttggtggccggacatggccttcttccattattttttccacctctattgttaaggttagctaaaaaacactcagcgatcatagtgagcccggaccacgcactactacccagagaggagggtctttctagtgttcaggctgacgccgaagcattccaccactggctgcacagctcatttgcagaacttctggcagcaacagcgcctagctacgagccggacttaacaagaatacttttacacggtggctcctcaggggcatatttagccctgagcagcgctttaagtcatccaaaaagctacagatgtgtgagtctaatgtaccctatgatatattttgacaccgattggtggaggaaaggttctcgtgcggttggggcgcccaaccccggaagattaccggacaccgctttccccgtagacgaccaggtagttatgcaccaacttgaagaattcaggcaagggcccgtcgtcaccatgcccggagctgagaggaacgtgttcggtgcgtgtgtagcccgtgcaggattatttcttgaaatattcaaccctggaggcaaattagacgatgatagtgctgtgtggctaaacagaagagtagaacaaggtagtgaactaccagagcacatatgggttcttcacgccgactgcgacacagtggttcccatagagacctcactacgtttcgctcagactatgaaaagacaaggcagacctatcaggcttgacatagtgaaagaacttgatcattgctgcgacatggcagtaacagaagattggaaaggaagcgacgacccagttgtcctagatgctgcagagtggctagtcgagaaatggttagaa

SEQ ID NO: 131 - DNA, 1572 bp, synthetic construct
  source 1..1572: mol_type = other DNA
atggcagcccaattgccctcctccactaaaatccttgttgtagggggtggaccagcaggatccacagcagctacactgcttgcccgtgaaggttttgaggtaacattagtagagaaagcaatcttcccccgttatcacatcggagagagtttattaatatcagttcagcctataatcgatctgctgggagcgcgtgaggcggtagaagcccatggcttccaaaggaagaagggcgtactttgggaatggggtggtgagaggtggctttttgactggaaaaaattacgttatgactataccttccatgttaaaagagaggaattcgacgaaattctgctgagaaatgcccagaaaaacggtgttaaagtgttcgagggaatagacatcagtaggctggaatttgatggggaacgtccggtcgcagctaagtggtcaaaatcttctactggcgagagcgggacgatacagtttgaattcctgttagacgcctctggcagggcagggctgatggccactcaataccttagatcaaggatgttcatgaaagcgtttcagaacgtggccacctggggctactggaaaggggcgacaatcccggaggtagaagttgaggggcccatcacagtcggttcaatcccgtacggctggatctggggtatccctctgagagaccagacgatgtctgtaggcctggtcatacaccaggagctattcaaagaaaaaagggctacacaaagtgtggaagaaatttatcacgagggtctaaaggcatccccattgttccaggacgtagtcctgaaaggagctacgttagagccacaaatccgtaccgagacagattattcctacatatcccgtacgcttgctggtcccgggttctttttagtcggcgactctggagcattcattgacccgctactgtcctctggcgttcacttggctatgcatagcgccttgctagctgccgctagtgtcaaaagcatcatcgcaggtgaagtcgatatggcctcagctaccgagttttatcaaaggtgttaccagggccatttccttagatgggccttaatcgtcgcctcattttacgaggtaaacgcaagaaaagaaacctacttctggacggctcagcagttggcccacgaagagttaggggtgttcaatatgagccaggccgatatgaaggatgtctttgccaccatggtttccggcgttgtagacctgggtgatgctcaaaacgcggggcgtctacaaaaaggggctgaaagggtccatcagtatttagacgacgacggaagagaggaagacgtgacggctttgctgcagaagtcaaagcaaagaattttcgagtacctggaccgtgtaaagaacagagactctagagcggcgatgcaaaggtataaagcaggagggaccgaaacgttctcaatgggactagacgcggatggagctgtgggtggcctatatgtaacgaccgaaccaagattggggttactgagaaaagtggtagaggagagagccgaggctgctacagaagccccggcaccggcagcgccccctccagccgttgcggaagtc

SEQ ID NO: 132 - DNA, 1599 bp, synthetic construct
  source 1..1599: mol_type = other DNA
atggcagccgataatcgtattaagactgttgttatattaggaggaggcactgctggctggatgaccgcagcctacttgggcaaagctctgcaaaacacggtaaaaattgtagttctagaagcaccgaccatacctcgtattggagtaggggaagccaccgtgcctaatctgcaaagggcatttttcgactatctgggaatacccgaagaagaatggatgcgtgaatgtaatgcttcttacaagatggcggttaagttcattaattggcgtacacctggcgaagggtccccagacccccgtacactagatgatgggcatacggacacctttcatcacccattcggcctgctgccctccgctgatcagatacctcttagtcattattgggcggcgaagaggttgcagggtgagacagatgaaaactttgatgaagcatgttttgcggacacggccataatgaacgccaagaaagcgcccagatttctagatatgaggcgtgccactaattatgcgtggcattttgatgcgtctaaagtggctgcatttcttcgtaattttgccgtgacaaagcaggcggtagagcatgtcgaagacgagatgacggaagtattaacggacgaacgtggtttcataacggctctacgtaccaagtctggccgtattcttcagggagatttgtttgtagattgttccggattcaggggattacttataaacaaagcaatggaggagccttttatcgatatgtccgaccatttgctttgtaattcagcagtggcgacggcagtaccacatgatgatgagaaaaacggtgtcgaaccatacacatcttctattgctatggaggccggctggacatggaagattccaatgttaggaagatttgggtcaggccacgtttattcagaccattttgctacgcaggacgaggctactttagccttcagcaagctgtggggcctggatcctgataatacggagtttaaccacgttaggttccgtgttgggagaaataggagagcatgggtgaggaattgcgttagtgtcggattggcatcttgctttgtggagccgctggagtccagtggtatttatttcatatatgccgcgatccatatgttagcaaagcatttcccagacaagactttcgataaggtgttagtggacaggtttaatagggagatcgaggaaatgtttgatgataccagagactttcttcaagcgcattactactttagccctcgtgtcgacaccccattctggcgtgccaataaggagttgaagttagccgattcaatcaaggataaggtggaaacgtatcgtgcaggattgccggttaatttgccggtaaccgatgagggaacgtactatggtaacttcgaggcagagttcagaaatttttggacgaacggatcctactattgcatttttgccggacttggcctgatgccgagaaatcccttaccggccctggcgtataaaccccaaagtatcgcggaggccgaattgttgtttgctgacgtaaaaaggaaaggcgacactttggtagagagccttccatccacgtatgatctgctaagacaactacacggcgcaagt

SEQ ID NO: 133 - DNA, 1575 bp, synthetic construct
  source 1..1575: mol_type = other DNA
atggcagccaacacgcgtaatccggacaaagtcgttattgtgggtgggggtacggccgggtggatgaccgcatcctacctgaaaaaggcttttggtgagagagtaagcgttacgttggttgaaagtggcacgattggaacagtgggcgtcggcgaagcaacattctctgacattagacatttctttgaattcctggatttaagagaagaagagtggatgcccgcctgtaacgccacatataaattggccgtcaggttccaggattggcagcgtccaggccaccatttttaccacccgttcgaacaaatgagaagcgttgatgggttccctctaactgactggtggcttcagaacggaccaaccgacagattcgatagggactgttttgtcatggcgagcctttgtgatgcaggcaggtcacctcgttacttaaatgggtcacttttgcagcaggagtttgacgaaagagcggaagagcctgcaggactaacaatgtccgaacatcagggcaaaactcagtttccctacgcataccatttcgaagctgcgcttctggcggaatttctaagcggttatagcaaggaccgtggtgttaagcacgtagtcgacgaggttctagaagtcaaattagacgatcgtggttggatttcccacgtagtgactaaagagcatggagatattggcggtgacctttttgtcgactgtacgggctttaggggagtattgctgaatcaggctttaggggtaccctttgtctcctaccaggataccctaccaaacgatagtgctgtcgcccttcaggtgccgctagacatggaagcaaggggtattcctccatatacgagagcgaccgcaaaagaggccggatggatttggactatcccgctgatcggcaggataggaaccgggtatgtgtatgcaaaagattactgttcacctgaagaggctgagaggactttacgtgaatttgttggtccggaggctgcagacgttgaggcgaaccacataaggatgcgtatcgggcgttcagagcagtcttggaaaaataactgtgttgccattggcttatctagtgggttcgtcgaaccccttgaatcaacaggcatttttttcatccaccatgccattgagcagctagttaaacactttcctgcaggggattggcatccccaacttcgtgcgggctacaacagtgcagtcgccaatgtgatggatggagtcagggagtttctagtcttgcactaccttggggcagcaaggaatgacacaaggtattggaaagacaccaagactagagctgttcccgacgcgcttgctgagaggatagagcgttggaaggtacagttgccggacagtgaaaatgttttcccgtattatcatggacttccgccgtattcctatatggctatcctgttaggtaccggggccattggacttagaccttctccggccctggcactggctgatccagctgcagctgagaaggagttcaccgccataagggaccgtgctagattcctagtagatacattacctagtcaatatgaatactttgctgcgatgggtcagagagta

SEQ ID NO: 134 - DNA, 1557 bp, synthetic construct
  source 1..1557: mol_type = other DNA
atggcagccattaactcagtccttatagttggtggaggaaccgctggatggatgaccgccgcatacctgagtaaggctttcgataagaatattaatattacggtggtggaatcaaaggaagtgaagaaaataggcgtaggggaggcgacgttttcaacggtgagacactacttcgattacctagggctgaatgagagcgaatggcttcctgaatgttctggcagttataagctaggtataagatttgagaactgggacggtcaaggcaatcacttctatcatcccttcgagcgttgggaggtcgtcaaaggcttcccaatttcagaatggtggctatccaagaaattaaaagatcaaagattcgactacgacacattcttgactccgcacttatgcgaggccaaacgttctccgagacgtcttgacggtagtctttttgctcaaagtattgacaagtccttagggcaaagtacattagctgaacaaagggcacagtacccctacgcgtaccatttcgacgcagacggtgtagctagctttttaaagagatacgcaatgaataggggggtaaaacacatagaggacgatgtcacgcatgtcgagattgacactaatgggaatattggctacctggaggccaaaatctatgggaagctgagagctgacttgtatatagactgcacgggctttaagggcctgcttattaataaagctctaaatgaacctttcattagtttctctgatgttttaaagaacaacagagctgtggcattacgtgtcccgagagaaaacgaaaatgacatcgagccgtataccactgcccgtacgatgagcaacgggtggaggtggacaataccactttacaagaggaacgggtatggatatgtctactgtaacaagtaccaaagtcctgaagaggcagaaatggaactgaggaagagcatcccttatgaaaatgctgaatgcgtcgcaaatcatatacgtatgaggatcggcagatctaataggagctgggttaagaactgtgtagcaataggattgtcatccgcgttcgtggaaccgttggagagcactggaatcttttttattcagcatggaatagagcagttagtacgtttttttcctagatcaagcggtaacgaattgctaatagaggaatataatacgagagtcaacagagttgttgatggagtaaaagaatttctattattgcatttcagtcttgcccagaggaacgataccctgtattggaaggaatggaagaatgtagagctgccgaaggagttactgaagaagatcacgcttgcccaggatcacctattagacaaggaaacgatataccctttttaccacggctttgaagaatacagctggaataccatgatcctaggcttgtgctctggatctttaaacaacaagcccgccttgagtcttatgacctctgatgaggccgatgaaatgctgctacagcactacatgaaagcagagaatatggtgaataatttacctacgtgttacgagtacttaaaacatattcacgacctaaagaataaaaat

SEQ ID NO: 135 - DNA, 1596 bp, synthetic construct
  source 1..1596: mol_type = other DNA
atggcagccgcctgtacgggatggactagtcccttacctctatgtgtttgtttactattgacatgcgggtttgccgaggcgggtaagttgctggttgttcctatggacggaagccactggtttacaatgagatcagtcgttgagaaattgatcttgaggggtcacgaggttgtggtggttatgcctgaagttagctggcaactgggtcgtagccttaattgtacggttaagacctactcaacgtcttacacgttggaggacctggacagggagtttaaggcctttgcccatgctcaatggaaagctcaggtcagatcaatctattctcttctgatggggagttataacgatatatttgatctttttttttccaattgtcgtagtttgtttaaagacaaaaagttggtagaataccttaaggaatccagtttcgacgctgtctttttggatcctttcgataattgtggcctgattgtcgcgaagtactttagccttccatcagttgtattcgcacgtgggattctgtgtcactatcttgaggagggagcccagtgccctgccccgctttcctacgtccctcgtattttattaggctttagtgacgcaatgacattcaaagagcgtgtaagaaatcatattatgcacttagaagaacatttactatgccatagattttttaaaaacgctcttgagatcgcatctgaaatacttcaaacgcctgtcacggaatatgatttatattctcacacgagcatctggctgttgagaactgactttgtgctggattacccgaaaccagttatgccaaatatgattttcatagggggcataaactgccaccagggaaagccacttccaatggaatttgaagcatatatcaatgcctccggtgaacatggaattgtggtgttctccctgggttcaatggtcagcgaaatccccgagaagaaagcaatggctatcgccgatgctctgggaaaaataccacaaacagtgctttggaggtataccggaacgcgtccgtctaacctagctaataatacaattctggtcaagtggctgcctcagaacgaccttttggggcacccaatgactcgtgcatttattacccatgcgggctctcatggggtttacgagagtatctgcaatggtgttcccatggtaatgatgccacttttcggcgatcaaatggataacgcaaagcgtatggagacgaaaggtgctggcgtaaccttaaatgttttagaaatgacttcagaagatcttgagaatgccctaaaagctgttattaatgacaaatcctacaaggagaacattatgaggttatcttcattgcataaggacagaccagttgagccgttggatttggcagtgttttgggtcgaatttgtaatgcgtcataagggagcaccccacttgaggccagcggcccatgatctaacgtggtaccagtatcatagcttggatgtaataggcttcctattagcagttgtccttacagtggcgtttatcacctttaagtgttgtgcttacggttacaggaagtgcctaggaaagaaagggagagttaagaaggcgcacaagtctaagacgcat

SEQ ID NO: 136 - DNA, 1590 bp, synthetic construct
  source 1..1590: mol_type = other DNA
atggcagccgctcccgtcgccttcccaacttcctttttcttatgtttgttgcttgcatctgggctagctcaagcagggaggttattagttgtcccaatggacggcagccactggtttactatgcaaatggtcgtagaaaagctgattcatagaggacacgaagtagtggtcgtcatacccgaggtttcctggcagttaggcaaaagcctaaattgcaccgttaaaacctatagtatatcacacaccttggaagacctggacagagagttcaaatatctttcatatacacagtggaagactcccgagcatagtatcaggagctttctgactggcagtgcgagaggcttttttgaattaacgttctcacattgtagaagtttgtttaacgataagaaattggtcgagtatctaaaacaaaggttttttgatgcggttttcctagatccctttgatgtctgcggcttaattgtcgcgaagtatttttcccttccgtctgtcatattcgccaggggcgtattttgcgactacttggaagaaggagctcagtgccctagcctaccttcctatgtcccgaggcttttctctaaatatacagacaccatgactttcaaagagagggtctggaaccacttgatttacattgaagagcacgcattctgcagctattttttgcgtactgctgttgaggtcgcaagtgagatattacaaacccccgtaacaatgaccgacctatttagtccggtcagcatttggttgcttagaacggacttcgtactagagtttcctagacccgttatgcccaatatggtttttataggcggaatcaattgcctgcagaaaaaatccttaagtaaggaattcgaggcctatgtcaacgcttctggtgagcacggtatagtggtttttagtcttgggtctatggtctcagagatacccgagaaaaaagcgatggaaatcgcggaagcgcttggaaggattcctcagacagttctttggaggtacacggggacgaggccgagtaaccttgctaaaaatacgattcttgtaaaatggttgcctcagaatgatcttttgggtcacccgaagacgagagccttcattacgcacagtgggagtcacggtatctacgagggcatatgcaatggcgttccaatggtaatgatgcccctttttggggatcaaatggataacgcgaaaagaatggaaactcgtggtgccggcgttacccttaatgttctggaaatgactgccgacgacttggagaatgcactgaagactgtcataaataacaaaagttataaagaaaacataatgagactgtctagcttacacaaagaccgtcctatagaacctctggatttagccgtattttgggttgagtatgtgatgagacacaagggtgccccgcaccttaggcccgccgcgcacgacttaacctggtatcaatatcactctttggatgtgataggcttcttactggctatagttttgactgtcgtcttcattgtcttcaagtgctgtgcgtatgggtgcaggaagtgtttcggagggaaggggagggtcaaaaaatcacataaaagtaagactcac

SEQ ID NO: 137 - DNA, 1596 bp, synthetic construct
  source 1..1596: mol_type = other DNA
atggcagccgcccgtgctggttggacctccccagttccgctatgtgtatgcttgcttcttacatgtggatttgctgaagcgggaaaactgcttgtagtccctatggatggatcacactggtttacgatgcaaagcgtagtcgagaagttaatcctgagggggcatgaggtagtcgtcgtgatgccggaggtgagctggcagctagagagatccttaaattgtaccgtcaagacgtattccacctcttacactttggaggaccaaaatcgtgagttcatggtgtttgctcatgcgcagtggaaggcgcaggcgcagtcaatattttcactgctaatgtcaagctcttctggattccttgatctattcttttcacattgcaggtccctgtttaatgacagaaagcttgtggaatacttgaaggaatcctcttttgatgctgtatttctggatccgttcgatacatgtggtctaatcgtagcaaagtatttcagccttccctccgtggtttttactaggggaatcttctgccaccatctggaagagggagcccagtgccccgccccgctatcctatgtcccaaacgacttgctgggttttagtgacgcgatgacctttaaagaaagggtatggaaccatatcgtacatcttgaagaccatctgttttgccaatacctgttcaggaacgcgctggaaatagcgtcagaaattttacaaacccctgtaacagcatatgacttatattcccatacaagtatctggctactaagaacagacttcgtccttgactatcccaaaccagttatgcctaatatgatatttatcggagggattaattgccatcaaggtaaacccttgccaatggagttcgaagcctacataaatgccagcggggagcatgggattgtggtttttagcttaggaagtatggtaagtgaaatcccagaaaagaaagcgatggcgatagccgacgctcttggcaagatacctcaaactgtgctgtggagatatacgggtacgcgtccctctaacttggccaataacactatcttggtaaaatggttacctcagaatgatctattgggacatcctatgacccgtgcatttattactcacgcaggtagccatggagtttacgagtcaatctgcaacggtgtgccgatggtaatgatgccacttttcggcgatcagatggataatgccaagaggatggagacaaaaggtgctggggttaccttaaacgtcttagagatgacaagtgaggatcttgagaatgcgctgaaggcagtcatcaacgataaaagctataaagagaacatcatgcgtcttagctctctgcacaaagatagacccgttgaacctctagatttagccgtattctgggtagagttcgttatgaggcacaagggtgcgccacatttaagaccggccgcgcacgatctgacgtggtatcagtatcatagtctggacgtgatagggtttctacttgcagttgtactgacagttgcctttattaccttcaagtgttgcgcgtacgggtatcgtaagtgtttagggaagaaggggcgtgtgaaaaaagctcataagtccaaaacccat

SEQ ID NO: 138 - DNA, 1599 bp, synthetic construct
  source 1..1599: mol_type = other DNA
atggcagccgcccctgcggattttccggcaagtctgcctctgtgcgtttgtcttctactagcttctggcctagcgcaagcaggaaggctattggttgtgcccatggacggatcccattggtttaccatgcagactgtagtggagaagctgctgcataagggtcacgaggttgtagtggttgtacccgaggtctcatggcagcttactaagccccttaatttcgttgtcaaaacctatgctgtctcccacacccaagaggacctgaatagagagttcaagatttttatcgacgcacagtggaagtctcagcaggaaggagggatcctgccacttttagatagtcccgctaagggattttttgagttgctattctctcactgtcgttcattgttcaacgataagaaattagtagagtatttgaaacagacgtcttttgatgccgtgttcttagatccttttgatgtgtgcggcctgacggtcgctaaatacttctcattgccaagtgtcgtcttttctcgtgggatattttgtcactaccttgaagatgcagctcaatgtcctagcccgcccagttacattccacgtatgctgttgaagtttactgacaccatgacgttcaaggaacgtaccaggaacttattggcttatatgggcgagagagcgttttgccacaagttcttcaagtctgcagcagacatcgcttccgaagtattgcaaactcccgtgactatgacggacctatttagcccggtctcaatatggctactgcgtaccgactttgtgctagaatttccacgtccggttatgcccaatgtaatatatatagggggtatcaactgccatcagggtaaaccactgtccaaagagttcgaagcgtacgtgaatgcttccggcgaacatgggattgtcgttttcagcctgggttctatggtgtctgagatccctgaaaagaaagcaatggaaatagccgaagctttagggcgtattccacaaacagtactttggcgttatacgggaactagaccttcaaaccttgcaaaaaacaccattcttgttaaatggttgcctcagaatgacttactgggtcacccgaaaaccagggctttcataacccacagtggttctcacggtatatatgagggtatctgtaatggcgtcccaatggtaatgatgccattgtttggagatcagatggacaatgcgaagagaatggagactaggggtgccggtgttacgttgaacgttttggagatgacagcggacgacctagagaatgcactaaagacggttattaacaacaaatcttataaggaaaatataatgcgtttatcctccttgcacaaagatagaccaatcgaaccgctagatttagcagtattttgggtggaatacgtgatgagacacaagggcgcaccccatcttcgtcccgcagctcatgatctaacatggtaccaataccatagccttgatgtgataggatttttgttggccatcgtactgactgtcgtctttatcgtctttaagtgttgcgcctatgggtgccgtaaatgcttcggaggtaaaggacgtgtaaagaagtcacataagagcaagacccat

SEQ ID NO: 139 - DNA, 2064 bp, synthetic construct
  source 1..2064: mol_type = other DNA
atgactacgctgctaaatccttatttcggagagtttggaggaatgtacgtgcctcaaatcctaatgccggccctaaggcaacttgaggaagcattcgtcagcgcacagaaagaccccgagttccaggcacagttcaatgacctattgaagaattacgccggtagacctactgcattaactaagtgccagaatattaccgcgggaactaacaccacgctatatttgaagagagaggacttactacatggcggggcacacaaaacaaaccaagtacttggccaggctctgctggctaagagaatgggcaaaacagaaattatcgccgagactggggcaggccaacacggcgttgccagcgctctagcatcagcgttactgggactgaaatgtagaatttatatgggcgcgaaggatgtggaacgtcagtcccccaatgtctttaggatgaggctgatgggtgccgaggttattcctgttcatagtggttccgccactcttaaagacgcatgcaacgaggcgcttagagactggagtgggagctacgagacggcccattacatgctagggaccgccgcaggtccccacccgtatccgacaatagttagagaatttcaaagaatgataggagaggaaaccaaggctcagattttagagagggaaggccgtttgccggatgccgtaattgcttgtgtaggtggtggaagcaatgcaataggaatgttcgccgattttattaatgagacgaatgtcggcctaattggtgtggaaccaggcggacacggaatcgaaacgggtgaacatggtgcaccacttaagcatggaagggtgggcatctatttcgggatgaaagccccgatgatgcaaaccgaagatgggcaaatagaggagtcatattctattagcgccggactggacttcccaagcgtaggtccgcaacatgcatacctaaacagtacggggagggctgactacgtgagcataacggacgacgaagcactggaagcttttaaaacactgtgtttgcacgaaggaataattccggcgcttgaatctagtcatgctcttgcccacgccctgaaaatgatgagagagaaccctgataaggaacaattgttggtggtgaacctaagtggtaggggcgataaggatatattcaccgttcacgacatactaaaggcacgtggcgagatcgggggcggttctggtgagggacgtgggagcctattgacttgcggcgatgtcgaggagaatcccgggccaatggaacgttacgagtcactttttgctcaactgaaggagagaaaggagggggccttcgtgccgttcgtgacccttggggacccagggatcgagcaaagccttaagatcattgatacccttattgaggctggggcagacgctctggagttggggataccattttctgaccctttagcggatggccccactattcagaacgctacgttacgtgccttcgcggcgggcgttacaccggcacagtgcttcgaaatgttggctctgataaggcaaaaacacccaaccatccccatagggttactaatgtatgccaacttagtgttcaataaggggattgatgagttctatgcgcaatgtgaaaaggtgggcgtcgattcagtgctggtcgcggacgtacccgtagaggaatccgctcccttcagacaggccgcgcttcgtcataacgttgcaccgatatttatatgccccccaaacgcagacgacgatttgttacgtcaaatcgcctcttacgggcgtggatacacttacttgctaagtagggctggggtaactggggctgagaaccgtgctgctctacccttgaaccatttagtcgctaaactgaaagagtataatgctgctccacccctgcagggctttgggataagtgcaccagatcaggtaaaggcggctatcgatgcaggagcggcgggcgcgatatcaggcagcgcgatagtaaagattatagaacaacacataaatgagcccgaaaagatgctggccgcgttgaaggtattcgttcagcccatgaaggctgccactcgttca

SEQ ID NO: 140 - DNA, 1275 bp, synthetic construct
  source 1..1275: mol_type = other DNA
atgggaggtgcgagtgccagcggtgtagacaataaatttaataaggaaagggtgatcgctattggtgagataatgagattaccaaatcttaattctttacaggtcgtcgcattcatcaattcactaagggatgatccgagccaaagcgccaacctacttgccgaagcgaagaaattaaacgacgcccaggctcccaaaggaggtagcagcgcaagttctgcaggaggcagtagtgttgacaacaaatttaacaaggaagcgcaaactgcgggtgtggaaatcatggaactgccaaacctgaatacacggcagctcctggctttcatccagtcattacgtgatgatccgtcccagtcagcgaatttgctggcagaggctaaaaagctcaacgatgcgcaagctcctaaaaccagtggaggcagttccgcctccagcgcaggagggtcaagtgtagacaacaaattcaacaaagaaagagtgattgcgataggtgagatcatgcggttacctaacttaaacagtctccaagtggtggccttcatcaatagtttacgggatgacccaagccaaagcgctaacttactagcagaagctaaaaagctaaatgatgctcaggcaccgaaaggaggttctagtgcgtccagtgcaggagggagcagtgttgataacaagttcaacaaggaggctcagaccgcaggggtggagattatggagctacctaacctaaacactagacaacttttggcgtttatccagtcccttagggacgacccatcccaatccgcaaatctattggcggaggctaagaagctgaacgatgcacaagcccccaagacaagtggtagcggtgcatctggtagtggtgcaggtggaagcagcgtagacaataagtttaataaggagcgtgtaatagccatcggagaaattatgcgtttgccaaatctgaactccctacaggtggtggctttcataaatagtttacgtgatgacccctcacaatccgcgaacttgttagctgaggcaaaaaagttaaacgacgctcaggctccaaaaggttccggtgcctccggaagcggagcaggtggatctagcgttgacaataaatttaacaaagaggcgcagactgcaggcgtggaaattatggaattgccgaacttgaacacccgtcagcttttggccttcatacagagcttacgtgatgacccatctcaatcagctaacctattagcagaggctaagaagttaaacgatgctcaggccccaaagacaagttaa

SEQ ID NO: 141 - DNA, 210 bp, synthetic construct
  source 1..210: mol_type = other DNA
atggtagacaacaaattcaacaaagaactgggttgggcgacctgggagatcttcaacttacctaacttaaacggtgtgcaagtgaaggccttcatcgatagtttacgggatgacccaagccaaagcgctaacttgctggcagaagctaaaaagctaaatgatgctcaggcgccgaaaggttcaagcagctcaggttcgagttcttccggc

SEQ ID NO: 142 - DNA, 210 bp, synthetic construct
  source 1..210: mol_type = other DNA
atggtagacaacaaattcaacaaagaaacaatacaagcgagtcaagagatcagactattacctaacttaaacggtagacaaaagctcgccttcatccacagtttacttgatgacccaagccaaagcgctaacttgctggcagaagctaaaaagctaaatgatgctcaggcgccgaaaggttcaagcagctcaggttcgagttcttccggc

SEQ ID NO: 143 - DNA, 168 bp, synthetic construct
  source 1..168: mol_type = other DNA
atgcaactgctgagatgttttagcatcttttccgttattgcgagcgtgttagctcaggaacttactactatctgcgaacaaatcccaagccccacgctggaatcaaccccttactcactgagtacaacaacgatacttgcgaacggcaaacttgacaagagaggaagt

SEQ ID NO: 144 - DNA, 216 bp, synthetic construct
  source 1..216: mol_type = other DNA
atgaaggtgcttgatttgctaaccgtgctgagtgcttcctcactactgagcaccttcgcagcggcggagtcaacggctactgctgatagcacaactgcagccagtagtacggcatcttgtaatcctttaaaaactactggatgcacacctgacaccgcgttagccactagcttcagcgaagacttctcatcttccagtcttgacaagcgtgggtcc

SEQ ID NO: 145 - DNA, 153 bp, synthetic construct
  source 1..153: mol_type = other DNA
atgttatcactgaagactttgttgtgtactcttctaacagtctcaagcgtgttggcgacgccagttcctgccagggacccctcctccattcaattcgtccatgaagaaaacaagaagaggtactatgactacgaccacgggtcccttggcgaa

SEQ ID NO: 146 - DNA, 99 bp, synthetic construct
  source 1..99: mol_type = other DNA
atgaagtttgcgtatagtttactgcttccccttgccggtgtttcagcctccgtcatcaactataaacgtgacggcgactcattagacaaacgtggaagt

SEQ ID NO: 147 - DNA, 198 bp, synthetic construct
  source 1..198: mol_type = other DNA
atgaagctacagtctcttctagtgtctgcagcggtacttacatccctaacagaaaatgtaaatgcatggagtcctaataattcctatgtcccggctaacgtgacctgcgacgacgacataaatctagtccgtgaggcatctggccttagcgacaacgaaactgagtggctgaagaaacgtgacgcttatacgaaagag

SEQ ID NO: 148 - DNA, 60 bp, synthetic construct
  source 1..60: mol_type = other DNA
atgctattgcaggctttcttgttcttgctggcaggattcgcggcaaaaatcagcgcatct

SEQ ID NO: 149 - DNA, 297 bp, synthetic construct
  source 1..297: mol_type = other DNA
atgaaactgtccgctacgactctaactgcggcaagcctaataggctattctacgattgtgtctgctcttccgtatgcggcggacattgacacaggatgtacgacgactgcgcacggttcccatcagcacaagagagctgtggctgtaacctacgtgtacgagacagttaccgtggataaaaatggtcaaacagtaacgccgaccagtaccgaagccagtagtaccgtcgccagcaccacaacgcttattagtgaatcctccgtcacaaagtccagctccctggataaaaggggttct

SEQ ID NO: 150 - DNA, 276 bp, synthetic construct
  source 1..276: mol_type = other DNA
atgcgttttccttccatctttacggacgtgttgttcgctgcgtcaagcgccttagccacccctgttaacacgaccacagaagacgagacagctcagatccctgccgaggcggttataggttacagtgaccttgaaggggacttcgatgttgccgttttgccgttttctaattccaccaataacgggttgctattcataaataccacaatcgcgtcaatcgcagctaaggaagaaggagtaagtttagagaagagggaagcaacgaatttcggatca

SEQ ID NO: 151 - DNA, 300 bp, synthetic construct
  source 1..300: mol_type = other DNA
atggaagagcaaagagagatattagaacagttgaagaaaactttacagatgctgactgttgagccgtccaagaataatcaaatcgccaacgaagaaaaagagaagaaagaaaacgaaaatagctggtgcatcttggaacacaattacgaggatattgcacaagagtttatcgactttatttataagaaccccacaacctatcacgttgtttctttcttcgcggaattgctggacaagcacaacttcaaatacttatcagagaaaagcaactggcaggattccatcggagaagatggcgga

SEQ ID NO: 152 - DNA, 225 bp, synthetic construct
  source 1..225: mol_type = other DNA
atgttcagaatacaattacgtaccatgtcatctaagacgtgcaagagtgactaccctaaggagtttgtctcattcctgaatagcagtcacagtccatatcataccgttcacaacattaagaaacatttagtaagtaatggattcaaggagctaagcgagcgtgactcatgggctggacatgtggcgcagaaaggcaagtatttcgttacacgtaacgggagtagt

SEQ ID NO: 153 - DNA, 891 bp, synthetic construct
  source 1..891: mol_type = other DNA
atgtctgacagcagtccgactattaactttattaactttaaccaaaccggtacatgtatcagtttaggaacgtccaaaggcttcaagatattcaactgtgaacctttcggcaagttctattcagaagacagtggcggatatgccattgtagagatgctattcagtaccagtcttctagccttagttggcataggtgatcaacccgctcttagccctaggcgtctaaggatcatcaatactaagaaacactctatcatctgtgaggtgacgttcccgacttccattttgtccgttaagatgaataagtcacgtcttgtcgtacttttgcaagagcagatatatatctatgacataaacacaatgaggcttctacacactattgagaccaatccgaacccaagaggtttgatggcgatgtcaccaagtgtagccaacagctatctggtgtacccttctcctcctaaagtaattaacagcgagattaaggctcacgcaacaacgaataatataacactgtctgtcggtggaaacactgagacgagttttaaacgtgaccagcaagacgcgggccatagcgacatctctgatctggaccaatactctagcttcactaagagggacgatgctgaccccacgtcttctaacggtggcaatagcagtattattaagaatggcgacgttatagttttcaacctggagactcttcagcccacgatggtcatcgaggcacataagggtgagatcgcggcaatggcaatttcctttgacggaacacttatggcaacagcgtccgacaaggggactataattagagtatttgatatagaaacgggagacaaaatctatcagttccgtaggggcacatacgcgacaagaatttatagcataagc

SEQ ID NO: 154 - DNA, 171 bp, synthetic construct
  source 1..171: mol_type = other DNA
atgattgcccttcctgtagaaaaagcaccaaggaagtctctttggcagagacacagggccttcatctccgggatcgtcgctctaattattataggtacgtttttcttaacttctggcttgcatcctgctccaccgcatgaagcaaaacgtccccatcatgggaaaggaccc

SEQ ID NO: 155 - DNA, 105 bp, synthetic construct
  source 1..105: mol_type = other DNA
atgtttagcctgaaggcattattgccgctggcgttgttacttgtctctgccaatcaggttgccgccaaggtccacaaagcgaaaatatacaaacacgagttgtcc

SEQ ID NO: 156 - DNA, 297 bp, synthetic construct
  source 1..297: mol_type = other DNA
atgaaattggagaacactttatttacacttggtgcactgggttccatttctgctgcgttagtaattccgaatctggagaatgcagccgatcaccatgaattgataaataaagaggaccaccatgagaggcccaggaaggttgaatttacaaaggatgacgacgaggaaccttctgacagcgaggacaaggaacacgggaaattccacaagaagggtaggaaaggtcaggacaaagagtctcccgaatttaatggtaagagggcctcaggtagccacgggtccgcgcatgaaggagga

SEQ ID NO: 157 - DNA, 492 bp, synthetic construct
  source 1..492: mol_type = other DNA
atgaaagcgttcacatctcttctgtgcggtttgggtttgtcaacgaccttggccaaagccatttccctacaaaggccgttaggcctggacaaagacgtgcttcttcaagcagcggaaaagttcgggttagacttggatctggatcatttgctaaaggaactggacagcaacgtcttggatgcgtgggctcaaatcgaacatctatacccgaatcaagtaatgtctctggaaacgtcaaccaaaccgaaattcccagaggcaatcaagaccaaaaaggactgggacttcgtagttaagaacgacgcaatcgagaactatcaactgcgtgtcaacaaaattaaagatccaaagattttgggaatcgaccccaacgtaactcaatacacgggctacttggatgtcgaagacgaggataagcacttcttcttctggacttttgagtcaagaaatgaccctgcgaaagatccagtgatcttgtggctgaatggtgga

SEQ ID NO: 158 - DNA, 225 bp, synthetic construct
  source 1..225: mol_type = other DNA
atgaccaaaaactttattgttacgttgaagaaaaatacgccggacgtggaagcaaagaaattcttggatagcgtccaccatgctggagggtcaatcgtccataagttcgatataatcaaaggctacacaataaaagtgcccgatgtcctgcacctgaataaactaaaggaaaaacacaatgacgtgatagaaaatgtcgaagaagacaaagaggtacatacgaac

SEQ ID NO: 159 - DNA, 234 bp, synthetic construct
  source 1..234: mol_type = other DNA
atgaaatcttttattactagaaacaaaactgccattttggcgacggttgccgccactggtactgctattggtgcctattactactacaaccaattacagcagcagcaacagcgtggtaagaaaaatacaattaacaaggatgagaagaaggacacaaaggattcacagaaggaaacagaaggcgcaaagaagtctactgcaccttctaacccaccgatatacccagttagctct

SEQ ID NO: 160 - DNA, 351 bp, synthetic construct
  source 1..351: mol_type = other DNA
atggcgatgttctgtaccttctttgagaaacatcatagaaaatgggacatcttactagaaaagagcaccggagtgatggaggcgatgaaagtaacttcagaagagaaagagcagttgtctacagctatcgatagaatgaatgaaggtctggacgcatttattcaactatataacgaatccgagatcgatgaacctttaatccagttggatgacgatacagcagaactaatgaaacaggctagggacatgtacggccaagagaaacttaacgagaaattaaacacaataatcaaacaaatcctgtcaatttctgtctccgaagagggtgagaaagaaggaagcggatcaggc

SEQ ID NO: 161 - DNA, 513 bp, synthetic construct
  source 1..513: mol_type = other DNA
atggctgacagggacaggagtggcatttacggaggtacctatggccaacaccagcagggaggaggtagacccatgggcgaacaggttaaaggaatgattcaggacaagggtcccactgcctctcaggcgcttactgtcgcaaccttattccctctgggcggtctacttttggtcctatctggattggccttagccgcaagcgttgtcggtttagctctggcgacccccgtatttctactattttctcccgttcttgtcccggctgctttactaatcggtaccgctgttacggggtttctgacctcaggtgctctaggactgggaggactttctagccttacttgcttggccaataccgctagacaggcgtttcagaggactcctgactatgtagaggaagcccgtcgtagaatggcggaagccgctgcacatgctggccaaaagacggctcaggcaggacacgccatccagtcaagggctcaagaggctgcatctggaggcgcaggtgctgga

SEQ ID NO: 162 - DNA, 279 bp, synthetic construct
  source 1..279: mol_type = other DNA
atgtctacgctacagagaaggcgtgttaacagagcagactctggtgacacgtccagcatccacagctccgcaaataatacaaaaggtgataagattgcaaacatcgctgtcgatggcgatgacgataacggtacaaacaagaaaatagcatacgatcccgaagagagtaagctaagagataacataaacattcctactttgactttaatggaggaggttcttttgatgggtttacgtgacagagagggatacctatccttctggaatgattccatcagt

SEQ ID NO: 163 - DNA, 264 bp, synthetic construct
  source 1..264: mol_type = other DNA
atgtcacttcccttgagaacgatagtacatttggtaaagccctttgcttgcactgctaggtttagtgcgagatacccaatccacgtcattgttgttgctgttttattgagtgccgctgcttatctatccgtgacacaatcttaccttaacgaatggaagctggactctaatcagtattctacatacttaaacataaagccggatgagttgtttgagaaatgcacacactactataggtctggtggaaacgctgcaattagaagc

SEQ ID NO: 164 - DNA, 87 bp, synthetic construct
  source 1..87: mol_type = other DNA
atggatagcaaagggtcctcccagaagggatcaagattgcttctgctgctagtatccaatcttttattgtgccagggggttgtatca

SEQ ID NO: 165 - DNA, 69 bp, synthetic construct
  source 1..69: mol_type = other DNA
atgaaagcgttcacgtccttattatgtggcttgggtttgagcacgacactggctaaggccatctcactg

SEQ ID NO: 166 - DNA, 753 bp, synthetic construct
  source 1..753: mol_type = other DNA
atggtatctaaaggagaagaattgtttacaggcgtggtaccaattctggttgaattggacggtgacgtgaacggacacaaattcagcgtgagtggagaaggcgagggagatgctacctatggcaagttgacgcttaaactgatctgcacaacgggcaaattaccagtgccctggccgacgcttgtaacaactcttggatacgggttacagtgctttgcccgttatccagaccatatgaaacagcatgacttcttcaaatctgcgatgccggagggatatgtacaggaacgtacgattttctttaaggacgatgggaactacaagactcgtgctgaggttaagtttgaaggcgacactctagtcaataggatagaattaaagggtattgattttaaggaggatgggaacatcctgggccataaactagagtacaactacaattcacataatgtctacatcaccgctgataaacagaagaacgggatcaaagctaatttcaagatacgtcataatatcgaagatggtggcgtccagcttgctgaccactaccagcagaacacgcctataggcgacgggccggtgttgctacctgacaatcattatctgtcctatcagtccgccctttcaaaagaccctaatgagaagagggatcatatggtgcttttagaatttgtaaccgcggcagggatcacacttgggatggatgagctgtataaaggaggtagtggaagtgcgggatctggcggtagtagc

SEQ ID NO: 167 - DNA, 1161 bp, synthetic construct
  source 1..1161: mol_type = other DNA
atgaagatcgaagaaggaaagttagtgatctggataaatggtgataaaggctacaatgggttggcggaagtaggaaaaaagttcgagaaagacacaggaatcaaagttacggtcgagcaccccgataaactagaggaaaagtttccacaggtagctgctacgggggacggaccagacattatcttttgggcccacgatagattcgggggttatgctcagtccggacttctggccgagattactccagacaaggccttccaagacaaactttacccgttcacatgggacgcagtcaggtacaatggaaagctgattgcatatccgatagctgtggaggcacttagcctaatttacaacaaggatctactacctaaccctcccaagacttgggaagaaattccagctctggacaaggagttaaaagcaaagggtaagagtgcacttatgttcaatctacaagagccttatttcacatggcccctaatagccgccgacggaggctatgcctttaagtacgaaaacggcaagtatgacataaaggatgttggggtagacaacgcgggagccaaggctggattaactttcctggtggatttaattaagaacaaacacatgaacgcagacactgactactctatcgcagaagcagcgttcaataaaggcgaaacggcgatgacaattaacgggccctgggcttggtcaaacattgacacgagtaaagttaactatggtgtaacggtattgcccacatttaagggacaacccagtaaacctttcgtaggagtcttgtcagccgggatcaatgcagcttccccgaataaagagcttgctaaggaatttcttgaaaattatcttttaaccgatgagggattggaggcggttaacaaggacaagcctcttggtgctgtagccctgaaatcctatgaagaagagttagctaaggacccaagaatcgccgcaacaatggagaatgctcagaagggagaaattatgccaaatataccacaaatgagtgccttctggtatgcggtaaggacggcagttattaatgccgcttcaggtagacaaacagtcgatgaggctttgaaagatgcacagactaacagttcatccaacaataataacaataacaataacaataacctgggtatcgagggccgt

SEQ ID NO: 168 - protein, 630 aa, synthetic construct
MSSSHRILCVAFCVCSLVAVSSIYSPPFNHPIVYSNNAICFQLSTWRIPQLYLAVLIRRFDLSLSTRGVMLAVTVGVGSSQHHSQRSGLSLTFAMTPSMITLLVKTCVISQRTAVGEPHRPGWVWEVGRTEDETCHAHSPRGAPCHQGRIPLYSAAVESVDQIQVAVRFAQRHRLRLVVRNTGHDTAGRSSGSDSFQIHCHRMKQIEYHDNFRALGSDIDRGPAVSVGAGVTLGEMYARGARDGWVVVGGECPTVGAAGGFLQGGGVSSFHSFIDGLAVDNVLEFEVVTAKGDVVVANDHQNPDIFWALRGGGGGTFGIVTRATMRVHLNSPVCVSEVAVSGLRNNSLLWTKGITGLFSILRSFNQQGIPGQFILRPLSKDQVNASLTLYSLNTDDTRRSAENMLSIRNILESTTLPFTLASRCLPKISDALRKGPDMLPVNYGIITGSVLVSEDLFNSEEGPLHLAKQLEHFPMGPMDLLFTSNLGGNVSANTGKKHRDTSMHPGWRQAAHLINFVRSVSTPTAHEKARSLEELHSVQMRQLYDIEPDFRVSYRNLGDPLESDAAQVYWGPNYKRLLEIKRKWDPEDLFFSQLGVGSEGWTEDQMCKRQQRLQQMLQYLMSSIAQRVYR

SEQ ID NO: 169 - protein, 623 aa, synthetic construct
MSSGQSRGILGGVRQLILVILVGAYLSRLSAVDDDRHDCRCRPGEPCWPTVSHWSTLNESIGGALAHVKPIAHVCHQSGQDSSACEQVLQESLDSKWRASHTGALQDWVWEGGAESNQTCYYLRSGPAGSCHQGRIPLYSAAVKSASDVQKVVDFTRQHNLRLVIRNTGHDGSGRSSGPDSVEIHTHHLNSVQYHPNFRPAGSSERQSAPGQPAVTVGAGILLGDLYARGASEGWIVVGGECPTVGAAGGFLQGGGVSSWLSYAHGLAVDNVLEYEVVTAKGEIVIANAHQNPDLFWALRGGGGGTFGVVTQATLQVHPDLPVSVADAVVTGSRADATFWSHGVAALLRALQFLNNHGTAGQFILRNTDDDTVQASLTMYFSNLTVPAVADERMDPLRRALEHNGHPYQLTSRFLPQISSTFRHTADRYPEDYGILMGSVLVSLDLFNSATGPAALAQHFARLPMTPDDLLFTSNLGGRVSSLNRDPASTAMHPGWRDAAQLLNFVRGVGAPSLAAKATALHELQTVQMARLYEIEPAFQISYRNLGDPSERRSREVYWGSNYARLVEVKRRWDPEGLFFSKLGIDGDAWDAEGMCRQTRQGAWNVAVKWVQSFLGSLSASVV

SEQ ID NO: 170 - protein, 597 aa, synthetic construct
MSSYYLLGPVACFALIASVLWAPSGRCRCRPWEPCWPSAADWHVLNDSLQGSLVRIRPVASVCHGSEYDVAACANLSSLVVDSGWRASNPNTLQDWVWEIGNGVEEFCPYTISPMERRQSICPQGRLPFYSAVVRSTSDIQASVRFASRHNLRLVIKNTGHDSAGRSSAPHSFQIHTSLLQNISLHKNFIARGSTTGRGPAVTLGAGVMQWQAYVHGAKNGYTILGGECPTVGAIGGFLQGGGVSSIHSFTRGLAVDQVLEYQVVSAKGDLITANEDNNQDLFWALKGGGGGTFGVVTEATVRVFSDDPVTVTSTKIEAAAANVLFWKEGVHELLRLLQRFNNLHVAGQLVISAPTKDSLQAGLELHFANLTDETQAIQLLRSEARALETHGISASTSVRVQRKASSELRMKPDLYPPHYGILEASVLISAATFHANDGPALIASKLSGLTLKPNDILFTSNLGGRVSENTAIEIALHPAWREAAQLVTLVRVVEPSIEGKLSALNDLTARDVPILYSIDPAAKISYRNLGDPQEKEFQARYWGADNYARLAATKAAWDPSHLFMTSLGVGSEVWDAEGICRKRRGFRAKASSLIGM

SEQ ID NO: 171 - protein, 627 aa, synthetic construct
MSSALHLALFLTAAALLVAFAQSQTLFGYEKTQLTRKYVASLPQEDARLFAFEDQFEDRFEATAVSATECKVNPTDKKWPAERAWKRLESRLSSPNNLIAAVPQASVCYAPTRDDAQCQQLTRNWTNSYTHIDDPTEVLSPIYQGLTCQPPLIFDSGNCTIGGYPAYVVKATAVSDIQTAINFARNDNLRLIVKNTGHDFAGHSVGYGALSIWTHVLKDMMFFDNYVDESGYKGPAIKAGAGVQAFELYKFANEHGVVAVAGEGQTVGVMGGFLLGGGHSPLSSIYGMASDHVLGFEVVTPIGEFLTANSTSNTDLFWALKGGGGGTFGVVTSVTFKVYPDMPVTTVSWTLDSSKIGKDKFWAATKAFFDRAIDNVDAETYSYNSIIPIGENFLSIMQPFFAPNKTATQLNTILGPYFSKLTALNIPFSPKTTEYKTFYPAWQAEFPLEPMSGTNSATASRLFPRSNFVSDIGKTTTFAVLRQTLESGKPLIAFNMAPRLLPSNPDNAINPAWRTSVYHIITGATIDPRSSPATQLAARAALTNGTMRAWRDVTPGSGAYLNEADRLEPDWQRSFWGANYERLVGIKREMDRRDVFWVDRGVGSEGWRVESVDGVPNENGPLCRVGG

SEQ ID NO: 172 - protein, 602 aa, synthetic construct
MSSYNLLGPVACLTLAGLVLWLHSGHCRCRPWEPCWPSAAHWKAFNESIQGNLVRIRPVASVCHTLEYDAVACANLSSLVVDSGWRASNPSTLQDWVWESGNTAEETCHSMTSPGEQRRSVCHQGRLPLYSAVVQSASNIQSSVRFAADHNLRLVVKNTGHDCAGRSSSPHSFQIHTNLLKNISFHENFVAGGSTTGYGPAVTLGAGVMHWEVYAHGAEKGYTILGGECPTVGAVGGFLQGGGVSSIHSFTRGLAVDNVLEYQVVTSNADLVTANEHENQDLYWALKGGGGGTFGIVTQATVRVYPDDPVTVTTTVIKAAFTNTLFWTEGVRELLRLLRDFNLLHIPGQLVVRGPEKHSIQATLELHFANMTDEAYAMKLLSSELRPLSIHHISTSTSARVQKRASSELRMKPDIYPPHYGILQGSVLISTAVFQSAEGPTLLARKLSELTMRFSDILFTSNLGGRVSKNIAIDIPLHPAWREAAQLVTLVRAVEPSTKGKLSTLENLTSRDTPILYSIDPSAKISYRNLGDPQEQEFQAKYWGADNYARLAAIKAKWDPNELFITRSGVGSENWDVEGICRKSQDFIAQAFALLKVGLSSH

SEQ ID NO: 173 - protein, 622 aa, synthetic construct
MSSYQLLAPAAALALGVAVFRSQPNQHQCRCRPSEPCWPSADQWDVFNKSIGGNLARIRPVASVCHGPEYDAAACTALSSLARDSGWRASNPATLQDWVWEGNGLAEEICHVVTSPEESQQSACHQGRVPLYSATVQSTTQIQSSVRFAKKHNLRLVVKNSGHDCAGRSSSPDSFQIHTNLLKGITYHDNFVAQGSAANGSGPAVTMGAGIMHWEVYSDGAKNGYTVVGGECPTVGAVGGFLQGGGVSSFHSFTKGLAVDNVLEYQVVTASGELIAANEYQNRDLFWALKGGGGGTFGVVAQATVRVYPDDPITVAAISISTSLTNSLFWTEGVSELLRLLRIFNQQGFPGQIVVMGPNQGSVQATLEFHFANTTDESYVNGLLNSQMKSLGDHKISTNLSIRVQKKASSEVRLGPDTYPPQYGIVQGSVLISAALFNSADGPPLIARNFSNLNLGPNDILFTSNLGGRVMDNSDIDIPLHPSWRSAAQLVTLVRAVEPSNKGRLAALETLHSHDMPILYSLEPKNKVSYRNLGDPSEREFQSRYWGSNYKRLASIKERWDPDELFITRLGVGSEEWDTEGMCRKSIGLVARVSELLRLSGRWILGGFSALIQQVDIIDSSR

SEQ ID NO: 174 - protein, 606 aa, synthetic construct
MSSSCRCQPWEPCWPSDEQWELLNRSMEFTLVRLQPIASVCYNHSLDHTLCEDVMRMSRDSGWRASQPGALQDWVWESGQSPGDACPLGSQMNGHTQCHQGRIPLYSAMVNSTKHVQEAVMFAKRHDLRLIIRNTGHDLAGRSSSPNALQIHTHRLQDIKFHDNVQLHGFEKSFGPAVSVGAGVMMGDLYARSAQNGYIVVGGDCPTVGVVGGFLQGGGISDFLSLHHGLAVDNVLEFEVVTASVSLPEPDAIQYCGFLTLRQGDIVLANAIRNPDLFWALRGGGGGTFGIVTRATMRVFPDVPAIAAELGVQTSQSHGEYSRSLAAFFTVLQSLNRENVGGQLIITVISEHSIEAKLKMFFLNQTNTADVDQRMQPFVEDMRRIETHVTYESTSLPKLSMNYRQVPDIHTDNDYGVLGSTVAISNDLFNASKGPAYVADGLAQLPTRPGDLLFTSNLGGQVMRNGDLMETSMHPAWRNATQLINFVRPVEPTIEGKAGALQNLTNIHMPLLYAIDPRFRLSYRNVGDPNEKDFQQVYWGQSYARLLQLKRRWDREGLLISKLGVGSEEWDSEGMCRTGRRSLENLAIDTLSSLAHLIHVTYRCIW

SEQ ID NO: 175 - protein, 602 aa, synthetic construct
MSSHRLLGPVACITLAGAVFWAQSGHCRCRPWEPCWPSTAQWEAFNESIQGNLVRIRPIASVCHGLEYDAIACANLSNLIVDSGWRASNPSTLQDWVWESGNVAEEACHIMTSPGEQRGSACHQGRLPLYSAVVQSTSDIQSSVRFASDHNLRLVVKNTGHDCAGRSSSPNSFQIHTNLLKSISFHENFVARGSTTCSGPAVTLGAGVMHWEVYAHGVENGYTIVGGECPTVGAVGGFLQGGGVSSIHSFTKGLAVDNVLEYQVVTPNADLVTANEHENQDLYWALKGGGGGTFGIVTQATVRVYPDDPVVVTATMIKTAFANTLFWTEGVRELLRLLQYFNQLHIPGQLVVTGPEKDSIQATLELHFANMTDEAHAMKLLSSELRPLSIHHISTSTSVRVQKRASSELRMKPDIYPPQYGILQGSVLISTAVFQSAEGPTLIARKFSELTVRSNDILFTSNLGGRVSKNIAIDVPLHPAWREAAQLVTFVRAVGPSIEGKLSALENLTSQDIPILYSVDPNAKISYRNLGDPQEQEFQARYWGLDNYARLAAIKAKWDPNGLFITRSGVGSEDWDVEGICRKSRGLIAQALELLKLGPYIY

SEQ ID NO: 176 - protein, 545 aa, synthetic construct
MSSEDGGCKCFPGNACWPGHSDWDRLNSTVGGRLIATTPLGSPCHDPHYNETECATLQKEWLYSAVHMESSSSVMAPLFANKSCDPFQPRSSPCTLGNYVRYAVDARSPQDVAATLTFASRHRIRLVVRNTGHDYNGRSTGAGALAVWTHHMKDSAVIDWADRHYIGKALRVGAGVQGFEALAEARKHGRVVVTGECPTVGLAGGYTQGGGHSALSTVHGLAADNVLSYEVVTASGKHVRASRAENSDLYWALSGGGGGNYGVVVSMTVRAHPDAKVSGASFAITAPENSTSLYDAIDLFHEALPGMVDSGAMVIYYFGAGFLQMPALTLFGKSTEETEKVLKPFIDAASAQGFVVKPNYTEFESYYDHYAHYWGPLPAGNIQVGTQLFGGRLIPRDALTHFSPTARELVSMGVTFIGVGLDVSRFGREKAAAVLPQWRQTLVHASLTMPWSFDAPFEEMVAEQNRMTREVQPVIEAATPGAGAYMNEADFQQPDFQETFFGANYDRLLRIKKKYDPNGLFYATAGVGSEEWTVKKDGRLCRAHR

SEQ ID NO: 177 - protein, 577 aa, synthetic construct
MSSRSTAYLTALLSFLGATYAAPEGPRCRCTPDQSCWPSPGLWQTLNKTLSGNLVAVKPVGAVCHDPTYNGGLCDSVKGAVQSINWETWPEKNESCYIDGPRQVPCGQGRIPLYSAVVQSPLDIQKTVRFASKYNLRLVIKNTGHDFLGRSTGPQSLQILTHNMKSINFTDNFVPEGKPDGRGIGQAVTIGAGVQLNELYEAAGKRGLTQVIGLSTTVGAAGGYIQGGGHSPLGPWKGMSTDHVLEYKVVTAGAKFVTANEYQNSDLFWALRGGGGGTFGVVTSVTLQTFKDPPTIVSQVNITMDGKANESYWASVEKFQAYLPTLSDGGCSGYYYMLPNVTLGPQSAAVIIAAFYYANKTDKAHVDNLYRPLFASLSSIPGINVASVSVPVSSSTEAFRSAFDQKPPRDGGGVNILGSRLFSRKLLETPGGAANLTTALSKLDFKNLQPAIGHLVAGGQVAKNTHIQSALNPSWRKALVHLVISRDWSIDSTFAEQEKISTKLTAEEIPLLAAVEPDMGAYTNEADVNEPRFQQTFWGTNYNTLLRVKNRWDPRGLFFVRSGVGSEAWDKQGLCRV

SEQ ID NO: 178 - protein, 606 aa, synthetic construct
MSSQFLLWSTGLVALLSWLIYTQETQSASCRCRPWESCWPSEELWNSFNTSVDGKLHRLRPAAHVCYGPSFNRSACDNILLLSRDSGWRASNPGVLQDWVWEAGETANESCPVGSLRTASAVNSCHQGRIPLFTVGVESTKQVQEAVRFARKHKLRLVIRNTGHDLAGRSSAPDSFQIHTHRLQEIQFHADMRLDGSNTSLGPAVTVGAGVMMGDLYAQAARHGYMVLGGDCPTVGVVGGFLQGGGISDFLSLNQGFGVDNVLEYEVVTADGELVVANALQNQDLFWALRGGGGGTFGVVTRATMRVFPDVPVVISEILLEAPQAISSSWTQGLSIVLTALQSLNHDNVGGQLVIAVLPNLAVQASIKFFFLDATEAAVIDRRMKPFLTKLSRANVKYTYSSKNLPHFSSNYRQVPDIHSDNDYGVLGSTVAISQQLFDSPQGPEKVAKALANLPVSAGDLIFTSNLGGRVIRNGELAETSMHPAWRSASQLINYVHTVEPSIEGRAKARERLTNTQMPMLYALDPNIKLSYRNVGDPNEKDFQQIYWGPNYGRLSNIKKKWDTDDLFFSKLGVGSERWDSEESDEVYFFSYFYLEGINTLKVARR

SEQ ID NO: 179 - protein, 475 aa, synthetic construct
MSSASEVSVASSGSEHSGAQKCPFQDPGLSSMDQDSRLRDILSRFNREKIPERAVHARGAGAYGEFEVTHDVSDICDIDMLLGVGKKTPCVVRFSTTTLERGSAESVRDVKGMAIKHFTQDGNWDWVCLNIPMFFIRDPSKFPDMVHAQRPDPTTNVANPSRWWEFVCNNHETLHMVMFQFSDFGTMFDYRSMSGYAAHAYKWVMPDGSWKYVHWFLASDQGPNFETGHQAKQIGADDAESATRDLYQSLERGEYPSWTVKVQVVDPEDAPKLPFNILDVTKHWNLGNYPPDIDVIPGRTLGKLTLKKGPQDYFEEIEQLAFSPSRLVHGVEASEDPMLQARLFAYPDAQKHRLGPNNLDLPANRTKKLADGSRPEKAEMAPQKVPSQEHADWVSQVKSSSWSEPNETDYKFPREFWKALPRLRGEAFQNSLVVNMAKSVSQVPADMRQKVYSTLALIADDLADRVRTMTEEIVE

SEQ ID NO: 180 - protein, 477 aa, synthetic construct
MSSPSNISVQTRGYGDSGSQSGQVNQAYSAIDYRLLDSLSRFNREKVPERAVHARGAGAYGEFEVTDDISDICDIDMLLGVGKKTPCVVRFSTTTLERGSAEAVRDVKGMAIKFFTQDGNWDWVCLNIPMFFIRDPSKFPDLIHAQRPDPRTNLTNPSKWWDFVCKNHETLHMVMFQFSDFGTMFDYRSMSGYINHAYKWVMPDGTWKYVHWFLASDQGPNFEKGEQAKVIAADDTESATRDLHQSLERGEYPTWTVNVQVVDPEDAPQLAFNILDVTKHWNLGNYPPDIPVIPSRRLGKLTLKQGPKSYFEEIEQLAFSPSHLVHGVEPSEDPMLQARLFAYPDAQRHRLGPQNLDIPANRSRHTVDGAKTKKTVPPEKKSPEHAEWVSQVTTSSWSQPYEADYKFPREFWKALPRLRGQEFQDNLVVNMSKSVSQIPVDMRQNVYNTLGLVAEDLADRVRIMTEEMMDSEKGVPL

SEQ ID NO: 181 - protein, 522 aa, synthetic construct
MAALIERGLLSIMASKCSGTWSTYKTYTTANGCPFMKPEGPPADGRTLALNDHHLVESLAHFNREKIPERAVHAKGAAAYGEFEVTADISDICNIDMLLGVGKKTPCVTRFSTTGLERGSAEGMRDLKGMATKFYTKEGNWDWVCLNFPFFFIRDPLKFPSLMHAQRRDPRTNLLNPNMYWDWVTSNHESLHMVLLQFSDFGTMFNWRSLSGYMGHAYKWVMPNGSFKYVHIFLSSDRGPNFSQGEQAKDNSDLDPDHATRDLYEAIERGDYPTWTANVQVVDPAEAPDLGFNILDVTKHWNLGTYPKDLPKIPSRPFGKLTLNRIPDNFFAEVEQLAFSPSNMVPGVLPSEDPILQARMFAYPDAQRYRLGPNHHKIPVNQCPMTFNPTLRDGTGTFDANYGSLPGYVSESQGVNFARPQEHDPKFNAWLSQLSSRPWMQTNENDYKFPRDFYNALPEFRSQEFQDKMVENIIASVAQTRREIREKVYHTFHLVDPELSARVKRGVEKMDASFKQVSLSRL

SEQ ID NO: 182 - protein, 468 aa, synthetic construct
MAAAPNYAKKCPVMGKAPSSGHSSIGPQDVHTLEVLSNFNREKIPERVVHALGAGAYGEFEVTHDISDICNIDMLLGVGKKTSLVTRFSTTGLERGSSEGVQDLKGMATKLFTSDGEWDWVFLNFPFFFIRDPVKFPDMIHSQRRDPQTNRLNPNNFWEFVTENHESIHMVLLQYSDFGRMFTWRTLSSYSGHAFKWVMPDGSFKYVHFFLSSDRGPNFKDGAGTIMDSSEPDFASRDLFEAIERGDHPSWTANVQVIDPKDAPHLGFNILDVTKHWNLGTYPQGVEKIPSRPFGKLTLNRNVKDYFSEVEQLAFSPSHLVPGIEASEDPILQARLFAYPDAQRYRLGRTLSKQASPRTSSTAEYQASLGKDFAEWVAQVSSDVWSQPHEDDYKFAREYYEILPEFRSQEFQDRMVERIVESVSQTRQDIRNKVYRTFALVSSELATRVQEGVEGAEIGQEKNVQARL

SEQ ID NO: 183 - protein, 478 aa, synthetic construct
MSSASKTSVETNESGDSRSQRGRGYQAYSAIDHRLLDSLSRFNREKIPERAVHARGAGAYGEFEVTDDISDICDIDMLLGIGKKTPCVVRFSTTTLERGSAEAVRDVKGMAIKLFTQDGNWDWVCLNIPMFFIRDPSKFPDLIHAQRPDPKTNLTNPSKWWEFVCNNHETLHMVMFQFSDFGTMFDYRSMSGYVSHAYKWVMPDGSWKYVHWFLASDQGPNFEKGKQTKEIAPNDPESATRDLHQSLDRGEYPTWTVNVQVVDPEDAHQLAFNILDVTKHWNLGNYPPDIPVIPSRRFGKLTLKQGPKDYFEEIEQLAFSPSHLVHGVEPSEDPMLQARLFAYPDAQRHRLGPQNLDMPANRTRHTADGAKTKKVVPPEKKSREHAEWVSQMTSSSWSQPDEADYKFPREFWKALPRLRGEEFQNNLVVNMSKSVSQIPVDLRQKVYNTLGLVAEDLADRVKAMTEEMIMESEQRARL

SEQ ID NO: 184 - protein, 602 aa, synthetic construct
MAAHRLLGPVACITLAGAVFWAQSGHCRCRPWEPCWPSTAQWEAFNESIQGNLVRIRPIASVCHGLEYDAIACANLSNLIVDSGWRASNPSTLQDWVWESGNVAEEACHIMTSPGEQRGSACHQGRLPLYSAVVQSTSDIQSSVRFASDHNLRLVVKNTGHDCAGRSSSPNSFQIHTNLLKSISFHENFVARGSTTCSGPAVTLGAGVMHWEVYAHGVENGYTIVGGECPTVGAVGGFLQGGGVSSIHSFTKGLAVDNVLEYQVVTPNADLVTANEHENQDLYWALKGGGGGTFGIVTQATVRVYPDDPVVVTATMIKTAFANTLFWTEGVRELLRLLQYFNQLHIPGQLVVTGPEKDSIQATLELHFANMTDEAHAMKLLSSELRPLSIHHISTSTSVRVQKRASSELRMKPDIYPPQYGILQGSVLISTAVFQSAEGPTLIARKFSELTVRSNDILFTSNLGGRVSKNIAIDVPLHPAWREAAQLVTFVRAVGPSIEGKLSALENLTSQDIPILYSVDPNAKISYRNLGDPQEQEFQARYWGLDNYARLAAIKAKWDPNGLFITRSGVGSEDWDVEGICRKSRGLIAQALELLKLGPYIY

SEQ ID NO: 185 - protein, 498 aa, synthetic construct
MSSGANDAPASTYRYDEKPVYTTSNGAPVNNPSGWQRPGPMGPLLLQDFHLIDQLAHFDRERIPERVVHAKGAGAYGEFEVTHDVTDITSIDMFSKVGKKTKCVTRFSTVGGEKGSPDSARDPRGFSVKFYTEEGNWDWVYNNTPIFFLRDPTNFPLFIHTQKRNPQTNLKDATMFWDYLSTHQEAIHQVMHLFSDRGTPYSYRHMNGYSGHTHKWTKPDGSFVYTQVHLKTDQGNKTFTNEEAGKLASENPDWHTQDLFESIQKGDYPSWTVYVQVLTPEQAEKFRWNIFDLTKVWPQKEVPLRPVGKLTLNKNVENYFAEIEQVAFSPSHLVPGVEPSADPVLQSRLFSYPDTHRHRLGTNYQQIPVNAPLHPFNPYQRDGAMVVNGNYGANPNYPSSYRPLTYKTAPPVNTHEQWSGSAVHALFQEINDDDYVQAKGLWDVLGRTSGQQDNFVGNVAGHLSAAHQDTRNRTYDMFSRVDAGLGKAIKEQTEKQVK

SEQ ID NO: 186 - protein, 480 aa, synthetic construct
MSSAPNAADKCPVMGNSGEKCPVMSSSTQSRGPRDIYTLEALSHFNREKIPERAVHAKGTGAYGEFEVTADISDICNIDMLLGVGKKTQCVTRFSTTGLERGSSDGVRDLKGMAVKFFTEQGDWDWVSLNFPFFFIRDPAKFPDMIHSQRRDPQTNLLNPNMTWDFVTKNPEALHMTLLQHSDFGTMFTWRTLSSYVGHAFKWVMPDGSFKYVHFFLASDRGPNFTDGSTAKVDPNDPDFATKDLFEAIERGDYPSWTANVQVVDPKDAPKLGFNILDLTKHWNLGTYPKGLDTIPSRPFGKLTLNRNVKDYFSEVEKLAFSPSNLVPGVEPSEDPILQARMFAYPDAQRYRLGIDHLKAPLRRKETACQHDLGPEFEKWLSQVTSEAWSHPHEDDYKFAREYYEVLPEFRSQEFQDRMVENLCKSIAPGPEELRKRVYDTFELVSSELARRLREGAEAIVAEKARPDSPSRAQPGQLRL

SEQ ID NO: 187 - protein, 263 aa, synthetic construct
MSSASVESRIIAITGGASGIGAATCRLLAERGAAVLCVCDISPKNFDDLKISIKKINPSTKVHCATVDVTSSVEVRQWIEGIISDFGDLHGAVNAAGIAQGAGMRNTPTIAEEVDEEWTRIMNTNLNGVFYCTREEVRAMKGLPATDRSIVNVGSIASVSHMPDVYAYGTSKGACAYFTTCVAADAFPLGIRINNVSPGVTNTPMLPQFAPMAKTFEEIEESYKKEGLSLIEAEDVARTIVWLLSEDSRPVFGANINVGACMP

SEQ ID NO: 188 - protein, 264 aa, synthetic construct
MSSTVVNSRIFAVTGGASGIGAATCRLLAARGAAVICIGDISPKGFESVTSDIQALNPATKVHCTRLDVSSSAEVDAWLQTIVQTFGDLHGLANVAGIAQPAGARQAPALLEETDEEWQRVFKVNLDGVFYCTRAGIRAMKALPGPKDRAIVNIGSIAAFIHLPDAYAYGTSKGACAYFTSCAAMDAWPLGIRINSVSPGITDTPALPQFMPQAKSIDEVKEAYIKEGFPIIQPEDVARTIVWLLSEDSRPVYGANINVGAATP

SEQ ID NO: 189 - protein, 263 aa, synthetic construct
MSSASVSSGIFAVTGGASGIGAATCRLLADRGAAIICLADVSNTNFVSLQREIAEINPSTFTQCTVLDVTSPQDVNKWLQDIISTHGDLHGAANVAGIAQGAGLRASPTILEENDSEWRRILDVNLNGILYSTRAQVRVMKELTHGNRSIVNVASIAAFSHVPDVYAYSTSKNACAYLTTCIAADVVSLGIRVNCVSPGITNTPLLPQFEPKAKSLSEIQEIYLRQGLPIHEAEDVARTIVWLLSDDSKPVYGANINVGACAP

SEQ ID NO: 190 - protein, 263 aa, synthetic construct
MSSPSMTSKVFAITGGASGIGAATCRLLAERDAAVICLADVSSTNFTSLQESIAKSNPSTLVHCTELDVRSADKVDQWLQSIVSTHGDLHGAANVAGIAQGAGLRATPTILEENDAEWSRILDVNLNGVFYSTRAQVRVMKDLPPGHRSIVNVASIAAFSHVPDVYAYGTSKSACAYLTTCIAADVFWSGIRVNCVSPGITNTPMLPQFEPKAKSLDAIKDMYRDQGYPTGEADGVARTIVWLLSEDSIPVYGANINVGACPP

SEQ ID NO: 191 - protein, 263 aa, synthetic construct
MAASSVSSKIFAITGGASGIGAATCRLLADRGAAVICLADVSSNNFVSLQESIAKVNSSTLVQCTALDVSMANEVNQWLQDIISIHGDLHGAANVAGIAQGAGLRARPTILEEKDDDWSRILDVNLNGVFYSTRAQVRAMKDLPRGHRSIVNVASISVFSHVPDVYAYSTSKNACAYLTTCIAADVFWSGIRVNCVSPGITNTPMLPDFEPKAKSLDEIYEIYRGQGYLTCEAEDVARTIVWLLSEDSRPVYGANINVGACPP

SEQ ID NO: 192 - protein, 263 aa, synthetic construct
MSSTSVTSKVFAITGGASGIGAATCRLLSKRGASVICVGDIVNDHFDELRRDIARINNTTKVSCTVLDVTSSAQIERWISYIVDTYGDLYGAANIAGIAQGADLRTSPTILEETDDEWSKVMKVNLDGVFYCTRAEIRGMKNLERIGRSIVNVGSIAAFLHQPDVYAYGTSKAACAYMSTCAAADAFPFRIRVNTVSPGITNTPLLPSFNPKLNSLDEVKELYAKNGLPLIEPEDVARTIVWLLSDDSWPVYGSHINVGACVP

SEQ ID NO: 193 - protein, 263 aa, synthetic construct
MSSASLTAKIFAVTGGASGIGAATCRLLAERGAGVICVADVSSTNFGSLQKSITEINSLTLVQCTVLDVTSSEDVNQWLRDIVSSHGNLHGAANIAGVAQGAGLRTTPTILAETDADWSRILKVNLDGVFYCTRAQVRAMTDLPKGNRSIVNVASIAAFSHMPDVYAYSTSKQACVHFTACVAADVFPLGIRVNCVSPGITNTPLLPQFEPNAKSLDEIQALYASQGLTMCGADEVARTIVWLLSEDSHPVYGANINVGAGVP

SEQ ID NO: 194 - protein, 263 aa, synthetic construct
MSSASVKSRVFAITGGASGIGAATSRLLAERGATAVCVGDISCKNFDELKESMKKINPATEVHCSLLDVTSPTEVEKWVKSIVAKFGNLHGAANIAGIAQGAGLRNSPTILEEGDEEWSKVLKVNLDGVFYCTRAEVRAMKSLPSDDRSIVNVGSIAALSHIPDVYAYGTSKGASTYFTTCVAADTFPFGIRVNSVSPGITDTPLLPQFIPKAKTSDEVKEVYRKEGFSVVKAGDVARTIVWLLSEDSSPVYGANINVGASMP

SEQ ID NO: 195 - protein, 263 aa, synthetic construct
MAASSVSSKIFAITGGASGIGAATCRLLADRGAAIICLADVSSNNFVSLQESIAEVNPSTLVQCTVLDVSSAVDVNQWLQDIVSIHGDLHGAANVAGIAQGAGLRATPTILEEKDADWSRILDVNLNGVFYSTRAQVRAMKNLPCGNRSIVNVASISAFSHVPDVYAYSTSKNACAYLTTCIAADVFWSGIRVNCVSPGITNTPLLPQFEPKAKSLNEIQEIYRRQGYSTCEAEDVARTIVWLLSDDSRPVYGANINVGACPP

SEQ ID NO: 196 - protein, 267 aa, synthetic construct
MSSSFVSSKIFAITGGASGIGAATCRLLAKRGAATLCVGDLCSENMKLLEKDIKKINPDTKVHCTVLDVSSSSNVDEWIQDIITTFGDLHGAANIAGIAQGAGLRQAPTILEDDDQQWKKVFQVNLDGVLYSTRAQVRAMKEFSSTNPGDRSIVNVASIASMSHMPDVFAYGTSKAGCAYFTTFEVTLFALGYFSNIMGMLEGITRTPMLPRFVPSAKTQEEVEETYKKEGFSVIEADDVARTIVWLLSEDSRPVFGANINVGACMP

SEQ ID NO: 197 - protein, 378 aa, synthetic construct
MSSREEPSSAQLFKPLKVGRCHLQHRMIMAPTTRFRADGQGVPLPFVQEYYGQRASVPGTLLITEATDITPKAMGYKHVPGIWSEPQREAWREIVSRVHSKKCFIFCQLWATGRAADPDVLADMKDLISSSAVPVEEKGPLPRALTEDEIQQCIADFAQAARNAINAGFDGVEIHGANGYLIDQFTQKSCNHRQDRWGGSIENRARFAVEVTRAVIEAVGADRVGVKLSPYSQYLGMGTMDELVPQFEYLIAQMRRLDVAYLHLANSRWLDEEKPHPDPNHEVFVRVWGQSSPILLAGGYDAASAEKVTEQMAAATYTNVAIAFGRYFISTPDLPFRVMAGIQLQKYDRASFYSTLSREGYLDYPFSAEYMALHNFPV

SEQ ID NO: 198 - protein, 377 aa, synthetic construct
MSSSTSKLFKAFPLGTNVLQHKVVLSPMTRFRADENGVPLPYVKTFYAQRACIPGTLLITEAVAISPRAKGFQNVPGIWHPDQVSGWRDVVDEVHSKGAVIWLQLWATGRAADQAALASNGLELESSSEVPVAPGEPTPRALSEEDIESYIFDYVQGAKNAIHGAGFDGVEIHGANGFLIDQFLQSSCNRRTDQWGGSIENRSRFGLEITRQVINAVGKDRVGMKLSPWSTFQGMGTMDDLVPQFQNFIAELREMGIAYLHLANSRWVEEEDPTIKTHPDVNNETFVRMWGEKRPILLAGGYGPDSARHVVDQTYAEQNNVLVVFGRHYISNPDLPFRLKMGIALQKYNRDTFYIPASDKGYLDYPFCEEYLKYVGK

SEQ ID NO: 199 - protein, 382 aa, synthetic construct
MSSSTSNLFSTVPFGKNVLNHKIVLSPMTRFRADDNGVPLSYMKTFYAQRASVRGTLLVTDAVAICPRTKGFPNVPGIWHKDRIAAWKEVVDEVHSKGSFIWLQLWATGRAADLEALTSRGLKLGSSSEVPVAPGEPTPRALDEDEIQQYILDYVQGAKNAVHGAGFDGVEIHGANGFLIDQFLQSSCNRRTDQWGGSIENRSRFGLEITRGVVDAVGHDRVGMKLSPWSTFQGMGTMDDLVPQFEHFITCLREMDIAYLHLANSRWVEEEDPSIRTHPDFHNQTFVQMWGKKRPILLAGGYDPDSARRLVDQTYSDRNNVLVVFGRHYISNPDLPFRLRMGIALQKYNRDTFYIPCSGEGYVDYPFCKEYLDQADEAAVAG

SEQ ID NO: 200 - protein, 386 aa, synthetic construct
MSSSTSNLFNALPFGRNVLEHKIVLSPMTRFRADNSGVPLPYVKTYYAQRASVRGTLLITEAVAICPRAKGFPNIPGIWNDSQIAAWKEVVDEVHSKGSVIWLQLWATGRAADTDTLTSNGWELESSSEVPVAPGGPTPRSLTEEEIQSYIVDYAQAARNAVHEAGFDGVEIHGANGFLVDQFLQSSCNRRTDQWGGSIENRSRFGLEITRRVIDAVGNDRVGVKLSPWSTFQGMGTMGDLVLQFEHFITNLREMDIAYLHLANSRWIEEEDPSIETHPDIHNETFVHIWGEKRPILLAGGYDSDSAQRLVDQTYSQQNNVMVVFGRHYISNPDLPFRLKMGIALQRYNRDSFYIKFSGEGYLDYPFCKEYLEQEDEDTTASMRKN

SEQ ID NO: 201 - protein, 369 aa, synthetic construct
MSSSNQSGLFCPLQLGKCYLKNRVILCPLTRFRADVGAVPSSLMKEYYTQRASVPGTLLITEATSISEDARGFDKVPGIWNQDQIAAWKTIVDAVHSKNSFIWLQLWATGRAAEPGFNCVSSSSIQIPPQEDPNEIYPVPRSLTSMEVENFINQYVLAAENAMNCGFDGVEIHGANGFLIDQFTQESCNKRTDEWGGSLEKRARFGLEVTKRIISKIGADRVGIKLSPWSTYLGMGVMEDLVPQFEYIIKQLHEMNVAYLHLANSRWVESKSHPDVDNETFVRMWGKAKPIIIAGGYNGESARKVVEDTYAGHNNIAVGFGRHFLANPDLPFRLRHGIQLAPYDRETFYGPGSEGYTDYPFSSEFIDSH

SEQ ID NO: 202 - protein, 380 aa, synthetic construct
MSSSTSNLFKSIAFGKGVLQHKIVLSPMTRFRADDDGVPLPYVKTYYSQRACLPGTLLITEAVAISPRAKGFPNVPGIWSPEQIASWKEVVDAVHSKGSYIWLQLWATGRAADVDTLKANGFEIVSSSATPVGPDEPSPRALTEDEITSYIADYVQGAKNAVFGAGFDGVEIHGANGFLIDQFLQSSCNQRTDGWGGSIEKRSRFGLEITKQVIDAIGRDRVGMKLSTWSTFQGMGTMEDLVPQFHYFLSHLAEMDIAYVHLANSRWVEEEDPTIRTHPDIHNETFVRMWGDKKPILLAGGYDAESAQRVVDETYAKQDNVLVVFGRHYISNPDLPFRLKMSIDLQKYNRDTFYIPFSDEGYLDYPYSEEYLAQEQGATA

SEQ ID NO: 203 - protein, 382 aa, synthetic construct
MSSSTSNLFTPLQFGKCLLQHKLVLSPMTRFRADNEGVPLPYVKTYYCQRASLPGTLLLTEATAISRRARGFPNVPGIWSQEQIAGWKEVVDAVHAKGSYIWLQLWATGRAAEVGVLKANGFDLVSSSAVPVSPGEPTPRALSDDEINSYIGDFVQAAKNAVLEAGFDGVELHGANGFLIDQFLQSPCNQRTDQWGGCIENRSRFGLEITRRVIDAVGKDHVGMKLSTWSTFQGMGTMDDLIPQFEHFIMRLREIGIAYLHLANSRWVEEEDPTIRTHPDIHNETFVRMWGKEKPVLLAGGYGPESAKLVVDETYSDHKNIGVVFGRHYISNPDLPFRLKMGLPLQKYNRETFYIPFSDEGYLDYPYSEEYITENKKQAVLA

SEQ ID NO: 204 - protein, 377 aa, synthetic construct
MSSTIIPKHPSPTLFKPLALGKCQLQHRIVMSPTTRYRADEAAVPLPFVKEYYAQRASDPGALLITEATNICPNSVGEAHIPGIWSKTQCEAWREVVSQVHAKECYIFCQIYATGRSADPELLASRGFEQVSSSAVAAEPGCQPPRALDEEEIQKYISDYAQAARNAIEVGFDGVEIHGANGYLIDQFTQASCNQRTDEWGGDIPNRARFALQVTMAVINAIGPDRVGMKLSPWSQYSGMGIMGDLVPQFEYLILQLRQLGIAYLHLANSRWLDQMTTHPDPNHLTFVKVWGRSLPVILAGGYDATSAPEVIEMVYADYDNVAIGFGRYFTSTPDLPFRMKNGIALQKYDRSSFYTCLTKTGYLDYPYSPEYLCRSS

SEQ ID NO: 205 - protein, 382 aa, synthetic construct
MAASTSNLFNELPFGTSVLKHKIVLSPMTRFRADNGGVPLPYVKTYYAQRASVHGTLLITEAVAICPRAKGFPNIPGIWHDSQIAAWKGVVDEVHSKGSVIWLQLWATGRAVDPDTLTSNGLELKSSSEVPVAPGEPTPRPLSEEEIQSYIFDYAQGAKNAVHEAGFDGVEIHGANGFLIDQFLQSSCNRRTDQWGGSIENRSRFGLEITRRVIDAVGNDHVGVKLSPWSTFQGMGTMDDLVPQFEHFITRLREMDIAYLHLANSRWVEEEDPSIKTHPDVHNETFVRIWGEKRPILLAGGYNPDSARRLVDQTYSEQNNVMVVFGRHYISNPDLPFRLKMGIALQRYNRDTFYIPFSGEGYLDYPFCKEYLKQGDQETTVA

SEQ ID NO: 206 - protein, 286 aa, synthetic construct
MSSTILVLGGRGKTASRLSALLHEAQIPFVVGTSSTAPIATYRTAHFDWLNKDTWTNAWDQAPEPITAIYLVGAHAPEFVHPMFEFIDLAHSRGVRRYALVSASNTEKGDPMMGEAHAHLDSKEGVKYVVLRPTWFMENHIEDPHLSWIKTENKIYSATGPGKIPFVSADDIARVAFHALTQPLTQKEQQYMVLGPDLLSYADMAEILTDLLGRKITHVTLPAEKFGQFLVDTAGLPPDFAEMLATMEAAVATGAEERVSQDVEKVTGRPPRRFVDYARENREHWL

SEQ ID NO: 207 - protein, 290 aa, synthetic construct
MSSTILLTGGSGKTASRIAHLLKKANLPFVIGSRSSSPQTEKYHRSFNWLDETTFQNVLSSERGTQPISAVWLVPPPILDLAPPVNNFIDYACSQGVKRYVLLSASMIEKGGPAMGQIHEHLDSLESISYTVLRPTWFMENFSTPGEFQCESIRNEAKVYSAAGDGKVPFISAADIARVAFRALTGKLQDRTDHILLGPELLAYDDVAETLSKVINRKITHARVTESELSEKLQARGMSAEEAEMHSSLDLTIKSGAAADMNTVVRDLTGSEPCNFLEFAKREKKVWLSN

SEQ ID NO: 208 - protein, 292 aa, synthetic construct
MSSTVLLTGGTGRTAKHIAGIFRQTNVPFLVASRSSSAGTAENHRKFDWLDEETFPNALSVDQGMKPISVVWLCPPPLYDLATPVIKFIDFAVSQNVKKFVLLSASVIQKGGPAMGKIHGHLDSIKDVTYTVLRPTWFMENFSTKGEIQCEAIRRDSTVYSATENGKIPFISVVDIARVAACALTAETLKNSDHILQGPDLLTYDEVAQALTGVLGRKITHTKMTEGELAEKLMEEGVTPEEAYMHAAMDSMIKSGSEERVVSDEVKEWTGVKPRGFINFALSEKAAWRARK

SEQ ID NO: 209 - protein, 291 aa, synthetic construct
MSSTILLTGGNGKTARHIASLLEEANLPFVIGSRSSRPQNIGRHRSFDWLDEKTFQNILTPGEGMEPISAVWLVPPPISDLASPVNKFIDFACSKDVTRFVLLSASIVEKGGPAMGQIHAHLDSLEGISYTVLRPTWFMENFSTKGDFQCDTIRKEDTIYSATGDGKIPFISVVDIARVALRALTANVPDQMDHVLLGPELLTYDDVAETLTRILDRKITHARLTESELVQKMQKSGMSAEDAQMHASMDLIVQSGAEDRLNTVVRELTGSEPRNFSDFALNEAKLWVSRN

SEQ ID NO: 210 - protein, 290 aa, synthetic construct
MSSTTLLLGGHGKTALQIAAILRASNQSFLVASRTASAGSTYTHAKFDWFDERTYESPFLKASSEGMKPIASVWLVAPPIFELAPPMIKFVEFARAREVKRFVLLSASTIEKGGPAMGQVHAHLDTIKGIVYAVLRPTWFMDNFSTAGELQQLAIKNDSKIYSAAGDGKIPFISAKDIAKVAVKCLTNGNLQAKEYVLLGPKLHTYDEVASIISGVVGREIVHVKLSEADFTKHLISRGVPLSDAEFMAIMETGIKNGGQESLNDVVEEVTGSPPETFTDFAFRHKECWM

SEQ ID NO: 211 - protein, 291 aa, synthetic construct
MSSTILLTGGRGKTASHIAALLDAANLPFAIASRSCSHDSKYRQLRFDWLDDTTYDNVLSPKDGMQPISAVWLVPPPIADLAPPVNAFINHARLKDVKRFVLLSASMLERGSPAMGQIHEHLASMEGIHYTVLRPTWFMENFSSPHEFQCKAIRKEDKIYSAAENGKVPFISVVDIARVAFRALKGDVEENIDPILLGPELLTYDDVAETLSKQLNRKIDHVRLEESELAEKMHEYGMPLEDAQMHASMDLIVRAGGEERLNKTVLELTGTTPRTFQEFASSEKDIWLLKN

SEQ ID NO: 212 - protein, 292 aa, synthetic construct
MSSTILVLGGRGKTASRLSLLLDNAGVPFLVGSSSTSYVGPYKMTHFDWLNEDTWTNVFLRASLDGIDPISAVYLVGGHAPELVDPGIRFINVARAQGVNRFVLLSASNIAKGTHSMGILHAHLDSLEDVQYVVLRPTWFMENLLEDPHVSWIKKEDKIYSATGDGKIPFISADDIARVAFSVLTEWKSQRAQEYFVLGPELLSYDQVADILTTVLGRKITHVSLAEADLARLLRDDVGLPPDFAAMLASMETDVKHGTEVRNSHDVKKVTGSLPCSFLDFAEQEKARWMRH

SEQ ID NO: 213 - protein, 291 aa, synthetic construct
MSSTILLTGGNGKTARHIAGLLEEANLPFIIGSRSNSPQTIERHRSFDWLDETTFHNILSPGEGMRPISAVWLVPSPIPDLASPVNKFIDFACSKDVKRFVLLSASIVEKGGPAMGQIHAHLDSLEGISYTVLRPTWFMENFSTMDDFQYEAIRKEGKVYSATGDGKVPFISVVDIARVALRALTGDVPDKTDHILLGPELLTYDDVAETLTRTLDRKITHARLTESELAEKMQKSGMPAEDAQMHASMDSIIQSGAEDRLNTAVRDLTGLEPRNFSDFALSEKKFWRSRD

SEQ ID NO: 214 - protein, 287 aa, synthetic construct
MAAAILLTGGTGKTSIRLARFLQNANIPFLLASRRGSSAAPVGMPAVKFDWLDKETWKAPFGGKPISAIYLMEPLVAEPWKPMIEFIEYTRNVHGVTRFVLLAGTSTDLSRPGMGVVWKHFLETGVDHCVLRPSWFMENLTDEAPGPLIREYSKIYTACGDGKIPFVSAIDIAAVAFRALTDPKSHNCDYRILGPELLTYDEVAEKLSAHLGRRIEHVKLSGDERYKGLTDAGVSDYFARFLTNLEVAAADGFETHMNNTVEEVRAGHPGAWTTLLMKIYIRGSRYN

SEQ ID NO: 215 - protein, 494 aa, synthetic construct
MSSGSTLIAISVTLFLPLIISAFYRLYKHPLRKIPGPRLAAVTNLYAFYYNYIQEGGYSKQFKGLHEKYNSPVVRIGPNHVHVDKLSFYDEVFRPGTKYRKDPSFYNSFGGLDGMIDEDGFRTYRSHISSLYSAKSVDMLAPKLLREVQTVADRLQKDVGTNQVVNIQKMFRTLSADMVLHILFPQDIDLFECDQYHPFLESFDIMLTKTWLSLSYPFALGLLALLPWPEYKRVGVAAENFAKYCQGWMTKAQHLQNSGAEKGRDCHIKRYLELDPTNEKKKKAIPYPLEDIFNFIAGGTDTTAYIISCVVYYLLKSPNVLSNLQKELDEAALCIRHKFDHKQIQSLPYLTAVIKEALRISTPAPGCLPRIVPDGGAHVDSVYIPSGTAVSVSMLSIQQNADLFPEPRKFKPERWLCEEGKALAKWNIAFSRGPRQCIGTNVAYLELYGTIAYLFSRFEFALSGNDGDELEWIDRFTAHNIEDIKVKITKDKWT

SEQ ID NO: 216 - protein, 520 aa, synthetic construct
MSSATNPSTLLSVTKQEQGLSLRWPTQYGSWTWILLTLCISLTTPFIVNCVYNLYLHPLRNVPGPKLAAVTSFYAFYWNWIRDGEYSKKFNFWHKHYNSPVIRIGPNHVHTTEADIYYVIFKPGSKWLKDKAFYKHFNGVDAMIEPTLYRTYRMHLAPLYSQRTIDDLAPKVYQNLSIFGNRMTKMAETGKAVNMVKIMRTLSTSMILHIVYSLDIPLCEHDGYHPLLEAFERVMTQSWLFVTYPIIFGLLSLIPGTTIARLNSSFEVFMKYCKEWNDEDMRKQRTSEGQSARDSHMKRYLEIQDDEEKNHIIPNPIDDVFNFIAGGSDTTTYTTACAFFYILSSPMVHKKILDELDANSGAIRDAYDYNKIKNLPYLNAVIKETLRISVPLPGSMPRVVPEGGVVLGSFSIPAGTAVSISHQAISFNEEIFPSSGEFIPERWIEAAGLDKWNIAFSRGPRQCPGKTLAFLELHCVLAYFFSRFEMTLTGNCGKSLRWVDRYVAANLDDVEVRILRDRWN

SEQ ID NO: 217 - protein, 509 aa, synthetic construct
MAASLQWLQQTRHELSWTWILLTTCIALTSPLVLKGIYNVYFHPLRNIPGPKLAALTDFYAFYWNWIRDEGYSKQFSRLHEQYNSPIIRIGPNNVHTTQVEFYDVIFKSGSKWLKDKSFYKYFNGLDAMIEPYQYRTYRTHLAPLYAQRAIDGLAPKLRSDLTNSASGMMRQTENGQTVNMAKVLRTLSTSMILHNLFSLDISLNDGDEYHPFLEAFEQLMTQSWLFVTYPMVPMVLSLIPGTSFARFNSSYTTFSNYCTAWNDEDMRKQRESEGQSTRDSHTRRYLSLKDDDARKKTAIPYPLDDVFNFVAGGSDTTAYTTACAFYHILSSPTVRENLVVELDEHSSIIRDEFDYNKIQNLPYLNAVIKETLRISVPVPGSLPRIVPQGGITIGSFSLPAGTGVSITQQAISFNEKIFPLPHSFLPERWIGPKSVGLDKWNIAFSRGPRQCIGTTLAYLELRCVIAYFFSRFDMALTGNCGDQLRWVDRFVAVNLDDVEVQILADRWT

SEQ ID NO: 218 - protein, 508 aa, synthetic construct
MAAILPWLSQLQSVSLGTIFLTLFLVILTPLVFTSVYRLYFHPLRKIPGPRTGALTSFYGFYWNWIRDEGYSKLFNPLHKQYNSHIIRIGPNHVHINQPQAFDEIFKVGTTWRKDSSFYKYFNGLDAMIEPTQYRTYRTHLAPLYAQRSIDGLTPKLHDDLVVTAERMAKSIENGEPVNMVKILRTLSTSMMLYTLYSQDIPLSQYDGYHPFLEAFELLMTQSWLMINYPMMGMILGLIPGTSFAKFNAAFGTFLKYCKEWNDEDERIQKLETAESLRDSHMKRYLAIDPNNEIKKKVVPHPLEDIFNFIAGGSDTTSYTAACAFFHVLSSSEVHSKLVAELDQASSVIRDTFDYNKIQNLPYLNAVIKETLRISCPVPGCLPRVVPEGGMNLGSVNLPAGTVVSISQLAIHFNETIFSSPDKFIPERWLGDDRKSIEKWNIAFSRGPRQCIGTTLAYMELRCVLAYFFSRFEFKLTGSCGDKLRWVDRFVSVNLDDVEVTIVKDRWA

SEQ ID NO: 219 - protein, 508 aa, synthetic construct
MAASLQWLYQAQCPSWTWIMFTACISITFPLVMIGIYNIYFHPLRNVPGPKLAALTNFYAFYWNWIRDEGYSRQFQSLHTYYNSSVLRIGPNHVHTTEIELYNVIFKPGSKWLKYKSFYRYFNGLDAMIEPSQYRTYRAHLAPLYAQRAIDDLSPKIHDDLSSSAARMIKITETGETINMVKVLRTLSTSMILYNLYSIDISLNECDGYHPFLEAFEQLMAQSWLFVTYPMVPMLLGLIPGTSFARLNSCYTKFMNYCIAWNHEDVRKQQASEEQSTRDSHTKRYLAIKNDDGLKKSIVPNPLDDVFNFIAGGSDTTSYTAACAFFHILSSPIICAKLVTELDEHSSIIRDAFDYNKIQTLPYLNAVIKETLRISVPVPGCLPRVVPEGGITLGSLSLPAGTAVSISQQAISLNNEIFSSPHRFSPERWIGPEAVGLDKWNIAFSRGPRQCIGTTLAYLELRCILAYFFSRFEMTLTGNCGDHLRWVDRFVAVNLDDVEVLILRDRWS

SEQ ID NO: 220 - protein, 514 aa, synthetic construct
MAAENNFTTASTVSISESGLIWQRLSQLQQASWALIIFTTCVAVLLPAVITGVYRLYFHPLRNVPGPKLAALTNFYAFYWNWIRDEGYSRRFSALHKYYNSPIIRIGPNSVHTNQPDDFDVIFKVGSKWKKDNSFYKYFNGLDAMIDPAQYRTYRSHLAPLYAQRAVNDLAPKLHKDLLSSAAKMEKSMKTSEAVNMVKVLRTLSTSMILHNLYSHDISLIEYDGYHPFLEAFEQLMTQSWLWFIPGTSFAKFNSSYTTFMKYCKGWNDEDMRIQRLTENQELRDSHMKRYLAMNSNDWAKDNVVPNPLNDIFNFVAGGSDTTSYTLACAFFYILSSPEIYAKLVKELDENVAFIRDTFDYNKIQNLSYLNAVIKETLRISVPVPGCLPRIVPESGTTLGSVYLPAGTAVSISQQSISFHETIFPSPEKFIPERWLGNEAQRLDKWNVAFSRGPRQCIGTSLAYLELRCVLAYFLSRFEMALTGNCGDRLRWVDRFVAVNLDDVEVKLLRDRWN

SEQ ID NO: 221 - protein, 499 aa, synthetic construct
MAAGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLRIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVSPSPYELCAVPR

SEQ ID NO: 222 - protein, 502 aa, synthetic construct
MAAGLLIGDVLGPLAIAVALFLLLVDLLHRRRFWAARFPPGPMPLPLLGNLLQMDFQDTLFYFDRLRRRFGDVFSLQLAWTPVVVLNGLEAVREALVYHSEDTADRPPMPIYEHLGFGPRSQGVFMARYGREWREQRRFALSTLRNFGLGKKSLEQWVTEEASCLCAAFADHAGRPFSPKALLNKAVSNVITSLTYGRRFGYDDPRLHKLIDGALKGLQEDSGFAREALNSIPVLLRIPGLADKVFSSQKALLTLLNELVQEHRITRDPAQPPQDLTDAFLDEIEKARGNPESSFNDENMLMVTADLFLAGMLSTSTTLAWALLLMILHPDVQRRVQQEIDEVLGPVQRPAMADQTRMPFTMAVMHEVQRFGDLVPLGMPHMTSRDIEVQGFLIPKGTTLITNLSSVLKDKTVWKKPFRFHPEHFLDAQGQFVKQEAFMPFSAGRRVCLGEPLARMEFFLFFTCLLQRFSFSVPVGQPRPSDHGVFSFLMIPPPYQLCAVPR

SEQ ID NO: 223 - protein, 495 aa, synthetic construct
MAASALGVTVALLVWAAFLLLVSMWRQVHSSWNLPPGPFPLPIIGNLFQLELKNIPKSFTRLAQRFGPVFTLYVGSQRMVVMHGYKAVKEALLDYKDEFSGRGDLPAFHAHRDRGIIFNNGPTWKDIRRFSLTTLRNYGMGKQGNESRIQREAHFLLEALRKTQGQPFDPTFLIGCAPCNVIADILFRKHFDYNDEKFLRLMYLFNENFHLLSTPWLQLYNNFPSFLHYLPGSHRKVIKNVAEVKEYVSERVKEHHQSLDPNCPRDLTDCLLVEMEKEKHSAERLYTMDGITVTVADLFFAGTETTSTTLRYGLLILMKYPEIEEKLHEEIDRVIGPSRIPAIKDRQEMPYMDAVVHEIQRFITLVPSNLPHEATRDTIFRGYLIPKGTVVVPTLDSVLYDNQEFPDPEKFKPEHFLNENGKFKYSDYFKPFSTGKRVCAGEGLARMELFLLLCAILQHFNLKPLVDPKDIDLSPIHIGFGCIPPRYKLCVIPRS

SEQ ID NO: 224 - protein, 495 aa, synthetic construct
MAAAVLGITVALLVWIATLLLVSIWKQIYRSWNLPPGPFPIPFFGNIFQLDLKDIPKSLTKLAKRFGPVFTLHLGQRRIVVLHGYKAVKEVLLNHKNEFSGRGDIPVFQEYKNKGIIFNNGPTWKDVRRFSLSILRDWGMGKQGNEARIQREAHFLVEELKKTKGQPFDPTFLIGCAPCNVIADILFNKRFDYDDKKCLELMSLFNENFYLLSTPWIQAYNYFSDYLQYLPGSHRKVMKNVSEIRQYTLGKAKEHLKSLDINCPRDVTDCLLIEMEKEKHSQEPMYTMENISVTLADLFFAGTETTSTTLRYGLLILMKYPEIEEKLHEEIDRVIGPSRAPAVRDRMNMPYMDAVVHEIQRFINLVPSNLPHEATRDTVFRGYVIPKGTVVIPTLDSLLFDNYEFPDPETFKPEHFLNENGKFKYSDYFKAFSAGKRVCVGEGLARMELFLLLSAILQHFNLKSLVDPKDIDLSPVTIGFGSIPREFKLCVIPRS

SEQ ID NO: 225 - protein, 505 aa, synthetic construct
MAAALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 226 - protein, 679 aa, synthetic construct
MAAGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 227 - protein, 697 aa, synthetic construct
MAAAQLDTLDIVVLVVLLVGSVAYFTKGSYWAVPKDPYAAANSAMNGAAKTGKTRDIIQKMEETGKNCVIFYGSQTGTAEDYASRLAKEGSQRFGLKTMVADLEDYDYENLDKFPEDKIAFFVLATYGEGEPTDNAVEFYQFITGEDVAFESGASAEEKPLSSLKYVAFGLGNNTYEHYNAMVRHVDAALTKLGAQRIGTAGEGDDGAGTMEEDFLAWKEPMWAALSESMNLQEREAVYEPVFSVIEDESLSPEDDSVYLGEPTQGHLSGSPKGPYSAHNPYIAPIVESRELFTAKDRNCLHMEIGIAGSNLTYQTGDHIAIWPTNAGVEVDRFLEVFGIEKKRHTVINIKGLDVTAKVPIPTPTTYDAAVRFYMEICAPVSRQFVSSLVPFAPDEESKAEIVRLGNDKDYFHEKISNQCFNIAQALQNITSKPFSAVPFSLLIEGLNRLQPRYYSISSSSLVQKDKISITAVVESVRLPGASHIVKGVTTNYLLALKQKQNGDPSPDPHGLTYAITGPRNKYDGIHVPVHVRHSNFKLPSDPSKPIIMVGPGTGVAPFRGFIQERAALAESGKDVGPTILFFGCRNRNEDFLYKEEWKVYQEKLGDKLKIITAFSRETAKKVYVQHRLQEHADLVSDLLKQKATFYVCGDAANMAREVNLVLGQIIAKSRGLPAEKGEEMVKHMRSSGSYQEDVWS

SEQ ID NO: 228 - protein, 697 aa, synthetic construct
MAAAQLDTLDLVVLAVLFVGSVAYFTKGTYWAVAKDPYASTGPALNGAAKAGKSRNIVEKLEETGKNCVIFYGSQTGTAEDYASRLAKEGSQRFGLKTMVADLEDYDYENLDQFPDDKVAFFVLATYGEGEPTDNAVEFYQFLTGEDIAFESGASAEDKPLSSLKYVAFGLGNNTYEHYNSMVRNVDAAFQKLGAQRIGTAGEGDDGAGTMEEDFLAWKEPMWAALSEAIGLEEREAEYEPVFSVTEDDSLSPEDDSVYLGEATKGHLEGSPKGPFSAHNPYIAPIVESRELFTVKDRNCLHMEISIAGSNLSYQTGDHIAIWPTNAGAEVDRFLQVFGLEGKRHTVINIKGIDVTAKVPIPTPTTYDAAVRYYLEVCAPVSRQFVSSLAAFAPDEESKAEIVRLGSDKDYFHEKISNQCFNIAQALQSITSKPFSAVPFSLLIEGLTKLQPRYYSISSSSLAQKDKISVTAVVESVRLPGASHIVKGVTTNYLLALKQKQNGDPSPDPHGLTYEINGPRNKYDGIHVPVHVRHSNFKLPSDPSRPVIMVGPGTGVAPFRAFIQERAALAARGEKVGPTVLFFGCRNRNEDFMYQDEWKIYQEQLGDSLKIITAFSRESSQKVYVQHRLSEHAELVSDLLKQKATFYVCGDAANMAREVNLVLGQIIAKQRGLPAEKGEEMVKHMRSSGSYQEDVWS

SEQ ID NO: 229 - protein, 694 aa, synthetic construct
MAAAELDTLDIVVLGAILFGTLAYFTKGTLWGVTKDPYADAFAKVSGAKAGRSRNIIEKMEESGKNCVIFYGSQTGTAEDYASRLAKEGKSRFGLETMVADLEEFDYDNLDTMPSDKVAMFILATYGEGEPTDNAVEFYEFISGEDVSFSEGNDPPLDNLNYVAFGLGNNTYEHYNSMVRNVDKALQKLGAHRIGEAGEGDDGAGTMEEDFLAWKDPMWAALAEKMGLEEREAVYEPTFAIVHRENLTPESPEVYLGEPNKMHLDGIAKGPFNSHNPYIAPITESRELFSTKDRNCIHMEVDISGSNLSYQTGDHIAVWPTNPGDEVDRFLDIVGLKEKRNHVISVKALEPTAKVPFPTPTTYDAIVRYHLEICAPVSRQFVATLAAFSPDEDTKAEMTRLGSDKDYFHDKTAPHYYNIARFLEAVGKGQKWTNIPFSAFIEGLTKLQPRYYSISSSSLVQPKKIAITAVIESQVIPGRKDPFRGVATNYLFALKQKQNGDPNPTPFGQTYALNGPRNKFDGIHVPVHVRHSNFKLPSDPARPIIMVGPGTGVAPFRGFVQERAKQAQDGVAVGKTILFFGCRKRTDDFLYESEFEEYKKILGDSFEIVTAFSRETPEKVYVQHRLKGRSKEVGELLSQKAYFYVCGDAAHMAREVNTVLAQIIAEARGVPEAKGEEIVKNMRAANQYQEDVWS

SEQ ID NO: 230 - protein, 694 aa, synthetic construct
MAAAELDTLDMVVLGVILLGTLAYFSKGTIWGVVKDPYANTFANVNGAKAERSRNIIEKMEESGKNCVIFYGSQTGTAEDYASRLAKEGKSRFGLETMVADLEEYDYDNLDTMPSDKVAMFVLATYGEGEPTDNAVEFYEFITGEDVSFSEGGDDPLQNLNYVAFGLGNNTYEHYNSMVRNVDKALTKLGAHRIGQAGEGDDGAGTMEEDFLAWKDPMWVALAEKMGLEEREAVYEPTFSIVDRENLTPESPEVYLGEPNKMHLDGTAKGPFNSHNPYIAPIVESRELFSAKDRNCIHMEIDVSGSNLTYQTGDHLAIWPTNPGEEVDRFLDVIGLKEKRNNVISVKPLEPTAKVPFPTPTTYDAIVRYHLEICGPVSRQFAATVAAFSPDEEIKAEMTRLGSDKQYFHEKTGPHFYNIARFLEAIGKGQKWSNIPFSAFIEGLTKLQPRYYSISSSSLVQPKKISLTAVVESQKIVGRMEPFRGVATNYLFALKQKQNGDPDPSSFGKTYALTGPRNKFDGIHVPVHVRHSNFKLPSDPGKPIIMVGPGTGVAPFRGFIQERAKQAQNGTSVGKTILFFGCRKRSEDFLYESEFEEYKKTLGDNLEIVTAFSREGSEKVYVQHRLKERSKEVGELLSQRAYFYVCGDAAHMAREVNTVLAQILAESRGVSEAKGEEIVKNMRAANQYQEDVWS

SEQ ID NO: 231 - protein, 698 aa, synthetic construct
MAAAQLDTLDIIVLAALLVGTVAYFTKGTYWAVYADPYGNSLGSANGAAKAGKSRNILEKMEETDKNCVVFYGSQTGTAEDYASRLAKEGHARFGLNTMVADLEEYDFDNLDSFPEDKLAVFVLATYGEGEPTDNAVEFYEFIGSEDVSFADGGSADDKPLSKLHYVAFGLGNNTYEHYNSMVRNVDKYLTKLGASRLGTAGEGDDGAGTMEEDFLAWKEPMWAAVSEKMGLEEREAMYEPTLEINERPELSVEDDTVYLGEPNKNHLEGNQKGPFNANNPFIAPIVESAELFTSNDRNCLHMEISIAGSNLSYTTGDHIAIWPNNAGKEVDRVFKVLGMEDRRHTVIAVRGLDPTAKVPFPSPTTYDAAVRYHIEINAAVSRQLVSVIAQFAPNEEIKAEMVKLGSDKDYFREHVTERNLNLAQLLEINGKGQTWDKIPFSFLFETMVKLQPRYYSISSSSLVQKDKIAITAIVESIEKPGAPYALKGVTTNYLLALKQKQHGAPEPDPHGLNYAITGPRNKYDGIHVPVHVRHSNFKLPSDPSKPIIMVGPGTGVAPFRGFVQERAAQAQAGQSVGKTVLFFGCRKRSEDFMYADEWKQYQEALGDNFKMHTAFSRDGPKKVYVQHKLEEFGEEVNKLLEQKAYFYVCGDAAHMAREVNTLLGKIISQYRNVSETKGEEIVKAMRASNQYQEDVWS

SEQ ID NO: 232 - protein, 694 aa, synthetic construct
MAAAELDTLDIIVLGVILLGTLAYFTKGTLWGVTKDPYANAFANVSGAKAGRSRNIIEKMEESGKNCVIFYGSQTGTAEDYASRLAKEGKSRFGLETMVADLEEYDYDNLDTMPSDKVAMFVLATYGEGEPTDNAVEFYEFITGEDVSFSEGSDPPLENLNFVAFGLGNNTYEHYNSMVRNVDKALQKLGAHRIGEAGEGDDGAGTMEEDFLAWKDPMWAALADKMGLEEREAVYEPTFDIVNRENLTPESPEVYLGEPNKMHLEGAAKGPFNSHNPYIAPIAESRELFSAKDRNCIHMEVDISGSNLSYQTGDHIAIWPTNSGDEVDRFLDIIGLKEKRNYVISVKALEPTAKVPFPTPTTYDAIVRYHLEICAPVSRQFAATLAAFSPDEEVKAEMTRLGSDKQYFHDKIGPHFYNIARFLEAVGKGQKWTNIPFSAFIEGLTKLQPRYYSISSSSLVQPKKISITAVVESRKIPGRNDPFNGVATNYLLALKQKQNGDPNPSPFGKTYALNGPRNKFDGIHVPVHVRHSNFKLPSDPGKPVIMVGPGTGVAPFRGFIQERAKQAQNGATVGKTILFFGCRKRSEDFLYETEFEEYKKALGDSLEIVTAFSRESSKKVYVQHRLKERSKEVGELLSQKACFYVCGDAAHMAREVNTVLAQILAESRGVSEAKGEEIVKNMRAANQYQEDVWS

SEQ ID NO: 233 - protein, 773 aa, synthetic construct
MAAAQLDTLDIIVLAAICLGTVAYFTKGTYWGITQDPYASTYSAANSSKAGKTRNILEKMEESNKNCVVFYGSQTGTAEDYASRLAKEGKSRYGLETMVADLEDYDYENLDSFPEDKIAMFVLATYGEGEPTDNAVDFYEFITGESPSFSNESEPPLGNLKFVAFGLGNNTYEHYNSMVRDVTKALEKLGATKIGAAGEGDDGAGTMEEDFLAWKDPMWVDLAEKMGLEEREAVYEPVFAITEREGLTKDSPEVYLGEPNKMHLEGASKGPYNAHNPYIAPIAESREIFTVKDRNCLHMEIDISGSNLSYQTGDHVAVWPTNAGREVDRFLKVTGLLPKRDTVVGVKALDPTAKVPFPTPTTYDAIVRYHMEIGAPVSRQFLATLAPFAPTEAAKAEMAKLGSDKDYFHEKISNQYLNIAQVLQAVAGDEKWTSIPFSAFIEGVNKVQPRYYSISSSSLVQKKKISVTAIVESMHIPGREDALKGVTTNYLLALKQKQNGDPSPDPHGLTYDITGPRNKYDGVHVPVHVRHSNFKLPSDPSKPIIMVGPGTGVAPFRAFVQERAQQAKNGENVGRTLLFFGCRKSTEDFLYSDEWETYKKDLGDKFELITAFSRETPGKKVYVQHRLSERAKEVNELLQQKAYFYVCGDAANMAREVNTILGKIIAEQRGIPETKAEDIVKGMRSANQYQVSSLNSTSFEPTSTLLTAPGSSVRDFDCDIMFPMYHRSWIGPCTIPAYTSQTDADFNFLGGCVVMNTKSLLLRYDDSTFIFWG

SEQ ID NO: 234 - protein, 694 aa, synthetic construct
MAAAEFDTLDIVVLGVILLGTIAYFTKGTLWGITKDPYANAFANANGVKAGRSRNILEKMDETGKNCVIFYGSQTGTAEDYASRLAKEGKSRFGLETMVADLEEYDFDNLDAMPSDKIVMFILATYGEGEPTDNAVEFYEFITGEDVSFSEGSEPALQNLNYVAFGLGNNTYEHYNSMVRNVDKALQKLGANRIGEAGEGDDGAGTMEEDFLAWKDPMWAALAEKMGLEEREAVYEPTFGIVDRENLTVDSPEVYLGEPNKMHLEGTAKGPFNSHNPYIAPIAESRELFSAKDRNCIHMDVDISGSNLSYQTGDHIAIWPTNSGDEVDRFLDIVGLKEKRNNVISVKALEPTAKVPFPTPTTYDAIVRYHLEICAPVSRQFVATLAAFAPNDEAKAEMARLGSDKDYFHSKTGPHFYNIARLLDTVGKGEKWTNIPFSAFIEGLNKLQPRYYSISSSSLVQPKKISITAVIESQAIPGRQDPFRGVATNYLFALKQKQNGDPNPSPFGKTYALNGPRNKFDGIHVPVHVRHSNFKLPSDPAKPVIMVGPGTGVAPFRGFIQERARQAQNGATVGRTILFFGCRKRSEDFLYESEWEEYKKALGDSLEIVTAFSRESSKKVYVQHRLKERSKEIGELLSQKAYFYVCGDAAHMAREVNTVLAQIIAESRGVSETKGEEIVKNMRAANQYQEDVWS

SEQ ID NO: 235 - protein, 697 aa, synthetic construct
MAAAQLDTLDLVVLVALLVGSVAYFTKGSYWAVPKDPYASSASTLNGAAKSGKTRDIVEKMDETNKNCVIFYGSQTGTAEDYASRLAKEGSQRFGLKTMVADIEDYDYENLDKFPDDKVAFFVLATYGEGEPTDNAVEFYQFFTGEDVAFESGASADDKPLSSLKYVTFGLGNNTYEHYNAMVRQVDTALTKLGAQRIGTAGEGDDGAGTMEEDFLAWKEPMWAALSDAMNLQEREAVYEAVFSVVEDDEKSPEEESVYLGEPTASHREGRAKGPFSAHNPYVAPIVESSELFTVKDRNCLHMEISIAGSNLSYQTGDHIAIWPTNAGAEVDRFLQVFGIEEKRHSVINIKGIDVTAKVPIPTPTTYDAAVRYYMEVCAPVSRQFVSTLAAFAPDEDSKTEIVRLGNDKDYFHEKITNQCFTIAQALQSITSKPFTAVPFSLLIEGLNKLQPRYYSISSSSLVQKDKISITAVVESVRLPGASHIVKGVTTNYLLALKQKQNGDPSPDPHGLTYSINGPRNKYDGIHVPVHVRHSNFKLPSDPSKPIIMVGPGTGVAPFRGFIQERAALAAKGEKVGTTLLFFGCRKSTEDFIYKDEFKTFQDQMGDSLKIITAFSREGSEKVYVQHRLKENAELVSELLKQKANFYVCGDAANMAREVNLVLGHIIAQQRGLPAEKGEEMVKHMRSSGSYQEDVWS

SEQ ID NO: 236 - protein, 697 aa, synthetic construct
MAAAQLDTLDLVVLVALLVGSVAYFTKGSYWAVPKDPYASSAATQNGAAKSGKTRDIVEKMEETNKNCVIFYGSQTGTAEDYASRLAKEGSQRFGLKTMVADIEDYDYENLDKFPDDKVAFFVLATYGEGEPTDNAVEFYQFFTGEDVAFESGASAEDKPLSSLKYVTFGLGNNTYEHYNAMVRQVDAALTKLGAQRIGTAGEGDDGAGTMEEDFLAWKEPMWAALSEAMSLQEREAVYEAVFSVVEDDEKSPEDESVYLGEPTASHREDRAKGPFSAQNPYIAPIVESSELFTVKDRNCLHMEISIAGSNLSYQTGDHIAIWPTNAGAEVDRFLQVFGLEEKRHSVINVKGIDVTAKVPIPTPTTYDAAVRYYMEVCAPVSRQFVSSLAAFAPDEESKVEMVRLGNDKDYFHEKIADQRFTIAQALQSITSKPFTAVPFSLLIEGLNKLQPRYYSISSSSLVQKDKISITAVVESVRLPGASHMVKGVTTNYLLALKQKQNGDPSPDPHGLTYSITGPRNKYDGIHVPVHVRHSNFKLPSDPAKPIIMVGPGTGVAPFRGFIQERAALVAKGEKVGTTLLFFGCRKSDEDFMYKDEFKTFQDQMGDSLKIITAFSREGAEKIYVQHRLKENAELVSDLLKQKANFYVCGDAANMAREVNLVLGHIIAQQRGLPAEKGEEMVKHMRSSGSYQEDVWS

SEQ ID NO: 237 - protein, 694 aa, synthetic construct
MAAAELDTLDLVVLAAILLGTLAYFTKGKYWGVVKDPYATAFANANGVKAGRTRNIVEKMDESGKNCVIFYGSQTGTAEDYASRLAKEGKSRFGLETMVADLEEYDFDNLDTVPTDKVVMFVLATYGEGEPTDNAVDFYEFITADDVALSEGSDPALGNLNYVAFGLGNNTYEHYNSMVRNVDKALQKLGAHRIGEAGEGDDGAGTMEEDFLAWKDPMWAALASKMGLEEREAVYEPTFGIVDRETLTRDSVEVYLGEPNKMHLEGTSKGPFNAHNPYIAPIAESRELFSAKDRNCIHLDIDVSASNLNYQTGDHIAVWPTNPGDEVDRFLDVIGLTEKRNDVISVKALEPTAKVPFPTPTTYDAIVRYHLEVCAPVSRQFVATLAAFSPSDDIKAEMTRLGNDKDYFHDKTGPHFYNIARLLATVGKGQKWTNIPFSAFIEGLNKLQPRYYSISSSSLVQPKKISITAVVESQMIPGRNEPFRGVATNYLFALKQKQNGDPNPQPFGQSYELNGPRNKYDGVHVPVHVRHSNFKLPSDPGKPVIMIGPGTGVAPFRGFVQERAKQAREGANVGRTLLFFGCRKSTEDFMYESEWAEYKKDLGDKFEMVTAFSRETNKKVYVQHRLKERAKEVNELLQQKAFFYVCGDAANMAREVNAVLAQIIAEGRGVSEQKGEEVVKHMRAANQYQEDVWS

SEQ ID NO: 238 - protein, 696 aa, synthetic construct
MAAAQVDTLDMLVLAVILVGTIAYFTKGKYWAVVKDPYASSYAATNSAKQGQTRNIVEKLEETGKNCVIFYGSQTGTAEDYASRLAKEGSQRFGLKTMVADLEDYDYACLDKFPEDKVAFFVLATYGEGEPTDNAVEFFQFITGDDASFEDGGSPEDQPLSKMRYVAFGLGNNTYEHYNAMVRIVDKALTKCGAKRIGDAGEGDDGAGTMEEDYLAWKEPMWKALAEEMGLEEREAIYEPTFSVIEEADLNTESENVYLGEPNSSHLKGRGQGPYNAHNPFLSPIVESRELFSVKDRNCLHMEFDIKGSQLNYQTGDHIAIWPTNAGQEVDRFLKVFGLEEKRHTVVRVKPIDVTAKVPFPQPTTYDSVVRYYIETCGPVSRQFISQLAAFAPDEETKKKMIRIGEDKDVFSDKVSAFYFNIAQALQSFTDKPFTAVPFSLLIEGIPKIQPRYYSISSSSIVQKEKISITAVVESLRVPGAGHVVKGVTTNYLLALKQKQHGEPNPDPFGLTYAISGPRNKYDGFHVPVHVRHSNFKLPSDPSKPIIMVGPGTGVAPFRGFIQERVHQAEKGETVGPTILFYGCRKSTEDFLYKEEFEEVSKKLGDSFKLITAFSRETSQKVYVQHRLKEHAELVSDLLSKKANFYVCGDAANMAREVNVVLGQILAEQRGLKPEKGEELVKHMRNTGSYQEDVWS

SEQ ID NO: 239 - protein, 391 aa, synthetic construct
MAARILKSHPLLKIVNSYIIDSPQPANISYLWNFGSLLALCLGIQIVTGVTLAMHYTPSVLEAFNSVEHIMRDVNNGWLVRYLHANTASAFFFLVYLHIGRGLYYGSYKAPRTLTWAIGTVILIVMMATAFLGYVLPYGQMSLWGATVITNLMSAIPWIGQDIVEFIWGGFSVNNATLNRFFALHFLLPFVLAALVLMHLIAMHDTVGSGNPLGISGNYDRLPFAPYFIFKDLVTIFIFFIVLSIFVFFMPNALGDSENYVMANPMQTPPAIVPEWYLLPFYAILRSIPNKLLGVIAMFSAILALMVMPITDLSKLRGVQFRPLSKVAFYIFVANFLVLMQLGAKHVETPFIELGQISTVLYFAHFFVIVPVVSLIENSLVELSTKYNSSC

SEQ ID NO: 240 - protein, 411 aa, synthetic construct
MAARILKSHPLLKLANAYLVDHSQPSNISYWWNFGSLLGLCLLIQILTGVFLAMHYNASIAEAFNSTEHIMRDVQNGWIIRYLHANTASAFFFCVYMHLSRGLFYGSYRSPRTLAWVIGTIILILMMATAFLGYLYVLPFGQMSATVITNLLSAIPWIGQDVVEFLWGGFSVNNATLNRFFALHFILPFVLAALAILHLIAVHETAGASNPLGVPGYYDRVPFAPYFIYKDLITIFLFFFILSVFVFFLPNQLGDCENYVVANPMQTPAAIVPELLPFYAILRSIPNKLLGVIAMFAALVSILVLPYSDIGRTKGLQFRPLSKLIFFMFVANFLLLLQLGAKHVESPFIELGQISTAFYFAYFFLLVPSVSILENTLLDIKTYALNNKKKIVLYTVYTLIANRMFKVRLLA

SEQ ID NO: 241 - protein, 389 aa, synthetic construct
MAARILKSHPFLKLLNAYLIDHSQPSNISYLWNFGSLLGLCLGIQIITGITLAMHYNPSVAEAFNSVEHIMRDVNNGWLVRYLHSNTASAFFFLVYLHIGRGLYYGSYRAPRTLAWVLGAIILILMMGTGFLGYVLPYGQMSLWGATVITNLISAIPWIGQDIVESHKCSVNNATLNRFFALHFVLPFVLAALALMHLIAVHESAGASNPLGVPGYYDRVPFAPYFLFKDLITIFIFFFVLSMFVFFMPNVLGDSENYVVANPMQTPAAIVPEWYLLPFYAILRSIPNKLLGVIAMFAALVAILALPYTDLGRTKGLQFRPLSKILFYIFVANFLILQQLGAKHVETPFIEFGQISTALYFAHFFILVPVVSVIENTLIDLYQINNKSR

SEQ ID NO: 242 - protein, 390 aa, synthetic construct
MAARIFKSHPLLKLINSYIIDSPQPANLSYLWNFGSLLAFCLAIQIVTGVTLAMHYNPSVLEAFNSVEHIMRDVNNGWLIRYLHSNTASAFFFLVYLHIGRGLYYGSYRAPRTLVWTIGTVIFILMIVTAFLGYVLPYGQMSLWGATVITNLMSAIPWIGQDIVEFLWGGFSVNNATLNRFFALHFVLPFVLAALVLMHLIALHDSAGSGNPLGVSGNYDRLPFAPYFLFKDLVTIFLFILILSVFVFFMPNVLGDSENYVVANPMQTPPAIVPEWYLLPFYAILRSIPNKLLGVIAMLSAILMLLTMPFADLGRTRGIQFRPLSKMAFYIFIANFLILMVLGAKHVESPFIEFGQISTVIYFAHFLIIVPFVSLLENSLIELSTKKARN

SEQ ID NO: 243 - protein, 392 aa, synthetic construct
MAAKIIKKSSFFKVVLNAYLIDHSQPSNINYLWNFGSLLGLCLGIQIITGVTLAMHYNPSVAEAFNSVEHIMRDVNNGWLVRYLHSNTASAFFFLVYLHMGRGLYYGSYRAPRTLAWVIGAIILILMMGTGFLGYVLPYGQMSLWGATVITNLISAIPWIGQDIVEFIWGGFSVNNATLNRFFALHFVLPFVLAALALMHLIAVHETAGASNPLGVPGYYDRVPFAPYFLFKDLITIFIFFFVLSMSVFFMPNVLGDSENYIMANPMQTPAAIVPEWYLLPFYAILRSIPNKLLGVVAMFASLVAILALPFTDLGRTRGLQFRPLSKILFYVLLANFLILQQLGAKHVESPFIEFGQISTALYFAHFFILVPVVSVIENTLMDLYQTNNKSR

SEQ ID NO: 244 - protein, 387 aa, synthetic construct
MAARILKSHPLLRLVNSYLIDAPTPANISYLWNFGSLLAICLIIQIVTGVTLAMHYTPSVAEAFNSVEHIMRDVNNGWLIRYLHANTASAFFFLVYLHIGRGLYYGSYKSPRTLTWIIGTVILIIMMATAFLGYVLPYGQMSLWGATVITNLMSAIPWIGQDIVEFIWGGFSVNNATLNRFFALHFLLPFVLAALVLMHLIAYHDVVGSGNPLGISANYDRLPFAPYFLFKDLVTIFLFFIVLSIFVFFMPNALGDSENYVVANPMQTPPAIVPEWYLLPFYAILRSIPNKLLGVIAMFAAILALLAMPLTDLSKLRGVQFRPLSKIAFFIFVANFLILMQIGAKHVETPFIEVGQISTVLYFAHFFIIVPVVSIIENSLVELATKK

SEQ ID NO: 245 - protein, 391 aa, synthetic construct
MAARLLKSHPFLKLVNSYLIDASQPTNISYMWNFGSLLAVCLGIQIITGVTLAMHYNPSIAEAFNSVEHIMRDVNNGWLIRYLHSNTASAFFFLVYLHIGRGFYYGSYRSPRSLTWVIGTIILILMMGIGFLGYVLPYGQMSLWGATVITNLISAIPWIGQDVVEFIWGGFSVNNATLNRFFALHFVLPFVLAALVLMHLIALHDTAGSSNPLGTPGNYDRIPFAPYYLFKDLITIFIFIFVLSIFVFFMPNVLGDSENYIMANPMQTPAAIVPEWYLLPFYAILRSIPNKLLGVIAMFASLLALLALPKADLGLTKGLQFRPLSKIVFYIFVINFLILMQLGAKHVESPFIEFGQISTAIYFGYYFVIMPAVSVLENTLIDLYQANNKAI

SEQ ID NO: 246 - protein, 408 aa, synthetic construct
MAARIFKSHPLLKLVNSYIIDSPQPTNLSYLWNFGSLLALCLGIQIVTGVTLAMHYSPSITDAFDSIEHIMRDVNNGWLIRYLHANTASAFFFLVYLHMGRGIYYGSYQGSRSLTWNIGVVIFIVMIVTAFLGYVLPFGQMSLWGATVITNLLSAIPWIGQDLVEFIWGGFSVNNATLNRFFSLHYLLPFILAALVLMHLIALHDTVGSSNPLGISGNYDRLPFAPYFLFKDLVTIFLFMLGLSIFVFFMPNALGDCENYVMANPMQTPAAIVPEWYLLPFYAILRSIPDKTLGVVAMLGAILILMALPYLDKSNIRGMQFKPLSKIAFYIFIANFLVLMILGAKHVEDPFIIFGQISTTLYFSYFIIITPLFSIFDNILFDIATKKNNEIVNKYYNIVIKKDILLPE

SEQ ID NO: 247 - protein, 2818 aa, synthetic construct
MSSKAGGAFCALDPFYPVSRLTYMCQKLEISIVLTTQSNLEQASLLPGTVFVLGDDLYTESAYAGAQQPHLWSDVSPNDPVYVVFTSGSTGNPKGIIMEHRSFSSCALSAMEPLHILPQDRILHFPSYAFDISIFEILTPLVAGATVGIPSEKARRENLPHAITELGATWAFLTPTVARMYRPMQVPSLKTLCLGGEAISASDIDLWLSKNLITGYDPAECCPLASWIVDPKDHRKLLPVGAIGELVIEGPNVARGYIHDPTCSASDTPFLVTPPPWLSRFRTNFDRGSRLFPTGDLVRYDDEEAVHFMGRKDLQVKINGQRVELAEIEYHLDQHFQLLAAKVIVDTLPVSRRTILTALILPDQPLKNDEEPGKSLLMELREVHQDFQAKLASVASKLQLTLPNHVVPSVYLPIRRVPMSESGKIDRRRLKSLILSLSQEDLYGSRKPMQPGEEPASDTEKLLRSLFAQTLDLPHARIKSDSNFFQLGGDSVLAIKLLALALEQGIKSIAYQDIFNHPTLRKLSLASAPTSINGPTSSVTEETTPFSLVKDPELLIQTASEQCGVSKAEIEDIYPCTDLQQISVPLADPARRNVTLATEIQLAWAITTYTYTGCKDVIFGLVSSGRAVPVTGIESILGPTFACTPLRISIDPDEELGEALAELQYTLVEQSKFVHFGAHRIMQLGPNAAAACNFRTVLVVEADQSDRATGSWFTQHEFLSDVTSFSSHVLTLRCKLFPKSVEINAIYDNAMVDGRQMHRVLLQFERILKMVRAPESVNHKIGGLDKLSGSDWLELQAWNTRLLPPHPKGIRAQQVIQEKCRARPDATAIHAWDGEVTYGELGNRAEKLAGLVRAYISKPNQVVVLYFSRSWLTVVAQLAVLKAGAAFMTLDISQPAQHLREISATVRPVPVLTSEELYREAAELDMAAVLGIDKNALRNATAASQTCSTVESDIMYSIATSGATGKPKIVVIDHQAFMTNSRPMIDRLGITAESRVFQFCGYSLAGGCICIPSLHDRDNRLAASIVELRANWAASPSSVLQLLDPNSVPTITTLVQGGERLHQVIIDRWASHTRLLNAYGPAECSVVAFVSDSVRPDTKDPQNIGLATGGGELIIEGPILSQGYLGDTEKTNAAFVPLPDWLRAFRNGRAQGRIYRTGDIVSFLRRKDSQFKVRGQRLELLDVEYHVERCFTGAHQVVADIVPIPGTQSTVLVALILAQSAMLDQQDLETTSGGLAVLPQDSSFVADANAAELALRDKVPSYMVPDLFVPVSDFPRGVSGKVDRKAIKQHLTSLFQQDWRKYVSTGKVAPSTAMERELQIIWARVLRVEPDTFGVHDSFFRLGGDSIVSMQVAAECRAAGINITVKDMFEKRVIRKLALGRTSTQQNGPMSSSKDDESGLMEEKAPAFYPGGKLEVYMKRVQSQLGQAVERIYPCSPIQQGILMSHTRNPDHYDEIIQWRVISNAPIDTGRLHNAWLQVVSRHSILRTLFLNVSKDHFLDQVVLKHYSPDISVCNGGEEATYPPFEDFLPMHHLRVNRLSADNVTVCLRIHHALVDGISLHIIKRDLESAYQGRLAMLGEPPAYHEYISYLQNSRLQRSSEEYWKSYLQGATCCLFPALKDQTVQDRQYFGAVDLELESTLKLTQFCKKYKLAMTVVLHSVWAIIVQRYTATHEVCFGYMTSGRHAPVANVHNIVGPLFNMLVARVGLEYDQSLLSMMYKYQESFISSLDHQHQPLVETLHSIGSSAGELFNTLITVFNDQPEIQLAQQQSALTLVGDAIQSRSEYPITLNILSRADKMEMQLSYHKTWLSANFAETIAKTFRFVLQNILEQPNQLLSGLQVLDEEQMNNIYTQNRCVAPPLNGFIHDAIHQQCLRSPDSQSVCSWDGNFTYRQLDGLSSVLAEELICKGAGPEVTIPILLEKSRWTPVAMLAVLTSGSSFVLMDSSHPVARLRTISNVINPPLIIISAQTRSKATSFSINLIELGDWLAEGVHITEQYTWQNVFLNKNNAAYLVFTSGSTGKPKGAIIEHSSSFTGAECLAARLHINSASRILQFSSHAWDIPVMEILLTLRVGGCVCIPSEEERTGNLVQAANRMKANWAMLTPTVARLFKPQDFAYLQTIILAGEALSSTDITTWCDRVRLIQAYGPAECTAISTVTEPLTSSDNPRCVGRPSGCVGWVVSRNNHELLAPPGAIGELVLEGPIVGRGYFRDPERSATAFVSPPAWLLNLRGHEPSIRLYKTGDLVRQDVSSGLLTFLGRQDDQVKVRGQRVEPGEVETQVSQVFPESQVIILVVKRSDTAILVALILRKDEAGSCSTKTGNVISPPSPAFAKLVRTAFSRLRETMPTYMIPSFMLPLAQLPNASTGKADRKLLQDYVASLSNDELEEYITANASRRVASTAVEAQLQELVGQVLHKPSHTIPLDEDLFRIGMDSLTAMTFATAARGCGWEVSVPIIFQHSRLSDLALLLDRGQHIKQGLLKETPIPNPLASFLPEICTKWHLQENQITHIVPTTYYQQMAIASDHYVFVGLHFSRPIASEALKSAASRLVKQHSILRTVFIPFQNAYVQLTLRDFDLPSLEIQSNEAELSTDVENFCREAAKNAAEFGVPHTKLVLILNKEGGCLSALLRLQRAQVDGVALMRMIADWRFAMEEEPSLPVPTLEYADFVLGRIAQNTPEVFGMWRDQQHIHTMTGATADWGDVVAKSTTWPSDTEFGSVLHYLSAPASPAYDFPGDICGRFQLYDYKMAHTCPLVTCIVFPGRMDPSLKELKIIVTSAGCGQEMTDQLLSMFQIMLHQANNHSDKLISDLLRVES

SEQ ID NO: 248 - protein, 3555 aa, synthetic construct
MSSSITIQIGNYNSMAVNQALPINHNKSNGDLNDDKDTPDQDVSSDSGVSTANNYQPCILTGFTPAPTRSTPPKVQSASVNGKESISKLKTFLEEYDVSAAAAHKGAWAIVLGTYLGRSYVSLDYGVINHQALAIEATCNDRRTIEIIPCTIRFKESDTLLDIIRQDTICTHAGSYQKFAIPPVEIENRCNTCVIHWTGESFPENIQSSALMRVLVEDEQLAKYDCVVNFTSEVQCIITYRDNFMSPDQADHLAATLLVVLNHITDAPRQSVADIDIFSSLDHQKLSQWNQKTPVASEVCVHHLIEQSRQSRPNIQAVVSWDGSLTYDEIDRLSSNLAHRLREAGVRPGVFVAMCLDRCKWTPVGMLGVMKAGGAFCAFDPYYPVSRLTYMCRKLQIPIILTTEDNFQQASLLPGTVFVLGDDLYTESPYPGAQQPHFRSEVSPNDPVYVVFTSGSTGNPKGILMEHRSFSSCALSCMEPLHIMPQDRILHFSSYSFDLSIFEILATLVAGATIAIPSEKARRENLPHTITELGATWAFLTPTVARMYRPKQVPSLKTLCLGGEAISASDVELWSSKNLITGYNPAECCPLGISGPANQYMPRNLGLAFSSQASWIVDLKDYKKLLPVGAMGELVIEGPSVARGYIHDPTCSASDTPFLVTPPPWLSRFRANLNQGTRLYLTGDLARYNDDGTVNFMGRKDLQVKIHGQRVELAEIEYHLDQHFQLLAAKVIVEILPVSGRTILAALILPDQRLEKDEEPQNSLLMELSEVNQDFQAKLASAASKLELALPKHMVPNIYLLIRHVPMSESGKIDRRRLKSLVLSLSPEDLYGCRKPTGPDKEPASDMEKLLRSLFAQILDVPHTRINLDSNFFQLGGDSVLAIKLLALALEQGIVSMTYQGIFNHPTLRDLSLASTPTSSSVPTPLVAEETTPFSLVKDPELLIQIASEQCVVSKADIEDIYPCIPLQQSTMASTAHNSSAYVAVLSFTLSGGIDRDRLKRAWHIALNGHAMLRTRLIQADTGDCYQVVIRGASCWIENRELSDGDTTNLCRASFGLGLPLIQAHLAKDRLYVAMHHALYDGWSLPVLIDELDLAYRELSVRRLPSLKHYVKYMMDSAASAALFWRAELEDAEPVHFPAPSRLDYKPHPCASLTVSVPLVDSPRRNVTLATEIQLAWAVTTYAYTGCRDVIFGLMSSGRAAPVPGIESVLGPTFACTPLRISIDPDEDLGEALEELQYTLVEQSKFVHFGAHKIMQLGQNAAAACNFQTVLAVEADRPDSVTRSWFTQYKFLSDVTSFSSHALTLRCKLFPKGVEINAIYDNAIVDDRQMRRILSQFEHVLTQVHATESVNCKIGGLVKLSGSDWLEVQAWNNTLLPPHPRGIRAQQMIQEQCRARPHAAAIHAWDGEVTYGELENRAEKLADLVRTYISKPGQFVVLYFSKSWLTVVAQLAVLKAGAAFMTLDISQPAQHLREISVAVRPVLVLTSGELYSEAAELEMATVLGIDKDALSNGPVASLTCSTAESDIMYGIATSGTTGKPKIVLVDHQAFMTNSRPMITRLGITAESRVFQFSGYSFDLMIVEHFLTLLAGGCICIPSLHDRDNRLAASIIELRANWAASPSSVLQLLDPTSVPTITTLMQGGERLHQVIIDRWAPHVRLLNAYGPAECSVVAFVSDPVRPDTKDPQNLGFATGGVGWVVNADHPDSFPVPIGAEGELIIEGPILSQGYLDDAEKTSAAFVPLPDWLKAFRDGRAQGRIYRTGDIVRQNSDGSISFLRRKDSQVKVRGQRLELLDVEHHVERCFAGAYQVVADIVQIPGTQSTMLVALILTQSAILDQQDLEKSSGGLAILPHDPSFIADANAAELALRDRVPGYMVPDLFVPVSEFPRELSGKVGRKAIKQHLASLFQQDWRKYVSTGKVAPSTAMERELQSIWAHVLHIEPDSFGVHDSFFRLGGDSISSMRVATECSAVGINITVKDMFEKRTIRKLALGRTTTRQNGSSSRDDNSGMMEEKAPTFYPDGKLDVYMERVQSQLGQAVEGIYPCSPVQQGILMSHMRNPDHYDEIVRWRVASNAPIDTSRLQNAWLQVVSRHSILRTLFLNVSDDHFLDQVVLKHYSPDISVCNGGEEMKFPPFKDFLPLHHLRINHLSADNVSVCLHIHHALVDGTSLQIIKRDLEFGYQGRLAVLDEPPAYHEYISYLRKMCLKKSPAEYWKSYLQGATCCLFPALKDRAAQDRQYFGAVDLELGPTVKLTQFCEKYKLAMTVVLHIVWAIIVQRYTATHEVCFGYMTSGRHISVANVHNIVGPLFNVLVSRVGLAYEQSLLSIMYQYQESFVSSLDHQHQPLAETLHSIGSSAGDLFNTLMTVFNDQPEIQLDQQQSGLSLIGDAIQSRSEYPITLNIFNRADKMQMQLSYHTSWLSANFAETIANAFRFVLQKILEQPNQLLIGLRILDEKQMDNSYAQNRCVAPPLNDFIHDEIHQQCLRFPDSESVCSWDGNLTYRQLDGLSSVLAEELIRKGAGPEVTIPILLEKSRWTPVAMLAVLKSGSSFALMDSSHPVARLRTIFTTINPPVIIVSAQTRSKATSFSKNMIELGDWLTEGVHITEQHNWQNVFLKKDNAAYLVFTSGSTGEPKGAIIEHSSLSQAAECLAARSHINSASRVLQFSSHAWDVSVMEVLLTLRVGGCVCIPSEEERTGNLVQTANRMKVNWAILAPTVARLFRPQDFTHLRTLVLAGEALSSTDITAWCERVRLIQAYGPAECAVISITTEPLTPSDNPRCIGRPSGCVAWVVNRDSHDLLAPPGAIGELVLEGPIVGRGYFRDPERSAAAFVNPPAWLLNLRGHESSIRLYKTGDLVRQDVSSGLLTFIGRKDEQVKIRGQRVEPGEVETQVSQVFPGSQVLVLVVRRSGTTILVALILQRNENSSCPTKTGNVLLPPSSAFAESARAAFSRLRDTMPTYMLPSFILPLAHLPNASTGKADRKLLQDCVASLSDDELEEYTAANTSRRIASTAAEAQLQELVGQVLHKPPHTIPLDEDLFRFGMDSLTAMTFATAARRYGWEVSVPIIFQHSRLSDLALHVNRGGQHNKHGPLQRAPISNPLVSSLPEICIKWHLQEDQITHIVPTTYYQQMALAADHVAFIGLHFSRPIASEALKSAASRLVKQHSIFRTVFVPFQDAFVQLTLRDFDLPSQEIQSNEAELSRDIELFCREAAKKAVGFGVPSTKLVLVLNKEGGCLSALLRLQRAQFDGIAVMRTMADWRSAMEEEPSLSAPTIDYADFVLERIAQNTPEVFSMWRDLLEGSSMTYPFPQHEYIRMTDRSRTERLVESSCDIPLPEPARGFTMATVAKAAWAICLARQAQTQDVLFLQLVRNRHLVLDGIDKMVGCCINYIPVRVPLQPDWTASDLLHWIQQQHLHTMTGDTVDWDDMVAKCTTWASDTEFGSVLHYLSAPASPAYDFPGEICGRFQLYDDKMLHTCPSVTCIVNPGRTDPSPRELKIIVTSTVCSQEIADRLLSMFQTMLHLINNHPNALVLDLVRARDPAIGGAKLCEL

SEQ ID NO: 249 - protein, 1305 aa, synthetic construct
MAAATLEDWKAYLEGYSPGNFPKLSGIGDVDFATSTSVTATLRDPYCKLQSFSNEHAVDTATVFCVGWGVVLQAYTGQDLICFGISKPSISPCRIDFTSKAVIRDILATFQNGFDKEPLHLEIPMSRFLQNDKHFVTTFFNTCILYPNREVRVQNGVSDTSADCNSLTELDVVIRYDDEEGIVRIHMTYKTSLLQKAQALTVINALERAVSEIINGKDVVEQLCLLSTDDRHHIFSQKVRLPDSSDDQIDKLIRDRCNERPSALAVCAWDGNFSYMELIELSTALAQHLAVQGTGPEIFVPVLLEKSRWAIVAMLGVMKAGGAFVLLDPSHPEERLRDICKQFSTKLIISSSQHAQLAASLGRQVVMVQDGEEIGWNRGGYHFVKSPVTPNSALYAVFTSGSTGTPKGVVNNHSSFLAAMPTYLDSVMLNKDSRVFQFASYAFDVTIFDTLMTLVAGGCLCVPSNNDRSSDLTNAVQRFAATHLSLTPTVARVLDPQDFPTLTTIVLGGECLIPSEIFRWADHVRVVHLYGATECAIMSVQCTSGTPPNSRTINYDTGCACWVVNPRNPEKLQPVGAIGELIVEGPVVSRGYLSDPTRTSETFIEAPTWLRELRGKRSTVYKSSELVRIAGDGTIQIIGRKNMQIKLRGQRIELGEVEHHLKLAIPYVADCIAELVTVPDSSRPPMLMGFIRSAFDVGVQSIATGHNATASNVFAEPTSAFRSQVPSILSSVSKSLPSYMVPSTIIPLRTLPLTSTDKVNRKLLRELAGGLSREEFGKYLPHARIYRAPTNDMEEALQKYFASAMNMSVEQVGVDDNFFSLGGDSLTAMKLVAMARKARLSLTVQNVFDHPQLSELARNAQGVSDENRDQPQPYALIGDNKRDVIRDAARQCQLPSRAIEDIYPCTQVQIGLMTETMRNPGLFIANIALPLRGDIDIDRLRQAWTAVAKANPILRTRMILSSSHGMLQAVVREDIRWIVSDVPSPQDMVGFGKPLVQLVLSLIPEGVPGQNKLLITIHHAIYDGYTLPLLFEQVETAYSGGVLASRPVSPFIRYLAQVPNGTQYWPALFEGLETPPFPPLPYESHLPSPDSQETLNVSVSSPSAKQYTPNTYLHLAWAITQAHQQSLRDVFYGAVVSGRGAPVDDIELMTIPTVATIPCRVSLDAGSTVQQTLRRIQEVATKGIPYEQLGLIEIRRLGKYTAHGCSFQTVLVLQPAVQRGVSDLFEIPEADVNYRQDATYAVNLFCELSQQQLIVTAFYDERIVPRSRMQEILETLGRTLKAIHTAPTSVIGDILGTLVTSRGST

SEQ ID NO: 250 - protein, 3586 aa, synthetic construct
MAASIPIPEKLQDLTAVKSPSAFGNSIESINGDKNKSERHTASSSAVSTSEIGHPCLLTNFKLAVACSGPAITKVATVNDKDSGSKLKNVINGKDISASEVFKGAWSVVLGTYLAKSHVSLDYGVMKPKGLGPETSCNARVPSENSEMSTSSFLLRANDTLLDIIRQNSMCAHTELRQKSSLDDVESPKRCNTCVIYWPEISCSEQLQIDAWMTILEENDQLTQYDCMIHFASDMRCMLSYRDQFMSENQARHLAATMRVVLSSIASAPQQSLADVDVCSSLDYQTLSRWNLKAPIVSEVCVHDLIEKSCCSRPNSQAVVSWDGCLTYNEMDRLSSHLAQRLRDAGVEPGVFVALCLDRCKWAVIGIVAVMKAGGAFCALDPSYPVSRLKEMCRDLGITIVLTVKSNIQHASPLASKVFALDDDVYFESALSSAHESASWVSVSPHDPVYAVFTSGSTGKPKGIIMEHASFSACALSSVKPLQIADQDRVLHFASYAFDASVIEILAPLIAGATVAIPSERARLEDLPRAMTDLKATWAFLTPTVARLYRPEQMPTLKTLCLGGEAVNASDTRSWSSKNLISGYNPAECCPLGISGPLNDRMPRSLGSTFASQTAWIVDPKDHEKLLPAGAIGELAIEGPVVARGYIHDVTCSDPSTPFVVKLPPWLRRFRATANRGNRIYLTGDLARLDCDDGSVHYLGRKDDQVKIHGQRVELAEIEHHLEQHFVSLATKAVVMLLRPISGRTVLAALIMPHQRLQHGNKSLESLLMEPGDVSQDFRANLASAASKLRLALPSHMVPSVYLPIRHFSTTKSGKIDRGHLQSLLLSLSPENLYGCEETTHRGEEPKSDREKLLQALFAQSLDLPCTRIDLDSNFFQLGGDSLSAMRLLALALEEGISSIAYQDIFSHPTLREIVIVSTSATSREPLYSETVETPPFSLIKDPEMLIQIASEQCGSGVGKADIEDIYPCTHLQQSLMASTAHNPNAYVAILAFKLKSGVDRTRLERAWHIACSGHAILRTRLVQTDTGDCYQVVVKKPPHWTETNEVSDDGSTNSLLRTSFGLGRPLIQLHLTTDQLFVAMHHALYDGWSLPMLIGELDLAYRELSVRRLPCLKNYVKYAMDSADAAASFWQAELQDADPVHFPAPSSLDYKPQPCAAMTISVPLVNSPRRNVTLATEIQFAWAMTVYTYTGCKDVIFGLISSGRAAPVAQIESILGPTFACTPLRVSIDPQGKLGEALDDLQYTIVEQSMFVHFGAQAIRQLGPNAAAACNFQTVLAVEADGPETGEEEGSWFTRYDFLSDVASFSSYALTLRCKLSTRGVEINAVYDKLMVDERQMGRILAQFEHILTQIHSNETVHDDIGGLDKLSVSDWRQLQAWNSNLPPAHPKGLGAHQAIQAKCQAQPDATAIDAWDGCVTYGELERRAEKLAGLVRSHVSKPDQVVVLYFSKSWLTVVAQLAVLKAGAAFITLEISQPVHYLQRVISALGPVLVLTSEDLFSAAEDLQDNAVPVMAVDKDDLSDATARTSQASSSACTVECDLMYIIATSGTTGMPKIVMTDHQAFMTNASPLMNGLGITSDSRVFQFCGYSFDLLIVEHFLTLLAGGCICIPSLHNRNNRFAASIVELEANWVGSPSSVLQLLDPQTVPTVKTIMQAGERLQQGLVDRWASHVRLINAYGPAECSVGALARDTVRPDTDDVQNLGFATGSVCWIVNAETSEKLLPVPIGAEGELIIEGHTLSRGYLGDADKTNASFLRLPNWLRDFRADRGQSQGHRVYLTGDIVRQNSDGSMSFVRRKDAQVKIRGQRVELTDVEHQVERCFIGAHQVVTDIVQIPNSQSSILVALVLTKDAMTNHKQQESLLDQKSAGGLSILAPTSSFTANANAAETALQDRMPAYMVPDLFVPVSDLPREASGKIGRKAIKQYLASLTQQDWSRYSSTRKVPPSNATEHEISAIWARVLQIEPHTFGVHDSFFRLGGDSISGMQVAAACGAAGISVTVKDMFEYRTIRKLALARGETQQLTVGTTSTVSNASGIRQKKALHPFYPEGRLEVYMERMQSRLGQAIERIYPCSPIQQGILMSHARNPHHYDEVIQWKVAGDVSCDISRMQRAWREVVSRHGILRTLFLQVSEDSFLDQVVLKNYSPDISVCTNEEDVEPYRPFEDSVPMHHLLVFQRSADDVTVYLRIHHALVDGLSLHIIRRDLELAYQGRLDELAQPPGYHEYISYLQEKRSRKSLQEYWSSYLQGATGSLFPAVQDEPASDGQYFGAVEIELGSIAKLTQFCEEHKLGVTVVLHVVWAIIVQRYAATDEVCFGYMTSGRHVPVTNVENVVGPLFNMLIGRVKLAYHLSVLSTMYAYQENFINSLDHQHQSLVETLHSIGSSAGDLFNTLITVVNDQPEDHVSQSALRLVGDSVQSRSEYPITLNILNHADKIKMQLSYHTSLLSGVSANTIAKAFRFVLQRTLEQPHELLRALPVLDEDQMNNEFQKNRSVPPQVEELIHDTIHQQCIRCPDSPSVCAWDGNFTYRQLDDLSSALSEEIVRKGAGPEVTIPIVLEKTRWTPVAMLAVLKSGSSFVLMDSTHPAARLGAIIQDVGHPVIIVSAQTRSKVATFSTDVVEVGDWLAREILVAKQQITRQNGLLQATNAAYLVFTSGSTGKPKGAIVEHASLSTAAKYMASRLHIDSASRVLQFSSHAWDIPVTEVLVTLRMGGCVCVPSEEERTGNLAKASERMKVNWALWTPTVARLFKPEEFPHLETLVFAGEALSAADLETWCDRVRLIQGYGPAECSLISTVTDPLTRSDNPRCIGLPSGCVAWVVNRDTHELLAPPGAIGELVLEGPIVGRGYLGDPERAASAFISPPAWLMKLRGSGSSIRLYKTGDLVRQHVSSGLLTFVGRNDDQVKVRGQRVEPGEVEGQVAQVFPGSQVIVLVVKRSAGAVLAALVLQNGEDRSSAGETANLFPPPSLAFAALAKAAFSKLRETMPTYMIPSIILPLSYLPKAATGKADRNLLRDRVASLSDEEIEAYVAASVSHRPASTAMEAELQQLVGQVLQRPLHSISLDEDLFRLGMDSLTAMTIASAARRRGWEVSVPIIFQHSRLSDLARIVEQGQHGTSSRSQLEEARAILNKRLVSLLPEICTKWDLREDQITHIAPTTYYQHMALASDHEAFFGLYFSKPMASEALKAAASRVVKLHSILRTAFVPLEDTYVQLTLCDFDLPSQEIQTNQAEVSAAMELFCRDAADKTAGFGVPVTKLILMLDRQGDCLSLLLRLQRAQFDGVSVMRIMADWRSALEHASCSWEPAPSLDYADFALGRVAQNTPDVFGMWRDVLQGSSMTYLIPQEKYISMTDRAHAERLVTSSCDIPLPEPAPGYTMATVAKAAWAICLARETESEDLLFLQLVRNRHLALDGIDKMVGCSLNYVPVRVPLRRDWKISDLLHWLHQQHIRTMAGDTADWPDVVAKSTTWSSDTEFGSVIHYLSAPAAPVYHFPGDTVAQFQLYDEKMTHTCPLVTCVEFPGPAEDSGRQMKILVTSAVGGQDMVDRLLAVFRSLLCEANAQLDQPLSNILQGLRDGDDATGKAR

SEQ ID NO: 251 - protein, 1305 aa, synthetic construct
MAAATPEKWRKYLEDYIPCSFPTFLDNEGADSKSFASVLVRLEHPYGRLMSFSNNCGVDVAVVLNVAWGIVLQAYTGQDATCFAVIAESNLNIRPCRIRFTSDKVVSDILSACQSPCGEKTGDHDIPASHLSQDGGFLASEFFNTCIWGPMQGSQMPSETQAADMNRNALNLFDLVTRVEVDKSITRITLTYKRGLMREHQALAVAKAMERAISEIISGKERLDQFCLLTSEDRRQMSLWNMNLSDNSDARIETLIHEWCRWTPSAVAVCGWDGDFSYKELNELSTGVKHDLRHLGIGPEVFVPILFEKSRWAVIAMLGVMKAGGAFILLDPAHPPKRLRSICDKVSARLVVSSVQQADLAAGLAGHVVIVGGEVATAGMAQHVGEHDDSMDCIAAPHNALYAVFTSGSTGTPKGVVNSHSSFLAAMPVYLKALELDNNSRVFQFASYAFDVTIFDALMTLVAGGCVCVPSNADRSSDLTSAIQHFGTTHLSVTPTVARILDPQDFPSLKTIVLGGELSASDELLKWVNNVRVIRLYGASECTVMSIQCTSGPASSIKTINYETGNCCWVVNPQNHEQLRPLGAVGELLVEGAVVGRGYLDDASQTSETFIEAPAWLQELRQGSSTVYKSGDLVRIAADKSVQFVCRKSTQVKLRGQRIELGEVEHHVRLAIPSATECVVELITNPDASRPPMLMAFVLSDTDASTSSITARRNATSDAVFAEPSASFRSQIASITSKLRDALPSYMVPSVILPLRIMPLTSTDKINRKLLGQLAAALSREDLQLYQAQQTTYRAPSNDIEEAFQRFFAQVLGLSLDQIGADDHFFSLGGDSLTAMRLAAMARKAKFDLTVQNVFDHPELSELARHTKLVADESQEFPPPFTLVAGSKQGIVRDAARQCRLPSRVIEDVYPCTPLQKGLLAETMRDAAAFVAKIEVPLPRDVDLDRLRHAWAAVAKANPILRTRMIFSPSYGMLQVVVREDIPWIESDDVESQELVAVGRSLVQLILRRRPSTALFLHIHHAVYDGYSLPLMFAQLNNAYHGETLAFRPASAFIRYLATMPDATDYWQSMCQGLESPSFPALPHSSHRPHPDSKATHTVCVASPQAREYTPNTHVRLAWAITQAHEQGLLDVFYGTVVSGRNAPVDQIESMLIPTVATVPCRISLDVDSPVRKILHRIQDVATRGIPFEQIGLAEISHLGKDAAHACSFQTLLLMQPTAVEQNENDFFNTSTSDANYRADATYAINLFCTLENQDLSVTALYDGNIVSTDTMQRLLQNLGKSMQEIHAAPRTLIGDILKSLHSRL

SEQ ID NO: 252 - protein, 1794 aa, synthetic construct
MSSNSIKLKFNCKDSLWQEHIGAGDFSFAAIFLTTWGIVLRAYLDTDDVFFEYGLSRCEPVSQRDGIDELVPDRPFNLKFDGSMTVKDTIRRADSVVYGTPLVQDGLEHETHRETDPKRETFYSTCMLFLDGPKSPIDADVVEGTILDDIEFEMKPVVNYDMVVYVLSGKVCYLSYNVTRVSDDQAAHVAATFETVAKCIADVPHRLVQEVESLSQLDVDRLKTWNAYQPIAVETCYQDLFRQRCDLHPNSPAVIAWDGSFTYDELDHFSSLLATRLQAAGIGPDVFVTICATRCRWIPVAMLGIIKARGAFCALDLSHPLDRLKDICDALKSTITITTPTDSNIARKLASTVIVIGGDAPVESDRITPMNDRPKPINGHPTNALYSVFTSGSSGKPKGVVVEHRSFVSSALASIQPLDIRPHDRVLHFSAYAFDISVFEVLTPLISGATIAIPSEKRRKESLTHAVQELGATWALLTPTVARLYDPDEFPSLRTLALGGELAQASDIALWQSKNVVVIYNPAECCPIGVSGPACPADGKFLGWSHTCQRAWIVDPRDHDKLPPIGAVGELLIEGPVVARCYAHDPNFSSPDSPFIQSTPSWMLRLRSNTPSGTRLYRTGDLARYGSDASLYYMGRKDSQIKIRGQRTEPGEIESNLHSILSKDKLGVAIVVLELRGSSKIIAFVSKDTGGLGGDSNTVGQLRIEAATEETDVCITKATSKLHSIMPAYMVPSAFLSVNYIPISRSGKIDRTRLKSFALSLPQETLLRVNNGLETGDLPESNEEHRLQRMYSLVLGISRDKVGMESDFFRLGGDSLQAMKLLALAPKEGLTDISYEDIFRYPRLKDLARKASQSVTIKKDGFGENSSVIHPFSLVIDGQSLIDMAAKQCDIERDSIEDIYPCTPMQASIISLAVKGKIMPFLTFGLALRDHVDTKRVKDTWHAAYRANSLLRTRIIVCAETGQLYQVVVGGDIFWDDDECGNFAQPKSGPSASIGGPLVRMKLVEGQLSIAIHRALYDNWSIRQLLNDISGAYNGLTLPSRPSFNCYVSYAARSLEAASSFCNAELGDSDLDAAKYPEPVSQNSHTNFRAWLGIRVFTCQKESIDVLASEFQLAWAMIAYARTNKKDVVFGVLSSGRSNASKDTKEIMGPIATVTPLRVTIDGTQDVGGALEELQYRQEEQAMYTHLGLRRIGQLGRNAAAACQIQTVLIVEPDLPDLRGVWFSNDATLPNHSDADASNYRLTIKCVVGPDCTDIFAIFDHQSLPIMEVKEILSQFEHILGQIHGKEASQLSVASIDTVNFKDWDTLHKLTEMPSVCRNGLLLSDPTILPQGQMKTFSAVEEAAAHCAFQDSLQEASIARDAKMQPKEPLSSADLISEINRYDLAIMRSRPSPESILLSELALTGDSHSSGTHTVFVTGANGFIGTQILRHCLEDPTIDRVIALVRGSSANEARSRTEESARRAQWWSDCHSQKLEVWPGDLAMPHLGLNETHWRRLADRTTINAIIHNGASVHWLKRYADLEATNVGATAQLLQLAVANPRLGFVYVSSGRYTDPNAESEEPAAANVAATAMPYSQTKFVAESLIRRTAARLPHGQTQVRIISLGLVIGDPLTGVVNADDYLWRLIATCVQAGEFNSSAGSEWMPISDVTSTALAIVQTALNPAGVPATIKPITGGLMWSEIWDLVTDMGYDMEPRPESEWMATVRRDLEREQERHPLWTLSHLVESRSQLNTDAGAGPAWADAWRGDEATTRNLKTAFRRSLRFLGEVGFLPGQKGQNTDGEVNGRAFTRAW

SEQ ID NO: 253 - protein, 3591 aa, synthetic construct
MSSGSTTVEPVGSTYARPKPLGNLDHRIEQLLGSESVLNESPGHSPNAYGIPYTARNLTLEDVDHCKSLTNKDLSLEFWKSHLQTAKPCILTRLKPGTGSSILSSRQSVKVDLGGVNTTCRNLFDSTGISLMTVFKVAWGMVLSTYTGSDHVCFGYGAARPSMSVDGEQTTIKPLIKMLPCDMLFKGGTTLLGTVHKAESDFICTIPYQSVSIAEIHHGIGTESGALFNTSIIFWPQEDFSDNPITEVLSQKVTETESLTECDIIMHVDSKQQCYLSYWDYFLSDEQANHLAGALNVAMKSIFESPHRPVGQVEMFSYLDQQKLLQWNGQAPIASESCLGDLIGQNCHSRPDSLAVDSWDGFFTYQELDRLSSTLANKLRAAGVGPDVFVTACFDRCKWMPVAMLGIIKAGGAICALDPSYPLGRLTGMCQLLKSTVVLTTANNAQQAEQLALTTIVLGNDLWTGDSYDEQERQAPLSNVCPSNALYAVFTSGSTGKPKGVVVEHRSFSSCALASLKPLDIRPHDRVLHISSYAFDISIFETLATLTAGASVAIPSEKARREDLPGAMRELQATWAFLTPTVARMYQPKDFPSLRTLCLGGEAIHTSDIGLWASKNLVTGYNPAESCPLGISGPADKSAASFLGWSFSSQASWIVDPRDYQKLAPIGAVGELMIEGPAVARGYIHDLTCSHPDSPFVLPPPQWLSRFRASTSQDTRLYRTGDLVQYGSNGSVHFIGRKDLQVKVHGQRVELSEIEFQLHKTLLPLDCKVVVDAVTFTGHTSVIAFITAAEHSDLENEDDAVRSPDVKVITQDFEIRAADAATKLQSILPRHMVPTIYLPVGHIPMSRSGKIDRQKLRSLALSLPRETLYHIGRQPGPGEIVATDVERRLQLLFAQVLDLSPEKIKADSDFFRLGGDSIYAMKLLALAPQQGLRDLTYEEIFRHPKLRDLAAASSSFSNIFLAIPEDLGPAPFSLARDADSLTKIASEQCGVAVGDIEDIYPCTSLQESLIASTAHDQDAYVGVQSFTLNDDIDTTRLKMAWKMTSEGHPILRTRIIQTDSGAPYQAVIRGPLSWSEESSSDDLPPQFQPSIGLGTPLVQLCLTNSRLLVAMHHALYDGWSLPLLLAEVDQAYRQLSVRQLPPFNRYVKHVTETVDSAASFWKAELQDADPVDFPPLPHLNYKPEPRALLTKSITLTAHTNSQHNVTRATELQLAWALTSHTYTNSQDVVFGIISSGRGAPVAGIERMLGPTFASTPLRVPLDPAQDVREALEELQYRLAEQTKYVQIGLQRIRQQGPNAAAACSFQTMLVVEPNQPVKTQSAWFSRHEFLSELTRFSSHSLTLRCKLLAGSVEVTAIYDQLVVPDAQMQRILSQFQHILTQIQGIGSRDTTIGDIDRLSSGDWNELQAWNSTLPPGLELSVHQMVQAKAQMQPEALAIHSWDGDLTYKELEDYAKGLANRLHALGVRPNTFVAIYLQKSLWVVVAQLAVLIAGAAFTTLETSQPINRLRDVCRTVQPTVVLTSDKLRLSGADLEVPAPLLVINQQLLLEEPGSHSRPLENHTIMASDAMYSIATSGTTGKPKVVVIEHQAFLANSKHLIDRWGFTADSRVLQFAGYSFDAMIVEHFITLLAGGCICIPSSFDRDNRLATCIVEMRVNWAMLTSSVIPLFTPAAVPTLQTLVQAGEPMHQGITDCWASHVQLFNAYGPTECSVISTTSNVINPDARNPRNIGFTTGGVCWIVDPENPESPPVPIGAEGELIIEGAILARGYLGDRVRTAAAFTPRPGWLDDFRGSSGDNRIYRTGDIVRYDPDGSISYVRRKDSQVKLRGQRVELLDVEHHLQNCFPGALQVVADIVTVPNTQSSALVALVLATPTSSSSVAIESCSNDDQATTAHGLLLLANNPQFLIDASAAELALQDRVPSYMVPSLFIPTSRFPRDVSGKVNRGEITRSLAALSRQEWDGYVSTNRVAPNSGLERELQKIWARILNIPPDTIGVHDSFFRLGGDSITCMQVAAQCSRTGILITVKDIFKRRTIEKLAAAAVVVQCPESSTTERVNTAEAKFSFYGPGQLEEYMIQIQPQLGEGQVVEDIYPCSPIQRGILMSHARNSGNYEEVIQWKVISRAPVDVCRLRDAWAQVVDRHAVLRTLFLHVCKENYLDQVVLRSHSPMVLVYNEGEEPVNPVSTGCSQPMHHLRVKRSSAGGITVRLHINHALVDGASLFIIRRDLAMAYEGRLASSRASSPYRDYIAYLQNCHSQIQSNEYWQSYMEGTAPCLFPTLKNAGAQDSQQPFEAFTLQLGATADLNQFCENHRLALTSVLHVVWAMVVQRYTAMDEVCFGYMTSGRHVPVAGVQDIVGPLFNMLVARVGLPHDATLLSVMQKYHDNFLISLDHQHQSLAETLHSIGSASGELFNTLVSIFNDQREGEPAHKSSAVTLVGDDIHSRSEYAITLNVLMLADQVHIQLSYHTSLLSDNYARMIAETFRHVLATVLGQPQLRLNEIEMLDEEHRSGLYGRNHAAVPSFDSCIHYTIHQRCLESPDSPAICAWDGDFSYRRLDQLSSSLAEELIGHGVGVEMTIPVLLEKTCWTPVAMLAVLKSGASFVLMDASHPLGRLQTICEAINPPVILASPQTRSKAVGLTSHVIEVTNRLLEQEQAEQQQTWPRVVTKGSNAAYVVFTSGSTGKPKGAIVDHSCLATAAGHLPSRMYINSASRVLQFSSHAWDIPVTDVLLTLRVGGCVCIPSDEERIGNLAQVANRMMVNWALLTPTVARLVKPEDFTHLQTLVLAGEAVSSTDLTTWHDKVRLIQGYGPAECSLVSTVSEPLTPSDNPRNIGQPNGCVAWVVHRDNHHLLAPSGAIGELVLEGPIVSRGYINDPERSAAVFVDPPTWLTRLRGGHSPTRLYKTGDLVSAGLDGCLSFVGRKDDQVKIRGQRVELGEVEALASQAFPGSHVVVETVKDLSSTILVAFILQKETAHAQPSSISSLLHPPSPLFRELISAASCSLRETMPSFMIPTVFLPLAHLPKAPTGKTDRKFLRGHVASLSRMELEAYSIVDATGRAPSTPLETRLQELVGRVLHRSPESIPLDEDLFTFGLDSLTAMTLATLVREDGLAISVPTIFQRPRLSELAVVLNQEQQIKQGQFLAPPPNALMASMDELCAQWQLDRSQVVNIAPTTYYQRGSLASHHTNFIALHFSQPLDPIAFRNAVVGLVQKHAILRTAFVPFRETFVQLILRDFDLPVQEIRTDEDDPSVVAESFCREADRVPVSFGTPITQLYMILGRAGDRLSAVLRLQRAQYDGVAVSCMIADLRSAFDEAPSSALPTLEYADYVISRRAHSSPPVFQVWRELLQGSSMTYLVPPTEYIRSTDRSRTELLVTSSCDIPMPDTKGGITMATVIKTAWALCLARQTQSQDVVFAQLVRNRHLAIAGIDRTVGPCINYVPVRASLNLDWTAKEFLHWVQRQHIRTMTCDMADWDDLVIESTSWPRDTELGSAVHYLSAPVASDYTFAGDVPCQFQMYDFKMVHTYPMVTCLPFPSVGDSSLTVLKIILTSAVFGQGVADRLLSLFRDMVHRLTTYPESLVSELLIIG

SEQ ID NO: 254 - protein, 1353 aa, synthetic construct
MSSQSQVRLPSNGNPALSFPQNEIGGPVHWKTYLEGCSPCSFPSLRTVCPSQSWPASVSITIGDSIFRRLEHFSDKTGISVATVFRASWGLVLRIYTGQDSVCFGDMTTAPGGIDAIGCVGVCRVELSDTAVISKVLQRIQAASANKLAIPHVPLSDAVLSKCMPSASLFNTCMLISGGGERPEKTSSTFEKVELGYDDQYDIIVRGSVEDSGASAALSYRTSFLSEEQATSIANTFERAVSDLIGIENRIGQICFLSDLDKSQIYTWNKDPPLRAHSCVDTLIHERCLSQPTASAVNAWDGELSYEELNHLSSKLSRHLVTLGVGAEVFVPLCFEKSRWTTVAMLAVIKAGGAFVLLDPSHPAERLLSICQKVSARLIVASAQHAKLAEDLVTSIVEVGDDKADWLTDKGIKAQTQTRTRRSAAPGDALYAVFTSGSTGTPKGVIIEHGSFHAAVFPYTEAVGLNQESRVFQFSSYAFDVTIFDTLMTLISGGCVCVPSNTERWSDVANAIQRFRVTHSSLTPTVARILDPKDVLTLRTLVLGGEKLVTSDITKWVDQVRLVHLYGASECPIMSIQSMTGVASDFQTTDHATGSNCWIVDPNNHDRLVPIGTIGELVIEGTIVGRGYLDDPEKSSATFIRPPGWLCQVRGSGYHSAVYKSGDLVQYTADGSLRYIGRKDTQVKLRGQRVELGEVEHHVKLTFPNATDVVVELVVTIHASSSRAPILVAFVLISHEADPDPESIRIGRGEGLSQILSEPTDRFCSQIPIVQSQLQQSLPSYMVPGIFLPLMTLPLTSTDKINRKLLRELAGALSREELESYQPSTGPVRAPQTTTEKLLQQYFARVLNIPVEQVGADDHFFQRGGDSLTAMKLVAMARKDKHKLTVQDIFDSPRLSALACVVRSGKVDGNKEPPLEPFSLVNKHRDIIRAAAQQCQLPVRVIEDVYPCTPLQRGLISETLRDSKAFIAHLAVSLPPDIDLQRLQEAWTTVANANPILRTRMVLSASHGLLQVVVREDIRWIVSKNAEAQDFFVGVGKPLVQLVLCCHREGNEVPVQLLLMVHHAVYDGYTLPLIFEQVKAAYNGGTLAPRPAVPFIRYVQSIPDGTGYWNSLMANLQTPIFPALPSKSYQPLPNAIMRHTIITPGSHRQYTPSTYVRLAWAITQAYHQGTCDIFFGTEVSGRNAPVTDIELMTIPTVATIPCRVTLDFQSLVQSALRKIQDDAISGIPFEQLGLPHIRRLGEHAMLACSFQTLLSIQPALPPSTDAWFEQPGSTIDYRANATYAINLFFGLEGDKLKATALYDFNVVKKDKMQSMLVDFGNILQTMHKSPNSLIRDILAVPQQMRGPINSASL

SEQ ID NO: 255 - protein, 1309 aa, synthetic construct
MSSASFTAWKTYLEGYSPCSFPELVDAETSKPGLASVSTTLKDSHDRLQSFANEHSNNDVVAVFCAAWSLVLRAYTGQDTVCFGRATRNINACRIDFTSDTSILRILQTLQTACAEKRLTSDVPVSQLSLETGDLITSYFNTCVLSPNQERIQQGESDIIERGSTGLDLFGIVVIFEVKENVARVTVRYQTSLLQENQAISVINTLDQAITEIINHKDQIDDISLLSAVDKSQIYSWNAKLAHHPGYRVDELIGERCQLSPSALAVSAWDGELSYKGLDEASSSLAQHLTALGICPEVFVPVCFEKSRWAIVAILGVMKAGGAFVLLDPSHPHKRLLDICNKVSAKLIVCSTQHVKLAASLAPLIVELGDDDAKRNWHVEDFQKSDSLVTPGTALYAVFTSGSTGTPKGVVSEHTSFLAAMPTYIKSLGLNQESRVFQFASYAFDVTIFDTLMTLTVGGCVCVPSDTERSNDLAHAIQHFRATHMSLTPTVARILDPADFPTLQTLTLGGEKLVTSEFSKWVDHVRVVHLYGATECSMMSIQCITGTSSHLKTINHETGSACWVVDPENHDRLQAIGAIGELMVEGEIVGRGYLDDPVQTSETFIPPPGWLRKLRGNNCNQSVKIYKSGDLVRYAANGSLQFICRKNTQIKLRGQRIELGEVEHHLKLALPRARFVLAELITMPDSSRPPMLMAFVRPEDNSSSDSLTGGREAALTTIFAEPTDEFRSQVPATLSKLQNSLPSYMVPSAIIPLAVIPLTGTDKVNRKILRQLAAELPREQLERYQSTVRSYLAPTTNVEKALQKYFGEVLDLPLEQVWADDHFFSLGGDSLTAMKLVSMARRDNYRLTVKNVFNSPQLSDLAKLIKTTKNEDDEKLQPFALIDKKQDVIRAAAQQCHLPRRAIEDIYPCTSLQKGLISETMRDPSAFIAKLQLPLRSHIDISRLEQAWSDVAKANPILRTRMILSASYGLLQTVVRDNIPWTVSENAECEDLDVAIGKPLVQLVLCRPAPREQNPAKLVLMMHHSIYDGYTLPLIIMQLRTAYEGGVLDPRPVSPFIRYLASLPNCADYWIPVLKDLKAPVFPALPSKAYHPLPNSTAIYAAPVSSPSAKQYTPNTYVRLAWAITQSHHQKISDVFFGTVVSGRNAPVDNIDLMTIPTVATIPCRVTLDTDSVVGDMLHRIQNAATEGIPYEQFGLSNISRLGEYPAHACSFQTLLVMQPAADWDTDDFLQIPKSDANYRADATYAINLFCELEAERLEVTALYDENVVHEIKMQQILVDFGKALRSVRQTPTSVVGDVLNILDKSRKE

SEQ ID NO: 256 - protein, 1848 aa, synthetic construct
MSSKDGECTLKMVNKEGALEFWSSKLQVAEPCILTLSNQSTAVPTSSAIESMTIDLGGIGLPWSECVNTRDISSSAIFMTAWGMILRAYTDSDEVCFGHAFTARQGPMNGHPALYRPRLNILPCFLQFEGKATVLETVRKTRLSLDDAQSNQDVSLVEICNHMGVKNNASFNTCMLFSSDEQTSPNAEPDVLAQEVTLMEGCPQYDIIIHVSPLKQCVLLYQKHCLSNDQAVHIAGALRVALTSIINSPHLAIQKVEIFSPVDENKLLQWNKTAPIASESCLHDLINQQCDLHPDSLAVVAWDGSFTYKELDRLSNTLAQKLCLAEIGPDVFVAICADRCKWIPVAMLGIIKAGGAFCALDPAHPPSRLREMCKALGSSMVITTVEDAKKADQLALTTIVLGDDSMTESNGTKFSPGRAPPSKTQPSNALYSVFTSGSSGNPKGVVIEHRSFASTALASLKPLDIRPEDRILHFASYAFDLSVFEVLTALTAGASVAIPSEKARMECLPRAATELQATWTFLTPTVARMYQPSDFPTLRTISLGGELVQTSDIELWGSKNLITGYNPAECCPLGISGPANLSEANFIGWPFASHAAWVVDTRDFQKLVPVGAIGELIIEGPAVARCYLHDKNNALPDSPFITSPPQWLTRFRGKTSPDARLYRTGDLVRYGCNGAVHYIGRKDFQIKIRGQRVELGEIESQLRNAFSAGNHRVAVEAVNLSGCTTLVAFISVDSSDSETKRDAAPAVRLQDIKQELETYVEGVVTKLRGILPSSMIPSAFLPVTHIPVSKSGKVDHKQLRSLASSLSPEVLFRPKGASTSGTLPETDDERRLQALFALVLGISPSNIRADSDFFRLGGDSALAMKLLALVSQEERFDLTYSDIFQHPRLRDMLNASRSRSSFMNNDDESVSTDSSLPPFCLVKDANSLIDIASDQCGINKESIEDIYPCTSTQANLMASTVLDEDPCLSIRSFKLEGNIDKARLKQAWSIASKGHPILRTRIIQSNTGAFFQVVIKGDICWSDYTYSDDSNSQFRPSFGLGSPSVQLCFMKGRLLVAMHHALYDAWSLPRLLNEVDGAYCQLSVQRLLPLNCYVKHVAESLDSAASFWKAELEDADPTHFPVLPSGKTDRETIPTTTKSISTTMPIDSERKSNVEFELQLAWAITCYSYTKNEDVIFGVTSSGRNIALDGVDKMLGPIFAITPLRVPIDPDQEVTEAIGDLQYRSEEQAPYTHLGLHRISQLGQEAAAACQFQTLLVVEPSETEIKGTWFCEHEVIYDSIEPRNHSLTLKCLVNPGFVDITAIFHQAILPDTQKQDMLLRFQHIFGQIHTSGTPDSTILDIDTSSPQDLSGLQHSSLFVKNVGRDYTTTNKLHNRDIFLYNSTLKSQIHHSVHMVDNALEEAAQAQKSPVPTGKRSESQQFAQASEAVNLVAEIDRYDLVITSVQQEEISDSKRSPQAKNMSFNLEAGKPITVFLTGASGFIGTEVLRQLLENHSIRRVIALVRGRSTQEVRSRTIDSAKKALWWTDGHGEKLDVWPGDLSLPHLGLDAEHWSMLEDGMTVDAVIHNGASVHWLKNYTALEATNVRSTAELLRIAVKTPHVRFTYVSSGRYQDPIEEVEKQVAMQFAAGGIPYSQTKFVAESLVRRAAARIPSEHSNLAVASLGLVIGTPTEGVVNSDDYIWRLVAACIKAGVCNADELDKWFPVSDVARTASTIIDTVLEPVTTSAPVKQVTGGITWREIWNIVMEMGYTVESRPAVDWLRTIRQDIEVEQEKHPLWTLAHMIEGSIDNTEPTWATSWHGNEAASARLKGAVKGSIAFLGEIGFIPKSGLEVQEQVQKIQIGAFSRS

SEQ ID NO: 257 - protein, 521 aa, synthetic construct
MSSDFLQRILGILSSEDVAPALFASSISVLFLILSYQVLYPPSKYSAFPTINGTRWLALSNRRILAEFVTDAQGLMRNGLEKYDIFRIISSIGPMTILHPKYTDEIHNDRQLNFMAVLAKEMFPNYPGFDLFREGTDGSTVLQDAVKFGSSRCLGKSTQLLSDETSTLLQKLWGDEPEWHEVTAKSSVHDIIAHLSALLFYGPELCSHKEWLEVTDEYASVGFLAARQLRLWPPILRPIAQWFLPACRRLRYLASRTRGLIEPVIAARQKEKAICYSHGRQPPVYDDAIEWTERAAKGRPYDAAMSPLLFSINALHTTTDLLTQVILDLSTQPDLIVALRQEILSVKPQQNGWKNASLNQLLLMDSAIKESQRLKPTESILMRRYAMDDLTLADGNKIPKGTVLGIPIFGMRDPKIYVDPDMYDGYRFQKMRDKPGFENKCQLVSTSPWHLGFGHGIHACPGRFLAAVQVKIILCYIVAKYDFKLAGGAPPKVQSVGIELISDTEARLAVRRRQEMVIGLE

SEQ ID NO: 258 - protein, 458 aa, synthetic construct
MSSDAQRLMRKGLEKYDVFRMISSIGPMTILHPKYTDEIHNDRRLNFMAVLAKEMFPKYPGFDLFREGTDGSTVLQDAVKFGSSRCLGKSTQLLSDETSTLLQRVWSDEPEWHEVTAKSSVHDIIAHLSALLFYGPELSGHKEWLEVTDEYASVGFLAARELRLWPQILRPIAQWFLPSCRRLRYLASRTRSLIEPVIAARQKEKTICASHGREPPVYDDAIEWTERAAKGRSYDAAMSPLLFSINALHTTTDLLTQVILDLSTQPDLIAALRQEIISVKPQQNRWKNASLNQLLLMDSAIKESQRLKPTESILMRRYAMDDLTLADGTQISKGTVLGIPIFGMRDPKIYVDPDTYDGYRFQKMRGQPGFESKCQLVSTSPWHLGFGHGIHACPGRFLAAVQVKIILCHIVAKYDFKLAGGAHPKVQSVGIELISDSEARLAVRRRQEMAIGLEKDQS

SEQ ID NO: 259 - protein, 522 aa, synthetic construct
MSSDLLRKFSGPLVSTNMEPALFSSIAFCFILVILVNQVLYPSSKSSPFPTINGRGFLELNDRRILTDFVMNAQGLMQRGLSKYDIFQMLTSIGPMTILHPKYTNEIYKNKRLNFMAVLAKEMFPTYPGFDLFREGTDGSTVLQDAVKFGSTRCLSRSTQFMSEEAGILLKKAWGDEPEWHDVTAKSSVHDIIGHISALLFYGPELSNNKEWLEVTEEYTSTGFLAARELRLWPQVLQPFIHWFLPSCRRLRYLARRTRGLIQPVIAARQRENARRASEGQDLLTYDDAIEWTERAAKGRPYDAAMSPLLFSINALHTTTDLLTQVLLDLSTQPHLIEALRQEFITVIPQQNGWKNTSISQLVLMDSVIKESQRLKPTESILMRRYAMHDITLADGTQIPRGTVLGIPIFGMRDPEIYPDPDTYDGYRFMKMREQPGFKGVSQLVETSPMHLGFGHGIHACPGRFLAATQVKIVLSHMVMKYDFKLAGTLEPKIQSVGIELISDSEATLAVRRRKEQMILGL

SEQ ID NO: 260 - protein, 294 aa, synthetic construct
MAAPARELRLWPQILRPIAQWFLPSCRRLRYLASRTRSLIEPVIAARQKEKARCASHGREPPVYDDAIEWTERAAKGRSYDAAMSPLLFSINALHTTTDLLTQVILDLSTQPDLIAALRQEIISVRPQQNGWKNASLNQLLLMDSAIKESQRLKPTESILMRRYAMDDLTLADGTQIPKGTVLGIQSSIYVDPDTYDGYRFQKMRDQPGFESKCQLVSTSPWHLGFGHGIHACPGRFLAAVQVKIILYHIVAKYDFKLAGGAHPKVQSVGIELISDSEAKLAVRRRQDMAIGLE

SEQ ID NO: 261 - protein, 362 aa, synthetic construct
MSSSDEMDKFLAETWGDEQEWHKVTAKTVGRNIIGRLSALIFYGKELRDNEEWLDVTDEYAYMGFMAARELRLWPQALRPFVQWLLPCCRRLRALARKTRGLVRPVLTARRKEKAWCEAQGKDLPEYHDAIEFMERAAKGRPYDAAMGPVLFSVNALHTTTDLLTQVLLDLSDQPALINALRQEVLTVMPHRNGWKSSTLENLKLLDSVIKESLRLKPSESILMRRYAMRDVALSDGSQIRKGTVLGISTFQMRDPEIYTKPDTYNGYRFFEMRKIPELEKKCPLVATSPAHLGFGHGIHACPGRFLAAIQIKIVMCHIIMKYDIRPAGAAKAKVHVHGIDLISDPEAEILVRKREQNVAFV

SEQ ID NO: 262 - protein, 496 aa, synthetic construct
MAAKKQVTFKPFRLSPVDHSLPKVYIFKSLYFRGVDDTGSLSRLQDGIDRLISCLPFLSGEVVPCADIPDKVGVLQVQMPCPSLQEIPMLLVKSYPNHTWPAASTSERWRNTALLDQSYRPLPDFIPPSKPRPVLRFQANFLADGLMLCMGYNHSVFDGTGAGNILEMLADCCRANPNSILALPTNGDIESELRGLLSSPGVAVANASQEAYAINCAHTEVEPEPSSAMLYCWPFLLSSEKIECLQEACNSLLPHIVRLYSGTQSSLINQDTNWPHILSSNDVLTALLAVSIEKAREATGALGHMSRSLAMAVNLRERLKPMPRHYLGNLVTTVWVSHHRPAVKDLETMVLPVPACNRHEIDRDDLLWITHVAFRIRLGLNAINEEHIRGLIHYLHSQDDWEQIGIHFTDPIFISSWRHLKVYELDFGPTIGHAEHFEMDVGTTDGVCVVMPANTRAVGKTKKAPWDIRIVLNPEVLQALIASAIFGWAMVKDAST

SEQ ID NO: 263 - protein, 501 aa, synthetic construct
MAAKPRATFKPFRLSPVDHSLPKVYIFKILYFQAVDFTCCLSLLQDGVDRLVSYLPFLTGEVAPCTALKGKTGVQQIQMPGPSLKELPMLLVKYYPDRILPKYPSPSKNSAESKEGTSSPDQSYRPLPDFIPASQPRPVLRFQANFLADGLVLCMGYNHSVFDGTGAGTILEMLADCCRAGAASDISLITTSDIESELRGFLSSASVAVPKDSQAEYAINCAHTEVETQSFPTMLCNYPFLLSSDRIERLRDACNGLLPYVVETYSRAQTSPVNNDGKWPDFLSSNDVLTALLAISVKRAREAAPSPQPRSTSLAMAVNLRERLKSVPSHYLGNLVTTVWASHLEPPKRPEALVSTCSTCEQLEIEMDDLLWIVEVAFRVRLGLNSIDEEHACGLIHYLHSQDDWEKIGIHFTDPVFVSSWRHLKVYRLDFGPGIGRIQNFEMDVGTSDGNCIVMPADFKKIDGKNKRPQWEVRLILNQEAMDALINDSLFSWARIKGTFE

SEQ ID NO: 264 - protein, 349 aa, synthetic construct
MSSGYNHSVFDGTGAGNVLEILADCCRADPASIVSLPTTGGIESELRGLLSDPGVAVANPCQAAYAINCAHTEVEPESFPAMLCSWPFLLSSEKIKCLREACNSLLPYLVRTYRGSQSSMIYPDPNWPQILSSNDVLTALLAVSVEKAREATGAQEHMSSSLAMAVNLRERLKPMPRHYLGNLVTTVWASHHRPAEKDPETMVSPVPACNHPEIDRDDLLWIAHVAFRIRLGLNAVNEEHIRGLLHYLHSQDDWEQIGIHFTDPIFISSWRHLKVYELDFGPGIGHVENFEMDVGTTDGVCVVMPANKRAVGKTEKAPWDIRIVLNSDVMEALITDTLFGWPMVRDASK

SEQ ID NO: 265 - protein, 349 aa, synthetic construct
MSSGYNHSVFDGTGAGNILEMLADCCRADPASIISLPTTESIESELRELLSDPGVAVANHSQAAYAINCAHTEVEPESFPAMLCSWPFLLSSEKIECLREACNSLLPHLVRTYRDSQSSMIYPDPNWPQILSSNDVLTALLAVSVEKAREATGAQEHMSSSLAMAVNLRERLKPMPRHYLGNLVTTVWASHRRPAEKDPETMVSPVPVCNHPEIDRDDLLWIAHVAFRIRLGLNAINEEHIRGLLHYLHSQDDWEQIGIHFTDPIFISSWRHLKVYELDFGPGIGNVENFEMDIGTTDGVCVVMPANKRAVGKTKKAPWDIRIVLNSGVMEALITDTLFGWAMVRDASK

SEQ ID NO: 266 - protein, 319 aa, synthetic construct
MAAGYNHSVFDGTGAGAILEMLADCCRAGAASETSLTTAGDIESEPRGFLSSASVAVPKDSQAEYVINCAHTEVEADSFPAMLCNYPFLLPSEKIERLRDACNSLLPYLVDTYSRSQKSRVNDDAKWPGFLSSNDVLTALLAIEVERAREAAPSPQPKSTSLAMAVNLRKRLTLVPSHYLGNLVTTVWTPHLQPTKRWDTLLSTSPACEQLEIEMSDLLWVADVAFRVRLGLSSIDEEHACGLIHYLHSQDDWEQIGIHFTDPVFISSWRHLKVYEPDFSPGIGQIQDFGMEVGTSDGAMDALMNDPLFGWARVIDTFE

SEQ ID NO: 267 - protein, 438 aa, synthetic construct
MSSTKTDAQGRHPQETATHAATTDEEVQDQWRAPFEVLSRTLVFQHEDHRLWWERAASKLATYLRLAKYSVGSQYQHLLMFYSVYAPNLGPWPNDKRDNVHWVCGICPGGENLEISMNYQQGAKCTVRIAAETITPAAGTDKDPFNLTAEKKMIEDLKALQPNLNFTWFNHFQREVLVPEEVALNNDEIISKVPFKNQRLHGLDLSEGAFMLKSYFMPAIRSAITGVENTQIMFESIRKLNLKNANFISALSTLEDWMVPTNGRFMEYWDGISYDAVDACKARIKIYTGIRMKSIEHARDVWTLGGRLQGEDIEKGFDLVARLWRRLMDEEPSTCEMKYWMQWVWELRTDVPFPVPKLYFSVAAAEDHYVSDTVVEILDYLGWDDLVQTHRALMDEAWSLGQTTKSYLAFSYISVTFHSIKGPYITTYGNPSGPRPVF

SEQ ID NO: 268 - protein, 398 aa, synthetic construct
MAATKSDAQGRHPQETAIHAATKDEVVQDQWRAPFEVLSRTLVFQHEGHKLWWERAASKLATYLRLAKYSLVSQYQHLLMFYSVYAPNLGPWPNEKRDNVHWVCGICPGGENLELSINYQQGSKCTVRIAAETITPAAGTDKDPFNLTAEKKMIEDLRAIQPDLDFTWFDHFQREVLVPEEVVLSNDDVLSKVPFKNQRLHGLDLSEGAFMLKSYFMPAIRSAITGIETTQIMFESIRKLNLKNANIISALNTLEDWMVPTNGCFMEYWDAVSYDAVDADKARIKVYTGICMKSLEQARDVWTLGGRLQDEDIEKGFDLVARLWRRLMDEEPSTCEMKYWMQWVWELRTDVPIPVPKLYFSVAAAEDHYVSDTVVEILDYLGWDEHVQTHRALMDEAW

SEQ ID NO: 269 - protein, 398 aa, synthetic construct
MAATKSDAQGRHPQQTAIHAAAKDEVVQDQWRAPFEVLSRTLVFQLEDHKLWWELAASKLATYLRLAKYSVASQYQHLLMFYSVYAPNLGPWPNEKRDNVRWVCGICPGGENLELSMNYQQGSKCTVRIAAETITPAAGTDKDPFNLTAEKKMIEDLQAIQPNLDFTWFDHFQREVLVPEEVVLSNDDVISKVPFKNQRLHGLDLSEGAFMLKSYFIPAIRSAITGIETTQIMFESIRRLNLKNANIVSALNTLEDWMVPTNGRFMEYWDGVSYDAIDADKARIKVYTGICMKTLEQVRDVWTLGGRLQGGDIEKGFDLVARLWRRLMDEEPSTCEMKYWMQWVWELRTDVPIPVPKLYFSVAAAEDHYVSDAVIEILDYLGWDEHVQTHRALMDEAW

SEQ ID NO: 270 - protein, 426 aa, synthetic construct
MAATKDTTQVSHSQEELRAPFEVLSRTLSFEHEDHRLWWEQGGSKLATYLKLARYSMASQYQHLLMFYSVYAPNLGPYPNEKRDNVNWVCAICPGGENLELSMNFQQRSKCTVRMAAETLGPLTGTEKDPTNVAAENKLIDDLRALQPDLNFTWFGHFEREVVVPKEVALNHKQAINSVPYKNQRLHGLDLAEGAFTLKSYFLPSIRSAVTGVDTTQIMFDSIRKLALGNPNFNSALDMLQDWMIPTRGRFMGDWDVVSYDAIDAEEARIKVYTGIKMESLEQAWDIWTLGGRLQGDVVDKGFKLVKRLWGRLMDDEPSTCELKYCMQWVWELRTDVSIPVPKLYFTVAEAEDHYVSDAVVEILEYLGWDEHVQTHRALMDEAWSLNQTKESYFAFNYISVTYHSIKGPYITTYGNPSGPRPVFKE

SEQ ID NO: 271 - protein, 395 aa, synthetic construct
MAATKGDVQVFHSEEKLRDPFEVLSLTLSFQHEDHRLWWEQGGFIRRIYLRLAKYSVASQYQHLLTFYSVYASNLGPWPNEKSDNVNWVCGISPGGKNLELSMNYQQQSKCTVRIAAETIGPLTGTEKDPSNMIAEKKLIEDLRALQPDLNFNCHKKAIDTVPFKNQRLHGLDLAEGAFMLKSYFMPSIRSAVTGVETTQIMFDSIRKLGLENNNFNSALDMLQDWMIPTNGRSMEHWDGVSYDAIDAEKARIKVYTVINMQSLELACEIWTLGGRLQGDVVDKGFELPSTCELKYCMQWVWELRTDVPIPVPKLYFTVAEAEDHYVSDAVVEILEYLGWDEQVQTHRALMDEAWSLNQAKKSYFAFNYISVTYHNIKGPYITTHGNPSGPRPVF

SEQ ID NO: 272 - protein, 361 aa, synthetic construct
MAAFYSVYAPNLGPWPNEKRDNVNWICGISPRGENLELSINYQRGSKSTVRMAAETIGGLAGTEKDPSNVIAEKKLIEGLRALQPDLNFTWFDHFEREVVVPEEVALSHKKVIDTVPFKNQRFHGLDLIEGAFMLKSYFVPLIRSAITGVETTQIMFDSIRKLALKNPNFNSALDMLQDWMIPTNGRFMEHCDGVSYDAIDAEKARIKIYSGIKMESLEQAREIWTLGGRLRGDIVDKGFELVKRLWRRLIDEEPSTCELKHCMQWVWELRTDVSIPVPKVYFTVSEAEDHTVSDAVVEILEYLGWDEHVQTHRALMDEAWSLNQTKTSFFAFNYISVTYHNIKGPYITTYGNPSGPRPVF

SEQ ID NO: 273 - protein, 316 aa, synthetic construct
MAATSQHQEHTGTKRFSIQSDPVEIHRAIVEDGVAIIEGFLTPEQVQKLNKDVDAPLKADREQLKFKADKKDDPHFWLADFIPDHVARVHNLVDFSHCFRHEILNHELLHKICRLTFEESGDYWLGYGAVIENGPGTTEQKWHRDQPRYPLVKEGPDAPEGMLNFFTALTDFDAETGKTQYILGSNKRVELGEPDADHPIEYVGLKPGDTTIVSGKITHRGSDNRSDKMRRAMPIMIIPSILTPFDATCHLSRELVETMTPLAQKMICRRSVMIPAPGTVEVKTGIWCVNMREAGEQIGLKSNQRAKEDAEATDAV

SEQ ID NO: 274 - protein, 316 aa, synthetic construct
MAATSQHQEHARTKRFSIESDPAEIHRAIVEDGVAIIEGFLTPEQVQKLNKDVDAPLKADRDQLKLKSDKKDDPHFWLADFIPDHVARVHNLVDFSHCFRHEILNHELLHKICRLTFQESGDYWLGYGAVIENGPGTTEQKWHRDQPRYPLVKEGPDAPEGMLNFFTALTDFDAETGKTQYILGSNKWVELGEPDADHPIEYVGLKPGDTTIVSGKITHRGSDNFSDKMRRAMPIMIIPSILTPFDATCHLSRELVETMTPLAQKMIGRRSIMIPAPGTIEVKTGIWCVNMREAGEQIGLKSNQLVKENAETTDAM

SEQ ID NO: 275 - protein, 316 aa, synthetic construct
MAATSQHQEHAGTKRFSIQSDPAEIHRAIVEDGVAIIEGFLTPEQVQKLNKDVDAPLKADREQSRLKPDKTNVPNFWMADFVPDHVARVHNLVDFSHCFRHEILNHELLHKVCRLTFQESGDYWLGYAAVIENGPGTSEQKWHRDQPRYPLVKEGPDAPEGMLNFFTALTDFDSETGKTQYILGSNKRVELGEPDADHPIEYVGLKPGDTTIVSGKITHRGSDNRSEKMRRAMPIMIIPSILTPFDATCHLSRELVETMTPLAQKMIGRRSVMIPAPGTIEIKTGIWCVNMREAGEQIGLKSNQRVKEDAEATDAV

SEQ ID NO: 276 - protein, 319 aa, synthetic construct
MSSTSQHQEHARTKRFSIQSDPAEIHRAIVEDGVAIVEGFLTPEQVQKLNKDADAPLKADREQLKLKPDKKDDTHFWLADFIPDHVARVHNLVDFSYCFRHEILNHELLHKICRLTFQESGDYWLGYGAVIENGPGTTEQKWHRDQPRYPLVKEGPDAPEGMLNFFTALTDFDAETGKTQYILGSNKRVELGEPDADHPIEYVGLKAGDTTIVSGKITHRGSDNRSDKMRRAMPIMIIPSILTPFDATCHLSRELVETMTPLAQKMIGRRSMMIPAPGTIEVKTGIWCVNMREAGEQIGLKSNQLVKEDAEASQATDAA

SEQ ID NO: 277 - protein, 316 aa, synthetic construct
MSSTSQQQEHARTKRFSIESDPAEIHRAIVEDGVAIIEGFLTPEQVQKLNKDVDAPLKADRDQLKLKSDKKDDPHFWLADFIPDHVARVHNLVDFSHCFRHEILNHELLHKICRLTFQESGDYWLGYGAVIENGPGTIEQKWHRDQPRYPLVKEGPDAPEGMLNFFTALTDFDAETGKTQYILGSNKWVELGEPDADHPIEYVGLKPGDTTIVSGKITHRGSDNFSDKMRRAMPIMIIPSILTPFDATCHLSRELVETMTPLAQKMIGRRSIMIPAPGTIEVKTGIWCVNMREAGEQIGLKSNQLVKEDAETTDAV

SEQ ID NO: 278 - protein, 306 aa, synthetic construct
MSSESPPQQRTKRFSMDSDPVAIHQAIVEDGVAVIEGFLTPEQVEKLNRDVDARLLAYRNRARPEVTDKQKLWTADFVLEHVARVHNLVDFSHVFRHEILNHGLLHEICRRTFWESGDYWLGFGAVIENGPGTEQQKWHRDQPIYPLVKQGPDAPEGMLNFFTALTDLDDETGRTQYIWGSNKWEELGTPDALHPIENVALKAGDTTIVSGKVTHRGSANLSRKFRRAMPIMMVPSILTPFDATCHLSRELVETMTPLAQKMVGRRSVMIPPPGTTEGVRTGIWCLNMRELGEQLGLKSNQPEQIV

SEQ ID NO: 279 - protein, 310 aa, synthetic construct
MSSAALSHPQPQIQRFPVTSDGAAVYKALADDGVVIIEGFLSPEQVTKLNRDVDAPLISMREDGKISKPPKEGQSTYWMADIVPQYVQRVHNLVGFSKVFRHEILNHELMHELCRLAFEVSGDYWLGYGAVMENGPGTHEQSWHRDQPNYPLLRSGPGTAEGMLNFFTALTDFTPENGRTQYIWGSHKLVEIGEPDAEHPVVFSDLKAGDTAVLSGKIVHRGSLNATLDTYRRALALIIIPGVLTPFDATCQLPRRVVETMTPLAQRMVGRRSVRVSPPSAIGIWCLNMREVGEQMGLKSNQPDEEGEEE

SEQ ID NO: 280 - protein, 309 aa, synthetic construct
MSSTVQSKPQAQVQRFSLTSDAAAVHKALADDGVAIIKGFLSPEQVAKLNADVDPPLFALRQAEKPANPSAASVWMADLHPAHVQRVHNLVGFSKVFRHDILNHELMHDVCRLAFQESGDYWLGYGAAIENGPGTQEQIWHRDQPDYALLKAGPGTAEAMLNFFTALTDFTPETGMTQYIWGSHKRVELGEPDAEHPVVFTKLKAGDTAVLSGKIVHRGSANATPDVFRRALALMIIPAIMTPFDATCHLSRHMVETMTPLAQKMVGRRSVVIPPPHTVGAALGIWCLNMREVGEQMGLKSNQPDKEEE

SEQ ID NO: 281 - protein, 310 aa, synthetic construct
MSSTTSSHTQPNIQRFPATSDGAAVYKALADDGVVIIEGFLSQEQVEKLNADVDHPLVSLRESGKLEKTFKEGQSTFWMTELVPQYVQRVHNLVDFSRVFRHDVLNHELLHQLCTLAFEPSGDYWLGYSAVIENGPGTKEQFWHRDQPNYPLLRSGPGTAEGMLNFFTALTDFTPENGRTQYIWNSHKRVEIGEPDAEHPVLFSDLKAGDTAVLSGKMVHRGSANITSDFYRRALATMIIPGVLTPFDANCQIPRRVVETMTPLAQRMIGRRSVRVSPPSAVGIWCLNMKEVGEEMGLKSNQPDVEGENE

SEQ ID NO: 282 - protein, 543 aa, synthetic construct
MSSPSDQKTRCIVIGAGVSGILMAYKLQHYLNDFVEFQILEKNPDLGGTWYENKYPGCACDVPSHCYQYAFAPNPSWSKLYSSSEEIHDYLKKVVKHFNLERYIRYNTKVTSAKWTESTGTWSVGISDGTTIDCEILINAGGILHHPQLPDLKGLKSFAGKVVHTAAWDSGLDLGGKKVALIGSGSSAVQILPQLQPVCSSVRVYIRTPPWIAPPMVDTESESPNRDYTNDEQAFFRQDPAKYAQMRKEINSNLNGMFKALFKNSPEQQNFRQMLETRMRSLIRDESLQDHLVPKFEVGCRRVNPCEQFLYSIQEKNVTAVFNAIEEVKPHGIVSEGVEHPVDIIVAATGFNNSFRPQFPLLGRDGVNLQDIWADEPTSYFGTGVSGFPNYLTFLGPNTPISNGAFMGPLEGTSDYFIRLLQRAIRYGASSFDVKPEAQADFDSRVYKKTRDGKITALWPGSSLQYMQVLAEDRWEDYNWAYRGRRYDYWGNGFSWIEQPKSDKLGIQERKSKNEMVTIPNIDADLGFYITQQTPLPEADTED

SEQ ID NO: 283 - protein, 556 aa, synthetic construct
MSSDASQQKLRCVIIGAGVSGILMAYKLQKHLREFVDFQVLEKNPELGGTWYENRYPGCACDVPSHCYQYSFAPNPSWSKIYSPSEEIHAYLKKVASHFNLERYIRYRTEVVSANWDEVTSSWNVGINDGTIMACDVLINAGGILYDPQWPSIEGLGRFNGKLIHTAAWNKKLDLKAKRVALIGSGSSAVQILPQLQPICSSIRVYIRTPPWIAPPMIETDLESPNRDYTSDEKASFRQNPEKYAEMRNGINSQFNGMFRALFKTSPEQHYLRQMLEARMRSLIRNEELQEHLIPRFEVGCRRVNPCEHFLRSIQEKNVTAVFSAIEEVTQNGIVSEGVEHPVDTIVAATGFDNSFRPKFPLRGRYGVNLRDLWANDPISYFGTGVAGFPNYLTFLGPNTPIANGAFMGPLEGTSDYFIRLLQRAIRFGASSFDVKAEAQADFDSRIHKFMENMAWTGSCRSWYKSGRDGKITALWPGSSLHYMQVLAEDRWEDYNWVYKRPRYDYWCNGLSWIEDPESDRLGFQMQKSREAMTTIASTDADLGFYIKQETPLPEI

SEQ ID NO: 284 - protein, 557 aa, synthetic construct
MSSDTSQQKRRCVIIGAGVSGILMACKLETHLGEFVDFHVLEKNPELGGTWYENRYPGCACDVPSHSYQYSFAPNPSWSKIYSPSEEIQAYLKKVVSHFGLEKRIQYGVEVVSADWDEASSTWAVGLKHGTTMKCDVLINAGGILHKPRWPEIEGLDRFKGALVHTAAWDKTLDLAGKTVGLVGSGSSAIQVLPHLQPICSSIRMYIRTPAWIAPSTMEKKEDPGPSNRDYTSDEKASFRQDPESYAEMRSAIDAQYNGIFPALFKSSPEQRHLRQVLEARMRSLVRDEALRDRLVPRFEVGCGRVNPHEDFLRGIQEENVTPVFDAIEAVTQNGIVSGGVEHPVDAIVAATGFDNSFRPRFPLRGRHGVDLADLWAEDPVSYFGTAVAGFPNYLTFLGPNTHIANGAFMGPLESTSDYFIRLLQRAIRFGASTFDVRPEAQADFDRRIHSFMGNMAWTGSCRSWYKSRRSGKVTALWPGSSLHYMQVLAEDRWEDYDWTYRRPRYEYWSKGLSWIEDPEGDRLGVQIQRSRETMTTIAGTDAHLAFYIKQTAPLPE

SEQ ID NO: 285 - protein, 578 aa, synthetic construct
MSSTLSECRNRPIHANRLMRVICIGSGASGLYLAYRLQSCFTDYTLDVYEKNADVGGTWFENRYPGCACDIPAHNYTYTFEPNANWSANYASSSEIARYFSGFADKYGLRRYIKFEHEVIGARWDEARSEWSVRVRKTDGKVFEHACDFLINAAGILNAWKLPDITGLDSFKGTLVHTARWNEAIDVAGKTVGLVGNGSSGIQILPELQRTAQHVTAFLRGPTWVSPARGMEYHIYTEEERKRFQDTPTELLAMRKQTESFMTSATLFSLFIKGSKAQEVLAQDMEDHMRMKIDNDLLAQALIPEYPLGCRRPTPGINYLESLAKPNVRAYHGTIKEVTASGCITDDGSVHPLDILVCATGFDSSFCPRFPIVGRNGVNLAAQWAEEPRNYLSLAVHNFPNYFMFLGPNCPIGSGPIMVCIEAACEYMANFMNRWQKEDIKFFDPSEEAINDFMEQKDLFMDMTVWKSGCRSWWRNNETGKVTALWPGSTLHYLEALAYPRYEDFEVQYASRNRFAYLGNGFSQRQLDPSADQTYYLRQRDEGSVLSNGMSTRNVKDLGDLLTQCRFGPGGSEGNSTE

SEQ ID NO: 286 - protein, 574 aa, synthetic construct
MAATIQKCRDRPIHANRTMRVICVGAGVSGLYLAYKLRKHFTDYILDIYEKNADIGGTWFENRYPGCACDIPAHNYTYTFEPNPNWSASYASAAEIFEYLSGFAEKYDLRQFITCHHEVVGALWKEDQAAWSVKVRKPNGAIFEQQCDFLINGAGILNAWRWPDIAGLDSFQGTLVHTAQWDAAIDVEGKKVGIIGNGSSGVQILPEIQRRAQHVTAFSRGPTWITPSPAMEHHVYTDEEKQLFRDNPSKLLNMRKSSEAIMASASLFSTFLKGSNAQDVFRSQMEIHMKGKIHDDNLAKKLVPDFPFGCRRPTPGTDYLESLTKPNVTICHEAIEEITPSGCVTADGQEHLVHILVCATGFDNTFCPRFPIIGRDGANLAEEWEKEPRNYLSVAVNGFPNYFMFTGPNCPFGSGGIIVGIEAEGTYMTNFFNRWQKEDIKFFDPKKEAVDEFMEQKDLFMDTTVWAKGCRSWWKHKKTGKVTALWPGSTLHYLEALAHPRYDDFEVQYACSNRFSYLGNGFSQRQLDPTADLVYYLRESDEGSVFADGMSTRNAKDLGGSAIWGIAKGRKINP

SEQ ID NO: 287 - protein, 570 aa, synthetic construct
MAATIQKCRDRPIHANRTMRVICVGAGVSGLYLAYKLRKQFTDYILDIYEKNPDIGGTWFENRYPGCACDIPAHNYTYTFEPNPNWSTNYASAVEIFEYFSGFAKKYDLRQFITCHHEVVGAFWKEDRAEWSVKVRKPNGAIFEQRCDFLINGAGILNAWRWPDIAGLDSFQGTLVHTAQWDEAINMEGKKVGVIGNGSSGVQILPRIQRRAQHVTAFSRGPTWITPSAGMEHHVYTDEEKQRFRDNPSELLDMRKSSEAVMASAGLFSTFLRGSHAQDVFRSQMENHMRGKILDDNLAKKLVPDFPFGCRRPTQPGTDYLESLTRPNVTICHEAIEEITPSGCVTADGQEHLVDILVCATGFDNTFRPRFPIIGRDGANLGEEWEEEPRNYLSVAVNGFPNYFMFTGPNCPFGSGGIIVGIEAEGTYMTNFFNRWQKEDIKFFDPKKQAVDEFMEQKDLFMDTTVWAKGCRSWWKHNKTGKVTALWPGSTLHYLEALAHPRYDDFEVQYACSNRFSYLGNGFSQRQLDPTADLVYYLRERDEGSIFADGMSTRNAKDLGGILTEKLAMP

SEQ ID NO: 288 - protein, 576 aa, synthetic construct
MSSHSRPIHANRQLRVICIGAGASGLYLAYRLKKSFTDFTLQIYEKNADVGGTWFENRYPGCACDIPAHNYTYTFEPNPAWSANYATASEIAAYFAGFADKYGLREYIACQHEVVHAEWDEDRAEWMVRVRRSADGTVLEQRCDFLINAAGILNAWKYPDIPGLDTFEGTLVHTAHWDETLDVGGKRVGLIGNGSSGVQVLPELQRKAQHVTAFIRSATWVSPARGMEYHQYTEEEKRNFRENPDQLLAVRKQTEGFMTSAALFSMFIQGSKAQEVLAQDMETQMRAKLGSDHVLAKSIIPDFPLGCRRPTPGINYLESLGQPNVTACPGPIKEVTRTGCIAADGSAHELDVLVCATGFDTTFRPRFPVLGREARDLATQWADEPRNYLSLAARHFPNYFMFVGPNCPIGSGPIMVCIEAAGDYMARMLNRWQKEGIRSFEPKAAAVDDFMTYKDRFMASTVWHARAGSGRCRSWWRDSRSGQVTALWPGSTVHYLEALATPRYDDYDVDYVGGNRFDYLGNGFAQRQLAANADPVWYLRTADEGELLPDLMSTENAKDLGSLLRDGFEWTPAQGA

SEQ ID NO: 289 - protein, 521 aa, synthetic construct
MAANVSQQKLRCVIIGAGVSGILMAYKLQKHLREFIDFQVLEKNPELGGTWYENRYPGCECDVPSHCYQYSFAPNPSWSKIYSPAEEIHAYLKGVASHFNLERYIRYRTEVVSANWDEATSTWNVGISDGSTIACDVLINAGGILHDPHWHDIEGLGSFSGKLIHTAAWAKDLDLKGKRVALIGSGSSAVQILPQLQPICSFIRVYIRTPPWIAPPIIETDLESPNREYTSDEKASFRQNPEKYSQLRNGLNSQINGMFRALFKTSPEQHYLRQMLEARMRSLIRSEELQEHLIPRFEVGCRRVNPCEQFLRSIQEENVTAVFSAIEEVTQNGIVSEGVEHPVDTIVAATGFNNSFRPKFPLRGRYEVNLQDLWANDPISYFGTGVAGFPNYLTFLGPNTPIANGAFMGPLEGTSDYFIRLLQRAIRFGALSFDVKAEAQADFDSRIHKFMENMAWTGSCRSWYKSGRDGKITALWPGSSLHYMQVLAEDRWEDYNWVYKRPRYDYWCNGLSWIEDPETAG

SEQ ID NO: 290 - protein, 337 aa, synthetic construct
MAADKIVPQTDEIFNDFKIIDEPFVTRDNHPILAHILLPKEAPPKPSPVIINWHGGYLVAGHGLFALFFPPLLLQLAKKHSAIIVSPDHTLLPHKDGLVAVQEDAEAFHEWLHSSFPKVLAERTPGFQVDLSRILLHGGSSGAYLALSSALAHPDSYRSLSLMYPMIDFDTDWWRRGSRAVGAPNPGCLPDAAFPANDNTVKDQLEKFMKEPIVTMPGAERDGFGAWVARAGLFPELFNPGGKLDDDAAVWLNRRVKQGAKLPERIWVLHADEDTVVPIETSLTFQETMKQQQRPVRLDIVKGLDHCCDMAITEDWKGSQDPVILEAADWLVEKWLE

SEQ ID NO: 291 - protein, 340 aa, synthetic construct
MAADNIVLQTSEIFDDFTIIDQPFWTRENHPILAHIILPRSYLSSSSKPSPVIINWHGGYLVAGHGLLPLFFPPLLLSLAKKHSAIIISPDHALLPRKEGLSSVQADAEAFHHWLHSSFADTLAEKAPSYEPDLTRILLHGGSSGAYLALSSALSHPKSYRCLSLMYPMIDFDTDWWRKGSRAVGAPNPGCLPDAAFPVDDQVVMDQLEKFREGPIVTIPGAERDGFGACVARAGLFPEIFNPGGKLDDDSAVWLNRRVEQGSELPERIWVLHADGDTVVPVETSLSFAQTMKQQDRPVRLDIVKGLDHCCDMAVTEDWKGSDDPVVFGAAEWLVEKWLE

SEQ ID NO: 292 - protein, 340 aa, synthetic construct
MAADKVVLQTNEIFDDFTVFDQPFVTREGHPVLAHVLLPRCLPGSSSQPSPIIVNWHGGYLVGGHGVLAMFFPPLLLRLAKKHSAIIVSPDYALLPRKEGLPSVQADAEAFHGWLHSSLAETLAEKAPGREADLTRVLLHGGSAGAYLALSSALAHPRSYRCLSLMYPMIDFDTDWWRRGSRAVGAPNPARLPDAAFPADDQAVVDQLEKFREGPTVTMPGDERDGFGAWLARAGLFSEILNPGGGLDDDSAAWLNRRVKQGAELPGRIWVLHADGDTVVPIETSLGFAQTMKLQGRPVRLDIVRGLDHMCDMAVTEDWKGSDDAVVLDAAEWLVEKWLE

SEQ ID NO: 293 - protein, 343 aa, synthetic construct
MAASPPEMPTGYESKTITFKNIGALSLELDIMYPRGKNPEPVPVLIHYHGGFLVVGDKYSFMPYWLLNACASRKWIFVTPDYRLIPEATAHEAVADALDAYNWVRQSLSQALGMKIGPVISAGSSAGAYLALTSASMTSIKPDALVLIYGMLDPISERYTKPGTNIFGMPPIDSQPLLDQWNLLKEKGETTVHTGYPVPRDPANDFRMGLITALHCMALFPDFMTGVPGLGRDIAQNGTSVVPAEARQLYTIAFERGTGLPRTAIFHGKNDLAVPFSGSEAAADSLRKLGVVVHTEFPDDAPHSFDSMAGNVDLEDSDDTSTKTPAFDGLRGIVKFLDSVVAF

SEQ ID NO: 294 - protein, 344 aa, synthetic construct
MSSTQSIELPAGYLTKTTTYKIIHSVNLELDIVYPQETSVDPVPVLIHYHGGFLIIGDRFSFLPYWLLNACAARKWIFVTPNYRLIPESTAHDAVADALDAFLWVKTSLSRTLGVTVGPVISAGSSAGAYLAMISASVSSVKPDALVLLYGMLDPSSERYTTPGTNILGMPPIDGQPILDQFETLKQQGRANPLAGYPVPSDLASDFRMGLIAAAHCMGVLPDLMTSIPGLSRDIARHSTVAIPTEAKHLFPIGCSGDVVLPRTAILHGKNDNVVPFAASQVAADTLRKMGTEVYTEFLDDACHSFDTMVGNINIEKSNAPEARTPAFDGLRRIIKFLDEIARP

SEQ ID NO: 295 - protein, 344 aa, synthetic construct
MSSTQSIELPTGYLTKTVTYKIIHSVDLELDIVYPQETSADPVPVLIHYHGGFLIIGDRLSFLPYWLLNACAARKWIFVTPNYRLIPEATAHDAVADALDAFLWVKTSLSRTLGVTVGPVISAGSSAGAYLAMISASVSSVKPDTLVLLYGMLDPSSEWYTTPGTNIFGMPPVDGQRILDQFETLKQQGRAKPLAGYPVPSDPACDLRMSLIAAAHCMGVLPDLMTSIPGLSRDIACHSTVAIPAEAKYLFPIGCSGDIALPRTAILHGKNDNAVPFAASQVAADTLRKLGTKVYTEFLDDASHSFDTMFGNIDIEKSDAPETKTPAFDGLRRIIKFLDEIARP

SEQ ID NO: 296 - protein, 351 aa, synthetic construct
MSSAPPEMPSGYEMKTLPYKTTGSLSLNLDIIYPLQASTTPVPVLIHYHGGFLIVGDRQSFLPHWLVNACAARGWIFVTPDYRLLPETTAHDAVADAVDAYQWVRTSLASTLQHLSPGPVIAAGASAGAYLALTSASQAEHKPDALLLLYGMLDPLHEHFTHPVDNFFGMTPSDSRPFFDAWAAGQQKDDPAATVRTGYPIPADPSTDARMGLIAALHGQALLPDYMAGVPGLAGRVRDGSGGEVVDLVPEKARGLFAIACGRVAGLPPTAVVHGRNDVGVPVTISLSATETLRRLGVQVHAEFPDDAPHGFDAMLGDVDIERSRKAEGDTPALESLRRVLGWLDALVGRV

SEQ ID NO: 297 - protein, 340 aa, synthetic construct
MAADNIILQTSQIFNDFTIIDQPFLIRENHQVLAHILLPRNYSGSPSKPSPIIINWHGGYLVAGHGLLPLFFPPLLLRLAKKHSAIIVSPDHALLPREEGLSSVQADAEAFHHWLHSSFAELLAATAPSYEPDLTRILLHGGSSGAYLALSSALSHPKSYRCVSLMYPMIYFDTDWWRKGSRAVGAPNPGRLPDTAFPVDDQVVMHQLEEFRQGPVVTMPGAERNVFGACVARAGLFLEIFNPGGKLDDDSAVWLNRRVEQGSELPEHIWVLHADCDTVVPIETSLRFAQTMKRQGRPIRLDIVKELDHCCDMAVTEDWKGSDDPVVLDAAEWLVEKWLE

SEQ ID NO: 298 - protein, 524 aa, synthetic construct
MAAQLPSSTKILVVGGGPAGSTAATLLAREGFEVTLVEKAIFPRYHIGESLLISVQPIIDLLGAREAVEAHGFQRKKGVLWEWGGERWLFDWKKLRYDYTFHVKREEFDEILLRNAQKNGVKVFEGIDISRLEFDGERPVAAKWSKSSTGESGTIQFEFLLDASGRAGLMATQYLRSRMFMKAFQNVATWGYWKGATIPEVEVEGPITVGSIPYGWIWGIPLRDQTMSVGLVIHQELFKEKRATQSVEEIYHEGLKASPLFQDVVLKGATLEPQIRTETDYSYISRTLAGPGFFLVGDSGAFIDPLLSSGVHLAMHSALLAAASVKSIIAGEVDMASATEFYQRCYQGHFLRWALIVASFYEVNARKETYFWTAQQLAHEELGVFNMSQADMKDVFATMVSGVVDLGDAQNAGRLQKGAERVHQYLDDDGREEDVTALLQKSKQRIFEYLDRVKNRDSRAAMQRYKAGGTETFSMGLDADGAVGGLYVTTEPRLGLLRKVVEERAEAATEAPAPAAPPPAVAEV

SEQ ID NO: 299 - protein, 533 aa, synthetic construct
MAADNRIKTVVILGGGTAGWMTAAYLGKALQNTVKIVVLEAPTIPRIGVGEATVPNLQRAFFDYLGIPEEEWMRECNASYKMAVKFINWRTPGEGSPDPRTLDDGHTDTFHHPFGLLPSADQIPLSHYWAAKRLQGETDENFDEACFADTAIMNAKKAPRFLDMRRATNYAWHFDASKVAAFLRNFAVTKQAVEHVEDEMTEVLTDERGFITALRTKSGRILQGDLFVDCSGFRGLLINKAMEEPFIDMSDHLLCNSAVATAVPHDDEKNGVEPYTSSIAMEAGWTWKIPMLGRFGSGHVYSDHFATQDEATLAFSKLWGLDPDNTEFNHVRFRVGRNRRAWVRNCVSVGLASCFVEPLESSGIYFIYAAIHMLAKHFPDKTFDKVLVDRFNREIEEMFDDTRDFLQAHYYFSPRVDTPFWRANKELKLADSIKDKVETYRAGLPVNLPVTDEGTYYGNFEAEFRNFWTNGSYYCIFAGLGLMPRNPLPALAYKPQSIAEAELLFADVKRKGDTLVESLPSTYDLLRQLHGAS

SEQ ID NO: 300 - protein, 525 aa, synthetic construct
MAANTRNPDKVVIVGGGTAGWMTASYLKKAFGERVSVTLVESGTIGTVGVGEATFSDIRHFFEFLDLREEEWMPACNATYKLAVRFQDWQRPGHHFYHPFEQMRSVDGFPLTDWWLQNGPTDRFDRDCFVMASLCDAGRSPRYLNGSLLQQEFDERAEEPAGLTMSEHQGKTQFPYAYHFEAALLAEFLSGYSKDRGVKHVVDEVLEVKLDDRGWISHVVTKEHGDIGGDLFVDCTGFRGVLLNQALGVPFVSYQDTLPNDSAVALQVPLDMEARGIPPYTRATAKEAGWIWTIPLIGRIGTGYVYAKDYCSPEEAERTLREFVGPEAADVEANHIRMRIGRSEQSWKNNCVAIGLSSGFVEPLESTGIFFIHHAIEQLVKHFPAGDWHPQLRAGYNSAVANVMDGVREFLVLHYLGAARNDTRYWKDTKTRAVPDALAERIERWKVQLPDSENVFPYYHGLPPYSYMAILLGTGAIGLRPSPALALADPAAAEKEFTAIRDRARFLVDTLPSQYEYFAAMGQRV

SEQ ID NO: 301 - protein, 519 aa, synthetic construct
MAAINSVLIVGGGTAGWMTAAYLSKAFDKNINITVVESKEVKKIGVGEATFSTVRHYFDYLGLNESEWLPECSGSYKLGIRFENWDGQGNHFYHPFERWEVVKGFPISEWWLSKKLKDQRFDYDTFLTPHLCEAKRSPRRLDGSLFAQSIDKSLGQSTLAEQRAQYPYAYHFDADGVASFLKRYAMNRGVKHIEDDVTHVEIDTNGNIGYLEAKIYGKLRADLYIDCTGFKGLLINKALNEPFISFSDVLKNNRAVALRVPRENENDIEPYTTARTMSNGWRWTIPLYKRNGYGYVYCNKYQSPEEAEMELRKSIPYENAECVANHIRMRIGRSNRSWVKNCVAIGLSSAFVEPLESTGIFFIQHGIEQLVRFFPRSSGNELLIEEYNTRVNRVVDGVKEFLLLHFSLAQRNDTLYWKEWKNVELPKELLKKITLAQDHLLDKETIYPFYHGFEEYSWNTMILGLCSGSLNNKPALSLMTSDEADEMLLQHYMKAENMVNNLPTCYEYLKHIHDLKNKN

SEQ ID NO: 302 - protein, 532 aa, synthetic construct
MAAACTGWTSPLPLCVCLLLTCGFAEAGKLLVVPMDGSHWFTMRSVVEKLILRGHEVVVVMPEVSWQLGRSLNCTVKTYSTSYTLEDLDREFKAFAHAQWKAQVRSIYSLLMGSYNDIFDLFFSNCRSLFKDKKLVEYLKESSFDAVFLDPFDNCGLIVAKYFSLPSVVFARGILCHYLEEGAQCPAPLSYVPRILLGFSDAMTFKERVRNHIMHLEEHLLCHRFFKNALEIASEILQTPVTEYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 303 - protein, 530 aa, synthetic construct
MAAAPVAFPTSFFLCLLLASGLAQAGRLLVVPMDGSHWFTMQMVVEKLIHRGHEVVVVIPEVSWQLGKSLNCTVKTYSISHTLEDLDREFKYLSYTQWKTPEHSIRSFLTGSARGFFELTFSHCRSLFNDKKLVEYLKQRFFDAVFLDPFDVCGLIVAKYFSLPSVIFARGVFCDYLEEGAQCPSLPSYVPRLFSKYTDTMTFKERVWNHLIYIEEHAFCSYFLRTAVEVASEILQTPVTMTDLFSPVSIWLLRTDFVLEFPRPVMPNMVFIGGINCLQKKSLSKEFEAYVNASGEHGIVVFSLGSMVSEIPEKKAMEIAEALGRIPQTVLWRYTGTRPSNLAKNTILVKWLPQNDLLGHPKTRAFITHSGSHGIYEGICNGVPMVMMPLFGDQMDNAKRMETRGAGVTLNVLEMTADDLENALKTVINNKSYKENIMRLSSLHKDRPIEPLDLAVFWVEYVMRHKGAPHLRPAAHDLTWYQYHSLDVIGFLLAIVLTVVFIVFKCCAYGCRKCFGGKGRVKKSHKSKTH

SEQ ID NO: 304 - protein, 532 aa, synthetic construct
MAAARAGWTSPVPLCVCLLLTCGFAEAGKLLVVPMDGSHWFTMQSVVEKLILRGHEVVVVMPEVSWQLERSLNCTVKTYSTSYTLEDQNREFMVFAHAQWKAQAQSIFSLLMSSSSGFLDLFFSHCRSLFNDRKLVEYLKESSFDAVFLDPFDTCGLIVAKYFSLPSVVFTRGIFCHHLEEGAQCPAPLSYVPNDLLGFSDAMTFKERVWNHIVHLEDHLFCQYLFRNALEIASEILQTPVTAYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 305 - protein, 533 aa, synthetic construct
MAAAPADFPASLPLCVCLLLASGLAQAGRLLVVPMDGSHWFTMQTVVEKLLHKGHEVVVVVPEVSWQLTKPLNFVVKTYAVSHTQEDLNREFKIFIDAQWKSQQEGGILPLLDSPAKGFFELLFSHCRSLFNDKKLVEYLKQTSFDAVFLDPFDVCGLTVAKYFSLPSVVFSRGIFCHYLEDAAQCPSPPSYIPRMLLKFTDTMTFKERTRNLLAYMGERAFCHKFFKSAADIASEVLQTPVTMTDLFSPVSIWLLRTDFVLEFPRPVMPNVIYIGGINCHQGKPLSKEFEAYVNASGEHGIVVFSLGSMVSEIPEKKAMEIAEALGRIPQTVLWRYTGTRPSNLAKNTILVKWLPQNDLLGHPKTRAFITHSGSHGIYEGICNGVPMVMMPLFGDQMDNAKRMETRGAGVTLNVLEMTADDLENALKTVINNKSYKENIMRLSSLHKDRPIEPLDLAVFWVEYVMRHKGAPHLRPAAHDLTWYQYHSLDVIGFLLAIVLTVVFIVFKCCAYGCRKCFGGKGRVKKSHKSKTH

SEQ ID NO: 306 - protein, 688 aa, synthetic construct
MTTLLNPYFGEFGGMYVPQILMPALRQLEEAFVSAQKDPEFQAQFNDLLKNYAGRPTALTKCQNITAGTNTTLYLKREDLLHGGAHKTNQVLGQALLAKRMGKTEIIAETGAGQHGVASALASALLGLKCRIYMGAKDVERQSPNVFRMRLMGAEVIPVHSGSATLKDACNEALRDWSGSYETAHYMLGTAAGPHPYPTIVREFQRMIGEETKAQILEREGRLPDAVIACVGGGSNAIGMFADFINETNVGLIGVEPGGHGIETGEHGAPLKHGRVGIYFGMKAPMMQTEDGQIEESYSISAGLDFPSVGPQHAYLNSTGRADYVSITDDEALEAFKTLCLHEGIIPALESSHALAHALKMMRENPDKEQLLVVNLSGRGDKDIFTVHDILKARGEIGGGSGEGRGSLLTCGDVEENPGPMERYESLFAQLKERKEGAFVPFVTLGDPGIEQSLKIIDTLIEAGADALELGIPFSDPLADGPTIQNATLRAFAAGVTPAQCFEMLALIRQKHPTIPIGLLMYANLVFNKGIDEFYAQCEKVGVDSVLVADVPVEESAPFRQAALRHNVAPIFICPPNADDDLLRQIASYGRGYTYLLSRAGVTGAENRAALPLNHLVAKLKEYNAAPPLQGFGISAPDQVKAAIDAGAAGAISGSAIVKIIEQHINEPEKMLAALKVFVQPMKAATRS

SEQ ID NO: 307 - protein, 424 aa, synthetic construct
MGGASASGVDNKFNKERVIAIGEIMRLPNLNSLQVVAFINSLRDDPSQSANLLAEAKKLNDAQAPKGGSSASSAGGSSVDNKFNKEAQTAGVEIMELPNLNTRQLLAFIQSLRDDPSQSANLLAEAKKLNDAQAPKTSGGSSASSAGGSSVDNKFNKERVIAIGEIMRLPNLNSLQVVAFINSLRDDPSQSANLLAEAKKLNDAQAPKGGSSASSAGGSSVDNKFNKEAQTAGVEIMELPNLNTRQLLAFIQSLRDDPSQSANLLAEAKKLNDAQAPKTSGSGASGSGAGGSSVDNKFNKERVIAIGEIMRLPNLNSLQVVAFINSLRDDPSQSANLLAEAKKLNDAQAPKGSGASGSGAGGSSVDNKFNKEAQTAGVEIMELPNLNTRQLLAFIQSLRDDPSQSANLLAEAKKLNDAQAPKTS

SEQ ID NO: 308 - protein, 70 aa, synthetic construct
MVDNKFNKELGWATWEIFNLPNLNGVQVKAFIDSLRDDPSQSANLLAEAKKLNDAQAPKGSSSSGSSSSG

SEQ ID NO: 309 - protein, 70 aa, synthetic construct
MVDNKFNKETIQASQEIRLLPNLNGRQKLAFIHSLLDDPSQSANLLAEAKKLNDAQAPKGSSSSGSSSSG

SEQ ID NO: 310 - protein, 56 aa, synthetic construct
MQLLRCFSIFSVIASVLAQELTTICEQIPSPTLESTPYSLSTTTILANGKLDKRGS

SEQ ID NO: 311 - protein, 72 aa, synthetic construct
MKVLDLLTVLSASSLLSTFAAAESTATADSTTAASSTASCNPLKTTGCTPDTALATSFSEDFSSSSLDKRGS

SEQ ID NO: 312 - protein, 51 aa, synthetic construct
MLSLKTLLCTLLTVSSVLATPVPARDPSSIQFVHEENKKRYYDYDHGSLGE

SEQ ID NO: 313 - protein, 33 aa, synthetic construct
MKFAYSLLLPLAGVSASVINYKRDGDSLDKRGS

SEQ ID NO: 314 - protein, 66 aa, synthetic construct
MKLQSLLVSAAVLTSLTENVNAWSPNNSYVPANVTCDDDINLVREASGLSDNETEWLKKRDAYTKE

SEQ ID NO: 315 - protein, 20 aa, synthetic construct
MLLQAFLFLLAGFAAKISAS

SEQ ID NO: 316 - protein, 99 aa, synthetic construct
MKLSATTLTAASLIGYSTIVSALPYAADIDTGCTTTAHGSHQHKRAVAVTYVYETVTVDKNGQTVTPTSTEASSTVASTTTLISESSVTKSSSLDKRGS

SEQ ID NO: 317 - protein, 92 aa, synthetic construct
MRFPSIFTDVLFAASSALATPVNTTTEDETAQIPAEAVIGYSDLEGDFDVAVLPFSNSTNNGLLFINTTIASIAAKEEGVSLEKREATNFGS

SEQ ID NO: 318 - protein, 100 aa, synthetic construct
MEEQREILEQLKKTLQMLTVEPSKNNQIANEEKEKKENENSWCILEHNYEDIAQEFIDFIYKNPTTYHVVSFFAELLDKHNFKYLSEKSNWQDSIGEDGG

SEQ ID NO: 319 - protein, 75 aa, synthetic construct
MFRIQLRTMSSKTCKSDYPKEFVSFLNSSHSPYHTVHNIKKHLVSNGFKELSERDSWAGHVAQKGKYFVTRNGSS

SEQ ID NO: 320 - protein, 297 aa, synthetic construct
MSDSSPTINFINFNQTGTCISLGTSKGFKIFNCEPFGKFYSEDSGGYAIVEMLFSTSLLALVGIGDQPALSPRRLRIINTKKHSIICEVTFPTSILSVKMNKSRLVVLLQEQIYIYDINTMRLLHTIETNPNPRGLMAMSPSVANSYLVYPSPPKVINSEIKAHATTNNITLSVGGNTETSFKRDQQDAGHSDISDLDQYSSFTKRDDADPTSSNGGNSSIIKNGDVIVFNLETLQPTMVIEAHKGEIAAMAISFDGTLMATASDKGTIIRVFDIETGDKIYQFRRGTYATRIYSIS

SEQ ID NO: 321 - protein, 57 aa, synthetic construct
MIALPVEKAPRKSLWQRHRAFISGIVALIIIGTFFLTSGLHPAPPHEAKRPHHGKGP

SEQ ID NO: 322 - protein, 35 aa, synthetic construct
MFSLKALLPLALLLVSANQVAAKVHKAKIYKHELS

SEQ ID NO: 323 - protein, 99 aa, synthetic construct
MKLENTLFTLGALGSISAALVIPNLENAADHHELINKEDHHERPRKVEFTKDDDEEPSDSEDKEHGKFHKKGRKGQDKESPEFNGKRASGSHGSAHEGG

SEQ ID NO: 324 - protein, 164 aa, synthetic construct
MKAFTSLLCGLGLSTTLAKAISLQRPLGLDKDVLLQAAEKFGLDLDLDHLLKELDSNVLDAWAQIEHLYPNQVMSLETSTKPKFPEAIKTKKDWDFVVKNDAIENYQLRVNKIKDPKILGIDPNVTQYTGYLDVEDEDKHFFFWTFESRNDPAKDPVILWLNGG

SEQ ID NO: 325 - protein, 75 aa, synthetic construct
MTKNFIVTLKKNTPDVEAKKFLDSVHHAGGSIVHKFDIIKGYTIKVPDVLHLNKLKEKHNDVIENVEEDKEVHTN

SEQ ID NO: 326 - protein, 78 aa, synthetic construct
MKSFITRNKTAILATVAATGTAIGAYYYYNQLQQQQQRGKKNTINKDEKKDTKDSQKETEGAKKSTAPSNPPIYPVSS

SEQ ID NO: 327 - protein, 117 aa, synthetic construct
MAMFCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKEGSGSG

SEQ ID NO: 328 - protein, 171 aa, synthetic construct
MADRDRSGIYGGTYGQHQQGGGRPMGEQVKGMIQDKGPTASQALTVATLFPLGGLLLVLSGLALAASVVGLALATPVFLLFSPVLVPAALLIGTAVTGFLTSGALGLGGLSSLTCLANTARQAFQRTPDYVEEARRRMAEAAAHAGQKTAQAGHAIQSRAQEAASGGAGAG

SEQ ID NO: 329 - protein, 93 aa, synthetic construct
MSTLQRRRVNRADSGDTSSIHSSANNTKGDKIANIAVDGDDDNGTNKKIAYDPEESKLRDNINIPTLTLMEEVLLMGLRDREGYLSFWNDSIS

SEQ ID NO: 330 - protein, 88 aa, synthetic construct
MSLPLRTIVHLVKPFACTARFSARYPIHVIVVAVLLSAAAYLSVTQSYLNEWKLDSNQYSTYLNIKPDELFEKCTHYYRSGGNAAIRS

SEQ ID NO: 331 - protein, 30 aa, synthetic construct
MDSKGSSQKGSRLLLLLVVSNLLLCQGVVS

SEQ ID NO: 332 - protein, 23 aa, synthetic construct
MKAFTSLLCGLGLSTTLAKAISL

SEQ ID NO: 333 - protein, 251 aa, synthetic construct
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYKGGSGSAGSGGSS

SEQ ID NO: 334 - protein, 387 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGR

(19) United States
(12) Patent Application Publication
(10) Pub. No.: US 2024/0271170 A1
(43) Pub. Date: Aug. 15, 2024

(54) POLYNUCLEOTIDES, POLYPEPTIDES, RECOMBINANT CELLS AND METHODS FOR GENERATING ERGOLINES AND PRECURSORS AND METABOLITES THEREOF

(71) Applicant: TryptageniX, Inc., New York, NY (US)

(72) Inventors: Erin Marie Scott, Encinitas, CA (US); Jacob Michael Vogan, San Diego, CA (US); Kirsten Tang, Encinitas, CA (US); James Wade, San Diego, CA (US)

(21) Appl. No.: 18/471,837

(22) Filed: Sep. 21, 2023

Related U.S. Application Data
(60) Provisional application No. 63/376,648, filed on Sep. 22, 2022.

(51) Int. Cl. C12P 17/18; C12N 9/02; C12N 9/04; C12N 9/06; C12N 9/08; C12N 15/81; C12P 17/10; C12R 1/865

(52) U.S. Cl. CPC C12P 17/183; C12N 9/0006; C12N 9/001; C12N 9/0028; C12N 9/0065; C12N 15/81; C12P 17/10; C12R 2001/865; C12Y 101/01332; C12Y 105/01044

(57) ABSTRACT

Provided are non-naturally occurring polynucleotide sequences that encode for polypeptides useful in ergoline biosynthesis, as well as the non-naturally occurring polypeptide sequences. Also provided are recombinant host cell and methods for microbial biosynthesis of an ergoline, or a precursor or metabolite thereof, using the recombinant host cell.

### RELATED APPLICATIONS

This application is related to and claims the benefit of priority of U.S. Provisional Patent Application Ser. No. 63/376,648, filed Sep. 22, 2022, which is incorporated by reference herein in its entirety.

### SEQUENCE LISTING

This application contains a Sequence Listing which is incorporated by reference and is submitted with the filing of this application in computer readable format (CRF) as a file entitled, “66342US01_SeqList.xml”. The Sequence Listing file was created on Sep. 8, 2022 and is 515,778 bytes in size. The CRF file also serves as any paper copy of the Sequence Listing that may be required for purposes of sequence listing submissions.

### FIELD

The disclosure relates to polynucleotides, enzymes, biosynthetic methods, and recombinant microorganisms useful in the production of ergolines and ergot alkaloids such as lysergic acid, and precursors and derivatives thereof.

### BACKGROUND

Ergolines are small alkaloid molecules derived from fungi. The classical example of an ergoline producing fungus is Claviceps purpurea. Fungal pathogens of grasses often have the necessary biosynthetic genes to make ergot type molecules, especially those fungi that parasitize cereal grains.

The ergot family of molecules, also referred to as ergot alkaloids or ergolines, such as those derived from Claviceps purpurea, have established medical uses including, for example, the use of ergotamine in the treatment of acute migraine attacks. Recent interest in ergolines and their potential use in medical applications has expanded to mental health disorders including alcoholism, drug addiction, anxiety, and depression. Ergolines of particular interest in the field include molecules produced by fungi, and derivatives of molecules, which can exhibit desirable therapeutic properties and/or clinical characteristics relative to the naturally-produced ergoline(s).

Some existing methods for the synthesis of ergolines rely on industrial chemical synthesis, which is poorly scalable and environmentally unfriendly (large volumes of hazardous solvents and waste products). Some synthetic methods rely on starting material derived from the original fungus, which can be difficult to growth in amounts required for commercial manufacture. Also, as many ergot alkaloids are naturally produced by organisms, biosynthetic approaches have been explored. However, these biosynthetic approaches have generally been unsuccessful, typically capable of producing precursor compounds only in very low yields (see, e.g., Lu, Jia-Tian, et al., “Total Synthesis of (−)-Chanoclavine I and an Oxygen-Substituted Ergoline Derivative.” J Org Chem 82, 15 (2017): 7774-7782).

Accordingly, there is a need for compositions and methods that provide for improved production of ergot alkaloid molecules in a single recombinant microorganism. The disclosure provides polynucleotides, amino acid sequences (i.e., enzymes), recombinant cells and biosynthetic methods that produce ergolines, including ergoline precursors, metabolites, and derivatives thereof, in good quantity and yield to provide advantages in scale, reliability, reproducibility and environmentally-friendly production, relative to existing production methods.

### SUMMARY

In an aspect the disclosure provides for polynucleotide sequences that encode amino acid sequences (e.g., enzymes, transporters, etc.) that are useful in the biosynthesis of an ergoline. In embodiments of this aspect the polynucleotide sequence comprises a gene that encodes a polypeptide that is associated with ergot alkaloid synthesis (i.e., ergolines, and precursors and metabolites thereof).

In some further embodiments, the polynucleotide that encodes a polypeptide associated with ergot alkaloid synthesis comprises a sequence that is a modified ergot alkaloid synthesis (EAS) gene from an EAS gene cluster. In some embodiments, the polynucleotide sequence comprises any one or more, or various combinations of, an EAS oxidase gene comprising an EASE gene or an EASM gene; an EAS catalase gene comprising an EASC gene; an EAS dehydrogenase gene comprising an EASD gene or an EASG gene; an EAS reductase gene comprising an EASA gene; an EAS acetylase gene comprising an EASN gene; an EAS transferase gene comprising an EASL gene or an EASF gene; an EAS dioxygenase gene comprising an EASH gene; an EAS monooxygenase gene comprising an EASO gene; or an EAS hydrolase gene comprising an EASP gene; or a combination of any two or more EAS genes.

In some further embodiments, the polynucleotide encodes a polypeptide that is useful in the biosynthesis of an ergoline but is not an EAS cluster gene. In such embodiments the polynucleotide sequence can encode for polypeptides having a wide variety of biological activity including, for example, a polypeptide associated with electron transport, a polypeptide associated with metabolic processes, an oxidase, a reductase, an oxygenase (e.g., dioxygenase and/or monooxygenase), a phosphorylase, a peptide synthetase, a halogenase, a transferase (e.g., glycosyltransferase, prenyltransferase), a synthetase (e.g., tryptophan synthetase) or other polypeptides having functional utility in the biosynthesis of an ergoline. In some embodiments the polynucleotide sequence encodes a cytochrome P450 (CYP), a cytochrome reductase (CPR), a cytochrome B (CYB), an oxidase comprising CLOA, a lysergyl peptide synthetase (LPS), a halogenase (HAL), a tryptophan synthetase (TRPS), a prenyltransferase, (dmaW), or a glycosyltransferase (UGT), or combinations thereof. In such further embodiments, one or more of the polynucleotides can be used in combination with one or more polynucleotides that encodes an EAS polypeptide, as described herein.

In some embodiments, the polynucleotide sequence comprises an EASE gene comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 or a sequence having about 95% sequence identity to any one of SEQ ID NOs 1-11.

In some embodiments, the polynucleotide sequence comprises an EASC gene comprising SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19 or a sequence having about 95% sequence identity to any one of SEQ ID NOs 12-19.

In some embodiments, the polynucleotide sequence comprises an EASD gene comprising SEQ D NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29 or a sequence having about 95% sequence identity to any one of SEQ ID NOs 20-29.

In some embodiments, the polynucleotide sequence comprises an EASA gene comprising SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, or SEQ ID NO: 38, or a sequence having about 95% sequence identity to any one of SEQ ID NOs 30-38.

In embodiments, the polynucleotide sequence comprises an EASG gene comprising SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, or SEQ ID NO: 47 or a sequence having about 95% sequence identity to any one of SEQ ID NOs 39-47.

In embodiments, the polynucleotide sequence comprises an EASM gene comprising SEQ ID NO: 90, SEQ ID NO: 91, SEQ ID NO: 92, SEQ ID NO: 93, or SEQ ID NO: 94 or a sequence having about 95% sequence identity to any one of SEQ ID NOs 90-94.

In embodiments, the polynucleotide sequence comprises an EASN gene comprising SEQ ID NO: 95, SEQ ID NO: 96, SEQ ID NO: 97, SEQ ID NO: 98, or SEQ ID NO: 99 or a sequence having about 95% sequence identity to any one of SEQ ID NOs 95-99.

In embodiments, the polynucleotide sequence comprises an EASL gene comprising SEQ ID NO: 100, SEQ ID NO: 101, SEQ ID NO: 102, SEQ ID NO: 103, SEQ ID NO: 104, or SEQ ID NO: 105 or a sequence having about 95% sequence identity to any one of SEQ ID NOs 100-105.

In embodiments, the polynucleotide sequence comprises an EASH gene comprising SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, or SEQ ID NO: 114 or a sequence having about 95% sequence identity to any one of SEQ ID NOs 106-114.

In embodiments, the polynucleotide sequence comprises an EASO gene comprising SEQ ID NO: 115, SEQ ID NO: 116, SEQ ID NO: 117, SEQ ID NO: 118, SEQ ID NO: 119, SEQ ID NO: 120, SEQ ID NO: 121, or SEQ ID NO: 122 or a sequence having about 95% sequence identity to any one of SEQ ID NOs 115-122.

In embodiments, the polynucleotide sequence comprises an EASP gene comprising SEQ ID NO: 123, SEQ ID NO: 124, SEQ ID NO: 125, SEQ ID NO: 126, SEQ ID NO: 127, SEQ ID NO: 128, SEQ ID NO: 129, or SEQ ID NO: 130, or a sequence having about 95% sequence identity to any one of SEQ ID NOs 123-130.

In embodiments, the polynucleotide sequence comprises a CLOA gene comprising SEQ ID NO: 48, SEQ ID NO: 49, SEQ ID NO: 50, SEQ ID NO: 51, SEQ ID NO: 52, or SEQ ID NO: 53 or a sequence having about 95% sequence identity to any one of SEQ ID NOs 48-53.

In embodiments, the polynucleotide sequence comprises a CYP gene comprising SEQ ID NO: 54, SEQ ID NO: 55, SEQ ID NO: 56, SEQ ID NO: 57, or SEQ ID NO: 58 or a sequence having about 95% sequence identity to any one of SEQ ID NOs 54-58.

In embodiments, the polynucleotide sequence comprises a CPR gene comprising SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 71 or a sequence having about 95% sequence identity to any one of SEQ ID NOs 59-71.

In embodiments, the polynucleotide sequence comprises a CYB gene comprising SEQ ID NO: 72, SEQ ID NO: 73, SEQ ID NO: 74, SEQ ID NO: 75, SEQ ID NO: 76, SEQ ID NO: 77, SEQ ID NO: 78, or SEQ ID NO: 79 or a sequence having about 95% sequence identity to any one of SEQ ID NOs 72-79.

In embodiments, the polynucleotide sequence comprises an LPS gene comprising SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, or SEQ ID NO: 89 or a sequence having about 95% sequence identity to any one of SEQ ID NOs 80-89.

In embodiments, the polynucleotide sequence comprises a HAL gene comprising SEQ ID NO: 131, SEQ ID NO: 132, SEQ ID NO: 133, or SEQ ID NO: 134 or a sequence having about 95% sequence identity to any one of SEQ ID NOs 131-134.

In embodiments, the polynucleotide sequence comprises a UGT gene comprising SEQ ID NO: 135, SEQ ID NO: 136, SEQ ID NO: 137, or SEQ ID NO: 138 or a sequence having about 95% sequence identity to any one of SEQ ID NOs 135-138.

In any of the embodiments relating to polynucleotides, the nucleotide sequence encodes an amino acid sequence comprising an EASE of SEQ ID NOs 168-178; an EASC of SEQ ID NOs 179-186; an EASD of SEQ ID NOs 187-196; an EASA of SEQ ID NOs 197-205; an EASG of SEQ ID NOs 206-214; an EASM of SEQ ID NOs 257-261; an EASN of SEQ ID NOs 262-266; an EASL of SEQ ID NOs 267-272; an EASH of SEQ ID NOs 273-281; an EASO of SEQ ID NOs 282-289; an EASP of SEQ ID NOs 290-297; a CLOA of SEQ ID NOs 215-220; a CYP of SEQ ID NOs 221-225; a CPR of SEQ ID NOs 226-238; a CYB of SEQ ID NOs 239-246; an LPS of SEQ ID NOs 247-256; a HAL of SEQ ID NOs 298-301; or a UGT of SEQ ID NOs 302-305.

In some embodiments relating to polynucleotide and amino acid sequences, the disclosure provides for sequence identity between a sequence in accordance with the embodiments of the disclosure and those that have a percent identity to the disclosed sequences. In embodiments, sequences having a percent identity to a sequence described herein also possesses an equivalency to the disclosed sequences (e.g., structural and/or a functional equivalency) and can encompass sequences that comprise at least 85%, 86%, 87%, 88%, 89%, 90%, 91%, 92%, 93%, 94%, 95%, 96%, 97%, 98%, or 99% sequence identity. “Sequence identity” refers to the percentage of nucleotide or amino acid residues that are identical to the sequences that are compared.

In another aspect, the disclosure provides an expression vector comprising anyone or more of the polynucleotide sequences described herein, and an operatively-linked promoter sequence that allows for expression of the one or more polynucleotide sequences in a cell.

In another aspect the disclosure provides for amino acid sequences (e.g., enzymes, transporters, etc.) that are useful in the biosynthesis of an ergoline. In some embodiments, amino acid sequence comprises a modified sequence of a polypeptide (e.g., protein, enzyme, etc.) from an ergot alkaloid synthesis (EAS) cluster. In some embodiments, the amino acid sequence comprises an EAS oxidase comprising an EASE or an EASM; an EAS catalase comprising an EASC; an EAS dehydrogenase comprising an EASD or an EASG; an EAS reductase comprising an EASA; an EAS acetylase comprising an EASN; an EAS transferase comprising an EASL or an EASF; an EAS dioxygenase comprising an EASH gene; an EAS monooxygenase comprising an EASO gene; or an EAS hydrolase comprising an EASP gene; or a combination of any two or more EAS polypeptides.

In some further embodiments the amino acid sequence useful in the biosynthesis of an ergoline is not a protein derived from an EAS cluster. In such embodiments, the polypeptide can have a wide variety of biological activity including, for example, a polypeptide associated with cellular metabolic processes, a polypeptide associated with electron transport an oxidase, a reductase, an oxygenase (e.g., dioxygenase and/or monooxygenase), a phosphorylase, a peptide synthetase, a halogenase, a transferase (e.g., glycosyltransferase), a membrane transporter, a synthetase (e.g., tryptophan synthetase) or other polypeptides having a functional utility in the biosynthesis of an ergoline. In some embodiments the polypeptide sequence comprises a cytochrome P450 (CYP), a cytochrome reductase (CPR), a cytochrome B (CYB), an oxidase comprising CLOA, a lysergyl peptide synthetase (LPS), a halogenase (HAL), a tryptophan synthetase (TRPS), or a glycosyltransferase (UGT), or combinations thereof. In such further embodiments one or more of the polypeptides can be used in combination with one or more polypeptides that comprise an EAS cluster polypeptide, as described herein.

In some embodiments, the amino acid sequence comprises an EASE oxidase, an EASC catalase, an EASD chanoclavine-I dehydrogenase, an EASA chanoclavine reductase, an EASG dehydrogenase, an EASM festuclavine oxidase, an EASN acetylase, an EASL fumigaclavine prenyltransferase, an EASH dioxygenase, an EASO monooxygenase, or an EASP hydrolase, or any combination of two of more amino acid sequences.

In some embodiments, the amino acid sequence comprises an EASE comprising SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, or SEQ ID NO: 178 or a sequence having about 90% sequence identity to any one of SEQ ID NOs 168-178.

In some embodiments, the amino acid sequence comprises an EASC comprising SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 or a sequence having about 90% sequence identity to any one of SEQ ID NOs 179-186.

In some embodiments, the amino acid sequence comprises an EASD comprising SEQ D NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, or SEQ ID NO: 196 or a sequence having about 90% sequence identity to any one of SEQ ID NOs 187-196.

In some embodiments, the amino acid sequence comprises an EASA comprising SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, or SEQ ID NO: 205, or a sequence having about 90% sequence identity to any one of SEQ ID NOs 197-205.

In some embodiments, the amino acid sequence comprises an EASG comprising SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, or SEQ ID NO: 214 or a sequence having about 90% sequence identity to any one of SEQ ID NOs 206-214.

In some embodiments, the amino acid sequence comprises an EASM comprising SEQ ID NO: 257, SEQ ID NO: 258, SEQ ID NO: 259, SEQ ID NO: 260, or SEQ ID NO: 261 or a sequence having about 90% sequence identity to any one of SEQ ID NOs 257-261.

In some embodiments, the amino acid sequence comprises an EASN comprising SEQ ID NO: 262, SEQ ID NO: 263, SEQ ID NO: 264, SEQ ID NO: 265, or SEQ ID NO: 266 or a sequence having about 90% sequence identity to any one of SEQ ID NOs 262-266.

In some embodiments, the amino acid sequence comprises an EASL comprising SEQ ID NO: 267, SEQ ID NO: 268, SEQ ID NO: 269, SEQ ID NO: 270, SEQ ID NO: 271, or SEQ ID NO: 272 or a sequence having about 90% sequence identity to any one of SEQ ID NOs 267-272.

In some embodiments, the amino acid sequence comprises an EASH comprising SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281 or a sequence having about 90% sequence identity to any one of SEQ ID NOs 273-281.

In some embodiments, the amino acid sequence comprises an EASO comprising SEQ ID NO: 282, SEQ ID NO: 283, SEQ ID NO: 284, SEQ ID NO: 285, SEQ ID NO: 286, SEQ ID NO: 287, SEQ ID NO: 288, or SEQ ID NO: 289 or a sequence having about 90% sequence identity to any one of SEQ ID NOs 282-289.

In some embodiments, the amino acid sequence comprises an EASP comprising SEQ ID NO: 290, SEQ ID NO: 291, SEQ ID NO: 292, SEQ ID NO: 293, SEQ ID NO: 294, SEQ ID NO: 295, SEQ ID NO: 296, or SEQ ID NO: 297, or a sequence having about 90% sequence identity to any one of SEQ ID NOs 290-297.

In some embodiments, the amino acid sequence comprises an EASA comprising SEQ ID NO: 215, SEQ ID NO: 216, SEQ ID NO: 217, SEQ ID NO: 218, SEQ ID NO: 219, or SEQ ID NO: 220 or a sequence having about 90% sequence identity to any one of SEQ ID NOs 215-220.

In some embodiments, the amino acid sequence comprises a CYP comprising SEQ ID NO: 221, SEQ ID NO: 222, SEQ ID NO: 223, SEQ ID NO: 224, or SEQ ID NO: 225 or a sequence having about 90% sequence identity to any one of SEQ ID NOs 221-225.

In some embodiments, the amino acid sequence comprises a CPR comprising SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, or SEQ ID NO: 238 or a sequence having about 90% sequence identity to any one of SEQ ID NOs 226-238.

In some embodiments, the amino acid sequence comprises a CYB comprising SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246 or a sequence having about 90% sequence identity to any one of SEQ ID NOs 239-246.

In some embodiments, the amino acid sequence comprises an LPS comprising SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, or SEQ ID NO: 256 or a sequence having about 90% sequence identity to any one of SEQ ID NOs 247-256.

In some embodiments, the amino acid sequence comprises a HAL comprising SEQ ID NO: 298, SEQ ID NO: 299, SEQ ID NO: 300, or SEQ ID NO: 301 or a sequence having about 90% sequence identity to any one of SEQ ID NOs 298-301.

In some embodiments, the amino acid sequence comprises a UGT comprising SEQ ID NO: 302, SEQ ID NO: 303, SEQ ID NO: 304, or SEQ ID NO: 305 or a sequence having about 90% sequence identity to any one of SEQ ID NOs 302-305.

In another aspect, the disclosure provides a recombinant cell comprising any one or more of the polynucleotide sequences, and/or any one or more of the polypeptide sequences described herein. Suitably, a recombinant cell comprising any of the polynucleotide sequences is capable of expressing a functional amino acid sequence (e.g., polypeptide, enzyme, etc.) encoded by the polynucleotide sequence(s).

In some embodiments of this aspect the recombinant cell comprises an EASE polynucleotide sequence and an EASC polynucleotide sequence. In some further embodiments, the recombinant cell can further comprise an EASD polynucleotide sequence.

In some embodiments, the recombinant cell comprises a combination of at least two of an EASG polynucleotide sequence, an EASA polynucleotide sequence, and an EASD polynucleotide sequence. In some embodiments, the recombinant cell comprises an EASG polynucleotide sequence, an EASA polynucleotide sequence, and an EASD polynucleotide sequence.

In some embodiments, the recombinant cell comprises an EASE polynucleotide sequence and an EASC polynucleotide sequence in combination with at least two of an EASG polynucleotide sequence, an EASA polynucleotide sequence, and an EASD polynucleotide sequence. In some embodiments, the cell comprises an EASE, an EASC, an EASG, an EASA, and an EASD polynucleotide sequence.

In yet further embodiments, the recombinant cell according to the above aspects and embodiments can further comprise one or both of an EASM polynucleotide sequence or a CLOA polynucleotide sequence. In some embodiments, the recombinant cell comprises both an EASM polynucleotide sequence and a CLOA polynucleotide sequence. In further embodiments, the recombinant cell further comprises at least one of an LPS polynucleotide sequence, an EASO polynucleotide sequence, an EASP polynucleotide sequence, or an EASH polynucleotide sequence. In some further embodiments, the recombinant cell further comprises an LPS polynucleotide sequence, an EASO polynucleotide sequence, an EASP polynucleotide sequence, and an EASH polynucleotide sequence.

In some other further embodiments, the recombinant cell according to the above aspects and embodiments can further comprise one or more of an EASM polynucleotide sequence, an EASN polynucleotide sequence, or an EASL polynucleotide sequence. In some embodiments, the recombinant cell comprises an EASM polynucleotide sequence, an EASN polynucleotide sequence, and an EASL polynucleotide sequence.

In any of the above aspects and embodiments relating to a recombinant cell, the cell can be fungal cell or a microbial cell. In some embodiments, a microbial cell can be a bacterial cell (e.g., E. coli, or the like) or a yeast cell (e.g., Saccharomyces or Pichia, or the like). In some embodiments, the recombinant cell comprises a further genetic modification that improves the cells ability to produce an ergoline, or a precursor or metabolite thereof.

In another aspect, the disclosure provides a method for the biosynthesis of an ergoline, or a precursor or metabolite thereof, the method comprising culturing a recombinant cell in accordance with the aspects and embodiments of the disclosure under conditions that allow for ergoline biosynthesis.

In some embodiments, the method provides for the biosynthesis of chanoclavine or chanoclavine aldehyde.

In some embodiments, the method provides for the biosynthesis of agroclavine.

In some embodiments, the method provides for the biosynthesis of festuclavine.

In some embodiments, the method provides for the biosynthesis of elymoclavine or dihydro elymoclavine.

In some embodiments, the method provides for the biosynthesis of paspalic aldehyde, dihydro paspalic aldehyde, or paspalic acid.

In some embodiments, the method provides for the biosynthesis of lysergic acid or dihydro lysergic acid.

In some embodiments, the method provides for the biosynthesis of lysergic acid hydroxyethylamide, dihydro lysergic acid hydroxyethylamide, lysergic acid amide, dihydro lysergic acid amide, lysergic acid diethylamide, or dihydro lysergic acid diethylamide.

In some embodiments, the method provides for the biosynthesis of fumigaclavine A, fumigaclavine B, or fumigaclavine C.

Other aspects and embodiments falling within the scope and spirit of the disclosure will be apparent to those of skill in the art in light of the following description and illustrative examples.

### BRIEF DESCRIPTION OF DRAWINGS

FIG. 1 depicts the chemical structures and ring numbering convention for ergolines, precursor molecules, and example embodiments of subclasses of ergolines, exemplifying (A) indole, (B) L-tryptophan, (C) the ergoline ring system, (D) clavines, (E) ergoamides, and (F) ergopeptines.

FIG. 2 depicts the ergoline biosynthetic pathway beginning with (A) the conversion of tryptophan through DMAT intermediates to provide the 3-ringed chanoclavines that undergo further cyclization to the 4-ringed systems, agroclavine or festuclavine; with (B) agroclavine leading to the biosynthesis of lysergic acid; and (C) festuclavine leading to the biosynthesis of dihydro lysergic acid.

FIG. 3 depicts further ergoline biosynthesis reactions including (A) the conversion of lysergic acid to lysergic acid diethylamide; (B) the conversion of dihydro lysergic acid to dihydro lysergic acid diethylamide; (C) the conversion of festuclavine to fumigaclavines (i.e., fumigaclavines A, B, and C); and (D) illustrates stereoisomers within the scope of example embodiments of the disclosure, including iso-forms of lysergic acid amide, dihydro lysergic acid amide, and fumigaclavine A).

FIG. 4 depicts example embodiments of synthesis that can generate ergoline analogs through incorporating substituted precursor molecules, where (A) depicts generating substituted tryptophan analogs by enzymatic reaction of a substituted indole with serine, in the presence of a tryptophan synthase such as TrpS; and where (B) by utilizing and incorporating into a biosynthetic pathway an exogenous substituted tryptophan, and/or (C) a substituted ergoline.

FIG. 5 depicts example embodiments of modified host organisms in accordance with the disclosure that express membrane transporters and cellular gene combinations sufficient (A) for biosynthesis of ergolines (e.g., chanoclavine, agroclavine, festuclavine, lysergic acid, etc.) from initial precursors; and (B) for biosynthesis of ergolines and continuing through to biosynthesis of lysergic acid amide and derivatives thereof. The identified genes, including various membrane-bound transporters or modified transporters, can be expressed in various combinations in accordance with example embodiments of the disclosure, to provide for synthesis of one or more ergolines (including precursors and derivatives thereof), through any one or more steps of the ergoline biosynthetic pathways described herein.

FIG. 6 depicts example embodiments of biosynthetic strategies, in accordance with the disclosure, that can produce and/or act on ergoline pathway products. The biosynthesis can incorporate co-cultures and bioconversions that can include a plurality of microbes, including modified host organisms according to the aspects and embodiments of the disclosure. (A) depicts an example embodiment wherein ergoline pathway products are converted by co-culture of two or more organisms, wherein an organism produces an upstream ergoline precursor and a second organism converts the upstream ergoline precursor to a downstream ergoline compound. (B) depicts an example embodiment wherein a bioconversion wherein an organism produces an upstream ergoline precursor and a cell lysate, either crude or purified (optionally a cell-free lysate), from a lysed co-cultured microbe acts on the upstream ergoline precursor to convert it to a downstream ergoline compound. (C) depicts an example embodiment wherein a lysate from an organism producing an upstream ergoline precursor is acted upon by a second organism that converts the upstream ergoline precursor to a downstream ergoline compound. (D) depicts an example embodiment wherein lysates from two or more co-cultured organisms, one lysate from a cell producing an upstream ergoline precursor and another lysate from a cell that converts the upstream ergoline precursor to a downstream ergoline compound.

FIG. 7 depicts an example HPLC chromatogram and UV-vis spectrum for a fermentation-derived ergoline pathway product, chanoclavine, generated in accordance with the example embodiments of the disclosure. Control HPLC and UV-vis data for a negative control and positive (analytical chanoclavine standard) are provided for comparison.

FIG. 8 depicts LC-MS detection of a fermentation-derived ergoline pathway product, agroclavine, generated in accordance with the example embodiments of the disclosure. Negative controls (blank media, strain not expressing ergoline pathway genes) and a positive control (agroclavine analytical standard) are overlayed for comparison and demonstrate successful agroclavine production.

FIG. 9 depicts a comparison between the mass spectrum fragmentation pattern of fermentation-derived agroclavine and the agroclavine analytical standard.

FIG. 10A-10TTTT identifies sequences in accordance with example embodiments of the disclosure.

### DETAILED DESCRIPTION

In a general sense, the disclosure provides for novel polynucleotide sequences that encode proteins and enzymes such as, for example, EAS sequences that are useful in production of recombinant cells and biosynthetic processes for the production of ergolines. As such, the disclosure provides polynucleotide sequences, enzymes and/or regulatory protein sequences, recombinant cells and methods for the biosynthetic production of ergolines that comprise any one or combination of a chanoclavine synthase (EasE), a chanoclavine reductase (EasA), a catalase (EasC), a dehydrogenase (EasG), a chanoclavine-I dehydrogenase (EasD), an ergoline oxygenase (CloA), an acetylase (EasN), a festuclavine oxidase (EasM), a fumigaclavine prenyltransferase (EasL), a dioxygenase (EasH), a monooxygenase (EasO), a hydrolase (EasP), and/or a lysergyl peptide synthetase (LPS), as well as one or more associated halogenase (HAL), glycosyltransferase (UGT), tryptophan synthetase (TrpS), and/or cytochromes (P450/oxidases, reductases) such as CYP P450 (CYP), a cytochrome reductase (CPR), and/or a cytochrome B (CYB).

### Abbreviations and Definitions

Unless defined otherwise, all technical and scientific terms used herein have the same meaning as commonly understood by one of ordinary skill in the art to which the disclosure belongs. A number of terms and abbreviations appear throughout the disclosure and, unless otherwise indicated, should be understood to have the definitions that follow.

A “nucleic acid” or “polynucleotide” sequence are used herein to refer to deoxyribonucleotides or ribonucleotides and polymers thereof in either single- or double-stranded form. The terms encompass nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The terms “nucleic acid primer,” “nucleic acid probe,” and “oligonucleotide” are all used herein to refer to a short nucleic acid sequence, which may comprise or consist of a fragment of a longer polynucleotide sequence. Oligonucleotides, nucleic acid primers, and/or nucleic acid probes can be DNA, RNA, or a hybrid thereof, or chemically modified analogs or derivatives thereof and are typically single-stranded. However, they can also be double-stranded having two complementing strands that can be separated (e.g., melted) under denaturating conditions (e.g., stringent, moderately stringent, or highly stringent conditions). In some embodiments an oligonucleotide, primer, and/or probe has a length of from about 8 nucleotides to about 200 nucleotides, or from about 12 nucleotides to about 100 nucleotides, or from about 18 to about 50 nucleotides. In embodiments, oligonucleotides, primers, and/or probes can be labeled with detectable markers or modified in any conventional manners for various molecular biological applications (e.g., to inhibit or prevent degradation).

The terms “amino acid sequence,” “polypeptide,” “protein,” and “peptide” as used herein all refer to a sequence of amino acid residues linked by peptide bonds or modified peptide bonds. The amino acid sequence can be of any length of greater than two amino acids. Polypeptides can include modified forms of the sequence, such as naturally occurring or synthetically generated post-translational modifications, or modifications to the chemical structure of one or more amino acid residues. Non-limiting examples of modified forms include glycosylated sequences, phosphorylated sequences, myristoylated sequences, palmitoylated sequences, ribosylated sequences, acetylated sequences, and the like. Modifications can also include intra- or inter-molecular crosslinking or covalent attachments to moieties such as lipids, flavin, biotin, polyethylene glycol or derivatives thereof, and the like. In addition, modifications may also include protein cyclization, branching of the amino acid chain, and cross-linking of the protein. Further, amino acids other than the naturally-encoded twenty amino acids may also be included in a polypeptide.

The polypeptide sequences or polynucleotide sequences can be isolated and/or purified, both of which refer to a polypeptide (or a polynucleotide) that is substantially separated from other cellular components (i.e., proteins, DNA, RNA, lipids, membranes, cell debris) of the organism in which the sequence is produced (e.g., 30%, 40%, 50%, 60%, 70%, 80%, 90%, 95%, 96%, 97%, 98%, 99%, 100% free of contaminants).

As used herein, a “conservative amino acid substitution” refers to an amino acid substitution in a polypeptide sequence wherein the substituted amino acid(s) has similar characteristics to the amino acid in the native sequence, for example charge, hydrophobicity and/or hydrophilicity profile, polarity, size, and the like. Non-limiting examples of conservative amino acid substitutions are Ser for Ala, Thr, or Cys; Lys for Arg; Gln for Asn, His, or Lys; His for Asn; Glu for Asp or Lys; Asn for His or Gln; Asp for Glu; Pro for Gly; Leu for Ile, Phe, Met, or Val; Val for Ile or Leu; Ile for Leu, Met, or Val; Arg for Lys; Met for Phe; Tyr for Phe or Trp; Thr for Ser; Trp for Tyr; and Phe for Tyr. Non-natural amino acids can also serve as a conservative amino acid substitution for a naturally occurring amino acid.

The term “functional variant” refers to a recombinant biological sequence that is structurally different from a naturally occurring sequence and capable of performing the same function as the naturally occurring sequence. For example, a functional variant of an ergoline pathway gene or enzyme comprises a nucleotide and/or amino acid sequence that is altered by one or more nucleotides and/or amino acids compared to the native ergoline pathway gene or enzyme sequences, and is capable of performing the function of the native or parent ergoline pathway gene or enzyme. In embodiments, a modification to the native or parent sequence may provide for the same or improved functional activity and reaction parameters without altering the underlying function of the native biological sequence. Functional variants may comprise conservative sequence substitutions, sequence additions, and sequence deletions. The sequence modifications can be introduced by standard techniques known in the art, such as site-directed mutagenesis and random PCR-mediated mutagenesis, and may comprise natural as well as non-natural nucleotides and amino acids, and/or analogs thereof. In some embodiments, recombinant biological sequences, including functional variants, comprise amino acid analogs (e.g. amino acids other than the 20 amino acids encoded by DNA or RNA) and/or labeled amino acids and amino acid analogs comprising, for example, fluorescent dyes, radioisotopes, electron dense agents, and the like.

A “recombinant” nucleic acid or amino acid sequence is a nucleic acid or polypeptide produced by recombinant DNA technology, e.g., as described in Green and Sambrook (2012). The terms “recombinant,” “heterologous,” and “exogenous,” can be used interchangeably herein and, when referring to polynucleotides, mean a polynucleotide (e.g., a DNA or RNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, the terms when referring to polypeptides, means a polypeptide or amino acid sequence that originates from a source foreign to the particular host cell or, if from the same source, is modified from its original form. As such, recombinant DNA molecules can be expressed in a host cell to produce a recombinant polypeptide.

The terms “transformed,” “transgenic,” and “recombinant,” when used with reference to host cells typically refer to an isolated cell or a cell in culture, such as a plant, fungal, or microbial (e.g. bacterial or yeast) cell, into which a heterologous polynucleotide has been introduced or a heterologous amino acid sequence is expressed. The polynucleotide can be integrated into the genome of the host cell, or it can be present as an extrachromosomal molecule. Such an extrachromosomal molecule can be auto-replicating, as discussed herein. Transformed cells, tissues, or subjects are understood to encompass not only the end-product of a transformation process, but also transgenic progeny thereof.

The terms “plasmid,” “vector,” and “cassette” (e.g., transformation cassette, expression vector, or expression cassette) generally refer to an extra-chromosomal element that comprises nucleic acid sequences (e.g., genes, promoters, regulatory elements (inducers, repressors, etc.) and the like) which are not part of the central metabolism of the cell, and can be circular, double-stranded DNA molecules. Such elements may be autonomously replicating sequences, genome integrating sequences, phage or nucleotide sequences (linear or circular) of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Merely for purposes of clarity, a “transformation cassette” refers to a vector containing a foreign gene and having elements in addition to the foreign gene that facilitate transformation of a particular host cell. An “expression cassette” or “expression vector” refers to a vector containing a foreign gene and having elements in addition to the foreign gene that allow for expression and/or enhanced expression of that gene in a foreign host. Thus, the terms can refer to a nucleic acid molecule capable of transporting another nucleic acid molecule to which it has been linked. A non-limiting example of an expression vector includes a gene encoding an enzyme with a promoter that is functional in yeast, where the promoter and gene are oriented such that the promoter drives expression of the enzyme in the yeast cell. A non-limiting example of a vector capable of extra-chromosomal replication is an episome.

A “linker” refers to a short amino acid sequence that separates multiple domains of a polypeptide. In some embodiments, the linker prohibits energetically or structurally unfavorable interactions between the discrete domains.

A recombinant gene can be “codon optimized” when its nucleotide sequence is modified to accommodate codon bias of the host organism, typically to improve gene expression and increase translational efficiency of the gene.

As used herein a “coding sequence” generally refers to a DNA sequence that encodes for a specific amino acid sequence.

A “regulatory sequence” is generally used to refer to a polynucleotide sequence located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

A “promoter” refers to a DNA sequence capable of controlling the expression of a coding sequence or functional RNA. Commonly, a coding sequence is oriented or located 3′ to a promoter sequence. Promoters may be derived in their entirety from a native gene, or be composed of different elements derived from different natural promoters, or comprise synthetic DNA segments. Different promoters may direct the expression of a gene in different cell types, or at different stages of development or cell growth/cycle, or in response to different environmental conditions. Promoters that cause a gene to be expressed in most cell types at most times are commonly referred to as “constitutive promoters.”

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” generally refers to the transcription and stable accumulation of sense (mRNA) or antisense RNA derived from a nucleic acid sequence. “Over-expression” refers to the production of a gene product in transgenic or recombinant organisms that exceeds levels of production in normal or non-transformed organisms.

“Transformation” is used according to its ordinary and customary meaning as understood by a person of ordinary skill in the art, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “recombinant” or “transformed” organisms.

### Polynucleotides

In various aspects the disclosure provides for polynucleotide sequences that encode amino acid sequences (e.g., enzymes, transporters, etc.) that are useful in the biosynthesis of an ergoline. In embodiments of this aspect, the polynucleotide sequence comprises a gene that encodes a polypeptide that is associated with ergot alkaloid synthesis (i.e., ergolines, and precursors and metabolites thereof). In further embodiments, the polynucleotide comprises a sequence that is a modified ergot alkaloid synthesis (EAS) gene from an EAS gene cluster. In some embodiments, the polynucleotide sequence comprises an EAS oxidase gene comprising an EASE gene or an EASM gene; an EAS catalase gene comprising an EASC gene; an EAS dehydrogenase gene comprising an EASD gene or an EASG gene; an EAS reductase gene comprising an EASA gene; an EAS acetylase gene comprising an EASN gene; an EAS transferase gene comprising an EASL gene; an EAS dioxygenase gene comprising an EASH gene; an EAS monooxygenase gene comprising an EASO gene; or an EAS hydrolase gene comprising an EASP gene; or a combination of any two or more EAS genes.

In some further embodiments, the polynucleotide encodes a polypeptide that is useful in the biosynthesis of an ergoline but is not an EAS cluster gene. In such embodiments, the polynucleotide sequence can encode for polypeptides having a wide variety of biological activity including, for example, a polypeptide associated with electron transport, a polypeptide associated with metabolic processes, an oxidase, a reductase, an oxygenase (e.g., dioxygenase and/or monooxygenase), a phosphorylase, a peptide synthetase, a halogenase, a transferase (e.g., glycosyltransferase, prenyltransferase), a synthetase (e.g., tryptophan synthetase) or other polypeptides having functional utility in the biosynthesis of an ergoline. In some embodiments the polynucleotide sequence encodes a cytochrome P450 (CYP), a cytochrome reductase (CPR), a cytochrome B (CYB), an oxidase comprising CLOA, a lysergyl peptide synthetase (LPS), a halogenase (HAL), a tryptophan synthetase (TRPS), or a glycosyltransferase (UGT), or combinations thereof. In such further embodiments, one or more of the polynucleotides can be used in combination with one or more polynucleotides that encodes an EAS polypeptide, as described herein.

In embodiments, the polynucleotide sequence comprises an EASE gene comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 or a sequence having about 90% sequence identity to any one of SEQ ID NOs 1-11.

In embodiments, the polynucleotide sequence comprises an EASC gene comprising SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19 or a sequence having about 90% sequence identity to any one of SEQ ID NOs 12-19.

In embodiments, the polynucleotide sequence comprises an EASD gene comprising SEQ D NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29 or a sequence having about 90% sequence identity to any one of SEQ ID NOs 20-29.

In embodiments, the polynucleotide sequence comprises an EASA gene comprising SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, or SEQ ID NO: 38, or a sequence having about 90% sequence identity to any one of SEQ ID NOs 30-38.

In embodiments, the polynucleotide sequence comprises an EASG gene comprising SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, or SEQ ID NO: 47 or a sequence having about 90% sequence identity to any one of SEQ ID NOs 39-47.

In embodiments, the polynucleotide sequence comprises an EASM gene comprising SEQ ID NO: 90, SEQ ID NO: 91, SEQ ID NO: 92, SEQ ID NO: 93, or SEQ ID NO: 94 or a sequence having about 90% sequence identity to any one of SEQ ID NOs 90-94.

In embodiments, the polynucleotide sequence comprises an EASN gene comprising SEQ ID NO: 95, SEQ ID NO: 96, SEQ ID NO: 97, SEQ ID NO: 98, or SEQ ID NO: 99 or a sequence having about 90% sequence identity to any one of SEQ ID NOs 95-99.

In embodiments, the polynucleotide sequence comprises an EASL gene comprising SEQ ID NO: 100, SEQ ID NO: 101, SEQ ID NO: 102, SEQ ID NO: 103, SEQ ID NO: 104, or SEQ ID NO: 105 or a sequence having about 90% sequence identity to any one of SEQ ID NOs 100-105.

In embodiments, the polynucleotide sequence comprises an EASH gene comprising SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, or SEQ ID NO: 114 or a sequence having about 90% sequence identity to any one of SEQ ID NOs 106-114.

In embodiments, the polynucleotide sequence comprises an EASO gene comprising SEQ ID NO: 115, SEQ ID NO: 116, SEQ ID NO: 117, SEQ ID NO: 118, SEQ ID NO: 119, SEQ ID NO: 120, SEQ ID NO: 121, or SEQ ID NO: 122 or a sequence having about 90% sequence identity to any one of SEQ ID NOs 115-122.

In embodiments, the polynucleotide sequence comprises an EASP gene comprising SEQ ID NO: 123, SEQ ID NO: 124, SEQ ID NO: 125, SEQ ID NO: 126, SEQ ID NO: 127, SEQ ID NO: 128, SEQ ID NO: 129, or SEQ ID NO: 130, or a sequence having about 90% sequence identity to any one of SEQ ID NOs 123-130.

In embodiments, the polynucleotide sequence comprises a CLOA gene comprising SEQ ID NO: 48, SEQ ID NO: 49, SEQ ID NO: 50, SEQ ID NO: 51, SEQ ID NO: 52, or SEQ ID NO: 53 or a sequence having about 90% sequence identity to any one of SEQ ID NOs 48-53.

In embodiments, the polynucleotide sequence comprises a CYP gene comprising SEQ ID NO: 54, SEQ ID NO: 55, SEQ ID NO: 56, SEQ ID NO: 57, or SEQ ID NO: 58 or a sequence having about 90% sequence identity to any one of SEQ ID NOs 54-58.

In embodiments, the polynucleotide sequence comprises a CPR gene comprising SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 71 or a sequence having about 90% sequence identity to any one of SEQ ID NOs 59-71.

In embodiments, the polynucleotide sequence comprises a CYB gene comprising SEQ ID NO: 72, SEQ ID NO: 73, SEQ ID NO: 74, SEQ ID NO: 75, SEQ ID NO: 76, SEQ ID NO: 77, SEQ ID NO: 78, or SEQ ID NO: 79 or a sequence having about 90% sequence identity to any one of SEQ ID NOs 72-79.

In embodiments, the polynucleotide sequence comprises an LPS gene comprising SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, or SEQ ID NO: 89 or a sequence having about 90% sequence identity to any one of SEQ ID NOs 80-89.

In embodiments, the polynucleotide sequence comprises a HAL gene comprising SEQ ID NO: 131, SEQ ID NO: 132, SEQ ID NO: 133, or SEQ ID NO: 134 or a sequence having about 90% sequence identity to any one of SEQ ID NOs 131-134.

In embodiments, the polynucleotide sequence comprises a UGT gene comprising SEQ ID NO: 135, SEQ ID NO: 136, SEQ ID NO: 137, or SEQ ID NO: 138 or a sequence having about 90% sequence identity to any one of SEQ ID NOs 135-138.

In any of the embodiments relating to polynucleotides, the nucleotide sequence encodes an amino acid sequence comprising an EASE of SEQ ID NOs 168-178; an EASC of SEQ ID NOs 179-186; an EASD of SEQ ID NOs 187-196; an EASA of SEQ ID NOs 197-205; an EASG of SEQ ID NOs 206-214; an EASM of SEQ ID NOs 257-261; an EASN of SEQ ID NOs 262-266; an EASL of SEQ ID NOs 267-272; an EASH of SEQ ID NOs 273-281; an EASO of SEQ ID NOs 282-289; an EASP of SEQ ID NOs 290-297; a CLOA of SEQ ID NOs 215-220; a CYP of SEQ ID NOs 221-225; a CPR of SEQ ID NOs 226-238; a CYB of SEQ ID NOs 239-246; an LPS of SEQ ID NOs 247-256; a HAL of SEQ ID NOs 298-301; or a UGT of SEQ ID NOs 302-305.

Nucleic acid sequences in accordance with the disclosure are synthesized and cloned using techniques known in the art. Gene expression can be controlled by inducible or constitutive promoter systems using the appropriate expression vectors. Genes are transformed into an organism using standard yeast or fungus transformation methods to generate modified host strains (i.e., the recombinant host organism). The modified strains express the genes for biosynthetic pathways that generate ergoline pathway products, such as lysergic acid and/or all intermediate ergoline compounds described herein. The ergoline pathway genes herein can be integrated into the genome of the cell or maintained as an episomal plasmid. Recombinant host fermentation samples are: (i) prepared and extracted using a combination of fermentation, dissolution, and purification steps; and (ii) analyzed by HPLC for the presence of directing molecules (e.g., DMA-L-abrine), precursor molecules, intermediate molecules, and target molecules such as chanoclavine or lysergic acid.

The polynucleotides can be used in, or used to generate, modified strains of host cells, which produce ergolines, precursors or metabolites thereof, and can express genes encoding one or more of (i) a chanoclavine synthase, (ii) chanoclavine aldehyde dehydrogenase (iii) agroclavine synthase, (iv) CYP P450s and CPRs that synthesize elymoclavine and/or lysergic acid. (v) peptide synthases for making ergopeptines.

In some embodiments, the nucleic acid sequence (i.e., polynucleotide) encoding a gene or a complementary nucleic acid sequence to such a coding sequence can be codon optimized for production in a selected microorganism. A number of factors can be used in determining a codon-optimized sequence (see, e.g., U.S. Pat. No. 10,435,727). Factors can include, for example, (1) selecting a codon for each amino acid residue in the recombinant polypeptide based on the usage frequency of each codon in the heterologous host cell (e.g., Saccharomyces cerevisiae) genome; (2) removing sequences that provide for restriction sites for enzymes to prevent DNA cleavage; (3) modifying long repeats (e.g., consecutive sequences of 5 or more nucleotide) to prevent low-complexity regions; (4) adding a ribosome binding site to the N-terminus; (5) adding a stop codon; (6) changing nucleotides that encode amino acids susceptible to undesirable post-translational modifications (e.g., changing codons for a surface exposed LYS to an ARG codon to avoid ubiquitination); (7) removing or replacing a localization signal sequence.

In various embodiments, the nucleic acid sequences can further comprise additional sequence encoding amino acids that are not part of the included enzymes or regulatory proteins herein. In some of these embodiments, the additional sequences encode additional amino acids present when the nucleic acid is translated, encoding, for example, a co-folding peptide, as disclosed herein, or an additional protein domain, with or without a linker sequence, creating a fusion protein. Other examples are localization sequences, i.e., signals directing the localization of the folded protein to a specific subcellular compartment or membrane. Additional non-limiting examples are an affibody tag, a localization scaffold, a vacuolar localization tag, a secretion signal, and a histidine tag (e.g., 6×his tag). Additional examples include cleavage sites, such as a TEV protease recognition sequence or 2A-self-cleaving peptides encoded between two or more genes for cleavage of individual proteins post-translation.

In some embodiments, the nucleic acid comprises additional nucleotide sequences that are not translated. Non-limiting examples include promoters, terminators, barcodes, Kozak sequences, targeting sequences, and enhancer elements. In some particular embodiments the polynucleotide sequences comprise a promoter that is functional in yeast, fungi, and bacteria. In embodiments, expression of a gene encoding an enzyme or regulatory protein is controlled by the promoter operably linked to the gene sequence. For a gene to be expressed, a promoter must be present within 1,000 nucleotides upstream of the gene. A gene is generally cloned under the control of a desired promoter. The promoter is placed upstream of the gene in the genome or on an episomal plasmid. The promoter regulates the amount of enzyme expressed in the cell and the timing of expression, or expression in response to external factors such as carbon source.

Any promoter can be utilized to drive the expression of the enzymes and regulatory proteins described herein. Listings of various promoters in organisms such as yeast are readily available (See, e.g., the registry of Standard Biological Parts for yeast at the website: parts.igem.org/Yeast). Several of the exemplary promoters listed in Table 4 below drive strong expression, constant gene expression, medium or weak gene expression, or provide inducible gene expression. Inducible or repressible gene expression is dependent on the presence or absence of a certain molecule (inducer/repressor). For example, the GAL1, GAL7, and GAL10 promoters are activated by the presence of galactose and are repressed by the presence of glucose. The HO promoter is active and drives gene expression only in the presence of the alpha factor peptide. The HXT1 promoter is activated by the presence of glucose while the ADH2 promoter is repressed by the presence of glucose.

TABLE 1
Exemplary promoters

| Constitutive promoters | Constitutive promoters | Inducible/repressible |
|---|---|---|
| (Strong) | (Medium and weak) | promoters |
| TEF1 | STE2 | GAL1 |
| PGK1 | TPI1 | GAL7 |
| PGI1 | PYK1 | GAL10 |
| TDH3 | CYC1 | HO |
| T7 | ADH1 | HXT1 |
| | | ADH2 |
| | | AOX1 |
| | | T5-lac |

As discussed herein, various embodiments of the disclosure provide the nucleic acid sequence as an expression cassette, e.g., a yeast expression cassette. Any yeast expression cassette capable of expressing the enzyme in a yeast cell can be utilized. Additional regulatory elements can also be present in the expression cassette, including restriction enzyme cleavage sites, antibiotic resistance genes, integration sites, auxotrophic selection markers, origins of replication, and degrons.

The expression cassette can be present in a vector that, when transformed into a host cell, either integrates into chromosomal DNA or remains episomal in the host cell. Such vectors are well-known in the art. One non-limiting example of a yeast vector is a yeast episomal plasmid (YEp) that contains the pBluescript II SK(+) phagemid backbone, an auxotrophic selectable marker, yeast and bacterial origins of replication and multiple cloning sites enabling gene cloning under a suitable promoter (see Table 4). Other non-limiting vectors include pRS series plasmids.

Mutations introduced into the DNA can provide enzyme variations that can prevent or promote post translational modifications of the protein. Non-limiting examples of post-translational modifications include phosphorylation, acetylation, methylation, SUMOylation, ubiquitination, proteolytic cleavage, lipidation, prenylation such as farnesylation or myristoylation, glycosylation, nitrosylation and biotinylation.

The nucleic acid sequences can be modified from a gene from any source, e.g., any microorganism, protist, virus, plant, or animal. In some embodiments, the gene encoding an enzyme or regulatory protein is derived from a bacterium. For example, the bacterium can be from phylum Abditibacteriota, including class Abditibacteria, including order Abditibacteriales; phylum Abyssubacteria or Acidobacteria, including class Acidobacteriia, Blastocatellia, Holophagae, Thermoanaerobaculia, or Vicinamibacteria, including order Acidobacteriales, Bryobacterales, Blastocatellales, Acanthopleuribacterales, Holophagales, Thermotomaculales, Thermoanaerobaculales, or Vicinamibacteraceae; phylum Actinobacteria, including class Acidimicrobiia, Actinobacteria, Actinomarinidae, Coriobacteriia, Nitriliruptoria, Rubrobacteria, or Thermoleophilia, including orders Acidimicrobiales, Acidothermales, Actinomycetales, Actinopolysporales, Bifidobacteriales, Nanopelagicales, Catenulisporales, Corunebacteriales, Cryptosporangiales, Frankiales, Geodermatophilales, Glycomycetales, Jiangellales, Micrococcales, Micromonosporales, Nakamurellales, Propionibacteriales, Pseudonocardiales, Sporichthyales, Streptomycetales, Streptosporangiales, Actinomarinales, Coriobacteriales, Eggerthellales, Egibacterales, Egicoccales, Euzebyales, Nitriliruptorales, Gaiellales, Rubrobacterales, Solirubrobacterales, or Thermoleophilales; phylum Aquificae, including class Aquificae, including order Aquificales or Desulfurobacteriales; phylum Armatimonadetes, including class Armatimonadia, including order Armatimonadales, Capsulimonadales, Chthonomonadetes, Chthonomonadales, Fimbriimonadia, or Fimbriimonadales; phylum Aureabacteria or Bacteroidetes, including class Armatimonadia, Bacteroidia, Chitinophagia, Cytophagia, Flavobacteria, Saprospiria or Sphingobacteriia, including order Bacteroidales, Marinilabiliales, Chitinophagales, Cytophagales, Flavobacteriales, Saprospirales, or Sphingopacteriales; phylum Balneolaeota, Caldiserica, Calditrichaeota, or Chlamydiae, including class Balneolia, Caldisericia, Calditrichae, or Chlamydia, including order Balneolales, Caldisericales, Calditrichales, Anoxychlamydiales, Chlamydiales, or Parachlamydiales; phylum Chlorobi or Chloroflexi, including class Chlorobia, Anaerolineae, Ardenticatenia, Caldilineae, Thermofonsia, Chloroflexia, Dehalococcoidia, Ktedonobacteria, Tepidiformia, Thermoflexia, Thermomicrobia, or Sphaerobacteridae, including order Chlorobiales, Anaerolineales, Ardenticatenales, Caldilineales, Chloroflexales, Herpetosiphonales, Kallotenuales, Dehalococcoidales, Dehalogenimonas, Ktedonobacterales, Thermogemmatisporales, Tepidiformales, Thermoflexales, Thermomicrobiales, or Sphaerobacterales; phylum Chrysiogenetes, Cloacimonetes, Coprothermobacterota, Cryosericota, or Cyanobacteria, including class Chrysiogenetes, Coprothermobacteria, Gloeobacteria, or Oscillatoriophycideae, including order Chrysiogenales, Coprothermobacterales, Chroococcidiopsidales, Gloeoemargaritales, Nostocales, Pleurocapsales, Spirulinales, Synechococcales, Gloeobacterales, Chroococcales, or Oscillatoriales; phyla: Eferribacteres, Deinococcus-thermus, Dictyoglomi, Dormibacteraeota, Elusimicrobia, Eremiobacteraeota, Fermentibacteria, or Fibrobacteres, including class Deferribacteres, Deinococci, Dictyoglomia, Elusimicrobia, Endomicrobia, Chitinispirillia, Chitinivibrionia, or Fibrobacteria, including order Deferribacterales, Deinococcales, Thermales, Dictyoglomales, Elusimicrobiales, Endomicrobiales, Chitinspirillales, Chitinvibrionales, Fibrobacterales, or Fibromonadales; phylum Firmicutes, Fusobacteria, Gemmatimonadetes, or Hydrogenedentes, including class Bacilli, Clostridia, Erysipelotrichia, Limnochordia, Negativicutes, Thermolithobacteria, Tissierellia, Fusobacteriia, Gemmatimonadetes, Longimicrobia, including order Bacillales, Lactobacillales, Borkfalkiales, Clostridiales, Halanaerobiales, Natranaerobiales, Thermoanaerobacterales, Erysipelotrichales, Limnochordales, Acidaminococcales, Selenomonadales, Veillonellales, Thermolithobacterales, Tissierellales, Fusobacteriales, Gemmatimonadales, or Longimicrobia; phylum Hydrogenedentes, Ignavibacteriae, Kapabacteria, Kiritimatiellaeota, Krumholzibacteriota, Kryptonia, Latescibacteria, LCP-89, Lentisphaerae, Margulisbacteria, Marinimicrobia, Melainabacteria, Nitrospinae, or Omnitrophica, including class Ignavibacteria, Kiritimatiellae, Krumholzibacteria, Lentisphaeria, Oligosphaeria, or Nitrospinae, including order Ignavibacteriales, Kiritimatiellales, Krumholzibacteriales, Lentisphaerales, Victivallales, Oligosphaerales, or Nitrospinia; phylum Omnitrophica or Planctomycetes, including class Brocadiae, Phycisphaerae, Planctomycetia, or Phycisphaerales, including order Sedimentisphaerales, Tepidisphaerales, Gemmatales, Isosphaerales, Pirellulales, or Planctomycetales; phylum Proteobacteria including class Acidithiobacillia, Alphaproteobacteria, Betaproteobacteria, Lambdaproteobacteria, Muproteobacteria, Deltaproteobacteria, Epsilonproteobacteria, Gammaproteobacteria, Hydrogenophilalia, Oligoflexia, or Zetaproteobacteria, including order Acidithiobacillales, Caulobacterales, Emcibacterales, Holosporales, Iodidimonadales, Kiloniellales, Kopriimonadales, Kordiimonadales, Magnetococcales, Micropepsales, Minwuiales, Parvularculales, Pelagibacterales, Rhizobiales, Rhodobacterales, Rhodospirillales, Rhodothalassiales, Rickettsiales, Sneathiellales, Sphingomonadales, Burkholderiales, Ferritrophicales, Ferrovales, Neisseriales, Nitrosomonadales, Procabacteriales, Rhodocyclales, Bradymonadales, Acidulodesulfobacterales, Desulfarculales, Desulfobacterales, Desulfovibrionales, Desulfurellales, Desulfuromonadales, Myxococcales, Syntrophobacterales, Campylobacterales, Nautiliales, Acidiferrobacterales, Aeromonadales, Alteromonadales, Arenicellales, Cardiobacteriales, Cellvibrionales, Chromatiales, Enterobacterales, Immundisolibacterales, Legionellales, Methylococcales, Nevskiales, Oceanospirillales, Orbales, Pasteurellales Pseudomonadales, Salinisphaerales, Thiotrichales, Vibrionales, Xanthomonadales, Hydrogenophilales, Bacteriovoracales, Bdellovibrionales, Oligoflexales, Silvanigrellales, or Mariprofundales; phylum Rhodothermaeota, Saganbacteria, Sericytochromatia, Spirochaetes, Synergistetes, Tectomicrobia, or Tenericutes, including class Rhodothermia, Spirochaetia, Synergistia, Izimaplasma, or Mollicutes, including order Rhodothermales, Brachyspirales, Brevinematales, Leptospirales, Spirochaetales, Synergistales, Acholeplasmatales, Anaeroplasmatales, Entomoplasmatales, or Mycoplasmatales; phylum Thermodesulfobacteria, Thermotogae, Verrucomicrobia, or Zixibacteria, including class Thermodesulfobacteria, Thermotogae, Methylacidiphilae, Opitutae, Spartobacteria, or Verrucomicrobiae, including order Thermodesulfobacteriales, Kosmotogales, Mesoaciditogales, Petrotogales, Thermotogales, Methylacidiphilales, Opitutales, Puniceicoccales, Xiphinematobacter, Chthoniobacterales, Terrimicrobium, or Verrucomicrobiales.

In other embodiments, the gene encoding the enzyme or regulatory protein is modified from an archaeon. For example, the archaeon can be from: phylum Euryarchaeota, including class Archaeoglobi, Hadesarchaea, Halobacteria, Methanobacteria, Methanococci, Methanofastidiosa, Methanomicrobia, Methanopyri, Nanohaloarchaea, Theionarchaea, Thermococci, or Thermoplasmata, including order Archaeoglobales, Hadesarchaeales, Halobacteriales, Methanobacteriales, Methanococcales, Methanocellales, Methanomicrobiales, Methanophagales, Methanosarcinales, Methanopyrales, Thermococcales, Methanomassiliicoccales, Thermoplasmatales, or Nanoarchaeales; DPANN superphylum, including subphyla Aenigmarcheota, Altiarchaeota, Diapherotrites, Micrarchaeota, Nanoarchaeota, Pacearchaeota, Parvarchaeota, or Woesearchaeota; TACK superphylum, including subphylum Korarchaeota, Crenarchaeota, Aigarchaeota, Geoarchaeota, Thaumarchaeota, or Bathyarchaeota; Asgard superphylum including subphylum Odinarchaeota, Thorarchaeota, Lokiarchaeota, Helarchaeota, orHeimdallarchaeota.

In additional embodiments, the gene encoding the enzyme or regulatory protein is modified from a fungus. For example, the fungus can be from: phyla Chytridiomycota, Basidiomycota, Ascomycota, Blastocladiomycota, Ascomycota, Microsporidia, Basidiomycota, Glomeromycota, Symbiomycota, and Neocallimastigomycota; phylum Ascomycota, including classes and orders Pezizomycotina, Arthoniomycetes, Coniocybomycetes, Dothideomycetes, Eurotiomycetes, Geoglossomycetes, Laboulbeniomycetes, Lecanoromycetes, Leotiomycetes, Lichinomycetes, Orbiliomycetes, Pezizomycetes, Sordariomycetes, Xylonomycetes, Lahmiales, Itchiclahmadion, Triblidiales, Saccharomycotina, Saccharomycetes, Taphrinomycotina, Archaeorhizomyces, Neolectomycetes, Pneumocystidomycetes, Schizosaccharomycetes, Taphrinomycetes; phylum Basidiomycota including subphyla or classes Pucciniomycotina, Ustilaginomycotina, Wallemiomycetes, and Entorrhizomycetes; subphylum Agaricomycotina including classes Tremellomycetes, Dacrymycetes, and Agaricomycetes; phylumSymbiomycota, including class Entorrhizomycota; subphylum Ustilaginomycotina including classes Ustilaginomycetes and Exobasidiomycetes; phylum Glomeromycota including classes Archaeosporomycetes, Glomeromycetes, and Paraglomeromycetes; subphylum Pucciniomycotina including orders and classes: Pucciniomycotina, Cystobasidiomycetes, Agaricostilbomycetes, Microbotryomycetes, Atractiellomycetes, Classiculomycetes, Mixiomycetes, and Cryptomycocolacomycetes; subphylum incertae sedis Mucoromyceta including orders Calcarisporiellomycota and Mucoromycota; phylum Mortierellomyceta including class Mortierellomycota; subphylum incertae sedis Entomophthoromycotina including order Entomophthorales; phylum Zoopagomyceta including classes Basidiobolomycota, Entomophthoromycota, Kickxellomycota, and Zoopagomycotina; subphylum incertae sedis Mucoromycotina including orders Mucorales, Endogonales, and Mortierellales; phylum Neocallimastigomycota including class Neocallimastigomycetes; phylum Blastocladiomycota including classes Physodermatomycetes and Blastocladiomycetes; phylum Rozellomyceta including classes Rozellomycota and Microsporidia; phylum Aphelidiomyceta including class Aphelidiomycota; phylum Chytridiomyceta including classes Chytridiomycetes and Monoblepharidomycetes; phylum Oomycota including classes or orders Leptomitales, Myzocytiopsidales, Olpidiopsidales, Peronosporales, Pythiales, Rhipidiales, Salilagenidiales, Saprolegniales, Sclerosporales, Anisolpidiales, Lagenismatales, Rozellopsidales, and Haptoglossales.

In some embodiments, the gene encoding the enzyme or regulatory protein is modified from any one or more exemplary organisms including, but is not limited to: Acanthurus tractus, Aplysina aerophoba, Bos Taurus, Bufo bufo, Bufotes viridis, Chrysochloris asiatica, Fukomys damarensis, Homo sapiens, Rattus norvegicus, Rhinella marina, Rhinella spinulosa, Schistosoma mansoni, Xenopus laevis, Xenopus tropicalis, Acacia koa, Arabidopsis thaliana, Psilocybe cubensis, Brassica oleracea, Citrus sinensis, Hordeum vulgare, Juglans cinereal, Lophophora williamsii, Nymphaea colorata, Oryza sativa, Ipomoea violaceae, Rivea corymbosa, Argyreia nervosa, Merremia tuberose, Ricinus communis, Solanum lycopersicum, Sorghum bicolor, Theobroma cacao, and Triticum aestivum.

### Enzymes and Amino Acid Sequences

The disclosure provides for non-naturally occurring amino acid sequences (i.e., enzymes or regulatory proteins) comprising a sequence encoded by any of the nucleic acids described above. In some embodiments, the amino acid sequence is 85%, 90%, 95%, 98%, or 100% identical to any one of SEQ ID NOs: 168-305 as disclosed herein. In these embodiments, the enzyme or regulatory protein can be isolated in vitro and used in vitro to provide enzyme activity. Alternatively, the enzyme can be expressed in a recombinant organism as described herein. In some embodiments, the recombinant microorganism for the recombinant production of an amino acid sequence is a bacterium, for example an E. coli. In other embodiments, the recombinant microorganism is a yeast or fungal cell, e.g., a species of Saccharomyces (for example S. cerevisiae), Candida, Pichia, Schizosaccharomyces, Scheffersomyces, Blakeslea, Rhodotorula, Aspergillus or Yarrowia.

In some embodiments relating to production and purification of the recombinant amino acid sequences, the gene encoding the enzyme and/or regulatory protein is cloned into an expression vector such as the pET expression vectors from Novagen, transformed into a protease deficient strain of E. coli such as BL21 and expressed by induction with IPTG. The protein of interest may be tagged with an affinity tag to facilitate purification, e.g. hexahistidine, GST, calmodulin, TAP, AP, CAT, HA, FLAG, MBP etc. Coexpression of a bacterial chaperone such as dnaK, GroES/GroEL or SecY may help facilitate protein folding. See Green and Sambrook (2012).

Most fungi that make ergoline molecules contain the endogenous ergoline pathway genes in a single region of chromosomal DNA termed a cluster. Knowledge of this cluster can be used to locate the similar clusters in other species that are not known to make ergolines, but which may have some upstream pathway genes in common with the ergoline-producing fungi. The cluster sequence can also be used to identify potential ergoline clusters in newly discovered or newly sequenced species.

The enzymes from fungi known to produce ergolines, such as those in the family Claviceptacae, Trichocomaceae, Aspergillacea, Arthroderma can be modified to remain active in a heterologous host. The enzymes can also be isolated and modified from bacteria and plants that contain a homologous cluster of biosynthetic enzymes that contain, e.g., an indole prenyl transferase, N-methyltransferase and other functional enzymes of the ergoline pathway. Enzymes may be used alone or co-expressed to maximize flux through the ergoline pathway. Examples of plants that can be sourced for ergoline enzymes include morning glory plants Ipomoea vioaceae and Rivea corymbosa (Mexican ololiuqui), the Hawaiian baby woodrose (Argyrea nervosa), and the Hawaiian woodrose (Merremia tuberose).

Each candidate polypeptide (enzyme) is introduced into a host cell genetically modified to contain all necessary components for ergoline biosynthesis using standard yeast, fungal, or bacterial cell transformation techniques (Maniatis or Gietz, 1995). Cells are subjected to fermentation, under conditions that activate the promoter controlling the candidate polypeptide and broth subjected to HIPLC analysis.

TABLE 2
Summary of exemplary polynucleotide and polypeptide sequences

| | Nucleic Acid | Amino Acid |
|---|---|---|
| Shorthand | SEQ ID NOs | SEQ ID NOs |
| EasE | SEQ ID NOs: 1-11 | SEQ ID NOs: 168-178 |
| EasC | SEQ ID NOs: 12-19 | SEQ ID NOs: 179-186 |
| EasD | SEQ ID NOs: 20-29 | SEQ ID NOs: 187-196 |
| EasA | SEQ ID NOs: 30-38 | SEQ ID NOs: 197-205 |
| EasG | SEQ ID NOs: 39-47 | SEQ ID NOs: 206-214 |
| CloA | SEQ ID NOs: 48-53 | SEQ ID NOs: 215-220 |
| CYP | SEQ ID NOs: 54-58 | SEQ ID NOs: 221-225 |
| CPR | SEQ ID NOs: 59-71 | SEQ ID NOs: 226-238 |
| CYB | SEQ ID NOs: 72-79 | SEQ ID NOs: 239-246 |
| LPS | SEQ ID NOs: 80-89 | SEQ ID NOs: 247-256 |
| EasM | SEQ ID NOs: 90-94 | SEQ ID NOs: 257-261 |
| EasN | SEQ ID NOs: 95-99 | SEQ ID NOs: 262-266 |
| EasL | SEQ ID NOs: 100-105 | SEQ ID NOs: 267-272 |
| EasH | SEQ ID NOs: 106-114 | SEQ ID NOs: 273-281 |
| EasO | SEQ ID NOs: 115-122 | SEQ ID NOs: 282-289 |
| EasP | SEQ ID NOs: 123-130 | SEQ ID NOs: 290-297 |
| HAL | SEQ ID NOs: 131-134 | SEQ ID NOs: 298-301 |
| UGT | SEQ ID NOs: 135-138 | SEQ ID NOs: 302-305 |
| TrpS | SEQ ID NOs: 139 | SEQ ID NOs: 306 |
| Scaffold | SEQ ID NOs: 140 | SEQ ID NOs: 307 |
| affibody_tag | SEQ ID NOs: 141-142 | SEQ ID NOs: 308-309 |
| localization tags | SEQ ID NOs: 143-165 | SEQ ID NOs: 310-332 |
| co-folding tags | SEQ ID NOs: 166-167 | SEQ ID NOs: 333-334 |

In some aspects relating to sequences comprising amino acids (e.g., enzymes) and nucleotides (e.g., polynucleotides/genes), the disclosure provides sequence variants of the sequences disclosed herein. With regard to amino acid sequences, variants (e.g., substitution, deletion, addition) can be considered as “similar” to the original polypeptide or to have a certain “percent identity” to the original polypeptide, and include those polypeptides wherein one or more amino acid residues of a polypeptide are removed and replaced with alternative residues. In embodiments, the substitutions in amino acid sequences are conservative in nature, however, the disclosure embraces substitutions that are also non-conservative.

For amino acid sequences, sequence identity and/or similarity can be determined by using standard techniques known in the art such as, for example, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, using the default settings, or by inspection. Various alignment parameters can be set according to known methods (e.g., “Current Methods in Sequence Comparison and Analysis,” Macromolecule Sequencing and Synthesis, Selected Methods and Applications, pp 127-149 (1988), Alan R. Liss, Inc.). Additional useful algorithms include PILEUP, which can align multiple sequences from a group of related sequences using progressive, pairwise alignments; BLAST, including gapped BLAST, WU-BLAST-2 (see, e.g., Altschul et al., 1990, J. Mol. Biol. 215:403-410; Altschul et al., 1993, Nucl. Acids Res. 25:3389-3402; Altschul et al., 1997, Nucleic Acids Res. 25:3389-3402; Altschul et al., 1996, Methods in Enzymology 266:460-480; and Karin et al., 1993, Proc. Natl. Acad. Sci. U.S.A. 90:5873-5787).

Generally, the amino acid homology, similarity, or identity between sequences are at least 80%, including at least 85%, 90%, 91%, 92%, 93%, 94%, 95%, 96%, 97%, 98%, 99%, and from 99% to almost 100% identity. Similarly, the “percent (%) nucleic acid sequence identity” with respect to the nucleic acid sequences described herein refers to the percentage of nucleotide residues in a candidate sequence that are identical with the nucleotides disclosed herein, in the gene or coding sequence of the related polypeptide. Specific methods can include the default parameters of algorithms such as, for example, BLASTN (WU-BLAST-2).

### Recombinant Host Cells

In an aspect the disclosure provides recombinant cells that comprise the polynucleotides and polypeptides described herein. In embodiments, the host cells can comprise any type of cell that is adaptable to genetic manipulation and/or expression of foreign genes and proteins. In some embodiments, host cells can include any species of filamentous fungus, including but not limited to any species of Aspergillus, which may be optionally genetically altered to accumulate and/or produce precursor or intermediate ergoline molecules. In embodiments, host cells may also be any species of bacteria, including but not limited to Escherichia, Corynebacterium, Caulobacter, Pseudomonas, Streptomyces, Bacillus, or Lactobacillus. In some embodiments, host cell is a yeast cell capable of being genetically engineered can be utilized in these embodiments. Non-limiting examples of such yeast cells include species of Saccharomyces, Candida, Pichia, Schizosaccharomyces, Scheffersomyces, Blakeslea, Rhodotorula, or Yarrowia.

These cells can achieve gene expression controlled by inducible promoter systems, natural or induced mutagenesis, recombination, and/or shuffling of genes, pathways, and whole cells performed sequentially or in cycles; overexpression and/or deletion of single or multiple genes and reducing or eliminating parasitic side pathways that reduce precursor concentration.

The host cells of the recombinant organism may also be engineered to produce any or all precursor molecules necessary for the biosynthesis of ergolines and can comprise any of the disclosed polynucleotide sequences, vectors or expression cassettes that are capable of expressing the recombinant enzyme encoded therein.

Construction of modified host cells such as Saccharomyces cerevisiae strains expressing the enzymes and regulatory proteins provided herein is carried out via expression of a gene which encodes for the enzyme. The gene encoding the enzyme can be cloned into vectors with the proper regulatory elements for gene expression (e.g. promoter, terminator) and the derived plasmid can be confirmed by DNA sequencing. As an alternative to expression from an episomal plasmid, the gene encoding the enzyme may be inserted into the recombinant host genome. Integration may be achieved by a single or double cross-over insertion event of a plasmid, or by nuclease-based genome editing methods, as are known in the art e.g. CRISPR, TALEN and ZFR. Strains with the integrated gene can be screened by rescue of auxotrophy and genome sequencing. See, e.g., Green and Sambrook (2012).

As described herein, the recombinant cell may be any species of yeast, including but not limited to any species of Saccharomyces, Candida, Schizosaccharomyces, Yarrowia, etc., which have been genetically altered to produce ergoline molecules, including precursors, intermediates, and metabolites thereof. Some of the species of yeast include but are not limited to: Schizosaccharomyces cerevisiae, Schizosaccharomyces japonicus, Schizosaccharomyces pombe, Schizosaccharomyces cryophilus, Saccharomyces cerevisiae, Kluyveromyces lactis, Kluyveromyces dobzhanskii, and Yarrowia lipolytica. Additionally, genetically engineered host cells may be any species of filamentous fungus, including but not limited to any species of Aspergillus, which have been genetically altered to produce precursor molecules such as L-tryptophan, DMAT, and DMA-L-abrine molecules.

In various embodiments, the recombinant cell can comprise combinations of endogenously upregulated and/or codon optimized genes encoding for proteins which can improve: protein production, correct protein folding, protein secretion, proper protein intracellular localization, enzyme activity, correct post-translational modifications for protein features, mRNA stability, cell tolerance to stress, cellular metabolic activity, availability of cofactors for enzyme activity, glycolysis, fatty acid metabolism, feedstock conversion, amino acid biosynthesis, mevalonate pathway flux, coenzyme A (CoA) flux, acetyl-CoA production, fatty-acyl production, short chain alcohol (e.g. ethanol, butanol) tolerance, tolerance to oxidative stress (e.g. H2O2) from increased protein production, tolerance to oxidative stress from ergoline enzymatic pathway steps, titer of ergoline pathway precursors, intermediates, and compounds. In some related embodiments, the genes and proteins that may be expressed by the recombinant cell (with reference to exemplary Genbank protein accession numbers) can include one or more of:
rRNA processing proteins for ribosome biogenesis, such as BFR2 (QHB07760.1)
14-3-3 proteins which regulate vesicle and protein transport, such as BMH2 (CAA59275.1)
Transcription factors which upregulate the unfolded protein response, such as HAC1 (QHB08305.1)
Karyopherin overexpression to help the transport and translocation of hydrophobic and/or chimeric proteins, such as PSE1 (QHB11037.1)
Ribosomal proteins for maintaining translational productivity through cell stress, such as RPPO (QHB10483.1),
Oxidative stress protection, such as CCS1 (QHB10774.1), SOD1, (CAA89634.1), and/or SSA4, (KZV11871.1)
ER stress sensors which can upregulate protein folding chaperones and heat shock responses, such as IRE1 (QHB09072.1)
Proteins which can increase cellular glycosylation status, such asPSA1, (CAA98617.1)
Peroxide consuming proteins including catalase to protect from oxidative damage, such as CTT1, (CAA97090.1)
Protein folding chaperones and chaperonins, such as SSE1, (KZV07411.1), JEM1, (QHB09554.1), KAR2, (CAA89325.1), LHS1, (CAA81910.1), SCJ1, (CAA41529.1), SIL1, (QHB11576.1), GroEL (QEP09777.1), GeoES (QEP09776.1), and/or SSS1, (CAA98906.1)
Post-translational support, such as UBI4, (CAA97489.1)
Isomerases for proper protein folding, such as CPR5 (KZV12543.1) and/or CPR2 (KZV10787.1)
Heat shock response ER membrane proteins, including calnexin, such CNE1, (QHB06590.1)
Endoplasmic reticulum redox balance for proper protein folding including disulfide bond formation, such as ERO1, (QHB10609.1), PDI1, (KZV12810.1), and/or EUG1, (KZV12759.1),
Protein translocation and protein folding enhancers, such as SBH1, (KZV11852.1), and/or SEC61 (CAA44215.1)
Enhancers of protein trafficking, vesicle formation, tethering, and fusion between organelles and membranes, SEC31(CAA98772.1), SLY1, (CAA38221.1), BOS1, (CAA97636.1), BET1, (CAA86247.1), SEC22, (QHB10420.1), SED5, (CAA97549.1), COG7, (QHB08640.1), COY1, (QHB09825.1), INM1, (QHB10458.1), SEC1, (QHB07623.1), SEC4, (QHB08330.1), SNC1, (KZV13437.1), SNC2, (CAA89974.1), SSO1, (CAA97949.1), and/or SSO2, (KZV09033.1)
Secretory pathway processing enzymes, including glycosyltransferase, KEX2, (CAA96143.1), MNN1, (AAA53676.1), MNN2, (QHB06786.1), MNN9, (KZV07467.1), MNN10 (QHB07704.1), MNN11, (QHB09454.1), and/or OCH1, (CAA96740.1)
Cell wall related proteins for stress tolerance and cell wall stability, such as CCW12 (KZV09357.1), CWP2, (CAA81937.1), YPS1, (KZV09366.1), MKC7, (KZV12382.1), and/or SED1 (BAI99734.1)
Flavin cofactor synthesis and recycling proteins for FAD+/FADH use by enzymes expressed in a modified host, such as FAD1 (CAA98604.1) or FMN1 (NM_001180544.1)
Nicotinamide cofactor synthesis and recycling proteins for NAD(P)+/NAD(P)H use by enzymes expressed in a modified host, such as UTR1 (CAA89577.1), POS5 (CAA97900.1),
Upregulation of the pentose phosphate pathway (PPP) such as increased expression of pentose phosphate genes encoding for enzymes in the PPP, such as GND1 (KZV10926.1) and/or ZWF1 (CAA96146.1)
Enzymes to increase acetyl-CoA pool including alcohol and aldehyde dehydrogenases and acetyl-CoA synthetases such as ALD6 (QEP09776.1), ADH2 (QEP09776.1), and/or ACS (AAL23099.1)
Enzymes to upregulate heme biosynthesis, such as delta-aminolevulinic acid dehydratase (Hem2) (CAA96742.1), porphobilinogen deaminase (Hem3) (CAA98783.1), uroporphyrinogen decarboxylase (Hem12) (QHB07515.1), oxygen-dependent coproporphyrinogen III oxidase (Hem13) (QHB07512.1), and/or ferrochelatase (Hem15) (CAA99385.1).
Transporters to increase metal ion import, such as FET3 (QHB10789.1), FTR1(QHB08222.1), and/or FET4(QHB11048.1)
Metal ion oxidoreductases to convert metals into usable forms, such as the ferric and copper reductases, FRE1 (QHB10365.1) and/or FRE3(KZV08274.1)

In various embodiments, the recombinant cell comprising endogenous genes encoding proteins for an improved effect(s) for the expression of an ergoline may be adapted or manipulated (i.e., genetically modified) in a way that modifies endogenous gene expression. Non-limiting examples include swapping an endogenous promoter for the gene of interest with a stronger promoter (constitutive or inducible); codon optimized gene sequence encoding proteins (e.g., genetic integration, episomal plasmids, or artificial chromosomes of such genes in an expression cassette (e.g. promoter, coding region, terminator)) any of which can be accomplished using methods known in the art.

The recombinant cell can contain combinations of modifications to host genes where genes or combinations thereof have downregulated or no (i.e. knocked out) expression, and/or silenced message, which can improve: protein production, correct protein folding, protein secretion, proper protein intracellular localization, enzyme activity, correct post-translational modifications for protein features, mRNA stability, cell tolerance to stress, cellular metabolic activity, availability of cofactors for enzyme activity, glycolysis, fatty acid metabolism, feedstock conversion, amino acid biosynthesis, mevalonate pathway flux, coenzyme A (CoA) flux, acetyl-CoA production, fatty-acyl production, short chain alcohol (e.g. ethanol, butanol) tolerance, tolerance to oxidative stress (e.g. H2O2) from increased protein production, tolerance to oxidative stress from ergoline enzymatic pathway steps, titer of ergoline pathway precursors, intermediates, and compounds.

Such genes (with examples of Genbank nucleotide accession numbers) include combinations thereof:
Transcription factors such as ROX1 (NM_001184162.1)
Heme depletors, including oxygensases, such as HMX1 (NM_001182092)
Proteases, such as PEP4 (NM_001183968.1), PRC1 (NM_001182806.1), PRB1 (NM_001178875.1)
Ubiquitin ligases, such as BUL1 (NM_001182782.1) and/or BUL2(NM_001182473.1)
O-acetyltransferases, such as ATF1 (NM_001183797) and/or ATF2(NM_001181306.1)
Dehydrogenases and reductases, such as ARI1 (NM_001181022), ADH6 (NM_001182831.3), OYE2 (NM_001179310), and/or OYE3 (NM_001183985.1)
Genes encoding for proteins involved in amino acid metabolism, such as BNA2 (NM_001181736.3), ARO10 (KU050081.1), PDC6 (NM_001181216.3).

### Biosynthetic Methods

In some aspects, the disclosure provide a biosynthetic method for producing an ergoline, or a precursor or metabolite thereof, comprising: (i) generating a recombinant host cell; (ii) growing the recombinant host cell under conditions effective to produce an ergoline, or precursor thereof; and (iii) isolate the ergoline or precursor thereof from the recombinant host cell. and thereby yielding a recombinant host organism; (ii) expressing engineered ergoline biosynthesis genes and enzymes in the recombinant host organism; (iii) producing or synthesizing ergolines in the recombinant host organism; (iv) fermenting the recombinant host organism; and (v) isolating the ergolines from the recombinant host organism. Endogenous pathways of the recombinant host can be modified by the systems and methods herein to produce high purity ergolines.

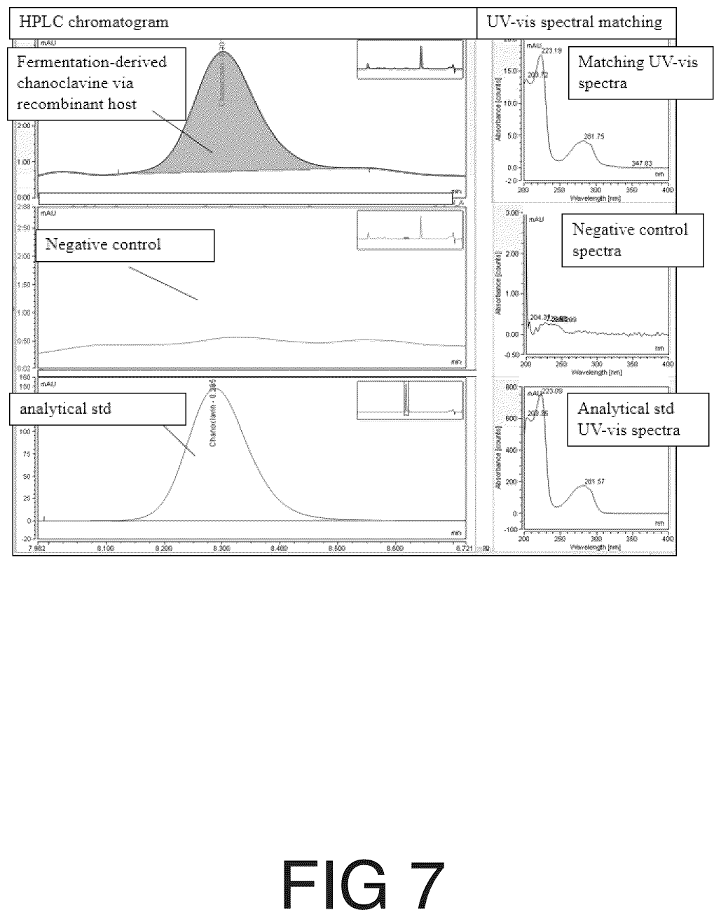

In various embodiments of the biosynthetic methods, the nucleic acid encoding the enzymes and/or regulatory proteins are introduced into a host cell using standard cell (e.g., yeast) transformation techniques (Green and Sambrook, 2012). Cells are subjected to fermentation under conditions that activate the promoter controlling the synthesis of the enzyme and/or regulatory protein. The broth may be subsequently subjected to HPLC analysis to determine the presence or yield of the desired ergoline pathway product, as in FIG. 7.

In various embodiments of the biosynthetic methods, the host cells are provided with various feedstocks to drive production of the desired ergolines, e.g., glucose, fructose, sucrose, galactose, raffinose, maltose, ethanol, xylose, fatty acids, glycerol, acetate, molasses, malt syrup, corn steep liquor, dairy, flour, protein powder, olive mill waste, fish waste, etc. as is known in the art.

### Biosynthetic Pathway to Generate Ergolines

Biosynthetic pathways for production of ergolines, such as lysergic acid, and related downstream compounds from Claviceps and related fungi have been described. Some fungi can produce early pathway intermediates derived from tryptophan, but lack the enzymes required for production of downstream ergolines which are only found in fungi that make psychoactive compounds. For example, Aspergillus can synthesize non-psychoactive ergoline molecules such as cycloclavine or fumigaclavine. Such species can be utilized as a source of potential enzymes for heterologous expression or co-expression of the polynucleotide and protein sequences disclosed herein.

Table 3 provides a general description and overview of sequences that are relevant to ergoline biosynthesis.

TABLE 3
Sequences and functionality in the ergoline biosynthetic pathway

| Shorthand | General description | Embodiments of biosynthetic reaction(s) |
|---|---|---|
| EasE | Oxidase | EasE together with EasC can generate chanoclavine |
| | | from N-Me DMAT |
| EasC | Catalase | (above) |
| EasD | Chanoclavine-I | EasD can oxidize chanoclavine to chanoclavine |
| | dehydrogenase | aldehyde; can be combined with EasE, EasC and/or |
| | | either or both EasA and EasG |
| EasA | Chanoclavine | EasA reductase can enhance agroclavine and |
| | reductase | festuclavine production with either or both of EasG |
| | | and EasD |
| EasG | Dehydrogenase | EasG can convert chanoclavine aldehyde to |
| | | agroclavine or festuclavine with either or both of |
| | | EasD and EasA |
| CloA | Oxidase | CloA can oxidize agroclavine to paspalic acid |
| CYP | Cytochrome P450 | Exemplary P450 CYP3A4 can oxidize LSD to form |
| | | nor-LSD |
| CPR | Cytochrome reductase | CPR can assist with electron transfer for P450 enzyme |
| | | function |
| CYB | Cytochrome B | CYB involved in electron transport and can assist |
| | | P450 enzyme function |
| LPS | Lysergyl peptide | LPS can carry out non ribosomal peptide synthesis on |
| | synthetase | lysergic acid to generate peptide-type ergot alkaloids |
| EasM | Festuclavine oxidase | EasM can add a hydroxyl group to festuclavine |
| EasN | Acetylase | EasN can acetylate fumigaclavine B to generate |
| | | fumigaclavine A |
| EasL | Fumigaclavine | EasL can prenylate fumigaclavine A to generate |
| | prenyltransferase | fumigaclavine C |
| EasH | Dioxygenase | EasH and EasO can generate lysergic acid amides |
| | | from peptide-type ergot alkaloids |
| EasO | Monooxygenase | (above) |
| EasP | Hydrolase | EasP can act as a hydrolase and release a lysergic acid |
| | | amide from LPS |
| HAL | Halogenases | Can halogenate (e.g., chlorinate, fluorinate, |
| | | brominate) the indole ring of tryptophan |
| UGT | Glycosyltransferase | Can glucorinate LSA, or generate ergoline glycosides |
| TrpS | Tryptophan synthetase | Can generate tryptophan from indole (or modified |
| | | indoles) and L-serine |
| Scaffold | Fused affibody Z | Can localize several enzymes for processive |
| | domains for | modification of substrate |
| | localization of other | |
| | tagged enzymes | |
| Affibody | Affibody tags that | A localization tag that can bind a localization scaffold |
| tag | enable localization of | |
| | enzymes to the | |
| | scaffold | |
| localization | Localization tags e.g., | A secretion tag that can target an enzyme for secretion |
| tags | from S. cerevisaie, E | outside the cell |
| | coli, Pichia | |
| co-folding | Chaperone/Co- | A fusion peptide or polypeptide which can promote |
| tags | folding peptide | proper protein folding and yield of functional enzymes |
| | sequences, e.g. MBP | |

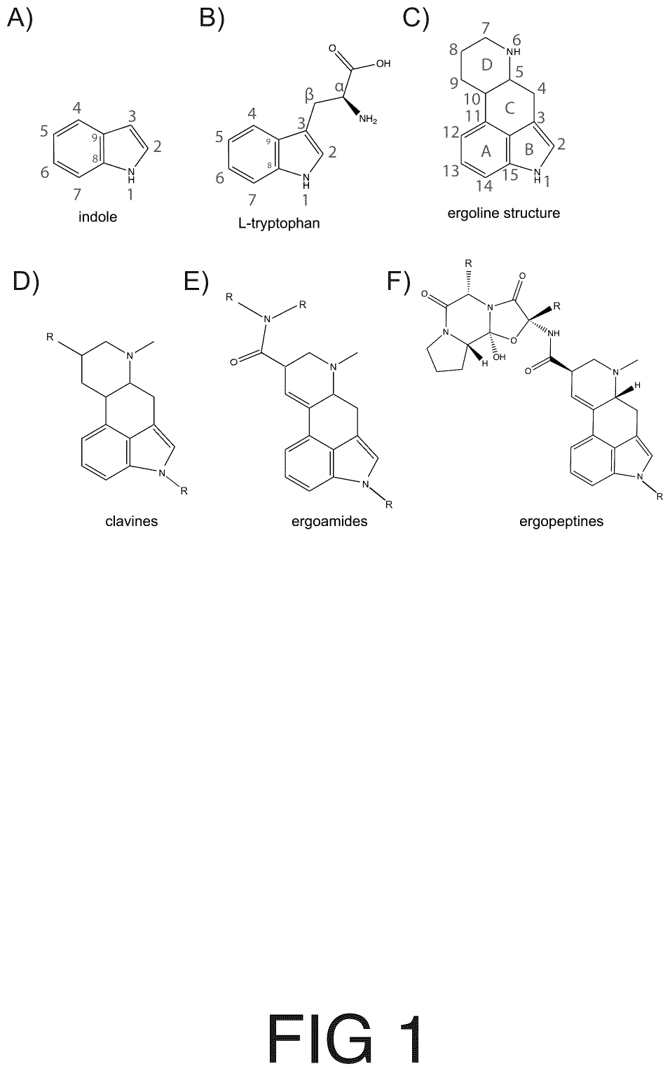

FIG. 1 describes several of the ergoline structures that fall within the scope of the disclosure. FIG. 1A illustrates an indole ring structure. In an ergoline synthetic pathway, additional modifications and ring formations at the 3 and 4 position of the indole lead to higher order structures, such as the amino acid tryptophan (FIG. 1), which can build to the generalized ergoline structure depicted in FIG. 1C. Representative subgroups of ergolines include the clavines having a general structure as depicted in FIG. 1D, the ergoamides having a general structure as depicted in FIG. 1E, and the ergopeptines having a general structure as depicted in panel 1F. Various substituent groups can be incorporated on the ergoline ring structure by various methods, including by biosynthesis comprising one or more recombinant sequences in accordance with the aspects and embodiments described herein, that can be incorporated into a recombinant host cell.

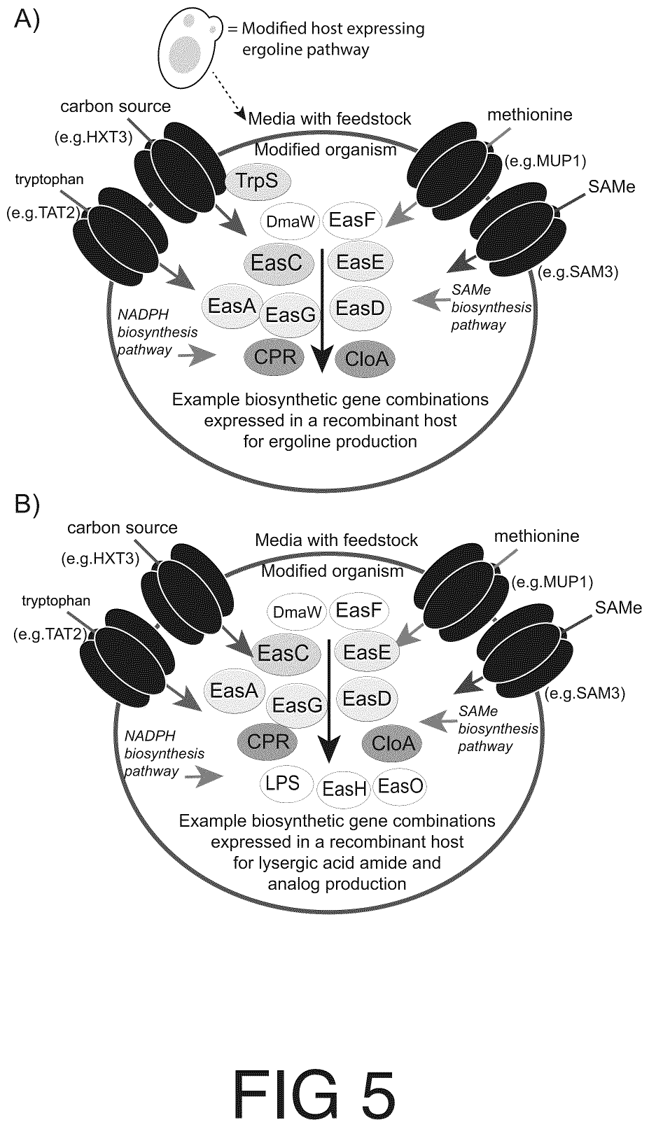

A recombinant host expressing the pathway for the ergoline biosynthesis pathway may contain any one, combinations, or all of the polynucleotide sequences and amino acid sequences described herein. For example, a recombinant biosynthetic pathway may be constructed to yield lysergic acid-type ergolines, in addition to genes encoding transporters of exogenous precursors such as L-tryptophan (FIG. 5 Panel A). Expressing additional genes in a recombinant host can yield peptide-type ergots and lysergic acid amides (FIG. 5 Panel B). A recombinant host expressing the ergoline biosynthesis pathway can also be used to further generate crude or purified lysate, and/or crude or purified enzymes which contain active ergoline biosynthesis enzymes and/or ergoline pathway products (e.g., precursors and metabolites).

A recombinant cell comprising an ergoline biosynthetic pathway is obtained by combining one or more EAS enzyme for any one or combination of steps in the biosynthetic process, and which can be derived from EAS enzymes or related amino acid sequences different species. In addition, the pathway can be optimized by analyzing buildup of intermediates to determine whether certain synthetic steps serve to limit the efficiency of the method. An optimized biosynthetic pathway typically comprises an enzyme that functions efficiently, or a combination of enzymes that function as a unit (i.e., have similar activity, reaction requirements, and efficiently translated by the host cell).

Generating Chanoclavines from Tryptophans

In embodiments of this aspect, the upstream ergoline biosynthetic pathway can begin with the amino acid L-tryptophan generated by any method known in the art. For example tryptophan can be biosynthetically created de novo from the shikimate pathway, or from the combination of precursor molecules, indole and serine. Alternatively tryptophan can be provided from an exogenous source (e.g., feedstock, serum, hydrosylates, etc.) comprising tryptophan.

Under typical growth conditions, cells tightly regulate de novotryptophan production. Accordingly, in accordance with some embodiments of the disclosure, tryptophan accumulation in a recombinant host cell can be increased by one or more of: (a) overexpressing feedback-resistant versions of tryptophan-synthesizing enzymes; (b) knocking out and/or inhibiting expression of off-pathway tryptophan-consuming genes and enzymes; and/or (c) overexpressing a recombinant L-tryptophan transporter for exogenous tryptophan import (see also U.S. Patent Publication 2021/0147888 and International publication WO 2021/248087). In some embodiments the biosynthesis can comprise (over)expression of enzymes TRP1, TRP2, TRP3, TRP4 and/or TRP5 which can increase intracellular tryptophan and maintain or direct tryptophan flux through the ergoline pathway. In some embodiments the biosynthesis can comprise (over)expression of one or more genes useful for tryptophan biosynthesis, such as ARO1, ARO4, AROL, SER1, SER3, SER33, and/or SER2, and including feedback resistance mutant versions thereof, which increase shikimate pathway flux and/or supply serine.

In some embodiments, tryptophan for ergoline biosynthesis is generated by tryptophan synthetases (TRPS) which combine indole and serine to form tryptophan. The indole precursors for this reaction can be from exogenous sources or generated through the metabolism of indol-3-glycerol phosphate.

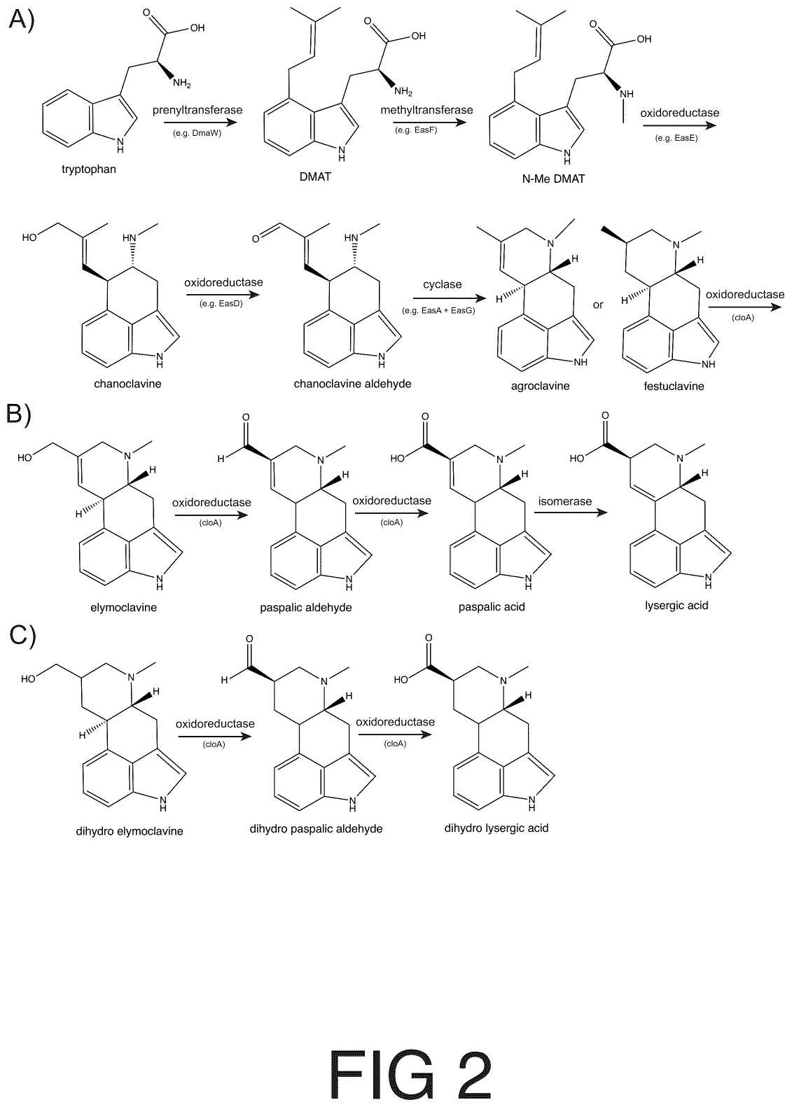

As a precursor for the ergoline pathway, L-tryptophan is first prenylated and then N-methylated to form dimethylallyl-L-abrine (Me-DMAT or DMA-abrine). A third ring off the two-ring indole structure in the ergoline skeleton is formed by cyclization of DMA-abrine to yield chanoclavine (FIG. 2, panel A). Relevant enzymes that are known in the art include dmaW and EasF.

This is a multistep reaction catalyzed by the enzyme pair of EASE, a berberine bridge enzyme (BBE)-like chanoclavine synthase, that is a FAD+ dependent enzyme, in partnership with a catalase-like EASC enzyme. Both enzymes are required for formation of the ring. Enzyme pairs may be sourced and modified from fungi known to produce ergolines, such as those in the family Claviceptacae, Trichocomaceae, Aspergillacea, and/or Arthroderma. They may also be sourced from bacteria and plants that contain a homologous cluster of biosynthetic enzymes that contains pairing of BBE-like FAD dependent oxidoreductase and catalase like enzyme. Enzymes may be used alone or co-expressed with other oxidases to maximize flux through the third step of the pathway.

Additionally, to facilitate this multistep cyclization mechanism, enzymes for a separate fungal plant or bacterial biosynthetic cluster may be necessary, such as epoxidases and oxidases.

Other strategies to maximize flux through the third step of the pathway include optimization of the N-terminal sequences of the oxidases. Most chanoclavine synthases contain an N-terminal secretion sequence. Cleavage of this sequence will help with expression of an active form of the protein in a heterologous host. Similarly, most EASC catalases contain a peroxisome localization signal (e.g. PTS1), which can be removed from the gene encoding the EASC enzymes by genetic engineering techniques known by those skilled in the art.

The N-terminal sequence may also be removed and replaced with chaperone-like sequences intended to facilitate folding such as the sequences disclosed herein.

The chanoclavine synthase may also be fused to its EASC partner after removal of the N-terminal secretory sequence.

The N-terminal secretory sequences may also be removed but replaced with a sequence that targets the enzyme to an intracellular organelle, such as the endoplasmic reticulum (ER), vacuole, peroxisome, mitochondria, or the Golgi-apparatus.

Protein folding to enhance enzyme activity may also be promoted by running the fermentation reactions at temperatures other than the standard 30° C., for example 15-40° C. Additional genes may be overexpressed to facilitate protein folding.

If chanoclavine biosynthesis-related protein sequences contain targeting sequences that direct the protein to other organelles, e.g. nucleus, mitochondria, peroxisome, vacuole, these sequences can be removed to ensure the protein is cytosolic and thus has access to its substrate.

Other strategies to increase the flux through the third step of the pathway include changing the location of the endogenous catalases of the yeast cell by adding or subtracting localization signals.

The EASE oxidoreductase depends on FAD+ as a cofactor, so increasing the intracellular supply of FAD+ may help maximize its activity. To this end, the FAD+ precursor riboflavin may be added to the culture medium. In addition, or alternatively, upregulating riboflavin biosynthesis genes may also be beneficial. Each molecule of chanoclavine generated consumes a molecule of oxidized FAD+ and reduces it to FADH₂. As such, introducing cellular mechanisms effective for regenerating the oxidized form of the cofactor will facilitate further catalytic cycles.

Other additions to the media can support chanoclavine biosynthesis. Examples include additions of extra vitamin mixtures, which can include choline chloride, niacin, pyridoxine hydrochloride, riboflavin, calcium pantothenate, para-aminobenzoic acid (PABA), thiamine HCL, biotin, cyanocobalamin, and/or folic acid, and mineral mixes, which can include calcium chloride dihydrate, ferrous sulfate heptahydrate, manganese (II) sulfate monohydrate, copper sulfate pentahydrate, zinc sulfate heptahydrate, magnesium chloride, and solutes, such as glycerol, up to 10% v/v. Since these are oxidoreductase reactions, they may be stimulated by changing the redox potential of the culture. This can be accomplished by addition of oxidants such as H₂O₂, sulfuric acid (H₂SO₄), nitric acid (HNO₃), potassium permanganate (KMnO₄), and/or Fenton's reagent, antioxidants such as ascorbic acid, butylated hydroxyanisole (BHA), and/or butylated hydroxytoluene (BHT), or reductants such as 2-Mercaptoethanol (B-ME), dithiothreitol (DTT), glutathione, cysteine hydrochloride, and/or tris(2-carboxyethyl)phosphine (TCEP).

Co-expression of multiple different EASE enzymes from multiple organisms with EASC may help increase effectiveness of the enzyme and conversely, co-expression of multiple different EASC enzymes in the presence of EASE. One or more particular stoichiometries of the EASE:EASC enzymes may also be beneficial to generating chanoclavine, and can be controlled by methods known in the art. For example, increasing the expression or activity of EASC relative to EASE to support higher levels of chanoclavine biosynthesis can include, for example, relative expression and/or activity levels that range from 1:1 to about 1:10 EASE to EASC (i.e., 1:1, 1:2, 1:3, 1:4, 1:5, 1:6, 1:7, 1:8, 1:9, or about 1:10). In some embodiments the ratio of expression or activity of EASE:EASC is from at least about 1:3 to about 1:10.

The EASE and EASC pairs may be arranged as a single gene by the use of linker sequences fusing the pair, as mentioned above, of fusion by linker sequences to express the pair as a single polypeptide or with a protease sequence, such as a 2A self-cleaving peptide, to separate the respective enzymes post-translation. In embodiments that comprise an increase in expression or activity of one or more enzymes relative to another enzyme, the expression or activity can be controlled by methods generally known in the art such as, for example, including increased copy numbers of the gene to be expressed in excess, modifying the promoters that control expression of the one or more genes to either increase or decrease expression levels, and the like.

Generating Agroclavine or Festuclavine from Chanoclavine

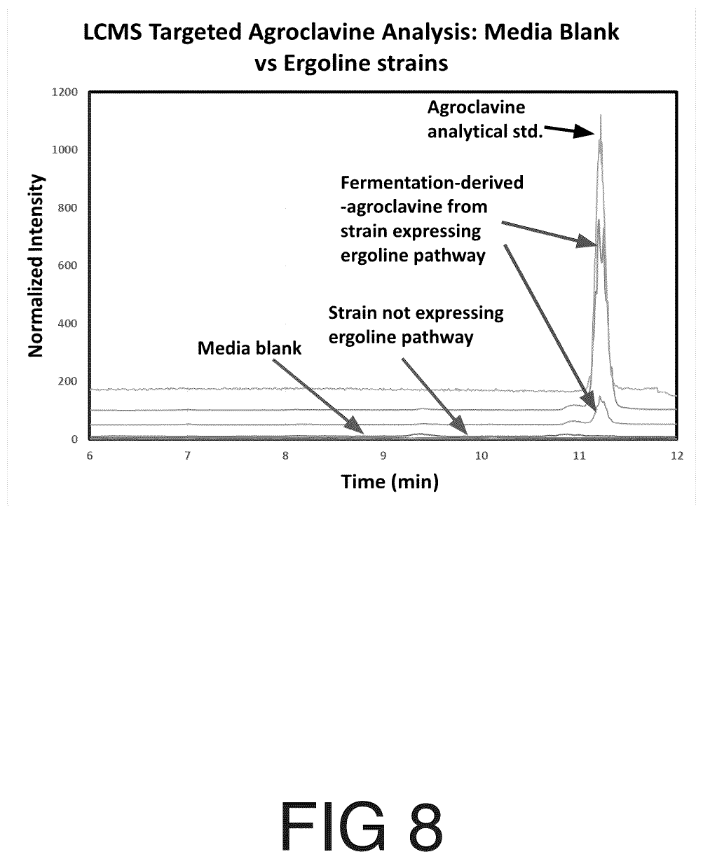

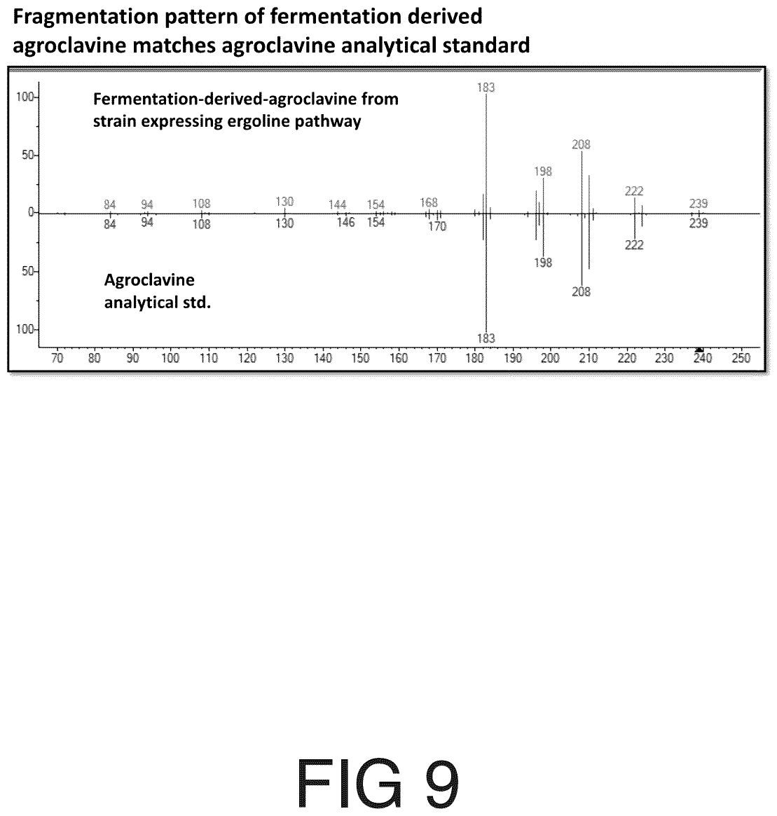

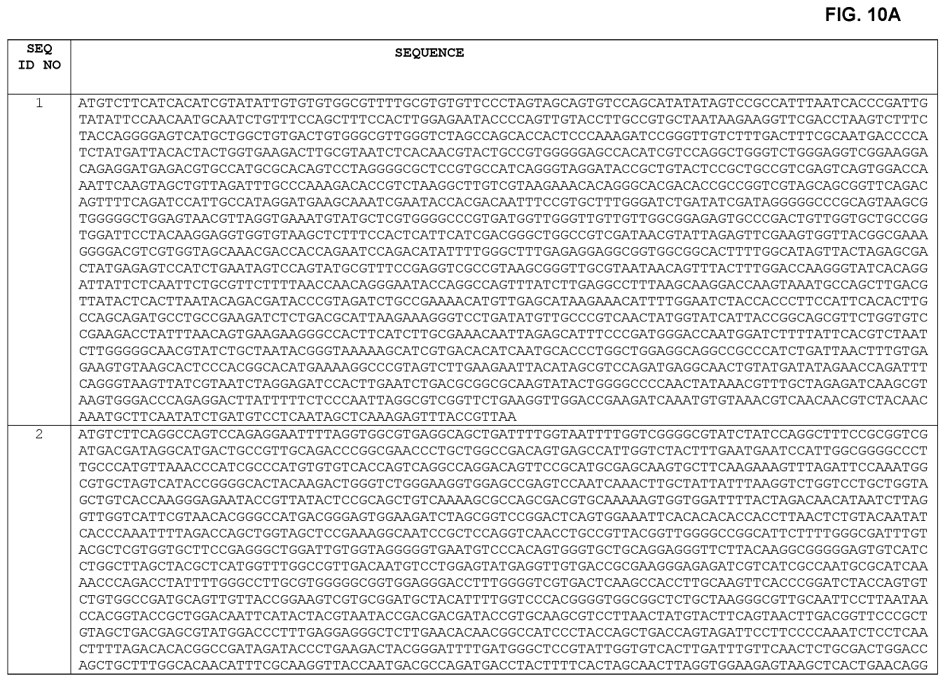

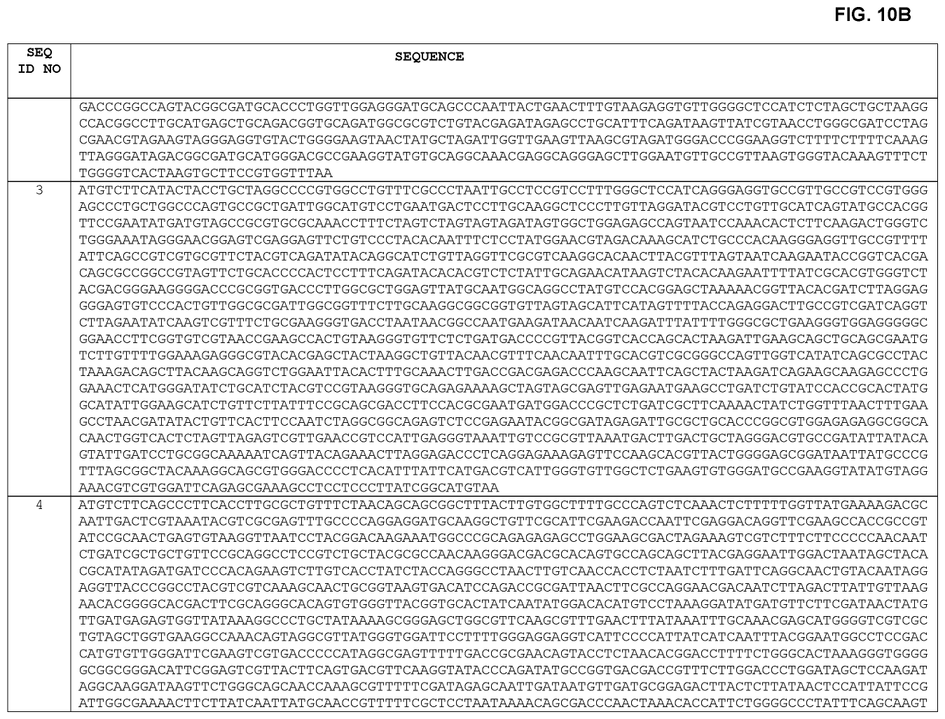

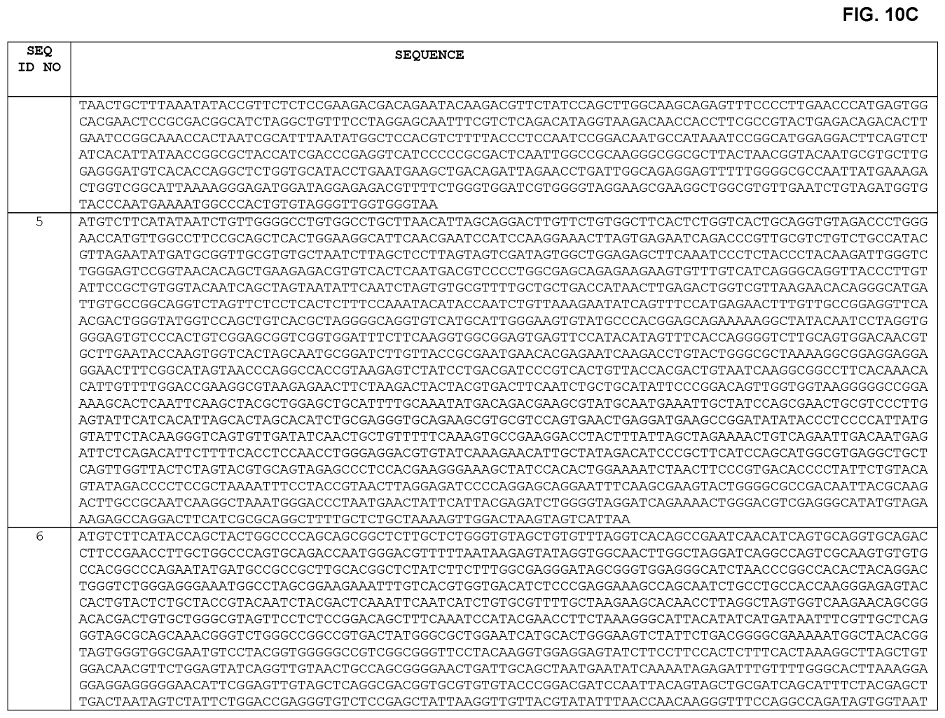

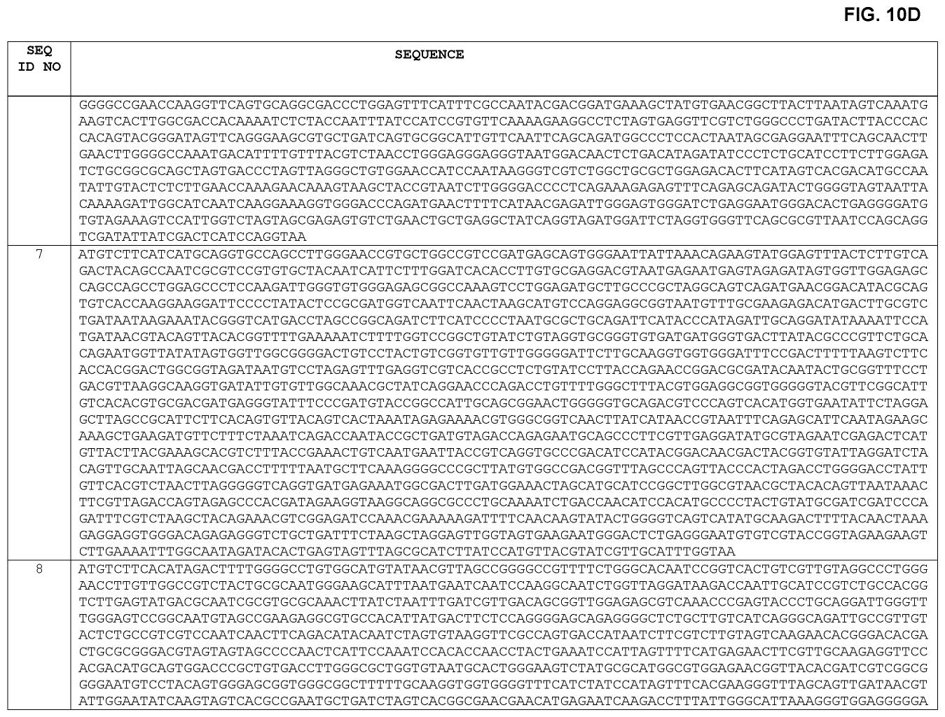

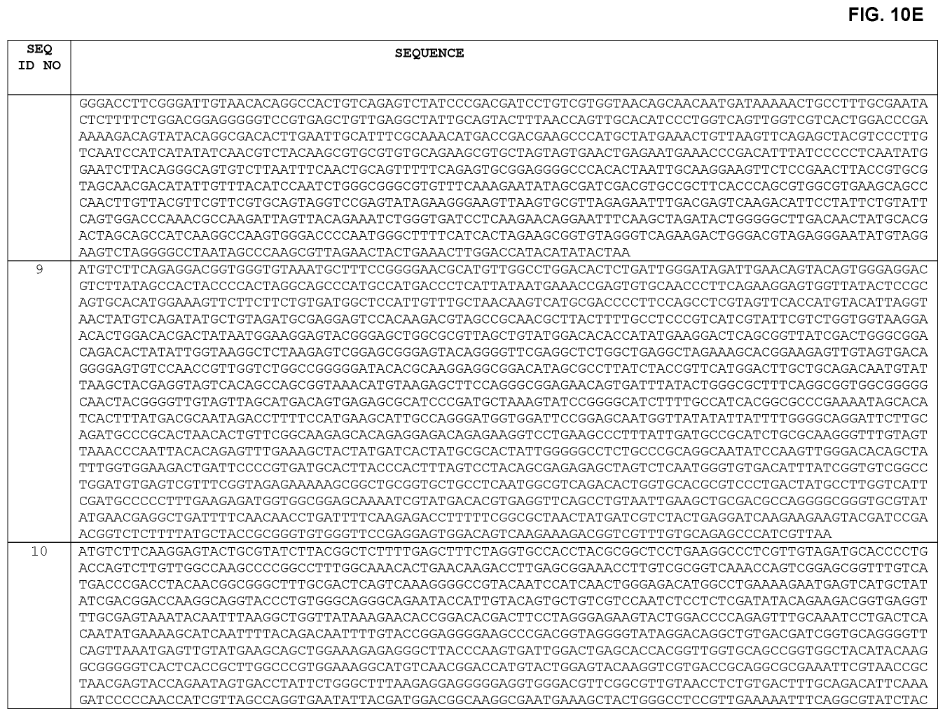

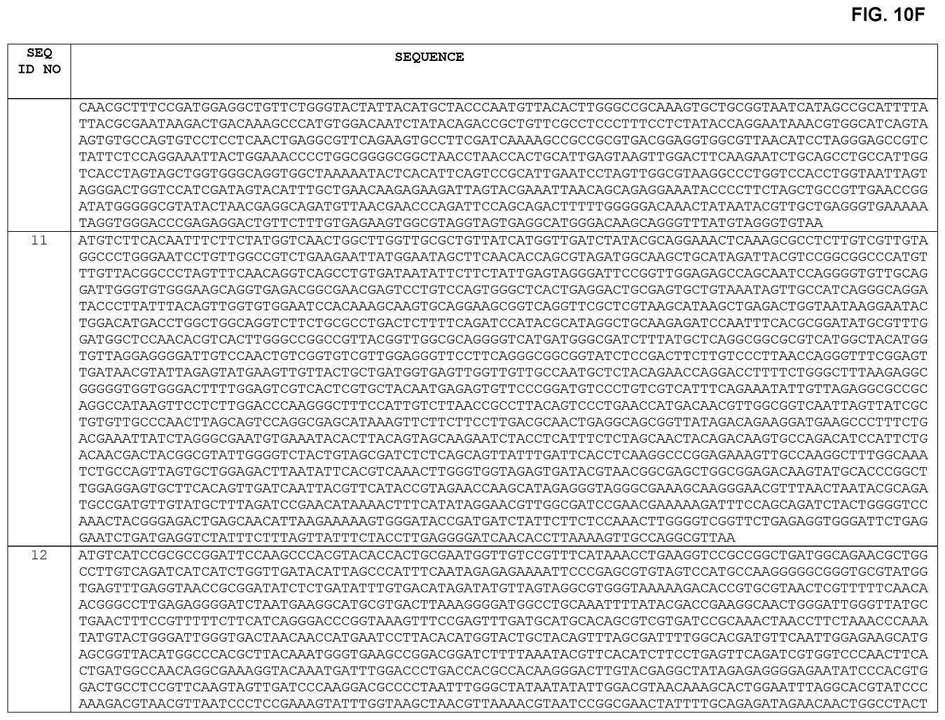

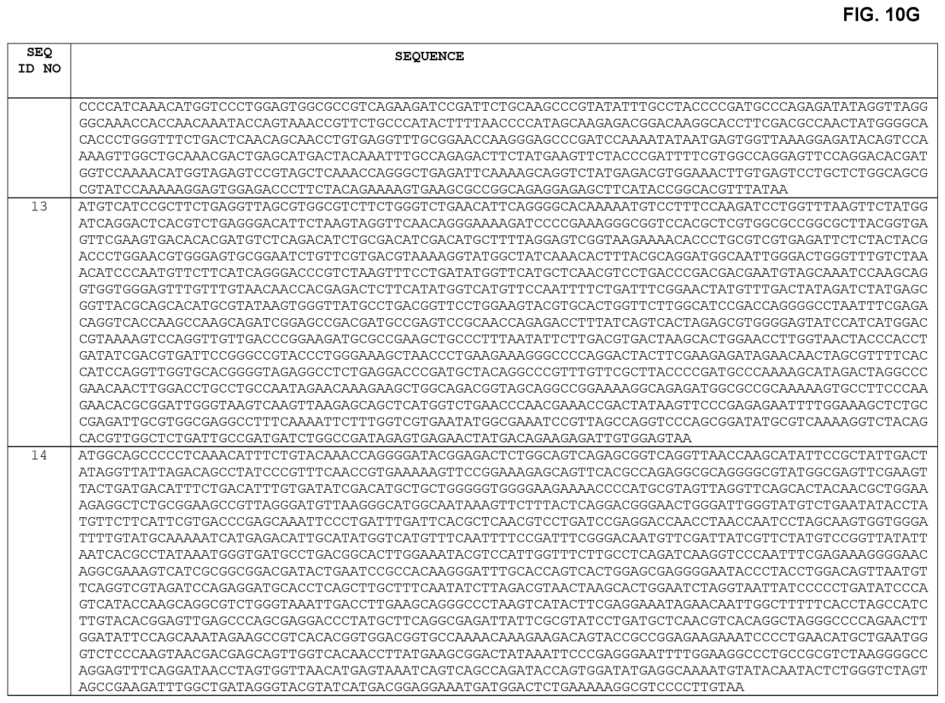

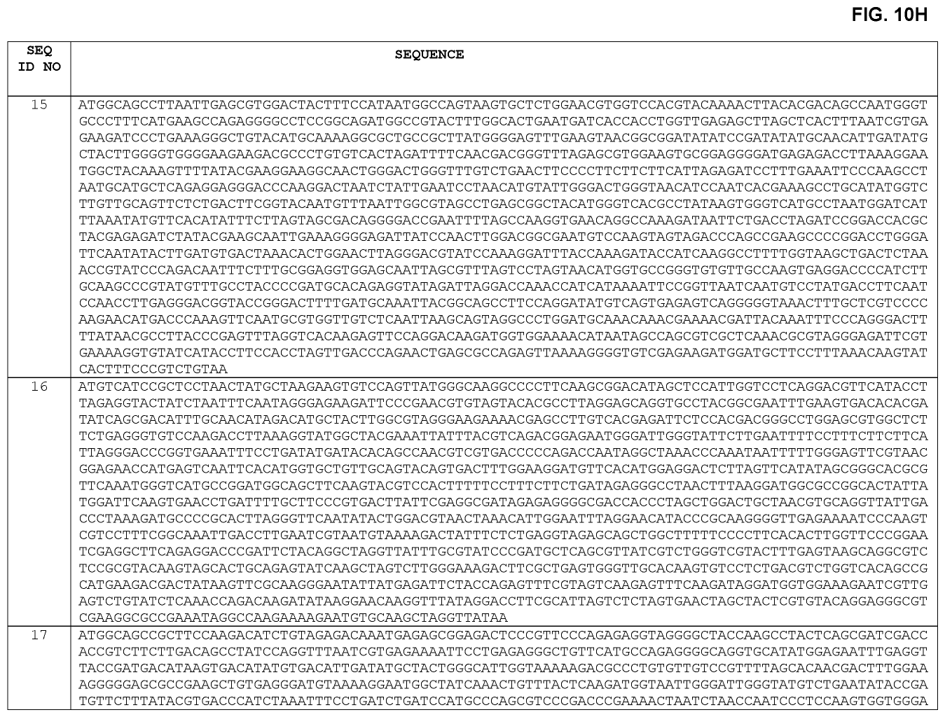

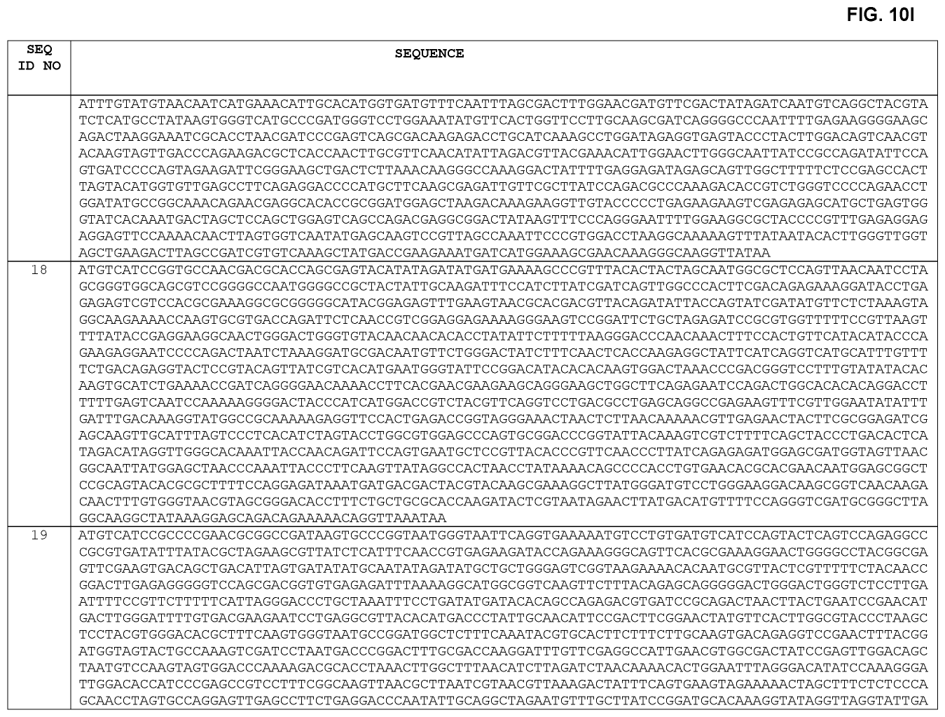

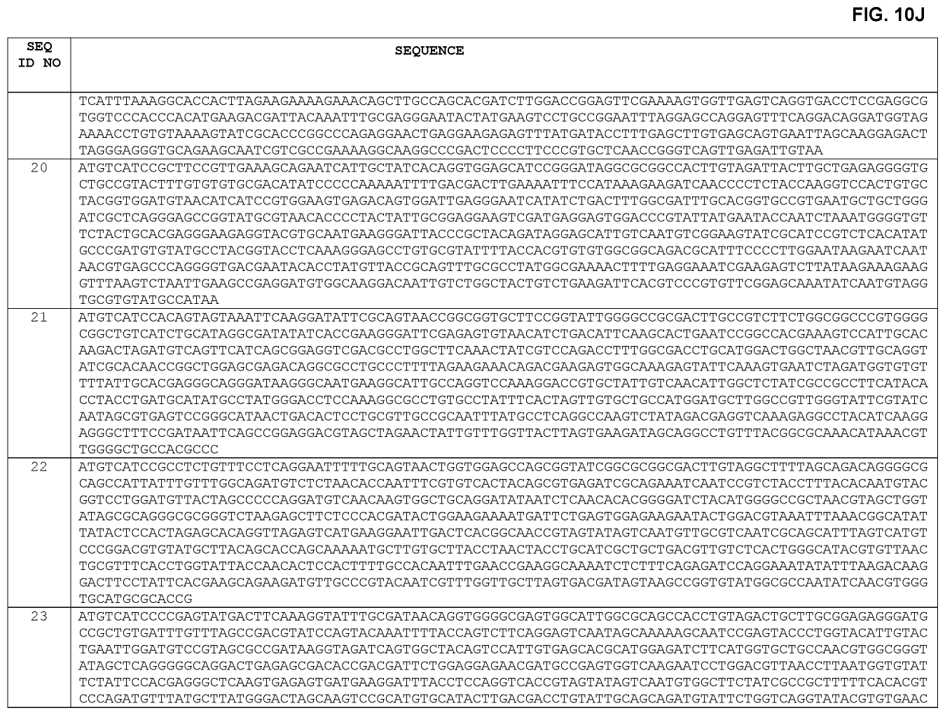

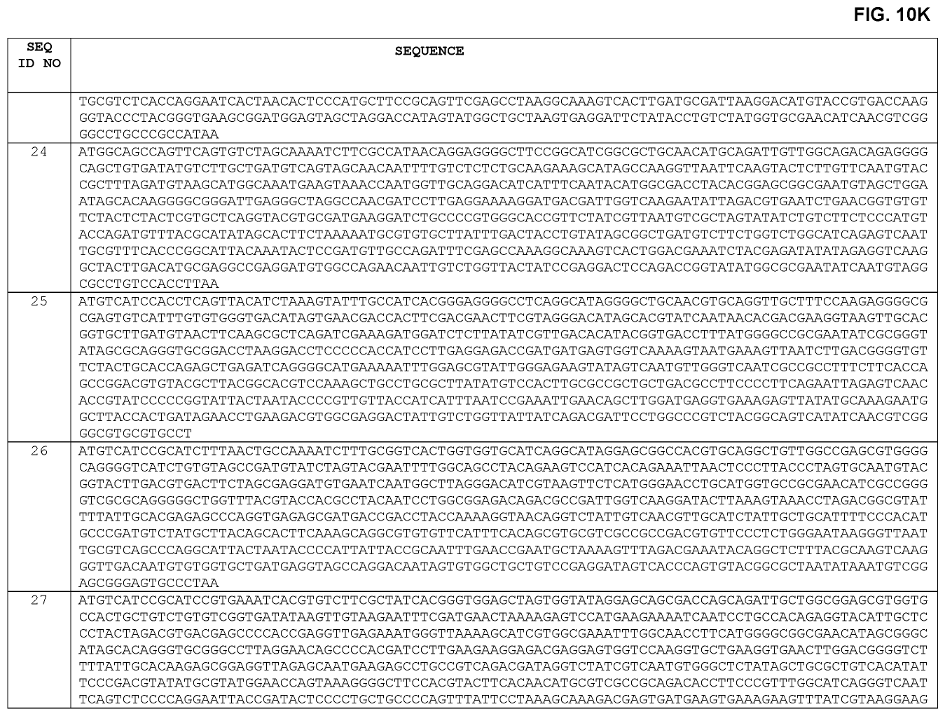

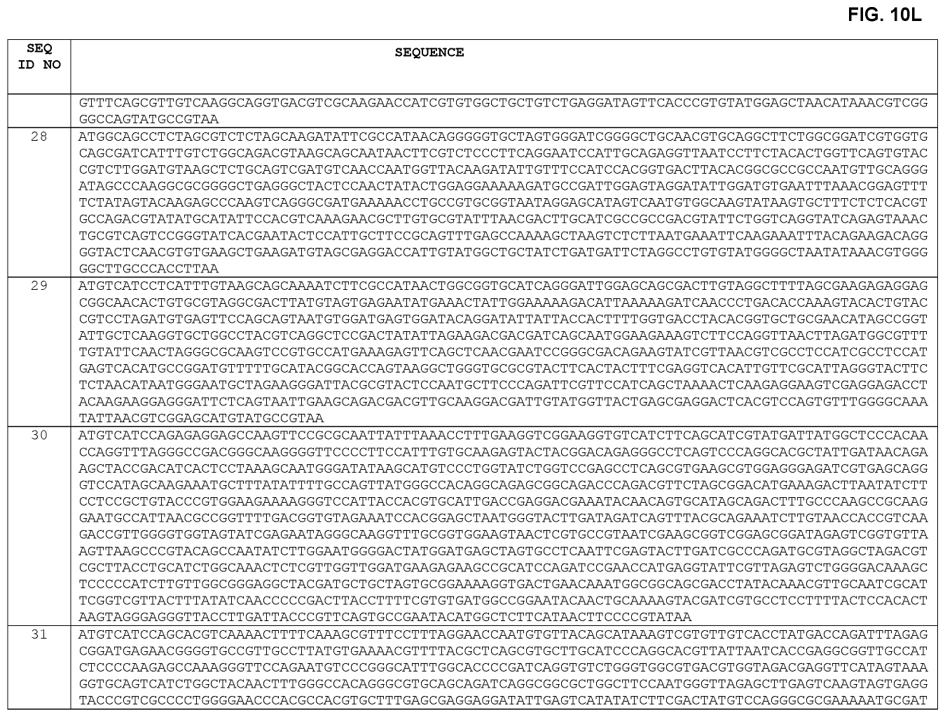

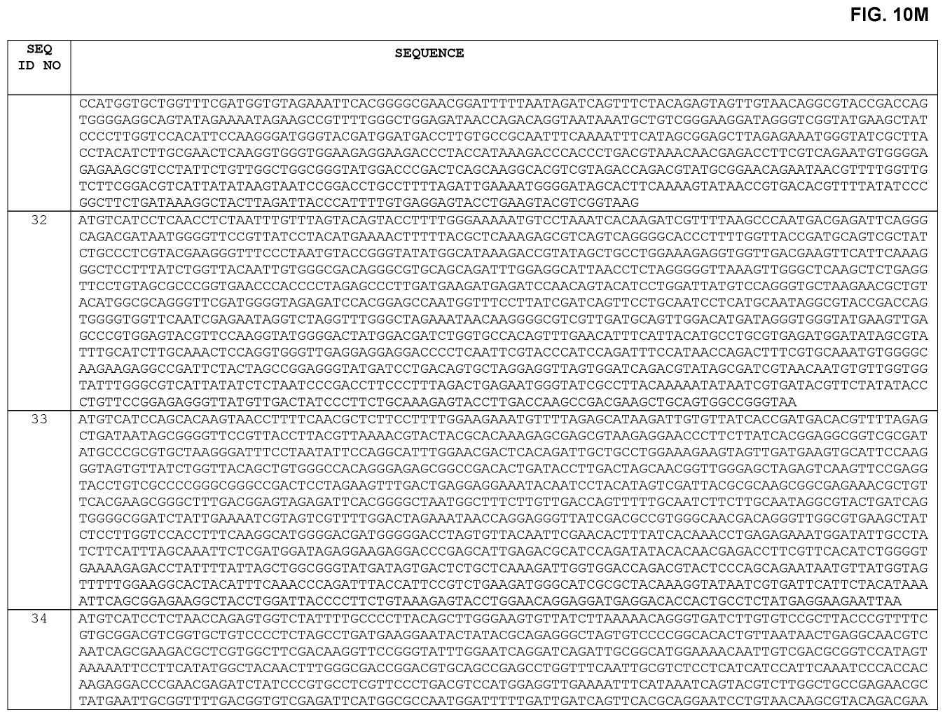

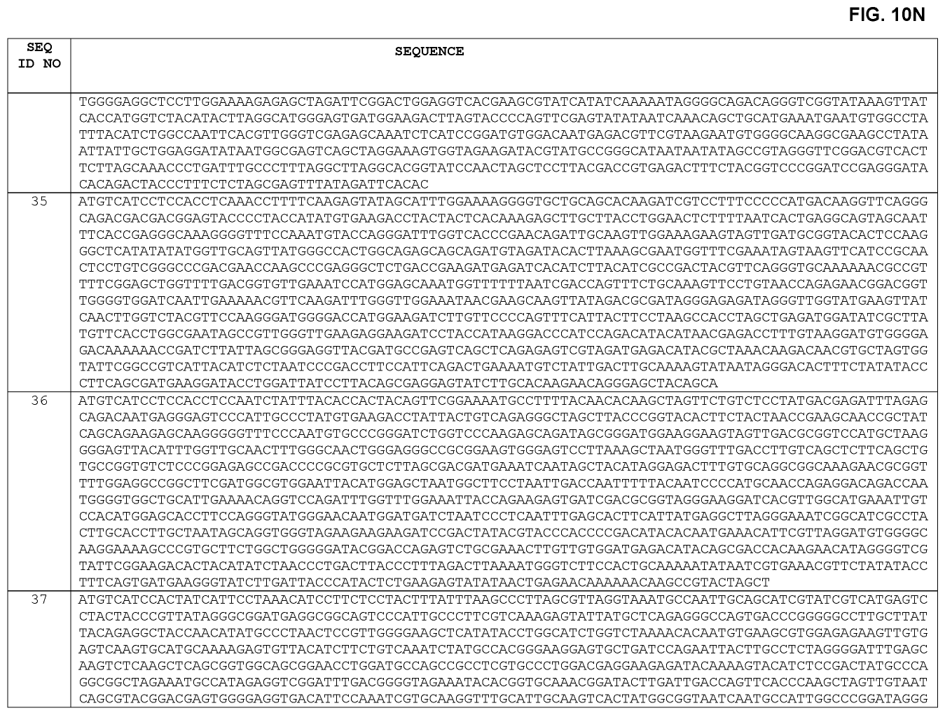

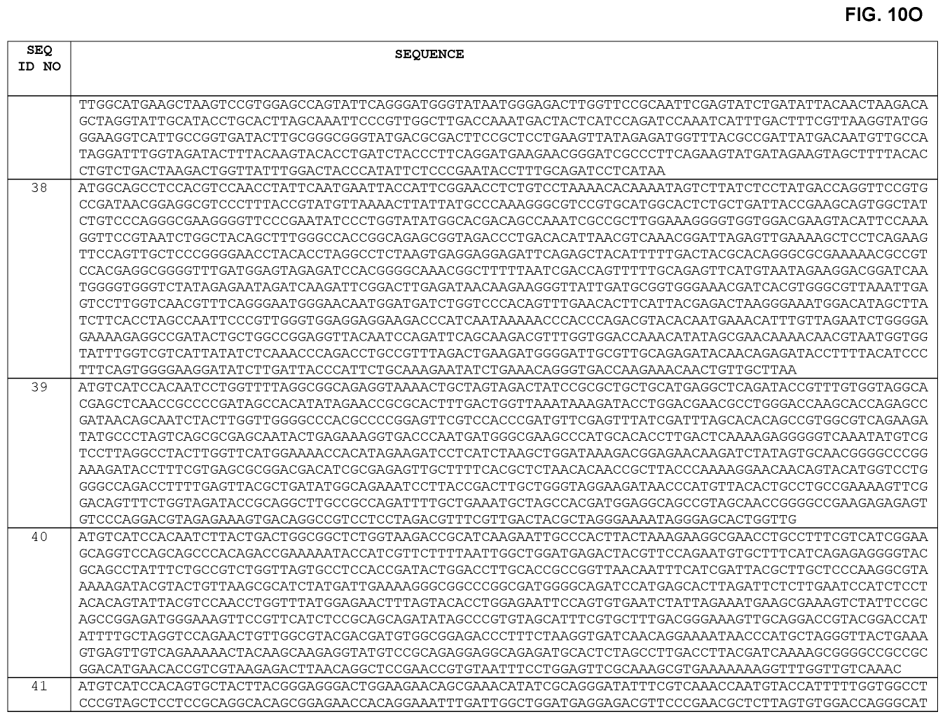

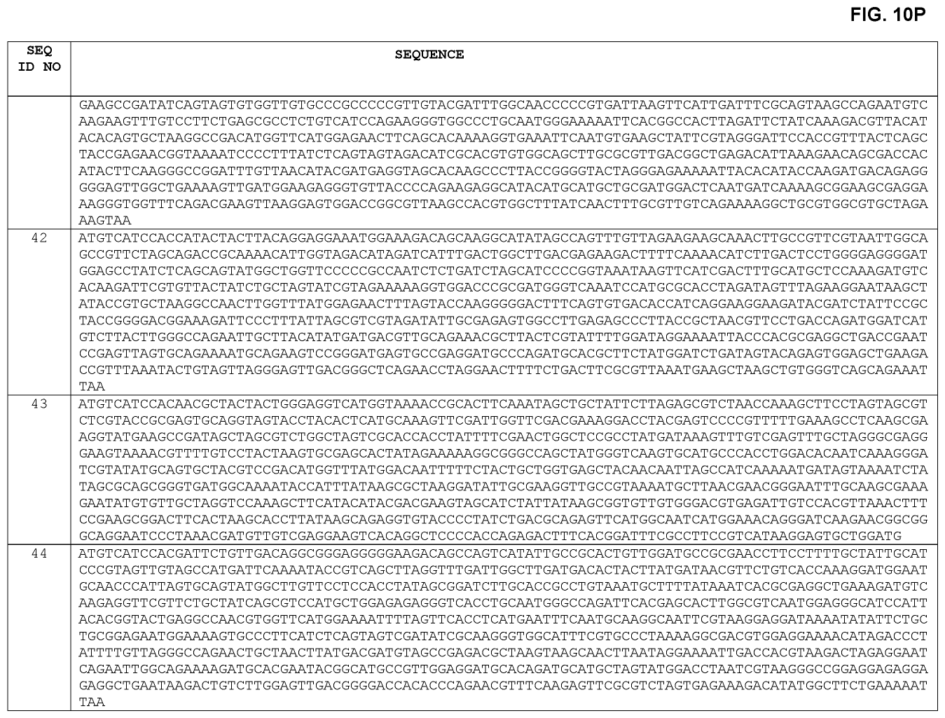

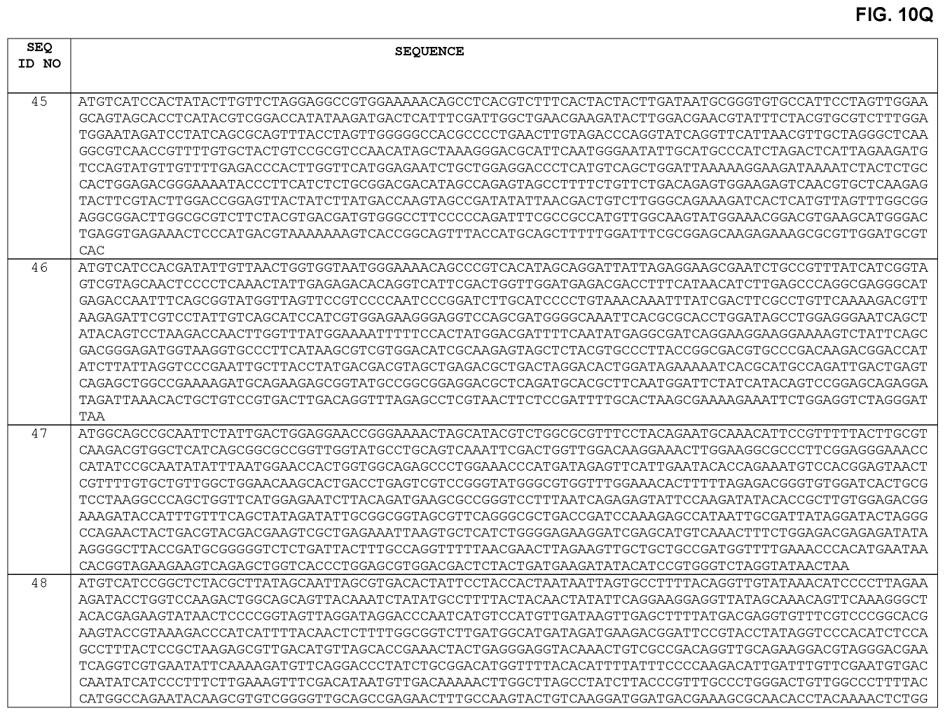

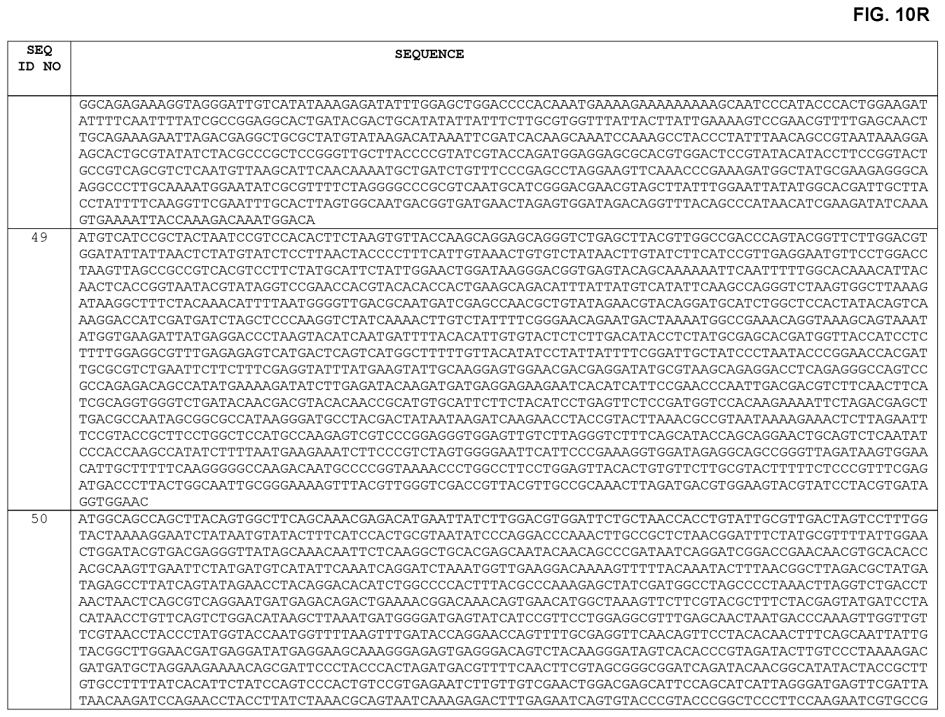

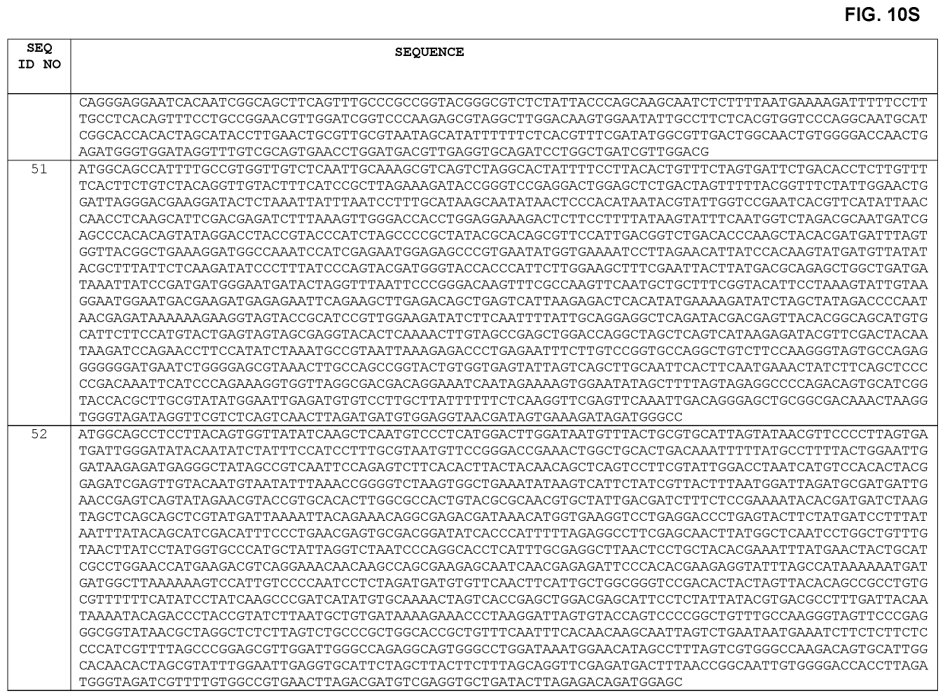

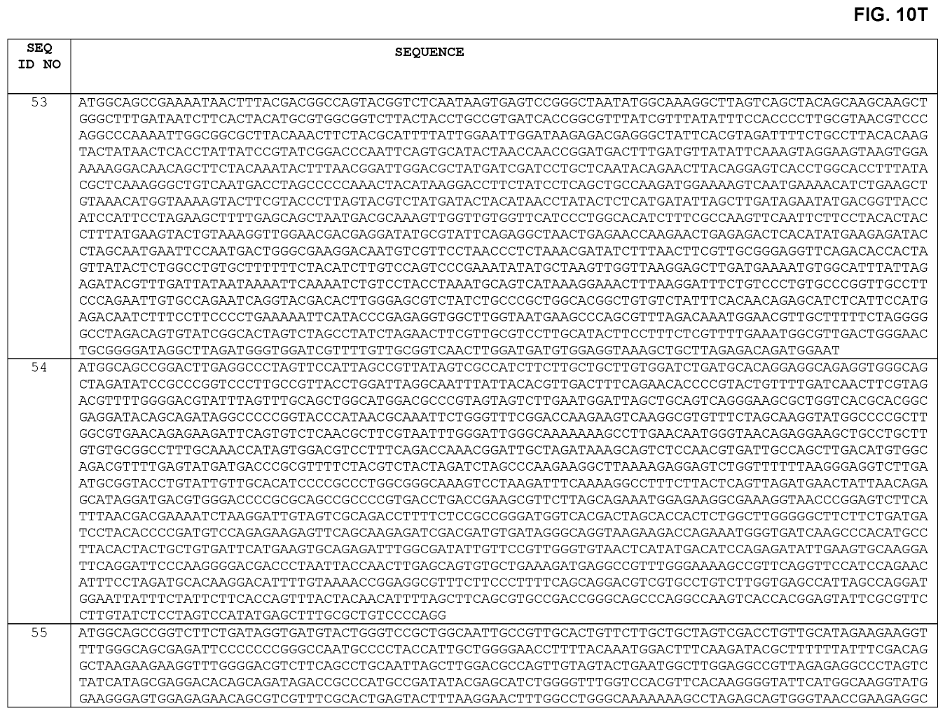

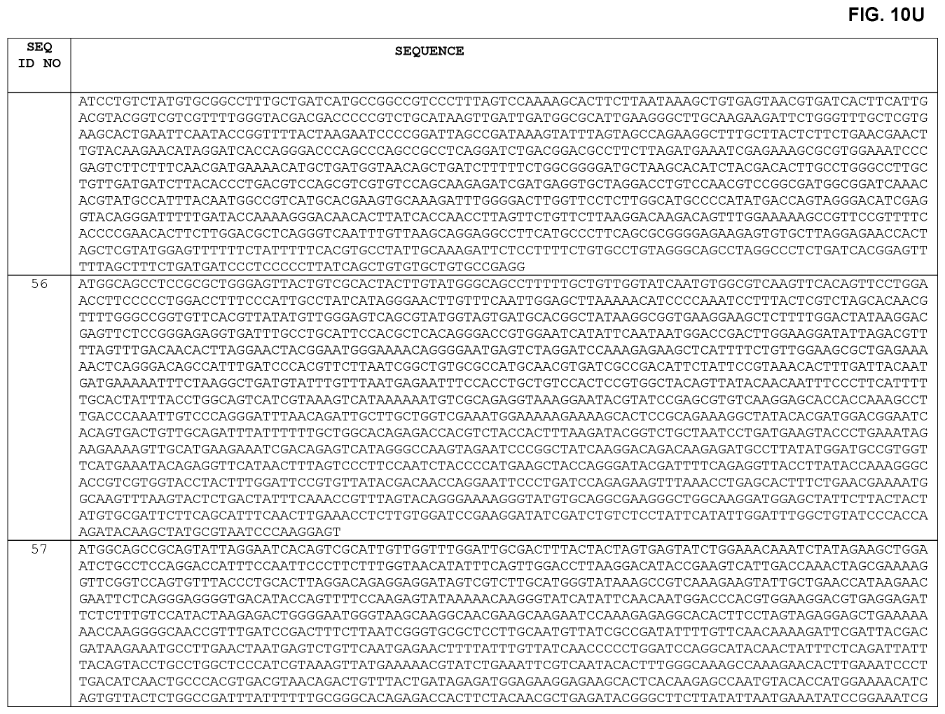

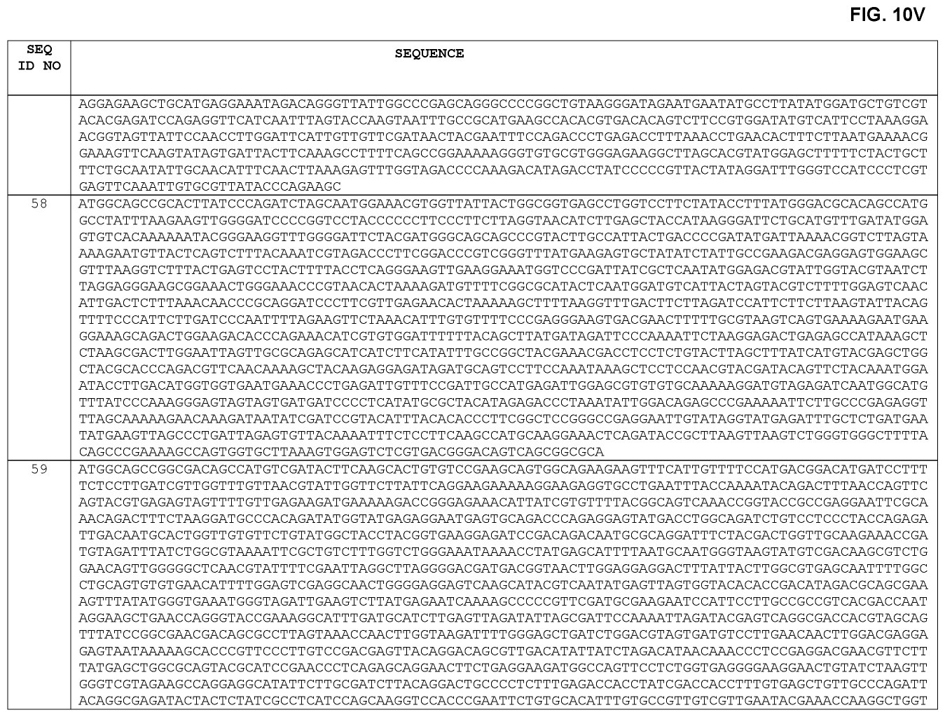

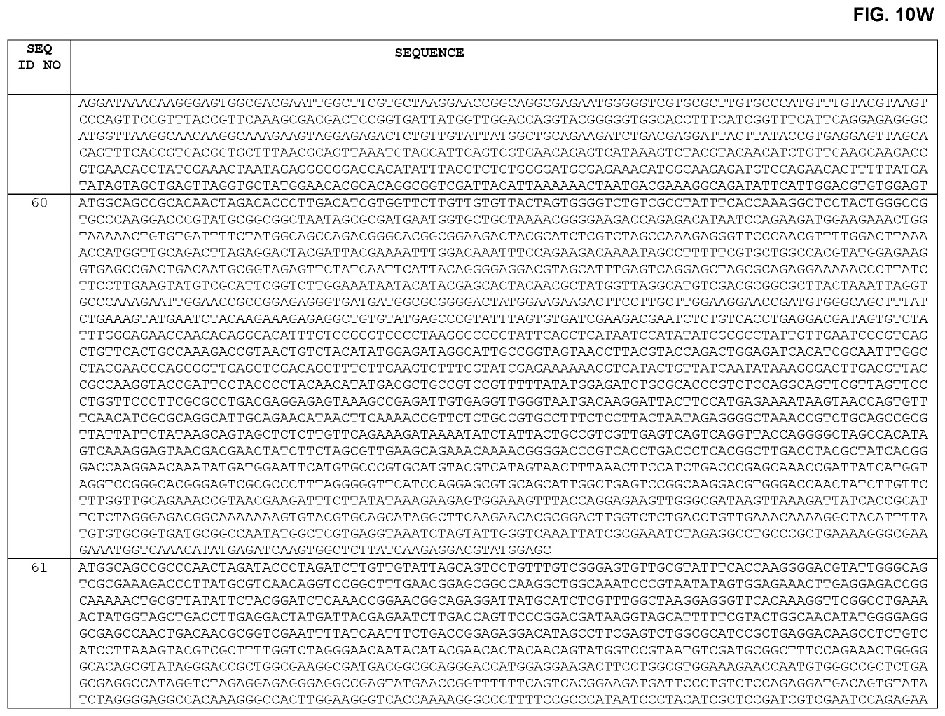

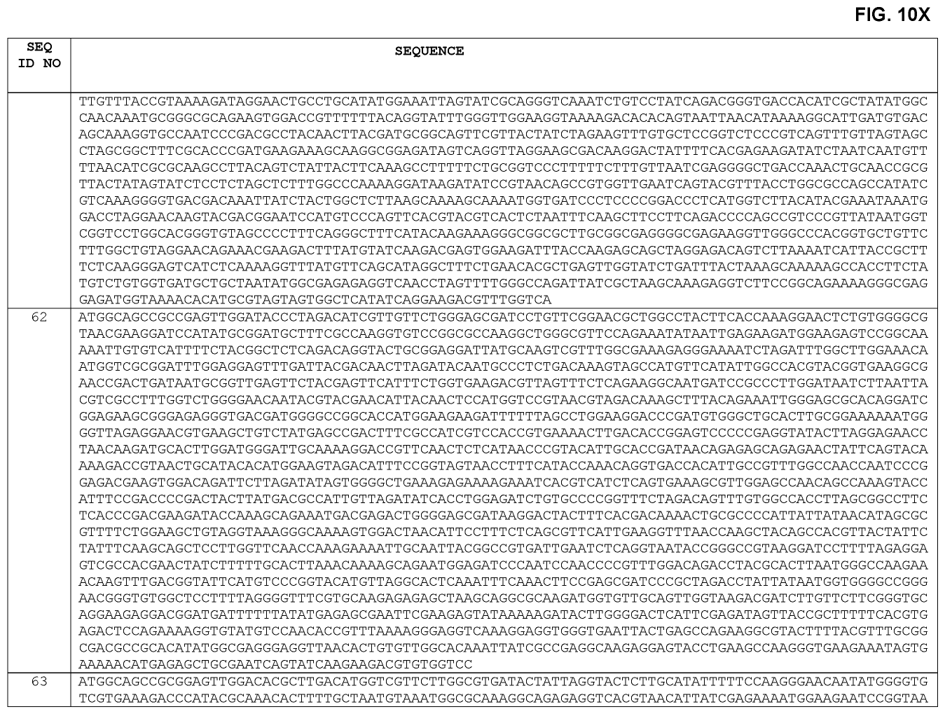

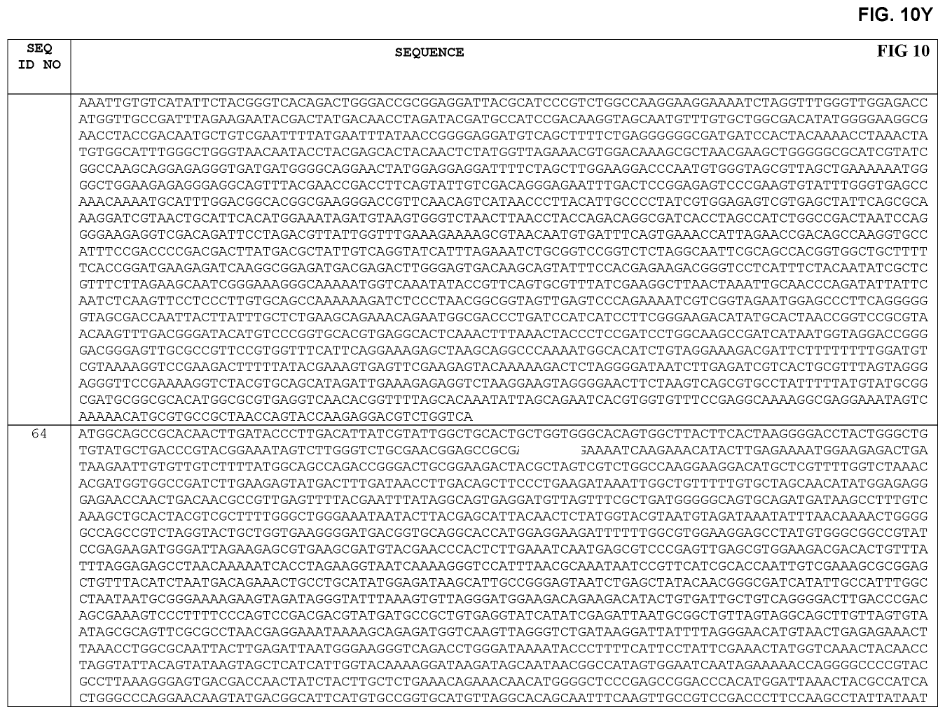

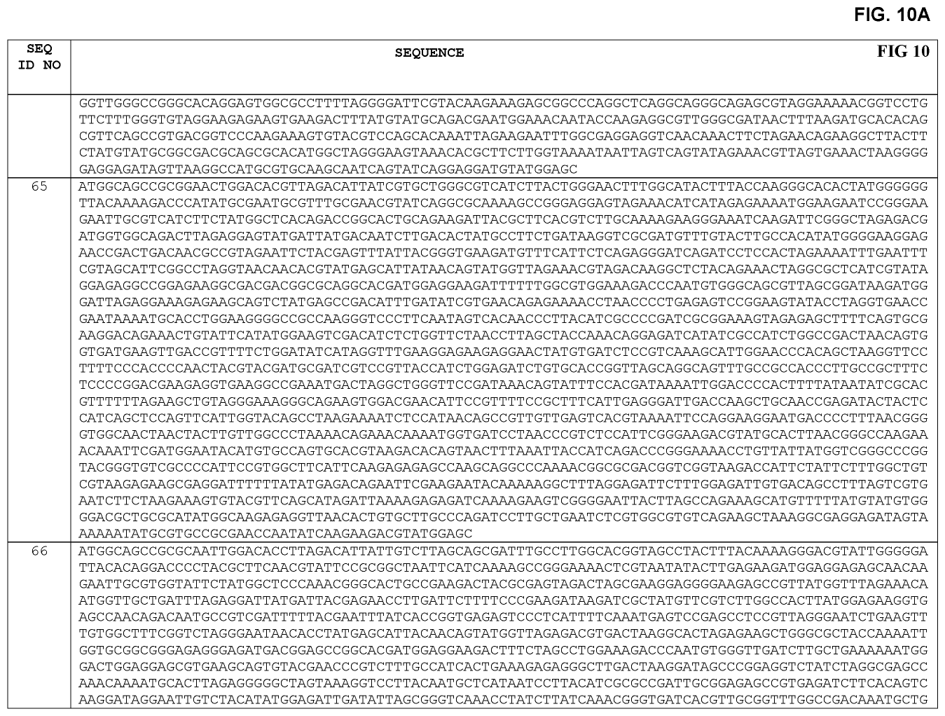

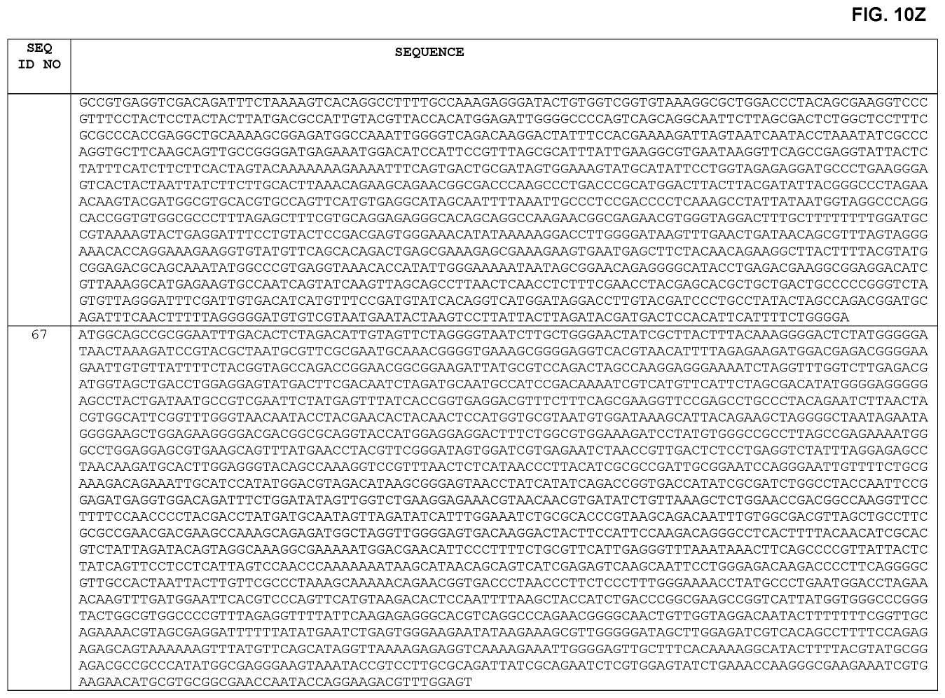

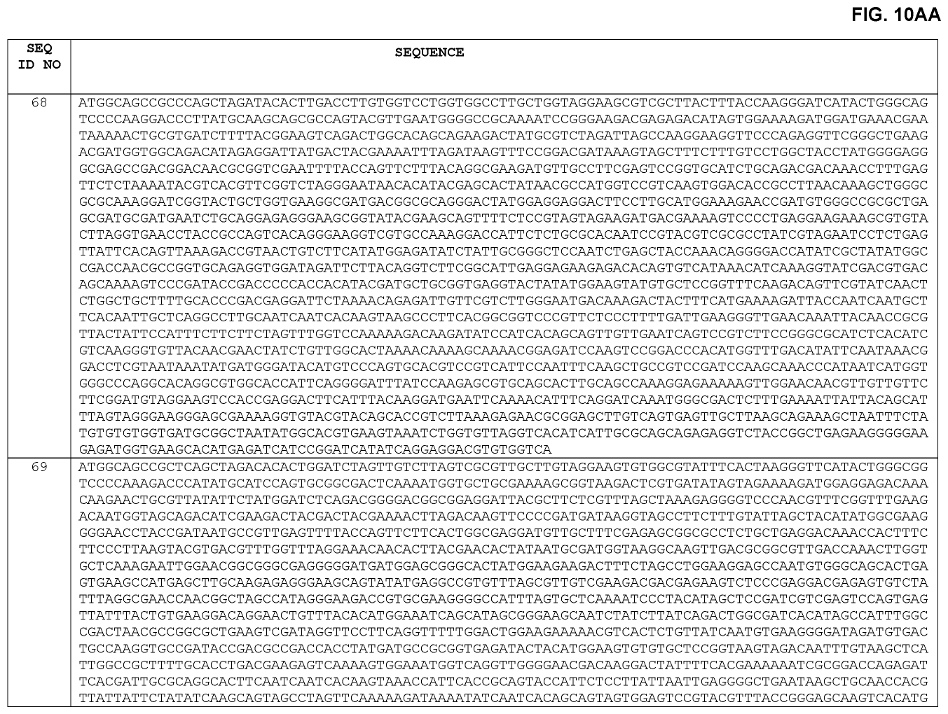

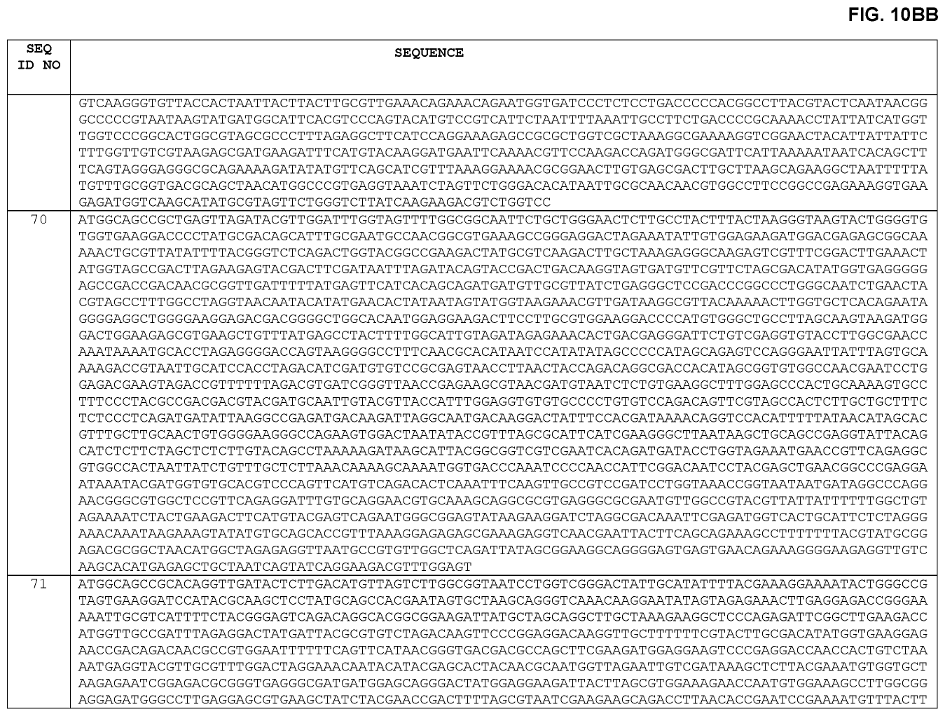

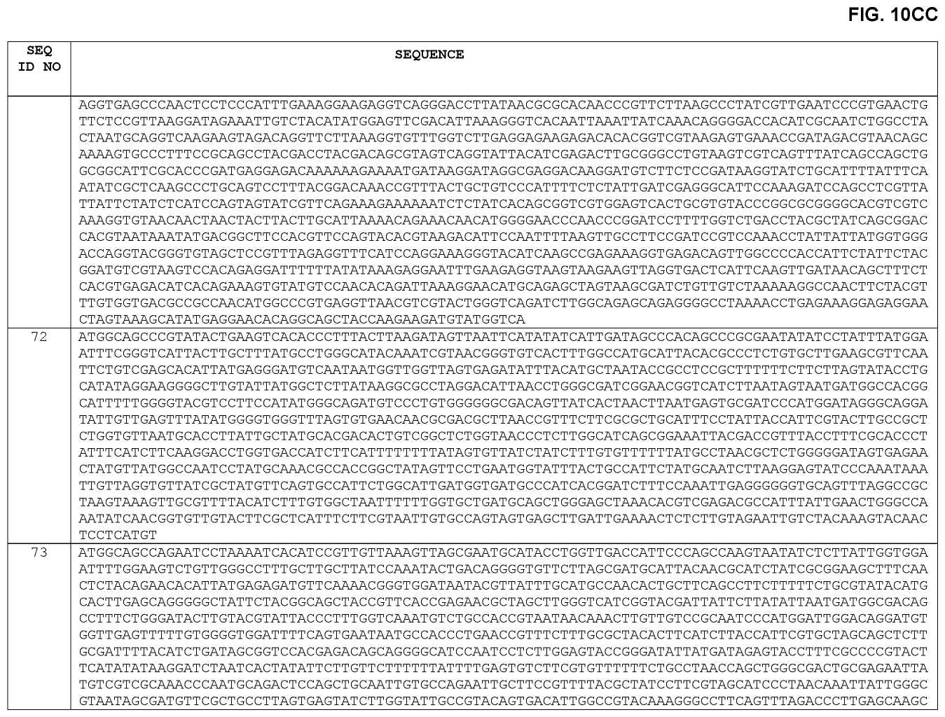

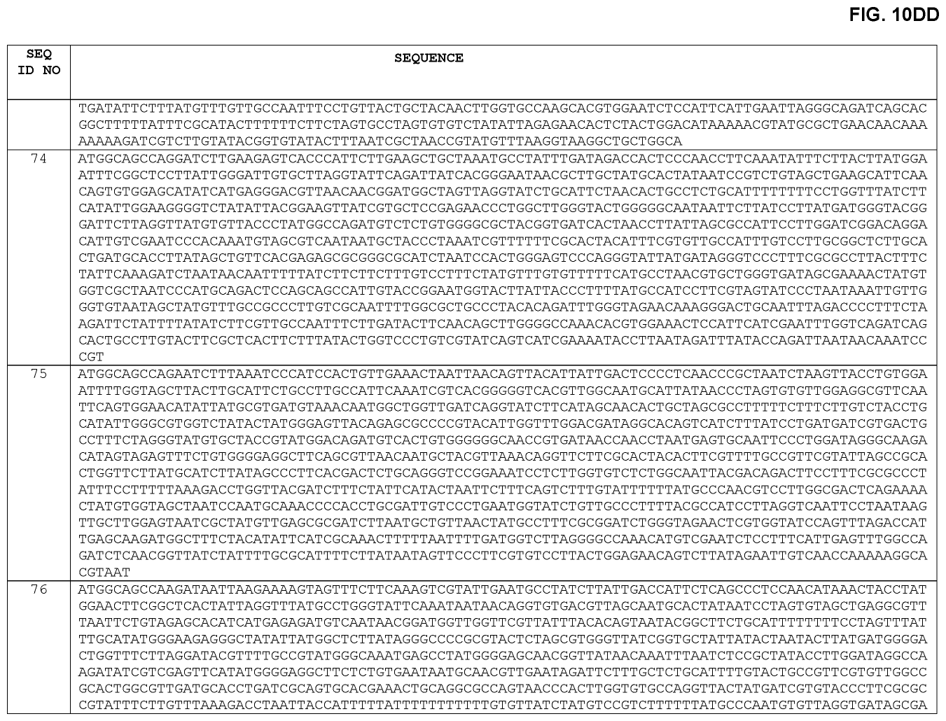

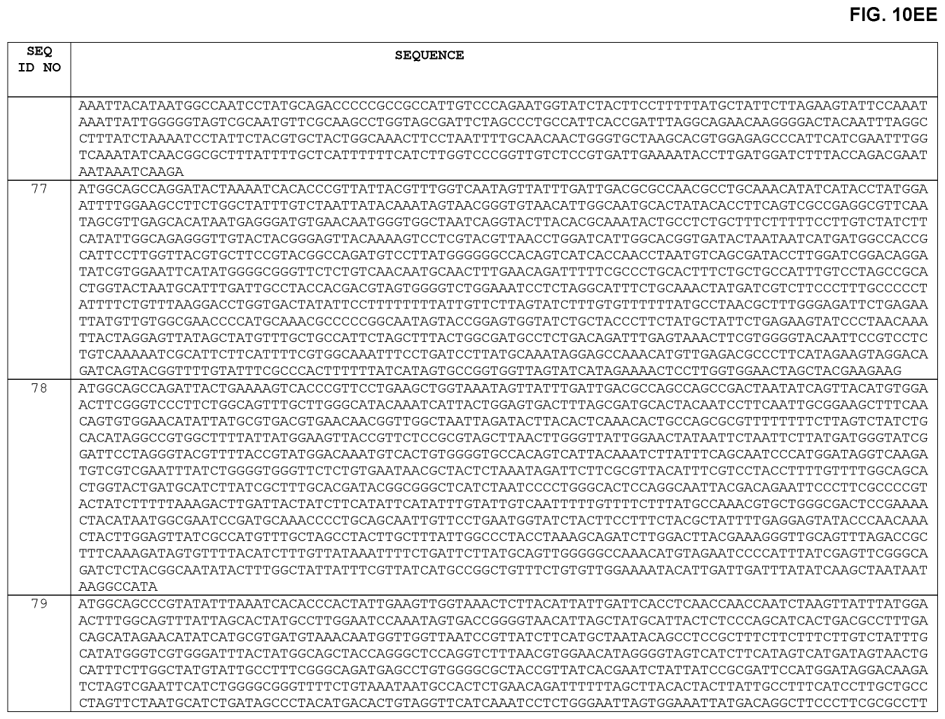

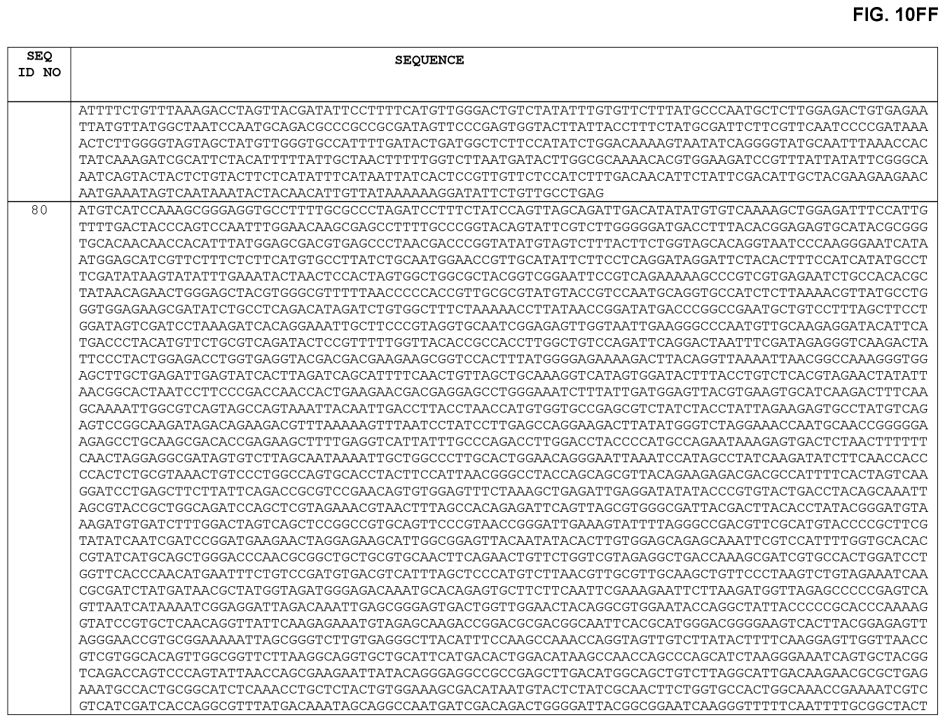

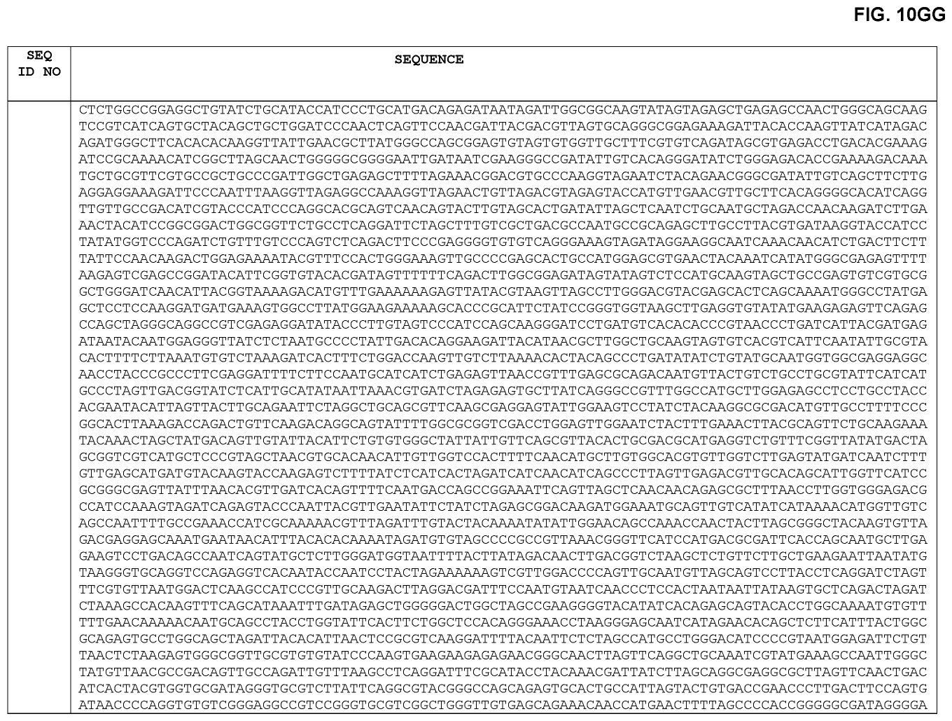

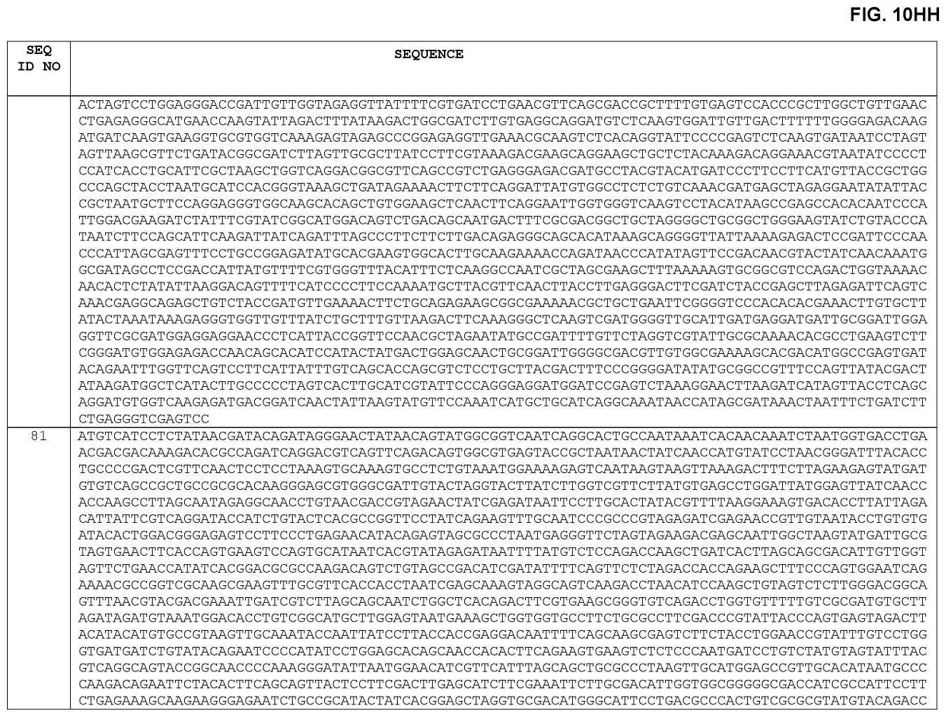

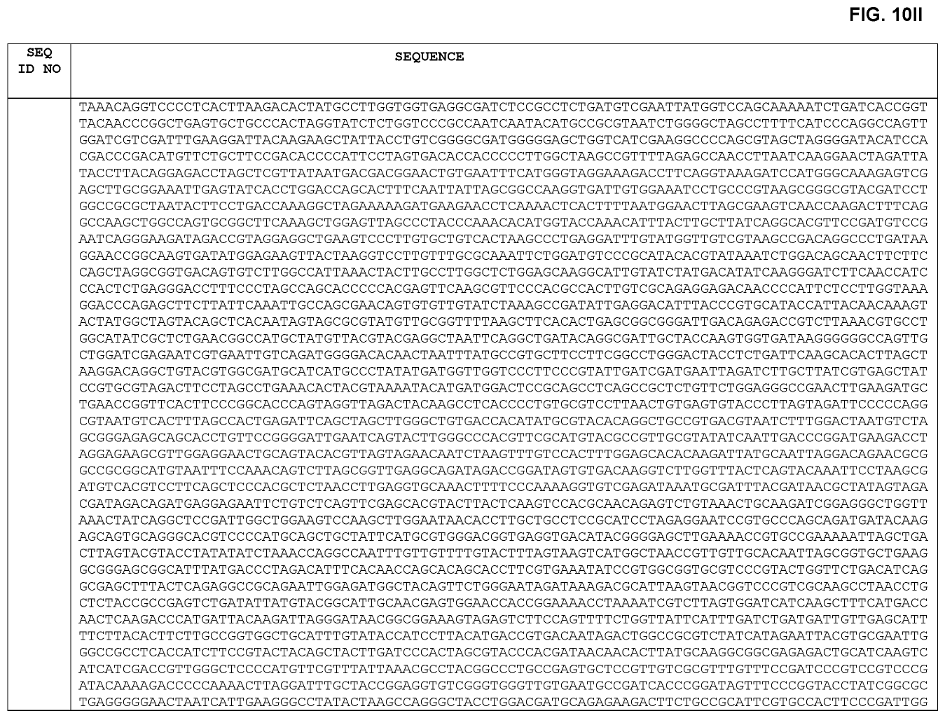

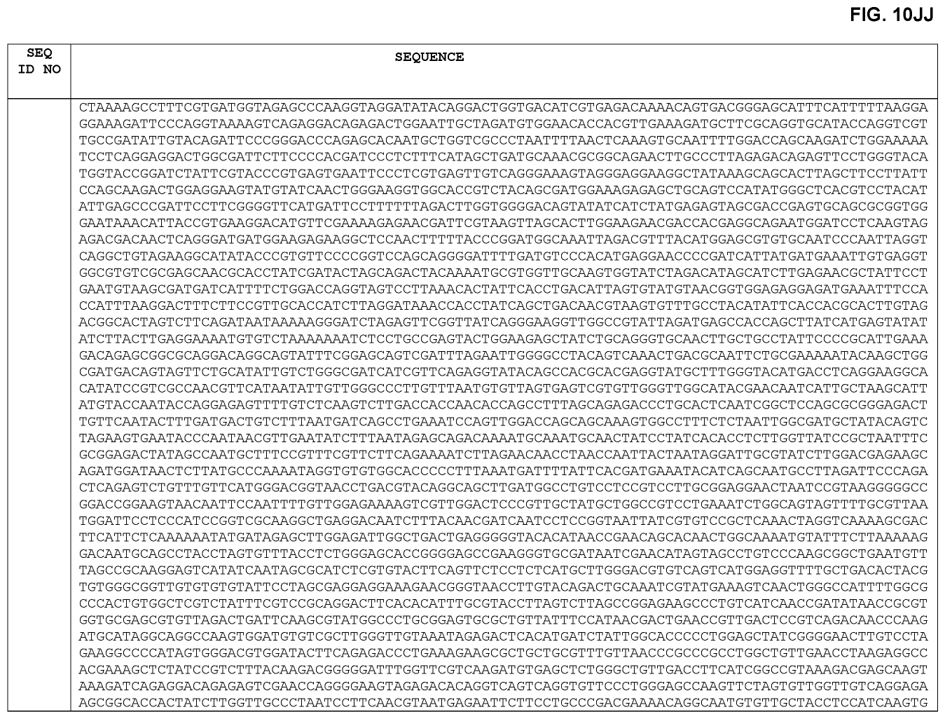

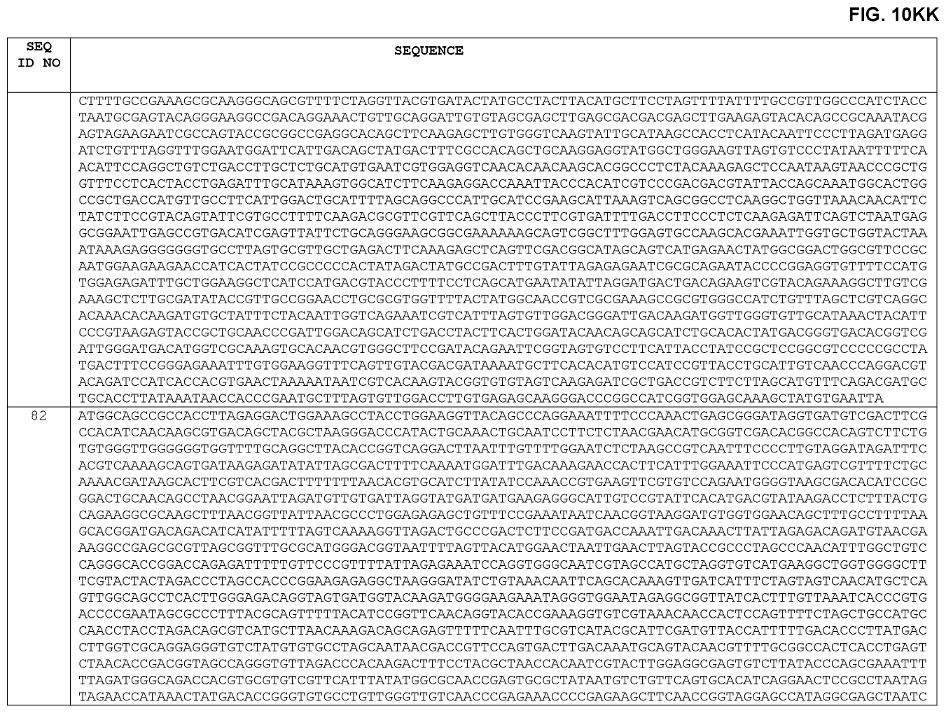

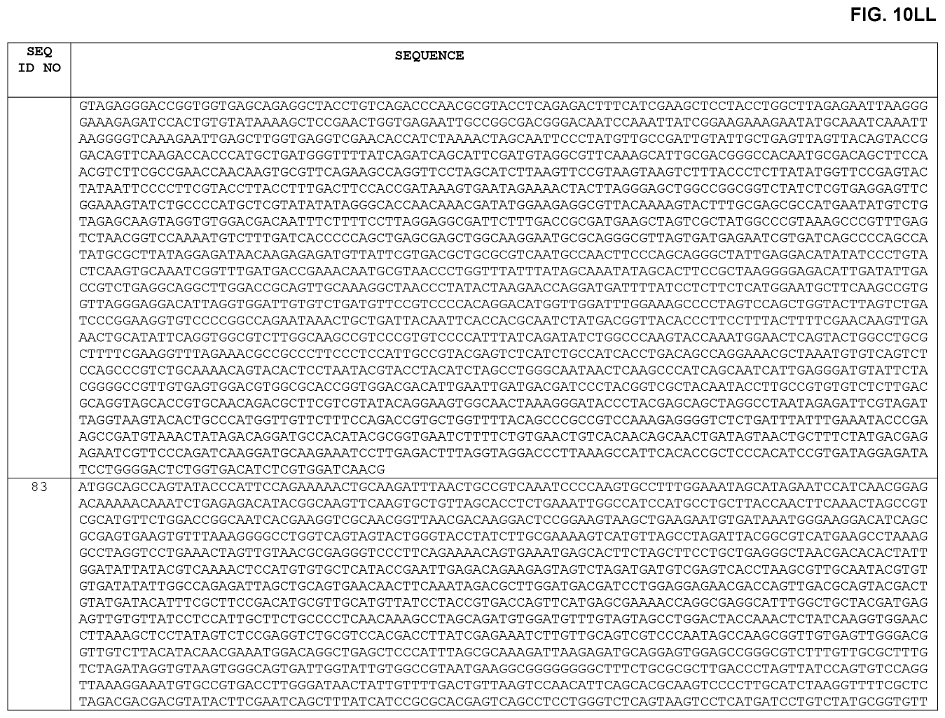

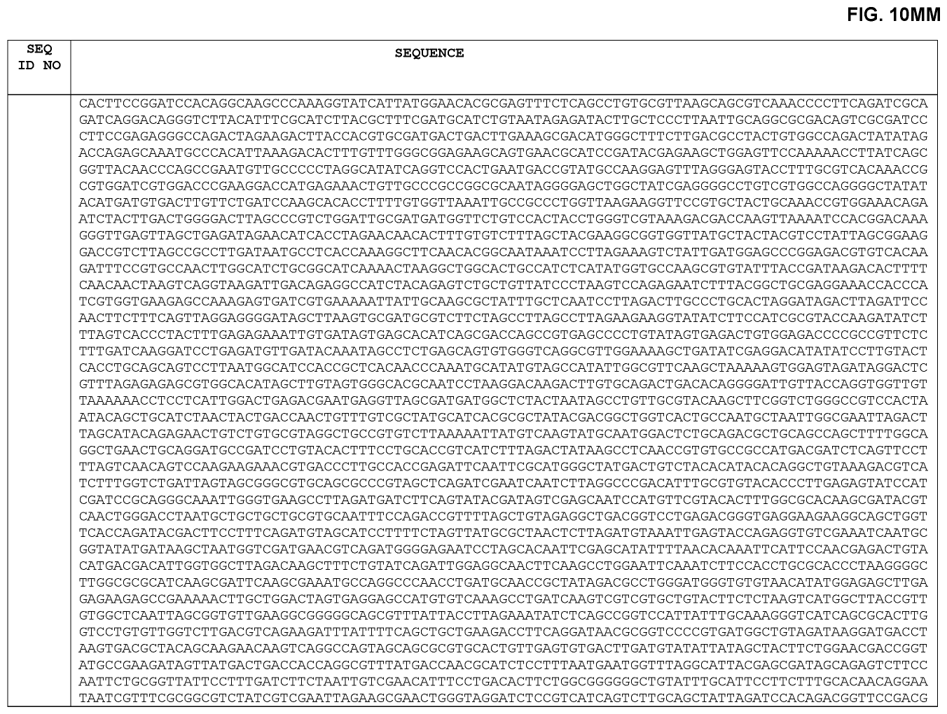

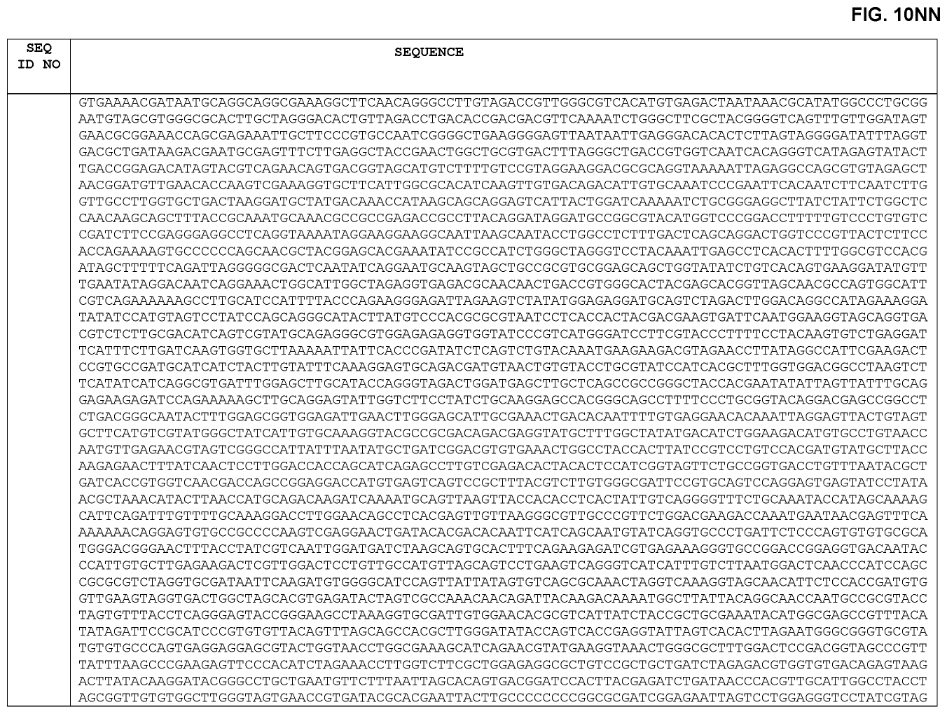

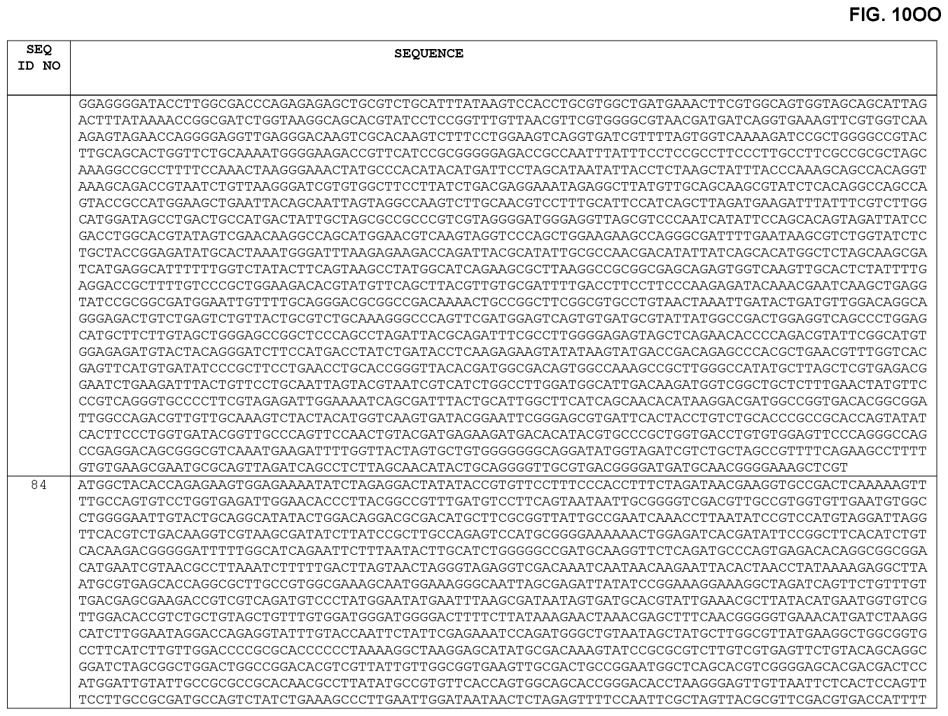

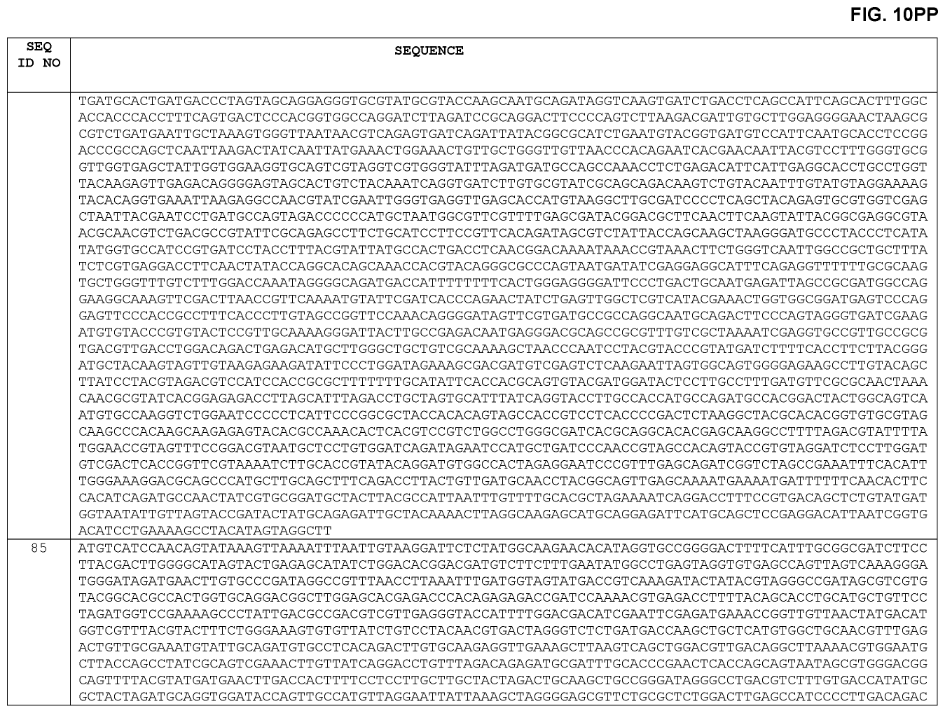

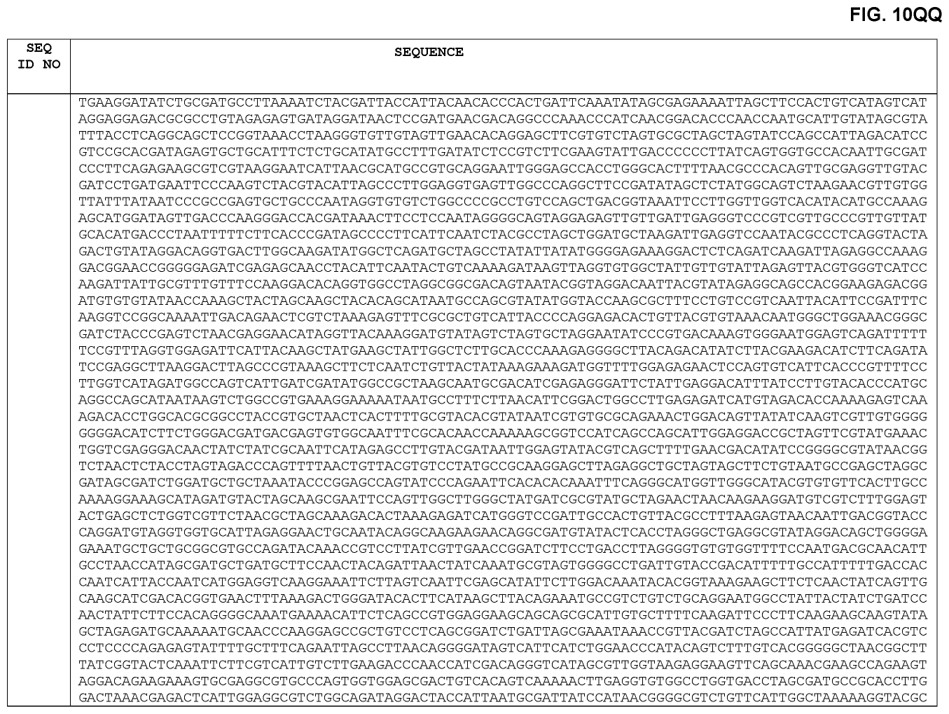

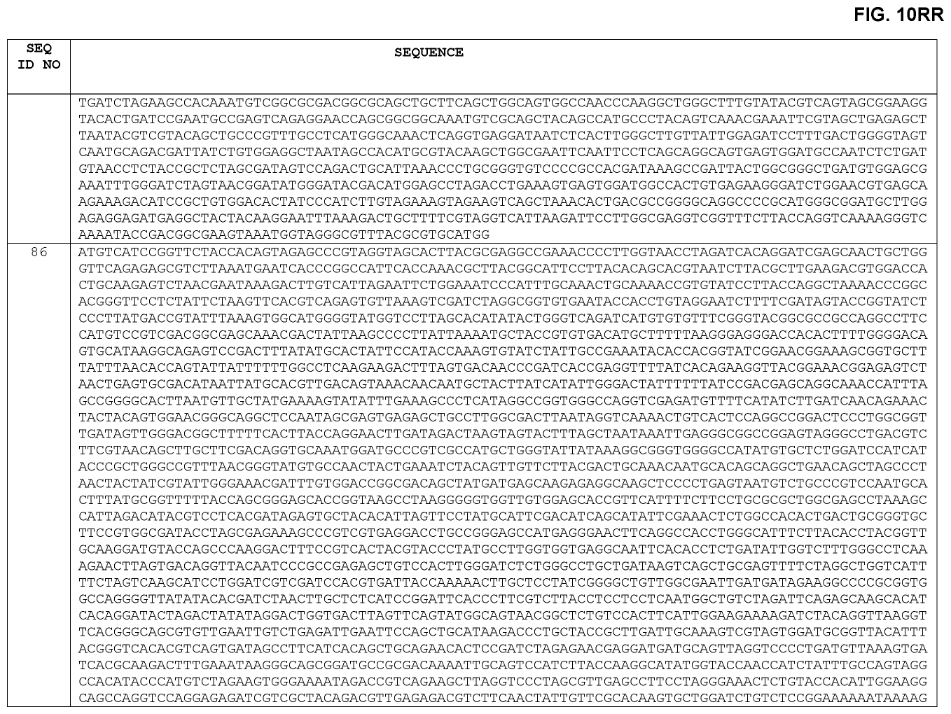

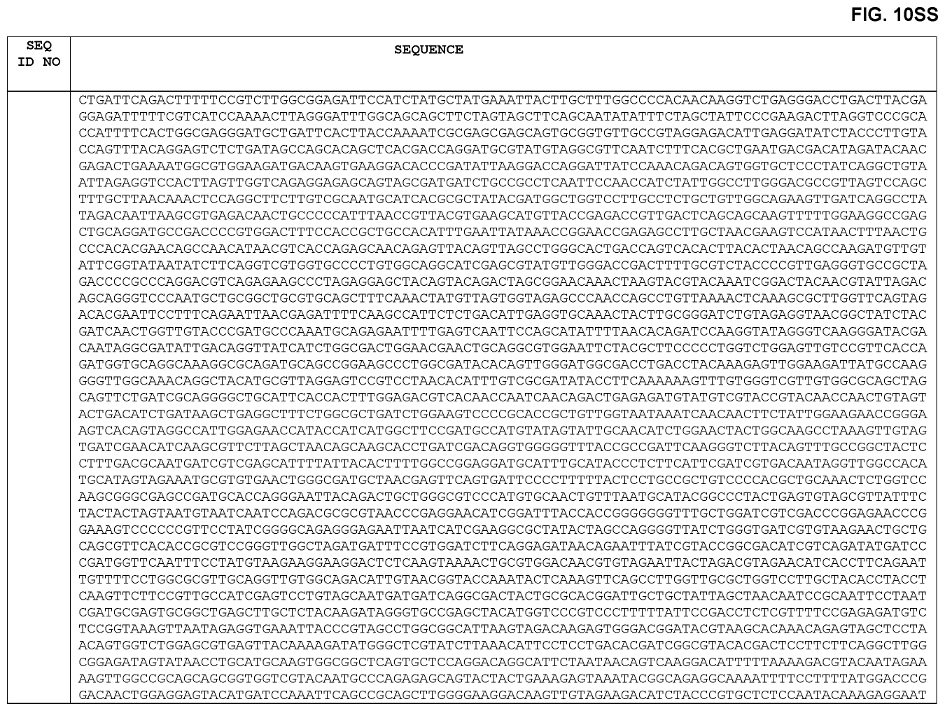

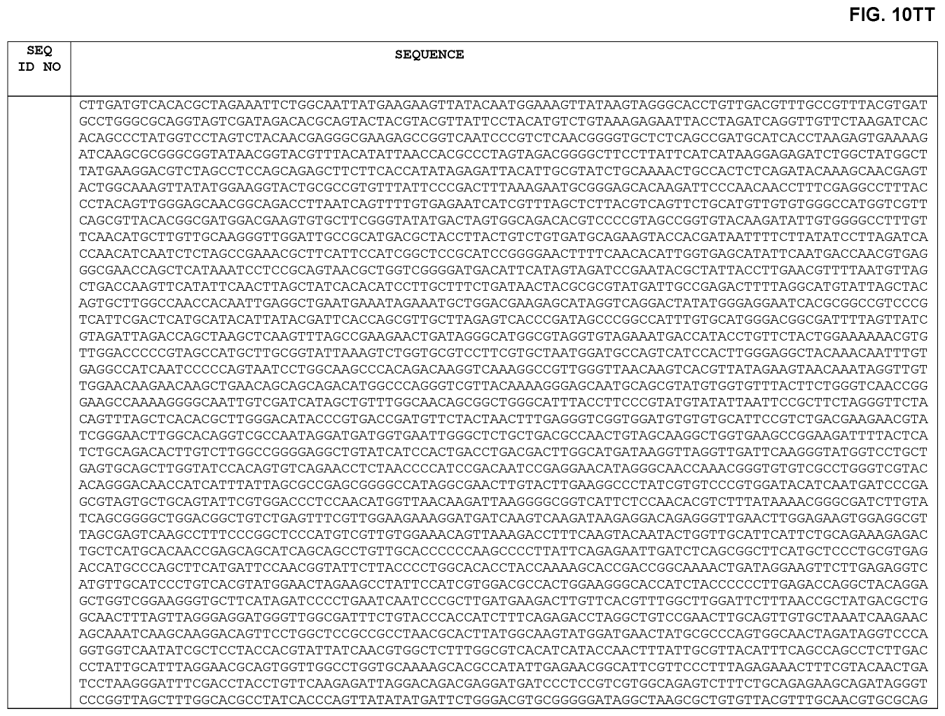

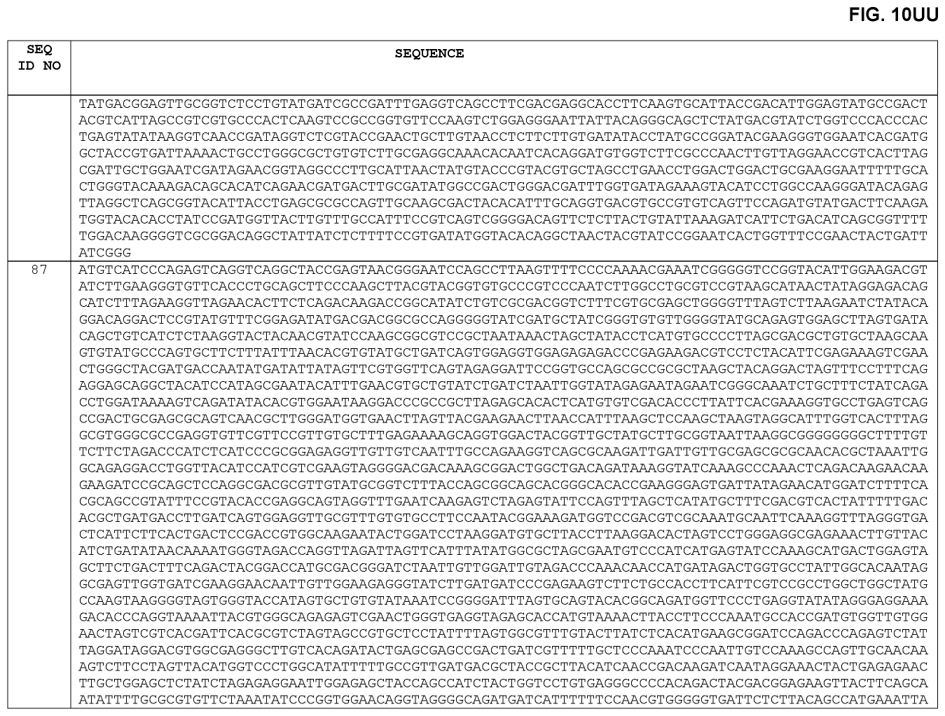

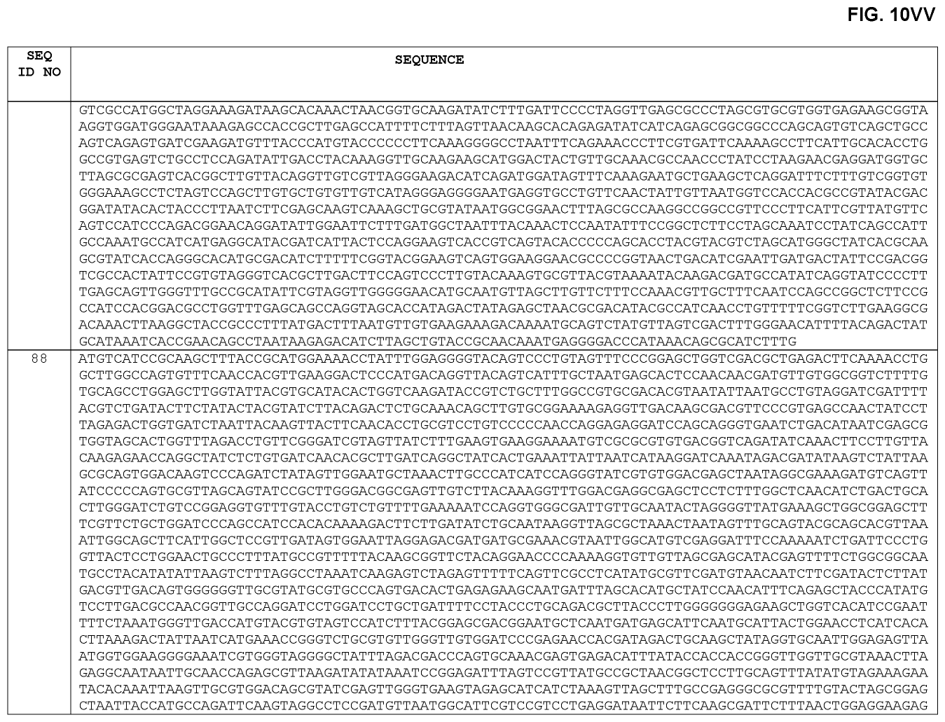

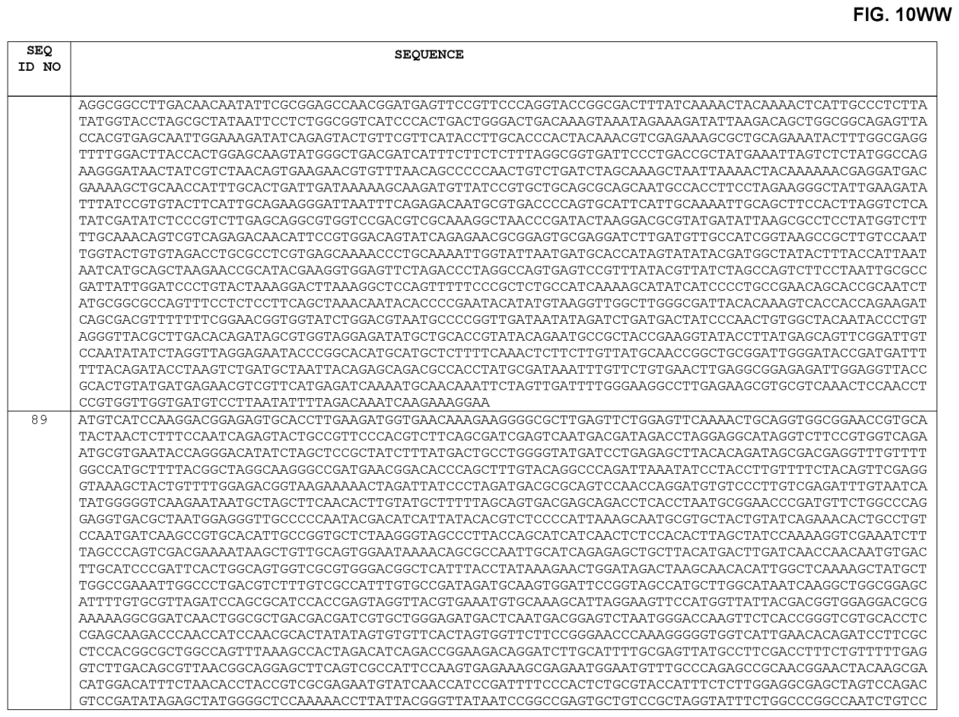

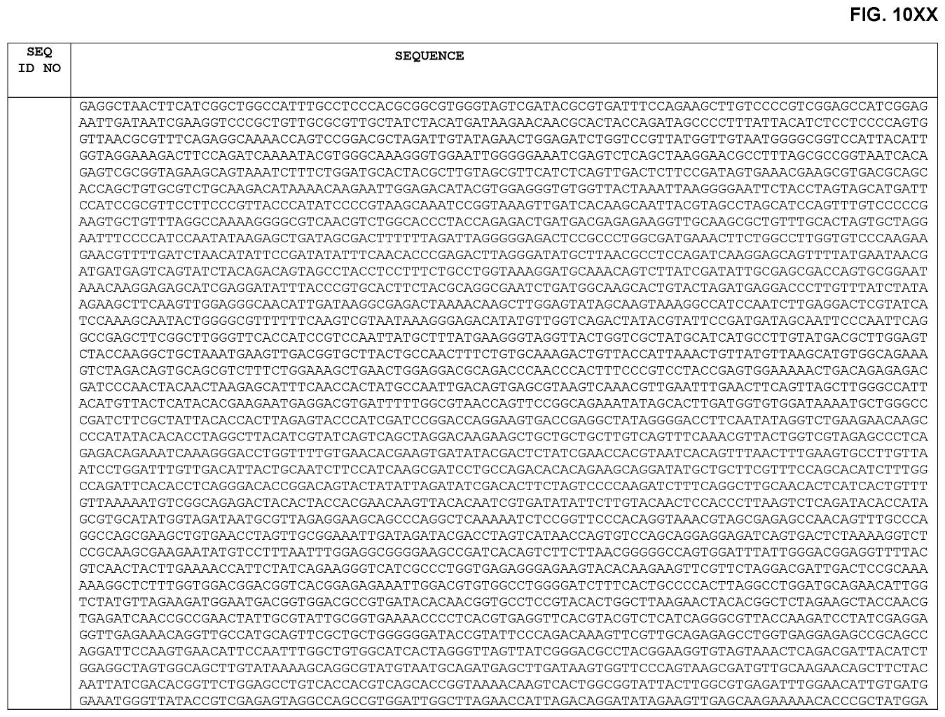

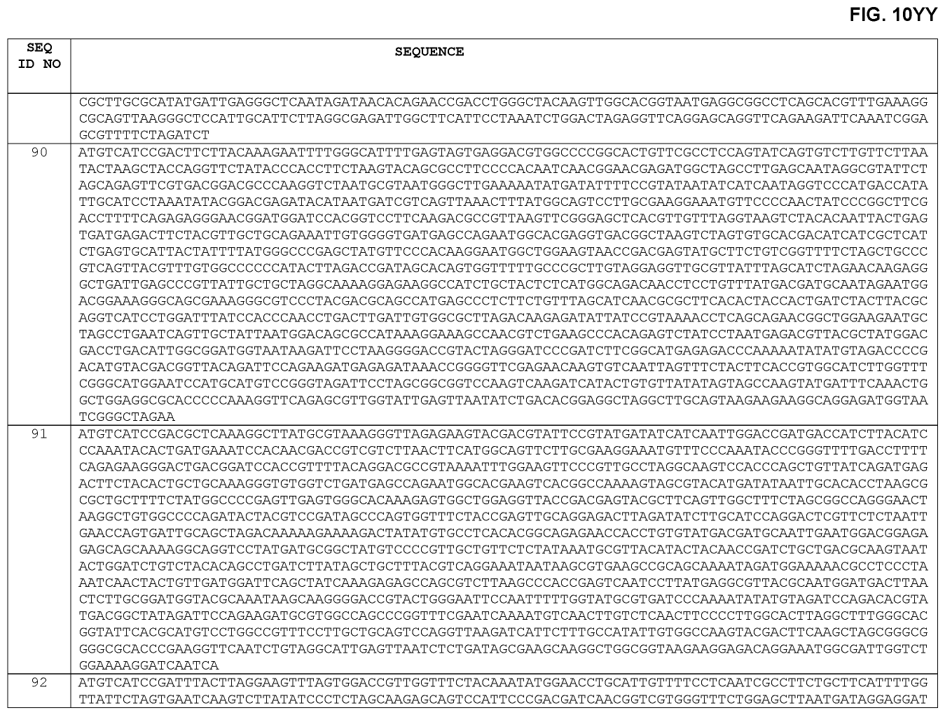

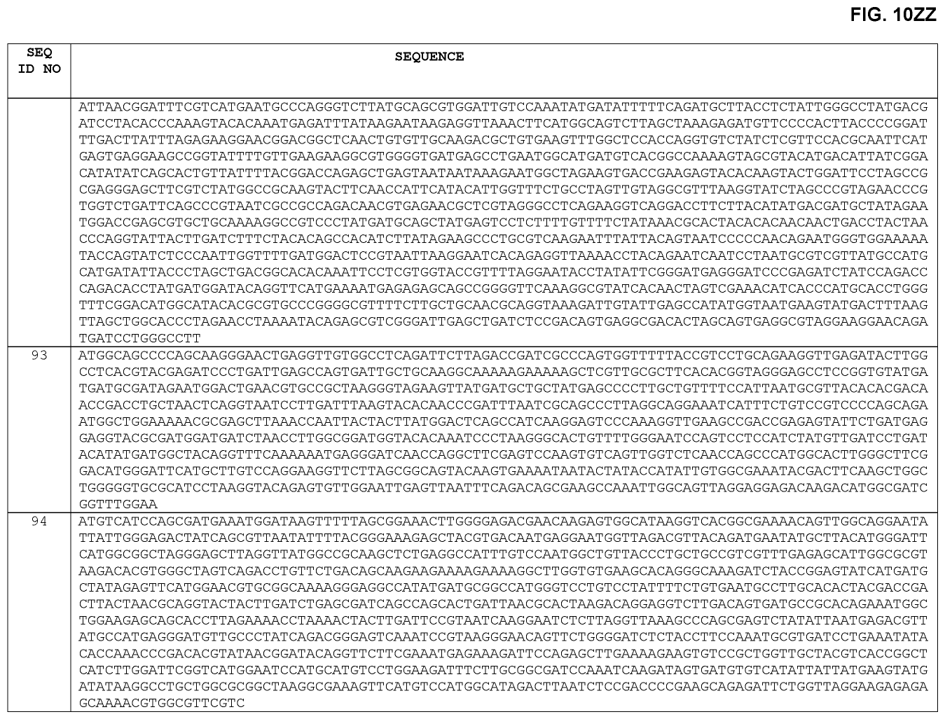

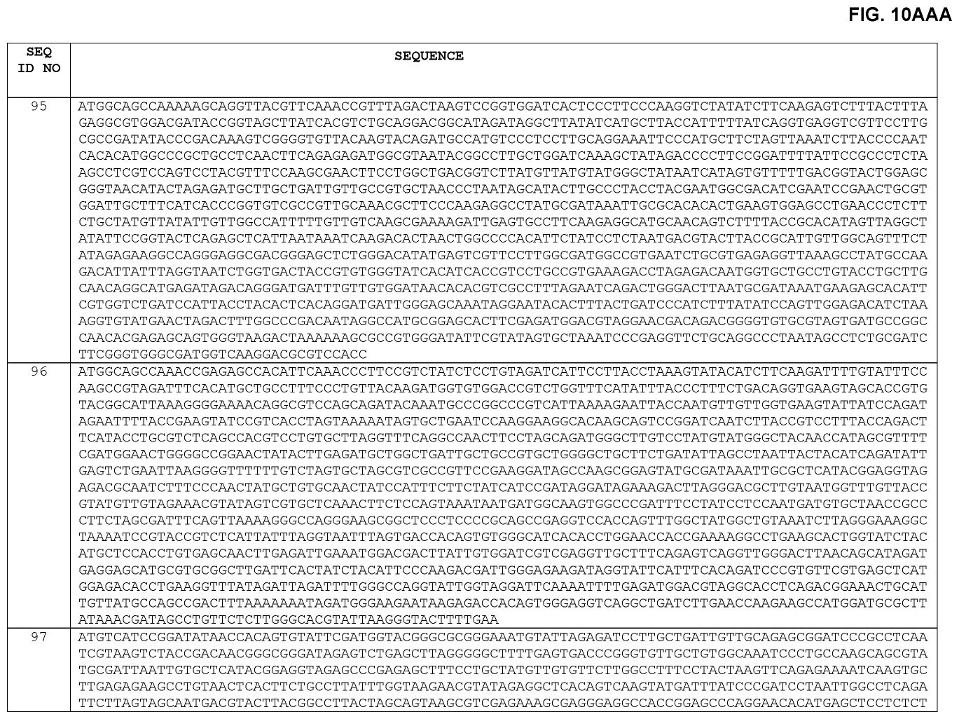

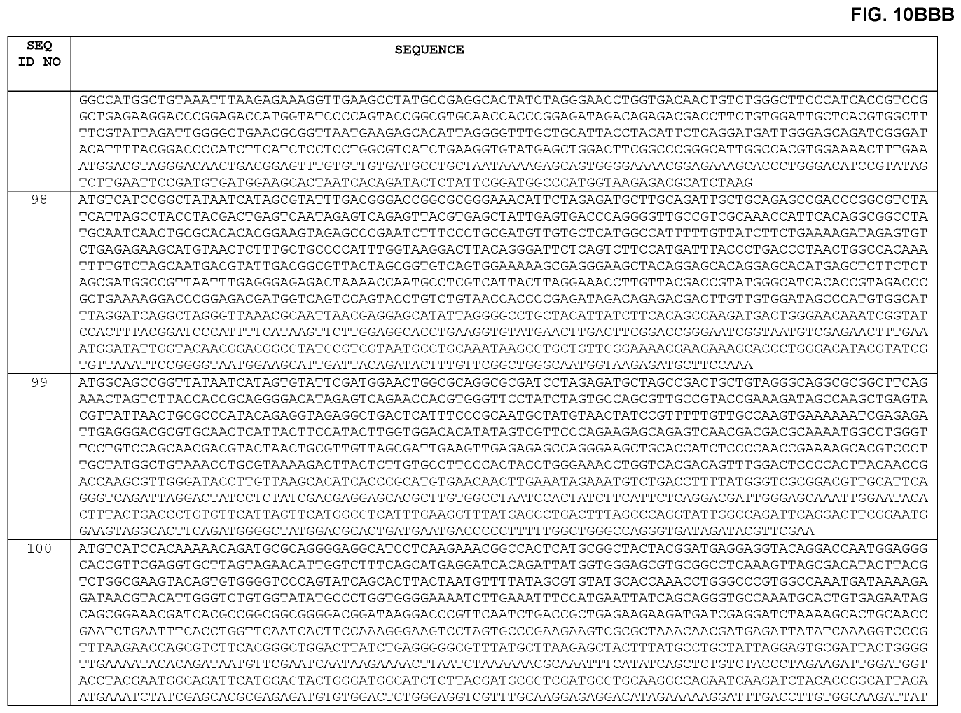

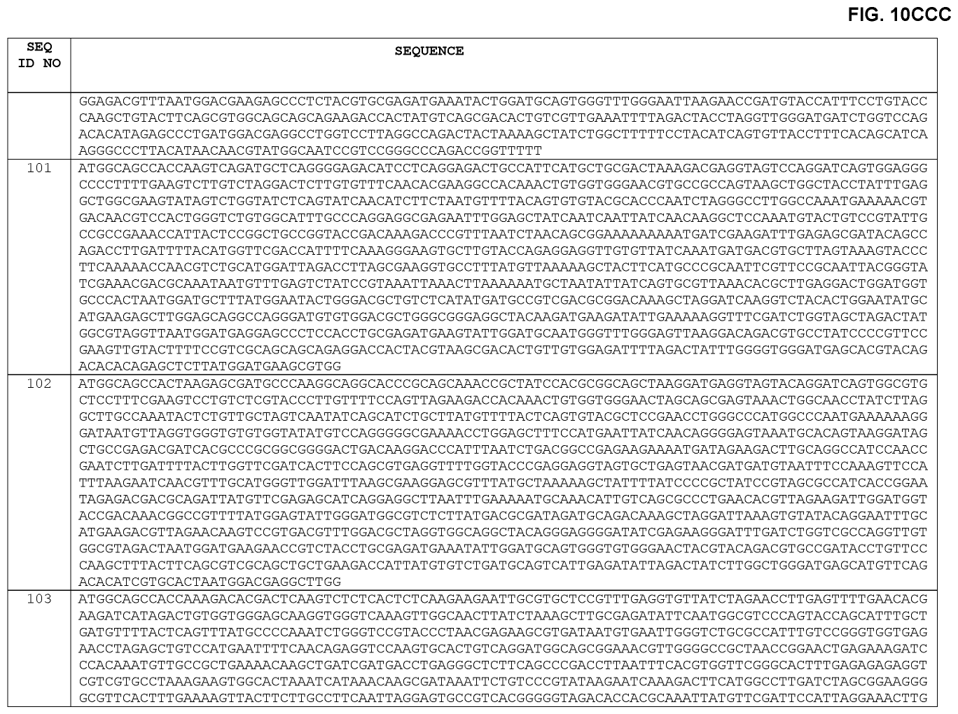

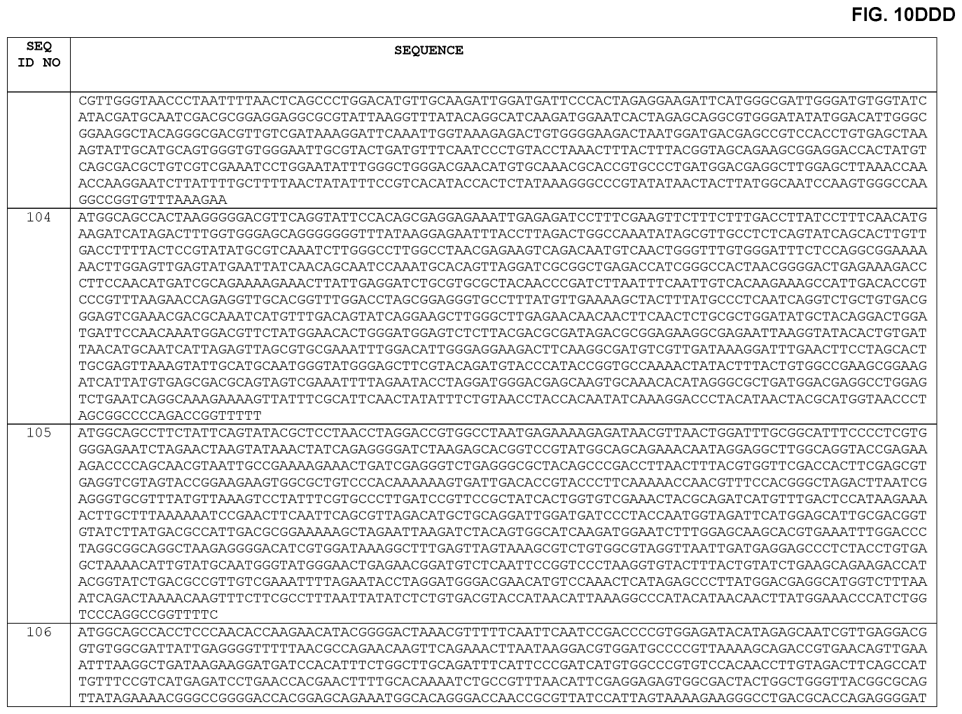

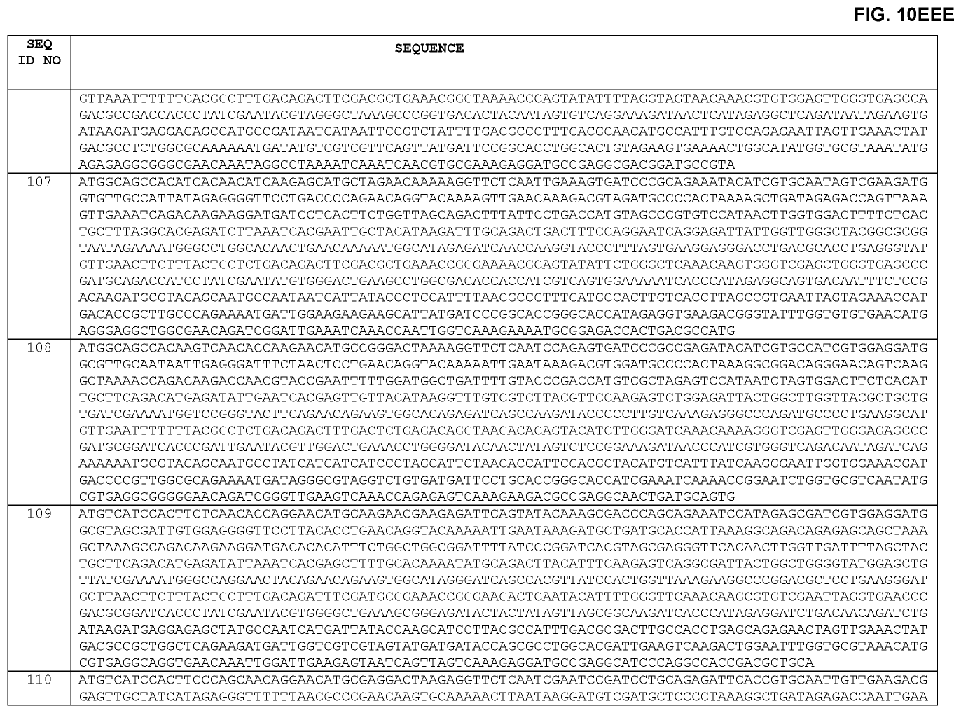

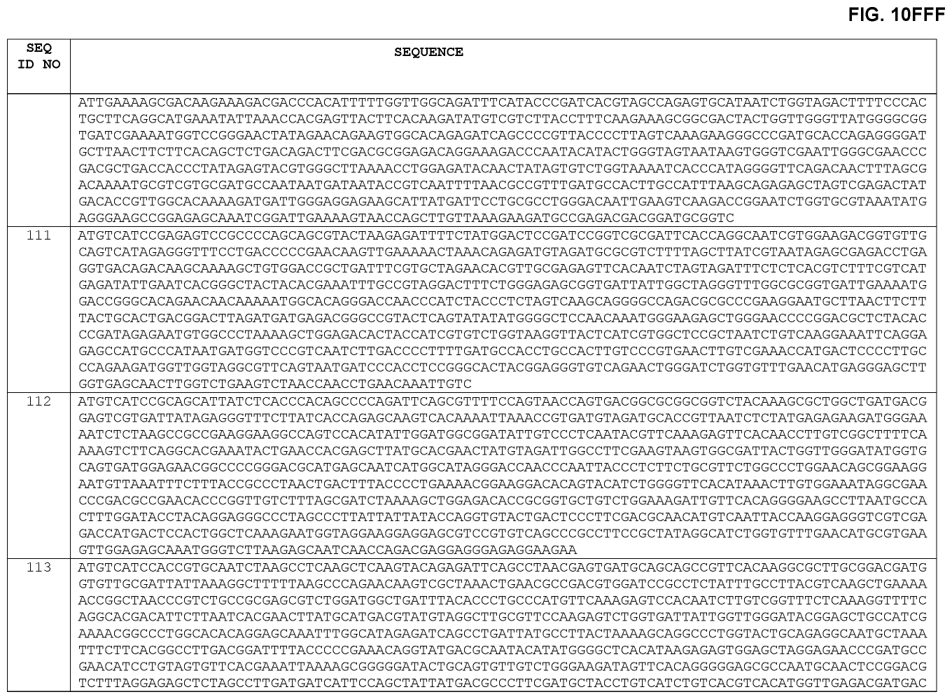

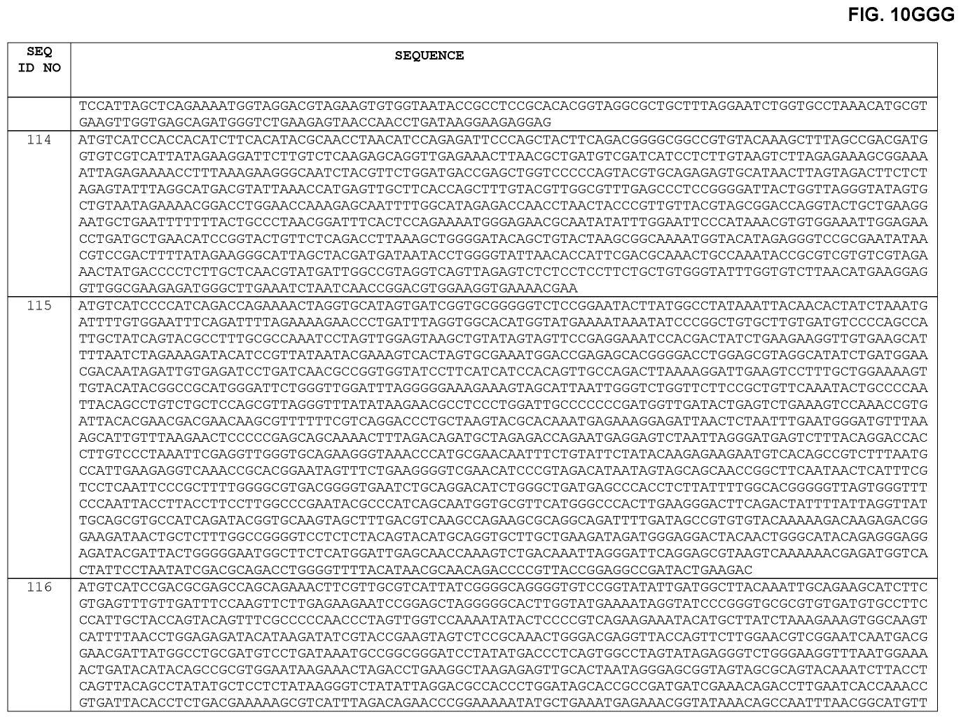

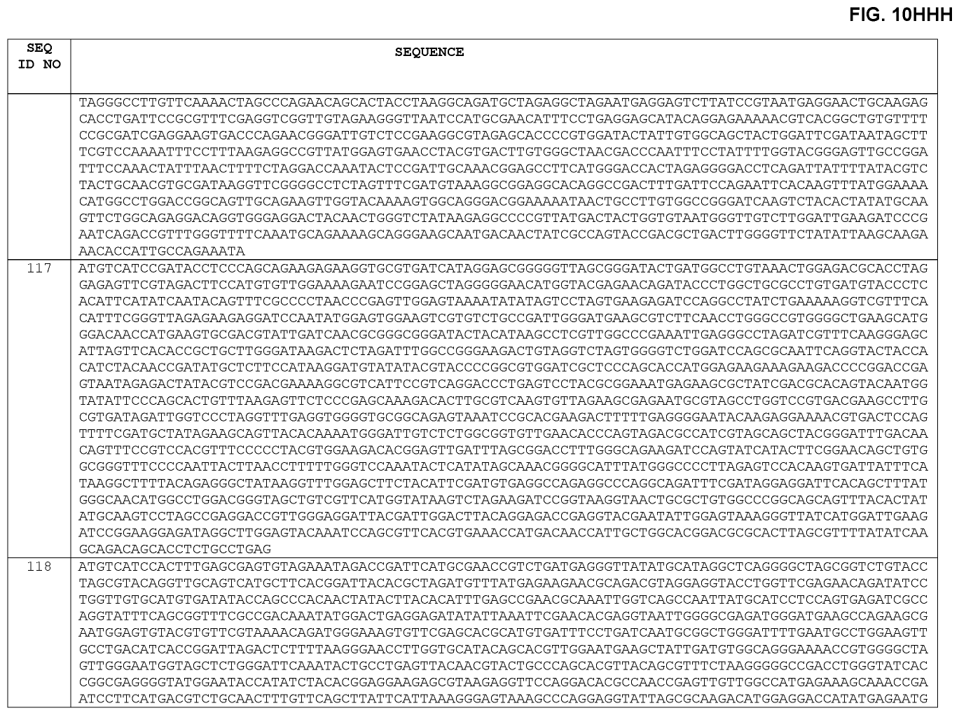

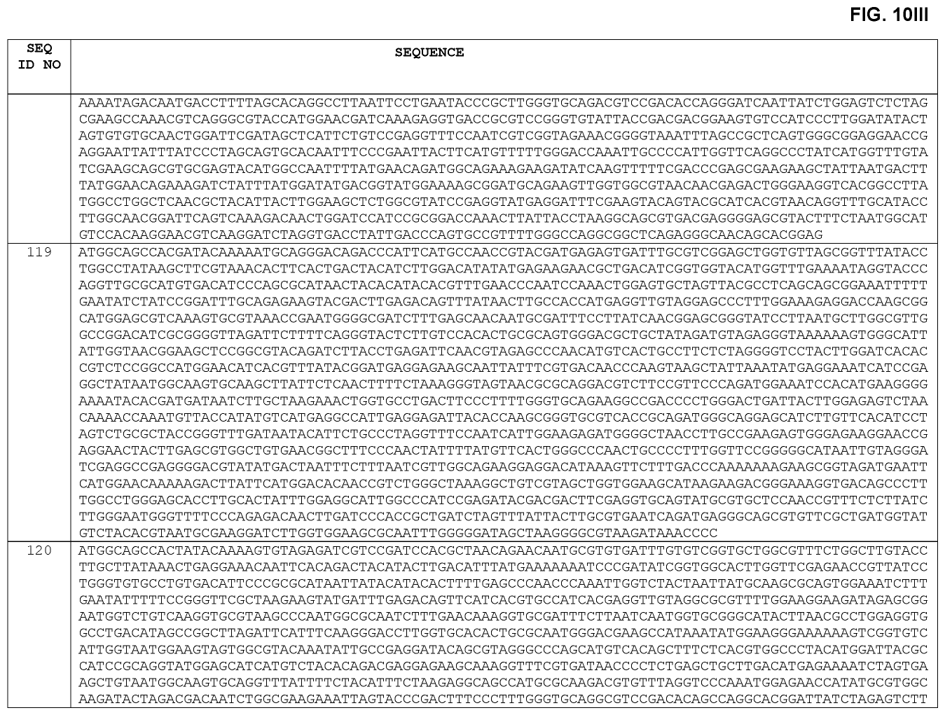

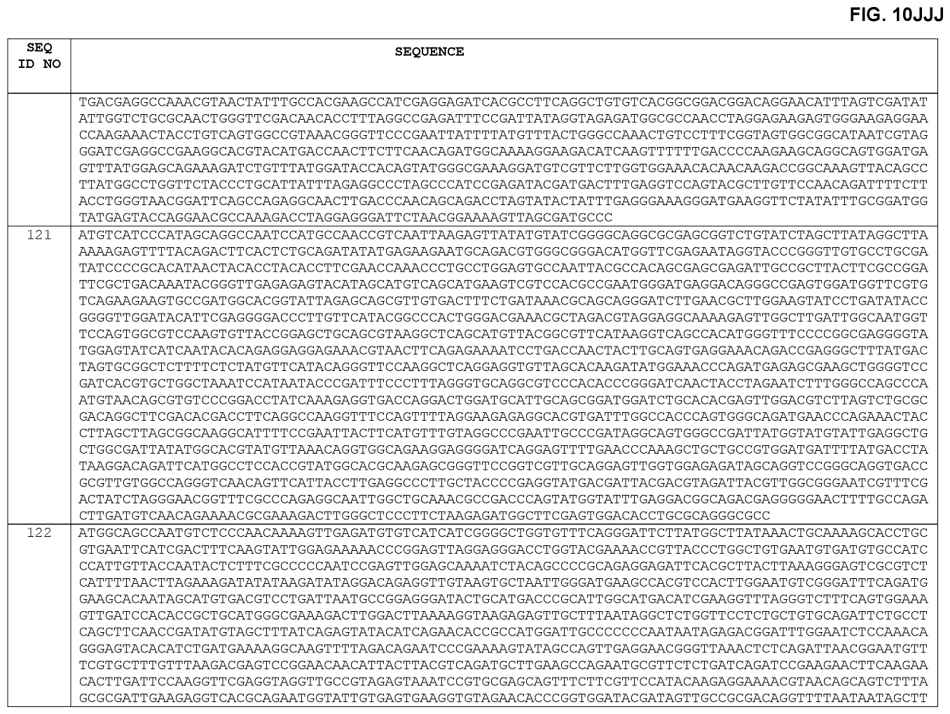

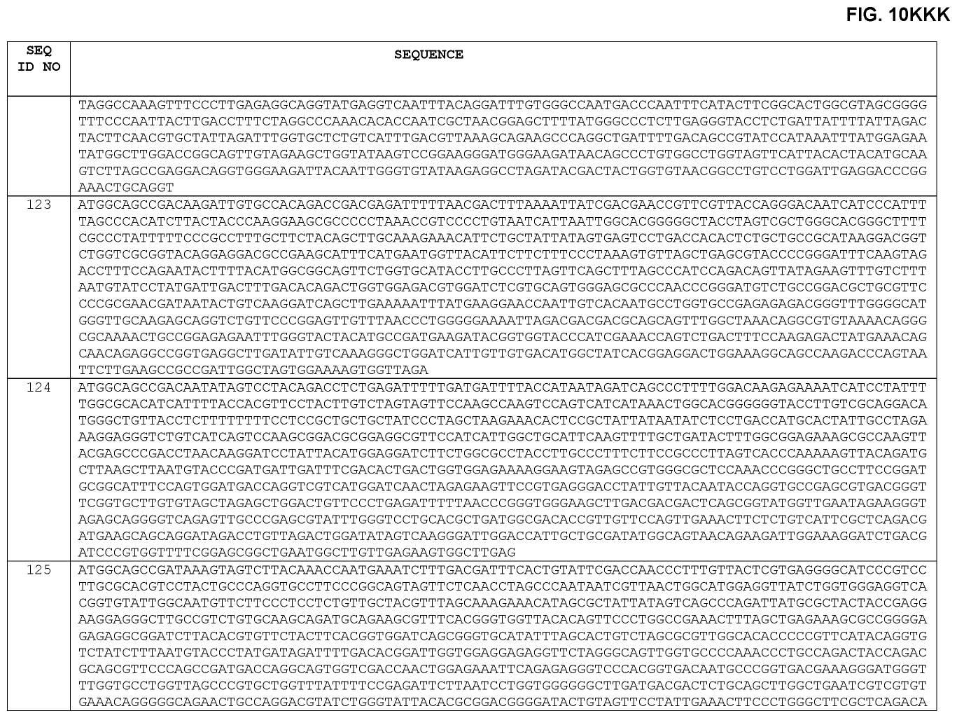

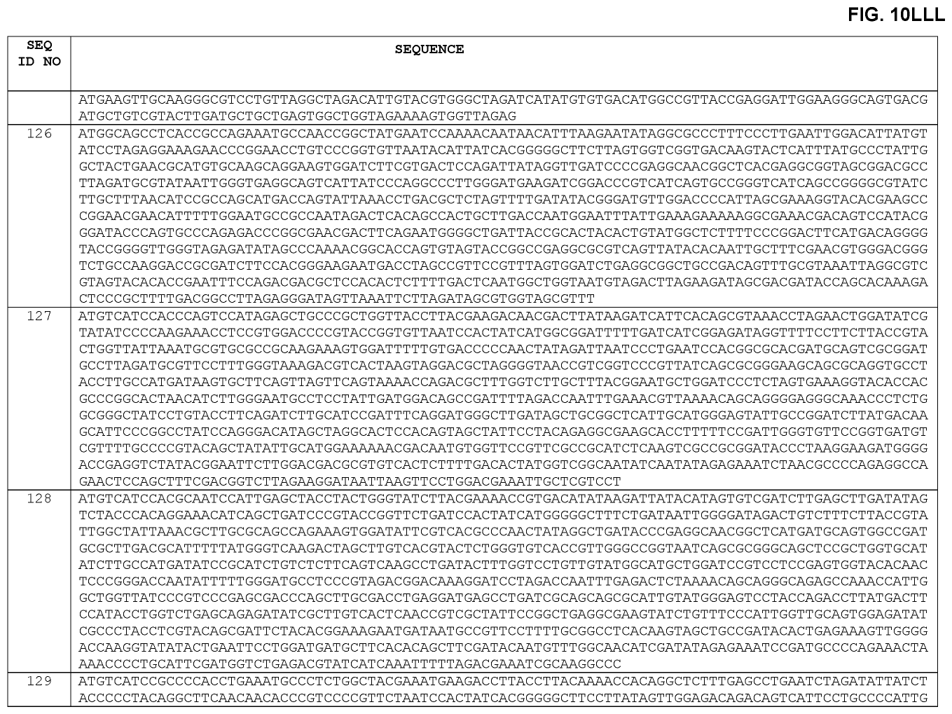

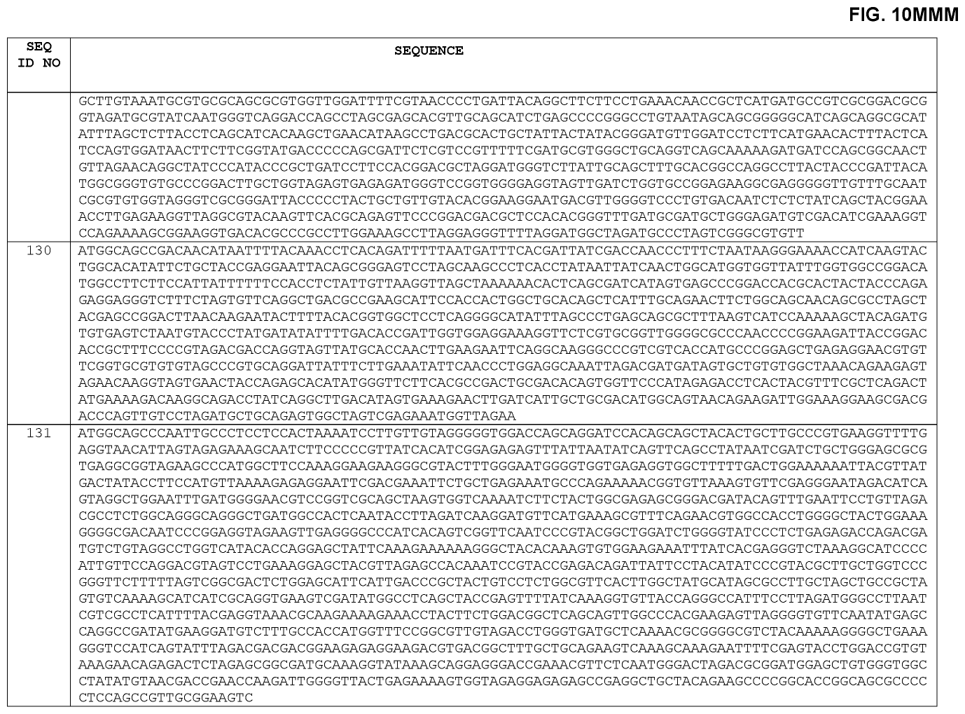

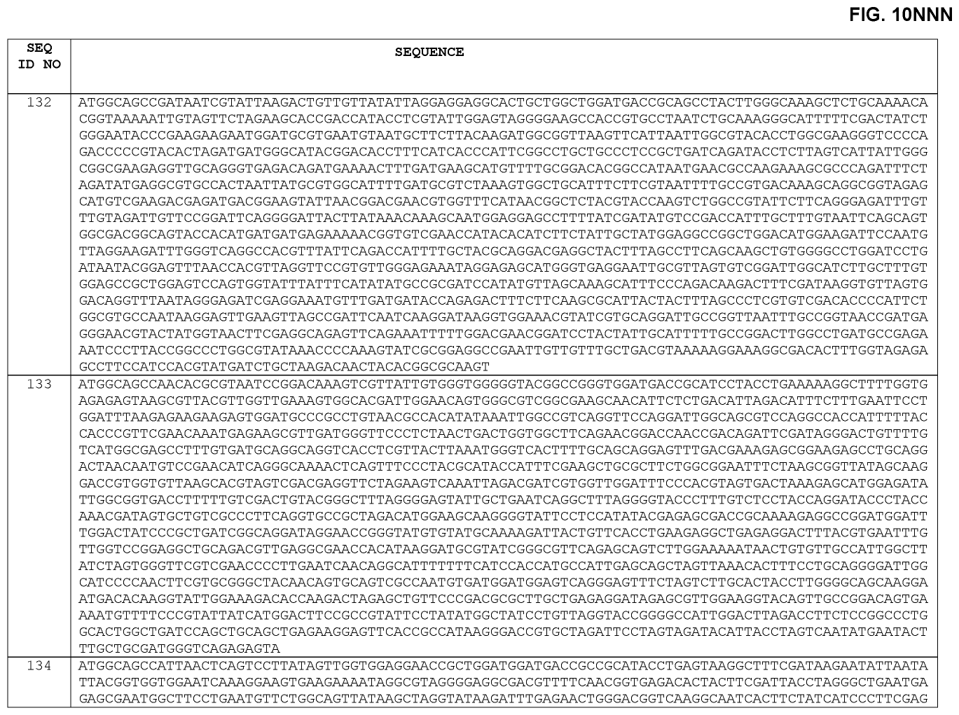

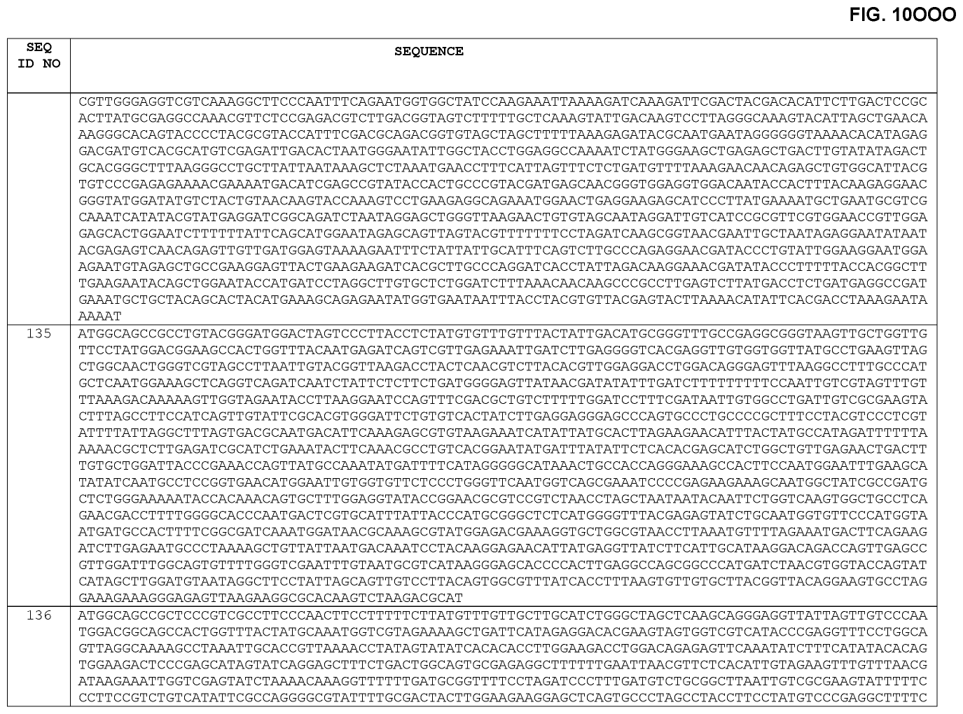

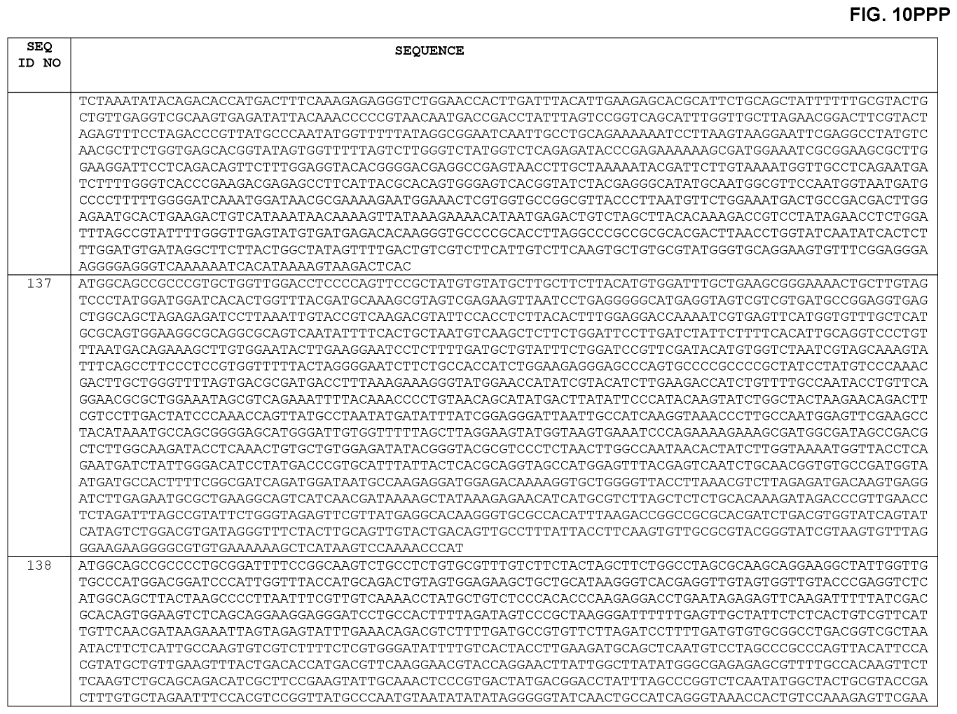

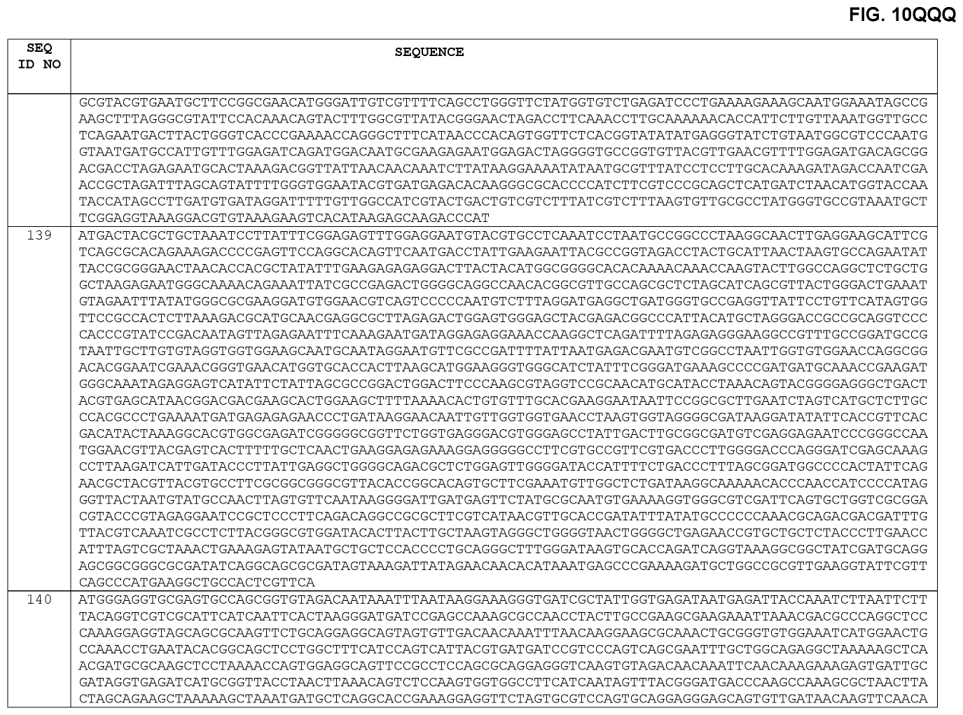

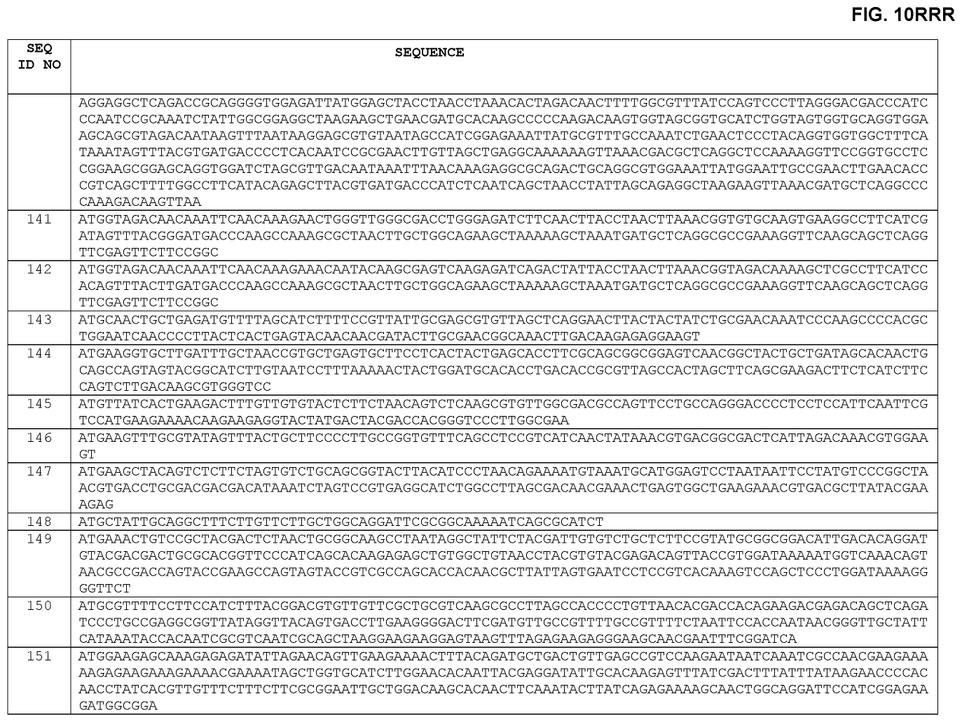

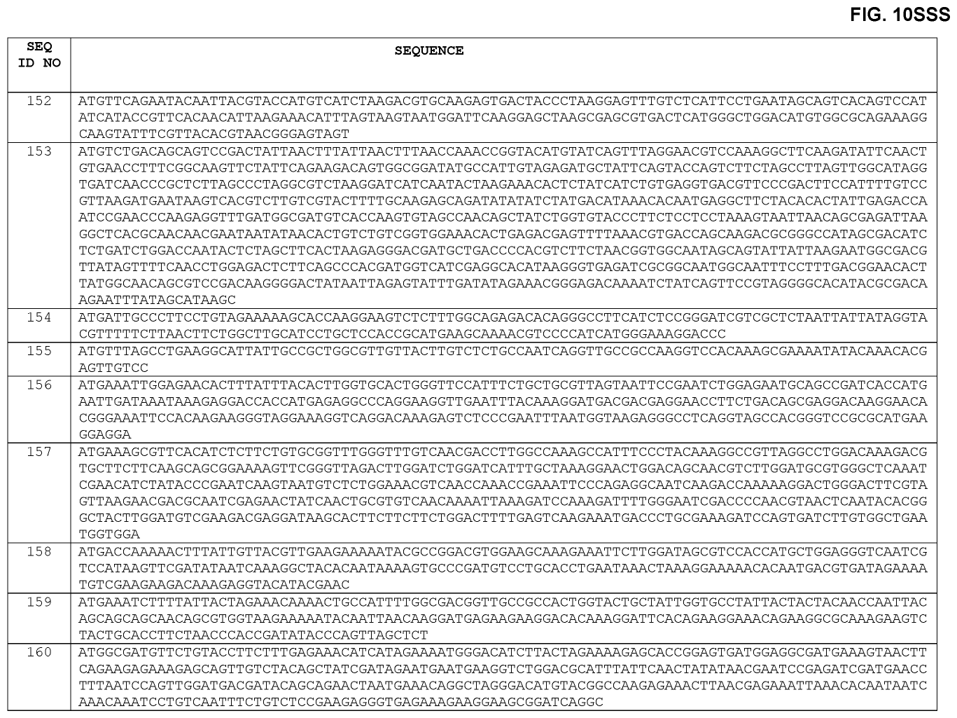

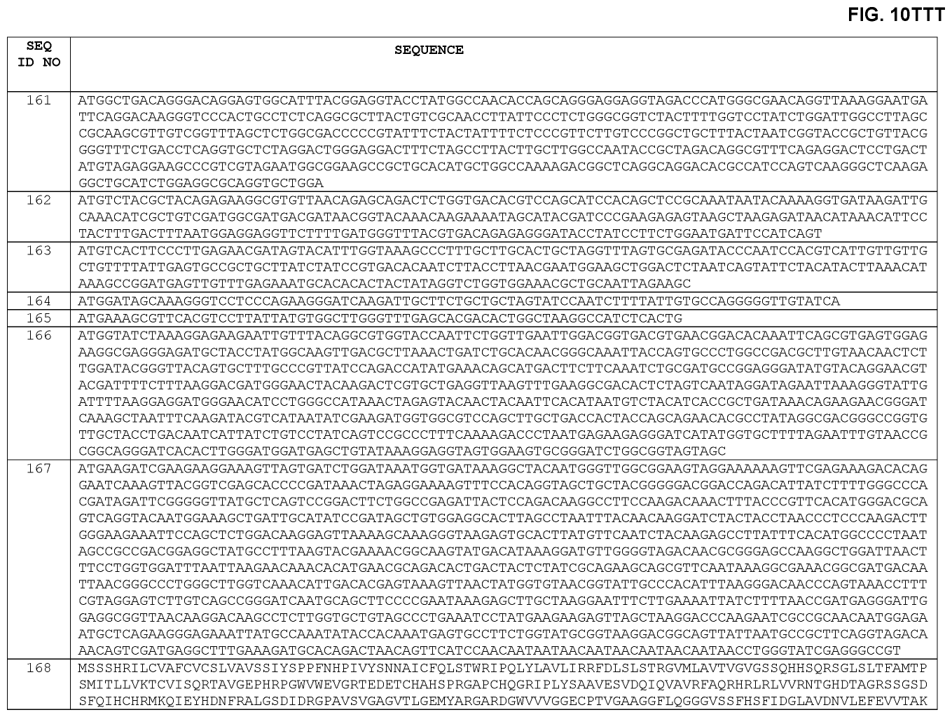

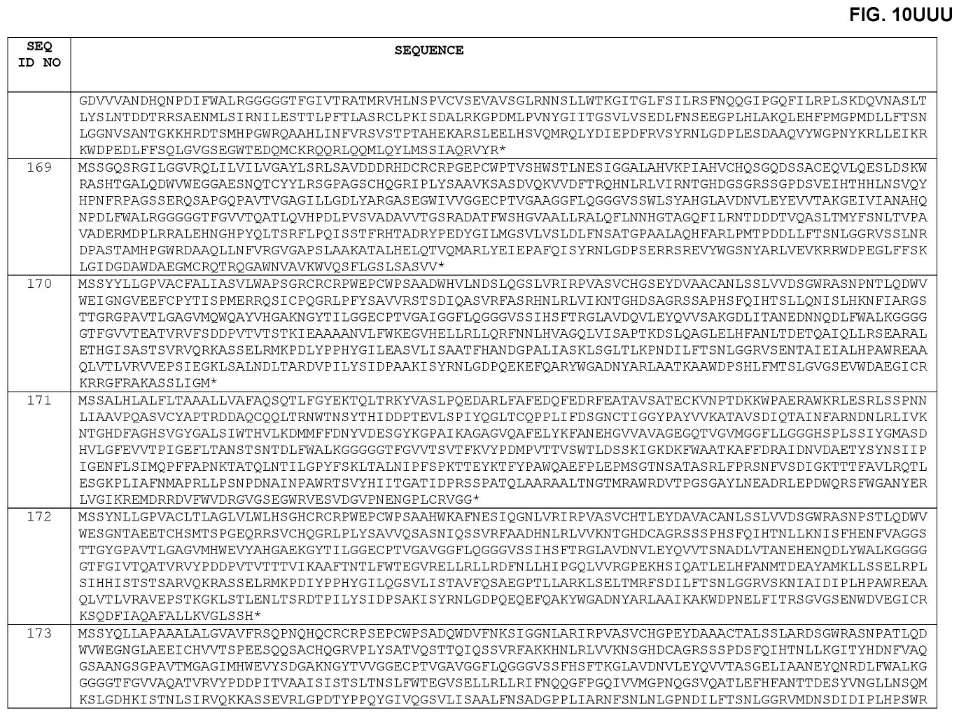

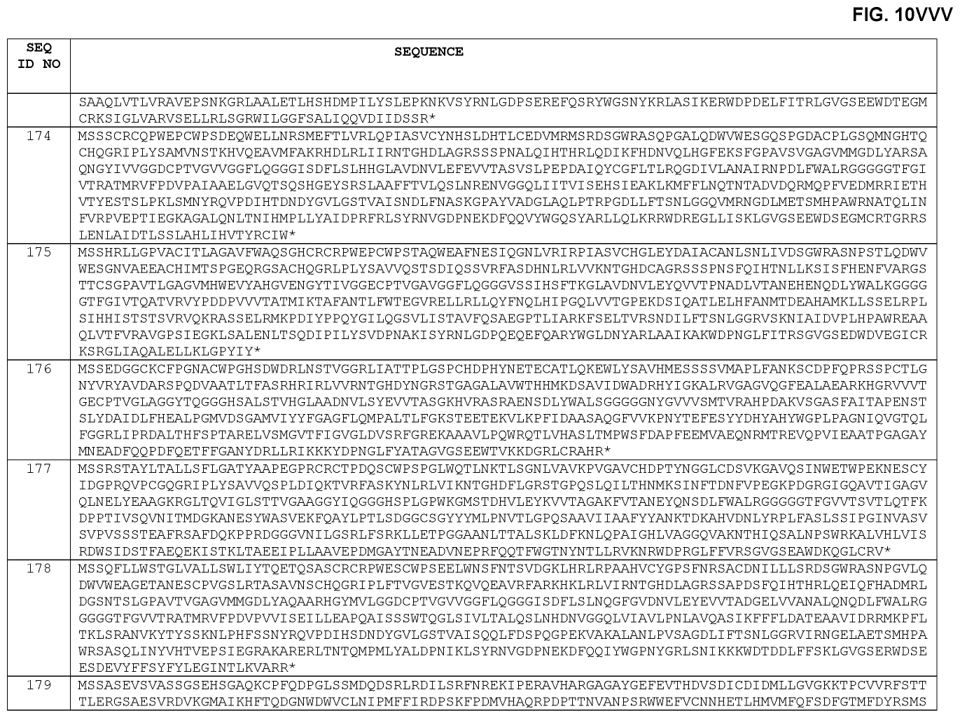

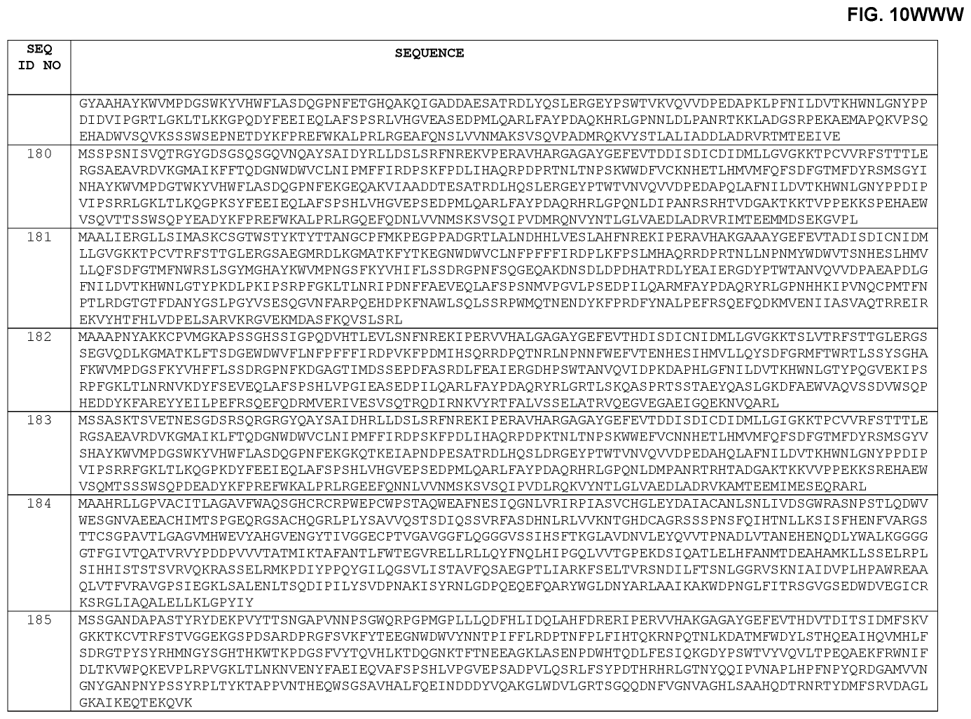

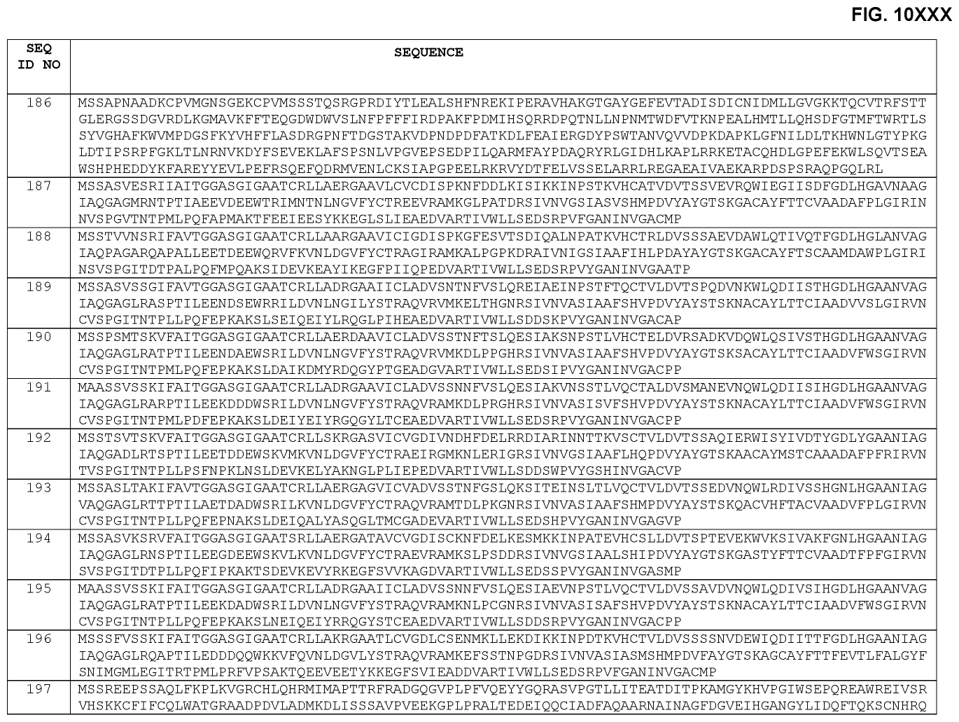

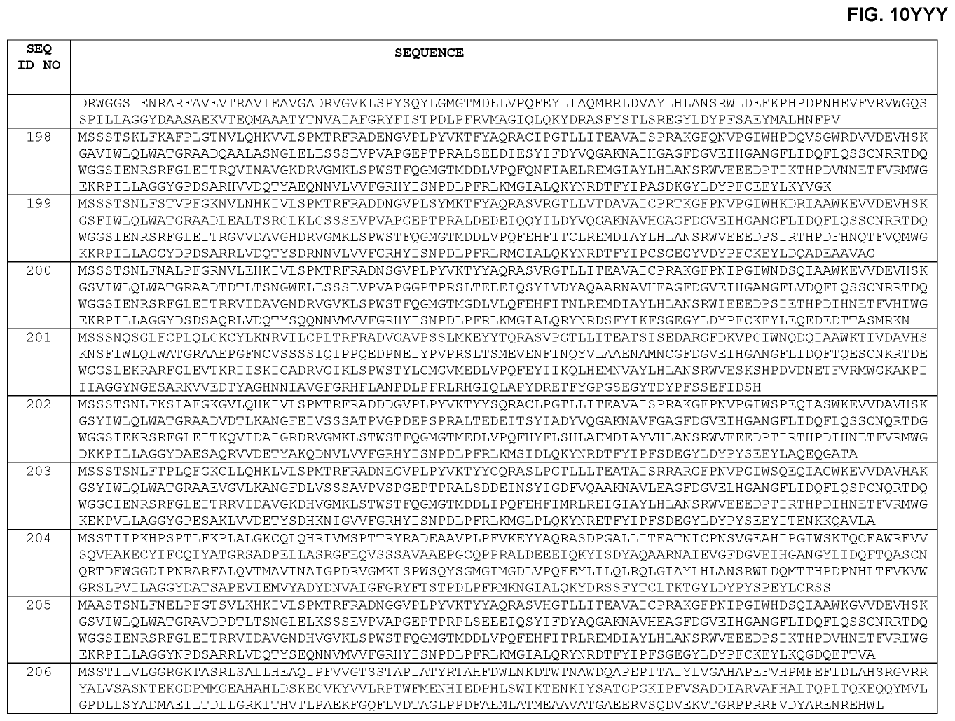

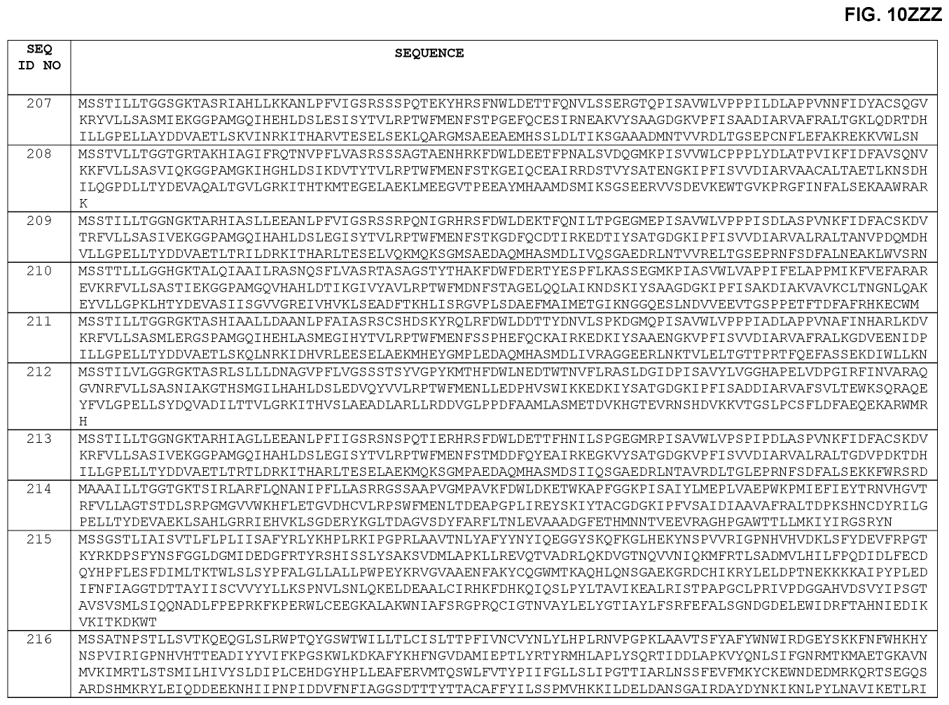

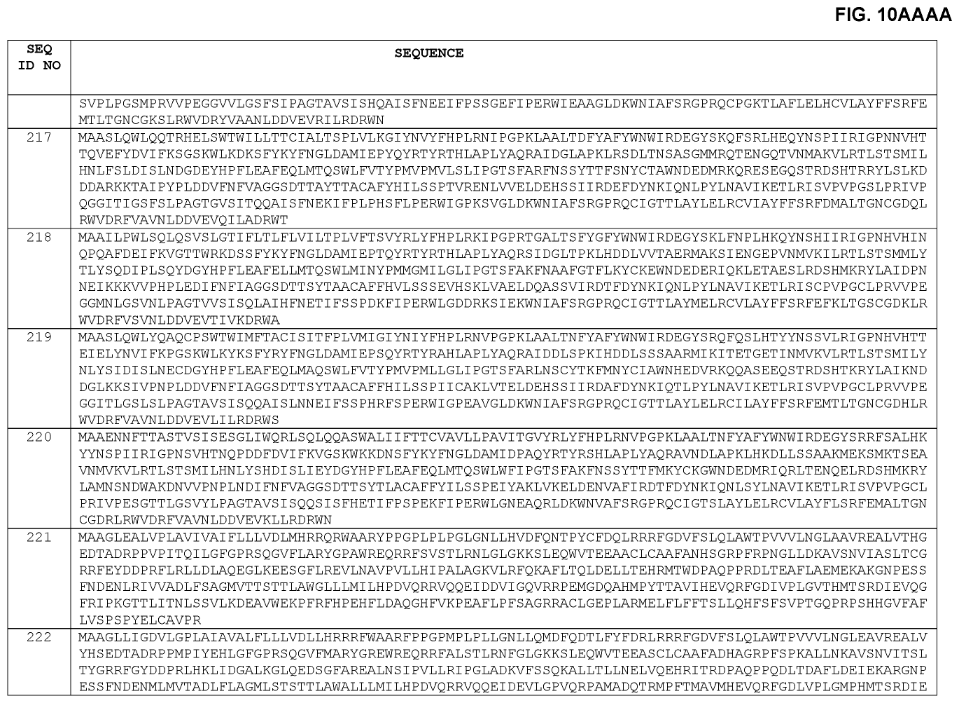

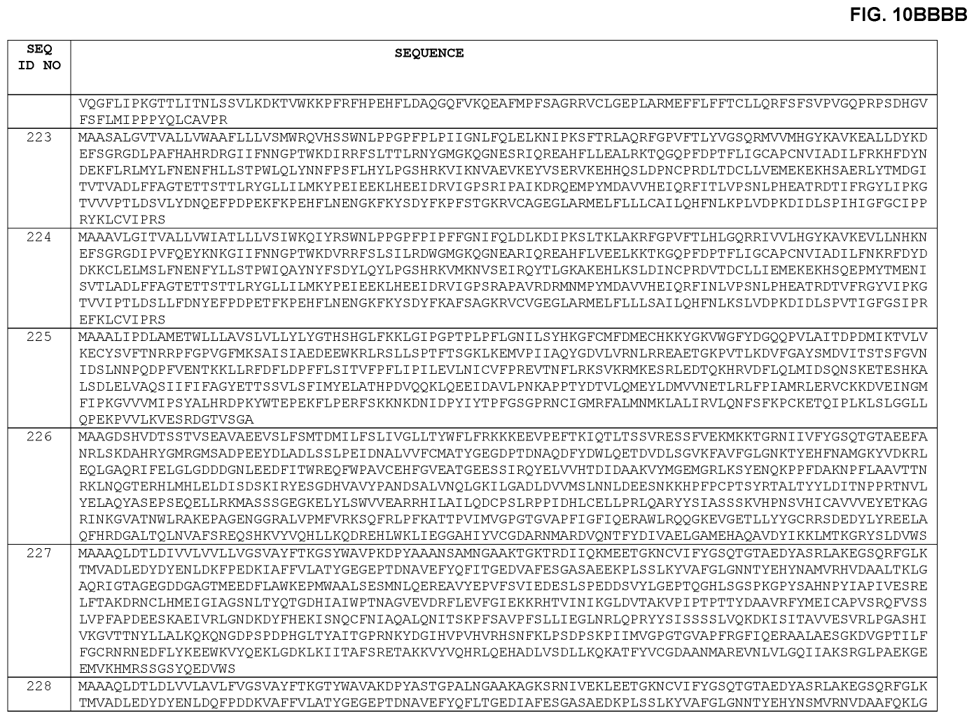

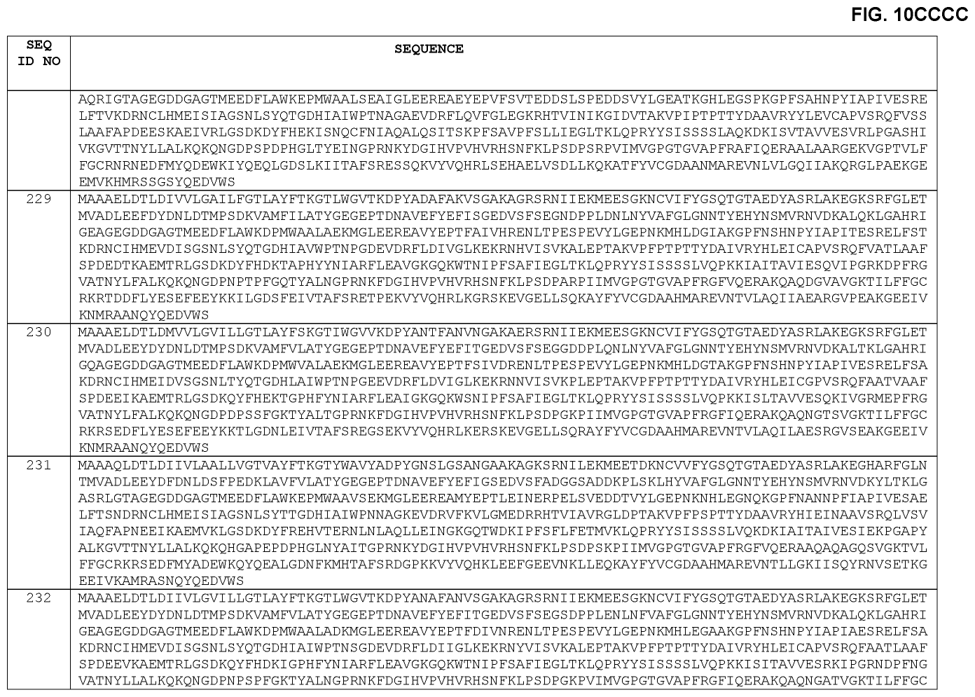

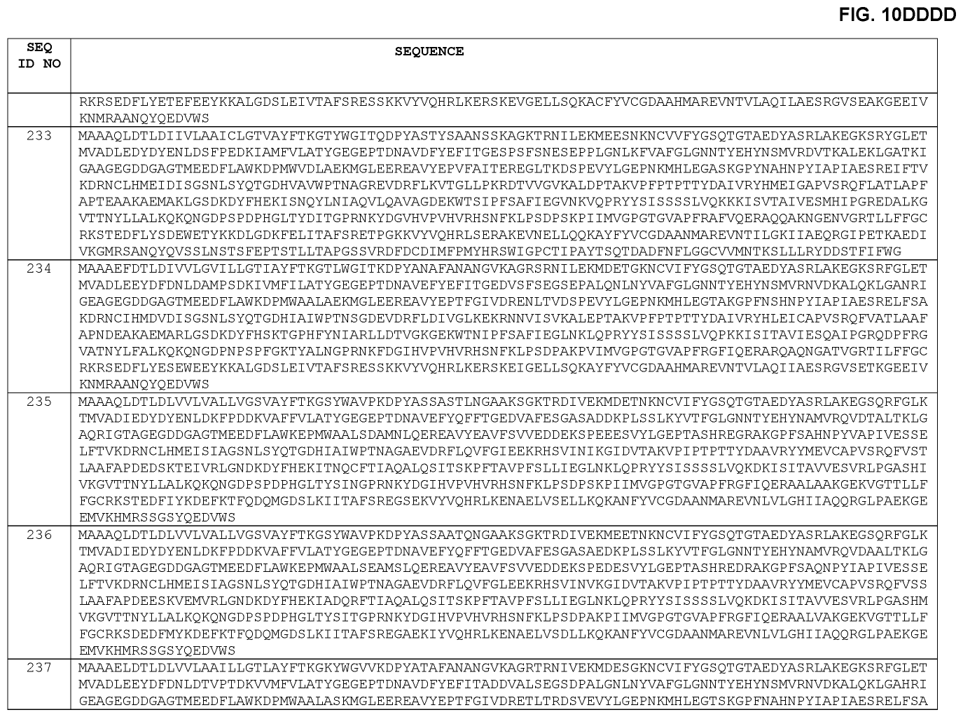

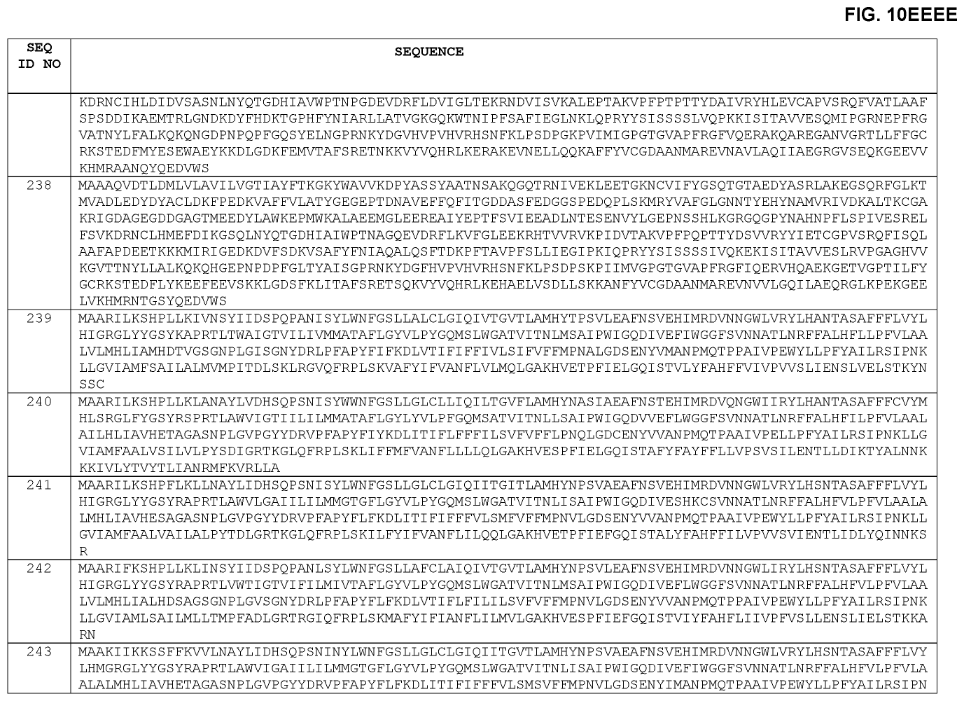

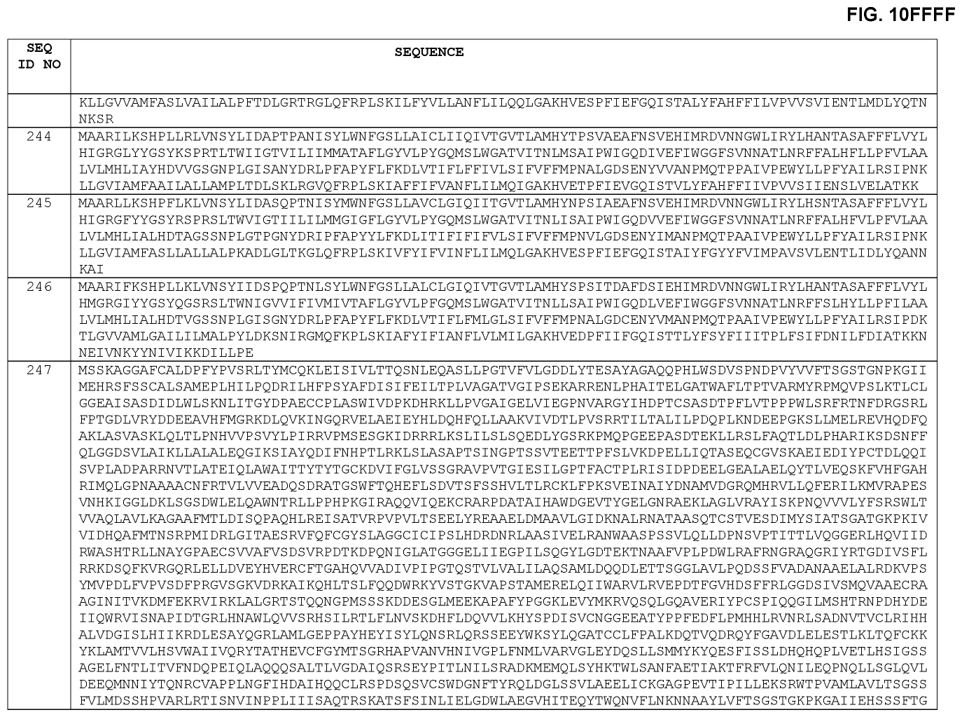

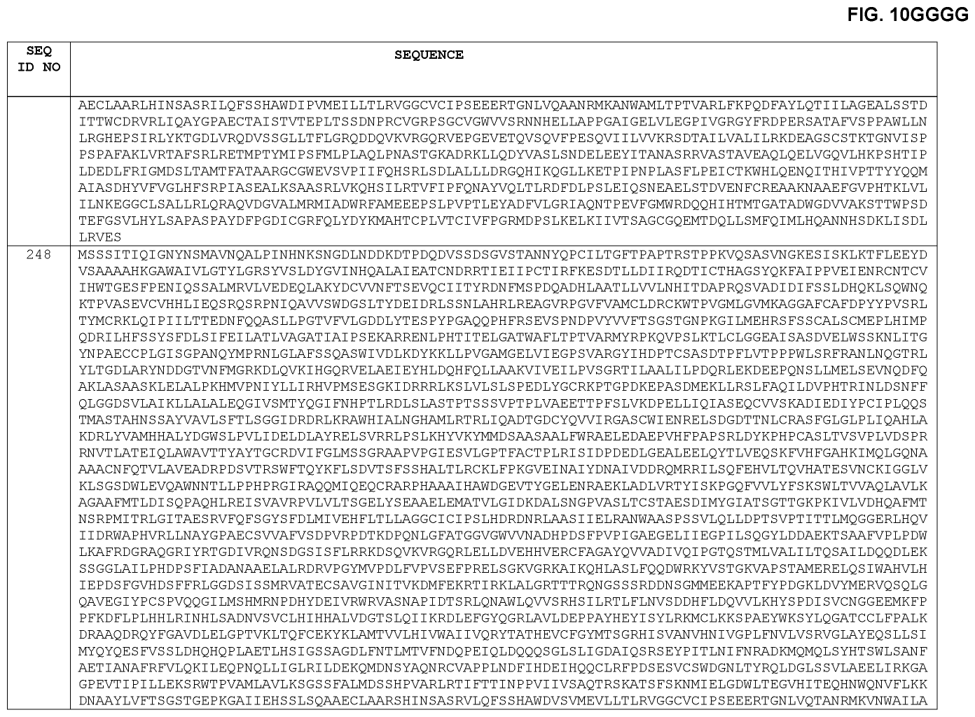

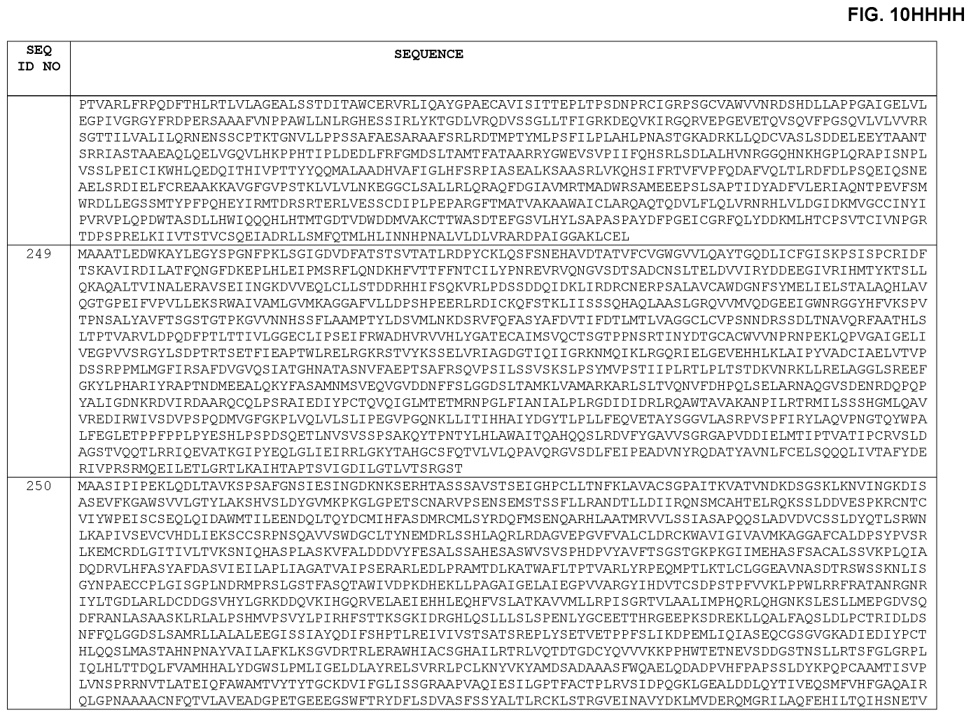

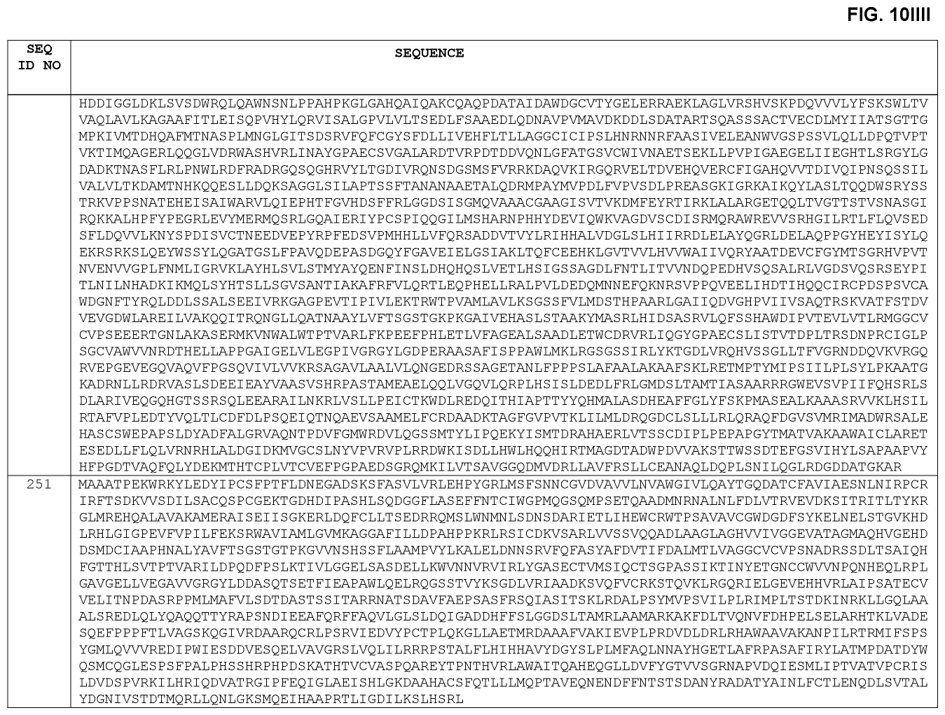

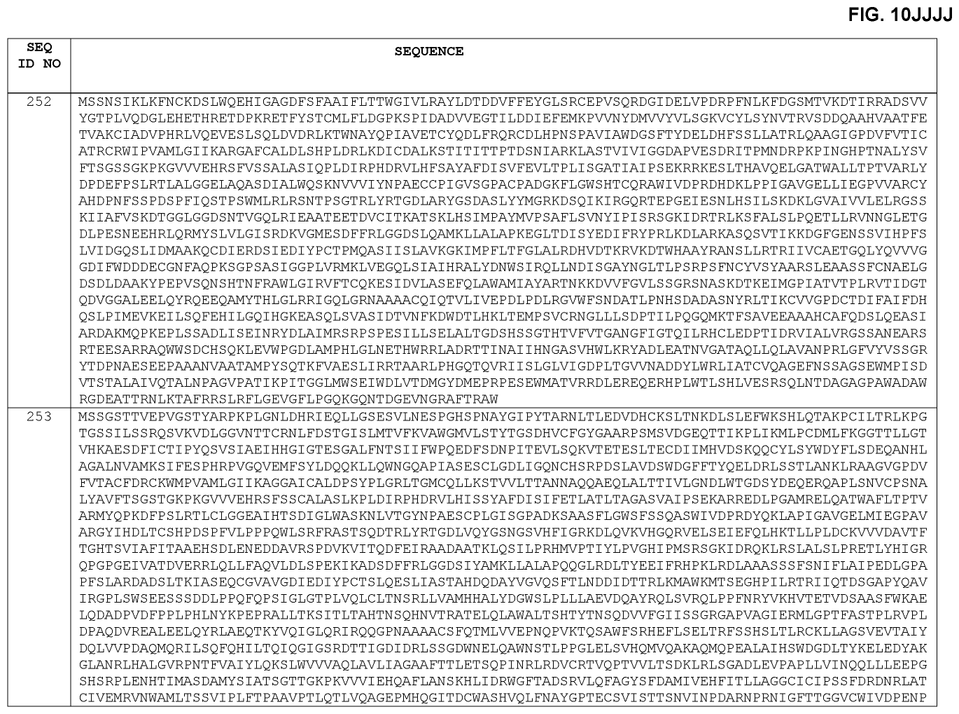

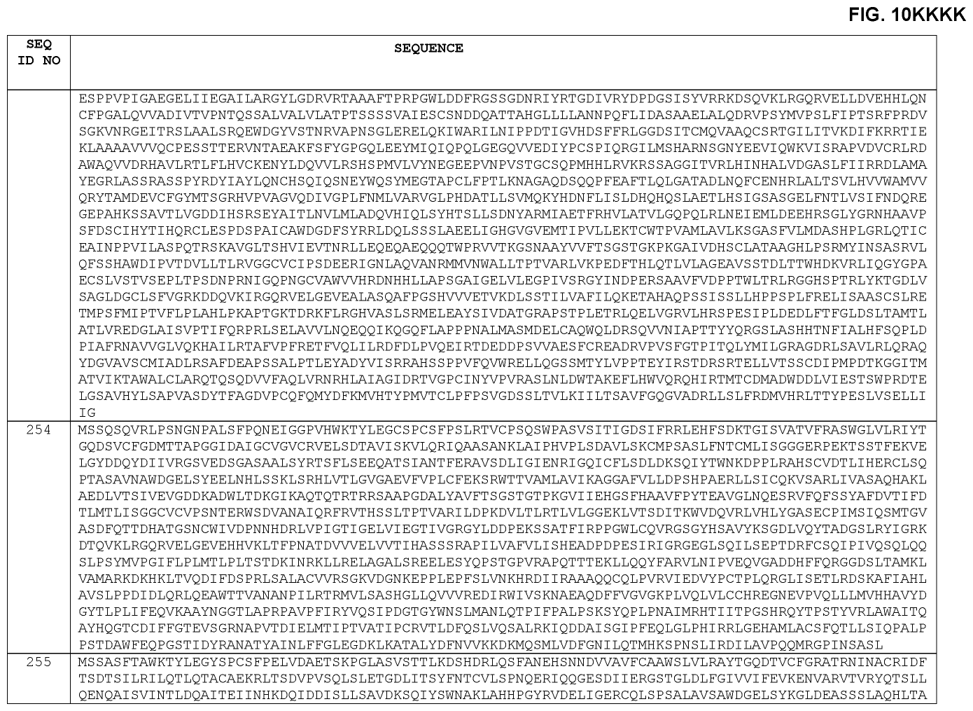

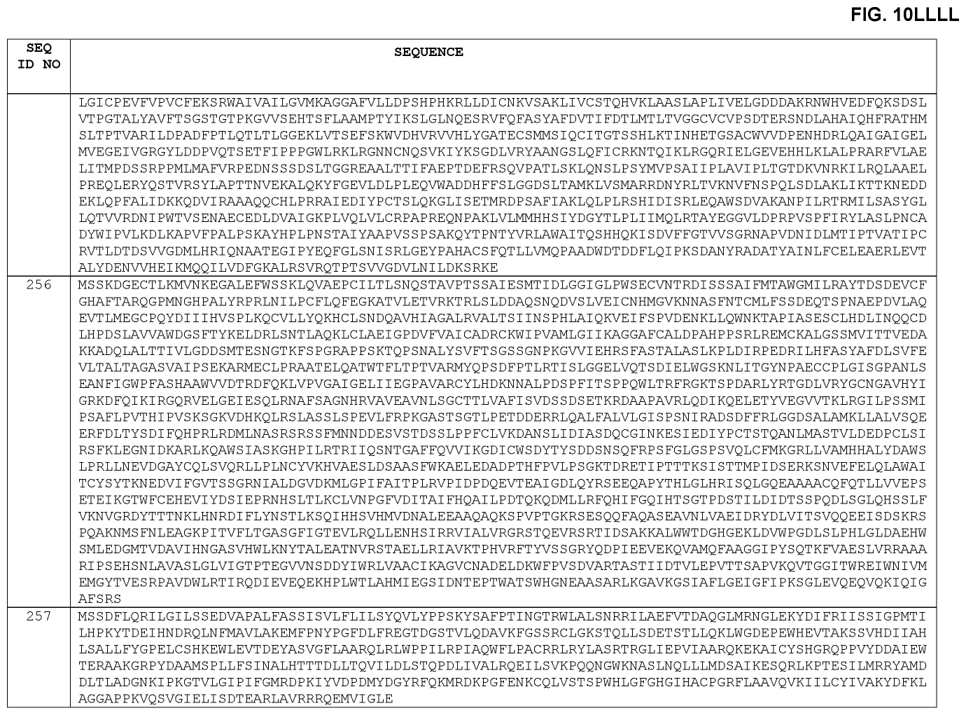

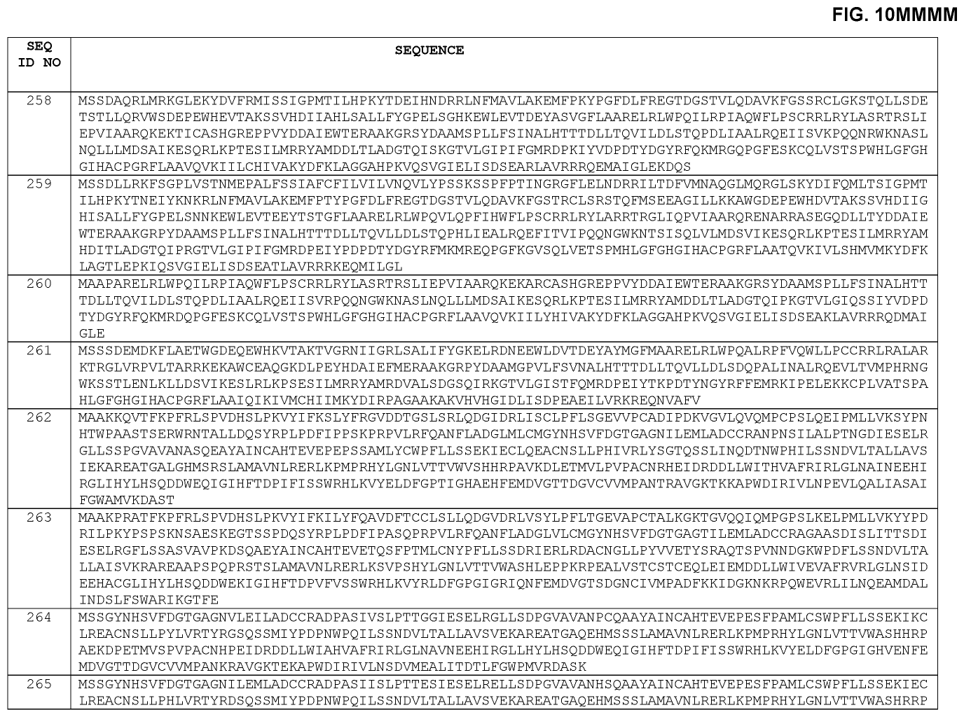

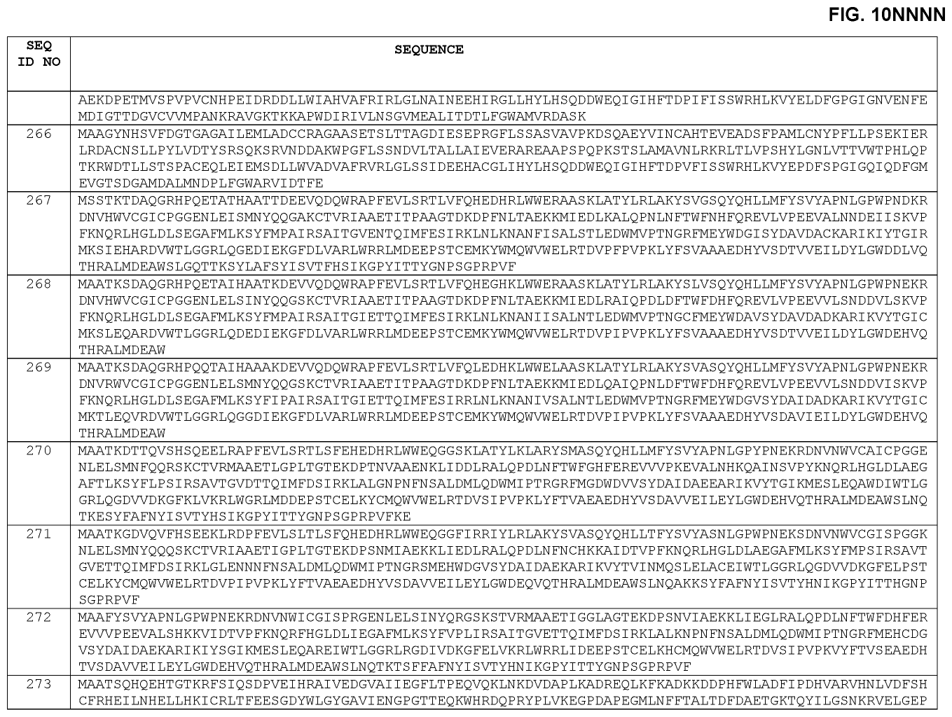

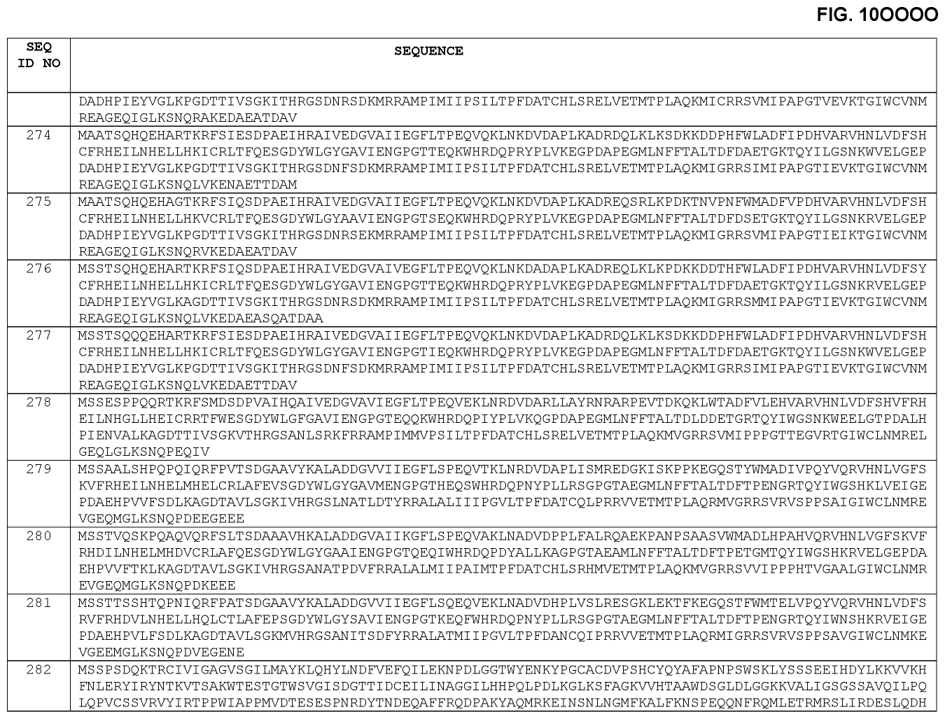

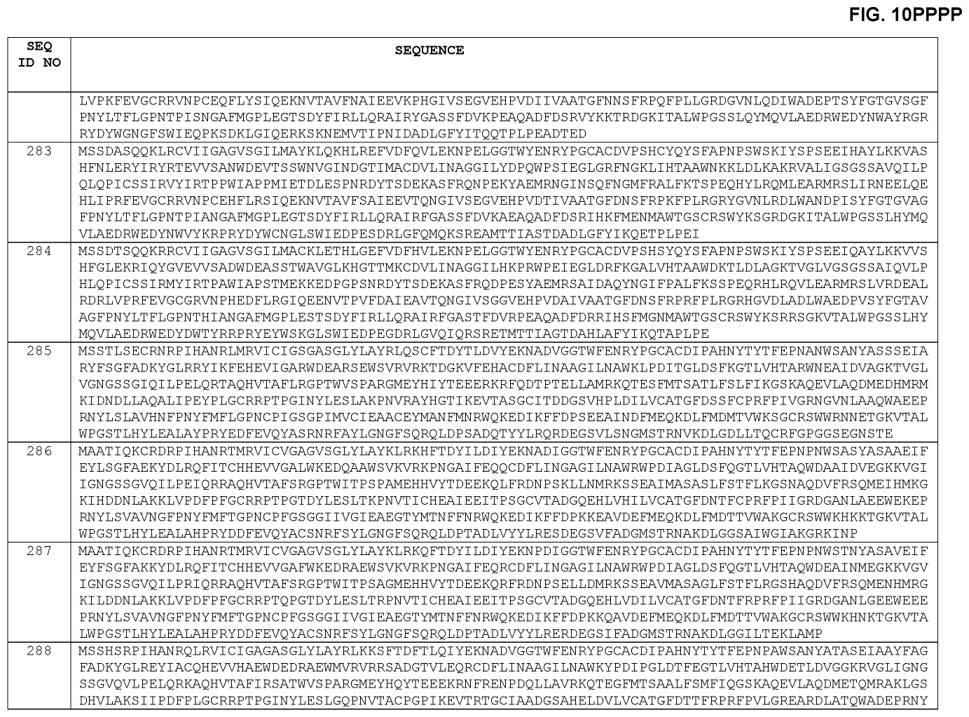

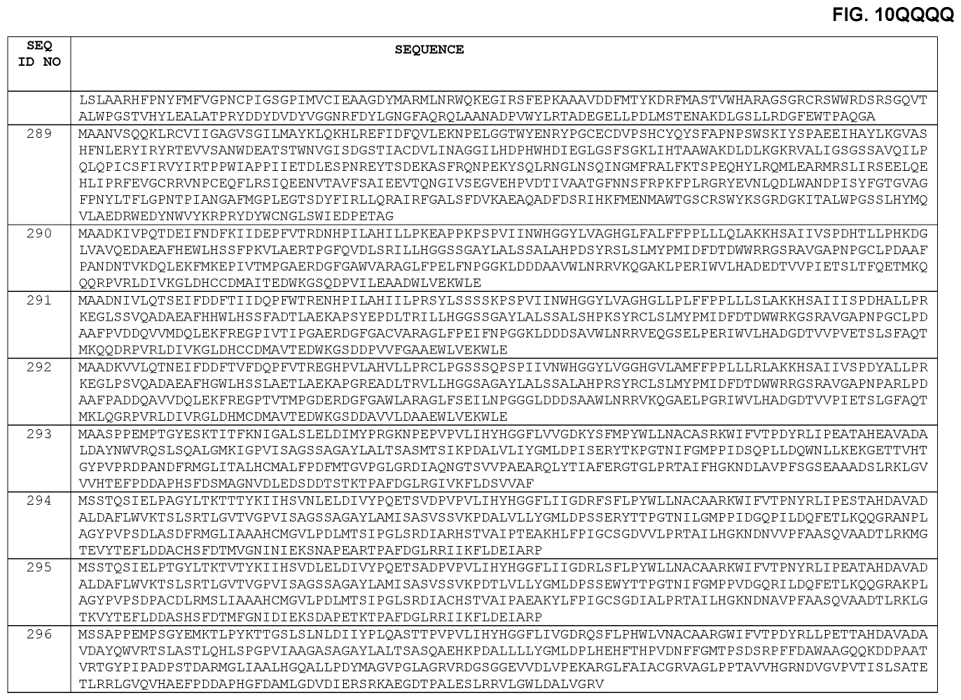

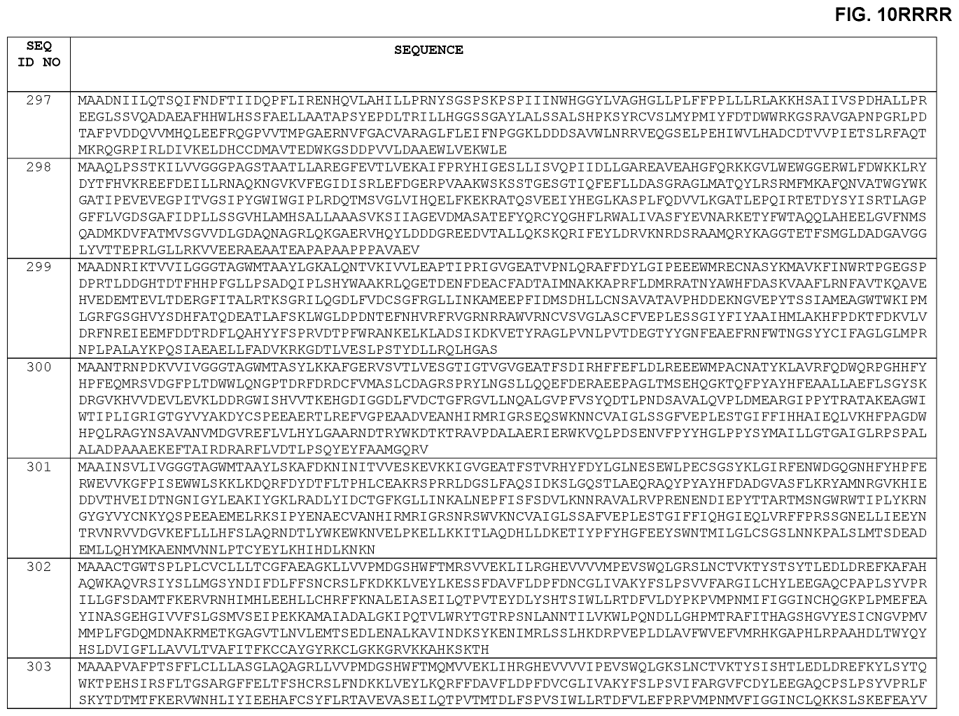

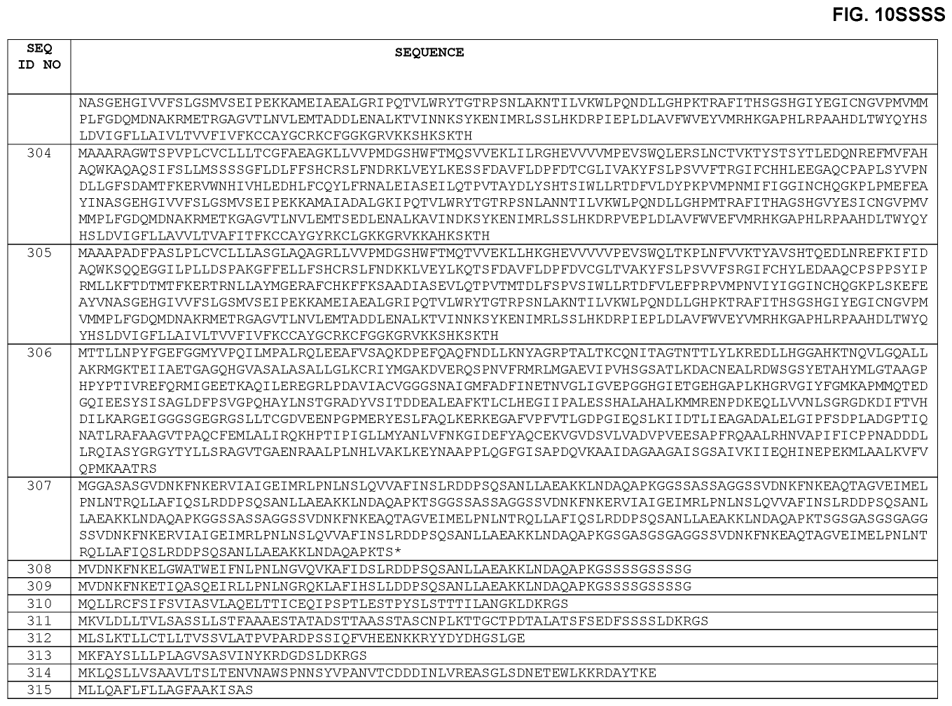

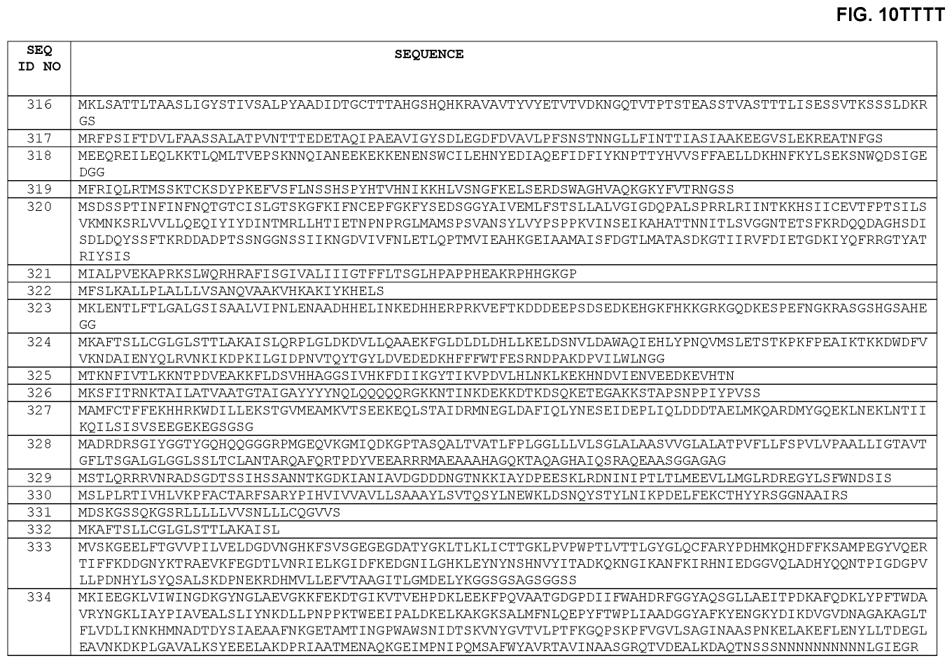

Oxidation of chanoclavine to chanoclavine aldehyde is catalyzed by EASD, followed by ring formation of chanoclavine aldehyde to agroclavine or festuclavine, catalyzed by EASG. (See FIG. 2 Panel A) This series of steps may function best if enzymes are co-localized and expressed together. Contribution of other enzymes, such as EASA may facilitate high levels of production of agroclavine or festuclavine. (See FIG. 8 and FIG. 9)

As described herein, several combinations of EASD/EASG EASD/EASA, and EASA/EASG enzymes are effective for the synthesis of agroclavine in chanoclavine-producing recombinant strains. The EASD and EASG enzymes may, but need not, originate from the same organism. In addition, as described herein, EASA enzymes from certain organisms are able to take the role of the EASG enzymes (i.e., can provide equivalent activity for ergoline synthesis). In such strains, agroclavine is synthesized by an EASA/EASG enzyme pair rather than a EASD/EASG enzyme pair.

This activity stands in contrast to any previously described biosynthesis of agroclavine (e.g., Wong, 2022), as the polynucleotides sequences, amino acid sequences, and recombinant cells comprising them can generate agroclavine with both EASA/EASG and EASD/EASG enzyme pairs. The conversion of chanoclavine to agroclavine is thought to proceed through the intermediate chanoclavine aldehyde. However, in the illustrative embodiments described in the Examples below, no intermediates between chanoclavine and agroclavine were detected, indicating all intermediate was consumed.

Isolation and detection of ergoline products from recombinant cell, lysate, or co-culture fermentation broth can be performed by solvent extraction, filtration, and analytical methods including high performance liquid chromatography (HPLC), liquid chromatography mass spectrometry (LC-MS), gas chromatography (GC), gas chromatography mass spectrometry (GC-MS), and/or nuclear magnetic resonance (NMR) methods, as known to those skilled in the art. In some embodiments, recombinant host strains expressing the ergoline biosynthesis pathway can be mixed with a solvent such as acetonitrile, ethanol, isopropanol, methanol, ethyl acetate, acetone, water, and mixtures thereof. The solvent can further include one or more additives such as, for example, formic acid (FA), ammonium carbonate, or trifluoroacetic acid (TFA) in amounts ranging from 0.01-20% volume to volume. Such solvent mixtures can be applied to whole broth, cell pellets, or fermentation supernatant in combination with centrifugation, vortexing, bead-beating, homogenization, filtration (such as tangential flow filtration (TFF)), and/or shaking and incubation, at varying temperatures (e.g., from 4-100° C.) for various periods of time (e.g., from 1-120 minutes). Such extractions can then filtered for running on analytical equipment, such as dead-end filtration through a 0.1-0.22 micron filter of polytetrafluoroethylene (PTFE), polyvinylidene fluoride (PVDF), nylon, or polyethersulfone (PES) to remove unwanted debris from sensitive analytical procedures.

Generating Lysergic Acid from the Conversion of Agroclavine to Elymoclavine and Elymoclavine

Some of the downstream steps in the ergoline pathway are catalyzed by CYP P450s with accompanying cytochrome reductases (CPR), including the successive oxidations of agroclavine into elymoclavine, paspalic acid, and then isomerization into lysergic acid (See FIG. 2 Panel B). Enzymes may be used alone, or co-expressed with other cytochrome P450s to maximize flux through. CloA is the P450 known to catalyze this multistep oxidation. It requires a CPR to supply reducing equivalents. The oxidation reaction will be supported by an abundance of NADPH in the cell. The requirement for NADPH may be supported by the over expression of NADPH producing enzymes such as ZWF1.

Non-P450 monooxygenases may help catalyze oxidation steps between agroclavine and lysergic acid. Monooxygenases may be sourced from fungi known to produce ergolines, such as those in the family Claviceptacae, Trichocomaceae, Aspergillacea, Arthroderma. They may also be sourced from bacteria and plants that contain a homologous cluster of biosynthetic enzymes that contains an indole prenyl transferase N-methyltransferase and other members of the ergoline pathway.

Non P450 monooxygenases may also catalyze the relevant modification. Monooxygenases may include examples (see, e.g., Torres Pazmino, 2010) such as:
Fe2/alphaketoglutarate dependent oxygenases
Flavin-dependent monooxygenases are not heme dependent. An example is Cyclohexanone monooxygenase
Copper-dependent monooxygenases
Bacterial polysaccharide monooxygenases,
Non-heme iron-dependent monooxygenases
Pterin-dependent monooxygenases
Diiron hydroxylases
Other cofactor-dependent monooxygenases
Cofactor-independent monooxygenases

To improve production of active CYP P450 proteins, N-terminal sequences may be removed and replaced with a sequence that targets the enzyme to the membranes of organelles, such as the ER, golgi, mitochondria, peroxisome, and/or vacuole.

The ratio of CPR to CYP, along with the co-expression of cytochrome b5 (CYB), may be adjusted to give optimal enzymatic activity in the recombinant ergoline biosynthesis pathway.

Iron, in oxidized, reduced, or chelated states, including heme, leghemoglobin, and/or agricultural products containing leghemoglobin, may be added to the culture media to stimulate the activity of the P450 enzyme.

In some embodiments, festuclavine is converted to dihydro lysergic acid, in a similar manner to the agroclavine conversion to lysergic acid described above. Festuclavine undergoes successive oxidations reactions by P450 enzymes such as CloA to yield dihydro lysergic acid (FIG. 2 Panel C).

### Peptide Synthase Enzymes for Production of Ergopeptines

Peptide synthases, called lysergyl peptidyl synthetases (LPS) add an amino acid moiety to lysergic acid to produce ergopeptines. LPSs use proline to form a tripeptide with two other amino acids (see Table 2 below) that couples to the ergoline moiety to form the lactam. These enzymes may need to be overexpressed as pairs to be functional. LPS enzymes may be sourced from fungi known to produce ergolines, such as those in the family Claviceptacae, Trichocomaceae, Aspergillacea, Arthroderma, Neotyphodiums. They may also be sourced from bacteria and plants that contain a homologous cluster of biosynthetic enzymes that contains an indole prenyltransferase, N-methyltransferase, and other members of the ergoline pathway.

Amino acid addition, particularly of proline will help stimulate ergopeptine catalysis. Amino acid supplement may be in the form of purified amino acids or may be derived from blended or raw sources including fish meal, fish waste, soy, corn, pea or wheat protein extracts, or dairy byproducts (as generally known in the art).

Other ergoline derivatives can be generated and include, for example, dihydroergocryptine, cabergoline, lisuride, bromocriptine, lergotrile and pergolide. Additional ergoline derivative compounds are listed in Tables 1 and 2 below.

TABLE 4
Representative peptide-like ergots

| Group | Compound name |
|---|---|
| Ergotamine | Ergotamine |
| | Ergotaminine |
| | Ergosine |
| Ergotoxine | Ergosinine |
| | Ergocristine |
| | Ergocristinine |
| Ergotoxine | Ergocryptine |
| | Ergocryptinine |
| | Ergocryptine |
| | Ergocryptinine |

TABLE 5
Examples of ergopeptines and their
amino acid (AA1-3) composition

| Compound | AA1 | AA2 | AA3 |
|---|---|---|---|
| Ergotamine | L-alanine | L-phenylalanine | L-proline |
| Ergosine | L-alanine | L-leucine | L-proline |
| Ergocornine | L-valine | L-valine | L-proline |
| Ergocryptine | L-valine | L-leucine | L-proline |
| Ergocristine | L-valine | L-phenylalanine | L-proline |
| Ergovaline | L-alanine | L-valine | L-proline |
| Ergobalansine | L-alanine | L-leucine | L-alanine |

Conversion of Lysergic Acid and Peptide-Type Ergots into Lysergic Acid Amides

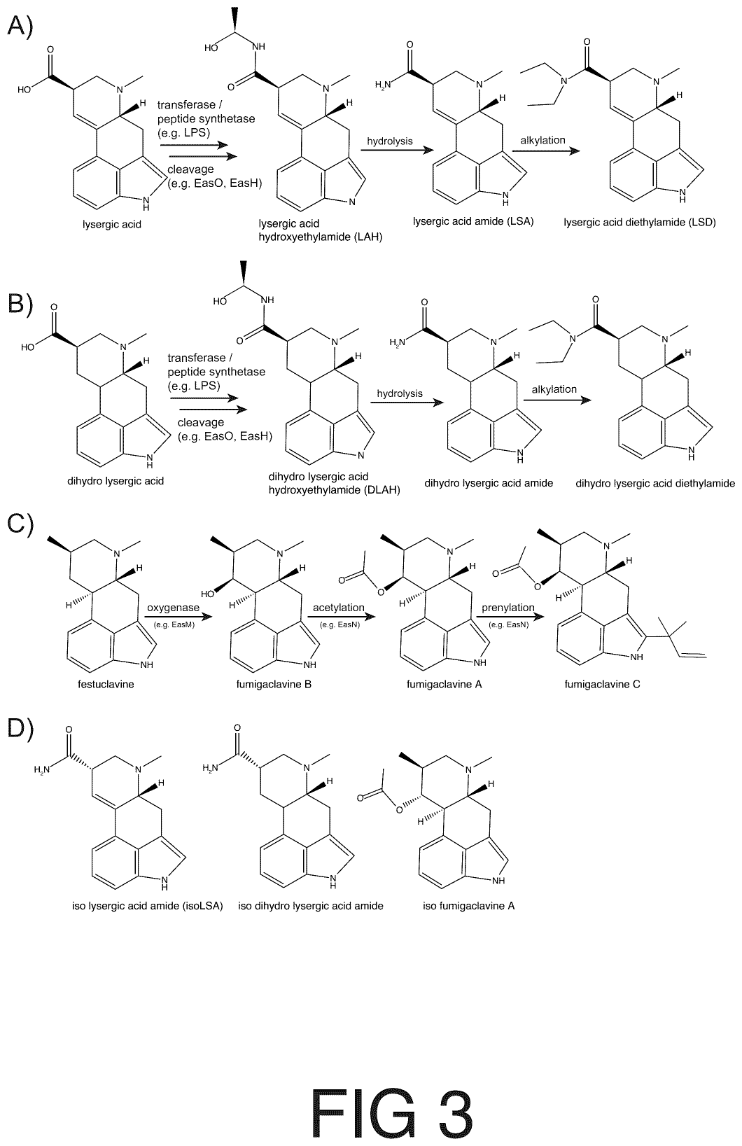

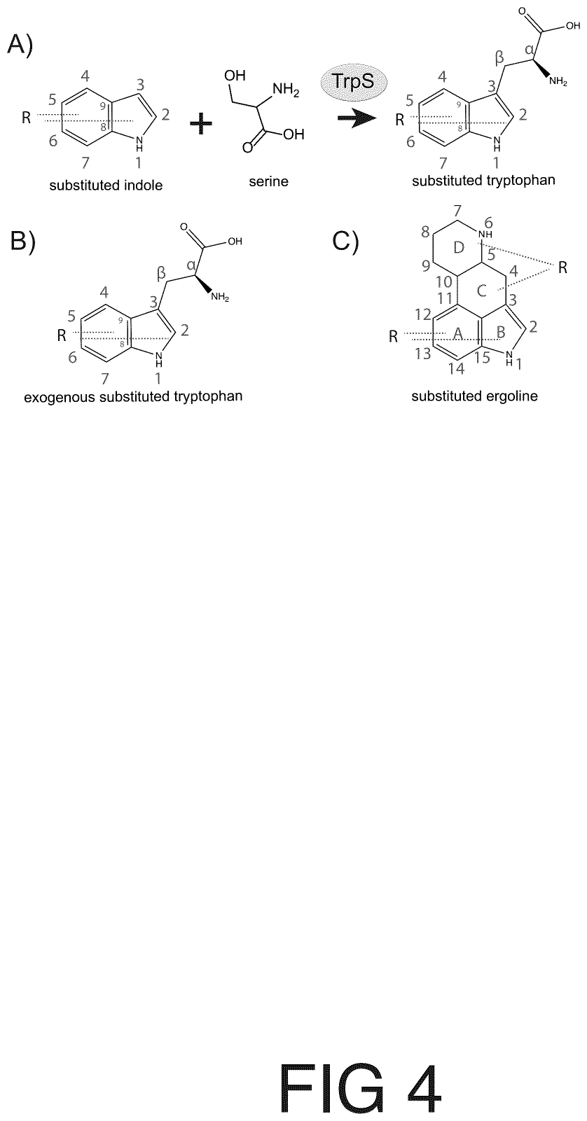

Lysergic acid can be further derivatized into amide analogs, including compounds containing primary, secondary, and tertiary amines, such diethylamide. In some embodiments, lysergic acid is processed by lysergyl peptide synthetase (LPS), followed by hydrolysis and/or amidase activity (e.g. EASO, EASH) to yield lysergic acid hydroxyethylamide (LAH), which undergoes hydrolysis to form lysergic acid amide (LSA), which can be further alkylated to yield lysergic acid diethylamide (LSD) (FIG. 3 Panel A). Similarly, dihydro lysergic acid, can yield dihydro lysergic acid hydroxyethylamide (DLAH) and dihydro lysergic acid amide (DLSA), which can be further derivatized (FIG. 4 Panel B). Festuclavine is also a branchpoint from the dihydro lysergic acid and dihydro lysergic acid amide pathway. Festuclavine can undergo oxidations to yield fumigaclavine B, followed by acetylation to yield fumigaclavine A, which can then be prenylated to yield fumgaclavine C (FIG. 3, Panel C). Stereoisomers of these compounds can also be generated (FIG. 3, Panel D).

### Fermentation Conditions

Fermentation conditions may strongly influence the success of the biosynthetic effort. Temperatures other than the standard microbial 30 degrees Celsius may help with protein expression and yield of functional enzyme for steps in ergoline biosynthesis and metabolism. 15-37 degrees Celsius is an acceptable temperature range. Redox will strongly influence the efficiency of some of the reactions. Addition of oxidants such as hydrogen peroxide, antioxidants such as ascorbic acid, BHA, BHT or carotenoids and reductants such as BME, DTT or TCEP to the culture media may help some enzymatic reactions proceed. The enzymes in the pathway may be co-expressed or may require stepwise expression.

Fermentation media with additives may help improve functional ergoline enzyme expression, such as media or a co-feed containing glycerol, trehalose, trimethylamine N-oxide (TMAO), sorbitol, mannitol, malt syrup, molasses, corn steep liquor, lactobionic acid, gluconolactone, mannose, fucose, xylose, levulose, sucrose, galactose, raffinose, cellulose, lactose, maltose, arginine, proline, betaine, and/or glycine, likely by helping to stabilize and assist correct folding of newly expressed proteins.

### Co-Culture Methods

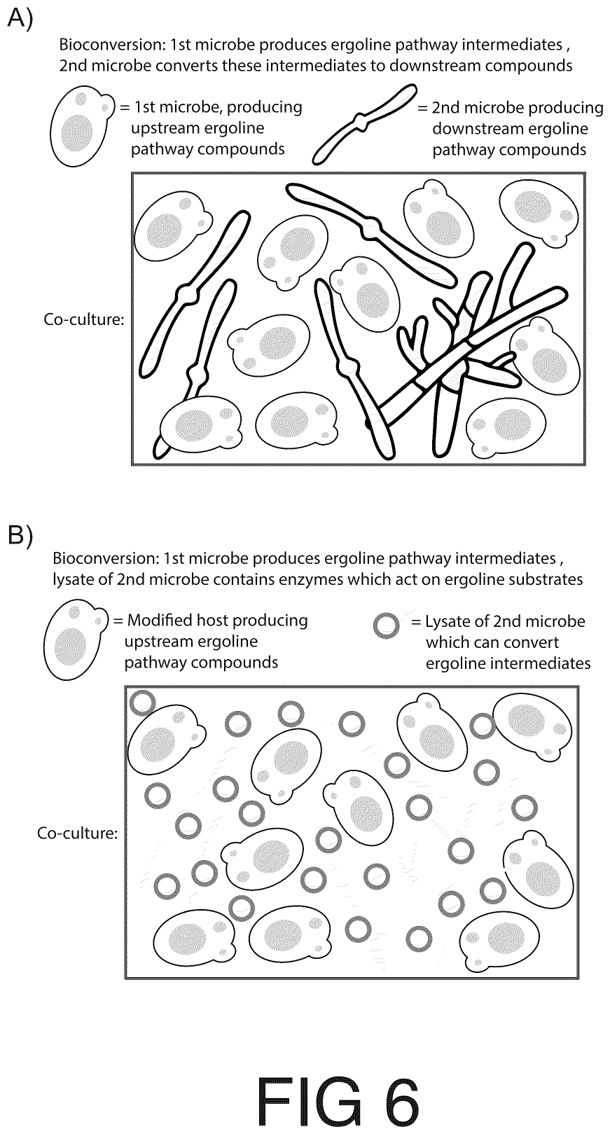

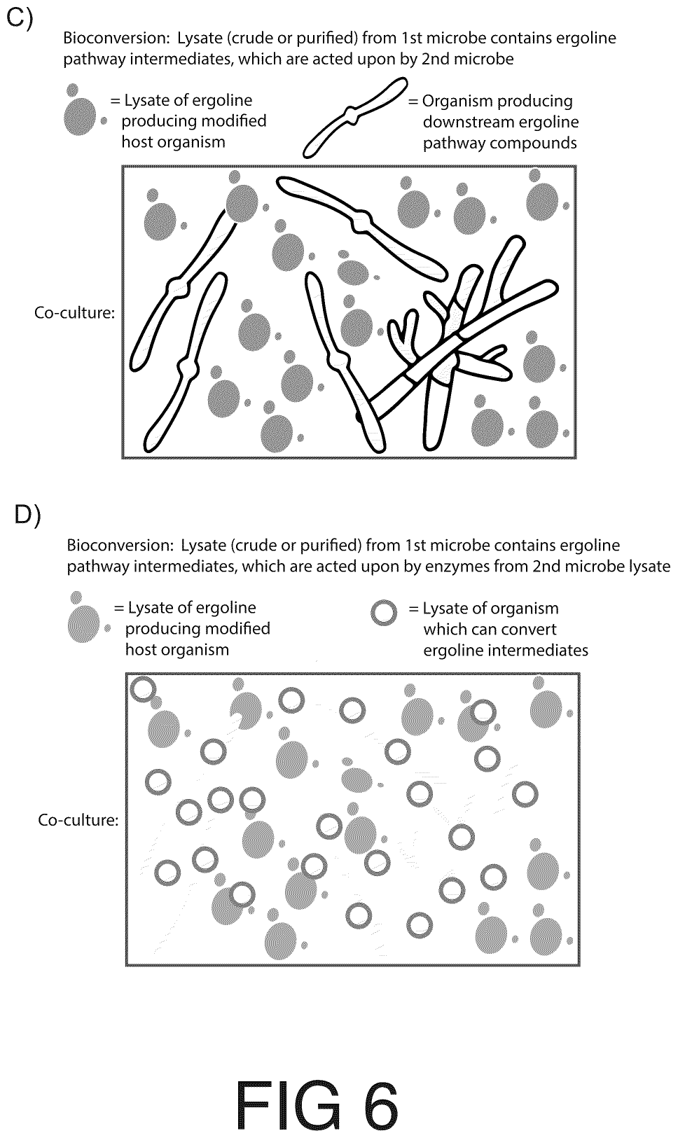

Enzymes comprising the ergoline pathway may all be co-expressed in a single recombinant host cell to produce ergolines. Alternatively, some enzymes in the pathway may be expressed in a single recombinant host cell while others are expressed in a second host cell. The second host cell may be recombinant or may express the desired enzymes without any modification. The two host cells may or may not be the same species. (See, e.g., FIG. 6A). In some embodiments, the co-culture methods can comprise one or more bioconversions, as discussed below, wherein one or more of the recombinant host cells (i) can be contacted with one or more ergoline precursor compounds, (ii) can be engineered to produce one or more ergoline precursor compounds, or a combination of (i) and (ii) in order to provide a method for generating an ergoline compound. Thus, the disclosure contemplates and provides methods comprising co-culturing of a plurality of cells, including a plurality of recombinant cells of the disclosure, in biosynthetic methods for generating an ergoline.

### Bioconversion Methods

Ergolines may be produced by a bioconversion method that comprises contacting or adding a precursor molecule to a cell culture under conditions that are effective for the cells to convert the precursor molecule to the final target such as, for example, an ergopeptine. The added precursor molecule in some embodiments can be an intermediate molecule in the biosynthetic pathway. In some embodiments, the precursor molecule may be from a commercial source, chemically synthesized, or produced and optionally purified from another organism, such as a fungus, plant, or microbe (bacterium or yeast).

As discussed above, precursor molecules, including precursor ergoline compounds, can be provided by one or a plurality of cells, including recombinant cells in accordance with the disclosure. The cells and/or recombinant cell(s) of the disclosure may comprise genes that are sufficient for generating an ergoline precursor and can be either co-cultured, or harvested (e.g., culture media, cells, lysed cells, etc.) to provide the ergoline precursor. The precursor(s) can be contacted with one or more additional cells, including the recombinant cells of the disclosure, under conditions that provide for the generation of an ergoline, or another precursor or metabolite thereof that can be used in continued/further bioconversion process steps.

In various embodiments, bioconversion of a precursor molecule may be carried out with live cells, with a mixture of live cells of different species, or with lysate from cell culture. Lysing the cells can allow enzymes to operate in a different local environment and allow free access of enzymes to substrate at all steps of the biosynthetic pathway with no barriers caused by subcellular localization. Illustrative combinations of cell and/or cell-free bioconversions effective in the biosynthesis of ergoline products are depicted in FIGS. 6 A, B, C, and D.

### Semi-Synthetic Methods

Another way to complete production of ergolines is to make some parts of the molecule using biosynthetic techniques and other parts using the techniques of synthetic organic chemistry. For example, the ring structure of the ergoline molecule could be made biosynthetically, then purified. Oxidations could then be carried out synthetically to generate the finished molecule. Alternately, some of the ring structures in the finished molecule could be made biosynthetically and other rings added using a chemically derived starting molecule and organic synthesis methods to attach the two pieces of the molecule together.

In some embodiments, peptide-type ergots, such as ergopeptines derived from fermentation of recombinant hosts expressing the ergoline biosynthesis pathway, are treated by acid esterification, yielding products such as dihydrolysergic acid methylester.

### Ergoline Analogs

Introduction of enzymes with functionalities outside the canonical ergoline pathway facilitates creation of analog molecules. These include acetyltransferases, methylases, hydroxylases, cytochrome P450s, nitrosylases, halogenases, prenyltransferases, alkyltransferases, acetyltransferases, cyclases, dehydratases, laccases, lactonases, reductases, oxidases, kinases, glycosylases, and/or glucuronidases. Expression of these enzymes can provide for the generation of ergoline molecules such as ergot glycosides and halogenated lysergic acid amide analogs, as well as other designed and targeted analogs.

In some embodiments, the enzymes which metabolize ergolines such as lysergic acid diethylamide (LSD) are expressed to generate ergoline analogs. These enzymes, such as those mentioned above, including glucuronosyltransferases, CYP P450s, oxidoreductases, lyases, peroxidases, amidases, and/or dehydrogenases are expressed to yield versions of 13-hydroxy-LSD, 14-hydroxy-LSD, lysergic acid ethyl-2-hydroxyethylamide, lysergic acid ethylvinylamide, 2-oxo-LSD, 2-oxo-3-hydroxy LSD, N-desmethyl-LSD (Nor-LSD), lysergic acid monoethylamide, 13-hydroxy-LSD-glucuronide, and/or 14-hydroxy-LSD-glucuronide.

### Halogenation and Glycosylation

Halogenase enzymes add a halogen ion such as fluorine, chlorine, iodine or bromine to a substrate molecule, and are known to be able to halogenate tryptophan-based substrates. Embodiments of the disclosure provide for expression of one or more active halogenases in the recombinant host cell, or as a co-culture as described herein, to produce halogenated ergoline analogs.

Similarly, glycosyltransferase enzymes can add a sugar, sugar acid, or carbohydrate group onto an ergoline or precursor or metabolite thereof produced in the recombinant host cell or as a co-culture, as described herein. Embodiments of the disclosure provide for expression of one or more active glycosyltransferases to produce glycosylated derivatives that can exhibit desirable properties associated with solubility, absorption, distribution, metabolism, and excretion (ADME) relative to a non-glycosylated form.

The following Examples illustrate several aspects and embodiments in accordance with the disclosure and in no way serve to limit the scope or spirit of the claims.

### EXAMPLES

### Example 1—Modified S. cerevisiae Strains Having Increased Tryptophan Accumulation

An engineered Saccharomyces cerevisiae strain is generated to accumulate increased intracellular amounts of tryptophan by deleting one or more pyruvate decarboxylase genes ARO10, PDC5, or PDC6. ARO10 encodes a phenylpyruvate decarboxylase that is involved in the catabolism of aromatic amino acids such as L-tryptophan. TheARO10 gene is deleted by replacing it with a URA3 cassette in the recombinant host. AARO10ΔURA3 knockout fragment carrying the marker cassette, URA3, and homologous sequence to the targeted gene, ARO10, is generated by bipartite PCR amplification. The resulting PCR product is transformed into a recombinant host and transformants are selected on synthetic URA drop-out media. Further verification of the modified strain can be carried out by genome sequencing, and/or further available techniques (see, e.g., U.S. Pat. No. 10,671,632).

### Example 2—Expression of Recombinant Chanoclavine Synthase and Catalase in a Modified Host Organism

A Saccharomyces cerevisiae strain is generated for the biosynthesis of chanoclavine via co-expression of the EASE chanoclavine synthase gene (as SEQ ID NO: 2) with the EASC catalase gene (as SEQ ID NOs: 12 or 13). The EASE and EASC genes are synthesized using DNA synthesis techniques known in the art, and are cloned into a vector. Suitable vectors can include an inducible vector containing yeast and bacterial selection markers and origins of replication, or in particular, a pESC series vector (Agilent, Santa Clara, CA, 95051 USA) that includes regulatory elements for gene expression (e.g. promoter, terminator), and a plasmid with a dual expression cassette for expressing both genes on a single episomal plasmid. The derived plasmid is confirmed by DNA sequencing.

Alternatively, the above EASE and EASC genes are inserted into the recombinant host genome, in a ratio of EasE:EasC ranging from 1:3 to 1:10. Integration is achieved by a single cross-over insertion event of the plasmid or linear donor containing the expression cassettes for the EASE and EASC genes. Optionally, additional copies of the EASC gene can be added by additional rounds of integration at a second genomic locus. Strains with the integrated genes are screened by rescue of auxotrophy and genome sequencing.

### Example 3—Method of Growth

The Saccharomyces cerevisiae strain described above is cultured under conditions that are effective to produce chanoclavine-producing strain herein is grown in a culture media. Briefly, the culture is grown and fermented in media containing 40 g/L glucose, 15 g/L ammonium sulfate, 2 g/L yeast nitrogen base (YNB), 1 g/L complete minimal dropout media (CSM) supplement, buffered with 60 mM KH₂PO₄ and 10 mM K₂HIPO₄, for 48 hrs in an incubator shaking at 300 RPM. After fermentation, the broth is extracted.

### Example 4—Detection of Fermentation Derived Chanoclavine

To identify fermentation-derived chanoclavine, an Agilent 1100 series liquid chromatography (LC) system equipped with a HILIC column (Primesep 100, SIELC, Wheeling, IL USA) is used. A gradient is used of mobile phase A (ultraviolet (UV) grade H₂O+0.2% TFA) and mobile phase B (UV grade acetonitrile+0.2% TFA), and the column temperature is set at 40° C. Compound absorbance is measured in a range of 218-275 nm using a diode array detector (DAD) and spectral analysis from 200 nm to 400 nm wavelengths. A 0.1 milligram (mg)/milliliter (mL) analytical standard is made from a certified reference material (Cayman Chemical Company, USA). Each sample is prepared by diluting fermentation biomass 1:1 in 100% ethanol and filtered in 0.2 um nanofilter vials. The retention time and UV-visible absorption spectrum (i.e., spectral fingerprints) of the samples are compared to the analytical standard retention time and UV-visible spectra (i.e. spectral fingerprint) to identify chanoclavine. FIG. 7 depicts the detection of chanoclavine isolated from a fermentation with a recombinant host. Detection and isolation are depicted by retention time matching of fermentation derived product with an analytical standard.

An EASE chanoclavine synthase gene (SEQ ID NO: 2) is combined with an EASC catalase gene (SEQ ID NO: 12) which are both synthesized using DNA synthesis techniques known in the art, and are cloned into a vector containing yeast and bacterial selection markers and origins of replication or, in particular, a pESC series vector that includes regulatory elements for gene expression (e.g. promoter, terminator), and a plasmid with a dual expression cassette for expressing both genes on a single episomal plasmid. The derived plasmid is confirmed by DNA sequencing.

### Example 5—Detection of Fermentation Derived Agroclavine

A Saccharomyces cerevisiae strain is generated for the biosynthesis of agroclavine via co-expression of the EASG (SEQ ID NO: 41, SEQ ID NO: 44, or SEQ ID NO: 45), EASD (SEQ ID NO: 20, SEQ ID NO: 25, or SEQ ID NO: 26), and EASA (SEQ ID NO: 31, SEQ ID NO: 34, SEQ ID NO: 35, or SEQ ID NO: 36) genes. Briefly, the recombinant S. cerevisiae cells are grown and fermented in media containing 40 g/L glucose, 15 g/L ammonium sulfate, 2 g/L yeast nitrogen base (YNB), 1 g/L complete minimal dropout media (CSM) supplement, buffered with 60 mM KH₂PO₄ and 10 mM K₂HPO₄ at 30° C. for 24-48 hrs to produce agroclavine.

To detect the presence of fermentative-derived agroclavine, LC-MS/MS in positive mode is used with a ThermoFisher LTQ XL mass spectrometer. Separation of agroclavine is achieved using an H₂O/ACN, 0.2% trifluoroacetic acid gradient on a HILIC amide column (FIG. 8). An agroclavine analytical standard is analyzed (at 0.1 mg/ml concentration) on a full scan, MS2 data-dependent method to determine the parent mass and characteristic MS2 fragmentation pattern of agroclavine. The parent mass is determined as 239.2, MW+1, and analysis of fermented strain samples is set for MS/MS at 239.2+/−1.5. The LC retention time and MS2 fragmentation pattern of the fermentation samples is compared to the agroclavine standard for a match factor, and subsequent positive identification. Agroclavine is not detected in negative control samples of a media blank or from fermentation media for a strain not expressing combinations of EASG, EASD and/or EASA enzymes.

### Example 6—Biosynthesis of Lysergic Acid and Lysergic Acid Amide

The agroclavine-producing strain described above is modified to express additional EAS and associated genes in accordance with the disclosure and encoding for enzymes with oxidase (CLOA), cofactor regeneration (CPR), peptidyl transferase (LPS), hydrolase (EASP), and oxidative peptidase activities (EASH, and EASO). Expression of the CLOA oxidase together with a CPR converts agroclavine into lysergic acid. Lysergic acid amide (LSA) is generated from lysergic acid via expression of the LPS to form a peptide linkage, which is cleaved by EASP, EASO, and EASH to yield LSA, such as depicted in FIG. 3A.

This modified strain is grown and fermented in media containing 40 g/L glucose, 15 g/L ammonium sulfate, 2 g/L yeast nitrogen base (YNB), 1 g/L complete minimal dropout media (CSM) supplement, buffered with 60 mM KH₂PO₄ and 10 mM K₂HIPO₄ at 30° C. for 24-48 hrs. Fermentation derived products are analyzed by diluting recovered fermentation broth 1:1 in 100% ethanol and filtering in 0.2 um nanofilter vials by one or both of HPLC and LC-MS analysis, using reference standards for confirmation of the fermentation derived end product.

### Example 7—Engineering of Strains with Upregulated Tryptophan Biosynthesis

Enzymes of the tryptophan biosynthesis pathway are upregulated by integration of additional copies of one or more of the TRP1, TRP2, TRP3, TRP4, TRP5 genes into the genome of the host strain. Genes are placed under the control of strong constitutive promoters. The strain is preferably prototrophic for tryptophan when genetic manipulations are complete.

### Example 8—Engineering of Strains with Increased Intracellular Levels of the Redox Cofactors NADPH and FAD

To increase levels of NADPH, ergoline producing strains are engineered to have additional copies of the ZWF1 gene integrated into the chromosome at one or more sites. ZWF1 is under the control of a constitutive promoter. Additionally, to increase levels of FAD, cells are engineered to have increased gene dosage of FAD1 and FMN1. Genes may be maintained on a high copy episomal plasmid or integrated into the genome at multiple sites.

### Example 9—Production of Ergoline Molecules by Co-Culture of Two or More Organisms

Two or more organisms, at least one of which is recombinantly engineered to express at least part of the ergoline biosynthesis pathway are inoculated into 5 mL growth media using sterile technique. Cells are cultured until OD >1 and then cultures are mixed together at ratios ranging from 1:1 to 1:10. Fresh nutrients are added, and conditions are altered for production of pathway enzymes. The resulting fermentation products are analyzed for the presence of the target ergoline molecules.

### Example 10—Fermentation of Engineered Strains

Cells are grown in media containing carbon and nitrogen feedstocks for 16-72 hrs at 30° C. with agitation from 100-400 rpm. Feedstocks can contain as a carbon source 10-80 g/L of either or combinations of glucose, sucrose, corn syrup, fats, oils, maltose, xylose, lactose, galactose and/or glycerol. Feedstocks can contain as a nitrogen source 5-50 g/L of either or combinations of ammonium sulfate, urea, cornsteep liquor, flour, wheat bran, and/or aquaculture waste. When the carbon source is exhausted, fresh nutrients, including the feedstocks are added along with pathway inducers and additives or cofactors required to support the production and activity of pathway enzymes. Other additives in the replenished media can include 5-50 g/L of either or combinations of L-tryptophan, L-serine, L-threonine, L-cysteine, L-methionine, L-proline, L-glutamine, and/or L-glycine.

Fermentation is conducted at temperatures between 10 and 40° C. with agitation between 100-400 rpm. Fermentation is maintained for 48-144 hrs. During the fermentation the pH is maintained between 4 and 6. Additives to control the redox state of the fermentation broth, including DTT, BME and/or TCEP are added in concentrations from 0.1-10 mM.

### Example 11—Bioconversion of Pathway Intermediates

Cells expressing a portion of the ergoline pathway are cultured until OD>1. Fresh media is added along with pathway inducers and additives, or cofactors required to support the production and activity of pathway enzymes. In addition, a pathway intermediate is added to the culture. Depending on solubility, a co solvent may be required to ensure the intermediate is soluble in the aqueous culture. Acceptable cosolvents include ethanol, isopropanol, DMSO, and acetone at concentrations ranging from 0.1% to 10%. Surfactants may also help to emulsify the mixture and keep the intermediate in solution. Acceptable surfactants include TritonX-100, Tween-20, Tween-80, NP-40, sodium deoxycholate

Cells may also be lysed after induction and the pathway intermediate added to the cell lysate for bioconversion. Lysis may occur by spheroplasting (addition of 0.1 mg/mL recombinant zymolyase in supplied buffer), or by processing through a bead mill, Dounce homogenizer or French press.

### Example 12—Semisynthetic Approach

Ergoline pathway intermediates are generated by fermentation in engineered cells, extracted and purified. Conversion to final products is accomplished by use of organic chemistry methods. For example, the Sharpless asymmetric dihydroxylation reaction can be used to convert agroclavine to a dihydroxylated version of elymoclavine.

1.-19. (canceled)

20. An expression vector comprising a polynucleotide sequence comprising SEQ ID NO: 1-SEQ ID NO: 138 or a sequence having about 95% sequence identity thereto, and an operatively-linked promoter sequence that allows for expression of the one or more polynucleotide sequences in a cell.

21. A recombinant cell comprising the expression vector of claim 20.

22. The recombinant cell according to claim 21, wherein the cell comprises an EASE polynucleotide sequence of SEQ ID NOs 1-11; and an EASC polynucleotide sequence of SEQ ID NOs 12-19.

23. The recombinant cell according to claim 22, wherein the cell is adapted to provide a stoichiometry of EASE polynucleotide sequence to EASC polynucleotide sequence of about 1:3 to about 1:10.

24. The recombinant cell according to claim 22, wherein the cell further comprises an EASD polynucleotide sequence of SEQ ID NOs 20-29.

25. The recombinant cell according to claim 21, wherein the cell comprises at least two of an EASG polynucleotide sequence of SEQ ID NOs 39-47, an EASA polynucleotide sequence of SEQ ID NOs 30-38, and an EASD polynucleotide sequence SEQ ID NOs 20-29.

26. The recombinant cell according to claim 25, wherein the cell comprises an EASG polynucleotide sequence, an EASA polynucleotide sequence, and an EASD polynucleotide sequence.

27. The recombinant cell according to claim 21, wherein the cell comprises an EASE polynucleotide sequence of SEQ ID NOs 1-11 and an EASC polynucleotide sequence of SEQ ID NOs 12-19, in combination with at least two of an EASG polynucleotide sequence of SEQ ID NOs 39-47, an EASA polynucleotide sequence of SEQ ID NOs 30-38, and an EASD polynucleotide sequence of SEQ ID NOs 20-29.

28. The recombinant cell according to claim 27, wherein the cell comprises an EASE, an EASC, an EASG, an EASA, and an EASD polynucleotide sequence.

29. The recombinant cell according to claim 21, further comprising one or more of an EASM polynucleotide sequence; a CLOA polynucleotide sequence; an LPS polynucleotide sequence; an EASO polynucleotide sequence; an EASP polynucleotide sequence; an EASH polynucleotide sequence; a CYP polynucleotide sequence; a CPR polynucleotide sequence; a CYB polynucleotide sequence; a HAL polynucleotide sequence, or a UGT polynucleotide sequence.

30. The recombinant cell according to claim 21, wherein the cell comprises a bacterial cell, a fungal cell, or a yeast cell.

31. The recombinant cell according to claim 30, wherein the cell is a species of yeast that includes Saccharomyces, Candida, Pichia, Schizosaccharomyces, Scheffersomyces, Blakeslea, Rhodotorula, or Yarrowia.

32. The recombinant cell according to claim 30, wherein the cell is a species of a filamentous fungus that includes Aspergillus or Penicillium.

33. The recombinant cell according to claim 30, wherein the cell is a species of bacteria that includes Escherichia, Corynebacterium, Caulobacter, Pseudomonas, Streptomyces, Bacillus, or Lactobacillus.

34. A method for the biosynthesis of an ergoline, or a precursor or metabolite thereof, the method comprising culturing a recombinant cell of claim 21 under conditions that allow for ergoline biosynthesis.

35. The method according to claim 34, wherein the method generates chanoclavine or chanoclavine aldehyde.

36. The method according to claim 34, wherein the method generates agroclavine.

37. The method according to claim 34, wherein the method generates festuclavine.

38. The method according to claim 34, wherein the method generates elymoclavine or dihydro elymoclavine.

39. The method according to claim 34, wherein the method generates paspalic aldehyde, dihydro paspalic aldehyde, or paspalic acid.

40. The method according to claim 34, wherein the method generates lysergic acid or dihydro lysergic acid.

41. The method according to claim 34, wherein the method generates lysergic acid hydroxyethylamide, dihydro lysergic acid hydroxyethylamide, lysergic acid amide, dihydro lysergic acid amide, lysergic acid diethylamide, or dihydro lysergic acid diethylamide.

42. The method according to claim 34, wherein the method generates fumigaclavine A, fumigaclavine B, or fumigaclavine C.

43.-60. (canceled)
